(19) United States
(12) Patent Application Publication
(10) Pub. No.: US 2025/0262297 A1
(43) Pub. Date: Aug. 21, 2025

(54) ALLERGY ANTIGEN AND EPITOPE THEREOF

(71) Applicant: HOYU CO., LTD., Aichi (JP)

(72) Inventors: Kayoko Matsunaga, Aichi (JP); Akiko Yagami, Aichi (JP); Naoshi Shimojo, Aichi (JP); Fumiaki Ohno, Aichi (JP)

(21) Appl. No.: 19/036,628

(22) Filed: Jan. 24, 2025

Related U.S. Application Data
(62) Division of application No. 16/490,092, filed on Aug. 30, 2019.

(30) Foreign Application Priority Data
Mar. 3, 2017 (WO) PCT/JP2017/008593

(51) Int. Cl. A61K 39/35; A23L 11/30; A61K 38/00; A61K 49/00; A61P 37/08; C07K 4/10; C07K 14/415; C07K 14/47; G01N 33/53

(52) U.S. Cl. CPC A61K 39/35; A23L 11/30; A61K 49/00; A61K 49/0006; A61P 37/08; C07K 4/10; C07K 14/415; C07K 14/47; G01N 33/53; A61K 38/00; G01N 2800/24

(57) ABSTRACT

The present invention provides novel antigens of an allergy to soybean, methods and kits for diagnosing an allergy to soybean, pharmaceutical compositions comprising such an antigen, soybeans or processed products of soybean in which such an antigen is eliminated, and a tester for determining the presence or absence of a soybean antigen in an object of interest. The present invention also relates to polypeptides comprising an epitope of an allergen, kits, compositions and methods for diagnosing an allergy, comprising such a polypeptide, pharmaceutical compositions comprising such a polypeptide, and food raw materials or edible processed products in which an antigen comprising such a polypeptide is eliminated or reduced, or the polypeptide is cleaved or removed. The present invention further relates to a tester for determining the presence or absence of an antigen in an object of interest.

### RELATED APPLICATIONS

This application is a divisional application of U.S. patent application Ser. No. 16/490,092, filed on Aug. 30, 2019, which is a U.S. National Stage Application of International Patent Application No. PCT/JP2017/032005, filed on Sep. 5, 2017, which claims priority to International Patent Application No. PCT/JP2017/008593, filed on Mar. 3, 2017. The contents of each of the aforementioned applications, including sequence listings, are incorporated herein by reference in their entirety.

### REFERENCE TO SEQUENCE LISTING

The instant application contains a Sequence Listing which has been submitted electronically in XML format and is hereby incorporated by reference in its entirety. Said XML copy, created on May 5, 2025, is named “129249_00603_SL.xml” and is 1,159,840 bytes in size.

### TECHNICAL FIELD

The present invention relates to a novel antigen of an allergy to soybean. The present invention also relates to a kit, a composition, and a method for diagnosing allergy to soybean. The present invention also relates to a pharmaceutical composition comprising such an antigen and soybean or processed products of soybean in which such an antigen is eliminated or reduced. The present invention further relates to a tester for determining the presence or absence of a soybean antigen in an object of interest.

The present invention also relates to a polypeptide comprising an epitope of an allergen. The present invention also relates to a kit, a composition and a method for diagnosing an allergy, comprising such a polypeptide. The present invention also relates to a pharmaceutical composition comprising such a polypeptide, and a food raw material or an edible processed product in which an antigen comprising such a polypeptide is eliminated or reduced, or such a polypeptide is cleaved or removed. The present invention further relates to a method for producing an edible processed product in which such an antigen is eliminated or reduced. The present invention further relates to a tester for determining the presence or absence of an antigen comprising such a polypeptide in an object of interest.

### BACKGROUND ART

In blood and tissues of allergic patients, IgE antibodies specific to particular antigens are produced. Physiological consequences caused by interaction between such IgE antibodies and such particular antigens elicit allergic reactions.

In the process of production of conventional allergy testing agents, antigen reagents are commonly prepared simply by grinding a candidate allergenic food, material or the like (Patent Literature 1). As seen above, conventional antigen reagents do not necessarily contain only a particular antigenic protein inducing an allergic reaction (allergen component), and rather contain different types of protein components. Thus, conventional antigen reagents contain varied amounts of allergen components. For this reason, the only case where conventional allergy tests have permitted detection of a positive allergic reaction is when in a conventional antigen reagent containing many types of proteins, a particular protein acting as an allergen component is present in an amount exceeding a threshold that allows determination of a positive reaction for binding to an IgE antibody. However, no determination of a positive reaction was possible and diagnosis efficiency was not sufficiently high when using a conventional allergy testing agent in patients possessing an IgE antibody binding to an allergen component present in small amounts in an allergen such as food.

The severity and symptoms of an allergic reaction do not necessarily correlate with the content of an allergen component. Even when a patient's IgE antibody reacts with an allergen component present in trace amounts in a candidate allergic food and material, the allergic reaction may develop allergic symptoms or may affect the severity of those symptoms.

An attempt to increase the diagnostic efficiency is being made by examining IgE antibodies to components composing a crude antigen to distinguish sensitization that directly contributes to a diagnosis from sensitization based on cross-antigenicity by a pan-allergen or the like. 8 soybean allergens are currently registered to the World Health Organization/International Union of Immunological Societies (WHO/IUIS).

However, while it is necessary to exhaustively identify allergen components in candidate allergic foods and materials in order to enhance the reliability of allergy tests, the patient detection rate by the measurement of such allergenic components is far from sufficient. Identification of novel allergens in soybean is very important not only for increasing the precision of diagnosis, but also for determining targets of therapeutic agents and low allergenic foods.

Meanwhile, in the field of protein separation and purification, various efforts have conventionally been made to develop methods for separating and purifying a protein or nucleic acid of interest from cell extracts or the like. Such methods may well be exemplified by dialysis based on salt concentration, and centrifugal separation.

Other efforts have been made to develop many purification methods based on electric charges of protein or nucleic acid residues or on the difference in molecular weight. Electric charge-based purification methods can be exemplified by column chromatography using ion exchange resins, and isoelectric focusing. Purifications based on molecular weight difference can be exemplified by centrifugal separation, molecular-sieve column chromatography, and SDS-PAGE (sodium dodecyl sulfate-polyacrylamide gel electrophoresis).

In recent years, a method for separating and purifying many different proteins from a small amount of sample has been used, which is more specifically a two-dimensional electrophoresis consisting of isoelectric focusing in the first dimension, followed by SDS-PAGE in the second dimension. The present applicant has conventionally developed some 2D electrophoresis methods with high separation ability (Patent Literature 2-5).

Allergen-specific IgE antibodies recognize and bind to epitopes that are particular amino acid sequences in allergen components. However, only a slight number of analyses have been made on epitopes as to the allergen components (Non Patent Literature 1), but such analyses are still totally quite rare. Furthermore, any kit for diagnosing an allergy using a polypeptide comprising an epitope has not yet emerged in the market.

### CITATION LIST

### Patent Literature

PTL1: Japanese Patent Application Publication No. JP 2002-286716
PTL2: Japanese Patent Application Publication No. JP 2011-33544
PTL3: Japanese Patent Application Publication No. JP 2011-33546
PTL4: Japanese Patent Application Publication No. JP 2011-33547
PTL5: Japanese Patent Application Publication No. JP 2011-33548

### Non Patent Literature

NPL 1: Matsuo, H., et al., J. Biol. Chem., (2004), Vol.279, No. 13, pp. 12135-12140
NPL 2: Zhao, L., et al., PLOS One. 2017 Aug. 11; 12 (8): c0182935. doi: 10.1371/journal.pone.0182935. eCollection 2017.
NPL 3: Cong, Y., et al., J Dairy Sci. 2013; 96 (11): 6870-6. doi: 10.3168/jds.2013-6880. Epub 2013 Sep. 12.
NPL 4: Beczhold, D. H., et al., J Allergy Clin Immunol. 2001 June; 107 (6): 1069-76.

### SUMMARY OF INVENTION

### Technical Problem

The present invention provides novel antigens of an allergy to soybean. The present invention also provides methods and kits for diagnosing allergy to soybean. The present invention also provides pharmaceutical compositions comprising such an antigen and soybean or processed products of soybean in which such an antigen is eliminated or reduced. The present invention further provides testers for determining the presence or absence of a soybean antigen in an object of interest.

The present invention also provides polypeptides comprising an epitope of an allergen. The present invention also provides kits, compositions and methods for diagnosing an allergy. The present invention also provides pharmaceutical compositions comprising such a polypeptide, and food raw materials or edible processed products in which an antigen comprising such a polypeptide is eliminated or reduced, or such a polypeptide is cleaved or removed. The present invention further relates to methods for producing an edible processed product in which such an antigen is eliminated or reduced. The present invention further relates to testers for determining the presence or absence of an antigen comprising such a polypeptide in an object of interest.

### Solution to Problem

In order to solve the aforementioned problems, the present inventors had made intensive studies to identify causative antigens of an allergy. As a result, the inventors succeeded in identifying novel antigens to which an IgE antibody in the serum of an allergic patient specifically binds. The present invention has been completed based on this finding.

Thus, in one embodiment, the present invention can be as defined below.

[1] A kit for diagnosing an allergy to a soybean, the kit comprising, as an antigen, at least one of proteins defined below in any one of (1a) to (55a):

(1α) (1αA1) endoplasmin homolog isoform X2 or a variant thereof, which is defined below in any of (1αA1-a) to (1αA1-e):
(1αA1-a) a protein comprising an amino acid sequence with deletion, substitution, insertion or addition of one or several amino acids in SEQ ID NO: 70;
(1αA1-b) a protein comprising an amino acid sequence having at least 70% identity to the amino acid sequence of SEQ ID NO: 70;
(1αA1-c) a protein comprising an amino acid sequence encoded by a nucleotide sequence with deletion, substitution, insertion or addition of one or several nucleotides in SEQ ID NO: 69;
(1αA1-d) a protein comprising an amino acid sequence encoded by a nucleotide sequence having at least 70% identity to the nucleotide sequence of SEQ ID NO: 69; or
(1αA1-e) a protein comprising an amino acid sequence encoded by a nucleic acid that hybridizes under stringent conditions with a nucleic acid having a nucleotide sequence complementary to the nucleotide sequence of SEQ ID NO: 69;
(1aB1) a protein comprising at least one amino acid sequence selected from the group consisting of SEQ ID NOs: 71-87,
(1αA2) endoplasmin homolog isoform X2 or a variant thereof, which is defined below in any of (1αA2-a) to (1αA2-e):
(1αA2-a) a protein comprising an amino acid sequence with deletion, substitution, insertion or addition of one or several amino acids in SEQ ID NO: 89;
(1αA2-b) a protein comprising an amino acid sequence having at least 70% identity to the amino acid sequence of SEQ ID NO: 89;
(1αA2-c) a protein comprising an amino acid sequence encoded by a nucleotide sequence with deletion, substitution, insertion or addition of one or several nucleotides in SEQ ID NO: 88;
(1αA2-d) a protein comprising an amino acid sequence encoded by a nucleotide sequence having at least 70% identity to the nucleotide sequence of SEQ ID NO: 88; or
(1αA2-e) a protein comprising an amino acid sequence encoded by a nucleic acid that hybridizes under stringent conditions with a nucleic acid having a nucleotide sequence complementary to the nucleotide sequence of SEQ ID NO: 88; or
(1αB2) a protein comprising at least one amino acid sequence selected from the group consisting of SEQ ID NOs: 90-104;
   (2α) (2αA1) heat shock cognate protein 80 or a variant thereof, which is defined below in any of (2αA1-a) to (2αA1-e):
(2αA1-a) a protein comprising an amino acid sequence with deletion, substitution, insertion or addition of one or several amino acids in SEQ ID NO: 106;
(2αA1-b) a protein comprising an amino acid sequence having at least 70% identity to the amino acid sequence of SEQ ID NO: 106;
(2αA1-c) a protein comprising an amino acid sequence encoded by a nucleotide sequence with deletion, substitution, insertion or addition of one or several nucleotides in SEQ ID NO: 105;
(2αA1-d) a protein comprising an amino acid sequence encoded by a nucleotide sequence having at least 70% identity to the nucleotide sequence of SEQ ID NO: 105; or
(2αA1-e) a protein comprising an amino acid sequence encoded by a nucleic acid that hybridizes under stringent conditions with a nucleic acid having a nucleotide sequence complementary to the nucleotide sequence of SEQ ID NO: 105; or
(2αB1) a protein comprising at least one amino acid sequence selected from the group consisting of SEQ ID NOs: 107-120;
(2αA2) heat shock protein 90-1 or a variant thereof, which is defined below in any of (2αA2-a) to (2αA2-e):
(2αA2-a) a protein comprising an amino acid sequence with deletion, substitution, insertion or addition of one or several amino acids in SEQ ID NO: 122;
(2A2-b) a protein comprising an amino acid sequence having at least 70% identity to the amino acid sequence of SEQ ID NO: 122;
(2αA2-c) a protein comprising an amino acid sequence encoded by a nucleotide sequence with deletion, substitution, insertion or addition of one or several nucleotides in SEQ ID NO: 121;
(2αA2-d) a protein comprising an amino acid sequence encoded by a nucleotide sequence having at least 70% identity to the nucleotide sequence of SEQ ID NO: 121; or
(2αA2-e) a protein comprising an amino acid sequence encoded by a nucleic acid that hybridizes under stringent conditions with a nucleic acid having a nucleotide sequence complementary to the nucleotide sequence of SEQ ID NO: 121; or
(2αB2) a protein comprising at least one amino acid sequence selected from the group consisting of SEQ ID NOs: 123-137;
(2αA3) hypothetical protein GLYMA_02G302500 or a variant thereof, which is defined below in any of (2αA3-a) to (2αA3-e):
(2αA3-a) a protein comprising an amino acid sequence with deletion, substitution, insertion or addition of one or several amino acids in SEQ ID NO: 139;
(2αA3-b) a protein comprising an amino acid sequence having at least 70% identity to the amino acid sequence of SEQ ID NO: 139;
(2αA3-c) a protein comprising an amino acid sequence encoded by a nucleotide sequence with deletion, substitution, insertion or addition of one or several nucleotides in SEQ ID NO: 138;
(2αA3-d) a protein comprising an amino acid sequence encoded by a nucleotide sequence having at least 70% identity to the nucleotide sequence of SEQ ID NO: 138; or
(2αA3-e) a protein comprising an amino acid sequence encoded by a nucleic acid that hybridizes under stringent conditions with a nucleic acid having a nucleotide sequence complementary to the nucleotide sequence of SEQ ID NO: 138; or
(2αB3) a protein comprising at least one amino acid sequence selected from the group consisting of SEQ ID NOs: 140-153;
   (4α) (4aA) embryo-specific urease isoform X1 or a variant thereof, which is defined below in any of (4αA-a) to (4αA-e):
(4αA-a) a protein comprising an amino acid sequence with deletion, substitution, insertion or addition of one or several amino acids in SEQ ID NO: 155;
(4αA-b) a protein comprising an amino acid sequence having at least 70% identity to the amino acid sequence of SEQ ID NO: 155;
(4αA-c) a protein comprising an amino acid sequence encoded by a nucleotide sequence with deletion, substitution, insertion or addition of one or several nucleotides in SEQ ID NO: 154;
(4αA-d) a protein comprising an amino acid sequence encoded by a nucleotide sequence having at least 70% identity to the nucleotide sequence of SEQ ID NO: 154; or
(4αA-e) a protein comprising an amino acid sequence encoded by a nucleic acid that hybridizes under stringent conditions with a nucleic acid having a nucleotide sequence complementary to the nucleotide sequence of SEQ ID NO: 154; or
(4αB) a protein comprising at least one amino acid sequence selected from the group consisting of SEQ ID NOs: 156 and 157;
   (5α) (5αA) alpha-1,4 glucan phosphorylase L isozyme, chloroplastic/amyloplastic or a variant thereof, which is defined below in any of (5αA-a) to (5αA-e):
(5αA-a) a protein comprising an amino acid sequence with deletion, substitution, insertion or addition of one or several amino acids in SEQ ID NO: 159;
(5αA-b) a protein comprising an amino acid sequence having at least 70% identity to the amino acid sequence of SEQ ID NO: 159;
(5αA-c) a protein comprising an amino acid sequence encoded by a nucleotide sequence with deletion, substitution, insertion or addition of one or several nucleotides in SEQ ID NO: 158;
(5αA-d) a protein comprising an amino acid sequence encoded by a nucleotide sequence having at least 70% identity to the nucleotide sequence of SEQ ID NO: 158; or
(5αA-e) a protein comprising an amino acid sequence encoded by a nucleic acid that hybridizes under stringent conditions with a nucleic acid having a nucleotide sequence complementary to the nucleotide sequence of SEQ ID NO: 158; or
(5αB) a protein comprising at least one amino acid sequence selected from the group consisting of SEQ ID NOs: 160 and 161;
   (6α) (6αA1) aconitate hydratase 1 or a variant thereof, which is defined below in any of (6αA1-a) to (6αA1-e):
(6αA1-a) a protein comprising an amino acid sequence with deletion, substitution, insertion or addition of one or several amino acids in SEQ ID NO: 163;
(6αA1-b) a protein comprising an amino acid sequence having at least 70% identity to the amino acid sequence of SEQ ID NO: 163;
(6αA1-c) a protein comprising an amino acid sequence encoded by a nucleotide sequence with deletion, substitution, insertion or addition of one or several nucleotides in SEQ ID NO: 162;
(6αA1-d) a protein comprising an amino acid sequence encoded by a nucleotide sequence having at least 70% identity to the nucleotide sequence of SEQ ID NO: 162; or
(6αA1-e) a protein comprising an amino acid sequence encoded by a nucleic acid that hybridizes under stringent conditions with a nucleic acid having a nucleotide sequence complementary to the nucleotide sequence of SEQ ID NO: 162;
(6αB1) a protein comprising at least one amino acid sequence selected from the group consisting of SEQ ID NOs: 164-167; or
(6αA2) aconitate hydratase 1 or a variant thereof, which is defined below in any of (6αA2-a) to (6αA2-e):
(6αA2-a) a protein comprising an amino acid sequence with deletion, substitution, insertion or addition of one or several amino acids in SEQ ID NO: 169;
(6αA2-b) a protein comprising an amino acid sequence having at least 70% identity to the amino acid sequence of SEQ ID NO: 169;
(6αA2-c) a protein comprising an amino acid sequence encoded by a nucleotide sequence with deletion, substitution, insertion or addition of one or several nucleotides in SEQ ID NO: 168;
(6αA2-d) a protein comprising an amino acid sequence encoded by a nucleotide sequence having at least 70% identity to the nucleotide sequence of SEQ ID NO: 168; or
(6αA2-e) a protein comprising an amino acid sequence encoded by a nucleic acid that hybridizes under stringent conditions with a nucleic acid having a nucleotide sequence complementary to the nucleotide sequence of SEQ ID NO: 168; or
(6B2) a protein comprising at least one amino acid sequence selected from the group consisting of SEQ ID NOs: 170-172;
   (7α) (7αA) methionine synthase or a variant thereof, which is defined below in any of (7αA-a) to (7αA-e):
(7αA-a) a protein comprising an amino acid sequence with deletion, substitution, insertion or addition of one or several amino acids in SEQ ID NO: 174;
(7αA-b) a protein comprising an amino acid sequence having at least 70% identity to the amino acid sequence of SEQ ID NO: 174;
(7αA-c) a protein comprising an amino acid sequence encoded by a nucleotide sequence with deletion, substitution, insertion or addition of one or several nucleotides in SEQ ID NO: 173;
(7αA-d) a protein comprising an amino acid sequence encoded by a nucleotide sequence having at least 70% identity to the nucleotide sequence of SEQ ID NO: 173; or
(7αA-e) a protein comprising an amino acid sequence encoded by a nucleic acid that hybridizes under stringent conditions with a nucleic acid having a nucleotide sequence complementary to the nucleotide sequence of SEQ ID NO: 173; or
(7αB) a protein comprising at least one amino acid sequence selected from the group consisting of SEQ ID NOs: 175-187;
   (8%) (8αA) transketolase, chloroplastic or a variant thereof, which is defined below in any of (8αA-a) to (8αA-e):
(8αA-a) a protein comprising an amino acid sequence with deletion, substitution, insertion or addition of one or several amino acids in SEQ ID NO: 189;
(8αA-b) a protein comprising an amino acid sequence having at least 70% identity to the amino acid sequence of SEQ ID NO: 189;
(8αA-c) a protein comprising an amino acid sequence encoded by a nucleotide sequence with deletion, substitution, insertion or addition of one or several nucleotides in SEQ ID NO: 188;
(8αA-d) a protein comprising an amino acid sequence encoded by a nucleotide sequence having at least 70% identity to the nucleotide sequence of SEQ ID NO: 188; or
(8αA-e) a protein comprising an amino acid sequence encoded by a nucleic acid that hybridizes under stringent conditions with a nucleic acid having a nucleotide sequence complementary to the nucleotide sequence of SEQ ID NO: 188; or
(8αB) a protein comprising at least one amino acid sequence selected from the group consisting of SEQ ID NOs: 190-199;
   (9α) (9αA) lysine-tRNA ligase, cytoplasmic-like isoform X2 or a variant thereof, which is defined below in any of (9αA-a) to (9αA-e):
(9αA-a) a protein comprising an amino acid sequence with deletion, substitution, insertion or addition of one or several amino acids in SEQ ID NO: 201;
(9αA-b) a protein comprising an amino acid sequence having at least 70% identity to the amino acid sequence of SEQ ID NO: 201;
(9αA-c) a protein comprising an amino acid sequence encoded by a nucleotide sequence with deletion, substitution, insertion or addition of one or several nucleotides in SEQ ID NO: 200;
(9αA-d) a protein comprising an amino acid sequence encoded by a nucleotide sequence having at least 70% identity to the nucleotide sequence of SEQ ID NO: 200; or
(9αA-e) a protein comprising an amino acid sequence encoded by a nucleic acid that hybridizes under stringent conditions with a nucleic acid having a nucleotide sequence complementary to the nucleotide sequence of SEQ ID NO: 200; or
(9B) a protein comprising an amino acid sequence of SEQ ID NO: 202;
   (10α) (10αA1) chaperonin CPN60-2, mitochondrial or a variant thereof, which is defined below in any of (10αA1-a) to (10αA1-e):
(10αA1-a) a protein comprising an amino acid sequence with deletion, substitution, insertion or addition of one or several amino acids in SEQ ID NO: 204;
(10αA1-b) a protein comprising an amino acid sequence having at least 70% identity to the amino acid sequence of SEQ ID NO: 204;
(10αA1-c) a protein comprising an amino acid sequence encoded by a nucleotide sequence with deletion, substitution, insertion or addition of one or several nucleotides in SEQ ID NO: 203;
(10αA1-d) a protein comprising an amino acid sequence encoded by a nucleotide sequence having at least 70% identity to the nucleotide sequence of SEQ ID NO: 203; or
(10αA1-e) a protein comprising an amino acid sequence encoded by a nucleic acid that hybridizes under stringent conditions with a nucleic acid having a nucleotide sequence complementary to the nucleotide sequence of SEQ ID NO: 203;
(10αB1) a protein comprising at least one amino acid sequence selected from the group consisting of SEQ ID NOs: 205-218;
(10αA2) chaperonin CPN60-2, mitochondrial-like isoform X1 or a variant thereof, which is defined below in any of (10αA2-a) to (10αA2-e):
(10αA2-a) a protein comprising an amino acid sequence with deletion, substitution, insertion or addition of one or several amino acids in SEQ ID NO: 220;
(10αA2-b) a protein comprising an amino acid sequence having at least 70% identity to the amino acid sequence of SEQ ID NO: 220;
(10αA2-c) a protein comprising an amino acid sequence encoded by a nucleotide sequence with deletion, substitution, insertion or addition of one or several nucleotides in SEQ ID NO: 219;
(10αA2-d) a protein comprising an amino acid sequence encoded by a nucleotide sequence having at least 70% identity to the nucleotide sequence of SEQ ID NO: 219; or
(10αA2-e) a protein comprising an amino acid sequence encoded by a nucleic acid that hybridizes under stringent conditions with a nucleic acid having a nucleotide sequence complementary to the nucleotide sequence of SEQ ID NO: 219; or
(10αB2) a protein comprising at least one amino acid sequence selected from the group consisting of SEQ ID NOs: 221-231;
   (11α) (11αA) polyadenylate-binding protein 8 isoform X3 or a variant thereof, which is defined below in any of (11αA-a) to (11αA-e):
(11αA-a) a protein comprising an amino acid sequence with deletion, substitution, insertion or addition of one or several amino acids in SEQ ID NO: 233;
(11αA-b) a protein comprising an amino acid sequence having at least 70% identity to the amino acid sequence of SEQ ID NO: 233;
(11αA-c) a protein comprising an amino acid sequence encoded by a nucleotide sequence with deletion, substitution, insertion or addition of one or several nucleotides in SEQ ID NO: 232;
(11αA-d) a protein comprising an amino acid sequence encoded by a nucleotide sequence having at least 70% identity to the nucleotide sequence of SEQ ID NO: 232; or
(11αA-e) a protein comprising an amino acid sequence encoded by a nucleic acid that hybridizes under stringent conditions with a nucleic acid having a nucleotide sequence complementary to the nucleotide sequence of SEQ ID NO: 232; or
(11αB) a protein comprising at least one amino acid sequence selected from the group consisting of SEQ ID NOs: 234-237;
   (12α) (12αA) pyrophosphate-fructose 6-phosphate 1-phosphotransferase subunit alpha-like or a variant thereof, which is defined below in any of (12αA-a) to (12αA-e):
(12αA-a) a protein comprising an amino acid sequence with deletion, substitution, insertion or addition of one or several amino acids in SEQ ID NO: 239;
(12αA-b) a protein comprising an amino acid sequence having at least 70% identity to the amino acid sequence of SEQ ID NO: 239;
(12αA-c) a protein comprising an amino acid sequence encoded by a nucleotide sequence with deletion, substitution, insertion or addition of one or several nucleotides in SEQ ID NO: 238;
(12αA-d) a protein comprising an amino acid sequence encoded by a nucleotide sequence having at least 70% identity to the nucleotide sequence of SEQ ID NO: 238; or
(12αA-e) a protein comprising an amino acid sequence encoded by a nucleic acid that hybridizes under stringent conditions with a nucleic acid having a nucleotide sequence complementary to the nucleotide sequence of SEQ ID NO: 238; or
(12αB) a protein comprising an amino acid sequence of SEQ ID NO: 240;
   (13α) (13αA1) protein disulfide isomerase-like protein precursor or a variant thereof, which is defined below in any of (13αA1-a) to (13αA1-e):
(13αA1-a) a protein comprising an amino acid sequence with deletion, substitution, insertion or addition of one or several amino acids in SEQ ID NO: 242;
(13αA1-b) a protein comprising an amino acid sequence having at least 70% identity to the amino acid sequence of SEQ ID NO: 242;
(13αA1-c) a protein comprising an amino acid sequence encoded by a nucleotide sequence with deletion, substitution, insertion or addition of one or several nucleotides in SEQ ID NO: 241;
(13αA1-d) a protein comprising an amino acid sequence encoded by a nucleotide sequence having at least 70% identity to the nucleotide sequence of SEQ ID NO: 241; or
(13αA1-e) a protein comprising an amino acid sequence encoded by a nucleic acid that hybridizes under stringent conditions with a nucleic acid having a nucleotide sequence complementary to the nucleotide sequence of SEQ ID NO: 241;
(13αB1) a protein comprising at least one amino acid sequence selected from the group consisting of SEQ ID NOs: 243-252;
(13αA2) protein disulfide-isomerase or a variant thereof, which is defined below in any of (13αA2-a) to (13αA2-e):
(13αA2-a) a protein comprising an amino acid sequence with deletion, substitution, insertion or addition of one or several amino acids in SEQ ID NO: 254;
(13αA2-b) a protein comprising an amino acid sequence having at least 70% identity to the amino acid sequence of SEQ ID NO: 254;
(13αA2-c) a protein comprising an amino acid sequence encoded by a nucleotide sequence with deletion, substitution, insertion or addition of one or several nucleotides in SEQ ID NO: 253;
(13αA2-d) a protein comprising an amino acid sequence encoded by a nucleotide sequence having at least 70% identity to the nucleotide sequence of SEQ ID NO: 253; or
(13αA2-e) a protein comprising an amino acid sequence encoded by a nucleic acid that hybridizes under stringent conditions with a nucleic acid having a nucleotide sequence complementary to the nucleotide sequence of SEQ ID NO: 253; or
(13αB2) a protein comprising at least one amino acid sequence selected from the group consisting of SEQ ID NOs: 255-278;
   (14α) (14αA1) V-type proton ATPase subunit B 2 or a variant thereof, which is defined below in any of (14αA1-a) to (14αA1-e):
(14αA1-a) a protein comprising an amino acid sequence with deletion, substitution, insertion or addition of one or several amino acids in SEQ ID NO: 280;
(14αA1-b) a protein comprising an amino acid sequence having at least 70% identity to the amino acid sequence of SEQ ID NO: 280;
(14αA1-c) a protein comprising an amino acid sequence encoded by a nucleotide sequence with deletion, substitution, insertion or addition of one or several nucleotides in SEQ ID NO: 279;
(14αA1-d) a protein comprising an amino acid sequence encoded by a nucleotide sequence having at least 70% identity to the nucleotide sequence of SEQ ID NO: 279; or
(14αA1-e) a protein comprising an amino acid sequence encoded by a nucleic acid that hybridizes under stringent conditions with a nucleic acid having a nucleotide sequence complementary to the nucleotide sequence of SEQ ID NO: 279;
(14αB1) a protein comprising at least one amino acid sequence selected from the group consisting of SEQ ID NOs: 281-284;
(14αA2) V-type proton ATPase subunit B 2 or a variant thereof, which is defined below in any of (14αA2-a) to (14αA2-e):
(14αA2-a) a protein comprising an amino acid sequence with deletion, substitution, insertion or addition of one or several amino acids in SEQ ID NO: 286;
(14αA2-b) a protein comprising an amino acid sequence having at least 70% identity to the amino acid sequence of SEQ ID NO: 286;
(14αA2-c) a protein comprising an amino acid sequence encoded by a nucleotide sequence with deletion, substitution, insertion or addition of one or several nucleotides in SEQ ID NO: 285;
(14αA2-d) a protein comprising an amino acid sequence encoded by a nucleotide sequence having at least 70% identity to the nucleotide sequence of SEQ ID NO: 285; or
(14αA2-e) a protein comprising an amino acid sequence encoded by a nucleic acid that hybridizes under stringent conditions with a nucleic acid having a nucleotide sequence complementary to the nucleotide sequence of SEQ ID NO: 285; or
(14αB2) a protein comprising at least one amino acid sequence selected from the group consisting of SEQ ID NOs: 287-289;
   (15, 16) (15, 16αA1) UTP-glucose-1-phosphate uridylyltransferase or a variant thereof, which is defined below in any of (15, 16αA1-a) to (15, 16αA1-e):
(15, 16αA1-a) a protein comprising an amino acid sequence with deletion, substitution, insertion or addition of one or several amino acids in SEQ ID NO: 291;
(15, 16αA1-b) a protein comprising an amino acid sequence having at least 70% identity to the amino acid sequence of SEQ ID NO: 291;
(15, 16αA1-c) a protein comprising an amino acid sequence encoded by a nucleotide sequence with deletion, substitution, insertion or addition of one or several nucleotides in SEQ ID NO: 290;
(15, 16αA1-d) a protein comprising an amino acid sequence encoded by a nucleotide sequence having at least 70% identity to the nucleotide sequence of SEQ ID NO: 290; or
(15, 16αA1-e) a protein comprising an amino acid sequence encoded by a nucleic acid that hybridizes under stringent conditions with a nucleic acid having a nucleotide sequence complementary to the nucleotide sequence of SEQ ID NO: 290;
(15, 16αB1) a protein comprising at least one amino acid sequence selected from the group consisting of SEQ ID NOs: 292-304;
(15, 16αA2) hypothetical protein GLYMA_02G241100 or a variant thereof, which is defined below in any of (15, 16αA2-a) to (15, 16αA2-e):
(15, 16αA2-a) a protein comprising an amino acid sequence with deletion, substitution, insertion or addition of one or several amino acids in SEQ ID NO: 306;
(15, 16αA2-b) a protein comprising an amino acid sequence having at least 70% identity to the amino acid sequence of SEQ ID NO: 306;
(15, 16αA2-c) a protein comprising an amino acid sequence encoded by a nucleotide sequence with deletion, substitution, insertion or addition of one or several nucleotides in SEQ ID NO: 305;
(15, 16αA2-d) a protein comprising an amino acid sequence encoded by a nucleotide sequence having at least 70% identity to the nucleotide sequence of SEQ ID NO: 305; or
(15, 16αA2-e) a protein comprising an amino acid sequence encoded by a nucleic acid that hybridizes under stringent conditions with a nucleic acid having a nucleotide sequence complementary to the nucleotide sequence of SEQ ID NO: 305; or
(15, 16αB2) a protein comprising at least one amino acid sequence selected from the group consisting of SEQ ID NOs: 307-318;
   (17α) (17αA1) guanosine nucleotide diphosphate dissociation inhibitor 2 or a variant thereof, which is defined below in any of (17αA1-a) to (17αA1-e):
(17αA1-a) a protein comprising an amino acid sequence with deletion, substitution, insertion or addition of one or several amino acids in SEQ ID NO: 320;
(17αA1-b) a protein comprising an amino acid sequence having at least 70% identity to the amino acid sequence of SEQ ID NO: 320;
(17αA1-c) a protein comprising an amino acid sequence encoded by a nucleotide sequence with deletion, substitution, insertion or addition of one or several nucleotides in SEQ ID NO: 319;
(17αA1-d) a protein comprising an amino acid sequence encoded by a nucleotide sequence having at least 70% identity to the nucleotide sequence of SEQ ID NO: 319; or
(17αA1-e) a protein comprising an amino acid sequence encoded by a nucleic acid that hybridizes under stringent conditions with a nucleic acid having a nucleotide sequence complementary to the nucleotide sequence of SEQ ID NO: 319;
(17αB1) a protein comprising at least one amino acid sequence selected from the group consisting of SEQ ID NOs: 321-325;
(17αA2) rab GDP dissociation inhibitor alpha-like or a variant thereof, which is defined below in any of (17αA2-a) to (17αA2-e):
(17αA2-a) a protein comprising an amino acid sequence with deletion, substitution, insertion or addition of one or several amino acids in SEQ ID NO: 327;
(17αA2-b) a protein comprising an amino acid sequence having at least 70% identity to the amino acid sequence of SEQ ID NO: 327;
(17αA2-c) a protein comprising an amino acid sequence encoded by a nucleotide sequence with deletion, substitution, insertion or addition of one or several nucleotides in SEQ ID NO: 326;
(17αA2-d) a protein comprising an amino acid sequence encoded by a nucleotide sequence having at least 70% identity to the nucleotide sequence of SEQ ID NO: 326; or
(17αA2-e) a protein comprising an amino acid sequence encoded by a nucleic acid that hybridizes under stringent conditions with a nucleic acid having a nucleotide sequence complementary to the nucleotide sequence of SEQ ID NO: 326; or
(17αB2) a protein comprising at least one amino acid sequence selected from the group consisting of SEQ ID NOs: 328-332;
   (18α) (18αA) hypothetical protein GLYMA_10G028300 or a variant thereof, which is defined below in any of (18αA-a) to (18αA-e):
(18αA-a) a protein comprising an amino acid sequence with deletion, substitution, insertion or addition of one or several amino acids in SEQ ID NO: 334;
(18αA-b) a protein comprising an amino acid sequence having at least 70% identity to the amino acid sequence of SEQ ID NO: 334;
(18αA-c) a protein comprising an amino acid sequence encoded by a nucleotide sequence with deletion, substitution, insertion or addition of one or several nucleotides in SEQ ID NO: 333;
(18αA-d) a protein comprising an amino acid sequence encoded by a nucleotide sequence having at least 70% identity to the nucleotide sequence of SEQ ID NO: 333; or
(18αA-e) a protein comprising an amino acid sequence encoded by a nucleic acid that hybridizes under stringent conditions with a nucleic acid having a nucleotide sequence complementary to the nucleotide sequence of SEQ ID NO: 333; or
(18αB) a protein comprising at least one amino acid sequence selected from the group consisting of SEQ ID NOs: 335-339;
   (19α) (19αA) sucrose binding protein homolog S-64 or a variant thereof, which is defined below in any of (19αA-a) to (19αA-e):
(19αA-a) a protein comprising an amino acid sequence with deletion, substitution, insertion or addition of one or several amino acids in SEQ ID NO: 341;
(19A-b) a protein comprising an amino acid sequence having at least 70% identity to the amino acid sequence of SEQ ID NO: 341;
(19αA-c) a protein comprising an amino acid sequence encoded by a nucleotide sequence with deletion, substitution, insertion or addition of one or several nucleotides in SEQ ID NO: 340;
(19αA-d) a protein comprising an amino acid sequence encoded by a nucleotide sequence having at least 70% identity to the nucleotide sequence of SEQ ID NO: 340; or
(19αA-e) a protein comprising an amino acid sequence encoded by a nucleic acid that hybridizes under stringent conditions with a nucleic acid having a nucleotide sequence complementary to the nucleotide sequence of SEQ ID NO: 340; or
(19αB) a protein comprising at least one amino acid sequence selected from the group consisting of SEQ ID NOs: 342-346;
   (25α) (25αA1) succinyl-CoA ligase [ADP-forming] subunit beta, mitochondrial or a variant thereof, which is defined below in any of (25αA1-a) to (25αA1-e):
(25αA1-a) a protein comprising an amino acid sequence with deletion, substitution, insertion or addition of one or several amino acids in SEQ ID NO: 348;
(25αA1-b) a protein comprising an amino acid sequence having at least 70% identity to the amino acid sequence of SEQ ID NO: 348;
(25αA1-c) a protein comprising an amino acid sequence encoded by a nucleotide sequence with deletion, substitution, insertion or addition of one or several nucleotides in SEQ ID NO: 347;
(25αA1-d) a protein comprising an amino acid sequence encoded by a nucleotide sequence having at least 70% identity to the nucleotide sequence of SEQ ID NO: 347; or
(25αA1-e) a protein comprising an amino acid sequence encoded by a nucleic acid that hybridizes under stringent conditions with a nucleic acid having a nucleotide sequence complementary to the nucleotide sequence of SEQ ID NO: 347;
(25αB1) a protein comprising at least one amino acid sequence selected from the group consisting of SEQ ID NOs: 349-356;
(25αA2) succinyl-CoA ligase [ADP-forming] subunit beta, mitochondrial or a variant thereof, which is defined below in any of (25αA2-a) to (25αA2-e):
(25αA2-a) a protein comprising an amino acid sequence with deletion, substitution, insertion or addition of one or several amino acids in SEQ ID NO: 358;
(25αA2-b) a protein comprising an amino acid sequence having at least 70% identity to the amino acid sequence of SEQ ID NO: 358;
(25αA2-c) a protein comprising an amino acid sequence encoded by a nucleotide sequence with deletion, substitution, insertion or addition of one or several nucleotides in SEQ ID NO: 357;
(25αA2-d) a protein comprising an amino acid sequence encoded by a nucleotide sequence having at least 70% identity to the nucleotide sequence of SEQ ID NO: 357; or
(25αA2-e) a protein comprising an amino acid sequence encoded by a nucleic acid that hybridizes under stringent conditions with a nucleic acid having a nucleotide sequence complementary to the nucleotide sequence of SEQ ID NO: 357; or
(25αB2) a protein comprising at least one amino acid sequence selected from the group consisting of SEQ ID NOs: 359-365;
   (26α) (26αA1) phosphoglycerate kinase, cytosolic or a variant thereof, which is defined below in any of (26αA1-a) to (26αA1-e):
(26αA1-a) a protein comprising an amino acid sequence with deletion, substitution, insertion or addition of one or several amino acids in SEQ ID NO: 367;
(26αA1-b) a protein comprising an amino acid sequence having at least 70% identity to the amino acid sequence of SEQ ID NO: 367;
(26αA1-c) a protein comprising an amino acid sequence encoded by a nucleotide sequence with deletion, substitution, insertion or addition of one or several nucleotides in SEQ ID NO: 366;
(26αA1-d) a protein comprising an amino acid sequence encoded by a nucleotide sequence having at least 70% identity to the nucleotide sequence of SEQ ID NO: 366; or
(26αA1-e) a protein comprising an amino acid sequence encoded by a nucleic acid that hybridizes under stringent conditions with a nucleic acid having a nucleotide sequence complementary to the nucleotide sequence of SEQ ID NO: 366;
(26αB1) a protein comprising at least one amino acid sequence selected from the group consisting of SEQ ID NOs: 368-382;
(26αA2) phosphoglycerate kinase, cytosolic or a variant thereof, which is defined below in any of (26αA2-a) to (26αA2-e):
(26αA2-a) a protein comprising an amino acid sequence with deletion, substitution, insertion or addition of one or several amino acids in SEQ ID NO: 384;
(26αA2-b) a protein comprising an amino acid sequence having at least 70% identity to the amino acid sequence of SEQ ID NO: 384;
(26αA2-c) a protein comprising an amino acid sequence encoded by a nucleotide sequence with deletion, substitution, insertion or addition of one or several nucleotides in SEQ ID NO: 383;
(26αA2-d) a protein comprising an amino acid sequence encoded by a nucleotide sequence having at least 70% identity to the nucleotide sequence of SEQ ID NO: 383; or
(26A2-e) a protein comprising an amino acid sequence encoded by a nucleic acid that hybridizes under stringent conditions with a nucleic acid having a nucleotide sequence complementary to the nucleotide sequence of SEQ ID NO: 383;
(26αB2) a protein comprising at least one amino acid sequence selected from the group consisting of SEQ ID NOs: 385-392;
(26αA3) phosphoglycerate kinase, cytosolic or a variant thereof, which is defined below in any of (26αA3-a) to (26αA3-e):
(26αA3-a) a protein comprising an amino acid sequence with deletion, substitution, insertion or addition of one or several amino acids in SEQ ID NO: 394;
(26αA3-b) a protein comprising an amino acid sequence having at least 70% identity to the amino acid sequence of SEQ ID NO: 394;
(26αA3-c) a protein comprising an amino acid sequence encoded by a nucleotide sequence with deletion, substitution, insertion or addition of one or several nucleotides in SEQ ID NO: 393;
(26αA3-d) a protein comprising an amino acid sequence encoded by a nucleotide sequence having at least 70% identity to the nucleotide sequence of SEQ ID NO: 393; or
(26αA3-e) a protein comprising an amino acid sequence encoded by a nucleic acid that hybridizes under stringent conditions with a nucleic acid having a nucleotide sequence complementary to the nucleotide sequence of SEQ ID NO: 393; or
(26αB3) a protein comprising at least one amino acid sequence selected from the group consisting of SEQ ID NOs: 395-409;
   (27α) (27αA1) alcohol dehydrogenase 1 or a variant thereof, which is defined below in any of (27αA1-a) to (27αA1-e):
(27αA1-a) a protein comprising an amino acid sequence with deletion, substitution, insertion or addition of one or several amino acids in SEQ ID NO: 411;
(27αA1-b) a protein comprising an amino acid sequence having at least 70% identity to the amino acid sequence of SEQ ID NO: 411;
(27αA1-c) a protein comprising an amino acid sequence encoded by a nucleotide sequence with deletion, substitution, insertion or addition of one or several nucleotides in SEQ ID NO: 410;
(27αA1-d) a protein comprising an amino acid sequence encoded by a nucleotide sequence having at least 70% identity to the nucleotide sequence of SEQ ID NO: 410; or
(27αA1-e) a protein comprising an amino acid sequence encoded by a nucleic acid that hybridizes under stringent conditions with a nucleic acid having a nucleotide sequence complementary to the nucleotide sequence of SEQ ID NO: 410;
(27αB1) a protein comprising at least one amino acid sequence selected from the group consisting of SEQ ID NOs: 412-416;
(27αA2) alcohol dehydrogenase 1 or a variant thereof, which is defined below in any of (27αA2-a) to (27αA2-e):
(27αA2-a) a protein comprising an amino acid sequence with deletion, substitution, insertion or addition of one or several amino acids in SEQ ID NO: 418;
(27αA2-b) a protein comprising an amino acid sequence having at least 70% identity to the amino acid sequence of SEQ ID NO: 418;
(27αA2-c) a protein comprising an amino acid sequence encoded by a nucleotide sequence with deletion, substitution, insertion or addition of one or several nucleotides in SEQ ID NO: 417;
(27αA2-d) a protein comprising an amino acid sequence encoded by a nucleotide sequence having at least 70% identity to the nucleotide sequence of SEQ ID NO: 417; or
(27αA2-e) a protein comprising an amino acid sequence encoded by a nucleic acid that hybridizes under stringent conditions with a nucleic acid having a nucleotide sequence complementary to the nucleotide sequence of SEQ ID NO: 417;
(27αB2) a protein comprising at least one amino acid sequence selected from the group consisting of SEQ ID NOs: 419 and 420;
(27αA3) alcohol dehydrogenase 1-like or a variant thereof, which is defined below in any of (27αA3-a) to (27αA3-e):
(27αA3-a) a protein comprising an amino acid sequence with deletion, substitution, insertion or addition of one or several amino acids in SEQ ID NO: 422;
(27αA3-b) a protein comprising an amino acid sequence having at least 70% identity to the amino acid sequence of SEQ ID NO: 422;
(27αA3-c) a protein comprising an amino acid sequence encoded by a nucleotide sequence with deletion, substitution, insertion or addition of one or several nucleotides in SEQ ID NO: 421;
(27αA3-d) a protein comprising an amino acid sequence encoded by a nucleotide sequence having at least 70% identity to the nucleotide sequence of SEQ ID NO: 421; or
(27αA3-e) a protein comprising an amino acid sequence encoded by a nucleic acid that hybridizes under stringent conditions with a nucleic acid having a nucleotide sequence complementary to the nucleotide sequence of SEQ ID NO: 421; or
(27αB3) a protein comprising at least one amino acid sequence selected from the group consisting of SEQ ID NOs: 423-427;
   (28α) (28αA) 35 kDa seed maturation protein isoform X1 or a variant thereof, which is defined below in any of (28αA-a) to (28αA-e):
(28αA-a) a protein comprising an amino acid sequence with deletion, substitution, insertion or addition of one or several amino acids in SEQ ID NO: 429;
(28αA-b) a protein comprising an amino acid sequence having at least 70% identity to the amino acid sequence of SEQ ID NO: 429;
(28αA-c) a protein comprising an amino acid sequence encoded by a nucleotide sequence with deletion, substitution, insertion or addition of one or several nucleotides in SEQ ID NO: 428;
(28αA-d) a protein comprising an amino acid sequence encoded by a nucleotide sequence having at least 70% identity to the nucleotide sequence of SEQ ID NO: 428; or
(28αA-e) a protein comprising an amino acid sequence encoded by a nucleic acid that hybridizes under stringent conditions with a nucleic acid having a nucleotide sequence complementary to the nucleotide sequence of SEQ ID NO: 428; or
(28αB) a protein comprising at least one amino acid sequence selected from the group consisting of SEQ ID NOs: 430-433;
   (29-33) (29-33αA1) glyceraldehyde-3-phosphate dehydrogenase isoform X1 or a variant thereof, which is defined below in any of (29-33αA1-a) to (29-33αA1-e):
(29-33αA1-a) a protein comprising an amino acid sequence with deletion, substitution, insertion or addition of one or several amino acids in SEQ ID NO: 435;
(29-33αA1-b) a protein comprising an amino acid sequence having at least 70% identity to the amino acid sequence of SEQ ID NO: 435;
(29-33αA1-c) a protein comprising an amino acid sequence encoded by a nucleotide sequence with deletion, substitution, insertion or addition of one or several nucleotides in SEQ ID NO: 434;
(29-33αA1-d) a protein comprising an amino acid sequence encoded by a nucleotide sequence having at least 70% identity to the nucleotide sequence of SEQ ID NO: 434; or
(29-33αA1-e) a protein comprising an amino acid sequence encoded by a nucleic acid that hybridizes under stringent conditions with a nucleic acid having a nucleotide sequence complementary to the nucleotide sequence of SEQ ID NO: 434;
(29-33αB1) a protein comprising at least one amino acid sequence selected from the group consisting of SEQ ID NOs: 436-439;
(29-33αA2) glyceraldehyde-3-phosphate dehydrogenase, cytosolic or a variant thereof, which is defined below in any of (29-33αA2-a) to (29-33αA2-e):
(29-33αA2-a) a protein comprising an amino acid sequence with deletion, substitution, insertion or addition of one or several amino acids in SEQ ID NO: 441;
(29-33αA2-b) a protein comprising an amino acid sequence having at least 70% identity to the amino acid sequence of SEQ ID NO: 441;
(29-33αA2-c) a protein comprising an amino acid sequence encoded by a nucleotide sequence with deletion, substitution, insertion or addition of one or several nucleotides in SEQ ID NO: 440;
(29-33αA2-d) a protein comprising an amino acid sequence encoded by a nucleotide sequence having at least 70% identity to the nucleotide sequence of SEQ ID NO: 440; or
(29-33αA2-e) a protein comprising an amino acid sequence encoded by a nucleic acid that hybridizes under stringent conditions with a nucleic acid having a nucleotide sequence complementary to the nucleotide sequence of SEQ ID NO: 440;
(29-33αB2) a protein comprising at least one amino acid sequence selected from the group consisting of SEQ ID NOs: 442-445;
(29-33αA3) uncharacterized protein LOC100782924 or a variant thereof, which is defined below in any of (29-33αA3-a) to (29-33αA3-e):
(29-33αA3-a) a protein comprising an amino acid sequence with deletion, substitution, insertion or addition of one or several amino acids in SEQ ID NO: 447;
(29-33αA3-b) a protein comprising an amino acid sequence having at least 70% identity to the amino acid sequence of SEQ ID NO: 447;
(29-33αA3-c) a protein comprising an amino acid sequence encoded by a nucleotide sequence with deletion, substitution, insertion or addition of one or several nucleotides in SEQ ID NO: 446;
(29-33αA3-d) a protein comprising an amino acid sequence encoded by a nucleotide sequence having at least 70% identity to the nucleotide sequence of SEQ ID NO: 446; or
(29-33αA3-e) a protein comprising an amino acid sequence encoded by a nucleic acid that hybridizes under stringent conditions with a nucleic acid having a nucleotide sequence complementary to the nucleotide sequence of SEQ ID NO: 446; or
(29-33αB3) a protein comprising at least one amino acid sequence selected from the group consisting of SEQ ID NOs: 448-451,
   (34α) (34αA1) bifunctional UDP-glucose 4-epimerase and UDP-xylose 4-epimerase 1-like or a variant thereof, which is defined below in any of (34αA1-a) to (34αA1-e):
(34αA1-a) a protein comprising an amino acid sequence with deletion, substitution, insertion or addition of one or several amino acids in SEQ ID NO: 453;
(34αA1-b) a protein comprising an amino acid sequence having at least 70% identity to the amino acid sequence of SEQ ID NO: 453;
(34αA1-c) a protein comprising an amino acid sequence encoded by a nucleotide sequence with deletion, substitution, insertion or addition of one or several nucleotides in SEQ ID NO: 452;
(34αA1-d) a protein comprising an amino acid sequence encoded by a nucleotide sequence having at least 70% identity to the nucleotide sequence of SEQ ID NO: 452; or
(34αA1-e) a protein comprising an amino acid sequence encoded by a nucleic acid that hybridizes under stringent conditions with a nucleic acid having a nucleotide sequence complementary to the nucleotide sequence of SEQ ID NO: 452;
(34αB1) a protein comprising an amino acid sequence of SEQ ID NO: 454;
(34αA2) uncharacterized protein LOC100803483 or a variant thereof, which is defined below in any of (34αA2-a) to (34αA2-e):
(34αA2-a) a protein comprising an amino acid sequence with deletion, substitution, insertion or addition of one or several amino acids in SEQ ID NO: 456;
(34αA2-b) a protein comprising an amino acid sequence having at least 70% identity to the amino acid sequence of SEQ ID NO: 456;
(34αA2-c) a protein comprising an amino acid sequence encoded by a nucleotide sequence with deletion, substitution, insertion or addition of one or several nucleotides in SEQ ID NO: 455;
(34αA2-d) a protein comprising an amino acid sequence encoded by a nucleotide sequence having at least 70% identity to the nucleotide sequence of SEQ ID NO: 455; or
(34αA2-e) a protein comprising an amino acid sequence encoded by a nucleic acid that hybridizes under stringent conditions with a nucleic acid having a nucleotide sequence complementary to the nucleotide sequence of SEQ ID NO: 455; or
(34αB2) a protein comprising at least one amino acid sequence selected from the group consisting of SEQ ID NOs: 457 and 458;
   (35α) (35αA1) elongation factor 1-alpha or a variant thereof, which is defined below in any of (35αA1-a) to (35αA1-e):
(35αA1-a) a protein comprising an amino acid sequence with deletion, substitution, insertion or addition of one or several amino acids in SEQ ID NO: 460;
(35αA1-b) a protein comprising an amino acid sequence having at least 70% identity to the amino acid sequence of SEQ ID NO: 460;
(35αA1-c) a protein comprising an amino acid sequence encoded by a nucleotide sequence with deletion, substitution, insertion or addition of one or several nucleotides in SEQ ID NO: 459;
(35αA1-d) a protein comprising an amino acid sequence encoded by a nucleotide sequence having at least 70% identity to the nucleotide sequence of SEQ ID NO: 459; or
(35αA1-e) a protein comprising an amino acid sequence encoded by a nucleic acid that hybridizes under stringent conditions with a nucleic acid having a nucleotide sequence complementary to the nucleotide sequence of SEQ ID NO: 459;
(35αB1) a protein comprising at least one amino acid sequence selected from the group consisting of SEQ ID NOs: 461-466;
(35αA2) elongation factor 1-alpha or a variant thereof, which is defined below in any of (35αA2-a) to (35αA2-e):
(35αA2-a) a protein comprising an amino acid sequence with deletion, substitution, insertion or addition of one or several amino acids in SEQ ID NO: 468;
(35αA2-b) a protein comprising an amino acid sequence having at least 70% identity to the amino acid sequence of SEQ ID NO: 468;
(35αA2-c) a protein comprising an amino acid sequence encoded by a nucleotide sequence with deletion, substitution, insertion or addition of one or several nucleotides in SEQ ID NO: 467;
(35αA2-d) a protein comprising an amino acid sequence encoded by a nucleotide sequence having at least 70% identity to the nucleotide sequence of SEQ ID NO: 467; or
(35αA2-e) a protein comprising an amino acid sequence encoded by a nucleic acid that hybridizes under stringent conditions with a nucleic acid having a nucleotide sequence complementary to the nucleotide sequence of SEQ ID NO: 467;
(35αB2) a protein comprising at least one amino acid sequence selected from the group consisting of SEQ ID NOs: 469-471;
(35αA3) elongation factor-1A isoform X1 or a variant thereof, which is defined below in any of (35αA3-a) to (35αA3-e):
(35αA3-a) a protein comprising an amino acid sequence with deletion, substitution, insertion or addition of one or several amino acids in SEQ ID NO: 473;
(35αA3-b) a protein comprising an amino acid sequence having at least 70% identity to the amino acid sequence of SEQ ID NO: 473;
(35αA3-c) a protein comprising an amino acid sequence encoded by a nucleotide sequence with deletion, substitution, insertion or addition of one or several nucleotides in SEQ ID NO: 472;
(35αA3-d) a protein comprising an amino acid sequence encoded by a nucleotide sequence having at least 70% identity to the nucleotide sequence of SEQ ID NO: 472; or
(35αA3-e) a protein comprising an amino acid sequence encoded by a nucleic acid that hybridizes under stringent conditions with a nucleic acid having a nucleotide sequence complementary to the nucleotide sequence of SEQ ID NO: 472; or
(35αB3) a protein comprising at least one amino acid sequence selected from the group consisting of SEQ ID NOs: 474-477;
   (36, 37α) (36, 37αA1) seed maturation protein PM34 or a variant thereof, which is defined below in any of (36, 37αA1-a) to (36, 37αA1-e):
(36, 37αA1-a) a protein comprising an amino acid sequence with deletion, substitution, insertion or addition of one or several amino acids in SEQ ID NO: 479;
(36, 37αA1-b) a protein comprising an amino acid sequence having at least 70% identity to the amino acid sequence of SEQ ID NO: 479;
(36, 37αA1-c) a protein comprising an amino acid sequence encoded by a nucleotide sequence with deletion, substitution, insertion or addition of one or several nucleotides in SEQ ID NO: 478;
(36, 37αA1-d) a protein comprising an amino acid sequence encoded by a nucleotide sequence having at least 70% identity to the nucleotide sequence of SEQ ID NO: 478; or
(36, 37αA1-e) a protein comprising an amino acid sequence encoded by a nucleic acid that hybridizes under stringent conditions with a nucleic acid having a nucleotide sequence complementary to the nucleotide sequence of SEQ ID NO: 478;
(36, 37αB1) a protein comprising at least one amino acid sequence selected from the group consisting of SEQ ID NOs: 480-483;
(36, 37αA2) uncharacterized protein LOC100809384 or a variant thereof, which is defined below in any of (36, 37αA2-a) to (36, 37αA2-e):
(36, 37αA2-a) a protein comprising an amino acid sequence with deletion, substitution, insertion or addition of one or several amino acids in SEQ ID NO: 485;
(36, 37αA2-b) a protein comprising an amino acid sequence having at least 70% identity to the amino acid sequence of SEQ ID NO: 485;
(36, 37αA2-c) a protein comprising an amino acid sequence encoded by a nucleotide sequence with deletion, substitution, insertion or addition of one or several nucleotides in SEQ ID NO: 484;
(36, 37αA2-d) a protein comprising an amino acid sequence encoded by a nucleotide sequence having at least 70% identity to the nucleotide sequence of SEQ ID NO: 484; or
(36, 37αA2-e) a protein comprising an amino acid sequence encoded by a nucleic acid that hybridizes under stringent conditions with a nucleic acid having a nucleotide sequence complementary to the nucleotide sequence of SEQ ID NO: 484;
(36, 37αB2) a protein comprising at least one amino acid sequence selected from the group consisting of SEQ ID NOs: 486 and 487;
(36, 37αA3) hypothetical protein GLYMA_10G155300 or a variant thereof, which is defined below in any of (36, 37αA3-a) to (36, 37αA3-e):
(36, 37αA3-a) a protein comprising an amino acid sequence with deletion, substitution, insertion or addition of one or several amino acids in SEQ ID NO: 489;
(36, 37αA3-b) a protein comprising an amino acid sequence having at least 70% identity to the amino acid sequence of SEQ ID NO: 489;
(36, 37αA3-c) a protein comprising an amino acid sequence encoded by a nucleotide sequence with deletion, substitution, insertion or addition of one or several nucleotides in SEQ ID NO: 488;
(36, 37αA3-d) a protein comprising an amino acid sequence encoded by a nucleotide sequence having at least 70% identity to the nucleotide sequence of SEQ ID NO: 488; or
(36, 37αA3-e) a protein comprising an amino acid sequence encoded by a nucleic acid that hybridizes under stringent conditions with a nucleic acid having a nucleotide sequence complementary to the nucleotide sequence of SEQ ID NO: 488; or
(36, 37αB3) a protein comprising at least one amino acid sequence selected from the group consisting of SEQ ID NOs: 490-493;
   (38α) (38αA) seed biotin-containing protein SBP65-like isoform X1 or a variant thereof, which is defined below in any of (38αA-a) to (38αA-e):
(38αA-a) a protein comprising an amino acid sequence with deletion, substitution, insertion or addition of one or several amino acids in SEQ ID NO: 495;
(38αA-b) a protein comprising an amino acid sequence having at least 70% identity to the amino acid sequence of SEQ ID NO: 495;
(38αA-c) a protein comprising an amino acid sequence encoded by a nucleotide sequence with deletion, substitution, insertion or addition of one or several nucleotides in SEQ ID NO: 494;
(38αA-d) a protein comprising an amino acid sequence encoded by a nucleotide sequence having at least 70% identity to the nucleotide sequence of SEQ ID NO: 494; or
(38αA-e) a protein comprising an amino acid sequence encoded by a nucleic acid that hybridizes under stringent conditions with a nucleic acid having a nucleotide sequence complementary to the nucleotide sequence of SEQ ID NO: 494; or
(38αB) a protein comprising at least one amino acid sequence selected from the group consisting of SEQ ID NOs: 496-513;
   (39α) (39αA) ATP synthase subunit O, mitochondrial-like or a variant thereof, which is defined below in any of (39αA-a) to (39αA-e):
(39αA-a) a protein comprising an amino acid sequence with deletion, substitution, insertion or addition of one or several amino acids in SEQ ID NO: 515;
(39αA-b) a protein comprising an amino acid sequence having at least 70% identity to the amino acid sequence of SEQ ID NO: 515;
(39αA-c) a protein comprising an amino acid sequence encoded by a nucleotide sequence with deletion, substitution, insertion or addition of one or several nucleotides in SEQ ID NO: 514;
(39αA-d) a protein comprising an amino acid sequence encoded by a nucleotide sequence having at least 70% identity to the nucleotide sequence of SEQ ID NO: 514; or
(39αA-e) a protein comprising an amino acid sequence encoded by a nucleic acid that hybridizes under stringent conditions with a nucleic acid having a nucleotide sequence complementary to the nucleotide sequence of SEQ ID NO: 514; or
(39αB) a protein comprising an amino acid sequence of SEQ ID NO: 516;
   (40, 56α) (40, 56αA) P24 oleosin isoform A or a variant thereof, which is defined below in any of (40, 56αA-a) to (40, 56αA-e):
(40, 56αA-a) a protein comprising an amino acid sequence with deletion, substitution, insertion or addition of one or several amino acids in SEQ ID NO: 518;
(40, 56αA-b) a protein comprising an amino acid sequence having at least 70% identity to the amino acid sequence of SEQ ID NO: 518;
(40, 56αA-c) a protein comprising an amino acid sequence encoded by a nucleotide sequence with deletion, substitution, insertion or addition of one or several nucleotides in SEQ ID NO: 517;
(40, 56αA-d) a protein comprising an amino acid sequence encoded by a nucleotide sequence having at least 70% identity to the nucleotide sequence of SEQ ID NO: 517; or
(40, 56αA-e) a protein comprising an amino acid sequence encoded by a nucleic acid that hybridizes under stringent conditions with a nucleic acid having a nucleotide sequence complementary to the nucleotide sequence of SEQ ID NO: 517; or
(40, 56αB) a protein comprising at least one amino acid sequence selected from the group consisting of SEQ ID NOs: 519 and 520;
   (41α) (41αA) uncharacterized protein At3g49720-like isoform X2 or a variant thereof, which is defined below in any of (41αA-a) to (41αA-e):
(41αA-a) a protein comprising an amino acid sequence with deletion, substitution, insertion or addition of one or several amino acids in SEQ ID NO: 522;
(41αA-b) a protein comprising an amino acid sequence having at least 70% identity to the amino acid sequence of SEQ ID NO: 522;
(41αA-c) a protein comprising an amino acid sequence encoded by a nucleotide sequence with deletion, substitution, insertion or addition of one or several nucleotides in SEQ ID NO: 521;
(41αA-d) a protein comprising an amino acid sequence encoded by a nucleotide sequence having at least 70% identity to the nucleotide sequence of SEQ ID NO: 521; or
(41αA-e) a protein comprising an amino acid sequence encoded by a nucleic acid that hybridizes under stringent conditions with a nucleic acid having a nucleotide sequence complementary to the nucleotide sequence of SEQ ID NO: 521; or
(41αB) a protein comprising an amino acid sequence of SEQ ID NO: 523; (42, 43α) (42, 43αA1) superoxide dismutase [Mn], mitochondrial or a variant thereof, which is defined below in any of (42, 43αA1-a) to (42, 43αA1-e):
(42, 43αA1-a) a protein comprising an amino acid sequence with deletion, substitution, insertion or addition of one or several amino acids in SEQ ID NO: 525;
(42, 43αA1-b) a protein comprising an amino acid sequence having at least 70% identity to the amino acid sequence of SEQ ID NO: 525;
(42, 43αA1-c) a protein comprising an amino acid sequence encoded by a nucleotide sequence with deletion, substitution, insertion or addition of one or several nucleotides in SEQ ID NO: 524;
(42, 43αA1-d) a protein comprising an amino acid sequence encoded by a nucleotide sequence having at least 70% identity to the nucleotide sequence of SEQ ID NO: 524; or
(42, 43αA1-e) a protein comprising an amino acid sequence encoded by a nucleic acid that hybridizes under stringent conditions with a nucleic acid having a nucleotide sequence complementary to the nucleotide sequence of SEQ ID NO: 524;
(42, 43αB1) a protein comprising at least one amino acid sequence selected from the group consisting of SEQ ID NOs: 526-531;
(42, 43αA2) hypothetical protein GLYMA_04G221300 or a variant thereof, which is defined below in any of (42, 43αA2-a) to (42, 43αA2-e):
(42, 43αA2-a) a protein comprising an amino acid sequence with deletion, substitution, insertion or addition of one or several amino acids in SEQ ID NO: 533;
(42, 43αA2-b) a protein comprising an amino acid sequence having at least 70% identity to the amino acid sequence of SEQ ID NO: 533;
(42, 43αA2-c) a protein comprising an amino acid sequence encoded by a nucleotide sequence with deletion, substitution, insertion or addition of one or several nucleotides in SEQ ID NO: 532;
(42, 43αA2-d) a protein comprising an amino acid sequence encoded by a nucleotide sequence having at least 70% identity to the nucleotide sequence of SEQ ID NO: 532; or
(42, 43αA2-e) a protein comprising an amino acid sequence encoded by a nucleic acid that hybridizes under stringent conditions with a nucleic acid having a nucleotide sequence complementary to the nucleotide sequence of SEQ ID NO: 532; or
(42, 43αB2) a protein comprising at least one amino acid sequence selected from the group consisting of SEQ ID NOs: 534-539;
   (45α) (45αA1) uncharacterized protein LOC100499771 or a variant thereof, which is defined below in any of (45αA1-a) to (45αA1-e):
(45αA1-a) a protein comprising an amino acid sequence with deletion, substitution, insertion or addition of one or several amino acids in SEQ ID NO: 541;
(45αA1-b) a protein comprising an amino acid sequence having at least 70% identity to the amino acid sequence of SEQ ID NO: 541;
(45αA1-c) a protein comprising an amino acid sequence encoded by a nucleotide sequence with deletion, substitution, insertion or addition of one or several nucleotides in SEQ ID NO: 540;
(45αA1-d) a protein comprising an amino acid sequence encoded by a nucleotide sequence having at least 70% identity to the nucleotide sequence of SEQ ID NO: 540; or
(45αA1-e) a protein comprising an amino acid sequence encoded by a nucleic acid that hybridizes under stringent conditions with a nucleic acid having a nucleotide sequence complementary to the nucleotide sequence of SEQ ID NO: 540;
(45αB1) a protein comprising at least one amino acid sequence selected from the group consisting of SEQ ID NOs: 542-545;
(45αA2) hypothetical protein GLYMA_09G192800 or a variant thereof, which is defined below in any of (45αA2-a) to (45αA2-e):
(45αA2-a) a protein comprising an amino acid sequence with deletion, substitution, insertion or addition of one or several amino acids in SEQ ID NO: 547;
(45αA2-b) a protein comprising an amino acid sequence having at least 70% identity to the amino acid sequence of SEQ ID NO: 547;
(45αA2-c) a protein comprising an amino acid sequence encoded by a nucleotide sequence with deletion, substitution, insertion or addition of one or several nucleotides in SEQ ID NO: 546;
(45αA2-d) a protein comprising an amino acid sequence encoded by a nucleotide sequence having at least 70% identity to the nucleotide sequence of SEQ ID NO: 546; or
(45αA2-e) a protein comprising an amino acid sequence encoded by a nucleic acid that hybridizes under stringent conditions with a nucleic acid having a nucleotide sequence complementary to the nucleotide sequence of SEQ ID NO: 546; or
(45αB2) a protein comprising at least one amino acid sequence selected from the group consisting of SEQ ID NOs: 548-551;
   (46α) (46αA) seed maturation protein PM22 or a variant thereof, which is defined below in any of (46αA-a) to (46αA-e):
(46αA-a) a protein comprising an amino acid sequence with deletion, substitution, insertion or addition of one or several amino acids in SEQ ID NO: 553;
(46αA-b) a protein comprising an amino acid sequence having at least 70% identity to the amino acid sequence of SEQ ID NO: 553;
(46αA-c) a protein comprising an amino acid sequence encoded by a nucleotide sequence with deletion, substitution, insertion or addition of one or several nucleotides in SEQ ID NO: 552;
(46αA-d) a protein comprising an amino acid sequence encoded by a nucleotide sequence having at least 70% identity to the nucleotide sequence of SEQ ID NO: 552; or
(46αA-e) a protein comprising an amino acid sequence encoded by a nucleic acid that hybridizes under stringent conditions with a nucleic acid having a nucleotide sequence complementary to the nucleotide sequence of SEQ ID NO: 552; or
(46αB) a protein comprising an amino acid sequence of SEQ ID NO: 554;
   (47α) (47αA1) eukaryotic translation initiation factor 5A-2 or a variant thereof, which is defined below in any of (47αA1-a) to (47αA1-e):
(47αA1-a) a protein comprising an amino acid sequence with deletion, substitution, insertion or addition of one or several amino acids in SEQ ID NO: 556;
(47αA1-b) a protein comprising an amino acid sequence having at least 70% identity to the amino acid sequence of SEQ ID NO: 556;
(47αA1-c) a protein comprising an amino acid sequence encoded by a nucleotide sequence with deletion, substitution, insertion or addition of one or several nucleotides in SEQ ID NO: 555;
(47αA1-d) a protein comprising an amino acid sequence encoded by a nucleotide sequence having at least 70% identity to the nucleotide sequence of SEQ ID NO: 555; or
(47αA1-e) a protein comprising an amino acid sequence encoded by a nucleic acid that hybridizes under stringent conditions with a nucleic acid having a nucleotide sequence complementary to the nucleotide sequence of SEQ ID NO: 555;
(47αB1) a protein comprising at least one amino acid sequence selected from the group consisting of SEQ ID NOs: 557 and 558;
(47αA2) uncharacterized protein LOC100305510 or a variant thereof, which is defined below in any of (47αA2-a) to (47αA2-e):
(47αA2-a) a protein comprising an amino acid sequence with deletion, substitution, insertion or addition of one or several amino acids in SEQ ID NO: 560;
(47αA2-b) a protein comprising an amino acid sequence having at least 70% identity to the amino acid sequence of SEQ ID NO: 560;
(47αA2-c) a protein comprising an amino acid sequence encoded by a nucleotide sequence with deletion, substitution, insertion or addition of one or several nucleotides in SEQ ID NO: 559;
(47αA2-d) a protein comprising an amino acid sequence encoded by a nucleotide sequence having at least 70% identity to the nucleotide sequence of SEQ ID NO: 559; or
(47αA2-e) a protein comprising an amino acid sequence encoded by a nucleic acid that hybridizes under stringent conditions with a nucleic acid having a nucleotide sequence complementary to the nucleotide sequence of SEQ ID NO: 559; or
(47αB2) a protein comprising at least one amino acid sequence selected from the group consisting of SEQ ID NOs: 561-563;
   (48α) (48αA) uncharacterized protein LOC100306570 or a variant thereof, which is defined below in any of (48αA-a) to (48αA-e):
(48αA-a) a protein comprising an amino acid sequence with deletion, substitution, insertion or addition of one or several amino acids in SEQ ID NO: 565;
(48αA-b) a protein comprising an amino acid sequence having at least 70% identity to the amino acid sequence of SEQ ID NO: 565;
(48αA-c) a protein comprising an amino acid sequence encoded by a nucleotide sequence with deletion, substitution, insertion or addition of one or several nucleotides in SEQ ID NO: 564;
(48αA-d) a protein comprising an amino acid sequence encoded by a nucleotide sequence having at least 70% identity to the nucleotide sequence of SEQ ID NO: 564; or
(48αA-e) a protein comprising an amino acid sequence encoded by a nucleic acid that hybridizes under stringent conditions with a nucleic acid having a nucleotide sequence complementary to the nucleotide sequence of SEQ ID NO: 564; or
(48αB) a protein comprising at least one amino acid sequence selected from the group consisting of SEQ ID NOs: 566-570;
   (50) (50αA) uncharacterized protein LOC100813859 or a variant thereof, which is defined below in any of (50αA-a) to (50αA-e):
(50αA-a) a protein comprising an amino acid sequence with deletion, substitution, insertion or addition of one or several amino acids in SEQ ID NO: 572;
(50αA-b) a protein comprising an amino acid sequence having at least 70% identity to the amino acid sequence of SEQ ID NO: 572;
(50αA-c) a protein comprising an amino acid sequence encoded by a nucleotide sequence with deletion, substitution, insertion or addition of one or several nucleotides in SEQ ID NO: 571;
(50αA-d) a protein comprising an amino acid sequence encoded by a nucleotide sequence having at least 70% identity to the nucleotide sequence of SEQ ID NO: 571; or
(50αA-e) a protein comprising an amino acid sequence encoded by a nucleic acid that hybridizes under stringent conditions with a nucleic acid having a nucleotide sequence complementary to the nucleotide sequence of SEQ ID NO: 571; or
(50αB) a protein comprising at least one amino acid sequence selected from the group consisting of SEQ ID NOs: 573-576;
   (51α) (51αA) 14-3-3-like protein or a variant thereof, which is defined below in any of (51αA-a) to (51αA-e):
(51αA-a) a protein comprising an amino acid sequence with deletion, substitution, insertion or addition of one or several amino acids in SEQ ID NO: 578;
(51αA-b) a protein comprising an amino acid sequence having at least 70% identity to the amino acid sequence of SEQ ID NO: 578;
(51αA-c) a protein comprising an amino acid sequence encoded by a nucleotide sequence with deletion, substitution, insertion or addition of one or several nucleotides in SEQ ID NO: 577;
(51αA-d) a protein comprising an amino acid sequence encoded by a nucleotide sequence having at least 70% identity to the nucleotide sequence of SEQ ID NO: 577; or
(51αA-e) a protein comprising an amino acid sequence encoded by a nucleic acid that hybridizes under stringent conditions with a nucleic acid having a nucleotide sequence complementary to the nucleotide sequence of SEQ ID NO: 577; or
(51αB) a protein comprising at least one amino acid sequence selected from the group consisting of SEQ ID NOs: 579-582;
   (52α) (52αA) probable fructokinase-4 or a variant thereof, which is defined below in any of (52αA-a) to (52αA-e):
(52αA-a) a protein comprising an amino acid sequence with deletion, substitution, insertion or addition of one or several amino acids in SEQ ID NO: 584;
(52αA-b) a protein comprising an amino acid sequence having at least 70% identity to the amino acid sequence of SEQ ID NO: 584;
(52αA-c) a protein comprising an amino acid sequence encoded by a nucleotide sequence with deletion, substitution, insertion or addition of one or several nucleotides in SEQ ID NO: 583;
(52αA-d) a protein comprising an amino acid sequence encoded by a nucleotide sequence having at least 70% identity to the nucleotide sequence of SEQ ID NO: 583; or
(52αA-e) a protein comprising an amino acid sequence encoded by a nucleic acid that hybridizes under stringent conditions with a nucleic acid having a nucleotide sequence complementary to the nucleotide sequence of SEQ ID NO: 583; or
(52αB) a protein comprising at least one amino acid sequence selected from the group consisting of SEQ ID NOs: 585-588;
   (53α) (53αA1) triosephosphate isomerase isoform X1 or a variant thereof, which is defined below in any of (53αA1-a) to (53αA1-e):
(53αA1-a) a protein comprising an amino acid sequence with deletion, substitution, insertion or addition of one or several amino acids in SEQ ID NO: 590;
(53αA1-b) a protein comprising an amino acid sequence having at least 70% identity to the amino acid sequence of SEQ ID NO: 590;
(53αA1-c) a protein comprising an amino acid sequence encoded by a nucleotide sequence with deletion, substitution, insertion or addition of one or several nucleotides in SEQ ID NO: 589;
(53αA1-d) a protein comprising an amino acid sequence encoded by a nucleotide sequence having at least 70% identity to the nucleotide sequence of SEQ ID NO: 589; or
(53αA1-e) a protein comprising an amino acid sequence encoded by a nucleic acid that hybridizes under stringent conditions with a nucleic acid having a nucleotide sequence complementary to the nucleotide sequence of SEQ ID NO: 589;
(53αB1) a protein comprising at least one amino acid sequence selected from the group consisting of SEQ ID NOs: 591-597;
(53αA2) triosephosphate isomerase, cytosolic or a variant thereof, which is defined below in any of (53αA2-a) to (53αA2-e):
(53αA2-a) a protein comprising an amino acid sequence with deletion, substitution, insertion or addition of one or several amino acids in SEQ ID NO: 599;
(53αA2-b) a protein comprising an amino acid sequence having at least 70% identity to the amino acid sequence of SEQ ID NO: 599;
(53αA2-c) a protein comprising an amino acid sequence encoded by a nucleotide sequence with deletion, substitution, insertion or addition of one or several nucleotides in SEQ ID NO: 598;
(53αA2-d) a protein comprising an amino acid sequence encoded by a nucleotide sequence having at least 70% identity to the nucleotide sequence of SEQ ID NO: 598; or
(53αA2-e) a protein comprising an amino acid sequence encoded by a nucleic acid that hybridizes under stringent conditions with a nucleic acid having a nucleotide sequence complementary to the nucleotide sequence of SEQ ID NO: 598; or
(53αB2) a protein comprising at least one amino acid sequence selected from the group consisting of SEQ ID NOs: 600-604;
   (54α) (54αA1) superoxide dismutase [Fe], chloroplastic isoform X1 or a variant thereof, which is defined below in any of (54αA1-a) to (54αA1-e):
(54αA1-a) a protein comprising an amino acid sequence with deletion, substitution, insertion or addition of one or several amino acids in SEQ ID NO: 606;
(54αA1-b) a protein comprising an amino acid sequence having at least 70% identity to the amino acid sequence of SEQ ID NO: 606;
(53αA1-c) a protein comprising an amino acid sequence encoded by a nucleotide sequence with deletion, substitution, insertion or addition of one or several nucleotides in SEQ ID NO: 605;
(54αA1-d) a protein comprising an amino acid sequence encoded by a nucleotide sequence having at least 70% identity to the nucleotide sequence of SEQ ID NO: 605; or
(54αA1-e) a protein comprising an amino acid sequence encoded by a nucleic acid that hybridizes under stringent conditions with a nucleic acid having a nucleotide sequence complementary to the nucleotide sequence of SEQ ID NO: 605;
(54αB1) a protein comprising at least one amino acid sequence selected from the group consisting of SEQ ID NOs: 607-611;
(54αA2) iron-superoxide dismutase or a variant thereof, which is defined below in any of (54αA2-a) to (54αA2-e):
(54αA2-a) a protein comprising an amino acid sequence with deletion, substitution, insertion or addition of one or several amino acids in SEQ ID NO: 613;
(54αA2-b) a protein comprising an amino acid sequence having at least 70% identity to the amino acid sequence of SEQ ID NO: 613;
(54αA2-c) a protein comprising an amino acid sequence encoded by a nucleotide sequence with deletion, substitution, insertion or addition of one or several nucleotides in SEQ ID NO: 612;
(54αA2-d) a protein comprising an amino acid sequence encoded by a nucleotide sequence having at least 70% identity to the nucleotide sequence of SEQ ID NO: 612; or
(54αA2-e) a protein comprising an amino acid sequence encoded by a nucleic acid that hybridizes under stringent conditions with a nucleic acid having a nucleotide sequence complementary to the nucleotide sequence of SEQ ID NO: 612; or
(54αB2) a protein comprising at least one amino acid sequence selected from the group consisting of SEQ ID NOs: 614-616; or
   (55α) (55αA1) 51 kDa seed maturation protein precursor or a variant thereof, which is defined below in any of (55αA1-a) to (55αA1-e):
(55αA1-a) a protein comprising an amino acid sequence with deletion, substitution, insertion or addition of one or several amino acids in SEQ ID NO: 618;
(55αA1-b) a protein comprising an amino acid sequence having at least 70% identity to the amino acid sequence of SEQ ID NO: 618;
(55αA1-c) a protein comprising an amino acid sequence encoded by a nucleotide sequence with deletion, substitution, insertion or addition of one or several nucleotides in SEQ ID NO: 617;
(55αA1-d) a protein comprising an amino acid sequence encoded by a nucleotide sequence having at least 70% identity to the nucleotide sequence of SEQ ID NO: 617; or
(55αA1-e) a protein comprising an amino acid sequence encoded by a nucleic acid that hybridizes under stringent conditions with a nucleic acid having a nucleotide sequence complementary to the nucleotide sequence of SEQ ID NO: 617;
(55αB1) a protein comprising at least one amino acid sequence selected from the group consisting of SEQ ID NOs: 619-635;
(55αA2) Lea protein precursor or a variant thereof, which is defined below in any of (55αA2-a) to (55αA2-e):
(55αA2-a) a protein comprising an amino acid sequence with deletion, substitution, insertion or addition of one or several amino acids in SEQ ID NO: 637;
(55αA2-b) a protein comprising an amino acid sequence having at least 70% identity to the amino acid sequence of SEQ ID NO: 637;
(55αA2-c) a protein comprising an amino acid sequence encoded by a nucleotide sequence with deletion, substitution, insertion or addition of one or several nucleotides in SEQ ID NO: 636;
(55αA2-d) a protein comprising an amino acid sequence encoded by a nucleotide sequence having at least 70% identity to the nucleotide sequence of SEQ ID NO: 636; or
(55αA2-e) a protein comprising an amino acid sequence encoded by a nucleic acid that hybridizes under stringent conditions with a nucleic acid having a nucleotide sequence complementary to the nucleotide sequence of SEQ ID NO: 636; or
(55αB2) a protein comprising at least one amino acid sequence selected from the group consisting of SEQ ID NOs: 638-649.

[2] A composition for diagnosing an allergy to a soybean, comprising, as an antigen, at least one of proteins as defined above in any of (1α) to (55α) of [1].

[3] A method for providing an indicator for diagnosing an allergy to a soybean in a subject, the method comprising the steps of:
(i) contacting a sample obtained from the subject with an antigen, wherein the sample is a solution comprising an Ig antibody;
(ii) detecting binding between the IgE antibody present in the sample from the subject and the antigen; and
(iii) when the binding between the IgE antibody in the subject and the antigen is detected, an indicator of the fact that the subject is allergic to a soybean is provided;
wherein the antigen is at least one of proteins as defined above in any of (1α) to (55α) of [1].

[4] A pharmaceutical composition comprising at least one of proteins as defined above in any of (1α) to (55α) of [1].

[5] The pharmaceutical composition as set forth in [4], wherein the pharmaceutical composition is intended for the treatment of an allergy to a soybean.

[6] A soybean or processed products of soybean in which an antigen is eliminated or reduced, wherein the antigen is at least one of proteins as defined above in any of (1α) to (55α) of [1].

[7] A method for producing processed products of soybean in which an antigen is eliminated or reduced, the method comprising the step of confirming that the antigen is eliminated or reduced in a production process of the processed products, wherein the antigen is at least one of proteins as defined above in any of (1α) to (55α) of [1].

[8] A tester for determining the presence or absence of a soybean antigen in an object of interest, comprising an antibody that binds to at least one of proteins as defined above in any of (1α) to (55α) of [1].

[9] A soybean-derived antigen which is at least one of proteins as defined above in any of (1α) to (55α) of [1] and is causative of an allergy to soybean.

The present inventors also succeeded in finding epitopes as to the novel antigens.

As mentioned above, interaction between IgE antibodies from allergic patients and particular antigens elicit allergic reactions. These IgE antibodies recognize and bind to epitopes that are particular amino acid sequences in allergen components. Since the epitopes have a relatively short amino acid sequence, the IgE antibodies are capable of binding to different allergen components if the same amino acid sequence is present in the different allergen components. When different allergen components have a common epitope so that IgE antibodies from allergic patients bind to both of them, the allergens have cross-reactivity (or also called cross-antigenicity). Thus, the epitopes defined in the present invention enable diagnosis or treatment of an allergy including cross-reactivity, and detection of a plurality of allergen components comprising the epitopes, etc.

The present invention has been completed based on this finding. Thus, in another embodiment, the present invention can be as defined below.

[10] A polypeptide specifically binding to an IgE antibody from an allergic patient, the polypeptide being any one of the following:
(1β) a polypeptide comprising at least one amino acid sequence selected from the group consisting of SEQ ID NOs: 652-660 or the sequences XXKXEXXX and XTKXEXXX;
(2) a polypeptide comprising at least one amino acid sequence selected from the group consisting of SEQ ID NOs: 662-666, 668-678 and 680-692 or the sequences DXXDKXXXX, XPDKX and XXXXDXXXXEXXXDX;
(4β) a polypeptide comprising at least one amino acid sequence selected from the group consisting of SEQ ID NOs: 718-731 or the sequence XXXXXXXKED;
(5β) a polypeptide comprising at least one amino acid sequence selected from the group consisting of SEQ ID NOs: 732-738;
(6) a polypeptide comprising at least one amino acid sequence selected from the group consisting of SEQ ID NOs: 739 and 740;
(7β) a polypeptide comprising at least one amino acid sequence selected from the group consisting of SEQ ID NOs: 741-744;
(9) a polypeptide comprising at least one amino acid sequence selected from the group consisting of SEQ ID NOs: 746-749 or the sequence XXEXXXXKDX;
(11β) a polypeptide comprising at least one amino acid sequence selected from the group consisting of SEQ ID NOs: 751-754;
(12) a polypeptide comprising the amino acid sequence of SEQ ID NO: 755;
(13β) a polypeptide comprising at least one amino acid sequence selected from the group consisting of SEQ ID NOs: 757-758 or the sequence XXGXXXXXXXXXXXE;
(14β) a polypeptide comprising at least one amino acid sequence selected from the group consisting of SEQ ID NOs: 759-764;
(15β) a polypeptide comprising at least one amino acid sequence selected from the group consisting of SEQ ID NO: 766 or the sequence XGKXXXXXXXXXXXX;
(17β) a polypeptide comprising the amino acid sequence of SEQ ID NO: 767;
(18β) a polypeptide comprising at least one amino acid sequence selected from the group consisting of SEQ ID NOs: 750 and 768-775;
(19β) a polypeptide comprising at least one amino acid sequence selected from the group consisting of SEQ ID NOs: 776-778, 780-781, 783-784 and 786-787 or the sequences XXXXXXXXRXRRXXX, XXXXXXXXXXEEEXX and XXGKXXXXXXXXXXX;
(25β) a polypeptide comprising at least one amino acid sequence selected from the group consisting of SEQ ID NOs: 814 and 815;
(26β) a polypeptide comprising at least one amino acid sequence selected from the group consisting of SEQ ID NOs: 816 and 818-819 or the sequence FDKXXXX;
(27β) a polypeptide comprising the amino acid sequence of SEQ ID NO: 820;
(28) a polypeptide comprising at least one amino acid sequence selected from the group consisting of SEQ ID NOs: 821-823 and 825-835 or the sequence XKXXXXEKXXXXXXX;
(29β) a polypeptide comprising at least one amino acid sequence selected from the group consisting of SEQ ID NOs: 837-839 or the sequence XXXXXXXDKP;
(34β) a polypeptide comprising at least one amino acid sequence selected from the group consisting of SEQ ID NOs: 841-846 or the sequence XXXXXXXXXXXDDXE;
(35β) a polypeptide comprising at least one amino acid sequence selected from the group consisting of SEQ ID NOs: 847-853 and 855-856 or the sequence XXEXLDXXXX;
(36) a polypeptide comprising at least one amino acid sequence selected from the group consisting of SEQ ID NOs: 857-861;
(38β) a polypeptide comprising at least one amino acid sequence selected from the group consisting of SEQ ID NOs: 864, 866-867, 869 and 871-892 or the sequences RGKXXXX, RGKXXXX, XRGKXXXXXX, RXKXXXXXXXXXXXE and XXXXXXXRRXXXXXR;
(39β) a polypeptide comprising at least one amino acid sequence selected from the group consisting of SEQ ID NOs: 893-895;
(40β) a polypeptide comprising at least one amino acid sequence selected from the group consisting of SEQ ID NOs: 896-899 and 901-902 or the sequence XXXXXVTAX;
(41β) a polypeptide comprising the amino acid sequence of SEQ ID NO: 903;
(42β) a polypeptide comprising at least one amino acid sequence selected from the group consisting of SEQ ID NOs: 904-906;
(45β) a polypeptide comprising at least one amino acid sequence selected from the group consisting of SEQ ID NOs: 910-912;
(46) a polypeptide comprising at least one amino acid sequence selected from the group consisting of SEQ ID NOs: 914-915 or the sequence XDXDXXXDX;
(47β) a polypeptide comprising at least one amino acid sequence selected from the group consisting of SEQ ID NOs: 916-918;
(48) a polypeptide comprising at least one amino acid sequence selected from the group consisting of SEQ ID NOs: 920-922 or the sequence XXXXXXFDK;
(50) a polypeptide comprising at least one amino acid sequence selected from the group consisting of SEQ ID NOs: 924 and 927 or the sequences XXXXKQXXX and XXDXKQXXX;
(51) a polypeptide comprising at least one amino acid sequence selected from the group consisting of SEQ ID NOs: 928-930;
(52β) a polypeptide comprising at least one amino acid sequence selected from the group consisting of SEQ ID NOs: 932-938 or the sequence XXXXXXSXXEXLXXX;
(53β) a polypeptide comprising the amino acid sequence of SEQ ID NO: 939;
(54β) a polypeptide comprising at least one amino acid sequence selected from the group consisting of SEQ ID NOs: 940 and 943 or the sequences KXXXXTX and KXXXXTQ; and
(55β) a polypeptide comprising at least one amino acid sequence selected from the group consisting of SEQ ID NOs: 944-949 and 951-952 or the sequence KDXXXXXXQ.

[11] A polypeptide specifically binding to an IgE antibody from an allergic patient, the polypeptide being any one of the following:
(3β) a polypeptide comprising at least one amino acid sequence selected from the group consisting of SEQ ID NOs: 694-695, 697 and 699-716 or the sequences XXXXXXXXXXRLXEX, XXXXXXXXXXXXXDE and XXXXGDR;
(20β) a polypeptide comprising at least one amino acid sequence selected from the group consisting of SEQ ID NOs: 788-792;
(21) a polypeptide comprising at least one amino acid sequence selected from the group consisting of SEQ ID NOs: 793-802;
(22β) a polypeptide comprising at least one amino acid sequence selected from the group consisting of SEQ ID NOs: 803-808;
(23β) a polypeptide comprising at least one amino acid sequence selected from the group consisting of SEQ ID NOs: 809-812;
(24β) a polypeptide comprising the amino acid sequence of SEQ ID NO: 813;
(44β) a polypeptide comprising at least one amino acid sequence selected from the group consisting of SEQ ID NOs: 907-909;
(49) a polypeptide comprising the amino acid sequence of SEQ ID NO: 923;
(57) a polypeptide comprising at least one amino acid sequence selected from the group consisting of SEQ ID NOs: 953-955;
(58β) a polypeptide comprising at least one amino acid sequence selected from the group consisting of SEQ ID NOs: 956-960;
(59β) a polypeptide comprising at least one amino acid sequence selected from the group consisting of SEQ ID NOs: 961 and 962; and
(60β) a polypeptide comprising the amino acid sequence of SEQ ID NO: 963.

[12] The polypeptide according to the above or [11], wherein the number of amino acid residues is 500 or less.

[13] A kit for diagnosing an allergy, comprising at least one of polypeptides according to any one of the above to [12].

[14] A composition for diagnosing an allergy, the composition comprising at least one of polypeptides according to any one of the above to as an antigen.

[15] A method for providing an indicator for diagnosing an allergy in a subject, the method comprising the steps of:
(i) contacting a sample obtained from the subject with an antigen, wherein the sample is a solution comprising an Ig antibody;
(ii) detecting binding between the IgE antibody present in the sample obtained from the subject and the antigen; and
(iii) when the binding between the IgE antibody in the subject and the antigen is detected, an indicator of the fact that the subject is allergic is provided;
wherein the antigen is at least one of polypeptides according to any one of the above to [12].

[16] A pharmaceutical composition comprising at least one of polypeptides according to any one of the above to [12].

[17] The pharmaceutical composition according to the above [16], wherein the pharmaceutical composition is intended for the treatment of an allergy.

[18] A tester for determining the presence or absence of an antigen in an object of interest, the tester comprising an antibody that binds to at least one of polypeptides according to any one of the above to [12].

[19] A food raw material or an edible processed product in which an antigen is eliminated or reduced, or a polypeptide is cleaved or removed, wherein the antigen is at least one of polypeptides according to any one of the above to [12].

[20] A method for producing an edible processed product in which an antigen is eliminated or reduced, or a polypeptide is cleaved or removed, the method comprising the step of confirming that the antigen is eliminated or reduced, or the polypeptide is cleaved or removed, in a production process of the processed product, wherein the antigen is at least one of polypeptides according to any one of the above to [12].

### Advantageous Effects of Invention

The present invention can provide novel antigens of an allergy to soybean. Since the novel antigens (allergen components) that trigger a soybean allergy were identified according to this invention, this invention can provide highly sensitive methods and kits for diagnosing an allergy to soybean, pharmaceutical compositions comprising such an antigen, soybean or processed products of soybean in which such an antigen is eliminated or reduced.

The present invention can provide novel polypeptides comprising an epitope of an allergen. Use of the polypeptide of the present invention enables provision of highly sensitive methods and kits for diagnosing an allergy, comprising such a polypeptide, pharmaceutical compositions comprising such a polypeptide, food raw materials or edible processed products in which an antigen comprising such a polypeptide is eliminated or reduced, or such a polypeptide is cleaved or removed, and a method for producing the edible processed products.

### BRIEF DESCRIPTION OF DRAWINGS

FIG. 1 is a photograph of a gel showing a protein electrophoretic pattern in two-dimensional electrophoresis of proteins contained in soybean. The bands at the left of the photograph are bands of molecular weight markers.

FIG. 2 is a photograph of an immunoblot of a two-dimensional electrophoretic pattern of proteins contained in soybean stained with serum of a healthy subject.

FIG. 3A is a photograph of an immunoblot of a two-dimensional electrophoretic pattern of proteins contained in soybean stained with serum of Soybean-allergic patient 1α. The spots indicated with a white line or enclosed in a square are spot where the serum of the soybean-allergic patient specifically reacted. The numbers (n) indicated are the numbers of the spots and correspond to the spots indicated with “nα” herein.

FIG. 3B is a photograph of an immunoblot of a two-dimensional electrophoretic pattern of proteins contained in soybean stained with serum of Soybean-allergic patient 2α. The spots indicated with a white line or enclosed in a square are spot where the serum of the soybean-allergic patient specifically reacted. The numbers (n) indicated are the numbers of the spots and correspond to the spots indicated with “nα” herein.

FIG. 3C is a photograph of an immunoblot of a two-dimensional electrophoretic pattern of proteins contained in soybean stained with serum of Soybean-allergic patient 3α. The spots indicated with a white line or enclosed in a square are spot where the serum of the soybean-allergic patient specifically reacted. The numbers (n) indicated are the numbers of the spots and correspond to the spots indicated with “nα” herein.

FIG. 3D is a photograph of an immunoblot of a two-dimensional electrophoretic pattern of proteins contained in soybean stained with serum of Soybean-allergic patient 4α. The spots indicated with a while line are spots where the serum of the soybean-allergic patient specifically reacted. The numbers (n) indicated are the numbers of the spots and correspond to the spots indicated with “nα” herein.

FIG. 3E is a photograph of an immunoblot of a two-dimensional electrophoretic pattern of proteins contained in soybean stained with serum of Soybean-allergic patient 5α. The spots indicated with a while line are spots where the serum of the soybean-allergic patient specifically reacted. The numbers (n) indicated are the numbers of the spots and correspond to the spots indicated with “nα” herein.

FIG. 3F is a photograph of an immunoblot of a two-dimensional electrophoretic pattern of proteins contained in soybean stained with serum of Soybean-allergic patient 6α. The spots indicated with a white line or enclosed in a square are spot where the serum of the soybean-allergic patient specifically reacted. The numbers (n) indicated are the numbers of the spots and correspond to the spots indicated with “nα” herein.

FIG. 3G is a photograph of an immunoblot of a two-dimensional electrophoretic pattern of proteins contained in soybean stained with serum of Soybean-allergic patient 7α. The spots enclosed in a square are spots where the serum of the soybean-allergic patient specifically reacted. The numbers (n) indicated are the numbers of the spots and correspond to the spots indicated with “nα” herein.

FIG. 3H is a photograph of an immunoblot of a two-dimensional electrophoretic pattern of proteins contained in soybean stained with serum of Soybean-allergic patient 8α. The spots indicated with a white line or enclosed in a square are spot where the serum of the soybean-allergic patient specifically reacted. The numbers (n) indicated are the numbers of the spots and correspond to the spots indicated with “nα” herein.

FIG. 31 is a photograph of an immunoblot of a two-dimensional electrophoretic pattern of proteins contained in soybean stained with serum of Soybean-allergic patient 9α. The spots indicated with a white line or enclosed in a square are spot where the serum of the soybean-allergic patient specifically reacted. The numbers (n) indicated are the numbers of the spots and correspond to the spots indicated with “nα” herein.

FIG. 4 is a diagram showing one example of results of determining peptides having the minimum number of amino acids functioning as an epitope by an overlapping technique.

FIG. 5 is a diagram showing one example of results of studying positions important for exertion of antigenicity as to the identified epitopes by an alanine scanning technique.

FIG. 6 is a graph showing results of studying cross-reactivity.

### DESCRIPTION OF EMBODIMENTS

The present invention will be described in detail below, but the present invention is not limited to them.

Unless otherwise defined herein, all scientific and technical terms used in relation to the present invention shall have meanings commonly understood by those skilled in the art.

Amino acid sequences herein are represented by one-letter amino acid symbols well known to those skilled in the art and the left end corresponds to the amino terminal and the right end corresponds to the carboxy terminal.

As referred to herein, the “allergy” refers to the state in which, when a certain antigen enters the body of a living individual sensitized to said antigen, the living individual shows a hypersensitive reaction detrimental to him/her. In blood and tissues of individuals with many food-allergic diseases, IgE antibodies specific to antigens are produced. IgE antibodies bind to mast cells or basophils. When an antigen specific to such an IgE antibody enters again the body of a patient with an allergic disease, said antigen combines with the IgE antibody bound to mast cells or basophils, and the IgE antibody crosslinks said antigen on the cell surface, resulting in physiological effects of IgE antibody-antigen interaction. Examples of such physiological effects include release of histamine, serotonin, heparin, cosinophil chemotactic factors, leucotrienes, or the like. These released substances provoke an allergic reaction resulting from the combination of an IgE antibody with particular antigens. Such allergic reactions caused by particular antigens occur through the aforementioned pathway.

In the present invention, the allergy of interest is not particularly limited as long as it is an allergy to an allergen comprising an epitope to be used. Such an allergy may include allergies to plants of the family Malvaceae, the family Rubiaceae, the family Brassicaceae, the family Poaceae, the family Cucurbitaceae, the family Compositae, the family Asparagaceae, the family Juglandaceae, the family Rhamnaceae, the family Pedaliaceae, the family Umbelliferae, the family Euphorbiaceae, the family Solanaceae, the family Bromeliaceae, the family Caricaceae, the family Rosaceae, the family Amaranthaceae, the family Vitaceae, the family Leguminosae, the family Lythraceae and/or the family Betulaceae. Examples of the plant of the family Malvaceae include nalta jute (Jew's mallow, scientific name: Corchorus olitorius) and cacao (scientific name: Theobroma cacao). Examples of the plant of the family Rubiaceae include Robusta coffee (scientific name: Coffea canephora). Examples of the plant of the family Brassicaceae include Qing geng cai (scientific name: Brassica rapa), radish (scientific name: Raphanus sativus), and rapeseed (scientific name: Brassica napus). Examples of the plant of the family Poaceae include great millet (scientific name: Sorghum bicolor), corn (scientific name: Zea mays), and bread wheat (scientific name: Triticum aestivum). Examples of the plant of the family Cucurbitaceae include melon (scientific name: Cucumis melo), bitter melon (bitter gourd, scientific name: Momordica charantia), and cucumber (scientific name: Cucumis sativus). Examples of the plant of the family Compositae include sunflower (scientific name: Helianthus annuus). Examples of the plant of the family Asparagaceae include asparagus (scientific name: Asparagus officinalis). Examples of the plant of the family Juglandaceae include Persian walnut (scientific name: Juglans regia). Examples of the plant of the family Rhamnaceae include Chinese date (scientific name: Ziziphus jujuba). Examples of the plant of the family Pedaliaceae include sesame (scientific name: Sesamum indicum). Examples of the plant of the family Umbelliferae include carrot (scientific name: Daucus carota subsp. Sativus). Examples of the plant of the family Euphorbiaceae include cassava (scientific name: Manihot esculenta). Examples of the plant of the family Solanaceae include red pepper (scientific name: Capsicum annuum) and tomato (scientific name: Solanum lycopersicum). Examples of the plant of the family Bromeliaceae include pineapple (scientific name: Ananas comosus). Examples of the plant of the family Caricaceae include papaya (scientific name: Carica papaya). Examples of the plant of the family Rosaceae include apple (scientific name: Malus domestica), wild cherry (scientific name: Prunus avium), and peach (scientific name: Prunus persica). Examples of the plant of the family Amaranthaceae include quinua (scientific name: Chenopodium quinoa), spinach (scientific name: Spinacia oleracea), and beet (scientific name: Beta vulgaris subsp. vulgaris). Examples of the plant of the family Vitaceae include common grape (scientific name: Vitis vinifera). Examples of the plant of the family Leguminosae include pigeon pea (scientific name: Cajanus cajan), adzuki bean (scientific name: Vigna angularis), wild soybean (scientific name: Glycine soja), and soybean (scientific name: Glycine max). Examples of the plant of the family Lythraceae include pomegranate (scientific name: Punica granatum). Examples of the plant of the family Betulaceae include white birch (scientific name: Betula platyphylla). The allergy can produce an allergic reaction upon contact with an antigen or consumption of the antigen. Here, the contact refers to touch to an object and, particularly, as for the human body, refers to attachment to the skin, the mucosa (eyes, lips, etc.) or the like. The consumption refers to incorporation into the body and refers to incorporation by inhalation or through an oral route. In general, allergic reactions caused by consumption of foods are particularly referred to as food allergies. In a preferred mode, the allergy may be a food allergy.

As referred to herein, the “allergy to soybean” refers to the state in which an individual has an allergic reaction caused by proteins, etc. present in soybean which act as an antigen.

As referred to herein, the “antigen” refers to a substance that provokes an allergic reaction, and is also referred to as an “allergen component”. The antigen is preferably a protein.

As referred to herein, the “protein” refers to a molecule having a structure in which naturally occurring amino acids are joined together by peptide bond. The number of amino acids present in a protein is not particularly limited. As referred to herein, the term “polypeptide” also means a molecule having a structure in which naturally occurring amino acids are joined together by peptide bond. The number of amino acids present in a polypeptide is not particularly limited. The “polypeptide” conceptually includes the “protein”. Also, polypeptides having about 2 to 50 amino acids joined together by peptide bond are in some cases called “peptides”, especially. In the case where amino acids can form different enantiomers, the amino acids are understood to form an L-enantiomer, unless otherwise indicated. The amino acid sequences of proteins, polypeptides, or peptides as used herein are represented by one-letter symbols of amino acids in accordance with standard usage and the notational convention commonly used in the art. The leftward direction represents the amino-terminal direction, and the rightward direction represents the carboxy-terminal direction. In the one-letter symbols of amino acids, X can be any substance having an amino group and a carboxyl group that can bind to amino acids at both ends, and particularly represents that any of 20 types of naturally occurring amino acids are acceptable.

### Identification of Antigens

Proteins contained in soybean were analyzed by the aforementioned technique to identify causative antigens of an allergy to soybean. To be specific, soybean proteins were subjected to two-dimensional electrophoresis under the conditions described below.

The electrophoresis in the first dimension was isoelectric focusing, which was performed using isoelectric focusing gels with a gel-strip length of 5 to 10 cm and a gel pH range of 3 to 10. The pH gradient of the gels in the direction of electrophoresis was as follows: when the total gel-strip length is taken as 1, the gel-strip length up to pH 5 is taken as “a”, the gel-strip length from pH 5 to 7 is taken as “b”, and the gel-strip length above pH 7 is taken as “c”, “a” is in the range of 0.15 to 0.3, “b” is in the range of 0.4 to 0.7, and “c” is in the range of 0.15 to 0.3. More specifically, the isoelectric focusing was performed using the IPG gels, Immobiline Drystrip (pH3-10NL), produced by GE Healthcare Bio-Sciences Corporation (hereinafter abbreviated as “GE”). The electrophoresis system used was IPGphor produced by GE. The maximum current of the electrophoresis system was limited to 75 μA per gel strip. The voltage program adopted to perform the first-dimensional isoelectric focusing was as follows: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

The electrophoresis in the second dimension was SDS-PAGE, which was performed using polyacrylamide gels whose gel concentration at the distal end in the direction of electrophoresis was set to 3 to 6% and whose gel concentration at the proximal end was set to a higher value than that at the distal end. More specifically, the SDS-PAGE was performed using NuPAGE 4-12% Bris-Tris Gels (IPG well, Mini, 1 mm) produced by Life Technologies. The electrophoresis system used was XCell SureLock Mini-Cell produced by Life Technologies. The electrophoresis was run at a constant voltage of 200 V for about 45 minutes using an electrophoresis buffer composed of 50 mM MOPS, 50 mM Tris base, 0.1% (w/v) SDS and 1 mM EDTA.

As a result, the following spots in a two-dimensional electrophoresis gel run under the conditions described above for proteins in soybean have been revealed to exhibit specific binding to IgE antibodies from soybean-allergic patients.
Spot 1α: Molecular weight 70 to 120 kDa, pl 2.5 to 6.5
Spot 2α: Molecular weight 50 to 120 kDa, pl 2.5 to 6.5
Spot 4α: Molecular weight 70 to 120 kDa, pl 3.5 to 7.5
Spot 5α: Molecular weight 70 to 170 kDa, pl 3.5 to 7.5
Spot 6α: Molecular weight 70 to 120 kDa, pl 3.5 to 7.5
Spot 7α: Molecular weight 50 to 120 kDa, pl 3.5 to 7.5
Spot 8α: Molecular weight 50 to 120 kDa, pl 3.5 to 8.5
Spot 9α: Molecular weight 50 to 120 kDa, pl 3.5 to 8.5
Spot 10α: Molecular weight 30 to 90 kDa, pl 3.5 to 7.5
Spot 11α: Molecular weight 30 to 90 kDa, pl 3.5 to 8.5
Spot 12α: Molecular weight 30 to 90 kDa, pl 3.5 to 8.5
Spot 13α: Molecular weight 30 to 90 kDa, pl 3.5 to 7.5
Spot 14α: Molecular weight 30 to 90 kDa, pl 2.5 to 6.5
Spot 15α, 16α: Molecular weight 20 to 90 kDa, pl 3.5 to 7.5
Spot 17α: Molecular weight 20 to 90 kDa, pl 3.5 to 7.5
Spot 18α: Molecular weight 30 to 90 kDa, pl 3.5 to 8.5
Spot 19α: Molecular weight 30 to 90 kDa, pl 3.5 to 8.5
Spot 25α: Molecular weight 20 to 90 kDa, pl 3.5 to 7.5
Spot 26α: Molecular weight 20 to 90 kDa, pl 3.5 to 7.5
Spot 27α: Molecular weight 20 to 90 kDa, pl 3.5 to 7.5
Spot 28α: Molecular weight 10 to 70 kDa, pl 3.5 to 7.5
Spot 29α, 30α, 31α, 32α, 33α: Molecular weight 10 to 70 kDa, pl 3.5 to 8.5
Spot 34α: Molecular weight 10 to 70 kDa, pl 5.5 to 10.5
Spot 35α: Molecular weight 20 to 90 kDa, pl 5.5 to 10.5
Spot 36α, 37α: Molecular weight 10 to 70 kDa, pl 3.5 to 8.5
Spot 38α: Molecular weight 70 to 170 kDa, pl 5.5 to 10.5
Spot 39α: Molecular weight 5 to 60 kDa, pl 5.5 to 10.5
Spot 40α, 56α: Molecular weight 5 to 60 kDa, pl 5.5 to 10.5
Spot 41α: Molecular weight 5 to 60 kDa, pl 5.5 to 10.5
Spot 42α, 43α: Molecular weight 5 to 60 kDa, pl 5.5 to 10.5
Spot 45α: Molecular weight 2 to 50 kDa, pl 3.5 to 7.5
Spot 46α: Molecular weight 2 to 50 kDa, pl 3.5 to 7.5
Spot 47α: Molecular weight 5 to 50 kDa, pl 3.5 to 7.5
Spot 48α: Molecular weight 2 to 50 kDa, pl 3.5 to 8.5
Spot 50α: Molecular weight 2 to 50 kDa, pl 2.5 to 6.5
Spot 51α: Molecular weight 5 to 60 kDa, pl 2.5 to 6.5
Spot 52α: Molecular weight 10 to 70 kDa, pl 3.5 to 7.5
Spot 53α: Molecular weight 5 to 60 kDa, pl 3.5 to 8.5
Spot 54α: Molecular weight 5 to 60 kDa, pl 3.5 to 8.5 Spot 55α: Molecular weight 30 to 90 kDa, pl 5.5 to 10.5 Antigen

### (1α) Antigen in Spot 1α

As the result of sequence identification of the antigen in spot 1α by mass spectroscopy, the amino acid sequences of SEQ ID NOs: 7-104 were detected.

Also, the mass spectroscopic data obtained for spot 1α on a mass spectrometer was analyzed by comparing the data against the NCBI protein data, and as a result, endoplasmin homolog isoform X2 (amino acid sequence: SEQ ID NO: 70, encoding nucleotide sequence: SEQ ID NO: 69) was identified as a protein comprising any of SEQ ID NOs: 71 to 87 and endoplasmin homolog isoform X2 (amino acid sequence: SEQ ID NO: 89, encoding nucleotide sequence: SEQ ID NO: 88) was identified as a protein comprising any of SEQ ID NOs: 90 to 104.

Accordingly, the antigen in spot 1α in the present invention can be any of proteins selected from the group consisting of the proteins as defined below in (1αA1-a) to (1αA1-c), (1αB1), (1αA2-a) to (1αA2-c), and (1αB2):
(1αA1-a) a protein comprising an amino acid sequence with deletion, substitution, insertion or addition of one or several amino acids in SEQ ID NO: 70;
(1αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 70;
(1αA1-c) a protein comprising an amino acid sequence encoded by a nucleotide sequence with deletion, substitution, insertion or addition of one or several nucleotides in SEQ ID NO: 69;
(1αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 69;
(1αA1-e) a protein comprising an amino acid sequence encoded by a nucleic acid that hybridizes under stringent conditions with a nucleic acid having a nucleotide sequence complementary to the nucleotide sequence of SEQ ID NO: 69;
(1αB1) a protein comprising at least one amino acid sequence selected from the group consisting of SEQ ID NOs: 71-87, preferably a protein comprising at least 2, 3, 4, 5, 6, 7, 8, 9, 10, 11, 12, 13, 14, 15, or 16 or all sequences of the amino acid sequences. As referred to above, the amino acid sequence of any of SEQ ID NOs: 71 to 87 may be an amino acid sequence derived therefrom by deletion, substitution, insertion or addition of one or several amino acids;
(1αA2-a) a protein comprising an amino acid sequence with deletion, substitution, insertion or addition of one or several amino acids in SEQ ID NO: 89;
(1αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 89;
(1αA2-c) a protein comprising an amino acid sequence encoded by a nucleotide sequence with deletion, substitution, insertion or addition of one or several nucleotides in SEQ ID NO: 88;
(1αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 88;
(1αA2-c) a protein comprising an amino acid sequence encoded by a nucleic acid that hybridizes under stringent conditions with a nucleic acid having a nucleotide sequence complementary to the nucleotide sequence of SEQ ID NO: 88;
(1αB2) a protein comprising at least one amino acid sequence selected from the group consisting of SEQ ID NOs: 90-104, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 71 to 87 may be an amino acid sequence derived therefrom by deletion, substitution, insertion or addition of one or several amino acids.

The protein selected from the group consisting of the proteins as defined above in (1αA1-a) to (1αA1-c), (1αB1), (1αA2-a) to (1αA2-c), and (1αB2) can be proteins that are found in a protein spot with a molecular weight of around 70 to 120 kDa, preferably around 80 to 110 kDa, and an isoelectric point of 2.5 to 6.5, preferably 3.0 to 6.0 on gels used in the two-dimensional electrophoresis performed under the conditions described above in the subsection titled “Identification of antigens”.

Epitopes of the aforementioned antigens (1αA1-a) to (1αA1-e) and (1αB1) reside in amino acids 546-553 (SEQ ID NO: 652), amino acids 653-661 (SEQ ID NO: 655), amino acids 781-795 (SEQ ID NO: 657), amino acids 252-266 (SEQ ID NO: 658), amino acids 771-785 (SEQ ID NO: 659), and amino acids 493-502 (SEQ ID NO: 660) of SEQ ID NO: 70. When the antigen in spot 1α contains a variation in the amino acid sequence of SEQ ID NO: 70, amino acids at positions corresponding to the region of at least one of these epitopes may retain the sequence in SEQ ID NO: 70.

From the viewpoint of improving sensitivity (a degree to which a patient can be diagnosed as being positive) or specificity (a degree to which a healthy subject is not diagnosed as being positive), the antigen in spot 1α may be the following variant.

In the epitope having the sequence of SEQ ID NO: 652, sites of amino acids other than X in XXKXEXXX or XTKXEXXX are important for binding to IgE antibodies from allergic patients. Thus, in another preferred mode, when the antigen in spot 1α contains a variation in the amino acids of SEQ ID NO: 70, amino acids 546-553 of SEQ ID NO: 70 in this antigen may have the amino acid sequence of XXKXEXXX or XTKXEXXX.

In the epitope having the sequence of SEQ ID NO: 655, sites of amino acids other than X in SEQ ID NO: 653 or 654 are important for binding to IgE antibodies from allergic patients. Thus, in another preferred mode, when the antigen in spot 1α contains a variation in the amino acid sequence of SEQ ID NO: 70, amino acids 653-661 of SEQ ID NO: 70 in this antigen may have the amino acid sequence of SEQ ID NO: 653 or 654.

In the epitope having the sequence of SEQ ID NO: 657, sites of amino acids other than X in SEQ ID NO: 656 are important for binding to IgE antibodies from allergic patients. Thus, in another preferred mode, when the antigen in spot 1α contains a variation in the amino acid sequence of SEQ ID NO: 70, amino acids 781-795 of SEQ ID NO: 70 in this antigen may have the amino acid sequence of SEQ ID NO: 656.

Preferably, a protein that is an antigen in spot 1α has activity equivalent to that of the protein having the amino acid sequence of SEQ ID NO: 70 or 89 (endoplasmin homolog isoform X2) or causative of an allergy to soybean.

### (2α) Antigen in Spot 2a

As the result of sequence identification of the antigen in spot 2α by mass spectroscopy, the amino acid sequences of SEQ ID NOs: 107-153 were detected.

Also, the mass spectroscopic data obtained for spot 2a on a mass spectrometer was analyzed by comparing the data against the NCBI protein data, and as a result, heat shock cognate protein 80 (amino acid sequence: SEQ ID NO: 106, encoding nucleotide sequence: SEQ ID NO: 105) was identified as a protein comprising any of SEQ ID NOs: 107 to 120, heat shock protein 90-1 (amino acid sequence: SEQ ID NO: 122, encoding nucleotide sequence: SEQ ID NO: 121) was identified as a protein comprising any of SEQ ID NOs: 123 to 137, and hypothetical protein GLYMA_02G302500 was identified as a protein comprising any of SEQ ID NOs: 140 to 153.

Accordingly, the antigen in spot 1α in the present invention can be any of proteins selected from the group consisting of the proteins as defined below in (2αA1-a) to (2αA1-e), (2αB1), (2αA2-a) to (2αA2-c), (2αB2), (2αA3-a) to (2αA3-c), and (2αB3):
(2αA1-a) a protein comprising an amino acid sequence with deletion, substitution, insertion or addition of one or several amino acids in SEQ ID NO: 106;
(2αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 106;
(2αA1-c) a protein comprising an amino acid sequence encoded by a nucleotide sequence with deletion, substitution, insertion or addition of one or several nucleotides in SEQ ID NO: 105;
(2αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 105;
(2αA1-c) a protein comprising an amino acid sequence encoded by a nucleic acid that hybridizes under stringent conditions with a nucleic acid having a nucleotide sequence complementary to the nucleotide sequence of SEQ ID NO: 105;
(2αB1) a protein comprising at least one amino acid sequence selected from the group consisting of SEQ ID NOs: 107-120, preferably a protein comprising at least 2, 3, 4, 5, 6, 7, 8, 9, 10, 11, 12, or 13 or all sequences of the amino acid sequences. As referred to above, the amino acid sequence of any of SEQ ID NOs: 107 to 120 may be an amino acid sequence derived therefrom by deletion, substitution, insertion or addition of one or several amino acids;
(2αA2-a) a protein comprising an amino acid sequence with deletion, substitution, insertion or addition of one or several amino acids in SEQ ID NO: 122;
(2αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 122;
(2αA2-c) a protein comprising an amino acid sequence encoded by a nucleotide sequence with deletion, substitution, insertion or addition of one or several nucleotides in SEQ ID NO: 121;
(2αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 121;
(2αA2-c) a protein comprising an amino acid sequence encoded by a nucleic acid that hybridizes under stringent conditions with a nucleic acid having a nucleotide sequence complementary to the nucleotide sequence of SEQ ID NO: 121;
(2αB2) a protein comprising at least one amino acid sequence selected from the group consisting of SEQ ID NOs: 123-137, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 123 to 137 may be an amino acid sequence derived therefrom by deletion, substitution, insertion or addition of one or several amino acids.
(2αA3-a) a protein comprising an amino acid sequence with deletion, substitution, insertion or addition of one or several amino acids in SEQ ID NO: 139;
(2αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 139;
(2αA3-c) a protein comprising an amino acid sequence encoded by a nucleotide sequence with deletion, substitution, insertion or addition of one or several nucleotides in SEQ ID NO: 138;
(2αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 138;
(2αA3-c) a protein comprising an amino acid sequence encoded by a nucleic acid that hybridizes under stringent conditions with a nucleic acid having a nucleotide sequence complementary to the nucleotide sequence of SEQ ID NO: 138;
(2αB3) a protein comprising at least one amino acid sequence selected from the group consisting of SEQ ID NOs: 140-153, preferably a protein comprising at least 2, 3, 4, 5, 6, 7, 8, 9, 10, 11, 12, or 13 or all sequences of the amino acid sequences. As referred to above, the amino acid sequence of any of SEQ ID NOs: 140 to 153 may be an amino acid sequence derived therefrom by deletion, substitution, insertion or addition of one or several amino acids.

The protein selected from the group consisting of the proteins as defined above in (2αA1-a) to (2αA1-c), (2αB1), (2αA2-a) to (2αA2-c), (2αB2), (2αA3-a) to (2αA3-c), and (2αB3) can be proteins that are found in a protein spot with a molecular weight of around 50 to 120 kDa, preferably around 60 to 110 kDa, and an isoelectric point of 2.5 to 6.5, preferably 3.0 to 6.0 on gels used in the two-dimensional electrophoresis performed under the conditions described above in the subsection titled “Identification of antigens”.

Epitopes of the aforementioned antigens (2αA1-a) to (2αA1-c) and (2αB1) reside in amino acids 42-50 (SEQ ID NO: 663), amino acids 51-65 (SEQ ID NO: 666), amino acids 69-73 (SEQ ID NO: 669), amino acids 221-235 (SEQ ID NO: 670), amino acids 351-365 (SEQ ID NO: 671), amino acids 361-375 (SEQ ID NO: 672), amino acids 411-425 (SEQ ID NO: 675), amino acids 551-565 (SEQ ID NO: 676), amino acids 671-685 (SEQ ID NO: 677), amino acids 681-695 (SEQ ID NO: 678), amino acids 41-55 (SEQ ID NO: 681), amino acids 451-465 (SEQ ID NO: 684), amino acids 271-285 (SEQ ID NO: 689), amino acids 51-59 (SEQ ID NO: 690), amino acids 65-72 (SEQ ID NO: 691), and amino acids 453-460 (SEQ ID NO: 692) of SEQ ID NO: 106. When the antigen in spot 2α contains a variation in the amino acid sequence of SEQ ID NO: 106, amino acids at positions corresponding to the region of at least one of these epitopes may retain the sequence in SEQ ID NO: 106.

From the viewpoint of improving sensitivity (a degree to which a patient can be diagnosed as being positive) or specificity (a degree to which a healthy subject is not diagnosed as being positive), the antigen in spot 2α may be the following variant.

In the epitope having the sequence of SEQ ID NO: 663, sites of amino acids other than X in DXXDKXXXX or SEQ ID NO: 662 are important for binding to IgE antibodies from allergic patients. Thus, in another preferred mode, when the antigen in spot 2α contains a variation in the amino acid sequence of SEQ ID NO: 106, amino acids 42-50 of SEQ ID NO: 106 in this antigen may have the amino acid sequence of DXXDKXXXX or SEQ ID NO: 662.

In the epitope having the sequence of SEQ ID NO: 666, sites of amino acids other than X in SEQ ID NO: 664 or 665 are important for binding to IgE antibodies from allergic patients. Thus, in another preferred mode, when the antigen in spot 2α contains a variation in the amino acid sequence of SEQ ID NO: 106, amino acids 51-65 of SEQ ID NO: 106 in this antigen may have the amino acid sequence of SEQ ID NO: 664 or 665.

In the epitope having the sequence of SEQ ID NO: 669, sites of amino acids other than X in XPDKX or SEQ ID NO: 668 are important for binding to IgE antibodies from allergic patients. Thus, in another preferred mode, when the antigen in spot 2α contains a variation in the amino acid sequence of SEQ ID NO: 106, amino acids 69-73 of SEQ ID NO: 106 in this antigen may have the amino acid sequence of XPDKX or SEQ ID NO: 668.

In the epitope having the sequence of SEQ ID NO: 675, sites of amino acids other than X in SEQ ID NO: 673 or 674 are important for binding to IgE antibodies from allergic patients. Thus, in another preferred mode, when the antigen in spot 2α contains a variation in the amino acid sequence of SEQ ID NO: 106, amino acids 411-425 of SEQ ID NO: 106 in this antigen may have the amino acid sequence of SEQ ID NO: 673 or 674.

In the epitope having the sequence of SEQ ID NO: 681, sites of amino acids other than X in XXXXDXXXXEXXXDX or SEQ ID NO: 680 are important for binding to IgE antibodies from allergic patients. Thus, in another preferred mode, when the antigen in spot 2α contains a variation in the amino acid sequence of SEQ ID NO: 106, amino acids 41-55 of SEQ ID NO: 106 in this antigen may have the amino acid sequence of XXXXDXXXXEXXXDX or SEQ ID NO: 680.

In the epitope having the sequence of SEQ ID NO: 684, sites of amino acids other than X in SEQ ID NO: 682 or 685 are important for binding to IgE antibodies from allergic patients. Thus, in another preferred mode, when the antigen in spot 2α contains a variation in the amino acid sequence of SEQ ID NO: 106, amino acids 451-465 of SEQ ID NO: 106 in this antigen may have the amino acid sequence of SEQ ID NO: 682 or 685.

In the epitope having the sequence of SEQ ID NO: 689, sites of amino acids other than X in SEQ ID NO: 688 are important for binding to IgE antibodies from allergic patients. Thus, in another preferred mode, when the antigen in spot 2α contains a variation in the amino acid sequence of SEQ ID NO: 106, amino acids 271-285 of SEQ ID NO: 106 in this antigen may have the amino acid sequence of SEQ ID NO: 688.

Preferably, a protein that is an antigen in spot 2α has activity equivalent to that of the protein having the amino acid sequence of SEQ ID NO: 106 (heat shock cognate protein 80), the protein having the amino acid sequence of SEQ ID NO: 122 (heat shock protein 90-1), or the protein having the amino acid sequence of SEQ ID NO: 139 (hypothetical protein GLYMA_02G302500), or causative of an allergy to soybean.

### (4α) Antigen in Spot 4α

As the result of sequence identification of the antigen in spot 4α by mass spectroscopy, the amino acid sequences of SEQ ID NOs: 156 and 157 were detected.

Also, the mass spectroscopic data obtained for spot 4α on a mass spectrometer was analyzed by comparing the data against the NCBI protein data, and as a result, embryo-specific urease isoform X1 (amino acid sequence: SEQ ID NO: 155, encoding nucleotide sequence: SEQ ID NO: 154) was identified as a protein comprising SEQ ID NO: 156 or 157.

Accordingly, the antigens in spot 4α in the present invention can be any protein selected from the group consisting of the proteins as defined below in (4αA-a) to (4αA-c) and (4αB):
(4αA-a) a protein comprising an amino acid sequence with deletion, substitution, insertion or addition of one or several amino acids in SEQ ID NO: 155;
(4αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 155;
(4αA-c) a protein comprising an amino acid sequence encoded by a nucleotide sequence with deletion, substitution, insertion or addition of one or several nucleotides in SEQ ID NO: 154;
(4αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 154;
(4αA-c) a protein comprising an amino acid sequence encoded by a nucleic acid that hybridizes under stringent conditions with a nucleic acid having a nucleotide sequence complementary to the nucleotide sequence of SEQ ID NO: 154;
(4αB) a protein comprising at least one amino acid sequence selected from the group consisting of SEQ ID NOs: 156 and 157, preferably a protein comprising both the amino acid sequences. As referred to above, the amino acid sequence of any of SEQ ID NOs: 156 and 157 may be an amino acid sequence derived therefrom by deletion, substitution, insertion or addition of one or several amino acids.

The proteins selected from the group consisting of (4αA-a) to (4αA-c) and (4αB) as defined above can be proteins that are found in a protein spot with a molecular weight of around 70 to 120 kDa, preferably around 80 to 110 kDa and an isoelectric point of 3.5 to 7.5, preferably 4.0 to 7.0 on gels used in the two-dimensional electrophoresis performed under the conditions described above in the subsection titled “Identification of antigens”.

Epitopes of the antigen in spot 4α reside in amino acids 121-130 (SEQ ID NO: 719), amino acids 289-295 (SEQ ID NO: 722), amino acids 293-302 (SEQ ID NO: 725), amino acids 501-515 (SEQ ID NO: 726), amino acids 571-585 (SEQ ID NO: 727), amino acids 591-605 (SEQ ID NO: 728), and amino acids 771-785 (SEQ ID NO: 731) of SEQ ID NO: 155. The antigen in spot 4α may retain the sequence in SEQ ID NO: 155 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 4α may be the following variant.

In the epitope having the sequence of SEQ ID NO: 719, sites of amino acids other than X in XXXXXXXKED or SEQ ID NO: 718 are important for binding to IgE antibodies from allergic patients. Thus, in another preferred mode, amino acids 121-130 of SEQ ID NO: 155 in the antigen in spot 4α may have the amino acid sequence of XXXXXXXKED or SEQ ID NO: 718.

In the epitope having the sequence of SEQ ID NO: 722, sites of amino acids other than X in SEQ ID NO: 720 or 721 are important for binding to IgE antibodies from allergic patients. Thus, in another preferred mode, amino acids 289-295 of SEQ ID NO: 155 in the antigen in spot 4α may have the amino acid sequence of SEQ ID NO: 720 or 721.

In the epitope having the sequence of SEQ ID NO: 725, sites of amino acids other than X in SEQ ID NO: 723 or 724 are important for binding to IgE antibodies from allergic patients. Thus, in another preferred mode, amino acids 293-302 of SEQ ID NO: 155 in the antigen in spot 4α may have the amino acid sequence of SEQ ID NO: 723 or 724.

In the epitope having the sequence of SEQ ID NO: 731, sites of amino acids other than X in SEQ ID NO: 729 or 730 are important for binding to IgE antibodies from allergic patients. Thus, in another preferred mode, amino acids 771-785 of SEQ ID NO: 155 in the antigen in spot 4α may have the amino acid sequence of SEQ ID NO: 729 or 730.

Preferably, a protein that is an antigen in spot 4α has activity equivalent to that of the protein having the amino acid sequence of SEQ ID NO: 155 (embryo-specific urease isoform X1) or causative of an allergy to soybean.

### (5α) Antigen in Spot 5α

As the result of sequence identification of the antigen in spot 5α by mass spectroscopy, the amino acid sequences of SEQ ID NOs: 160 and 161 were detected.

Also, the mass spectroscopic data obtained for spot 5α on a mass spectrometer was analyzed by comparing the data against the NCBI protein data, and as a result, alpha-1,4 glucan phosphorylase L isozyme, chloroplastic/amyloplastic (amino acid sequence: SEQ ID NO: 159, encoding nucleotide sequence: SEQ ID NO: 158) was identified as a protein comprising SEQ ID NO: 160 or 161.

Accordingly, the antigens in spot 5α in the present invention can be any protein selected from the group consisting of the proteins as defined below in (5αA-a) to (5αA-c) and (5αB):
(5αA-a) a protein comprising an amino acid sequence with deletion, substitution, insertion or addition of one or several amino acids in SEQ ID NO: 159;
(5αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 159;
(5αA-c) a protein comprising an amino acid sequence encoded by a nucleotide sequence with deletion, substitution, insertion or addition of one or several nucleotides in SEQ ID NO: 158;
(5αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 158;
(5αA-c) a protein comprising an amino acid sequence encoded by a nucleic acid that hybridizes under stringent conditions with a nucleic acid having a nucleotide sequence complementary to the nucleotide sequence of SEQ ID NO: 158;
(5αB) a protein comprising at least one amino acid sequence selected from the group consisting of SEQ ID NOs: 160 and 161, preferably a protein comprising both the amino acid sequences. As referred to above, the amino acid sequence of any of SEQ ID NOs: 160 and 161 may be an amino acid sequence derived therefrom by deletion, substitution, insertion or addition of one or several amino acids.

The proteins selected from the group consisting of (5αA-a) to (5αA-c) and (5αB) as defined above can be proteins that are found in a protein spot with a molecular weight of around 70 to 170 kDa, preferably around 80 to 160 kDa and an isoelectric point of 3.5 to 7.5, preferably 4.0 to 7.0 on gels used in the two-dimensional electrophoresis performed under the conditions described above in the subsection titled “Identification of antigens”.

Epitopes of the antigen in spot 5α reside in amino acids 861-875 (SEQ ID NO: 734), amino acids 871-885 (SEQ ID NO: 735), amino acids 891-905 (SEQ ID NO: 736), amino acids 901-915 (SEQ ID NO: 737), and amino acids 951-965 (SEQ ID NO: 738) of SEQ ID NO: 159. The antigen in spot 5α may retain the sequence in SEQ ID NO: 159 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 5α may be the following variant.

In the epitope having the sequence of SEQ ID NO: 734, sites of amino acids other than X in SEQ ID NO: 732 or 733 are important for binding to IgE antibodies from allergic patients. Thus, in another preferred mode, amino acids 861-875 of SEQ ID NO: 159 in the antigen in spot 5a may have the amino acid sequence of SEQ ID NO: 732 or 733.

Preferably, a protein that is an antigen in spot 5α has activity equivalent to that of the protein having the amino acid sequence of SEQ ID NO: 159 (alpha-1,4 glucan phosphorylase L isozyme, chloroplastic/amyloplastic) or causative of an allergy to soybean.

### (6α) Antigen in Spot 6α

As the result of sequence identification of the antigen in spot 6α by mass spectroscopy, the amino acid sequences of SEQ ID NOs: 164-172 were detected.

Also, the mass spectroscopic data obtained for spot 6α on a mass spectrometer was analyzed by comparing the data against the NCBI protein data, and as a result, aconitate hydratase 1 (amino acid sequence: SEQ ID NO: 163, encoding nucleotide sequence: SEQ ID NO: 162) was identified as a protein comprising any of SEQ ID NOs: 164 to 167 and aconitate hydratase 1 (amino acid sequence: SEQ ID NO: 169, encoding nucleotide sequence: SEQ ID NO: 168) was identified as a protein comprising any of SEQ ID NOs: 170 to 172.

Accordingly, the antigen in spot 6α in the present invention can be any of proteins selected from the group consisting of the proteins as defined below in (6αA1-a) to (6αA1-e), (6αB1), (6αA2-a) to (6αA2-c), and (6αB2):
(6αA1-a) a protein comprising an amino acid sequence with deletion, substitution, insertion or addition of one or several amino acids in SEQ ID NO: 163;
(6αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 163;
(6αA1-c) a protein comprising an amino acid sequence encoded by a nucleotide sequence with deletion, substitution, insertion or addition of one or several nucleotides in SEQ ID NO: 162;
(6αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 162;
(6αA1-e) a protein comprising an amino acid sequence encoded by a nucleic acid that hybridizes under stringent conditions with a nucleic acid having a nucleotide sequence complementary to the nucleotide sequence of SEQ ID NO: 162;
(6αB1) a protein comprising at least one amino acid sequence selected from the group consisting of SEQ ID NOs: 164-167, preferably a protein comprising at least 2, 3, or all sequences of the amino acid sequences. As referred to above, the amino acid sequence of any of SEQ ID NOs: 164 to 167 may be an amino acid sequence derived therefrom by deletion, substitution, insertion or addition of one or several amino acids;
(6αA2-a) a protein comprising an amino acid sequence with deletion, substitution, insertion or addition of one or several amino acids in SEQ ID NO: 169;
(6αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 169;
(6αA2-c) a protein comprising an amino acid sequence encoded by a nucleotide sequence with deletion, substitution, insertion or addition of one or several nucleotides in SEQ ID NO: 168;
(6αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 168;
(6αA2-c) a protein comprising an amino acid sequence encoded by a nucleic acid that hybridizes under stringent conditions with a nucleic acid having a nucleotide sequence complementary to the nucleotide sequence of SEQ ID NO: 168;
(6αB2) a protein comprising at least one amino acid sequence selected from the group consisting of SEQ ID NOs: 170-172, preferably a protein comprising at least 2, or all sequences of the amino acid sequences. As referred to above, the amino acid sequence of any of SEQ ID NOs: 170 to 172 may be an amino acid sequence derived therefrom by deletion, substitution, insertion or addition of one or several amino acids.

The protein selected from the group consisting of the proteins as defined above in (6αA1-a) to (6αA1-c), (6αB1), (6αA2-a) to (6αA2-c), and (6αB2) can be proteins that are found in a protein spot with a molecular weight of around 70 to 120 kDa, preferably around 80 to 110 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (6αA1-a) to (6αA1-e) and (6αB1) reside in amino acids 761-775 (SEQ ID NO: 739), amino acids 831-845 (SEQ ID NO: 740) of SEQ ID NO: 163. When the antigen in spot 6α contains a variation in the amino acid sequence of SEQ ID NO: 163, amino acids at positions corresponding to the region of at least one of these epitopes may retain the sequence in SEQ ID NO: 163.

Preferably, a protein that is an antigen in spot 6α has activity equivalent to that of the protein having the amino acid sequence of SEQ ID NO: 163 or 169 (aconitate hydratase 1) or causative of an allergy to soybean.

### (7α) Antigen in Spot 7α

As the result of sequence identification of the antigen in spot 7a by mass spectroscopy, the amino acid sequences of SEQ ID NOs: 175-187 were detected.

Also, the mass spectroscopic data obtained for spot 7a on a mass spectrometer was analyzed by comparing the data against the NCBI protein data, and as a result, methionine synthase (amino acid sequence: SEQ ID NO: 174, encoding nucleotide sequence: SEQ ID NO: 173) was identified as a protein comprising any of SEQ ID NOs: 175 to 187.

Accordingly, the antigens in spot 7α in the present invention can be any protein selected from the group consisting of the proteins as defined below in (7αA-a) to (7αA-c) and (7αB):
(7αA-a) a protein comprising an amino acid sequence with deletion, substitution, insertion or addition of one or several amino acids in SEQ ID NO: 174;
(7αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 174;
(7αA-c) a protein comprising an amino acid sequence encoded by a nucleotide sequence with deletion, substitution, insertion or addition of one or several nucleotides in SEQ ID NO: 173;
(7αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 173;
(7αA-c) a protein comprising an amino acid sequence encoded by a nucleic acid that hybridizes under stringent conditions with a nucleic acid having a nucleotide sequence complementary to the nucleotide sequence of SEQ ID NO: 173;
(7αB) a protein comprising at least one amino acid sequence selected from the group consisting of SEQ ID NOs: 175-187, preferably a protein comprising at least 2, 3, 4, 5, 6, 7, 8, 9, 10, 11 or 12 or all sequences of the amino acid sequences. As referred to above, the amino acid sequence of any of SEQ ID NOs: 175-187 may be an amino acid sequence derived therefrom by deletion, substitution, insertion or addition of one or several amino acids.

The proteins selected from the group consisting of (7αA-a) to (7αA-c) and (7αB) as defined above can be proteins that are found in a protein spot with a molecular weight of around 50 to 120 kDa, preferably around 60 to 110 kDa and an isoelectric point of 3.5 to 7.5, preferably 4.0 to 7.0 on gels used in the two-dimensional electrophoresis performed under the conditions described above in the subsection titled “Identification of antigens”.

Epitopes of the antigen in spot 7α reside in amino acids 471-485 (SEQ ID NO: 741), amino acids 581-595 (SEQ ID NO: 744) of SEQ ID NO: 174. The antigen in spot 7α may retain the sequence in SEQ ID NO: 174 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 7a may be the following variant.

In the epitope having the sequence of SEQ ID NO: 744, sites of amino acids other than X in SEQ ID NO: 742 or 743 are important for binding to IgE antibodies from allergic patients. Thus, in another preferred mode, amino acids 581-595 of SEQ ID NO: 174 in the antigen in spot 7a may have the amino acid sequence of SEQ ID NO: 742 or 743.

Preferably, a protein that is an antigen in spot 7α has activity equivalent to that of the protein having the amino acid sequence of SEQ ID NO: 174 (methionine synthase) or causative of an allergy to soybean.

### (8α) Antigen in Spot 8α

As the result of sequence identification of the antigen in spot 8α by mass spectroscopy, the amino acid sequences of SEQ ID NOs: 190-199 were detected.

Also, the mass spectroscopic data obtained for spot 8α on a mass spectrometer was analyzed by comparing the data against the NCBI protein data, and as a result, transketolase, chloroplastic (amino acid sequence: SEQ ID NO: 189, encoding nucleotide sequence: SEQ ID NO: 188) was identified as a protein comprising any of SEQ ID NOs: 190 to 199.

Accordingly, the antigens in spot 8α in the present invention can be any protein selected from the group consisting of the proteins as defined below in (8αA-a) to (8αA-c) and (8αB):
(8αA-a) a protein comprising an amino acid sequence with deletion, substitution, insertion or addition of one or several amino acids in SEQ ID NO: 189;
(8αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 189;
(8αA-c) a protein comprising an amino acid sequence encoded by a nucleotide sequence with deletion, substitution, insertion or addition of one or several nucleotides in SEQ ID NO: 188;
(8αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 188;
(8αA-c) a protein comprising an amino acid sequence encoded by a nucleic acid that hybridizes under stringent conditions with a nucleic acid having a nucleotide sequence complementary to the nucleotide sequence of SEQ ID NO: 188;
(8αB) a protein comprising at least one amino acid sequence selected from the group consisting of SEQ ID NOs: 190-199, preferably a protein comprising at least 2, 3, 4, 5, 6, 7, 8 or 9 or all sequences of the amino acid sequences. As referred to above, the amino acid sequence of any of SEQ ID NOs: 190-199 may be an amino acid sequence derived therefrom by deletion, substitution, insertion or addition of one or several amino acids.

The proteins selected from the group consisting of (8αA-a) to (8αA-c) and (8αB) as defined above can be proteins that are found in a protein spot with a molecular weight of around 50 to 120 kDa, preferably around 60 to 110 kDa and an isoelectric point of 3.5 to 8.5, preferably 4.0 to 8.0 on gels used in the two-dimensional electrophoresis performed under the conditions described above in the subsection titled “Identification of antigens”.

Preferably, a protein that is an antigen in spot 8α has activity equivalent to that of the protein having the amino acid sequence of SEQ ID NO: 189 (transketolase, chloroplastic) or causative of an allergy to soybean.

### (9α) Antigen in Spot 9α

As the result of sequence identification of the antigen in spot 9α by mass spectroscopy, the amino acid sequence of SEQ ID NO: 202 was detected.

Also, the mass spectroscopic data obtained for spot 9α on a mass spectrometer was analyzed by comparing the data against the NCBI protein data, and as a result, lysine-tRNA ligase, cytoplasmic-like isoform X2 (amino acid sequence: SEQ ID NO: 201, encoding nucleotide sequence: SEQ ID NO: 200) was identified as a protein comprising SEQ ID NO: 202.

Accordingly, the antigens in spot 9α in the present invention can be any protein selected from the group consisting of the proteins as defined below in (9αA-a) to (9αA-e) and (9αB):
(9αA-a) a protein comprising an amino acid sequence with deletion, substitution, insertion or addition of one or several amino acids in SEQ ID NO: 201;
(9αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 201;
(9αA-c) a protein comprising an amino acid sequence encoded by a nucleotide sequence with deletion, substitution, insertion or addition of one or several nucleotides in SEQ ID NO: 200;
(9αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 200;
(9αA-e) a protein comprising an amino acid sequence encoded by a nucleic acid that hybridizes under stringent conditions with a nucleic acid having a nucleotide sequence complementary to the nucleotide sequence of SEQ ID NO: 200;
(9αB) a protein comprising an amino acid sequence of SEQ ID NO: 202. As referred to above, the amino acid sequence(s) of SEQ ID NO: 202 may be an amino acid sequence(s) derived therefrom by deletion, substitution, insertion or addition of one or several amino acids.

The proteins selected from the group consisting of (9αA-a) to (9αA-c) and (9αB) as defined above can be proteins that are found in a protein spot with a molecular weight of around 50 to 120 kDa, preferably around 60 to 110 kDa and an isoelectric point of 3.5 to 8.5, preferably 4.0 to 8.0 on gels used in the two-dimensional electrophoresis performed under the conditions described above in the subsection titled “Identification of antigens”.

Epitopes of the antigen in spot 9α reside in amino acids 236-245 (SEQ ID NO: 747), amino acids 283-290 (SEQ ID NO: 749) of SEQ ID NO: 201. The antigen in spot 9α may retain the sequence in SEQ ID NO: 201 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 9α may be the following variant.

In the epitope having the sequence of SEQ ID NO: 747, sites of amino acids other than X in XXEXXXXKDX or SEQ ID NO: 746 are important for binding to IgE antibodies from allergic patients. Thus, in another preferred mode, amino acids 236-245 of SEQ ID NO: 201 in the antigen in spot 9α may have the amino acid sequence of XXEXXXXKDX or SEQ ID NO: 746.

In the epitope having the sequence of SEQ ID NO: 749, sites of amino acids other than X in SEQ ID NO: 748 are important for binding to IgE antibodies from allergic patients. Thus, in another preferred mode, amino acids 283-290 of SEQ ID NO: 201 in the antigen in spot 9α may have the amino acid sequence of SEQ ID NO: 748.

Preferably, a protein that is an antigen in spot 9α has activity equivalent to that of the protein having the amino acid sequence of SEQ ID NO: 201 (lysine-tRNA ligase, cytoplasmic-like isoform X2) or causative of an allergy to soybean.

### (10α) Antigen in Spot 10α

As the result of sequence identification of the antigen in spot 10α by mass spectroscopy, the amino acid sequences of SEQ ID NOs: 205-231 were detected.

Also, the mass spectroscopic data obtained for spot 10α on a mass spectrometer was analyzed by comparing the data against the NCBI protein data, and as a result, chaperonin CPN60-2, mitochondrial (amino acid sequence: SEQ ID NO: 204, encoding nucleotide sequence: SEQ ID NO: 203) was identified as a protein comprising any of SEQ ID NOs: 205 to 218 and chaperonin CPN60-2, mitochondrial-like isoform X1 (amino acid sequence: SEQ ID NO: 220, encoding nucleotide sequence: SEQ ID NO: 219) was identified as a protein comprising any of SEQ ID NOs: 221 to 231.

Accordingly, the antigen in spot 10α in the present invention can be any of proteins selected from the group consisting of the proteins as defined below in (10αA1-a) to (10αA1-c), (10αB1), (10αA2-a) to (10αA2-c), and (10αB2):
(10αA1-a) a protein comprising an amino acid sequence with deletion, substitution, insertion or addition of one or several amino acids in SEQ ID NO: 204;
(10αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 204;
(10αA1-c) a protein comprising an amino acid sequence encoded by a nucleotide sequence with deletion, substitution, insertion or addition of one or several nucleotides in SEQ ID NO: 203;
(10αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 203;
(10αA1-c) a protein comprising an amino acid sequence encoded by a nucleic acid that hybridizes under stringent conditions with a nucleic acid having a nucleotide sequence complementary to the nucleotide sequence of SEQ ID NO: 203;
(10αB1) a protein comprising at least one amino acid sequence selected from the group consisting of SEQ ID NOs: 205-218, preferably a protein comprising at least 2, 3, 4, 5, 6, 7, 8, 9, 10, 11, 12, or 13 or all sequences of the amino acid sequences. As referred to above, the amino acid sequence of any of SEQ ID NOs: 205 to 218 may be an amino acid sequence derived therefrom by deletion, substitution, insertion or addition of one or several amino acids;
(10αA2-a) a protein comprising an amino acid sequence with deletion, substitution, insertion or addition of one or several amino acids in SEQ ID NO: 220;
(10αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 220;
(10αA2-c) a protein comprising an amino acid sequence encoded by a nucleotide sequence with deletion, substitution, insertion or addition of one or several nucleotides in SEQ ID NO: 219; (10A2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 219;
(10αA2-e) a protein comprising an amino acid sequence encoded by a nucleic acid that hybridizes under stringent conditions with a nucleic acid having a nucleotide sequence complementary to the nucleotide sequence of SEQ ID NO: 219;
(10β2) a protein comprising at least one amino acid sequence selected from the group consisting of SEQ ID NOs: 221-231, preferably a protein comprising at least 2, 3, 4, 5, 6, 7, 8, 9, or 10, or all sequences of the amino acid sequences. As referred to above, the amino acid sequence of any of SEQ ID NOs: 221 to 231 may be an amino acid sequence derived therefrom by deletion, substitution, insertion or addition of one or several amino acids.

The protein selected from the group consisting of the proteins as defined above in (10αA1-a) to (10αA1-c), (10αB1), (10αA2-a) to (10αA2-e), and (10αB2) can be proteins that are found in a protein spot with a molecular weight of around 30 to 90 kDa, preferably around 40 to 80 kDa, and an isoelectric point of 3.5 to 7.5, preferably 4.0 to 7.0 on gels used in the two-dimensional electrophoresis performed under the conditions described above in the subsection titled “Identification of antigens”.

Preferably, a protein that is an antigen in spot 10α has activity equivalent to that of the protein having the amino acid sequence of SEQ ID NO: 204 (haperonin CPN60-2, mitochondrial) or the protein having the amino acid sequence of SEQ ID NO: 220 (chaperonin CPN60-2, mitochondrial-like isoform X1) or causative of an allergy to soybean.

### (11α) Antigen in Spot 11α

As the result of sequence identification of the antigen in spot 11a by mass spectroscopy, the amino acid sequences of SEQ ID NOs: 234-237 were detected.

Also, the mass spectroscopic data obtained for spot 11a on a mass spectrometer was analyzed by comparing the data against the NCBI protein data, and as a result, polyadenylate-binding protein 8 isoform X3 (amino acid sequence: SEQ ID NO: 233, encoding nucleotide sequence: SEQ ID NO: 232) was identified as a protein comprising any of SEQ ID NOs: 234 to 237.

Accordingly, the antigens in spot 11α in the present invention can be any protein selected from the group consisting of the proteins as defined below in (11αA-a) to (11αA-c) and (11αB):
(11αA-a) a protein comprising an amino acid sequence with deletion, substitution, insertion or addition of one or several amino acids in SEQ ID NO: 233;
(11αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 233;
(11αA-c) a protein comprising an amino acid sequence encoded by a nucleotide sequence with deletion, substitution, insertion or addition of one or several nucleotides in SEQ ID NO: 232;
(11αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 232;
(11αA-c) a protein comprising an amino acid sequence encoded by a nucleic acid that hybridizes under stringent conditions with a nucleic acid having a nucleotide sequence complementary to the nucleotide sequence of SEQ ID NO: 232;
(11αB) a protein comprising at least one amino acid sequence selected from the group consisting of SEQ ID NOs: 234-237, preferably a protein comprising at least 2 or 3 or all sequences of the amino acid sequences. As referred to above, the amino acid sequence of any of SEQ ID NOs: 234-237 may be an amino acid sequence derived therefrom by deletion, substitution, insertion or addition of one or several amino acids.

The proteins selected from the group consisting of (11αA-a) to (11αA-c) and (11αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

Epitopes of the antigen in spot 11a reside in amino acids 454-463 (SEQ ID NO: 753), amino acids 363-370 (SEQ ID NO: 754) of SEQ ID NO: 233. The antigen in spot 11α may retain the sequence in SEQ ID NO: 233 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 11a may be the following variant.

In the epitope having the sequence of SEQ ID NO: 753, sites of amino acids other than X in SEQ ID NO: 751 or 752 are important for binding to IgE antibodies from allergic patients. Thus, in another preferred mode, amino acids 454-463 of SEQ ID NO: 223 in the antigen in spot 11α may have the amino acid sequence of SEQ ID NO: 751 or 752.

Preferably, a protein that is an antigen in spot 11α has activity equivalent to that of the protein having the amino acid sequence of SEQ ID NO: 233 (polyadenylate-binding protein 8 isoform X3) or causative of an allergy to soybean.

### (12α) Antigen in Spot 12α

As the result of sequence identification of the antigen in spot 12α by mass spectroscopy, the amino acid sequence of SEQ ID NO: 240 was detected.

Also, the mass spectroscopic data obtained for spot 12α on a mass spectrometer was analyzed by comparing the data against the NCBI protein data, and as a result, pyrophosphate-fructose 6-phosphate 1-phosphotransferase subunit alpha-like (amino acid sequence: SEQ ID NO: 239, encoding nucleotide sequence: SEQ ID NO: 238) was identified as a protein comprising SEQ ID NO: 240.

Accordingly, the antigens in spot 12α in the present invention can be any protein selected from the group consisting of the proteins as defined below in (12αA-a) to (12αA-c) and (12αB):
(12αA-a) a protein comprising an amino acid sequence with deletion, substitution, insertion or addition of one or several amino acids in SEQ ID NO: 239;
(12αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 239;
(12αA-c) a protein comprising an amino acid sequence encoded by a nucleotide sequence with deletion, substitution, insertion or addition of one or several nucleotides in SEQ ID NO: 238;
(12αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 238;
(12αA-c) a protein comprising an amino acid sequence encoded by a nucleic acid that hybridizes under stringent conditions with a nucleic acid having a nucleotide sequence complementary to the nucleotide sequence of SEQ ID NO: 238;
(12αB) a protein comprising an amino acid sequence of SEQ ID NO: 240. As referred to above, the amino acid sequence(s) of SEQ ID NO: 240 may be an amino acid sequence(s) derived therefrom by deletion, substitution, insertion or addition of one or several amino acids.

The proteins selected from the group consisting of (12αA-a) to (12αA-c) and (12αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

An epitope of the antigen in spot 12α resides in amino acids 551-565 (SEQ ID NO: 755) of SEQ ID NO: 239. The antigen in spot 11α may retain the sequence in SEQ ID NO: 239 as to amino acids at positions corresponding to the region of this epitope.

Preferably, a protein that is an antigen in spot 12α has activity equivalent to that of the protein having the amino acid sequence of SEQ ID NO: 239 (pyrophosphate-fructose 6-phosphate 1-phosphotransferase subunit alpha-like) or causative of an allergy to soybean.

### (13α) Antigen in Spot 13α

As the result of sequence identification of the antigen in spot 13α by mass spectroscopy, the amino acid sequences of SEQ ID NOs: 243-278 were detected.

Also, the mass spectroscopic data obtained for spot 13α on a mass spectrometer was analyzed by comparing the data against the NCBI protein data, and as a result, protein disulfide isomerase-like protein precursor (amino acid sequence: SEQ ID NO: 242, encoding nucleotide sequence: SEQ ID NO: 241) was identified as a protein comprising any of SEQ ID NOs: 243 to 252 and protein disulfide-isomerase (amino acid sequence: SEQ ID NO: 254, encoding nucleotide sequence: SEQ ID NO: 253) was identified as a protein comprising any of SEQ ID NOs: 255 to 278.

Accordingly, the antigen in spot 13α in the present invention can be any of proteins selected from the group consisting of the proteins as defined below in (13αA1-a) to (13αA1-e), (13αB1), (13αA2-a) to (13αA2-c), and (13αB2):
(13αA1-a) a protein comprising an amino acid sequence with deletion, substitution, insertion or addition of one or several amino acids in SEQ ID NO: 242;
(13αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 242;
(13αA1-c) a protein comprising an amino acid sequence encoded by a nucleotide sequence with deletion, substitution, insertion or addition of one or several nucleotides in SEQ ID NO: 241;
(13A1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 241;
(13αA1-c) a protein comprising an amino acid sequence encoded by a nucleic acid that hybridizes under stringent conditions with a nucleic acid having a nucleotide sequence complementary to the nucleotide sequence of SEQ ID NO: 241;
(13αB1) a protein comprising at least one amino acid sequence selected from the group consisting of SEQ ID NOs: 243-252, preferably a protein comprising at least 2, 3, 4, 5, 6, 7, 8, or 9, or all sequences of the amino acid sequences. As referred to above, the amino acid sequence of any of SEQ ID NOs: 205 to 218 may be an amino acid sequence derived therefrom by deletion, substitution, insertion or addition of one or several amino acids;
(13αA2-a) a protein comprising an amino acid sequence with deletion, substitution, insertion or addition of one or several amino acids in SEQ ID NO: 254;
(13αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 254;
(13αA2-c) a protein comprising an amino acid sequence encoded by a nucleotide sequence with deletion, substitution, insertion or addition of one or several nucleotides in SEQ ID NO: 253;
(13αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 253;
(13αA2-e) a protein comprising an amino acid sequence encoded by a nucleic acid that hybridizes under stringent conditions with a nucleic acid having a nucleotide sequence complementary to the nucleotide sequence of SEQ ID NO: 253;
(13αB2) a protein comprising at least one amino acid sequence selected from the group consisting of SEQ ID NOs: 255-278, preferably a protein comprising at least 2, 3, 4, 5, 6, 7, 8, 9, 10, 11, 12, 13, 14, 15, 16, 17, 18, 19, 20, 21, 22, or 23, or all sequences of the amino acid sequences. As referred to above, the amino acid sequence of any of SEQ ID NOs: 221 to 231 may be an amino acid sequence derived therefrom by deletion, substitution, insertion or addition of one or several amino acids.

The protein selected from the group consisting of the proteins as defined above in (13αA1-a) to (13αA1-c), (13αB1), (13αA2-a) to (13αA2-c), and (13αB2) can be proteins that are found in a protein spot with a molecular weight of around 30 to 90 kDa, preferably around 40 to 80 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (13αA1-a) to (13αA1-c) and (13αB1) reside in amino acids 471-485 (SEQ ID NO: 758) of SEQ ID NO: 242. When the antigen in spot 13α contains a variation in the amino acid sequence of SEQ ID NO: 242, this antigen may retain the sequence in SEQ ID NO: 242 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 13α may be the following variant.

In the epitope having the sequence of SEQ ID NO: 758, sites of amino acids other than X in XXGXXXXXXXXXXXE or SEQ ID NO: 757 are important for binding to IgE antibodies from allergic patients. Thus, in another preferred mode, when the antigen in spot 13α contains a variation in the amino acid sequence of SEQ ID NO: 242, amino acids 471-485 of SEQ ID NO: 242 in this antigen may have the amino acid sequence of XXGXXXXXXXXXXXE or SEQ ID NO: 757.

Preferably, a protein that is an antigen in spot 13α has activity equivalent to that of the protein having the amino acid sequence of SEQ ID NO: 242 (protein disulfide isomerase-like protein precursor) or the protein having the amino acid sequence of SEQ ID NO: 254 (protein disulfide-isomerase) or causative of an allergy to soybean.

### (14α) Antigen in Spot 14α

As the result of sequence identification of the antigen in spot 14α by mass spectroscopy, the amino acid sequences of SEQ ID NOs: 281-289 were detected.

Also, the mass spectroscopic data obtained for spot 14α on a mass spectrometer was analyzed by comparing the data against the NCBI protein data, and as a result, V-type proton ATPase subunit B2 (amino acid sequence: SEQ ID NO: 280, encoding nucleotide sequence: SEQ ID NO: 279) was identified as a protein comprising any of SEQ ID NOs: 281 to 284 and V-type proton ATPase subunit B2 (amino acid sequence: SEQ ID NO: 286, encoding nucleotide sequence: SEQ ID NO: 285) was identified as a protein comprising any of SEQ ID NOs: 287 to 289.

Accordingly, the antigen in spot 14α in the present invention can be any of proteins selected from the group consisting of the proteins as defined below in (14αA1-a) to (14αA1-c), (14αB1), (14αA2-a) to (14αA2-c), and (14«B2):
(14αA1-a) a protein comprising an amino acid sequence with deletion, substitution, insertion or addition of one or several amino acids in SEQ ID NO: 280;
(14αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 280;
(14αA1-c) a protein comprising an amino acid sequence encoded by a nucleotide sequence with deletion, substitution, insertion or addition of one or several nucleotides in SEQ ID NO: 279;
(14αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 279;
(14αA1-c) a protein comprising an amino acid sequence encoded by a nucleic acid that hybridizes under stringent conditions with a nucleic acid having a nucleotide sequence complementary to the nucleotide sequence of SEQ ID NO: 279;
(14αB1) a protein comprising at least one amino acid sequence selected from the group consisting of SEQ ID NOs: 281-284, preferably a protein comprising at least 2, 3, or all sequences of the amino acid sequences. As referred to above, the amino acid sequence of any of SEQ ID NOs: 281 to 284 may be an amino acid sequence derived therefrom by deletion, substitution, insertion or addition of one or several amino acids;
(14αA2-a) a protein comprising an amino acid sequence with deletion, substitution, insertion or addition of one or several amino acids in SEQ ID NO: 286;
(14αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 286;
(14αA2-c) a protein comprising an amino acid sequence encoded by a nucleotide sequence with deletion, substitution, insertion or addition of one or several nucleotides in SEQ ID NO: 285;
(14αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 285;
(14αA2-e) a protein comprising an amino acid sequence encoded by a nucleic acid that hybridizes under stringent conditions with a nucleic acid having a nucleotide sequence complementary to the nucleotide sequence of SEQ ID NO: 285;
(14«B2) a protein comprising at least one amino acid sequence selected from the group consisting of SEQ ID NOs: 287-289, preferably a protein comprising at least 2, or all sequences of the amino acid sequences. As referred to above, the amino acid sequence of any of SEQ ID NOs: 287 to 289 may be an amino acid sequence derived therefrom by deletion, substitution, insertion or addition of one or several amino acids.

The protein selected from the group consisting of the proteins as defined above in (14αA1-a) to (14αA1-c), (14αB1), (14αA2-a) to (14αA2-c), and (14αB2) can be proteins that are found in a protein spot with a molecular weight of around 30 to 90 kDa, preferably around 40 to 80 kDa, and an isoelectric point of 2.5 to 6.5, preferably 3.0 to 6.0 on gels used in the two-dimensional electrophoresis performed under the conditions described above in the subsection titled “Identification of antigens”.

Epitopes of the aforementioned antigens (14αA1-a) to (14αA1-c) and (14αB1) reside in amino acids 191-205 (SEQ ID NO: 761) and amino acids 391-405 (SEQ ID NO: 764) of SEQ ID NO: 280. When the antigen in spot 14α contains a variation in the amino acid sequence of SEQ ID NO: 280, this antigen may retain the sequence in SEQ ID NO: 280 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 14α may be the following variant.

In the epitope having the sequence of SEQ ID NO: 761, sites of amino acids other than X in SEQ ID NO: 759 or 760 are important for binding to IgE antibodies from allergic patients. Thus, in another preferred mode, when the antigen in spot 14α contains a variation in the amino acid sequence of SEQ ID NO: 280, amino acids 191-205 of SEQ ID NO: 280 in this antigen may each independently have the amino acid sequence of SEQ ID NO: 759 or 760.

In the epitope having the sequence of SEQ ID NO: 764, sites of amino acids other than X in SEQ ID NO: 762 or 763 are important for binding to IgE antibodies from allergic patients. Thus, in another preferred mode, when the antigen in spot 14α contains a variation in the amino acid sequence of SEQ ID NO: 280, amino acids 391-405 of SEQ ID NO: 280 in this antigen may each independently have the amino acid sequence of SEQ ID NO: 762 or 763.

Preferably, a protein that is an antigen in spot 14α has activity equivalent to that of the protein having the amino acid sequence of SEQ ID NO: 280 or 286 (V-type proton ATPase subunit B2) or causative of an allergy to soybean.

### (15α, 16α) Antigens in Spots 15α, 16α

As the result of sequence identification of the antigens in spots 15α and 16α by mass spectroscopy, the amino acid sequences of SEQ ID NOs: 292-318 were detected.

Also, the mass spectroscopic data obtained for spots 15α, 16a on a mass spectrometer was analyzed by comparing the data against the NCBI protein data, and as a result, UTP-glucose-1-phosphate uridylyltransferase (amino acid sequence: SEQ ID NO: 291, encoding nucleotide sequence: SEQ ID NO: 290) was identified as a protein comprising any of SEQ ID NOs: 292 to 304 and the hypothetical protein GLYMA_02G241100 (amino acid sequence: SEQ ID NO: 306, encoding nucleotide sequence: SEQ ID NO: 305) was identified as a protein comprising any of SEQ ID NOs: 307 to 318.

Accordingly, the antigen in spot 15α, 16α in the present invention can be any of proteins selected from the group consisting of the proteins as defined below in (15, 16αA1-a) to (15, 16αA1-c), (15, 16αB1), (15, 16αA2-a) to (15, 16αA2-c), and (15, 16αB2): (15, 16αA1-a) a protein comprising an amino acid sequence with deletion, substitution, insertion or addition of one or several amino acids in SEQ ID NO: 291; (15, 16αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 291;
(15, 16αA1-c) a protein comprising an amino acid sequence encoded by a nucleotide sequence with deletion, substitution, insertion or addition of one or several nucleotides in SEQ ID NO: 290;
(15, 16αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 290;
(15, 16αA1-e) a protein comprising an amino acid sequence encoded by a nucleic acid that hybridizes under stringent conditions with a nucleic acid having a nucleotide sequence complementary to the nucleotide sequence of SEQ ID NO: 290;
(15, 16αB1) a protein comprising at least one amino acid sequence selected from the group consisting of SEQ ID NOs: 292-304, preferably a protein comprising at least 2, 3, 4, 5, 6, 7, 8, 9, 10, 11, or 12 or all sequences of the amino acid sequences. As referred to above, the amino acid sequence of any of SEQ ID NOs: 292 to 304 may be an amino acid sequence derived therefrom by deletion, substitution, insertion or addition of one or several amino acids;
(15, 16αA2-a) a protein comprising an amino acid sequence with deletion, substitution, insertion or addition of one or several amino acids in SEQ ID NO: 306;
(15, 16αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 306;
(15, 16αA2-c) a protein comprising an amino acid sequence encoded by a nucleotide sequence with deletion, substitution, insertion or addition of one or several nucleotides in SEQ ID NO: 305;
(15, 16αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 305;
(15, 16αA2-e) a protein comprising an amino acid sequence encoded by a nucleic acid that hybridizes under stringent conditions with a nucleic acid having a nucleotide sequence complementary to the nucleotide sequence of SEQ ID NO: 305;
(15, 16αB2) a protein comprising at least one amino acid sequence selected from the group consisting of SEQ ID NOs: 307-318, preferably a protein comprising at least 2, 3, 4, 5, 6, 7, 8, 9, 10, or 11 or all sequences of the amino acid sequences. As referred to above, the amino acid sequence of any of SEQ ID NOs: 307 to 318 may be an amino acid sequence derived therefrom by deletion, substitution, insertion or addition of one or several amino acids.

The protein selected from the group consisting of the proteins as defined above in (15, 16αA1-a) to (15, 16αA1-c), (15, 16αB1), (15, 16αA2-a) to (15, 16αA2-c), and (15, 16αB2) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (15, 16αA1-a) to (15, 16αA1-e) and (15, 16β1) reside in amino acids 211-225 (SEQ ID NO: 766) of SEQ ID NO: 291. When the antigen in spot 15α, 16α contains a variation in the amino acid sequence of SEQ ID NO: 291, this antigen may retain the sequence in SEQ ID NO: 291 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 15α, 16a may be the following variant.

In the epitope having the sequence of SEQ ID NO: 766, sites of amino acids other than X in XGKXXXXXXXXXXXX are important for binding to IgE antibodies from allergic patients. Thus, in another preferred mode, when the antigen in spot 15α, 16α contains a variation in the amino acid sequence of SEQ ID NO: 291, amino acids 211-225 of SEQ ID NO: 291 in this antigen may have the amino acid sequence of XGKXXXXXXXXXXXX.

Preferably, a protein that is an antigen in spot 15α or 16α has activity equivalent to that of the protein having the amino acid sequence of SEQ ID NO: 291 (UTP-glucose-1-phosphate uridylyltransferase) or the protein having the amino acid sequence of SEQ ID NO: 306 (hypothetical protein GLYMA_02G241100) or causative of an allergy to soybean.

### (17α) Antigen in Spot 17α

As the result of sequence identification of the antigen in spot 17 by mass spectroscopy, the amino acid sequences of SEQ ID NOs: 321-332 were detected.

Also, the mass spectroscopic data obtained for spot 17a on a mass spectrometer was analyzed by comparing the data against the NCBI protein data, and as a result, guanosine nucleotide diphosphate dissociation inhibitor 2 (amino acid sequence: SEQ ID NO: 320, encoding nucleotide sequence: SEQ ID NO: 319) was identified as a protein comprising any of SEQ ID NOs: 321 to 325 and rab GDP dissociation inhibitor alpha-like (amino acid sequence: SEQ ID NO: 327, encoding nucleotide sequence: SEQ ID NO: 326) was identified as a protein comprising any of SEQ ID NOs: 328 to 332.

Accordingly, the antigen in spot 17α in the present invention can be any of proteins selected from the group consisting of the proteins as defined below in (17αA1-a) to (17αA1-c), (17αB1), (17αA2-a) to (17αA2-c), and (17αB2):
(17αA1-a) a protein comprising an amino acid sequence with deletion, substitution, insertion or addition of one or several amino acids in SEQ ID NO: 320;
(17αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 320;
(17αA1-c) a protein comprising an amino acid sequence encoded by a nucleotide sequence with deletion, substitution, insertion or addition of one or several nucleotides in SEQ ID NO: 319;
(17αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 319;
(17αA1-c) a protein comprising an amino acid sequence encoded by a nucleic acid that hybridizes under stringent conditions with a nucleic acid having a nucleotide sequence complementary to the nucleotide sequence of SEQ ID NO: 319;
(17αB1) a protein comprising at least one amino acid sequence selected from the group consisting of SEQ ID NOs: 321-325, preferably a protein comprising at least 2, 3, or 4 or all sequences of the amino acid sequences. As referred to above, the amino acid sequence of any of SEQ ID NOs: 321 to 325 may be an amino acid sequence derived therefrom by deletion, substitution, insertion or addition of one or several amino acids.
(17αA2-a) a protein comprising an amino acid sequence with deletion, substitution, insertion or addition of one or several amino acids in SEQ ID NO: 327;
(17αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 327;
(17αA2-c) a protein comprising an amino acid sequence encoded by a nucleotide sequence with deletion, substitution, insertion or addition of one or several nucleotides in SEQ ID NO: 326;
(17αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 326;
(17αA2-c) a protein comprising an amino acid sequence encoded by a nucleic acid that hybridizes under stringent conditions with a nucleic acid having a nucleotide sequence complementary to the nucleotide sequence of SEQ ID NO: 326;
(17αB2) a protein comprising at least one amino acid sequence selected from the group consisting of SEQ ID NOs: 328-332, preferably a protein comprising at least 2, 3, or 4 or all sequences of the amino acid sequences. As referred to above, the amino acid sequence of any of SEQ ID NOs: 328 to 332 may be an amino acid sequence derived therefrom by deletion, substitution, insertion or addition of one or several amino acids;

The protein selected from the group consisting of the proteins as defined above in (17αA1-a) to (17αA1-c), (17αB1), (17αA2-a) to (17αA2-c), and (17αB2) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (17αA1-a) to (17αA1-c) and (17αB1) reside in amino acids 171-185 (SEQ ID NO: 767) of SEQ ID NO: 320. When the antigen in spot 17α contains a variation in the amino acid sequence of SEQ ID NO: 320, this antigen may retain the sequence in SEQ ID NO: 320 as to amino acids at positions corresponding to the region of at least one of these epitopes.

Preferably, a protein that is an antigen in spot 17a has activity equivalent to that of the protein having the amino acid sequence of SEQ ID NO: 320 (guanosine nucleotide diphosphate dissociation inhibitor 2) or the protein having the amino acid sequence of SEQ ID NO: 327 (rab GDP dissociation inhibitor alpha-like) or causative of an allergy to soybean.

### (18α) Antigen in Spot 18α

As the result of sequence identification of the antigen in spot 18α by mass spectroscopy, the amino acid sequences of SEQ ID NOs: 335-339 were detected.

Also, the mass spectroscopic data obtained for spot 18α on a mass spectrometer was analyzed by comparing the data against the NCBI protein data, and as a result, hypothetical protein GLYMA_10G028300 (amino acid sequence: SEQ ID NO: 334, encoding nucleotide sequence: SEQ ID NO: 333) was identified as a protein comprising any of SEQ ID NOs: 335 to 339.

Accordingly, the antigens in spot 18α in the present invention can be any protein selected from the group consisting of the proteins as defined below in (18αA-a) to (18αA-c) and (18αB):
(18αA-a) a protein comprising an amino acid sequence with deletion, substitution, insertion or addition of one or several amino acids in SEQ ID NO: 334;
(18αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 334;
(18αA-c) a protein comprising an amino acid sequence encoded by a nucleotide sequence with deletion, substitution, insertion or addition of one or several nucleotides in SEQ ID NO: 333;
(18αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 333;
(18αA-c) a protein comprising an amino acid sequence encoded by a nucleic acid that hybridizes under stringent conditions with a nucleic acid having a nucleotide sequence complementary to the nucleotide sequence of SEQ ID NO: 333;
(18αB) a protein comprising at least one amino acid sequence selected from the group consisting of SEQ ID NOs: 335-339, preferably a protein comprising at least 2, 3 or 4 or all sequences of the amino acid sequences. As referred to above, the amino acid sequence of any of SEQ ID NOs: 335-339 may be an amino acid sequence derived therefrom by deletion, substitution, insertion or addition of one or several amino acids.

The proteins selected from the group consisting of (18αA-a) to (18αA-e) and (18αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

Epitopes of the antigen in spot 18α reside in amino acids 52-60 (SEQ ID NO: 750), amino acids 81-95 (SEQ ID NO: 768), amino acids 101-115 (SEQ ID NO: 771), amino acids 281-295 (SEQ ID NO: 772), amino acids 491-500 (SEQ ID NO: 775) of SEQ ID NO: 334. The antigen in spot 18α may retain the sequence in SEQ ID NO: 334 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 18α may be the following variant.

In the epitope having the sequence of SEQ ID NO: 771, sites of amino acids other than X in SEQ ID NO: 769 or 770 are important for binding to IgE antibodies from allergic patients. Thus, in another preferred mode, amino acids 101-115 of SEQ ID NO: 334 in the antigen in spot 18α may have the amino acid sequence of SEQ ID NO: 769 or 770.

In the epitope having the sequence of SEQ ID NO: 775, sites of amino acids other than X in SEQ ID NO: 773 or 774 are important for binding to IgE antibodies from allergic patients. Thus, in another preferred mode, amino acids 491-500 of SEQ ID NO: 334 in the antigen in spot 18α may have the amino acid sequence of SEQ ID NO: 773 or 774.

Preferably, a protein that is an antigen in spot 18α has activity equivalent to that of the protein having the amino acid sequence of SEQ ID NO: 334 (hypothetical protein GLYMA_10G028300) or causative of an allergy to soybean.

### (19α) Antigen in Spot 19α

As the result of sequence identification of the antigen in spot 19α by mass spectroscopy, the amino acid sequences of SEQ ID NOs: 342-346 were detected.

Also, the mass spectroscopic data obtained for spot 19α on a mass spectrometer was analyzed by comparing the data against the NCBI protein data, and as a result, sucrose binding protein homolog S-64 (amino acid sequence: SEQ ID NO: 341, encoding nucleotide sequence: SEQ ID NO: 340) was identified as a protein comprising any of SEQ ID NOs: 342 to 346.

Accordingly, the antigens in spot 19α in the present invention can be any protein selected from the group consisting of the proteins as defined below in (19αA-a) to (19αA-c) and (19αB):
(19αA-a) a protein comprising an amino acid sequence with deletion, substitution, insertion or addition of one or several amino acids in SEQ ID NO: 341;
(19αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 341;
(19αA-c) a protein comprising an amino acid sequence encoded by a nucleotide sequence with deletion, substitution, insertion or addition of one or several nucleotides in SEQ ID NO: 340;
(19αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 340;
(19αA-c) a protein comprising an amino acid sequence encoded by a nucleic acid that hybridizes under stringent conditions with a nucleic acid having a nucleotide sequence complementary to the nucleotide sequence of SEQ ID NO: 340;
(19αB) a protein comprising at least one amino acid sequence selected from the group consisting of SEQ ID NOs: 342-346, preferably a protein comprising at least 2, 3 or 4 or all sequences of the amino acid sequences. As referred to above, the amino acid sequence of any of SEQ ID NOs: 342-346 may be an amino acid sequence derived therefrom by deletion, substitution, insertion or addition of one or several amino acids.

The proteins selected from the group consisting of (19αA-a) to (19αA-c) and (19αB) as defined above can be proteins that are found in a protein spot with a molecular weight of around 30 to 90 kDa, preferably around 40 to 80 kDa and an isoelectric point of 3.5 to 8.5, preferably 4.0 to 8.0 on gels used in the two-dimensional electrophoresis performed under the conditions described above in the subsection titled “Identification of antigens”.

Epitopes of the antigen in spot 19α reside in amino acids 191-205 (SEQ ID NO: 778), amino acids 475-489 (SEQ ID NO: 781), amino acids 81-95 (SEQ ID NO: 784), amino acids 436-450 (SEQ ID NO: 786), amino acids 21-30 (SEQ ID NO: 787) of SEQ ID NO: 341. The antigen in spot 19α may retain the sequence in SEQ ID NO: 341 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 19α may be the following variant.

In the epitope having the sequence of SEQ ID NO: 778, sites of amino acids other than X in SEQ ID NO: 776 or 778 are important for binding to IgE antibodies from allergic patients. Thus, in another preferred mode, amino acids 191-205 of SEQ ID NO: 341 in the antigen in spot 19α may have the amino acid sequence of SEQ ID NO: 776 or 778.

In the epitope having the sequence of SEQ ID NO: 781, sites of amino acids other than X in XXXXXXXXRXRRXXX or SEQ ID NO: 780 are important for binding to IgE antibodies from allergic patients. Thus, in another preferred mode, amino acids 475-489 of SEQ ID NO: 341 in the antigen in spot 19α may have the amino acid sequence of XXXXXXXXRXRRXXX or SEQ ID NO: 780.

In the epitope having the sequence of SEQ ID NO: 784, sites of amino acids other than X in XXXXXXXXXXEEEXX or SEQ ID NO: 783 are important for binding to IgE antibodies from allergic patients. Thus, in another preferred mode, amino acids 81-95 of SEQ ID NO: 341 in the antigen in spot 19α may have the amino acid sequence of XXXXXXXXXXEEEXX or SEQ ID NO: 783.

Preferably, a protein that is an antigen in spot 19α has activity equivalent to that of the protein having the amino acid sequence of SEQ ID NO: 341 (sucrose binding protein homolog S-64) or causative of an allergy to soybean.

### (25α) Antigen in Spot 25α

As the result of sequence identification of the antigen in spot 25α by mass spectroscopy, the amino acid sequences of SEQ ID NOs: 349-365 were detected.

Also, the mass spectroscopic data obtained for spot 25α on a mass spectrometer was analyzed by comparing the data against the NCBI protein data, and as a result, succinyl-CoA ligase [ADP-forming] subunit beta, mitochondrial (amino acid sequence: SEQ ID NO: 348, encoding nucleotide sequence: SEQ ID NO: 347) was identified as a protein comprising any of SEQ ID NOs: 349 to 356 and succinyl-CoA ligase [ADP-forming] subunit beta, mitochondrial (amino acid sequence: SEQ ID NO: 358, encoding nucleotide sequence: SEQ ID NO: 357) was identified as a protein comprising any of SEQ ID NOs: 359 to 365.

Accordingly, the antigen in spot 25α in the present invention can be any of proteins selected from the group consisting of the proteins as defined below in (25αA1-a) to (25αA1-c), (25αB1), (25αA2-a) to (25αA2-c), and (25αB2):
(25αA1-a) a protein comprising an amino acid sequence with deletion, substitution, insertion or addition of one or several amino acids in SEQ ID NO: 348;
(25αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 348;
(25αA1-c) a protein comprising an amino acid sequence encoded by a nucleotide sequence with deletion, substitution, insertion or addition of one or several nucleotides in SEQ ID NO: 347;
(25αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 347;
(25αA1-e) a protein comprising an amino acid sequence encoded by a nucleic acid that hybridizes under stringent conditions with a nucleic acid having a nucleotide sequence complementary to the nucleotide sequence of SEQ ID NO: 347;
(25αB1) a protein comprising at least one amino acid sequence selected from the group consisting of SEQ ID NOs: 349-356, preferably a protein comprising at least 2, 3, 4, 5, 6, or 7 or all sequences of the amino acid sequences. As referred to above, the amino acid sequence of any of SEQ ID NOs: 349 to 356 may be an amino acid sequence derived therefrom by deletion, substitution, insertion or addition of one or several amino acids;
(25αA2-a) a protein comprising an amino acid sequence with deletion, substitution, insertion or addition of one or several amino acids in SEQ ID NO: 358;
(25αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 358;
(25αA2-c) a protein comprising an amino acid sequence encoded by a nucleotide sequence with deletion, substitution, insertion or addition of one or several nucleotides in SEQ ID NO: 357;
(25αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 357;
(25αA2-c) a protein comprising an amino acid sequence encoded by a nucleic acid that hybridizes under stringent conditions with a nucleic acid having a nucleotide sequence complementary to the nucleotide sequence of SEQ ID NO: 357;
(25% B2) a protein comprising at least one amino acid sequence selected from the group consisting of SEQ ID NOs: 359-365, preferably a protein comprising at least 2, 3, 4, 5, or 6 or all sequences of the amino acid sequences. As referred to above, the amino acid sequence of any of SEQ ID NOs: 359 to 365 may be an amino acid sequence derived therefrom by deletion, substitution, insertion or addition of one or several amino acids.

The protein selected from the group consisting of the proteins as defined above in (25αA1-a) to (25αA1-c), (25αB1), (25αA2-a) to (25αA2-c), and (25αB2) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (25αA1-a) to (25αA1-c) and (25αB1) reside in amino acids 241-255 (SEQ ID NO: 814) and amino acids 321-335 (SEQ ID NO: 815) of SEQ ID NO: 348. When the antigen in spot 25α contains a variation in the amino acid sequence of SEQ ID NO: 348, this antigen may retain the sequence in SEQ ID NO: 348 as to amino acids at positions corresponding to the region of at least one of these epitopes.

Preferably, a protein that is an antigen in spot 25α has activity equivalent to that of the protein having the amino acid sequence of SEQ ID NO: 348 or 358 (succinyl-CoA ligase [ADP-forming] subunit beta, mitochondrial) or causative of an allergy to soybean.

### (26α) Antigen in Spot 26α

As the result of sequence identification of the antigen in spot 26α by mass spectroscopy, the amino acid sequences of SEQ ID NOs: 368-409 were detected.

Also, the mass spectroscopic data obtained for spot 26α on a mass spectrometer was analyzed by comparing the data against the NCBI protein data, and as a result, phosphoglycerate kinase, cytosolic (amino acid sequence: SEQ ID NO: 367, encoding nucleotide sequence: SEQ ID NO: 366) was identified as a protein comprising any of SEQ ID NOs: 368 to 382, phosphoglycerate kinase, cytosolic (amino acid sequence: SEQ ID NO: 384, encoding nucleotide sequence: SEQ ID NO: 383) was identified as a protein comprising any of SEQ ID NOs: 385 to 392, and phosphoglycerate kinase, cytosolic (amino acid sequence: SEQ ID NO: 394, encoding nucleotide sequence: SEQ ID NO: 393) was identified as a protein comprising any of SEQ ID NOs: 395 to 409.

Accordingly, the antigen in spot 26α in the present invention can be any of proteins selected from the group consisting of the proteins as defined below in (26αA1-a) to (26αA1-c), (26αB1), (26αA2-a) to (26αA2-c), (26αB2), (26αA3-a) to (26αA3-c), and (26αB3):
(26αA1-a) a protein comprising an amino acid sequence with deletion, substitution, insertion or addition of one or several amino acids in SEQ ID NO: 367;
(26αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 367;
(26αA1-c) a protein comprising an amino acid sequence encoded by a nucleotide sequence with deletion, substitution, insertion or addition of one or several nucleotides in SEQ ID NO: 366;
(26αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 366;
(26αA1-c) a protein comprising an amino acid sequence encoded by a nucleic acid that hybridizes under stringent conditions with a nucleic acid having a nucleotide sequence complementary to the nucleotide sequence of SEQ ID NO: 366;
(26αB1) a protein comprising at least one amino acid sequence selected from the group consisting of SEQ ID NOs: 368-382, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 368 to 382 may be an amino acid sequence derived therefrom by deletion, substitution, insertion or addition of one or several amino acids;
(26αA2-a) a protein comprising an amino acid sequence with deletion, substitution, insertion or addition of one or several amino acids in SEQ ID NO: 384;
(26αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 384;
(26αA2-c) a protein comprising an amino acid sequence encoded by a nucleotide sequence with deletion, substitution, insertion or addition of one or several nucleotides in SEQ ID NO: 383;
(26αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 383;
(26αA2-c) a protein comprising an amino acid sequence encoded by a nucleic acid that hybridizes under stringent conditions with a nucleic acid having a nucleotide sequence complementary to the nucleotide sequence of SEQ ID NO: 383;
(26αB2) a protein comprising at least one amino acid sequence selected from the group consisting of SEQ ID NOs: 385-392, preferably a protein comprising at least 2, 3, 4, 5, 6, or 7 or all sequences of the amino acid sequences. As referred to above, the amino acid sequence of any of SEQ ID NOs: 385 to 392 may be an amino acid sequence derived therefrom by deletion, substitution, insertion or addition of one or several amino acids.
(26αA3-a) a protein comprising an amino acid sequence with deletion, substitution, insertion or addition of one or several amino acids in SEQ ID NO: 394;
(26αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 394;
(26αA3-c) a protein comprising an amino acid sequence encoded by a nucleotide sequence with deletion, substitution, insertion or addition of one or several nucleotides in SEQ ID NO: 393;
(26αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 393;
(26αA3-c) a protein comprising an amino acid sequence encoded by a nucleic acid that hybridizes under stringent conditions with a nucleic acid having a nucleotide sequence complementary to the nucleotide sequence of SEQ ID NO: 393;
(26αB3) a protein comprising at least one amino acid sequence selected from the group consisting of SEQ ID NOs: 395-409, preferably a protein comprising at least 2, 3, 4, 5, 6, 7, 8, 9, 10, 11, 12, 13, or 14 or all sequences of the amino acid sequences. As referred to above, the amino acid sequence of any of SEQ ID NOs: 395 to 409 may be an amino acid sequence derived therefrom by deletion, substitution, insertion or addition of one or several amino acids.

The protein selected from the group consisting of the proteins as defined above in (26αA1-a) to (26αA1-c), (26αB1), (26αA2-a) to (26αA2-c), (26αB2), (26αA3-a) to (26αA3-c), and (26αB3) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”. Epitopes of the aforementioned antigens (26αA1-a) to (26αA1-c) and (26αB1) reside in amino acids 121-135 (SEQ ID NO: 816) and amino acids 329-335 (SEQ ID NO: 819) of SEQ ID NO: 367. When the antigen in spot 26α contains a variation in the amino acid sequence of SEQ ID NO: 367, this antigen may retain the sequence in SEQ ID NO: 361 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 26α may be the following variant.

In the epitope having the sequence of SEQ ID NO: 819, sites of amino acids other than X in FDKXXXX or SEQ ID NO: 818 are important for binding to IgE antibodies from allergic patients. Thus, in another preferred mode, when the antigen in spot 26α contains a variation in the amino acid sequence of SEQ ID NO: 367, amino acids 329-335 of SEQ ID NO: 367 in this antigen may have the amino acid sequence of FDKXXXX or SEQ ID NO: 818.

Preferably, a protein that is an antigen in spot 25α has activity equivalent to that of the protein having the amino acid sequence of SEQ ID NO: 367, 384, or 394 (phosphoglycerate kinase, cytosolic) or causative of an allergy to soybean.

### (27α) Antigen in Spot 27α

As the result of sequence identification of the antigen in spot 27α by mass spectroscopy, the amino acid sequences of SEQ ID NOs: 412-427 were detected.

Also, the mass spectroscopic data obtained for spot 27a on a mass spectrometer was analyzed by comparing the data against the NCBI protein data, and as a result, alcohol dehydrogenase 1 (amino acid sequence: SEQ ID NO: 411, encoding nucleotide sequence: SEQ ID NO: 410) was identified as a protein comprising any of SEQ ID NOs: 412 to 416, alcohol dehydrogenase 1 (amino acid sequence: SEQ ID NO: 418, encoding nucleotide sequence: SEQ ID NO: 417) was identified as a protein comprising any of SEQ ID NOs: 419 and 420, and alcohol dehydrogenase 1-like (amino acid sequence: SEQ ID NO: 422, encoding nucleotide sequence: SEQ ID NO: 421) was identified as a protein comprising any of SEQ ID NOs: 423 to 427.

Accordingly, the antigen in spot 27 in the present invention can be any of proteins selected from the group consisting of the proteins as defined below in (27αA1-a) to (27αA1-e), (27αB1), (27αA2-a) to (27αA2-c), (27αB2), (27αA3-a) to (27αA3-c), and (27αB3): (27αA1-a) a protein comprising an amino acid sequence with deletion, substitution, insertion or addition of one or several amino acids in SEQ ID NO: 411;
(27αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 411;
(27αA1-c) a protein comprising an amino acid sequence encoded by a nucleotide sequence with deletion, substitution, insertion or addition of one or several nucleotides in SEQ ID NO: 410;
(27αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 410;
(27αA1-e) a protein comprising an amino acid sequence encoded by a nucleic acid that hybridizes under stringent conditions with a nucleic acid having a nucleotide sequence complementary to the nucleotide sequence of SEQ ID NO: 410;
(27αB1) a protein comprising at least one amino acid sequence selected from the group consisting of SEQ ID NOs: 412-416, preferably a protein comprising at least 2, 3, or 4 or all sequences of the amino acid sequences. As referred to above, the amino acid sequence of any of SEQ ID NOs: 412 to 416 may be an amino acid sequence derived therefrom by deletion, substitution, insertion or addition of one or several amino acids;
(27αA2-a) a protein comprising an amino acid sequence with deletion, substitution, insertion or addition of one or several amino acids in SEQ ID NO: 418;
(27αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 418;
(27αA2-c) a protein comprising an amino acid sequence encoded by a nucleotide sequence with deletion, substitution, insertion or addition of one or several nucleotides in SEQ ID NO: 417;
(27αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 417;
(27αA2-c) a protein comprising an amino acid sequence encoded by a nucleic acid that hybridizes under stringent conditions with a nucleic acid having a nucleotide sequence complementary to the nucleotide sequence of SEQ ID NO: 417;
(27αB2) a protein comprising at least one amino acid sequence selected from the group consisting of SEQ ID NOs: 419 and 420, preferably a protein comprising both the amino acid sequences. As referred to above, the amino acid sequence of any of SEQ ID NOs: 419, 420 may be an amino acid sequence derived therefrom by deletion, substitution, insertion or addition of one or several amino acids.
(27αA3-a) a protein comprising an amino acid sequence with deletion, substitution, insertion or addition of one or several amino acids in SEQ ID NO: 422;
(27αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 422;
(27αA3-c) a protein comprising an amino acid sequence encoded by a nucleotide sequence with deletion, substitution, insertion or addition of one or several nucleotides in SEQ ID NO: 421; (27αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 421;
(27αA3-c) a protein comprising an amino acid sequence encoded by a nucleic acid that hybridizes under stringent conditions with a nucleic acid having a nucleotide sequence complementary to the nucleotide sequence of SEQ ID NO: 421;
(27αB3) a protein comprising at least one amino acid sequence selected from the group consisting of SEQ ID NOs: 423-427, preferably a protein comprising at least 2, 3, or 4 or all sequences of the amino acid sequences. As referred to above, the amino acid sequence of any of SEQ ID NOs: 423 to 427 may be an amino acid sequence derived therefrom by deletion, substitution, insertion or addition of one or several amino acids.

The protein selected from the group consisting of the proteins as defined above in (27αA1-a) to (27αA1-c), (27αB1), (27αA2-a) to (27αA2-c), (27αB2), (27αA3-a) to (27αA3-c), and (27αB3) can be proteins that are found in a protein spot with a molecular weight of around 20 to 90 kDa, preferably around 30 to 80 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (27αA1-a) to (27αA1-e) and (27αB1) reside in amino acids 21-35 (SEQ ID NO: 820) of SEQ ID NO: 411. When the antigen in spot 27α contains a variation in the amino acid sequence of SEQ ID NO: 411, this antigen may retain the sequence in SEQ ID NO: 411 as to amino acids at positions corresponding to the region of this epitope.

Preferably, a protein that is an antigen in spot 27α has activity equivalent to that of the protein having the amino acid sequence of SEQ ID NO: 411 or 418 (alcohol dehydrogenase 1) or the protein having the amino acid sequence of SEQ ID NO: 422 (alcohol dehydrogenase 1-like) or causative of an allergy to soybean.

### (28α) Antigen in Spot 28α

As the result of sequence identification of the antigen in spot 28α by mass spectroscopy, the amino acid sequences of SEQ ID NOs: 430-433 were detected.

Also, the mass spectroscopic data obtained for spot 28α on a mass spectrometer was analyzed by comparing the data against the NCBI protein data, and as a result, 35 kDa seed maturation protein isoform X1 (amino acid sequence: SEQ ID NO: 429, encoding nucleotide sequence: SEQ ID NO: 428) was identified as a protein comprising any of SEQ ID NO: 430 to 433.

Accordingly, the antigens in spot 28α in the present invention can be any protein selected from the group consisting of the proteins as defined below in (28αA-a) to (28αA-e) and (28αB):
(28αA-a) a protein comprising an amino acid sequence with deletion, substitution, insertion or addition of one or several amino acids in SEQ ID NO: 429;
(28αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 429;
(28αA-c) a protein comprising an amino acid sequence encoded by a nucleotide sequence with deletion, substitution, insertion or addition of one or several nucleotides in SEQ ID NO: 428;
(28αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 428;
(28αA-e) a protein comprising an amino acid sequence encoded by a nucleic acid that hybridizes under stringent conditions with a nucleic acid having a nucleotide sequence complementary to the nucleotide sequence of SEQ ID NO: 428;
(28αB) a protein comprising at least one amino acid sequence selected from the group consisting of SEQ ID NOs: 430-433, preferably a protein comprising at least 2 or 3 or all sequences of the amino acid sequences. As referred to above, the amino acid sequence of any of SEQ ID NOs: 430-433 may be an amino acid sequence derived therefrom by deletion, substitution, insertion or addition of one or several amino acids.

The proteins selected from the group consisting of (28αA-a) to (28αA-c) and (28β) as defined above can be proteins that are found in a protein spot with a molecular weight of around 10 to 70 kDa, preferably around 20 to 60 kDa and an isoelectric point of 3.5 to 7.5, preferably 4.0 to 7.0 on gels used in the two-dimensional electrophoresis performed under the conditions described above in the subsection titled “Identification of antigens”.

Epitopes of the antigen in spot 28α reside in amino acids 76-85 (SEQ ID NO: 820), amino acids 191-205 (SEQ ID NO: 826), amino acids 231-245 (SEQ ID NO: 829), amino acids 111-125 (SEQ ID NO: 830), amino acids 131-145 (SEQ ID NO: 831), amino acids 161-175 (SEQ ID NO: 832), amino acids 181-195 (SEQ ID NO: 833), amino acids 291-305 (SEQ ID NO: 834), amino acids 295-309 (SEQ ID NO: 835) of SEQ ID NO: 429. The antigen in spot 28a may retain the sequence in SEQ ID NO: 429 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 28α may be the following variant.

In the epitope having the sequence of SEQ ID NO: 823, sites of amino acids other than X in SEQ ID NO: 821 or 822 are important for binding to IgE antibodies from allergic patients. Thus, in another preferred mode, amino acids 76-85 of SEQ ID NO: 429 in the antigen in spot 28a may have the amino acid sequence of SEQ ID NO: 821 or 822.

In the epitope having the sequence of SEQ ID NO: 826, sites of amino acids other than X in XKXXXXEKXXXXXXX or SEQ ID NO: 825 are important for binding to IgE antibodies from allergic patients. Thus, in another preferred mode, amino acids 191-205 of SEQ ID NO: 429 in the antigen in spot 28α may have the amino acid sequence of XKXXXXEKXXXXXXX or SEQ ID NO: 825.

In the epitope having the sequence of SEQ ID NO: 829, sites of amino acids other than X in SEQ ID NO: 827 or 828 are important for binding to IgE antibodies from allergic patients. Thus, in another preferred mode, amino acids 231-2455 of SEQ ID NO: 429 in the antigen in spot 28α may have the amino acid sequence of SEQ ID NO: 827 or 828.

Preferably, a protein that is an antigen in spot 28α has activity equivalent to that of the protein having the amino acid sequence of SEQ ID NO: 422 (35 kDa seed maturation protein isoform X1) or causative of an allergy to soybean.

### (29α, 30α, 31α, 32α, 33α) Antigens in Spots 29α to 33α

As the result of sequence identification of the antigens in spots 29α to 33a by mass spectroscopy, the amino acid sequences of SEQ ID NOs: 436-451 were detected.

Also, the mass spectroscopic data obtained for spots 29a to 33a on a mass spectrometer was analyzed by comparing the data against the NCBI protein data, and as a result, glyceraldehyde-3-phosphate dehydrogenase isoform X1 (amino acid sequence: SEQ ID NO: 435, encoding nucleotide sequence: SEQ ID NO: 434) was identified as a protein comprising any of SEQ ID NO: 436 to 439, glyceraldehyde-3-phosphate dehydrogenase, cytosolic (amino acid sequence: SEQ ID NO: 441, encoding nucleotide sequence: SEQ ID NO: 440) was identified as a protein comprising any of SEQ ID NO: 442 to 445, and uncharacterized protein LOC100782924 (amino acid sequence: SEQ ID NO: 447, encoding nucleotide sequence: SEQ ID NO: 446) was identified as a protein comprising any of SEQ ID NO: 448 to 451.

Accordingly, the antigen in spot 29α to 33α in the present invention can be any of proteins selected from the group consisting of the proteins as defined below in (29-33αA1-a) to (29-33αA1-c), (29-33αB1), (29-33αA2-a) to (29-33αA2-c), (29-33αB2), (29-33αA3-a) to (29-33αA3-c), and (29-33αB3):
(29-33αA1-a) a protein comprising an amino acid sequence with deletion, substitution, insertion or addition of one or several amino acids in SEQ ID NO: 435;
(29-33αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 435;
(29-33αA1-c) a protein comprising an amino acid sequence encoded by a nucleotide sequence with deletion, substitution, insertion or addition of one or several nucleotides in SEQ ID NO: 434;
(29-33αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 434;
(29-33αA1-e) a protein comprising an amino acid sequence encoded by a nucleic acid that hybridizes under stringent conditions with a nucleic acid having a nucleotide sequence complementary to the nucleotide sequence of SEQ ID NO: 434;
(29-33αB1) a protein comprising at least one amino acid sequence selected from the group consisting of SEQ ID NOs: 436-439, preferably a protein comprising at least 2, 3, or all sequences of the amino acid sequences. As referred to above, the amino acid sequence of any of SEQ ID NOs: 436 to 439 may be an amino acid sequence derived therefrom by deletion, substitution, insertion or addition of one or several amino acids;
(29-33αA2-a) a protein comprising an amino acid sequence with deletion, substitution, insertion or addition of one or several amino acids in SEQ ID NO: 441;
(29-33αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 441;
(29-33αA2-c) a protein comprising an amino acid sequence encoded by a nucleotide sequence with deletion, substitution, insertion or addition of one or several nucleotides in SEQ ID NO: 440;
(29-33αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 440;
(29-33αA2-c) a protein comprising an amino acid sequence encoded by a nucleic acid that hybridizes under stringent conditions with a nucleic acid having a nucleotide sequence complementary to the nucleotide sequence of SEQ ID NO: 440;
(29-33αB2) a protein comprising at least one amino acid sequence selected from the group consisting of SEQ ID NOs: 442-445, preferably a protein comprising at least 2, 3, or all sequences of the amino acid sequences. As referred to above, the amino acid sequence of any of SEQ ID NOs: 442 to 445 may be an amino acid sequence derived therefrom by deletion, substitution, insertion or addition of one or several amino acids.
(29-33αA3-a) a protein comprising an amino acid sequence with deletion, substitution, insertion or addition of one or several amino acids in SEQ ID NO: 447;
(29-33αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 447;
(29-33αA3-c) a protein comprising an amino acid sequence encoded by a nucleotide sequence with deletion, substitution, insertion or addition of one or several nucleotides in SEQ ID NO: 446;
(29-33αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 446;
(29-33αA3-e) a protein comprising an amino acid sequence encoded by a nucleic acid that hybridizes under stringent conditions with a nucleic acid having a nucleotide sequence complementary to the nucleotide sequence of SEQ ID NO: 446;
(29-33αB3) a protein comprising at least one amino acid sequence selected from the group consisting of SEQ ID NOs: 448-451, preferably a protein comprising at least 2, 3, or all sequences of the amino acid sequences. As referred to above, the amino acid sequence of any of SEQ ID NOs: 448 to 451 may be an amino acid sequence derived therefrom by deletion, substitution, insertion or addition of one or several amino acids.

The protein selected from the group consisting of the proteins as defined above in (29-33αA1-a) to (29-33αA1-c), (29-33αB1), (29-33αA2-a) to (29-33αA2-c), (29-33αB2), (29-33αA3-a) to (29-33αA3-c), and (29-33αB3) can be proteins that are found in a protein spot with a molecular weight of around 10 to 70 kDa, preferably around 20 to 60 kDa, and an isoelectric point of 3.5 to 8.5, preferably 4.0 to 8.0 on gels used in the two-dimensional electrophoresis performed under the conditions described above in the subsection titled “Identification of antigens”.

Epitopes of the aforementioned antigens (29-33αA1-a) to (29-33αA1-c) and (29-33αB1) reside in amino acids 66-75 (SEQ ID NO: 838), amino acids 301-315 (SEQ ID NO: 839) of SEQ ID NO: 435. When the antigen in spot 29-33α contains a variation in the amino acid sequence of SEQ ID NO: 435, this antigen may retain the sequence in SEQ ID NO: 435 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 29-33α may be the following variant.

In the epitope having the sequence of SEQ ID NO: 838, sites of amino acids other than X in XXXXXXXDKP or SEQ ID NO: 837 are important for binding to IgE antibodies from allergic patients. Thus, in another preferred mode, when the antigen in spot 29-33α contains a variation in the amino acid sequence of SEQ ID NO: 435, amino acids 66-75 of SEQ ID NO: 435 in this antigen may have the amino acid sequence of XXXXXXXDKP or SEQ ID NO: 837.

Preferably, a protein that is an antigen in any of spots 29α to 33α has activity equivalent to that of the protein having the amino acid sequence of SEQ ID NO: 435 (glyceraldehyde-3-phosphate dehydrogenase isoform X1), the protein having the amino acid sequence of SEQ ID NO: 441 (glyceraldehyde-3-phosphate dehydrogenase, cytosolic), or the protein having the amino acid sequence of SEQ ID NO: 447 (uncharacterized protein LOC100782924) or causative of an allergy to soybean.

### (34α) Antigen in Spot 34α

As the result of sequence identification of the antigen in spot 34α by mass spectroscopy, the amino acid sequences of SEQ ID NOs: 454-458 were detected.

Also, the mass spectroscopic data obtained for spot 34α on a mass spectrometer was analyzed by comparing the data against the NCBI protein data, and as a result, bifunctional UDP-glucose 4-epimerase and UDP-xylose 4-epimerase 1-like (amino acid sequence: SEQ ID NO: 453, encoding nucleotide sequence: SEQ ID NO: 452) was identified as a protein comprising SEQ ID NO: 454 and uncharacterized protein LOC100803483 (amino acid sequence: SEQ ID NO: 456, encoding nucleotide sequence: SEQ ID NO: 455) was identified as a protein comprising SEQ ID NO: 457 or 458.

Accordingly, the antigen in spot 34α in the present invention can be any of proteins selected from the group consisting of the proteins as defined below in (34αA1-a) to (34αA1-c), (34αB1), (34αA2-a) to (34αA2-c), and (34αB2):
(34αA1-a) a protein comprising an amino acid sequence with deletion, substitution, insertion or addition of one or several amino acids in SEQ ID NO: 453;
(34αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 453;
(34αA1-c) a protein comprising an amino acid sequence encoded by a nucleotide sequence with deletion, substitution, insertion or addition of one or several nucleotides in SEQ ID NO: 452;
(34αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 452;
(34αA1-c) a protein comprising an amino acid sequence encoded by a nucleic acid that hybridizes under stringent conditions with a nucleic acid having a nucleotide sequence complementary to the nucleotide sequence of SEQ ID NO: 452;
(34αB1) a protein comprising an amino acid sequence of SEQ ID NOs: 454. As referred to above, the amino acid sequence of any of SEQ ID NOs: 464 may be an amino acid sequence derived therefrom by deletion, substitution, insertion or addition of one or several amino acids;
(34αA2-a) a protein comprising an amino acid sequence with deletion, substitution, insertion or addition of one or several amino acids in SEQ ID NO: 456;
(34αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 456;
(34αA2-c) a protein comprising an amino acid sequence encoded by a nucleotide sequence with deletion, substitution, insertion or addition of one or several nucleotides in SEQ ID NO: 455;
(34αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 455;
(34αA2-c) a protein comprising an amino acid sequence encoded by a nucleic acid that hybridizes under stringent conditions with a nucleic acid having a nucleotide sequence complementary to the nucleotide sequence of SEQ ID NO: 455;
(34αB2) a protein comprising at least one amino acid sequence selected from the group consisting of SEQ ID NOs: 457 and 458, preferably a protein comprising both the amino acid sequences. As referred to above, the amino acid sequence of any of SEQ ID NOs: 457, 458 may be an amino acid sequence derived therefrom by deletion, substitution, insertion or addition of one or several amino acids.

The protein selected from the group consisting of the proteins as defined above in (34αA1-a) to (34αA1-c), (34αB1), (34αA2-a) to (34αA2-c), and (34αB2) can be proteins that are found in a protein spot with a molecular weight of around 10 to 70 kDa, preferably around 20 to 60 kDa, and an isoelectric point of 5.5 to 10.5, preferably 6.0 to 10.0 on gels used in the two-dimensional electrophoresis performed under the conditions described above in the subsection titled “Identification of antigens”.

Epitopes of the aforementioned antigens (34αA1-a) to (34αA1-c) and (34αB1) reside in amino acids 61-75 (SEQ ID NO: 842), amino acids 31-45 (SEQ ID NO: 843), amino acids 141-155 (SEQ ID NO: 844), amino acids 221-235 (SEQ ID NO: 845), amino acids 231-245 (SEQ ID NO: 846) of SEQ ID NO: 454. When the antigen in spot 34α contains a variation in the amino acid sequence of SEQ ID NO: 454, this antigen may retain the sequence in SEQ ID NO: 454 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 34α may be the following variant.

In the epitope having the sequence of SEQ ID NO: 842, sites of amino acids other than X in XXXXXXXXXXXDDXE or SEQ ID NO: 841 are important for binding to IgE antibodies from allergic patients. Thus, in another preferred mode, when the antigen in spot 34α contains a variation in the amino acid sequence of SEQ ID NO: 454, amino acids 61-75 of SEQ ID NO: 454 in this antigen may have the amino acid sequence of XXXXXXXXXXXDDXE or SEQ ID NO: 841.

Preferably, a protein that is an antigen in spot 34α has activity equivalent to that of the protein having the amino acid sequence of SEQ ID NO: 453 (bifunctional UDP-glucose 4-epimerase and UDP-xylose 4-epimerase 1-like) or the protein having the amino acid sequence of SEQ ID NO: 456 (uncharacterized protein LOC100803483) or causative of an allergy to soybean.

### (35α) Antigen in Spot 35α

As the result of sequence identification of the antigen in spot 35α by mass spectroscopy, the amino acid sequences of SEQ ID NOs: 461-477 were detected.

Also, the mass spectroscopic data obtained for spot 35α on a mass spectrometer was analyzed by comparing the data against the NCBI protein data, and as a result, elongation factor 1-alpha (amino acid sequence: SEQ ID NO: 460, encoding nucleotide sequence: SEQ ID NO: 459) was identified as a protein comprising any of SEQ ID NOs: 461 to 466, elongation factor 1-alpha (amino acid sequence: SEQ ID NO: 468, encoding nucleotide sequence: SEQ ID NO: 467) was identified as a protein comprising any of SEQ ID NOs: 469 to 471, and elongation factor-1A isoform X1 (amino acid sequence: SEQ ID NO: 473, encoding nucleotide sequence: SEQ ID NO: 472) was identified as a protein comprising any of SEQ ID NOs: 474 to 477.

Accordingly, the antigen in spot 35α in the present invention can be any of proteins selected from the group consisting of the proteins as defined below in (35αA1-a) to (35QAl-c), (35αB1), (35αA2-a) to (35αA2-c), (35αB2), (35αA3-a) to (35αA3-c), and (35αB3): (35αA1-a) a protein comprising an amino acid sequence with deletion, substitution, insertion or addition of one or several amino acids in SEQ ID NO: 460;
(35αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 460;
(35αA1-c) a protein comprising an amino acid sequence encoded by a nucleotide sequence with deletion, substitution, insertion or addition of one or several nucleotides in SEQ ID NO: 459;
(35αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 459;
(35αA1-e) a protein comprising an amino acid sequence encoded by a nucleic acid that hybridizes under stringent conditions with a nucleic acid having a nucleotide sequence complementary to the nucleotide sequence of SEQ ID NO: 459;
(35αB1) a protein comprising at least one amino acid sequence selected from the group consisting of SEQ ID NOs: 461-466, preferably a protein comprising at least 2, 3, 4, or 5 or all sequences of the amino acid sequences. As referred to above, the amino acid sequence of any of SEQ ID NOs: 412 to 416 may be an amino acid sequence derived therefrom by deletion, substitution, insertion or addition of one or several amino acids;
(35αA2-a) a protein comprising an amino acid sequence with deletion, substitution, insertion or addition of one or several amino acids in SEQ ID NO: 468;
(35αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 468;
(35αA2-c) a protein comprising an amino acid sequence encoded by a nucleotide sequence with deletion, substitution, insertion or addition of one or several nucleotides in SEQ ID NO: 467;
(35αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 467;
(35αA2-c) a protein comprising an amino acid sequence encoded by a nucleic acid that hybridizes under stringent conditions with a nucleic acid having a nucleotide sequence complementary to the nucleotide sequence of SEQ ID NO: 467;
(35αB2) a protein comprising at least one amino acid sequence selected from the group consisting of SEQ ID NOs: 469-471, preferably a protein comprising at least 2, or all sequences of the amino acid sequences. As referred to above, the amino acid sequence of any of SEQ ID NOs: 469 to 471 may be an amino acid sequence derived therefrom by deletion, substitution, insertion or addition of one or several amino acids.
(35αA3-a) a protein comprising an amino acid sequence with deletion, substitution, insertion or addition of one or several amino acids in SEQ ID NO: 473;
(35αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 473;
(35αA3-c) a protein comprising an amino acid sequence encoded by a nucleotide sequence with deletion, substitution, insertion or addition of one or several nucleotides in SEQ ID NO: 472;
(35αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 472;
(35αA3-c) a protein comprising an amino acid sequence encoded by a nucleic acid that hybridizes under stringent conditions with a nucleic acid having a nucleotide sequence complementary to the nucleotide sequence of SEQ ID NO: 472;
(35αB3) a protein comprising at least one amino acid sequence selected from the group consisting of SEQ ID NOs: 474-477, preferably a protein comprising at least 2, 3, or all sequences of the amino acid sequences. As referred to above, the amino acid sequence of any of SEQ ID NOs: 474 to 477 may be an amino acid sequence derived therefrom by deletion, substitution, insertion or addition of one or several amino acids.

The protein selected from the group consisting of the proteins as defined above in (35αA1-a) to (35αA1-c), (35αB1), (35αA2-a) to (35αA2-c), (35αB2), (35αA3-a) to (35αA3-c), and (35αB3) can be proteins that are found in a protein spot with a molecular weight of around 20 to 90 kDa, preferably around 30 to 80 kDa, and an isoelectric point of 5.5 to 10.5, preferably 6.0 to 10.0 on gels used in the two-dimensional electrophoresis performed under the conditions described above in the subsection titled “Identification of antigens”.

Epitopes of the aforementioned antigens (35αA1-a) to (35αA1-e) and (35αB1) reside in amino acids 31-45 (SEQ ID NO: 849), amino acids 121-135 (SEQ ID NO: 850), amino acids 192-220 (SEQ ID NO: 853), amino acids 216-225 (SEQ ID NO: 856) of SEQ ID NO: 460. When the antigen in spot 35α contains a variation in the amino acid sequence of SEQ ID NO: 460, amino acids at positions corresponding to the region of at least one of these epitopes may retain the sequence in SEQ ID NO: 460.

From the viewpoint of improving sensitivity (a degree to which a patient can be diagnosed as being positive) or specificity (a degree to which a healthy subject is not diagnosed as being positive), the antigen in spot 35α may be the following variant.

In the epitope having the sequence of SEQ ID NO: 849, sites of amino acids other than X in SEQ ID NO: 847 or 848 are important for binding to IgE antibodies from allergic patients. Thus, in another preferred mode, when the antigen in spot 35α contains a variation in the amino acid sequence of SEQ ID NO: 460, amino acids 31-45 of SEQ ID NO: 460 in this antigen may have the amino acid sequence of SEQ ID NO: 847 or 848.

In the epitope having the sequence of SEQ ID NO: 853, sites of amino acids other than X in SEQ ID NO: 851 or 852 are important for binding to IgE antibodies from allergic patients. Thus, in another preferred mode, when the antigen in spot 35α contains a variation in the amino acid sequence of SEQ ID NO: 460, amino acids 192-220 of SEQ ID NO: 460 in this antigen may have the amino acid sequence of SEQ ID NO: 851 or 852.

In the epitope having the sequence of SEQ ID NO: 856, sites of amino acids other than X in XXEXLDXXXX or SEQ ID NO: 855 are important for binding to IgE antibodies from allergic patients. Thus, in another preferred mode, when the antigen in spot 35α contains a variation in the amino acid sequence of SEQ ID NO: 460, amino acids 216-225 of SEQ ID NO: 460 in this antigen may have the amino acid sequence of XXEXLDXXXX or SEQ ID NO: 855.

Preferably, a protein that is an antigen in spot 35α has activity equivalent to that of the protein having the amino acid sequence of SEQ ID NO: 460 or 468 (elongation factor 1-alpha) or the protein having the amino acid sequence of SEQ ID NO: 473 (elongation factor-1A isoform X1) or causative of an allergy to soybean.

### (36α, 37α) Antigens in Spots 36α, 37α

As the result of sequence identification of the antigens in spots 36α and 37α by mass spectroscopy, the amino acid sequences of SEQ ID NOs: 480-493 were detected.

Also, the mass spectroscopic data obtained for spots 36α and 37a on a mass spectrometer was analyzed by comparing the data against the NCBI protein data, and as a result, seed maturation protein PM34 (amino acid sequence: SEQ ID NO: 479, encoding nucleotide sequence: SEQ ID NO: 478) was identified as a protein comprising any of SEQ ID NOs: 480 to 483, uncharacterized protein LOC100809384 (amino acid sequence: SEQ ID NO: 485, encoding nucleotide sequence: SEQ ID NO: 484) was identified as a protein comprising SEQ ID NO: 486 or 487, and hypothetical protein GLYMA_10G155300 (amino acid sequence: SEQ ID NO: 489, encoding nucleotide sequence: SEQ ID NO: 488) was identified as a protein comprising any of SEQ ID NOs: 4490 to 493.

Accordingly, the antigen in spot 36α, 37a in the present invention can be any of proteins selected from the group consisting of the proteins as defined below in (36, 37αA1-a) to (36, 37αA1-e), (36, 37αB1), (36, 37αA2-a) to (36, 37αA2-e), (36, 37αB2), (36, 37αA3-a) to (36, 37αA3-e), and (36, 37αB3):
(36, 37αA1-a) a protein comprising an amino acid sequence with deletion, substitution, insertion or addition of one or several amino acids in SEQ ID NO: 479;
(36, 37αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 479;
(36, 37αA1-c) a protein comprising an amino acid sequence encoded by a nucleotide sequence with deletion, substitution, insertion or addition of one or several nucleotides in SEQ ID NO: 478;
(36, 37αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 478;
(36, 37αA1-e) a protein comprising an amino acid sequence encoded by a nucleic acid that hybridizes under stringent conditions with a nucleic acid having a nucleotide sequence complementary to the nucleotide sequence of SEQ ID NO: 478;
(36, 37αB1) a protein comprising at least one amino acid sequence selected from the group consisting of SEQ ID NOs: 480-483, preferably a protein comprising at least 2, 3, or all sequences of the amino acid sequences. As referred to above, the amino acid sequence of any of SEQ ID NOs: 480 to 483 may be an amino acid sequence derived therefrom by deletion, substitution, insertion or addition of one or several amino acids;
(36, 37αA2-a) a protein comprising an amino acid sequence with deletion, substitution, insertion or addition of one or several amino acids in SEQ ID NO: 485;
(36, 37αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 485;
(36, 37αA2-c) a protein comprising an amino acid sequence encoded by a nucleotide sequence with deletion, substitution, insertion or addition of one or several nucleotides in SEQ ID NO: 484;
(36, 37αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 484;
(36, 37αA2-e) a protein comprising an amino acid sequence encoded by a nucleic acid that hybridizes under stringent conditions with a nucleic acid having a nucleotide sequence complementary to the nucleotide sequence of SEQ ID NO: 484;
(36, 37αB2) a protein comprising at least one amino acid sequence selected from the group consisting of SEQ ID NOs: 486 and 487, preferably a protein comprising both the amino acid sequences. As referred to above, the amino acid sequence of any of SEQ ID NOs: 486, 487 may be an amino acid sequence derived therefrom by deletion, substitution, insertion or addition of one or several amino acids.
(36, 37αA3-a) a protein comprising an amino acid sequence with deletion, substitution, insertion or addition of one or several amino acids in SEQ ID NO: 489;
(36, 37αA3-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 489;
(36, 37αA3-c) a protein comprising an amino acid sequence encoded by a nucleotide sequence with deletion, substitution, insertion or addition of one or several nucleotides in SEQ ID NO: 488;
(36, 37αA3-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 488;
(36, 37αA3-e) a protein comprising an amino acid sequence encoded by a nucleic acid that hybridizes under stringent conditions with a nucleic acid having a nucleotide sequence complementary to the nucleotide sequence of SEQ ID NO: 488;
(36, 37αB3) a protein comprising at least one amino acid sequence selected from the group consisting of SEQ ID NOs: 490-493, preferably a protein comprising at least 2, 3, or all sequences of the amino acid sequences. As referred to above, the amino acid sequence of any of SEQ ID NOs: 490 to 493 may be an amino acid sequence derived therefrom by deletion, substitution, insertion or addition of one or several amino acids.

The protein selected from the group consisting of the proteins as defined above in (36, 37αA1-a) to (36, 37αA1-c), (36, 37αB1), (36, 37αA2-a) to (36, 37αA2-c), (36, 37αB2), (36, 37αA3-a) to (36, 37αA3-c), and (36, 37αB3) can be proteins that are found in a protein spot with a molecular weight of around 10 to 70 kDa, preferably around 20 to 60 kDa, and an isoelectric point of 3.5 to 8.5, preferably 4.0 to 8.0 on gels used in the two-dimensional electrophoresis performed under the conditions described above in the subsection titled “Identification of antigens”.

Epitopes of the aforementioned antigens (36, 37αA1-a) to (36, 37αA1-c) and (36, 37αB1) reside in amino acids 141-155 (SEQ ID NO: 857), amino acids 231-245 (SEQ ID NO: 858), amino acids 7-14 (SEQ ID NO: 861) of SEQ ID NO: 479. When the antigen in spot 36α, 37a contains a variation in the amino acid sequence of SEQ ID NO: 479, this antigen may retain the sequence in SEQ ID NO: 479 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 36α,37a may be the following variant.

In the epitope having the sequence of SEQ ID NO: 861, sites of amino acids other than X in SEQ ID NO: 859 or 860 are important for binding to IgE antibodies from allergic patients. Thus, in another preferred mode, when the antigen in spot 36α,37a contains a variation in the amino acid sequence of SEQ ID NO: 479, amino acids 7-14 of SEQ ID NO: 479 in this antigen may have the amino acid sequence of SEQ ID NO: 859 or 860.

Preferably, a protein that is an antigen in spot 36α or 37a has activity equivalent to that of the protein having the amino acid sequence of SEQ ID NO: 479 (seed maturation protein PM34), the protein having the amino acid sequence of SEQ ID NO: 485 (uncharacterized protein LOC100809384), or the protein having the amino acid sequence of SEQ ID NO: 489 (hypothetical protein GLYMA_10G155300) or causative of an allergy to soybean.

### (38) Antigen in Spot 38α

As the result of sequence identification of the antigen in spot 38α by mass spectroscopy, the amino acid sequences of SEQ ID NOs: 496-513 were detected.

Also, the mass spectroscopic data obtained for spot 38α on a mass spectrometer was analyzed by comparing the data against the NCBI protein data, and as a result, seed biotin-containing protein SBP65-like isoform X1 (amino acid sequence: SEQ ID NO: 495, encoding nucleotide sequence: SEQ ID NO: 494) was identified as a protein comprising any of SEQ ID NOs: 496 to 513.

Accordingly, the antigens in spot 38α in the present invention can be any protein selected from the group consisting of the proteins as defined below in (38αA-a) to (38αA-c) and (38«B):
(38αA-a) a protein comprising an amino acid sequence with deletion, substitution, insertion or addition of one or several amino acids in SEQ ID NO: 495;
(38αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 495;
(38αA-c) a protein comprising an amino acid sequence encoded by a nucleotide sequence with deletion, substitution, insertion or addition of one or several nucleotides in SEQ ID NO: 494;
(38αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 494;
(38αA-c) a protein comprising an amino acid sequence encoded by a nucleic acid that hybridizes under stringent conditions with a nucleic acid having a nucleotide sequence complementary to the nucleotide sequence of SEQ ID NO: 494;
(38αB) a protein comprising at least one amino acid sequence selected from the group consisting of SEQ ID NOs: 496-513, preferably a protein comprising at least 2, 3, 4, 5, 6, 7, 8, 9, 10, 11, 12, 13, 14, 15, 16 or 17 or all sequences of the amino acid sequences. As referred to above, the amino acid sequence of any of SEQ ID NOs: 496-513 may be an amino acid sequence derived therefrom by deletion, substitution, insertion or addition of one or several amino acids.

The proteins selected from the group consisting of (38αA-a) to (38αA-c) and (38αB) as defined above can be proteins that are found in a protein spot with a molecular weight of around 70 to 170 kDa, preferably around 80 to 160 kDa and an isoelectric point of 5.5 to 10.5, preferably 6.0 to 10.0 on gels used in the two-dimensional electrophoresis performed under the conditions described above in the subsection titled “Identification of antigens”.

Epitopes of the antigen in spot 38α reside in amino acids 440-446 or 574-580 (SEQ ID NO: 864), amino acids 71-80 (SEQ ID NO: 867), amino acids 521-535 (SEQ ID NO: 869), amino acids 1-15 (SEQ ID NO: 871), amino acids 31-45 (SEQ ID NO: 874), amino acids 221-235 (SEQ ID NO: 877), and amino acids 461-470 (SEQ ID NO: 882), and amino acids 481-490 (SEQ ID NO: 884), and amino acids 821-835 (SEQ ID NO: 887), and amino acids 861-870 (SEQ ID NO: 889), and amino acids 1031-1040 (SEQ ID NO: 892) of SEQ ID NO: 495. The antigen in spot 38α may retain the sequence in SEQ ID NO: 495 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 38α may be the following variant.

In the epitope having the sequence of SEQ ID NO: 864, sites of amino acids other than X in RGKXXXX are important for binding to IgE antibodies from allergic patients. Thus, in another preferred mode, amino acids 440-446 and/or 574-580 of SEQ ID NO: 495 in the antigen in spot 38α may have the amino acid sequence of RGKXXXX.

In the epitope having the sequence of SEQ ID NO: 867, sites of amino acids other than X in XRGKXXXXXX or SEQ ID NO: 866 are important for binding to IgE antibodies from allergic patients. Thus, in another preferred mode, amino acids 71-80 of SEQ ID NO: 495 in the antigen in spot 38α may have the amino acid sequence of XRGKXXXXXX or SEQ ID NO: 866.

In the epitope having the sequence of SEQ ID NO: 869, sites of amino acids other than X in RXKXXXXXXXXXXXE are important for binding to IgE antibodies from allergic patients. Thus, in another preferred mode, amino acids 521-535 of SEQ ID NO: 495 in the antigen in spot 38α may have the amino acid sequence of RXKXXXXXXXXXXXE.

In the epitope having the sequence of SEQ ID NO: 871, sites of amino acids other than X in XXXXXXXRRXXXXXR are important for binding to IgE antibodies from allergic patients. Thus, in another preferred mode, amino acids 1-15 of SEQ ID NO: 495 in the antigen in spot 38α may have the amino acid sequence of XXXXXXXRRXXXXXR.

In the epitope having the sequence of SEQ ID NO: 874, sites of amino acids other than X in SEQ ID NO: 872 or 873 are important for binding to IgE antibodies from allergic patients. Thus, in another preferred mode, amino acids 31-45 of SEQ ID NO: 495 in the antigen in spot 38α may have the amino acid sequence of SEQ ID NO: 872 or 873.

In the epitope having the sequence of SEQ ID NO: 877, sites of amino acids other than X in SEQ ID NO: 875 or 876 are important for binding to IgE antibodies from allergic patients. Thus, in another preferred mode, amino acids 221-235 of SEQ ID NO: 495 in the antigen in spot 38α may have the amino acid sequence of SEQ ID NO: 875 or 876.

In the epitope having the sequence of SEQ ID NO: 880, sites of amino acids other than X in SEQ ID NO: 878 or 879 are important for binding to IgE antibodies from allergic patients. Thus, in another preferred mode, amino acids 311-325 of SEQ ID NO: 495 in the antigen in spot 38α may have the amino acid sequence of SEQ ID NO: 878 or 879.

In the epitope having the sequence of SEQ ID NO: 882, sites of amino acids other than X in SEQ ID NO: 881 are important for binding to IgE antibodies from allergic patients. Thus, in another preferred mode, amino acids 461-470 of SEQ ID NO: 495 in the antigen in spot 38α may have the amino acid sequence of SEQ ID NO: 881.

In the epitope having the sequence of SEQ ID NO: 884, sites of amino acids other than X in SEQ ID NO: 883 are important for binding to IgE antibodies from allergic patients. Thus, in another preferred mode, amino acids 481-490 of SEQ ID NO: 495 in the antigen in spot 38 may have the amino acid sequence of SEQ ID NO: 883.

In the epitope having the sequence of SEQ ID NO: 887, sites of amino acids other than X in SEQ ID NO: 885 or 886 are important for binding to IgE antibodies from allergic patients. Thus, in another preferred mode, amino acids 821-835 of SEQ ID NO: 495 in the antigen in spot 38α may have the amino acid sequence of SEQ ID NO: 885 or 886.

In the epitope having the sequence of SEQ ID NO: 889, sites of amino acids other than X in SEQ ID NO: 888 are important for binding to IgE antibodies from allergic patients. Thus, in another preferred mode, amino acids 861-870 of SEQ ID NO: 495 in the antigen in spot 38α may have the amino acid sequence of SEQ ID NO: 888.

In the epitope having the sequence of SEQ ID NO: 892, sites of amino acids other than X in SEQ ID NO: 890 or 891 are important for binding to IgE antibodies from allergic patients. Thus, in another preferred mode, amino acids 1031-1040 of SEQ ID NO: 495 in the antigen in spot 38α may have the amino acid sequence of SEQ ID NO: 890 or 891.

Preferably, a protein that is an antigen in spot 38α has activity equivalent to that of the protein having the amino acid sequence of SEQ ID NO: 495 (seed biotin-containing protein SBP65-like isoform X1) or causative of an allergy to soybean.

### (39α) Antigen in Spot 39α

As the result of sequence identification of the antigen in spot 39α by mass spectroscopy, the amino acid sequence of SEQ ID NO: 516 was detected.

Also, the mass spectroscopic data obtained for spot 39α on a mass spectrometer was analyzed by comparing the data against the NCBI protein data, and as a result, ATP synthase subunit O, mitochondrial-like (amino acid sequence: SEQ ID NO: 515, encoding nucleotide sequence: SEQ ID NO: 514) was identified as a protein comprising SEQ ID NO: 516.

Accordingly, the antigens in spot 39α in the present invention can be any protein selected from the group consisting of the proteins as defined below in (39αA-a) to (39αA-c) and (39β):
(39αA-a) a protein comprising an amino acid sequence with deletion, substitution, insertion or addition of one or several amino acids in SEQ ID NO: 515;
(39αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 515;
(39αA-c) a protein comprising an amino acid sequence encoded by a nucleotide sequence with deletion, substitution, insertion or addition of one or several nucleotides in SEQ ID NO: 514;
(39αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 514;
(39αA-e) a protein comprising an amino acid sequence encoded by a nucleic acid that hybridizes under stringent conditions with a nucleic acid having a nucleotide sequence complementary to the nucleotide sequence of SEQ ID NO: 514;
(39αB) a protein comprising an amino acid sequence of SEQ ID NO: 516. As referred to above, the amino acid sequence(s) of SEQ ID NO: 516 may be an amino acid sequence(s) derived therefrom by deletion, substitution, insertion or addition of one or several amino acids.

The proteins selected from the group consisting of (39αA-a) to (39αA-c) and (39β) as defined above can be proteins that are found in a protein spot with a molecular weight of around 5 to 60 kDa, preferably around 10 to 50 kDa and an isoelectric point of 5.5 to 10.5, preferably 6.0 to 10.0 on gels used in the two-dimensional electrophoresis performed under the conditions described above in the subsection titled “Identification of antigens”.

An epitope of the antigen in spot 39α resides in amino acids 71-85 (SEQ ID NO: 895) of SEQ ID NO: 515. The antigen in spot 39α may retain the sequence in SEQ ID NO: 515 as to amino acids at positions corresponding to the region of this epitope.

From the viewpoint of improving sensitivity (a degree to which a patient can be diagnosed as being positive) or specificity (a degree to which a healthy subject is not diagnosed as being positive), the antigen in spot 39α may be the following variant.

In the epitope having the sequence of SEQ ID NO: 895, sites of amino acids other than X in SEQ ID NO: 893 or 894 are important for binding to IgE antibodies from allergic patients. Thus, in another preferred mode, amino acids 71-85 of SEQ ID NO: 515 in the antigen in spot 39a may have the amino acid sequence of SEQ ID NO: 893 or 894.

Preferably, a protein that is an antigen in spot 39α has activity equivalent to that of the protein having the amino acid sequence of SEQ ID NO: 515 (ATP synthase subunit O, mitochondrial-like) or causative of an allergy to soybean.

### (40α, 56α) Antigens in Spots 40α, 56α

As the result of sequence identification of the antigens in spots 40α and 56α by mass spectroscopy, the amino acid sequences of SEQ ID NOs: 519 and 520 were detected.

Also, the mass spectroscopic data obtained for spots 40α and 56α on a mass spectrometer was analyzed by comparing the data against the NCBI protein data, and as a result, P24 oleosin isoform A (amino acid sequence: SEQ ID NO: 518, encoding nucleotide sequence: SEQ ID NO: 517) was identified as a protein comprising SEQ ID NO: 519 or 520.

Accordingly, the antigens in spot 40α, 56α in the present invention can be any protein selected from the group consisting of the proteins as defined below in (40, 56αA-a) to (40, 56αA-c) and (40, 56αB):
(40, 56αA-a) a protein comprising an amino acid sequence with deletion, substitution, insertion or addition of one or several amino acids in SEQ ID NO: 518;
(40, 56αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 518;
(40, 56αA-c) a protein comprising an amino acid sequence encoded by a nucleotide sequence with deletion, substitution, insertion or addition of one or several nucleotides in SEQ ID NO: 517;
(40, 56αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 517;
(40, 56αA-c) a protein comprising an amino acid sequence encoded by a nucleic acid that hybridizes under stringent conditions with a nucleic acid having a nucleotide sequence complementary to the nucleotide sequence of SEQ ID NO: 517;
(40, 56αB) a protein comprising at least one amino acid sequence selected from the group consisting of SEQ ID NOs: 519 and 520, preferably a protein comprising both the amino acid sequences. As referred to above, the amino acid sequence of any of SEQ ID NOs: 519 and 520 may be an amino acid sequence derived therefrom by deletion, substitution, insertion or addition of one or several amino acids.

The proteins selected from the group consisting of (40, 56αA-a) to (40, 56αA-c) and (40, 56αB) as defined above can be proteins that are found in a protein spot with a molecular weight of around 5 to 60 kDa, preferably around 10 to 50 kDa and an isoelectric point of 5.5 to 10.5, preferably 6.0 to 10.0 on gels used in the two-dimensional electrophoresis performed under the conditions described above in the subsection titled “Identification of antigens”.

Epitopes of the antigens in spots 40α and 56α reside in amino acids 35-39 (SEQ ID NO: 898), amino acids 191-205 (SEQ ID NO: 899) and amino acids 214-222 (SEQ ID NO: 902) of SEQ ID NO: 518. The antigens in spots 40α and 56α may retain the sequence in SEQ ID NO: 518 as to amino acids at positions corresponding to the region of these epitopes.

From the viewpoint of improving sensitivity (a degree to which a patient can be diagnosed as being positive) or specificity (a degree to which a healthy subject is not diagnosed as being positive), the antigen in spot 40α, 56a may be the following variant.

In the epitope having the sequence of SEQ ID NO: 898, sites of amino acids other than X in SEQ ID NO: 896 or 897 are important for binding to IgE antibodies from allergic patients. Thus, in another preferred mode, amino acids 35-39 of SEQ ID NO: 518 in the antigen in spot 40α, 56a may have the amino acid sequence of SEQ ID NO: 896 or 897.

In the epitope having the sequence of SEQ ID NO: 902, sites of amino acids other than X in XXXXXVTAX or SEQ ID NO: 901 are important for binding to IgE antibodies from allergic patients. Thus, in another preferred mode, amino acids 214-2229 of SEQ ID NO: 518 in the antigen in spot 40α, 56α may have the amino acid sequence of XXXXXVTAX or SEQ ID NO: 901.

Preferably, a protein that is an antigen in spot 40α or 56α has activity equivalent to that of the protein having the amino acid sequence of SEQ ID NO: 518 (P24 oleosin isoform A) or causative of an allergy to soybean.

### (41α) Antigen in Spot 41α

As the result of sequence identification of the antigen in spot 41α by mass spectroscopy, the amino acid sequence of SEQ ID NO: 523 was detected.

Also, the mass spectroscopic data obtained for spot 41a on a mass spectrometer was analyzed by comparing the data against the NCBI protein data, and as a result, uncharacterized protein At3g49720-like isoform X2 (amino acid sequence: SEQ ID NO: 522, encoding nucleotide sequence: SEQ ID NO: 521) was identified as a protein comprising SEQ ID NO: 523.

Accordingly, the antigens in spot 41a in the present invention can be any protein selected from the group consisting of the proteins as defined below in (41αA-a) to (41αA-c) and (41αB):
(4l αA-a) a protein comprising an amino acid sequence with deletion, substitution, insertion or addition of one or several amino acids in SEQ ID NO: 522;
(41αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 522;
(41αA-c) a protein comprising an amino acid sequence encoded by a nucleotide sequence with deletion, substitution, insertion or addition of one or several nucleotides in SEQ ID NO: 521;
(41αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 521;
(41αA-c) a protein comprising an amino acid sequence encoded by a nucleic acid that hybridizes under stringent conditions with a nucleic acid having a nucleotide sequence complementary to the nucleotide sequence of SEQ ID NO: 521;
(41αB) a protein comprising an amino acid sequence of SEQ ID NO: 523. As referred to above, the amino acid sequence of SEQ ID NO: 523 may be an amino acid sequence derived therefrom by deletion, substitution, insertion or addition of one or several amino acids.

The proteins selected from the group consisting of (41αA-a) to (41αA-c) and (41αB) as defined above can be proteins that are found in a protein spot with a molecular weight of around 5 to 60 kDa, preferably around 10 to 50 kDa and an isoelectric point of 5.5 to 10.5, preferably 6.0 to 10.0 on gels used in the two-dimensional electrophoresis performed under the conditions described above in the subsection titled “Identification of antigens”.

An epitope of the antigen in spot 41α resides in amino acids 111-125 (SEQ ID NO: 903) of SEQ ID NO: 522. The antigen in spot 41α may retain the sequence in SEQ ID NO: 522 as to amino acids at positions corresponding to the region of this epitope.

Preferably, a protein that is an antigen in spot 41a has activity equivalent to that of the protein having the amino acid sequence of SEQ ID NO: 515 (ATP synthase subunit O, mitochondrial-like) or causative of an allergy to soybean.

### (42α, 43α) Antigen in Spots 42α, 43α

As the result of sequence identification of the antigens in spots 42α and 43α by mass spectroscopy, the amino acid sequences of SEQ ID NOs: 526-539 were detected.

Also, the mass spectroscopic data obtained for spots 42α and 43α on a mass spectrometer was analyzed by comparing the data against the NCBI protein data, and as a result, superoxide dismutase [Mn], mitochondrial (amino acid sequence: SEQ ID NO: 525, encoding nucleotide sequence: SEQ ID NO: 524) was identified as a protein comprising any of SEQ ID NOs: 526 to 531 and hypothetical protein GLYMA_04G221300 (amino acid sequence: SEQ ID NO: 533, encoding nucleotide sequence: SEQ ID NO: 532) was identified as a protein comprising any of SEQ ID NOs: 534 to 539.

Accordingly, the antigen in spot 42α, 43α in the present invention can be any of proteins selected from the group consisting of the proteins as defined below in (42, 43αA1-a) to (42, 43αA1-c), (42, 43αB1), (42, 43αA2-a) to (42, 43A2-c), and (42, 43αB2):
(42, 43αA1-a) a protein comprising an amino acid sequence with deletion, substitution, insertion or addition of one or several amino acids in SEQ ID NO: 525;
(42, 43αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 525;
(42, 43αA1-c) a protein comprising an amino acid sequence encoded by a nucleotide sequence with deletion, substitution, insertion or addition of one or several nucleotides in SEQ ID NO: 524;
(42, 43αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 524;
(42, 43αA1-e) a protein comprising an amino acid sequence encoded by a nucleic acid that hybridizes under stringent conditions with a nucleic acid having a nucleotide sequence complementary to the nucleotide sequence of SEQ ID NO: 524;
(42, 43β1) a protein comprising at least one amino acid sequence selected from the group consisting of SEQ ID NOs: 526-531, preferably a protein comprising at least 2, 3, 4, or 5 or all sequences of the amino acid sequences. As referred to above, the amino acid sequence of any of SEQ ID NOs: 526 to 531 may be an amino acid sequence derived therefrom by deletion, substitution, insertion or addition of one or several amino acids;
(42, 43αA2-a) a protein comprising an amino acid sequence with deletion, substitution, insertion or addition of one or several amino acids in SEQ ID NO: 533;
(42, 43αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 533;
(42, 43αA2-c) a protein comprising an amino acid sequence encoded by a nucleotide sequence with deletion, substitution, insertion or addition of one or several nucleotides in SEQ ID NO: 532;
(42, 43αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 532;
(42, 43αA2-c) a protein comprising an amino acid sequence encoded by a nucleic acid that hybridizes under stringent conditions with a nucleic acid having a nucleotide sequence complementary to the nucleotide sequence of SEQ ID NO: 532;
(42, 43αB2) a protein comprising at least one amino acid sequence selected from the group consisting of SEQ ID NOs: 534-539, preferably a protein comprising at least 2, 3, 4, or 5 or all sequences of the amino acid sequences. As referred to above, the amino acid sequence of any of SEQ ID NOs: 534 to 539 may be an amino acid sequence derived therefrom by deletion, substitution, insertion or addition of one or several amino acids.

The protein selected from the group consisting of the proteins as defined above in (42, 43αA1-a) to (42, 43αA1-c), (42, 43αB1), (42, 43αA2-a) to (42, 43αA2-c), and (42, 43αB2) can be proteins that are found in a protein spot with a molecular weight of around 5 to 60 kDa, preferably around 10 to 50 kDa, and an isoelectric point of 5.5 to 10.5, preferably 6.0 to 10.0 on gels used in the two-dimensional electrophoresis performed under the conditions described above in the subsection titled “Identification of antigens”.

Epitopes of the aforementioned antigens (42, 43αA1-a) to (42, 43αA1-c) and (42, 43αB1) reside in amino acids 227-241 (SEQ ID NO: 906) of SEQ ID NO: 525. When the antigen in spot 42α, 43a contains a variation in the amino acid sequence of SEQ ID NO: 525, this antigen may retain the sequence in SEQ ID NO: 525 as to amino acids at positions corresponding to the region of at least one of these epitopes.

From the viewpoint of improving sensitivity (a degree to which a patient can be diagnosed as being positive) or specificity (a degree to which a healthy subject is not diagnosed as being positive), the antigen in spot 42α, 43 may be the following variant.

In the epitope having the sequence of SEQ ID NO: 906, sites of amino acids other than X in SEQ ID NO: 904 or 905 are important for binding to IgE antibodies from allergic patients. Thus, in another preferred mode, when the antigen in spot 42α, 43a contains a variation in the amino acid sequence of SEQ ID NO: 525, amino acids 227-241 of SEQ ID NO: 525 in this antigen may have the amino acid sequence of SEQ ID NO: 904 or 905.

Preferably, a protein that is an antigen in spot 42α or 43α has activity equivalent to that of the protein having the amino acid sequence of SEQ ID NO: 525 (superoxide dismutase [Mn], mitochondrial) or the protein having the amino acid sequence of SEQ ID NO: 533 (hypothetical protein GLYMA_04G221300) or causative of an allergy to soybean.

### (45α) Antigen in Spot 45α

As the result of sequence identification of the antigen in spot 45α by mass spectroscopy, the amino acid sequences of SEQ ID NOs: 542-551 were detected.

Also, the mass spectroscopic data obtained for spot 45α on a mass spectrometer was analyzed by comparing the data against the NCBI protein data, and as a result, uncharacterized protein LOC100499771 (amino acid sequence: SEQ ID NO: 541, encoding nucleotide sequence: SEQ ID NO: 540) was identified as a protein comprising any of SEQ ID NOs: 542 to 545 and hypothetical protein GLYMA_09G192800 (amino acid sequence: SEQ ID NO: 547, encoding nucleotide sequence: SEQ ID NO: 546) was identified as a protein comprising any of SEQ ID NOs: 548 to 551.

Accordingly, the antigen in spot 45α in the present invention can be any of proteins selected from the group consisting of the proteins as defined below in (45αA1-a) to (45αA1-c), (45αB1), (45αA2-a) to (45αA2c), and (45αB2):
(45αA1-a) a protein comprising an amino acid sequence with deletion, substitution, insertion or addition of one or several amino acids in SEQ ID NO: 541;
(45αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 541;
(45αA1-c) a protein comprising an amino acid sequence encoded by a nucleotide sequence with deletion, substitution, insertion or addition of one or several nucleotides in SEQ ID NO: 540;
(45αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 540;
(45αA1-c) a protein comprising an amino acid sequence encoded by a nucleic acid that hybridizes under stringent conditions with a nucleic acid having a nucleotide sequence complementary to the nucleotide sequence of SEQ ID NO: 540;
(45αB1) a protein comprising at least one amino acid sequence selected from the group consisting of SEQ ID NOs: 542-545, preferably a protein comprising at least 2, 3, or all sequences of the amino acid sequences. As referred to above, the amino acid sequence of any of SEQ ID NOs: 542 to 545 may be an amino acid sequence derived therefrom by deletion, substitution, insertion or addition of one or several amino acids;
(45αA2-a) a protein comprising an amino acid sequence with deletion, substitution, insertion or addition of one or several amino acids in SEQ ID NO: 547;
(45αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 547;
(45αA2-c) a protein comprising an amino acid sequence encoded by a nucleotide sequence with deletion, substitution, insertion or addition of one or several nucleotides in SEQ ID NO: 546;
(45αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 546;
(45αA2-e) a protein comprising an amino acid sequence encoded by a nucleic acid that hybridizes under stringent conditions with a nucleic acid having a nucleotide sequence complementary to the nucleotide sequence of SEQ ID NO: 546;
(45β2) a protein comprising at least one amino acid sequence selected from the group consisting of SEQ ID NOs: 548-551, preferably a protein comprising at least 2, 3, or all sequences of the amino acid sequences. As referred to above, the amino acid sequence of any of SEQ ID NOs: 548 to 551 may be an amino acid sequence derived therefrom by deletion, substitution, insertion or addition of one or several amino acids.

The protein selected from the group consisting of the proteins as defined above in (45αA1-a) to (45αA1-c), (45αB1), (45αA2-a) to (45αA2-c), and (45αB2) can be proteins that are found in a protein spot with a molecular weight of around 2 to 50 kDa, preferably around 5 to 40 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (45αA1-a) to (45αA1-c) and (45αB1) reside in amino acids 11-25 (SEQ ID NO: 910), amino acids 51-65 (SEQ ID NO: 911), amino acids 111-125 (SEQ ID NO: 912) of SEQ ID NO: 541. When the antigen in spot 45a contains a variation in the amino acid sequence of SEQ ID NO: 541, amino acids at positions corresponding to the region of at least one of these epitopes may retain the sequence in SEQ ID NO: 541.

Preferably, a protein that is an antigen in spot 45α has activity equivalent to that of the protein having the amino acid sequence of SEQ ID NO: 541 (uncharacterized protein LOC100499771) or the protein having the amino acid sequence of SEQ ID NO: 547 (hypothetical protein GLYMA_09G192800) or causative of an allergy to soybean.

### (46α) Antigen in Spot 46α

As the result of sequence identification of the antigen in spot 46α by mass spectroscopy, the amino acid sequence of SEQ ID NOs: 554 was detected.

Also, the mass spectroscopic data obtained for spot 46α on a mass spectrometer was analyzed by comparing the data against the NCBI protein data, and as a result, seed maturation protein PM22 (amino acid sequence: SEQ ID NO: 553, encoding nucleotide sequence: SEQ ID NO: 552) was identified as a protein comprising SEQ ID NO: 554.

Accordingly, the antigens in spot 46α in the present invention can be any protein selected from the group consisting of the proteins as defined below in (46αA-a) to (46αA-c) and (46αB):
(46αA-a) a protein comprising an amino acid sequence with deletion, substitution, insertion or addition of one or several amino acids in SEQ ID NO: 553;
(46αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 553;
(46αA-c) a protein comprising an amino acid sequence encoded by a nucleotide sequence with deletion, substitution, insertion or addition of one or several nucleotides in SEQ ID NO: 552;
(46αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 552;
(46αA-c) a protein comprising an amino acid sequence encoded by a nucleic acid that hybridizes under stringent conditions with a nucleic acid having a nucleotide sequence complementary to the nucleotide sequence of SEQ ID NO: 552;
(46αB) a protein comprising an amino acid sequence of SEQ ID NO: 554. As referred to.: above, the amino acid sequence(s) of SEQ ID NO: 554 may be an amino acid sequence(s) derived therefrom by deletion, substitution, insertion or addition of one or several amino acids.

The proteins selected from the group consisting of (46αA-a) to (46αA-c) and (46αB) as defined above can be proteins that are found in a protein spot with a molecular weight of around 2 to 50 kDa, preferably around 5 to 40 kDa and an isoelectric point of 3.5 to 7.5, preferably 4.0 to 7.0 on gels used in the two-dimensional electrophoresis performed under the conditions described above in the subsection titled “Identification of antigens”.

An epitope of the antigen in spot 46α resides in amino acids 112-120 (SEQ ID NO: 915) of SEQ ID NO: 553. The antigen in spot 46α may retain the sequence in SEQ ID NO: 553 as to amino acids at positions corresponding to the region of this epitope.

From the viewpoint of improving sensitivity (a degree to which a patient can be diagnosed as being positive) or specificity (a degree to which a healthy subject is not diagnosed as being positive), the antigen in spot 46α may be the following variant.

In the epitope having the sequence of SEQ ID NO: 915, sites of amino acids other than X in XDXDXXXDX or SEQ ID NO: 914 are important for binding to IgE antibodies from allergic patients. Thus, in another preferred mode, amino acids 112-120 of SEQ ID NO: 553 in the antigen in spot 46α may have the amino acid sequence of XDXDXXXDX or SEQ ID NO: 914.

Preferably, a protein that is an antigen in spot 46α has activity equivalent to that of the protein having the amino acid sequence of SEQ ID NO: 553 (seed maturation protein PM22) or causative of an allergy to soybean.

### (47α) Antigen in Spot 47α

As the result of sequence identification of the antigen in spot 47α by mass spectroscopy, the amino acid sequences of SEQ ID NOs: 557-563 were detected.

Also, the mass spectroscopic data obtained for spot 47a on a mass spectrometer was analyzed by comparing the data against the NCBI protein data, and as a result, eukaryotic translation initiation factor 5A-2 (amino acid sequence: SEQ ID NO: 556, encoding nucleotide sequence: SEQ ID NO: 555) was identified as a protein comprising SEQ ID NO: 557 or 558 and uncharacterized protein LOC100305510 (amino acid sequence: SEQ ID NO: 560, encoding nucleotide sequence: SEQ ID NO: 559) was identified as a protein comprising any of SEQ ID NOs: 561 to 563.

Accordingly, the antigen in spot 47α in the present invention can be any of proteins selected from the group consisting of the proteins as defined below in (47αA1-a) to (47αA1-e), (47αB1), (47αA2-a) to (47αA2-c), and (47αB2):
(47αA1-a) a protein comprising an amino acid sequence with deletion, substitution, insertion or addition of one or several amino acids in SEQ ID NO: 556;
(47αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 556;
(47αA1-c) a protein comprising an amino acid sequence encoded by a nucleotide sequence with deletion, substitution, insertion or addition of one or several nucleotides in SEQ ID NO: 555;
(47αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 555;
(47αA1-e) a protein comprising an amino acid sequence encoded by a nucleic acid that hybridizes under stringent conditions with a nucleic acid having a nucleotide sequence complementary to the nucleotide sequence of SEQ ID NO: 555;
(47αB1) a protein comprising at least one amino acid sequence selected from the group consisting of SEQ ID NOs: 557 and 558, preferably a protein comprising both the amino acid sequences. As referred to above, the amino acid sequence of any of SEQ ID NOs: 557, 558 may be an amino acid sequence derived therefrom by deletion, substitution, insertion or addition of one or several amino acids;
(47αA2-a) a protein comprising an amino acid sequence with deletion, substitution, insertion or addition of one or several amino acids in SEQ ID NO: 560;
(47αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 560;
(47αA2-c) a protein comprising an amino acid sequence encoded by a nucleotide sequence with deletion, substitution, insertion or addition of one or several nucleotides in SEQ ID NO: 559;
(47αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 559;
(47αA2-c) a protein comprising an amino acid sequence encoded by a nucleic acid that hybridizes under stringent conditions with a nucleic acid having a nucleotide sequence complementary to the nucleotide sequence of SEQ ID NO: 559;
(47αB2) a protein comprising at least one amino acid sequence selected from the group consisting of SEQ ID NOs: 561-563, preferably a protein comprising at least 2, or all sequences of the amino acid sequences. As referred to above, the amino acid sequence of any of SEQ ID NOs: 548 to 551 may be an amino acid sequence derived therefrom by deletion, substitution, insertion or addition of one or several amino acids.

The protein selected from the group consisting of the proteins as defined above in (47αA1-a) to (47αA1-c), (47αB1), (47αA2-a) to (47αA2-c), and (47αB2) can be proteins that are found in a protein spot with a molecular weight of around 2 to 50 kDa, preferably around 5 to 40 kDa, and an isoelectric point of 3.5 to 7.5, preferably 4.0 to 7.0 on gels used in the two-dimensional electrophoresis performed under the conditions described above in the subsection titled “Identification of antigens”.

Epitopes of the aforementioned antigens (47αA1-a) to (47αA1-e) and (47αB1) reside in amino acids 11-25 (SEQ ID NO: 916), amino acids 81-95 (SEQ ID NO: 917), amino acids 131-145 (SEQ ID NO: 918) of SEQ ID NO: 556. When the antigen in spot 47a contains a variation in the amino acid sequence of SEQ ID NO: 556, amino acids at positions corresponding to the region of at least one of these epitopes may retain the sequence in SEQ ID NO: 556.

Preferably, a protein that is an antigen in spot 47α has activity equivalent to that of the protein having the amino acid sequence of SEQ ID NO: 556 (eukaryotic translation initiation factor 5A-2) or the protein having the amino acid sequence of SEQ ID NO: 560 (uncharacterized protein LOC100305510) or causative of an allergy to soybean.

### (48α) Antigen in Spot 48α

As the result of sequence identification of the antigen in spot 48 by mass spectroscopy, the amino acid sequences of SEQ ID NOs: 566-570 were detected.

Also, the mass spectroscopic data obtained for spot 48α on a mass spectrometer was analyzed by comparing the data against the NCBI protein data, and as a result, uncharacterized protein LOC100306570 (amino acid sequence: SEQ ID NO: 565, encoding nucleotide sequence: SEQ ID NO: 564) was identified as a protein comprising any of SEQ ID NOs: 566 to 570.

Accordingly, the antigens in spot 48α in the present invention can be any protein selected from the group consisting of the proteins as defined below in (48αA-a) to (48αA-c) and (48«B):
(48αA-a) a protein comprising an amino acid sequence with deletion, substitution, insertion or addition of one or several amino acids in SEQ ID NO: 565;
(48αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 565;
(48αA-c) a protein comprising an amino acid sequence encoded by a nucleotide sequence with deletion, substitution, insertion or addition of one or several nucleotides in SEQ ID NO: 564;
(48αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 564;
(48αA-c) a protein comprising an amino acid sequence encoded by a nucleic acid that hybridizes under stringent conditions with a nucleic acid having a nucleotide sequence complementary to the nucleotide sequence of SEQ ID NO: 564;
(48αB) a protein comprising at least one amino acid sequence selected from the group consisting of SEQ ID NOs: 566-570, preferably a protein comprising at least 2, 3 or 4 or all sequences of the amino acid sequences. As referred to above, the amino acid sequence of any of SEQ ID NOs: 566-570 may be an amino acid sequence derived therefrom by deletion, substitution, insertion or addition of one or several amino acids.

The proteins selected from the group consisting of (48αA-a) to (48αA-c) and (48&B) as defined above can be proteins that are found in a protein spot with a molecular weight of around 2 to 50 kDa, preferably around 5 to 40 kDa and an isoelectric point of 3.5 to 8.5, preferably 4.0 to 8.0 on gels used in the two-dimensional electrophoresis performed under the conditions described above in the subsection titled “Identification of antigens”.

Epitopes of the antigen in spot 48α reside in amino acids 94-102 (SEQ ID NO: 921) and amino acids 131-145 (SEQ ID NO: 922) of SEQ ID NO: 565. The antigen in spot 48α may retain the sequence in SEQ ID NO: 565 as to amino acids at positions corresponding to the region of these epitopes.

From the viewpoint of improving sensitivity (a degree to which a patient can be diagnosed as being positive) or specificity (a degree to which a healthy subject is not diagnosed as being positive), the antigen in spot 48α may be the following variant.

In the epitope having the sequence of SEQ ID NO: 921, sites of amino acids other than X in XXXXXXFDK or SEQ ID NO: 918 are important for binding to IgE antibodies from allergic patients. Thus, in another preferred mode, amino acids 94-102 of SEQ ID NO: 565 in the antigen in spot 48α may have the amino acid sequence of XXXXXXFDK or SEQ ID NO: 918.

Preferably, a protein that is an antigen in spot 48α has activity equivalent to that of the protein having the amino acid sequence of SEQ ID NO: 565 (uncharacterized protein LOC100306570) or causative of an allergy to soybean.

### (50α) Antigen in Spot 50α

As the result of sequence identification of the antigen in spot 50α by mass spectroscopy, the amino acid sequences of SEQ ID NOs: 573-576 were detected.

Also, the mass spectroscopic data obtained for spot 50α on a mass spectrometer was analyzed by comparing the data against the NCBI protein data, and as a result, uncharacterized protein LOC100813859 (amino acid sequence: SEQ ID NO: 572, encoding nucleotide sequence: SEQ ID NO: 571) was identified as a protein comprising any of SEQ ID NOs: 573 to 576.

Accordingly, the antigens in spot 50α in the present invention can be any protein selected from the group consisting of the proteins as defined below in (50αA-a) to (50αA-e) and (50«B):
(50αA-a) a protein comprising an amino acid sequence with deletion, substitution, insertion or addition of one or several amino acids in SEQ ID NO: 572;
(50αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 572;
(50αA-c) a protein comprising an amino acid sequence encoded by a nucleotide sequence with deletion, substitution, insertion or addition of one or several nucleotides in SEQ ID NO: 571;
(50αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 571;
(50αA-e) a protein comprising an amino acid sequence encoded by a nucleic acid that hybridizes under stringent conditions with a nucleic acid having a nucleotide sequence complementary to the nucleotide sequence of SEQ ID NO: 571;
(50αB) a protein comprising at least one amino acid sequence selected from the group consisting of SEQ ID NOs: 573-576, preferably a protein comprising at least 2 or 3 or all sequences of the amino acid sequences. As referred to above, the amino acid sequence of any of SEQ ID NOs: 573-576 may be an amino acid sequence derived therefrom by deletion, substitution, insertion or addition of one or several amino acids.

The proteins selected from the group consisting of (50αA-a) to (50αA-c) and (50αB) as defined above can be proteins that are found in a protein spot with a molecular weight of around 2 to 50 kDa, preferably around 5 to 40 kDa and an isoelectric point of 2.5 to 6.5, preferably 3.0 to 6.0 on gels used in the two-dimensional electrophoresis performed under the conditions described above in the subsection titled “Identification of antigens”.

Epitopes of the antigen in spot 50α reside in amino acids 91-105 (SEQ ID NO: 924) and amino acids 62-70 (SEQ ID NO: 927) of SEQ ID NO: 572. The antigen in spot 50α may retain the sequence in SEQ ID NO: 572 as to amino acids at positions corresponding to the region of these epitopes.

From the viewpoint of improving sensitivity (a degree to which a patient can be diagnosed as being positive) or specificity (a degree to which a healthy subject is not diagnosed as being positive), the antigen in spot 50α may be the following variant.

In the epitope having the sequence of SEQ ID NO: 927, sites of amino acids other than X in XXXXKQXXX or XXDXKQXXX are important for binding to IgE antibodies from allergic patients. Thus, in another preferred mode, amino acids 62-70 of SEQ ID NO: 572 in the antigen in spot 50α may have the amino acid sequence of XXXXKQXXX or XXDXKQXXX.

Preferably, a protein that is an antigen in spot 50α has activity equivalent to that of the protein having the amino acid sequence of SEQ ID NO: 572 (uncharacterized protein LOC100813859) or causative of an allergy to soybean.

### (51α) Antigen in Spot 51α

As the result of sequence identification of the antigen in spot 51 by mass spectroscopy, the amino acid sequences of SEQ ID NOs: 579-582 were detected.

Also, the mass spectroscopic data obtained for spot 51a on a mass spectrometer was analyzed by comparing the data against the NCBI protein data, and as a result, 14-3-3-like protein (amino acid sequence: SEQ ID NO: 578, encoding nucleotide sequence: SEQ ID NO: 577) was identified as a protein comprising any of SEQ ID NOs: 579 to 582.

Accordingly, the antigens in spot 51α in the present invention can be any protein selected from the group consisting of the proteins as defined below in (51αA-a) to (51αA-c) and (51αB):
(51αA-a) a protein comprising an amino acid sequence with deletion, substitution, insertion or addition of one or several amino acids in SEQ ID NO: 578;
(51αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 578;
(51αA-c) a protein comprising an amino acid sequence encoded by a nucleotide sequence with deletion, substitution, insertion or addition of one or several nucleotides in SEQ ID NO: 577;
(51αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 577;
(51αA-c) a protein comprising an amino acid sequence encoded by a nucleic acid that hybridizes under stringent conditions with a nucleic acid having a nucleotide sequence complementary to the nucleotide sequence of SEQ ID NO: 577;
(51αB) a protein comprising at least one amino acid sequence selected from the group consisting of SEQ ID NOs: 579-582, preferably a protein comprising at least 2 or 3 or all sequences of the amino acid sequences. As referred to above, the amino acid sequence of any of SEQ ID NOs: 579-582 may be an amino acid sequence derived therefrom by deletion, substitution, insertion or addition of one or several amino acids.

The proteins selected from the group consisting of (51αA-a) to (51αA-c) and (51αB) as defined above can be proteins that are found in a protein spot with a molecular weight of around 5 to 60 kDa, preferably around 10 to 50 kDa and an isoelectric point of 2.5 to 6.5, preferably 3.0 to 6.0 on gels used in the two-dimensional electrophoresis performed under the conditions described above in the subsection titled “Identification of antigens”.

Epitopes of the antigen in spot 51α reside in amino acids 71-85 (SEQ ID NO: 928), amino acids 141-155 (SEQ ID NO: 929) and amino acids 211-225 (SEQ ID NO: 930) of SEQ ID NO: 578. The antigen in spot 51α may retain the sequence in SEQ ID NO: 578 as to amino acids at positions corresponding to the region of these epitopes.

Preferably, a protein that is an antigen in spot 51α has activity equivalent to that of the protein having the amino acid sequence of SEQ ID NO: 578 (14-3-3-like protein) or causative of an allergy to soybean.

### (52α) Antigen in Spot 52α

As the result of sequence identification of the antigen in spot 52α by mass spectroscopy, the amino acid sequences of SEQ ID NOs: 585-588 were detected.

Also, the mass spectroscopic data obtained for spot 52α on a mass spectrometer was analyzed by comparing the data against the NCBI protein data, and as a result, probable fructokinase-4 (amino acid sequence: SEQ ID NO: 584, encoding nucleotide sequence: SEQ ID NO: 583) was identified as a protein comprising any of SEQ ID NOs: 585 to 588.

Accordingly, the antigens in spot 52α in the present invention can be any protein selected from the group consisting of the proteins as defined below in (52αA-a) to (52αA-c) and (52«B):
(52αA-a) a protein comprising an amino acid sequence with deletion, substitution, insertion or addition of one or several amino acids in SEQ ID NO: 584;
(52αA-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 584;
(52αA-c) a protein comprising an amino acid sequence encoded by a nucleotide sequence with deletion, substitution, insertion or addition of one or several nucleotides in SEQ ID NO: 583;
(52αA-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 583;
(52αA-e) a protein comprising an amino acid sequence encoded by a nucleic acid that hybridizes under stringent conditions with a nucleic acid having a nucleotide sequence complementary to the nucleotide sequence of SEQ ID NO: 583;
(52αB) a protein comprising at least one amino acid sequence selected from the group consisting of SEQ ID NOs: 585-588, preferably a protein comprising at least 2 or 3 or all sequences of the amino acid sequences. As referred to above, the amino acid sequence of any of SEQ ID NOs: 585-588 may be an amino acid sequence derived therefrom by deletion, substitution, insertion or addition of one or several amino acids.

The proteins selected from the group consisting of (52αA-a) to (52αA-c) and (52αB) as defined above can be proteins that are found in a protein spot with a molecular weight of around 5 to 60 kDa, preferably around 10 to 50 kDa and an isoelectric point of 3.5 to 7.5, preferably 4.0 to 7.0 on gels used in the two-dimensional electrophoresis performed under the conditions described above in the subsection titled “Identification of antigens”.

Epitopes of the antigen in spot 52α reside in amino acids 11-25 (SEQ ID NO: 933), amino acids 71-85 (SEQ ID NO: 934), amino acids 121-135 (SEQ ID NO: 935), amino acids 201-215 (SEQ ID NO: 936), amino acids 211-225 (SEQ ID NO: 937), and amino acids 281-295 (SEQ ID NO: 938) of SEQ ID NO: 584. The antigen in spot 52α may retain the sequence in SEQ ID NO: 584 as to amino acids at positions corresponding to the region of these epitopes.

From the viewpoint of improving sensitivity (a degree to which a patient can be diagnosed as being positive) or specificity (a degree to which a healthy subject is not diagnosed as being positive), the antigen in spot 52α may be the following variant.

In the epitope having the sequence of SEQ ID NO: 933, sites of amino acids other than X in XXXXXXSXXEXLXXX or SEQ ID NO: 932 are important for binding to IgE antibodies from allergic patients. Thus, in another preferred mode, amino acids 11-25 of SEQ ID NO: 584 in the antigen in spot 52α may have the amino acid sequence of XXXXXXSXXEXLXXX or SEQ ID NO: 932.

Preferably, a protein that is an antigen in spot 52α has activity equivalent to that of the protein having the amino acid sequence of SEQ ID NO: 584 (probable fructokinase-4) or causative of an allergy to soybean.

### (53α) Antigen in Spot 53α

As the result of sequence identification of the antigen in spot 53α by mass spectroscopy, the amino acid sequences of SEQ ID NOs: 591-604 were detected.

Also, the mass spectroscopic data obtained for spot 53α on a mass spectrometer was analyzed by comparing the data against the NCBI protein data, and as a result, triosephosphate isomerase isoform X1 (amino acid sequence: SEQ ID NO: 590, encoding nucleotide sequence: SEQ ID NO: 589) was identified as a protein comprising any of SEQ ID NOs: 591 to 597 and triosephosphate isomerase, cytosolic (amino acid sequence: SEQ ID NO: 599, encoding nucleotide sequence: SEQ ID NO: 598) was identified as a protein comprising any of SEQ ID NOs: 600 to 604.

Accordingly, the antigen in spot 53α in the present invention can be any of proteins selected from the group consisting of the proteins as defined below in (53αA1-a) to (53αA1-c), (53αB1), (53αA2-a) to (53αA2-c), and (53αB2):
(53αA1-a) a protein comprising an amino acid sequence with deletion, substitution, insertion or addition of one or several amino acids in SEQ ID NO: 590;
(53αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 590;
(53αA1-c) a protein comprising an amino acid sequence encoded by a nucleotide sequence with deletion, substitution, insertion or addition of one or several nucleotides in SEQ ID NO: 589;
(53αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 589;
(53αA1-c) a protein comprising an amino acid sequence encoded by a nucleic acid that hybridizes under stringent conditions with a nucleic acid having a nucleotide sequence complementary to the nucleotide sequence of SEQ ID NO: 589;
(53αB1) a protein comprising at least one amino acid sequence selected from the group consisting of SEQ ID NOs: 591-597, preferably a protein comprising at least 2, 3, 4, 5, or 6 or all sequences of the amino acid sequences. As referred to above, the amino acid sequence of any of SEQ ID NOs: 591 to 597 may be an amino acid sequence derived therefrom by deletion, substitution, insertion or addition of one or several amino acids;
(53αA2-a) a protein comprising an amino acid sequence with deletion, substitution, insertion or addition of one or several amino acids in SEQ ID NO: 599;
(53αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 599;
(53αA2-c) a protein comprising an amino acid sequence encoded by a nucleotide sequence with deletion, substitution, insertion or addition of one or several nucleotides in SEQ ID NO: 598;
(53αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 598;
(53αA2-e) a protein comprising an amino acid sequence encoded by a nucleic acid that hybridizes under stringent conditions with a nucleic acid having a nucleotide sequence complementary to the nucleotide sequence of SEQ ID NO: 598;
(53αB2) a protein comprising at least one amino acid sequence selected from the group consisting of SEQ ID NOs: 600-604, preferably a protein comprising at least 2, 3, or 4 or all sequences of the amino acid sequences. As referred to above, the amino acid sequence of any of SEQ ID NOs: 600 to 604 may be an amino acid sequence derived therefrom by deletion, substitution, insertion or addition of one or several amino acids.

The protein selected from the group consisting of the proteins as defined above in (53αA1-a) to (53αA1-c), (53αB1), (53αA2-a) to (53αA2-c), and (53αB2) can be proteins that are found in a protein spot with a molecular weight of around 5 to 60 kDa, preferably around 10 to 50 kDa, and an isoelectric point of 3.5 to 8.5, preferably 4.0 to 8.0 on gels used in the two-dimensional electrophoresis performed under the conditions described above in the subsection titled “Identification of antigens”.

Epitopes of the aforementioned antigens (53αA1-a) to (53αA1-e) and (53«B1) reside in amino acids 71-85 (SEQ ID NO: 939) of SEQ ID NO: 590. When the antigen in spot 53α contains a variation in the amino acid sequence of SEQ ID NO: 590, amino acids at positions corresponding to the region of at least one of these epitopes may retain the sequence in SEQ ID NO: 590.

Preferably, a protein that is an antigen in spot 53α has activity equivalent to that of the protein having the amino acid sequence of SEQ ID NO: 590 (triosephosphate isomerase isoform X1) or the protein having the amino acid sequence of SEQ ID NO: 599 (triosephosphate isomerase, cytosolic) or causative of an allergy to soybean.

### (54α) Antigen in Spot 54α

As the result of sequence identification of the antigen in spot 54α by mass spectroscopy, the amino acid sequences of SEQ ID NOs: 607-616 were detected.

Also, the mass spectroscopic data obtained for spot 54α on a mass spectrometer was analyzed by comparing the data against the NCBI protein data, and as a result, superoxide dismutase [Fe], chloroplastic isoform X1 (amino acid sequence: SEQ ID NO: 606, encoding nucleotide sequence: SEQ ID NO: 605) as a protein comprising any of SEQ ID NOs: 607 to 611 and iron-superoxide dismutase (amino acid sequence: SEQ ID NO: 613, encoding nucleotide sequence: SEQ ID NO: 612) was identified as a protein comprising any of SEQ ID NOs: 614 to 616.

Accordingly, the antigen in spot 54α in the present invention can be any of proteins selected from the group consisting of the proteins as defined below in (54αA1-a) to (54αA1-e), (54αB1), (54αA2-a) to (54αA2-c), and (54αB2):
(54αA1-a) a protein comprising an amino acid sequence with deletion, substitution, insertion or addition of one or several amino acids in SEQ ID NO: 606;
(54αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 606;
(54αA1-c) a protein comprising an amino acid sequence encoded by a nucleotide sequence with deletion, substitution, insertion or addition of one or several nucleotides in SEQ ID NO: 605;
(54αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 605;
(54αA1-c) a protein comprising an amino acid sequence encoded by a nucleic acid that hybridizes under stringent conditions with a nucleic acid having a nucleotide sequence complementary to the nucleotide sequence of SEQ ID NO: 605;
(54αB1) a protein comprising at least one amino acid sequence selected from the group consisting of SEQ ID NOs: 607-611, preferably a protein comprising at least 2, 3, or 4 or all sequences of the amino acid sequences. As referred to above, the amino acid sequence of any of SEQ ID NOs: 591 to 597 may be an amino acid sequence derived therefrom by deletion, substitution, insertion or addition of one or several amino acids;
(54αA2-a) a protein comprising an amino acid sequence with deletion, substitution, insertion or addition of one or several amino acids in SEQ ID NO: 613;
(54αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 613;
(54αA2-c) a protein comprising an amino acid sequence encoded by a nucleotide sequence with deletion, substitution, insertion or addition of one or several nucleotides in SEQ ID NO: 612;
(54αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 612;
(54αA2-c) a protein comprising an amino acid sequence encoded by a nucleic acid that hybridizes under stringent conditions with a nucleic acid having a nucleotide sequence complementary to the nucleotide sequence of SEQ ID NO: 612;
(54αB2) a protein comprising at least one amino acid sequence selected from the group consisting of SEQ ID NOs: 614-616, preferably a protein comprising at least 2, or all sequences of the amino acid sequences. As referred to above, the amino acid sequence of any of SEQ ID NOs: 614 to 616 may be an amino acid sequence derived therefrom by deletion, substitution, insertion or addition of one or several amino acids.

The protein selected from the group consisting of the proteins as defined above in (54αA1-a) to (54αA1-c), (54αB1), (54αA2-a) to (54αA2-c), and (54αB2) can be proteins that are found in a protein spot with a molecular weight of around 5 to 60 kDa, preferably around 10 to 50 kDa, and an isoelectric point of 3.5 to 8.5, preferably 4.0 to 8.0 on gels used in the two-dimensional electrophoresis performed under the conditions described above in the subsection titled “Identification of antigens”.

Epitopes of the aforementioned antigens (54αA1-a) to (54αA1-c) and (54&B1) reside in amino acids 121-135 (SEQ ID NO: 940), amino acids 148-154 (SEQ ID NO: 943) of SEQ ID NO: 606. When the antigen in spot 54α contains a variation in the amino acid sequence of SEQ ID NO: 606, amino acids at positions corresponding to the region of at least one of these epitopes may retain the sequence in SEQ ID NO: 606.

From the viewpoint of improving sensitivity (a degree to which a patient can be diagnosed as being positive) or specificity (a degree to which a healthy subject is not diagnosed as being positive), the antigen in spot 54α may be the following variant.

In the epitope having the sequence of SEQ ID NO: 943, sites of amino acids other than X in KXXXXTX or KXXXXTQ are important for binding to IgE antibodies from allergic patients. Thus, in another preferred mode, when the antigen in spot 54α contains a variation in the amino acid sequence of SEQ ID NO: 606, amino acids 148-154 of SEQ ID NO: 606 in this antigen may have the amino acid sequence of KXXXXTX or KXXXXTQ.

Preferably, a protein that is an antigen in spot 54α has activity equivalent to that of the protein having the amino acid sequence of SEQ ID NO: 606 (superoxide dismutase [Fe], chloroplastic isoform X1) or the protein having the amino acid sequence of SEQ ID NO: 613 (iron-superoxide dismutase) or causative of an allergy to soybean.

### (55α) Antigen in Spot 55α

As the result of sequence identification of the antigen in spot 55α by mass spectroscopy, the amino acid sequences of SEQ ID NOs: 619-649 were detected.

Also, the mass spectroscopic data obtained for spot 55α on a mass spectrometer was analyzed by comparing the data against the NCBI protein data, and as a result, 51 kDa seed maturation protein precursor (amino acid sequence: SEQ ID NO: 618, encoding nucleotide sequence: SEQ ID NO: 617) was identified as a protein comprising any of SEQ ID NOs: 619 to 635 and Lea protein precursor (amino acid sequence: SEQ ID NO: 637, encoding nucleotide sequence: SEQ ID NO: 636) was identified as a protein comprising any of SEQ ID NOs: 638 to 649.

Accordingly, the antigen in spot 55α in the present invention can be any of proteins selected from the group consisting of the proteins as defined below in (55αA1-a) to (55αA1-e), (55αB1), (55αA2-a) to (55αA2-c), and (55αB2):
(55αA1-a) a protein comprising an amino acid sequence with deletion, substitution, insertion or addition of one or several amino acids in SEQ ID NO: 618;
(55αA1-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 618;
(55αA1-c) a protein comprising an amino acid sequence encoded by a nucleotide sequence with deletion, substitution, insertion or addition of one or several nucleotides in SEQ ID NO: 617;
(55αA1-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 617;
(55αA1-e) a protein comprising an amino acid sequence encoded by a nucleic acid that hybridizes under stringent conditions with a nucleic acid having a nucleotide sequence complementary to the nucleotide sequence of SEQ ID NO: 617;
(55αB1) a protein comprising at least one amino acid sequence selected from the group consisting of SEQ ID NOs: 619-635, preferably a protein comprising at least 2, 3, 4, 5, 6, 7, 8, 9, 10, 11, 12, 13, 14, 15, or 16 or all sequences of the amino acid sequences. As referred to above, the amino acid sequence of any of SEQ ID NOs: 619 to 635 may be an amino acid sequence derived therefrom by deletion, substitution, insertion or addition of one or several amino acids;
(55αA2-a) a protein comprising an amino acid sequence with deletion, substitution, insertion or addition of one or several amino acids in SEQ ID NO: 637;
(55αA2-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 637;
(55αA2-c) a protein comprising an amino acid sequence encoded by a nucleotide sequence with deletion, substitution, insertion or addition of one or several nucleotides in SEQ ID NO: 636;
(55αA2-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 636;
(55αA2-c) a protein comprising an amino acid sequence encoded by a nucleic acid that hybridizes under stringent conditions with a nucleic acid having a nucleotide sequence complementary to the nucleotide sequence of SEQ ID NO: 636;
(55αB2) a protein comprising at least one amino acid sequence selected from the group consisting of SEQ ID NOs: 638-649, preferably a protein comprising at least 2, 3, 4, 5, 6, 7, 8, 9, 10, or 11 or all sequences of the amino acid sequences. As referred to above, the amino acid sequence of any of SEQ ID NOs: 638 to 649 may be an amino acid sequence derived therefrom by deletion, substitution, insertion or addition of one or several amino acids.

The protein selected from the group consisting of the proteins as defined above in (55αA1-a) to (55αA1-c), (55αB1), (55αA2-a) to (55αA2-c), and (55αB2) can be proteins that are found in a protein spot with a molecular weight of around 30 to 90 kDa, preferably around 40 to 80 kDa, and an isoelectric point of 5.5 to 10.5, preferably 6.0 to 10.0 on gels used in the two-dimensional electrophoresis performed under the conditions described above in the subsection titled “Identification of antigens”.

Epitopes of the aforementioned antigens (55αA1-a) to (55αA1-c) and (55αB1) reside in amino acids 201-209 (SEQ ID NO: 946), amino acids 318-325 (SEQ ID NO: 949), amino acids 202-210 (SEQ ID NO: 952) of SEQ ID NO: 618. When the antigen in spot 55α contains a variation in the amino acid sequence of SEQ ID NO: 618, amino acids at positions corresponding to the region of at least one of these epitopes may retain the sequence in SEQ ID NO: 618.

From the viewpoint of improving sensitivity (a degree to which a patient can be diagnosed as being positive) or specificity (a degree to which a healthy subject is not diagnosed as being positive), the antigen in spot 55α may be the following variant.

In the epitope having the sequence of SEQ ID NO: 946, sites of amino acids other than X in SEQ ID NO: 944 or 945 are important for binding to IgE antibodies from allergic patients. Thus, in another preferred mode, when the antigen in spot 55α contains a variation in the amino acid sequence of SEQ ID NO: 618, amino acids 201-209 of SEQ ID NO: 618 in this antigen may have the amino acid sequence of SEQ ID NO: 944 or 945.

In the epitope having the sequence of SEQ ID NO: 949, sites of amino acids other than X in SEQ ID NO: 947 or 948 are important for binding to IgE antibodies from allergic patients. Thus, in another preferred mode, when the antigen in spot 55α contains a variation in the amino acid sequence of SEQ ID NO: 618, amino acids 318-325 of SEQ ID NO: 618 in this antigen may have the amino acid sequence of SEQ ID NO: 947 or 948.

In the epitope having the sequence of SEQ ID NO: 952, sites of amino acids other than X in KDXXXXXXQ or SEQ ID NO: 951 are important for binding to IgE antibodies from allergic patients. Thus, in another preferred mode, when the antigen in spot 55α contains a variation in the amino acid sequence of SEQ ID NO: 618, amino acids 202-210 of SEQ ID NO: 618 in this antigen may have the amino acid sequence of KDXXXXXXQ or SEQ ID NO: 951.

Preferably, a protein that is an antigen in spot 55α has activity equivalent to that of the protein having the amino acid sequence of SEQ ID NO: 618 (51 kDa seed maturation protein precursor) or the protein having the amino acid sequence of SEQ ID NO: 637 (Lea protein precursor) or causative of an allergy to soybean.

The proteins that are the aforementioned antigens (1α) to (55α) include those proteins whose amino acid residues are modified by phosphorylation, sugar chain modification, aminoacylation, ring-opening, deamination or the like.

By stating herein “deletion, substitution, insertion or addition of one or several amino acids” in relation to amino acid sequence, it is meant that in an amino acid sequence of interest, one or several amino acids (e.g., 30%, preferably 25%, 20%, 15%, 10%, 5%, 3%, 2% or 1%, of amino acids with respect to the total length of the amino acid sequence, or e.g., 1, 2, 3, 4, 5, 6, 7, 8, 9 or 10 amino acids) are deleted, one or several amino acids are substituted by any other amino acids, any other amino acids are inserted, and/or any other amino acids are added.

Among the aforementioned modifications, substitution is preferably conservative substitution. The “conservative substitution” refers to the substitution of a certain amino acid residue by a different amino acid residue having similar physicochemical characteristics, and can be any type of substitution as long as it does not substantially change the characteristics of the structure of the original sequence for example, any type of substitution is acceptable as long as any substituted amino acids do not disrupt the helical structure of the original sequence or other secondary structures that characterize the original sequence. The following gives examples of separate groups of amino acid residues that are conservatively substitutable with each other, but substitutable amino acid residues are not limited to the examples given below.
Group A: leucine, isoleucine, valine, alanine, methionine
Group B: aspartic acid, glutamic acid
Group C: asparagine, glutamine
Group D: lysine, arginine
Group E: serine, threonine
Group F: phenylalanine, tyrosine

In the case of non-conservative substitution, one member belonging to one of the aforementioned groups can be replaced with a member belong to any other group. For example, in order to eliminate the possibility of unwanted sugar-chain modification, amino acids of group B, D or E as listed above may be substituted by those of any other group. Also, cysteines may be deleted or substituted by any other amino acids to prevent them from being folded into a protein in its tertiary structure. Also, in order to maintain the balance between hydrophilicity and hydrophobicity or to increase hydrophilicity for the purpose of facilitating synthesis, any amino acids may be substituted in consideration of the hydropathy scales of amino acids, which are a measure of the hydrophilic and hydrophobic properties of amino acids (J. Kyte and R. Doolittle, J. Mol. Biol., Vol. 157, p. 105-132, 1982).

As referred to herein, the percent identity between two amino acid sequences can be determined by visual inspection and mathematical calculation. Alternatively, the percent identity can be determined using a computer program. Examples of such computer programs include BLAST, FASTA (Altschul, et al., J. Mol. Biol., 215:403-410 (1990)), and ClustalW. In particular, various conditions (parameters) for identity searches with the BLAST program are described in Altschul, et al. (Nucl. Acids. Res., 25, p.3389-3402, 1997), and are publicly available on the websites of the National Center for Biotechnology Information (NCBI) and DNA Data Bank of Japan (DDBJ) (Altschul, et al., BLAST Manual, Altschul, et al., NCB/NLM/NIH Bethesda, MD 20894). Also, the percent identity can be determined using a genetic information processing software program, such as GENETYX Ver.7 (Genetyx Corporation), DNASIS Pro (Hitachi Software Engineering Co., Ltd.), or Vector NTI (Infomax Inc.).

By stating herein “deletion, substitution, insertion or addition of one or several nucleotides” in relation to nucleotide sequence, it is meant that in a nucleotide sequence of interest, one or several nucleotides (e.g., 30%, preferably 25%, 20%, 15%, 10%, 5%, 3%, 2% or 1%, of nucleotides with respect to the total length of the nucleotide sequence, or e.g., 1, 5, 10, 15, 20, 25 or 30 nucleotides) are deleted, one or several nucleotides are substituted by any other nucleotides, any other nucleotides are inserted, and/or any other nucleotides are added. It is preferable that such a nucleotide deletion, substitution, insertion or addition should not give rise to a frame shift in an amino acid coding sequence.

As referred to herein, the percent identity between two nucleotide sequences can be determined by visual inspection and mathematical calculation. Alternatively, the percent identity can be determined using a computer program. Examples of such sequence comparison computer programs include the BLASTN program, version 2.2.7, available on the website of the National Library of Medicine (http://www.ncbi.nlm.nih.gov/blast/b12seq/bls.html) (Altschul, et al. (1990) J. Mol. Biol., 215:403-10), or the WU-BLAST 2.0 algorithm. Standard default parameter settings for WU-BLAST 2.0 are found and available on the following website: http://blast.wustl.cdu.

As referred to herein, “under stringent conditions” means that hybridization takes place under moderately or highly stringent conditions. To be specific, the moderately stringent conditions can be easily determined by those having ordinary skill in the art on the basis of, for example, the length of DNA. Basic conditions are described in Sambrook, et al., Molecular Cloning: A Laboratory Manual, 3rd ed., ch.6-7, Cold Spring Harbor Laboratory Press, 2001. The moderately stringent conditions include hybridization under the conditions of preferably 1×SSC to 6×SSC at 42° C. to 55° C., more preferably 1×SSC to 3×SSC at 45° C. to 50° C., most preferably 2×SSC at 50° C. In the case of using a hybridization solution containing, for example, about 50% formamide, a temperature around 5 to 15° C. lower than the foregoing should be adopted. Washing is also carried out under the conditions of 0.5×SSC to 6×SSC at 40° C. to 60° C. In the process of hybridization and washing, generally 0.05% to 0.2% SDS, preferably about 0.1% SDS, may be added. Likewise, the highly stringent conditions can be easily determined by those having ordinary skill in the art on the basis of, for example, the length of DNA. Generally, the highly stringent (high stringent) conditions include hybridization and/or washing at a higher temperature and/or a lower salt concentration than those adopted under the moderately stringent conditions. For example, hybridization is carried out under the conditions of 0.1×SSC to 2×SSC at 55° C. to 65° C., more preferably 0.1×SSC to 1×SSC at 60° C. to 65° C., most preferably 0.2×SSC at 63° C. Washing is carried out under the conditions of 0.2×SSC to 2×SSC at 50° C. to 68° C., more preferably 0.2×SSC at 60 to 65° C.

The residue of X as to the epitopes of the aforementioned antigens (1α) to (55) is an amino acid residue at a site where binding activity against IgE antibodies from allergic patients was maintained even after substitution by alanine in alanine scanning described in Example 4. It is well known to those skilled in the art that even when such a site is substituted by any other amino acids, it is highly probable that this binding activity against IgE antibodies is maintained.

Antigens may be obtained by separating and purifying them from soybean using a combination of protein purification methods well known to those skilled in the art. Also, antigens may be obtained by expressing them as recombinant proteins using a genetic recombination technique well known to those skilled in the art and by separating and purifying them using protein purification methods well known to those skilled in the art.

Exemplary protein purification methods include: solubility-based purification methods such as salt precipitation and solvent precipitation; purification methods based on molecular weight difference, such as dialysis, ultrafiltration, gel filtration and SDS-PAGE; charge-based purification methods such as ion exchange chromatography and hydroxylapatite chromatography; specific affinity-based purification methods such as affinity chromatography; purification methods based on hydrophobicity difference, such as reverse-phase high-performance liquid chromatography; and purification methods based on isoelectric point difference, such as isoelectric focusing.

Preparation of a protein by a genetic recombination technique is carried out by preparing an expression vector comprising an antigen-encoding nucleic acid, introducing the expression vector into appropriate host cells by gene transfer or genetic transformation, culturing the host cells under suitable conditions for expression of a recombinant protein, and recovering the recombinant protein expressed in the host cells.

The “vector” refers to a nucleic acid that can be used to introduce a nucleic acid attached thereto into host cells. The “expression vector” is a vector that can induce the expression of a protein encoded by a nucleic acid introduced therethrough. Exemplary vectors include plasmid vectors and viral vectors. Those skilled in the art can select an appropriate expression vector for the expression of a recombinant protein depending on the type of host cells to be used.

The “host cells” refers to cells that undergo gene transfer or genetic transformation by a vector. The host cells can be appropriately selected by those skilled in the art depending on the type of the vector to be used. The host cells can be derived from prokaryotes such as E. coli. When prokaryotic cells like E coli. are used as host cells, the antigen of the present invention may be designed to contain an N-terminal methionine residue in order to facilitate the expression of a recombinant protein in the prokaryotic cells. The N-terminal methionine can be cleaved from the recombinant protein after expression. Also, the host cells may be cells derived from eukaryotes, such as single-cell eukaryotes like yeast, plant cells and animal cells (e.g., human cells, monkey cells, hamster cells, rat cells, murine cells or insect cells) or silkworm.

Gene transfer or genetic transformation of an expression vector into host cells can be carried out as appropriate by following a technique known to those skilled in the art. Those skilled in the art can make possible the expression of a recombinant protein by selecting suitable conditions for the expression of the recombinant protein as appropriate depending on the type of host cells and culturing the host cells under the selected conditions. Then, those skilled in the art can homogenize the host cells having the expressed recombinant protein, and separate and purify an antigen expressed as the recombinant protein from the resulting homogenate by using an appropriate combination of such protein purification methods as mentioned above.

### Diagnosis Kit and Method (1)

The present invention provides a method for providing an indicator for diagnosing an allergy to a soybean in a subject, the method comprising the steps of:
(i) contacting a sample obtained from the subject with an antigen, wherein the sample is a solution comprising an Ig antibody;
(ii) detecting binding between an IgE antibody present in the sample from the subject and the antigen; and
(iii) when the binding between the IgE antibody in the subject and the antigen is detected, an indicator of the fact that the subject is allergic to a soybean is provided;
wherein the antigen is at least one of proteins as defined above in any of (1α) to (55α).

The sample obtained from a subject is a solution containing an Ig antibody, preferably an IgE antibody, as collected from the subject. Examples of such solutions include blood, saliva, sputum, snivel, urine, sweat, and tear. The sample obtained from the subject may be subjected to pretreatment for increasing the concentration of an Ig antibody in the sample before being contacted with an antigen. The pretreatment of a sample may involve, for example, collection of the serum or the plasma from the blood. Furthermore, a Fab moiety that is an antigen-binding moiety may be purified. In a particularly preferred mode, the step (i) mentioned above is carried out by contacting an IgE antibody present in the serum obtained from a subject with an antigen.

The IgE antibody may be the IgE antibody itself or may be mast cells or the like bound to IgE antibodies.

Detection of contact and binding between the sample obtained from a subject and an antigen can be carried out by using a known method. Examples of such methods that can be used include detection by ELISA (Enzyme-Linked Immunosorbent Assay), sandwich immunoassay, immunoblotting, immunoprecipitation, or immunochromatography. These are all techniques for detecting binding between an antigen and an IgE antibody from a subject by contacting and binding the IgE antibody from a subject with the antigen, allowing an enzymatically labelled secondary antibody to act on the IgE antibody specifically bound to the antigen, and adding an enzyme substrate (generally, chromogenic or luminescent reagent) to detect an enzymatic reaction product. Also, a method for detecting a fluorescently labeled secondary antibody can be used. Alternatively, detection by a measurement method capable of evaluating binding between an antigen and an IgE antibody, such as surface plasmon resonance (SPR), can also be used. A plurality of antigen-specific IgE antibodies may be mixed.

The antigen may be provided as an isolated antigen in a state immobilized on a carrier. In this case, the steps (i) and (ii) mentioned above can be carried out using ELISA, sandwich immunoassay, immunochromatography, surface plasmon resonance, or the like. Also, the step (i) mentioned above can be carried out by contacting the sample obtained from a subject with an antigen-immobilized surface. The isolated antigen may be obtained by separating and purifying it from soybean using a combination of protein purification methods well known to those skilled in the art, or by preparing it using a genetic recombination technique. The IgE antibody from the subject may be used in a state immobilized on a carrier, and binding to the antigen may be detected by the aforementioned technique.

The antigen may be in a state unimmobilized on a carrier. In this case, flow cytometry or the like can be used in the aforementioned steps (i) and (ii), and the presence of the antigen bound to the antibody can be confirmed with laser beam. Examples include a basophil activation test (BAT). Another example includes a histamine release test (HRT) which examines whether histamine is released by further contacting an antigen with blood cells in a sample.

The antigen may be detected by immunoblotting in a state separated by two-dimensional electrophoresis. The two-dimensional electrophoresis is a technique for separating a protein sample by performing isoelectric focusing in the first dimension and performing SDS-PAGE (SDS-polyacrylamide gel electrophoresis) in the second dimension. The conditions for two-dimensional electrophoresis are not particularly limited as long as the conditions permit the separation of the antigen of the present invention. For example, the conditions for two-dimensional electrophoresis as described above in the subsection titled “Identification of antigens” can be adopted. Also, the electrophoresis conditions may be defined by reference to the descriptions in Patent Literatures 1 to 4 mentioned above. For example, two-dimensional electrophoresis can be carried out under the conditions that satisfy at least one selected from the group consisting of the following requirements:
(A) the isoelectric focusing gels used in the first dimension should have a gel-strip length of 5 to 10 cm and a gel pH range of 3 to 10, and the pH gradient of the gels in the direction of electrophoresis should be as follows: where the gel-strip length up to pH 5 is taken as “a”, that length from pH 5 to 7 as “b”, and that length above pH 7 as “c”, the relations “a<b” and “b>c” are satisfied;
(B) in the case of (A), when the total gel-strip length is taken as 1, “a” should be in the range of 0.15 to 0.3, “b” should be in the range of 0.4 to 0.7, and “c” should be in the range of 0.15 to 0.3;
(C) in the first dimensional isoelectric focusing, a constant voltage step should be performed by applying a constant voltage ranging from 100 V to 600 V per gel strip containing a sample, and after the electrophoresis variation width during electrophoresis for 30 minutes falls within the range of 5 μA, a voltage-increasing step should be started at which the voltage is increased from the aforementioned constant voltage;
(D) in the case of (C), the final voltage at the voltage-increasing step should be in the range of 3000 V to 6000 V;
(E) the isoelectric focusing gels used in the first dimension should have a longitudinal gel-strip length of 5 to 10 cm, and the electrophoresis gels used in the second dimension should have a gel concentration at the distal end in the direction of electrophoresis, which is in the range of 3 to 6%; and
(F) in the case of (E), the electrophoresis gels used in the second dimension should have a gel concentration at the proximal end in the direction of electrophoresis, which is set to a higher value than that at the distal end in the direction of electrophoresis.

The aforementioned antigens (1α) to (55α) are antigens that specifically bind to IgE antibodies from patients with allergy to soybean. Therefore, when binding between an IgE antibody from a subject and the antigen is detected, an indicator of the fact that the subject is allergic to soybean is provided.

The present invention further provides a kit for diagnosing an allergy to a soybean, comprising at least one of the aforementioned antigens (1α) to (55α). The diagnosis kit of this invention may be used in the aforementioned method for providing an indicator for diagnosing an allergy to a soybean or in a diagnosis method as described later. The diagnosis kit of this invention may comprise not only the at least one of the aforementioned antigens (1α) to (55α), but also an anti-IgE antibody labeled with an enzyme and a chromogenic or luminescent substrate serving as a substrate for the enzyme. Also, a fluorescent-labeled anti-IgE antibody may be used. In the diagnosis kit of this invention, the antigen may be provided in a state immobilized on a carrier. The diagnosis kit of this invention may also be provided together with instructions on the procedure for diagnosis or a package containing said instructions.

In another mode, the aforementioned diagnosis kit comprises a companion diagnostic agent for an allergy to a soybean. The companion diagnostic agent is used for identifying patients expected to respond to pharmaceutical products or identifying patients having the risk of severe adverse reactions to pharmaceutical products, or for studying the reactivity of pharmaceutical products in order to optimize treatment using the pharmaceutical products. Here, the optimization of treatment includes, for example, determination of dosage and administration, judgment regarding discontinuation of administration, and confirmation of an allergen component that is used to cause immunological tolerance.

The present invention further provides a composition for diagnosing an allergy to a soybean, comprising at least one of the aforementioned antigens (1α) to (55α). The diagnosis composition of this invention can be used in a diagnosis method as described below. The diagnosis composition of this invention may further comprise a pharmaceutically acceptable carrier and/or additives commonly used with the antigen of this invention depending on the need.

In one mode, the present invention provides a method for diagnosing an allergy to a soybean in a subject, the method comprising:
(i) contacting a sample obtained from the subject with an antigen;
(ii) detecting binding between an IgE antibody present in the sample from the subject and the antigen; and
(iii) when the binding between the IgE antibody in the subject and the antigen is detected, diagnosing the subject as being allergic to a soybean;
wherein the antigen is at least one of proteins as defined above in any of (1α) to (55α). In this method, the steps (i) and (ii) are performed as described above regarding the corresponding steps of the method for providing an indicator for diagnosing an allergy to a soybean.

In another mode, the present invention provides a method for diagnosing an allergy to a soybean in a subject, the method comprising administering to the subject at least one of the aforementioned antigens (1α) to (55α). This method may be performed in the form of a skin test characterized by applying the antigen onto the skin. Examples of the skin test include various forms of tests, such as: a prick test in which a diagnosis composition is applied onto the skin and then a tiny prick to such an extent as not to provoke bleeding is made in the skin to allow an antigen to penetrate the skin, thereby observing a skin reaction; a scratch test in which a diagnosis composition is applied onto the skin and then the skin is lightly scratched to observe a reaction; a patch test in which a diagnosis composition in the form of cream, ointment, etc. is applied onto the skin to observe a reaction; and an intracutaneous test in which an antigen is administered intracutaneously to observe a reaction. If a skin reaction such as swelling occurs in a skin portion to which the antigen has been applied, the subject of interest is diagnosed as having an allergy to a soybean. The amount of the antigen to be applied to the skin in such tests can be, for example, not more than 100 μg per dose.

In the process of allergy diagnosis, a load test aiming to identify an antigen is often adopted. At least one of the aforementioned antigens (1α) to (55α) can be used as an active ingredient for a load test to diagnose an allergy to a soybean. Here, the antigen protein to be used in the load test may be a protein that has been expressed and purified and may be a protein that has been expressed in food or cooking ingredients, such as rice-based vaccine expressing pollen allergens which are obtained by transforming rice with a gene of a cedar pollen antigen and expressing the antigen protein in the rice.

In yet another mode, the present invention provides at least one of the aforementioned antigens (1α) to (55α), intended for use in the diagnosis of an allergy to a soybean. This also includes the provision of at least one of the aforementioned antigens (1α) to (55α) mixed with a known antigen.

In still another mode, the present invention provides use of at least one of the aforementioned antigens (1α) to (55α) for the production of a diagnostic agent for an allergy to a soybean.

### Pharmaceutical Composition and Treatment Method (1)

The present invention provides a pharmaceutical composition comprising at least one of the aforementioned antigens (1α) to (55α). In one mode, the aforementioned pharmaceutical composition is used for the treatment and diagnosis of an allergy to a soybean.

The present invention also provides a method for treating an allergy to a soybean, the method comprising administering at least one of the aforementioned antigens (1α) to (55α) to a patient in need of a treatment for an alle-rgy to a soybean.

In another mode, the present invention provides at least one of the aforementioned antigens (1α) to (55α), intended for use in the treatment for an allergy to a soybean. In yet another mode, the present invention provides use of at least one of the aforementioned antigens (1α) to (55α) for the production of a therapeutic agent for an allergy to a soybean.

In the process of allergy treatment, a hyposensitization therapy aiming to induce immunological tolerance by administering an antigen to a patient is often adopted. The at least one of the aforementioned antigens (1α) to (55α) can be used as an active ingredient for a hyposensitization therapy for an allergy to a soybean. Here, the antigen protein to be used in the hyposensitization therapy may be a protein that has been expressed and purified and may be a protein that has been expressed in food or cooking ingredients, such as rice-based vaccine expressing pollen allergens which are obtained by transforming rice with a gene of a cedar pollen antigen and expressing the antigen protein in the rice.

The pharmaceutical composition of the present invention can be administered by common administration routes. Examples of common administration routes include oral, sublingual, percutaneous, intracutaneous, subcutaneous, intravascular, intranasal, intramuscular, intraperitoneal, and intrarectal administrations.

The pharmaceutical composition of the present invention can be used as a pharmaceutical composition to which a commonly used pharmaceutically acceptable adjuvant or excipient or any other additives (e.g., stabilizer, solubilizer, emulsifier, buffer, preservative, colorant) are added by a conventional method together with the antigen of this invention depending on the need. The dosage form of the pharmaceutical composition can be selected by those skilled in the art as appropriate depending on the administration route. The pharmaceutical composition can be in the form of, for example, tablet, capsule, troche, sublingual tablet, parenteral injection, intranasal spray, poultice, solution, cream, or lotion. The administration dose, frequency and/or period of the pharmaceutical composition of this invention can be selected by a physician as appropriate depending on the administration route and the patient's condition and characteristics such as age and body weight. For example, the pharmaceutical composition may be administered to an adult patient at a dose of not more than 100 μg per dose. The administration interval can be, for example, once daily, once weekly, twice weekly, once per three months or so. The administration period can be, for example, several weeks to several years. The pharmaceutical composition may be administered in such a manner that the dose is increased in incremental steps over the administration period.

### Tester (1)

The present invention provides a tester comprising an antibody for at least one of the aforementioned antigens (1α) to (55α).

The antibody can be prepared by a conventional method. For example, the antibody may be prepared by immunizing a mammal such as rabbit with one of the aforementioned antigens (1α) to (55α). The antibody may be an Ig antibody, a polyclonal antibody, a monoclonal antibody, or an antigen-binding fragment thereof (e.g., Fab, F(ab′) 2, Fab′).

Further, in the aforementioned tester, the antibody may be provided in a form bound to a carrier. The type of the carrier is not particularly limited as long as it is usable for detection of binding between an antibody and an antigen. Any given carrier known to those skilled in the art can be used.

Examples of a method for determining the presence or absence of an antigen include the following methods:
a method in which a prepared tester comprising an IgE antibody is contacted with a sample obtained from a food, a cooking ingredient, etc., ELISA or the like method is used to detect whether there is a binding between the IgE antibody and an antigen in the sample, and if the binding between the IgE antibody and the antigen is detected, it is determined that the antibody remains in the food, the cooking ingredient, etc. of interest; and
a method in which filter paper is impregnated with a food or a cooking ingredient and reacted with an antibody solution so as to detect an antigen contained therein.

Another mode of the present invention includes a tester for determining the presence or absence of an antigen of an allergy to a soybean in an object of interest, the tester comprising a primer having a nucleotide sequence complementary to a portion of at least one of the nucleotide sequences of SEQ ID NOs: 1, 3, 5, 7, 9, 11, 13, 15, 69, 88, 105, 121, 138, 154, 158, 162, 168, 173, 188, 200, 203, 219, 232, 238, 241, 253, 279, 285, 290, 305, 319, 326, 333, 340, 347, 357, 366, 383, 393, 410, 417, 421, 428, 434, 440, 446, 452, 455, 459, 467, 472, 478, 484, 488, 494, 514, 517, 521, 524, 532, 540, 546, 552, 555, 559, 564, 571, 577, 583, 589, 598, 605, 612, 617 and 636. The primer has, for example, but not limited to, a nucleotide sequence complementary to, preferably, 12 residues, 15 bases, 20 bases, or 25 bases, in a 3′-terminal or central sequence in a partial sequence of at least one of the nucleotide sequences of SEQ ID NOs defined above in this paragraph. Particularly, when mRNA is of interest, the tester has a complementary primer of a poly-A tail. In a preferred mode, the tester comprising the primer mentioned above may further comprise a primer comprising a 5′-terminal nucleotide sequence, preferably a nucleotide sequence of 12 bases, 15 bases, 20 bases, or 25 bases, of at least one of the nucleotide sequences of SEQ ID NOs defined above in this paragraph.

For example, cDNA is amplified by PCR (Polymerase Chain Reaction) including RT-PCR using templated DNA or mRNA obtained from a soybean and the aforementioned complementary primer, and the sequence of the amplified cDNA is compared with the nucleotide sequences of SEQ ID NOs defined above in the previous paragraph to determine the presence or absence of the antigen. Amplification methods by PCR can be exemplified by RACE. In this respect, even if there exists a point mutation encoding the same amino acid in the comparison of the amplified cDNA with the nucleotide sequences of SEQ ID NOs defined above in the previous paragraph, or even if the nucleotide sequence of the amplified cDNA has insertion, deletion, substitution or addition of bases in the nucleotide sequences of SEQ ID NOS defined above in the previous paragraph, it is determined that the antigen is present when the amino acid sequence encoded by the cDNA has at least 70%, preferably at least 80, 90, 95, 98, or 99% identity to the amino acid sequence of SEQ ID NO: 2, 4, 6, 8, 10, 12, 14, 16, 70, 89, 106, 122, 139, 155, 159, 163, 169, 174, 189, 201, 204, 220, 233, 239, 242, 254, 280, 286, 291, 306, 320, 327, 334, 341, 348, 358, 367, 384, 394, 411, 418, 422, 429, 435, 441, 447, 453, 456, 460, 468, 473, 479, 485, 489, 495, 515, 518, 522, 525, 533, 541, 547, 553, 556, 560, 565, 572, 578, 584, 590, 599, 606, 613, 618 or 637.

In one mode, the aforementioned tester is used to determine the presence or absence of an antigen in cooking ingredients or in products of interest in a food production line. The tester may also be used for quality inspection of production lines and pre-shipment products by manufacturers, or may be used for self-checking of the presence or absence of an antigen in a food or cooking ingredients of interest by consumers.

### Allergen-Free Food and the Like (1)

The present invention provides a soybean or processed product of soybean in which at least one of the aforementioned antigens (1α) to (55α) is eliminated or reduced.

The method for eliminating or reducing the antigen of the present invention in a soybean or processed products of soybean is not limited. The elimination or reduction of the antigen may be conducted by any method, as long as the method permits the elimination or reduction of the antigen.

For example, the soybean of the present invention whose antigen is eliminated or reduced may be obtained by preparing a soybean in which the expression of the antigen of the present invention is knocked out, using a gene knock-out technique. Any technique known to those skilled in the art such as genetic modification can be used as the gene knock-out technique.

An antigen of the present invention may be the artefact that an antigen of the present invention assumed the removal or a reduced soybean raw material as for the removal or the reduced processed products of soybean. In the case of using an ordinary soybean as a source ingredient, a treatment for removing or reducing the antigen of this invention is performed before or after preparation of a processed product of soybean. The methods for removing or reducing the antigen of the present invention in the processed products of soybean which assumed an ordinary soybean raw material include a method to remove protein component in food such as a high pressure treatment and elution with the neutral salt solution or the high temperature steam, and a method to perform hydrolysis, denaturation, or amino acid alteration (chemical modification, elimination, or the like of a side chain) by heat treatment and acid treatment.

### Method for Producing Antigen-Free Processed Product of Soybean

The present invention provides a method for producing a processed product of soybean in which an antigen is eliminated or reduced, the method comprising the step of confirming that the antigen is eliminated or reduced, in a production process of the processed product, wherein the antigen is at least one of the aforementioned antigens (1) to (55α).

The step of confirming that the antigen is eliminated or reduced, in a production process of the processed product of soybean in which an antigen is eliminated or reduced may be performed by confirming the presence or absence of an antigen by the method described above in the subsection titled “Tester (1)”.

The production of the processed product of soybean in which an antigen is eliminated or reduced may be performed by the method described above in the subsection titled “Allergen-free food and the like (1)”.

### Epitope of Antigen

Epitopes and amino acids important for binding activity against IgE antibodies from allergic patients within the epitopes were identified as shown in Examples 4 and 5 as to antigens identified as shown in Example 3 and known antigens Gly m 4, Gly m 5, Gly m 6 and Gly m 8.

The present invention provides polypeptides of the following (1β) to (60β) as polypeptides comprising an amino acid sequence specifically binding to an IgE antibody from an allergic patient.

### (1β) Epitope of Endoplasmin Homolog Isoform X2 (Spot 1α)

In the present invention, the polypeptide (1β) can be any polypeptide selected from the group consisting of (1β-1) to (1β-4) as defined below:
(1β-1) a polypeptide comprising at least one amino acid sequence selected from the group consisting of SEQ ID NOs: 652, 655 and 657-660;
(1β-2) a polypeptide comprising the amino acid sequence of XXKXEXXX, preferably a polypeptide comprising the amino acid sequence of XTKXEXXX, more preferably a polypeptide comprising an amino acid sequence in which any 1, 2, 3, 4 or 5 of the amino acids at positions 1, 2, 4, and 6-8 of SEQ ID NO: 652 are substituted by alanine, further preferably a polypeptide comprising an amino acid sequence in which any 1, 2, 3 or 4 of the amino acids at positions 1, 4, and 6-8 of SEQ ID NO: 652 are substituted by other amino acids;
(1-3) a polypeptide comprising the amino acid sequence of XXXKDNVXX (SEQ ID NO: 653), preferably a polypeptide comprising the amino acid sequence of XLXKDNVXX (SEQ ID NO: 654), more preferably a polypeptide comprising an amino acid sequence in which any 1, 2, 3, 4 or 5 of the amino acids at positions 1-3, 8, and 9 of SEQ ID NO: 655 are substituted by alanine, further preferably a polypeptide comprising an amino acid sequence in which any 1, 2, 3 or 4 of the amino acids at positions 1, 3, 8, and 9 of SEQ ID NO: 655 are substituted by other amino acids; and
(1β-4) a polypeptide comprising the amino acid sequence of EEEDDXXXXXEXXXX (SEQ ID NO: 656), more preferably a polypeptide comprising an amino acid sequence in which any 1, 2, 3, 4, 5, 6, 7, 8 or 9 of the amino acids at positions 6-10 and 12-15 of SEQ ID NO: 657 are substituted by alanine, further preferably a polypeptide comprising an amino acid sequence in which any 1, 2, 3, 4, 5, 6, 7, 8 or 9 of the amino acids at positions 6-10 and 12-15 of SEQ ID NO: 657 are substituted by other amino acids.

### (2β) Epitope of Heat Shock Cognate Protein 80 (Spot 2α)

In the present invention, the polypeptide (2β) can be any polypeptide selected from the group consisting of (2β-1) to (2β-9) as defined below:
(2β-1) a polypeptide comprising at least one amino acid sequence selected from the group consisting of SEQ ID NOs: 663, 666, 669, 670-672, 675, 676-678, 681, 684, 687, 689 and 690-692;
(2-2) a polypeptide comprising the amino acid sequence of DXXDKXXXX, preferably a polypeptide comprising the amino acid sequence of DXXDKXRXX (SEQ ID NO: 662), more preferably a polypeptide comprising an amino acid sequence in which any 1, 2, 3, 4, 5 or 6 of the amino acids at positions 2, 3, and 6-9 of SEQ ID NO: 663 are substituted by alanine, further preferably a polypeptide comprising an amino acid sequence in which any 1, 2, 3, 4 or 5 of the amino acids at positions 2, 3, 6, 8, and 9 of SEQ ID NO: 663 are substituted by other amino acids;
(2β-3) a polypeptide comprising the amino acid sequence of XXXXKSKLXXXXXXX (SEQ ID NO: 664), preferably a polypeptide comprising the amino acid sequence of XXXXKSKLXXXPXXX (SEQ ID NO: 665), more preferably a polypeptide comprising an amino acid sequence in which any 1, 2, 3, 4, 5, 6, 7, 8, 9, 10 or 11 of the amino acids at positions 1-4, and 9-15 of SEQ ID NO: 666 are substituted by alanine, further preferably a polypeptide comprising an amino acid sequence in which any 1, 2, 3, 4, 5, 6, 7, 8, 9 or 10 of the amino acids at positions 1-4, 9-11, and 13-15 of SEQ ID NO: 666 are substituted by other amino acids;
(2β-4) a polypeptide comprising the amino acid sequence of XPDKX, preferably a polypeptide comprising the amino acid sequence of IPDKX (SEQ ID NO: 668), more preferably a polypeptide comprising an amino acid sequence in which any 1 or 2 of the amino acids at positions 1 and 5 of SEQ ID NO: 669 are substituted by alanine, further preferably a polypeptide comprising an amino acid sequence in which any 1 or 2 of the amino acids at positions 1 and 5 of SEQ ID NO: 669 are substituted by other amino acids;
(2β-5) a polypeptide comprising the amino acid sequence of XXFSKXXXXXXXEDX (SEQ ID NO: 673), preferably a polypeptide comprising the amino acid sequence of XXFSKXXXXXXXEDS (SEQ ID NO: 674), more preferably a polypeptide comprising an amino acid sequence in which any 1, 2, 3, 4, 5, 6, 7, 8, 9 or 10 of the amino acids at positions 1, 2, 6-12 and 15 of SEQ ID NO: 675 are substituted by alanine, further preferably a polypeptide comprising an amino acid sequence in which any 1, 2, 3, 4, 5, 6, 7, 8, or 9 of the amino acids at positions 1, 2 and 6-12 of SEQ ID NO: 675 are substituted by other amino acids;
(2β-6) a polypeptide comprising the amino acid sequence of XXXXDXXXXEXXXDX, preferably a polypeptide comprising the amino acid sequence of XXXXDKIRXEXXXDK (SEQ ID NO: 680), more preferably a polypeptide comprising an amino acid sequence in which any 1, 2, 3, 4, 5, 6, 7, 8, 9, 10, 11 or 12 of the amino acids at positions 1-4, 6-9, 11-13 and 15 of SEQ ID NO: 681 are substituted by alanine, further preferably a polypeptide comprising an amino acid sequence in which any 1, 2, 3, 4, 5, 6, 7 or 8 of the amino acids at positions 1-4, 9, 11-13 of SEQ ID NO: 681 are substituted by other amino acids;
(2β-7) a polypeptide comprising the amino acid sequence of XYXXRXXXXQNXXXX (SEQ ID NO: 682), preferably a polypeptide comprising an amino acid sequence in which any 1, 2, 3, 4, 5, 6, 7, 8, 9, 10 or 11 of the amino acids at positions 1, 3, 4, 6-9, and 12-15 of SEQ ID NO: 684 are substituted by alanine, more preferably a polypeptide comprising an amino acid sequence in which any 1, 2, 3, 4, 5, 6, 7, 8, 9, 10 or 11 of the amino acids at positions 1, 3, 4, 6-9, and 12-15 of SEQ ID NO: 684 are substituted by other amino acids;
(2β-8) a polypeptide comprising the amino acid sequence of DXXXRXXXXQNXXXX (SEQ ID NO: 685), preferably a polypeptide comprising an amino acid sequence in which any 1, 2, 3, 4, 5, 6, 7, 8, 9, 10 or 11 of the amino acids at positions 2-4, 6-9, and 12-15 of SEQ ID NO: 687 are substituted by alanine, more preferably a polypeptide comprising an amino acid sequence in which any 1, 2, 3, 4, 5, 6, 7, 8, 9, 10 or 11 of the amino acids at positions 2-4, 6-9, and 12-15 of SEQ ID NO: 687 are substituted by other amino acids;
(2β-9) a polypeptide comprising the amino acid sequence of RKPXXXXXXXXAAXX (SEQ ID NO: 688), preferably a polypeptide comprising an amino acid sequence in which any 1, 2, 3, 4, 5, 6, 7, 8, 9, or 10 of the amino acids at positions 4-11, 14 and 15 of SEQ ID NO: 689 are substituted by alanine, more preferably a polypeptide comprising an amino acid sequence in which any 1, 2, 3, 4, 5, 6, 7, 8, 9, or 10 of the amino acids at positions 4-11, 14 and 15 of SEQ ID NO: 689 are substituted by other amino acids.

### (3β) Epitope of Cell Division Cycle Protein 48 Homolog (Spot 3α)

In the present invention, the polypeptide (3β) can be any polypeptide selected from the group consisting of (3β-1) to (3β-4) as defined below:

(3β-1) a polypeptide comprising at least one amino acid sequence selected from the group consisting of SEQ ID NOs: 695, 697, 699 and 700-716;
(3β-2) a polypeptide comprising the amino acid sequence of XXXXXXXXXXRLXEX, preferably a polypeptide comprising the amino acid sequence of XXXLKREDEERLDEV (SEQ ID NO: 694), more preferably a polypeptide comprising an amino acid sequence in which any 1, 2, 3, 4, 5, 6, 7, 8, 9, 10, 11 or 12 of the amino acids at positions 1-10, 13 and 15 of SEQ ID NO: 695 are substituted by alanine, further preferably a polypeptide comprising an amino acid sequence in which any 1, 2 or 3 of the amino acids at positions 1-3 of SEQ ID NO: 695 are substituted by other amino acids;
(3β-3) a polypeptide comprising the amino acid sequence of XXXXXXXXXXXXXDE, preferably a polypeptide comprising an amino acid sequence in which any 1, 2, 3, 4, 5, 6, 7, 8, 9, 10, 11, 12 or 13 of the amino acids at positions 1-13 of SEQ ID NO: 697 are substituted by alanine, more preferably a polypeptide comprising an amino acid sequence in which any 1, 2, 3, 4, 5, 6, 7, 8, 9, 10, 11, 12 or 13 of the amino acids at positions 1-13 of SEQ ID NO: 697 are substituted by other amino acids;
(3β-4) a polypeptide comprising the amino acid sequence of XXXXGDR, preferably a polypeptide comprising an amino acid sequence in which any 1, 2, 3 or 4 of the amino acids at positions 1˜4 of SEQ ID NO: 699 are substituted by alanine, more preferably a polypeptide comprising an amino acid sequence in which any 1, 2, 3 or 4 of the amino acids at positions 1-4 of SEQ ID NO: 699 are substituted by other amino acids.

In one mode, the polypeptide (3β) may not comprise the full-length amino acid sequence (SEQ ID NO: 16) of Cell division cycle protein 48 homolog or a variant or a homolog having 100% or at least 90%, at least 80% or at least 70% identity to the total length of this amino acid sequence.

SEQ ID NOs: 695, 697, 699 and 700-716 correspond to amino acids 191-205 (SEQ ID NO: 695), amino acids 21-35 (SEQ ID NO: 697), amino acids 779-785 (SEQ ID NO: 699), amino acids 8-14 (SEQ ID NO: 700), amino acids 49-56 (SEQ ID NO: 701), amino acids 101-107 (SEQ ID NO: 702), amino acids 161-175 (SEQ ID NO: 703), amino acids 291-305 (SEQ ID NO: 704), amino acids 316-323 (SEQ ID NO: 705), amino acids 327-333 (SEQ ID NO: 706), amino acids 341-355 (SEQ ID NO: 707), amino acids 361-369 (SEQ ID NO: 708), amino acids 431-445 (SEQ ID NO: 709), amino acids 451-465 (SEQ ID NO: 710), amino acids 501-515 (SEQ ID NO: 711), amino acids 565-572 (SEQ ID NO: 712), amino acids 631-645 (SEQ ID NO: 713), amino acids 703-712 (SEQ ID NO: 714), amino acids 711-715 (SEQ ID NO: 715), and amino acids 721-735 (SEQ ID NO: 716), respectively, of SEQ ID NO: 16.

### (4β) Epitope of Embryo-Specific Urease Isoform X1 (Spot 4α)

In the present invention, the polypeptide (4β) can be any polypeptide selected from the group consisting of (4β-1) to (4β-5) as defined below:
(4β-1) a polypeptide comprising at least one amino acid sequence selected from the group consisting of SEQ ID NOs: 719, 722, 725, 726-728 and 731;
(4β-2) a polypeptide comprising the amino acid sequence of XXXXXXXKED, preferably a polypeptide comprising the amino acid sequence of XXXXXEXKED (SEQ ID NO: 718), more preferably a polypeptide comprising an amino acid sequence in which any 1, 2, 3, 4, 5, 6 or 7 of the amino acids at positions 1-7 of SEQ ID NO: 719 are substituted by alanine, further preferably a polypeptide comprising an amino acid sequence in which any 1, 2, 3, 4, 5 or 6 of the amino acids at positions 1-5 and 7 of SEQ ID NO: 719 are substituted by other amino acids;
(4β-3) a polypeptide comprising the amino acid sequence of KXXLGDX (SEQ ID NO: 720), preferably a polypeptide comprising the amino acid sequence of KIXLGDX (SEQ ID NO: 721), more preferably a polypeptide comprising an amino acid sequence in which any 1, 2 or 3 of the amino acids at positions 2, 3 and 7 of SEQ ID NO: 722 are substituted by alanine, further preferably a polypeptide comprising an amino acid sequence in which any 1, 2 or 3 of the amino acids at positions 3 and 7 of SEQ ID NO: 722 are substituted by other amino acids;
(4β-4) a polypeptide comprising the amino acid sequence of XXXDXFXKXE (SEQ ID NO: 723), preferably a polypeptide comprising the amino acid sequence of XXXDXFXKIE (SEQ ID NO: 724), more preferably a polypeptide comprising an amino acid sequence in which any 1, 2, 3, 4, 5 or 6 of the amino acids at positions 1-3, 5, 7 and 9 of SEQ ID NO: 725 are substituted by alanine, further preferably a polypeptide comprising an amino acid sequence in which any 1, 2, 3, 4 or 5 of the amino acids at positions 1-3, 5 and 7 of SEQ ID NO: 725 are substituted by other amino acids;
(4β-5) a polypeptide comprising the amino acid sequence of XXXHXLXXXNKXXEA (SEQ ID NO: 729), preferably a polypeptide comprising the amino acid sequence of XXVHXLXXXNKXXEA (SEQ ID NO: 730), more preferably a polypeptide comprising an amino acid sequence in which any 1, 2, 3, 4, 5, 6, 7, 8 or 9 of the amino acids at positions 1-3, 5, 7-9,12 and 13 of SEQ ID NO: 731 are substituted by alanine, further preferably a polypeptide comprising an amino acid sequence in which any 1, 2, 3, 4, 5, 6, 7 or 8 of the amino acids at positions 1, 2, 5, 7-9, 12 and 13 of SEQ ID NO: 731 are substituted by other amino acids.

### (53) Alpha-1,4 Glucan Phosphorylase L Isozyme, Chloroplastic/Amyloplastic (Spot 5α)

In the present invention, the polypeptide (5β) can be any polypeptide selected from the group consisting of (5β-1) to (5β-2) as defined below:
(5β-1) a polypeptide comprising at least one amino acid sequence selected from the group consisting of SEQ ID NOs: 734 and 735-738;
(5β-2) a polypeptide comprising the amino acid sequence of XXXXXXGLXKXRXEX (SEQ ID NO: 732), preferably a polypeptide comprising the amino acid sequence of XXXXXXGLXKXRXEG (SEQ ID NO: 733), more preferably a polypeptide comprising an amino acid sequence in which any 1, 2, 3, 4, 5, 6, 8, 9 or 10 of the amino acids at positions 1-6, 9, 11, 13 and 15 of SEQ ID NO: 734 are substituted by alanine, further preferably a polypeptide comprising an amino acid sequence in which any 1, 2, 3, 4, 5, 6, 7, 8 or 9 of the amino acids at positions 1-6, 9, 11 and 13 of SEQ ID NO: 734 are substituted by other amino acids.

### (6β) Epitope of Aconitate Hydratase 1 (Spot 6α)

In the present invention, the polypeptide (6β) can be a polypeptide comprising at least one amino acid sequence selected from the group consisting of SEQ ID NOs: 739 and 740.

### (7β) Epitope of Methionine Synthase (Spot 7α)

In the present invention, the polypeptide (7β) can be any polypeptide selected from the group consisting of (7β-1) to (7β-2) as defined below:
(7β-1) a polypeptide comprising at least one amino acid sequence selected from the group consisting of SEQ ID NOs: 741 and 744;
(7β-2) a polypeptide comprising the amino acid sequence of XXXXXXXKDXVEDXE (SEQ ID NO: 742), preferably a polypeptide comprising the amino acid sequence of XXXXXXXKDEVEDXE (SEQ ID NO: 743), more preferably a polypeptide comprising an amino acid sequence in which any 1, 2, 3, 4, 5, 6, 7, 8 or 9 of the amino acids at positions 1-7, 10 and 14 of SEQ ID NO: 744 are substituted by alanine, further preferably a polypeptide comprising an amino acid sequence in which any 1, 2, 3, 4, 5, 6, 7 or 8 of the amino acids at positions 1-7 and 14 of SEQ ID NO: 744 are substituted by other amino acids.
   (9β) Epitope of Lysine—tRNA Ligase, Cytoplasmic-Like Isoform X2 (Spot 9α)

In the present invention, the polypeptide (9β) can be any polypeptide selected from the group consisting of (9β-1) to (9β-3) as defined below:
(9β-1) a polypeptide comprising at least one amino acid sequence selected from the group consisting of SEQ ID NOs: 747 and 749;
(9β-2) a polypeptide comprising the amino acid sequence of XXEXXXXKDX, preferably a polypeptide comprising the amino acid sequence of NXEXXXXKDX (SEQ ID NO: 746), more preferably a polypeptide comprising an amino acid sequence in which any 1, 2, 3, 4, 5, 6 or 7 of the amino acids at positions 1, 2, 4-7 and 10 of SEQ ID NO: 747 are substituted by alanine, further preferably a polypeptide comprising an amino acid sequence in which any 1, 2, 3, 4, 5 or 6 of the amino acids at positions 2, 4-7 and 10 of SEQ ID NO: 747 are substituted by other amino acids;
(9-3) a polypeptide comprising the amino acid sequence of LDFLEXET (SEQ ID NO: 748), preferably a polypeptide comprising an amino acid sequence in which the amino acid at position 6 of SEQ ID NO: 749 is substituted by alanine, more preferably a polypeptide comprising an amino acid sequence in which the amino acid at position 6 of SEQ ID NO: 749 is substituted by another amino acid.

### (11β) Epitope of Polyadenylate-Binding Protein 8 Isoform X3 (Spot 11α)

In the present invention, the polypeptide (11β) can be any polypeptide selected from the group consisting of (11β-1) to (11β-2) as defined below:
(11β-1) a polypeptide comprising at least one amino acid sequence selected from the group consisting of SEQ ID NOs: 753 and 754;
(11β-2) a polypeptide comprising the amino acid sequence of PLXQQXXXXX (SEQ ID NO: 751), preferably a polypeptide comprising the amino acid sequence of PLXQQGXXXX (SEQ ID NO: 752), more preferably a polypeptide comprising an amino acid sequence in which any 1, 2, 3, 4, 5 or 6 of the amino acids at positions 3 and 6-10 of SEQ ID NO: 753 are substituted by alanine, further preferably a polypeptide comprising an amino acid sequence in which any 1, 2, 3, 4 or 5 of the amino acids at positions 3 and 7-10 of SEQ ID NO: 753 are substituted by other amino acids.

### (12β) Epitope of Pyrophosphate—Fructose 6-Phosphate 1-Phosphotransferase Subunit Alpha-Like (Spot 12α)

In the present invention, the polypeptide (12) can be a polypeptide comprising the amino acid sequence of SEQ ID NO: 755.

### (13β) Epitope of Protein Disulfide Isomerase-Like Protein Precursor (Spot 13α)

In the present invention, the polypeptide (13β) can be any polypeptide selected from the group consisting of (13β-1) to (13β-2) as defined below:
(13-1) a polypeptide comprising the amino acid sequence of SEQ ID NO: 758;
(13β-2) a polypeptide comprising the amino acid sequence of XXGXXXXXXXXXXXE, preferably a polypeptide comprising the amino acid sequence of XXGXLXXXXGXRTXE (SEQ ID NO: 757), more preferably a polypeptide comprising an amino acid sequence in which any 1, 2, 3, 4, 5, 6, 7, 8, 9, 10, 11, 12 or 13 of the amino acids at positions 1, 2 and 4-14 of SEQ ID NO: 758 are substituted by alanine, further preferably a polypeptide comprising an amino acid sequence in which any 1, 2, 3, 4, 5, 6, 7, 8 or 9 of the amino acids at positions 1, 2, 4, 6-9, 11 and 14 of SEQ ID NO: 758 are substituted by other amino acids.

### (14β) Epitope of V-Type Proton ATPase Subunit B 2 (Spot 14α)

In the present invention, the polypeptide (14β) can be any polypeptide selected from the group consisting of (14β-1) to (14β-3) as defined below:
(14β-1) a polypeptide comprising at least one amino acid sequence selected from the group consisting of SEQ ID NOs: 761 and 764;
(14β-2) a polypeptide comprising the amino acid sequence of XXXXXLLXXXXEEDN (SEQ ID NO: 759), preferably a polypeptide comprising the amino acid sequence of XXXXXLLEXXXEEDN (SEQ ID NO: 760), more preferably a polypeptide comprising an amino acid sequence in which any 1, 2, 3, 4, 5, 6, 7, 8 or 9 of the amino acids at positions 1-5 and 8-11 of SEQ ID NO: 761 are substituted by alanine, further preferably a polypeptide comprising an amino acid sequence in which any 1, 2, 3, 4, 5, 6, 7 or 8 of the amino acids at positions 1-5 and 9-11 of SEQ ID NO: 761 are substituted by other amino acids;
(14β-3) a polypeptide comprising the amino acid sequence of RKXXXXXXXXXYXNX (SEQ ID NO: 762), preferably a polypeptide comprising the amino acid sequence of RKDHSXXXXXLYANX (SEQ ID NO: 763), more preferably a polypeptide comprising an amino acid sequence in which any 1, 2, 3, 4, 5, 6, 7, 8, 9, 10 or 11 of the amino acids at positions 3-11, 13 and 15 of SEQ ID NO: 764 are substituted by alanine, further preferably a polypeptide comprising an amino acid sequence in which any 1, 2, 3, 4, 5 or 6 of the amino acids at positions 6-10 and 15 of SEQ ID NO: 764 are substituted by other amino acids.

### (15β) Epitope of UTP-Glucose-1-Phosphate Uridylyltransferase (Spot 15α, 16α)

In the present invention, the polypeptide (15β) can be any polypeptide selected from the group consisting of (15β-1) to (15β-2) as defined below:
(15-1) a polypeptide comprising the amino acid sequence of SEQ ID NO: 766;
(15β-2) a polypeptide comprising the amino acid sequence of XGKXXXXXXXXXXXX, preferably a polypeptide comprising an amino acid sequence in which any 1, 2, 3, 4, 5, 6, 7, 8, 9, 10, 11, 12 or 13 of the amino acids at positions 1 and 4-15 of SEQ ID NO: 766 are substituted by alanine, more preferably a polypeptide comprising an amino acid sequence in which any 1, 2, 3, 4, 5, 6, 7, 8, 9, 10, 11, 12 or 13 of the amino acids at positions 1 and 4-15 of SEQ ID NO: 766 are substituted by other amino acids.

### (17) Epitope of Guanosine Nucleotide Diphosphate Dissociation Inhibitor 2 (Spot 17α)

In the present invention, the polypeptide (17β) can be a polypeptide comprising the amino acid sequence of SEQ ID NO: 767.

### (18) Epitope of Hypothetical Protein GLYMA 10G028300 (Spot 18α)

In the present invention, the polypeptide (18β) can be any polypeptide selected from the group consisting of (18β-1) to (18β-3) as defined below:
(18β-1) a polypeptide comprising at least one amino acid sequence selected from the group consisting of SEQ ID NOs: 750, 768, 771, 772 and 775;
(18β-2) a polypeptide comprising the amino acid sequence of XXXXXXXNXXIFEED (SEQ ID NO: 769), preferably a polypeptide comprising the amino acid sequence of XXXXXXXNXYIFEED (SEQ ID NO: 770), more preferably a polypeptide comprising an amino acid sequence in which any 1, 2, 3, 4, 5, 6, 7, 8 or 9 of the amino acids at positions 1-7, 9 and 10 of SEQ ID NO: 771 are substituted by alanine, further preferably a polypeptide comprising an amino acid sequence in which any 1, 2, 3, 4, 5, 6, 7 or 8 of the amino acids at positions 1-7 and 9 of SEQ ID NO: 771 are substituted by other amino acids;
(18β-3) a polypeptide comprising the amino acid sequence of XELPREXRXX (SEQ ID NO: 773), preferably a polypeptide comprising the amino acid sequence of XELPREERXX (SEQ ID NO: 774), more preferably a polypeptide comprising an amino acid sequence in which any 1, 2, 3 or 4 of the amino acids at positions 1, 7, 9 and 10 of SEQ ID NO: 775 are substituted by alanine, further preferably a polypeptide comprising an amino acid sequence in which any 1, 2 or 3 of the amino acids at positions 1, 9 and 10 of SEQ ID NO: 775 are substituted by other amino acids.

### (19β) Epitope of Sucrose Binding Protein Homolog S-64 (Spot 19α)

In the present invention, the polypeptide (19β) can be any polypeptide selected from the group consisting of (19β-1) to (19β-5) as defined below:
(19β-1) a polypeptide comprising at least one amino acid sequence selected from the group consisting of SEQ ID NOs: 778, 781, 784, 786 and 787;
(19-2) a polypeptide comprising the amino acid sequence of XXXXXPLYXXXXDEX (SEQ ID NO: 776), preferably a polypeptide comprising the amino acid sequence of XXXXXPLYIXXXDEX (SEQ ID NO: 777), more preferably a polypeptide comprising an amino acid sequence in which any 1, 2, 3, 4, 5, 6, 7, 8, 9 or 10 of the amino acids at positions 1-5, 9-12 and 15 of SEQ ID NO: 778 are substituted by alanine, further preferably a polypeptide comprising an amino acid sequence in which any 1, 2, 3, 4, 5, 6, 7, 8 or 9 of the amino acids at positions 1-5, 10-12 and 15 of SEQ ID NO: 778 are substituted by other amino acids;
(19β-3) a polypeptide comprising the amino acid sequence of XXXXXXXXRXRRXXX, preferably a polypeptide comprising the amino acid sequence of XXXXXSXXRXRRXXX (SEQ ID NO: 780), more preferably a polypeptide comprising an amino acid sequence in which any 1, 2, 3, 4, 5, 6, 7, 8, 9, 10, 11 or 12 of the amino acids at positions 1-8, 10 and 13-15 of SEQ ID NO: 781 are substituted by alanine, further preferably a polypeptide comprising an amino acid sequence in which any 1, 2, 3, 4, 5, 6, 7, 8, 9, 10 or 11 of the amino acids at positions 1-5, 7, 8, 10 and 13-15 of XXX XXXXEEEXX are substituted by other amino acids;
(19β-4) a polypeptide comprising the amino acid sequence of XXXXXXXXXXEEEXX, preferably a polypeptide comprising the amino acid sequence of XKEEEHQEXXEEEQX (SEQ ID NO: 783), more preferably a polypeptide comprising an amino acid sequence in which any 1, 2, 3, 4, 5, 6, 7, 8, 9, 10, 11 or 12 of the amino acids at positions 1-10, 14 and 15 of SEQ ID NO: 784 are substituted by alanine, further preferably a polypeptide comprising an amino acid sequence in which any 1, 2, 3 or 4 of the amino acids at positions 1, 9, 10 and 15 of SEQ ID NO: 784 are substituted by other amino acids;
(19β-5) a polypeptide comprising the amino acid sequence of XXGKXXXXXXXXXXX, preferably a polypeptide comprising an amino acid sequence in which any 1, 2, 3, 4, 5, 6, 7, 8, 9, 10, 11, 12 or 13 of the amino acids at positions 1, 2 and 5-15 of SEQ ID NO: 786 are substituted by alanine, more preferably a polypeptide comprising an amino acid sequence in which any 1, 2, 3, 4, 5, 6, 7, 8, 9, 10, 11, 12 or 13 of the amino acids at positions 1, 2 and 5-15 of SEQ ID NO: 786 are substituted by other amino acids.

### (20β) Epitope of Seed Biotin-Containing Protein SBP65 (Spot 20α)

In the present invention, the polypeptide (20β) can be a polypeptide comprising at least one amino acid sequence selected from the group consisting of SEQ ID NOs: 788-792.

In one mode, the polypeptide (20) may not comprise the full-length amino acid sequence (SEQ ID NO: 12) of Seed biotin-containing protein SBP65 or a variant or a homolog having 100% or at least 90%, at least 80% or at least 70% identity to the total length of this amino acid sequence.

SEQ ID NOs: 788-792 correspond to amino acids 131-145 (SEQ ID NO: 788), amino acids 251-258 (SEQ ID NO: 789), amino acids 281-295 (SEQ ID NO: 790), amino acids 441-448 (SEQ ID NO: 791), and amino acids 531-545 (SEQ ID NO: 792), respectively, of SEQ ID NO: 12.

### (21β) Epitope of Ribulose Bisphosphate Carboxylase Large Chain (Spot 21α)

In the present invention, the polypeptide (21β) can be a polypeptide comprising at least one amino acid sequence selected from the group consisting of SEQ ID NOs: 793-802.

In one mode, the polypeptide (21β) may not comprise the full-length amino acid sequence (SEQ ID NO: 4) of Ribulose bisphosphate carboxylase large chain or a variant or a homolog having 100% or at least 90%, at least 80% or at least 70% identity to the total length of this amino acid sequence.

SEQ ID NOs: 793-802 correspond to amino acids 31-34 (SEQ ID NO: 793), amino acids 101-108 (SEQ ID NO: 794), amino acids 201-209 (SEQ ID NO: 795), amino acids 221-235 (SEQ ID NO: 796), and amino acids 237-245 (SEQ ID NO: 797), and amino acids 291-295 (SEQ ID NO: 798), amino acids 351-365 (SEQ ID NO: 799), amino acids 411-417 (SEQ ID NO: 800), amino acids 431-445 (SEQ ID NO: 801), and amino acids 461-475 (SEQ ID NO: 802), respectively, of SEQ ID NO: 4.

### (22β) Epitope of Gamma-Glutamyl Hydrolase (Spot 22α)

In the present invention, the polypeptide (22β) can be a polypeptide comprising at least one amino acid sequence selected from the group consisting of SEQ ID NOs: 803-808.

In one mode, the polypeptide (22) may not comprise the full-length amino acid sequence (SEQ ID NO: 6) of Gamma-glutamyl hydrolase or a variant or a homolog having 100% or at least 90%, at least 80% or at least 70% identity to the total length of this amino acid sequence.

SEQ ID NOs: 803-808 correspond to amino acids 61-75 (SEQ ID NO: 803), amino acids 161-175 (SEQ ID NO: 804), amino acids 241-255 (SEQ ID NO: 805), amino acids 261-275 (SEQ ID NO: 806), amino acids 288-294 (SEQ ID NO: 807), and amino acids 301-315 (SEQ ID NO: 808), respectively, of SEQ ID NO: 6.

### (23β) Epitope of Guanine Nucleotide-Binding Protein Subunit Beta-Like Protein (Spot 23α)

In the present invention, the polypeptide (23) can be a polypeptide comprising at least one amino acid sequence selected from the group consisting of SEQ ID NOs: 809-812.

In one mode, the polypeptide (23β) may not comprise the full-length amino acid sequence (SEQ ID NO: 8) of Guanine nucleotide-binding protein subunit beta-like protein or a variant or a homolog having 100% or at least 90%, at least 80% or at least 70% identity to the total length of this amino acid sequence.

SEQ ID NOs: 809-812 correspond to amino acids 41-55 (SEQ ID NO: 809), amino acids 111-125 (SEQ ID NO: 810), amino acids 227-235 (SEQ ID NO: 811), and amino acids 270-278 (SEQ ID NO: 812), respectively, of SEQ ID NO: 8.

### (24β) Epitope of Protein SLE3 (Spot 24α)

In the present invention, the polypeptide (24β) can be a polypeptide comprising the amino acid sequence of SEQ ID NO: 813.

In one mode, the polypeptide (24β) may not comprise the full-length amino acid sequence (SEQ ID NO: 10) of Protein SLE3 or a variant or a homolog having 100% or at least 90%, at least 80% or at least 70% identity to the total length of this amino acid sequence.

SEQ ID NO: 813 corresponds to amino acids 71-85 of SEQ ID NO: 10.

### (25β) Epitope of Succinyl-CoA Ligase [ADP-Forming] Subunit Beta, Mitochondrial (Spot 25α)

In the present invention, the polypeptide (25β) can be a polypeptide comprising at least one amino acid sequence selected from the group consisting of SEQ ID NOs: 814 and 815.

### (26) Epitope of Phosphoglycerate Kinase, Cytosolic (Spot 26α)

In the present invention, the polypeptide (26β) can be any polypeptide selected from the group consisting of (26β-1) and (26β-2) as defined below:
(26β-1) a polypeptide comprising at least one amino acid sequence selected from the group consisting of SEQ ID NOs: 816 and 819; and
(26β-2) a polypeptide comprising the amino acid sequence of FDKXXXX, preferably a polypeptide comprising the amino acid sequence of FDKFXXX (SEQ ID NO: 818), more preferably a polypeptide comprising an amino acid sequence in which any 1, 2, 3 or 4 of the amino acids at positions 4 to 7 of SEQ ID NO: 819 are substituted by alanine, further preferably a polypeptide comprising an amino acid sequence in which any 1, 2 or 3 of the amino acids at positions 5 to 7 of SEQ ID NO: 819 are substituted by other amino acids.

### (27) Epitope of Alcohol Dehydrogenase 1 (Spot 27α)

In the present invention, the polypeptide (27β) can be a polypeptide comprising the amino acid sequence of SEQ ID NO: 820.

### (28β) Epitope of 35 kDa Seed Maturation Protein Isoform X1 (Spot 28α)

In the present invention, the polypeptide (28β) can be any polypeptide selected from the group consisting of (28β-1) to (28β-4) as defined below:
(28β-1) a polypeptide comprising at least one amino acid sequence selected from the group consisting of SEQ ID NOs: 823, 826, 829 and 830-835;
(28β-2) a polypeptide comprising the amino acid sequence of XQXXXXENKP (SEQ ID NO: 821), preferably a polypeptide comprising the amino acid sequence of EQXQXXENKP (SEQ ID NO: 822), more preferably a polypeptide comprising an amino acid sequence in which any 1, 2, 3, 4 or 5 of the amino acids at positions 1 and 3-6 of SEQ ID NO: 823 are substituted by alanine, further preferably a polypeptide comprising an amino acid sequence in which any 1, 2 or 3 of the amino acids at positions 3, 5 and 6 of SEQ ID NO: 823 are substituted by other amino acids;
(28β-3) a polypeptide comprising the amino acid sequence of XKXXXXEKXXXXXXX, preferably a polypeptide comprising the amino acid sequence of XKDXXXEKXXXXXDX (SEQ ID NO: 825), more preferably a polypeptide comprising an amino acid sequence in which any 1, 2, 3, 4, 5, 6, 7, 8, 9, 10, 11 or 12 of the amino acids at positions 1, 3-6 and 9-15 of SEQ ID NO: 826 are substituted by alanine, further preferably a polypeptide comprising an amino acid sequence in which any 1, 2, 3, 4, 5, 6, 7, 8, 9 or 10 of the amino acids at positions 1, 4-6, 9-13 and 15 of SEQ ID NO: 826 are substituted by other amino acids;
(28β-4) a polypeptide comprising the amino acid sequence of XXXDXXREEXXXXRX (SEQ ID NO: 827), preferably a polypeptide comprising the amino acid sequence of XXXDXXREEEEXXRX (SEQ ID NO: 828), more preferably a polypeptide comprising an amino acid sequence in which any 1, 2, 3, 4, 5, 6, 7, 8, 9 or 10 of the amino acids at positions 1-3, 5, 6, 10-13 and 15 of SEQ ID NO: 829 are substituted by alanine, further preferably a polypeptide comprising an amino acid sequence in which any 1, 2, 3, 4, 5, 6, 7 or 8 of the amino acids at positions 1-3, 5, 6, 12, 13 and 15 of SEQ ID NO: 829 are substituted by other amino acids.

### (29β) Epitope of Glyceraldehyde-3-Phosphate Dehydrogenase Isoform X1 (Spot 29α, 30α, 31α, 32α, 33α)

In the present invention, the polypeptide (29β) can be any polypeptide selected from the group consisting of (29β-1) to (29β-2) as defined below:
(28β-1) a polypeptide comprising at least one amino acid sequence selected from the group consisting of SEQ ID NOs: 838 and 839;
(28β-2) a polypeptide comprising the amino acid sequence of XXXXXXXDKP, preferably a polypeptide comprising the amino acid sequence of XXXXXFXDKP (SEQ ID NO: 837), more preferably a polypeptide comprising an amino acid sequence in which any 1, 2, 3, 4, 5, 6 or 7 of the amino acids at positions 1-7 of SEQ ID NO: 838 are substituted by alanine, further preferably a polypeptide comprising an amino acid sequence in which any 1, 2, 3, 4, 5 or 6 of the amino acids at positions 1-5 and 7 of SEQ ID NO: 838 are substituted by other amino acids.

### (34β) Epitope of Bifunctional UDP-Glucose 4-Epimerase and UDP-Xylose 4-Epimerase 1-Like (Spot 34α)

In the present invention, the polypeptide (34β) can be any polypeptide selected from the group consisting of (34β-1) to (34β-2) as defined below:
(34β-1) a polypeptide comprising at least one amino acid sequence selected from the group consisting of SEQ ID NOs: 842 and 843-846;
(34β-2) a polypeptide comprising the amino acid sequence of XXXXXXXXXXXDDXE, preferably a polypeptide comprising the amino acid sequence of LXXXXXXXXXXDDXE (SEQ ID NO: 841), more preferably a polypeptide comprising an amino acid sequence in which any 1, 2, 3, 4, 5, 6, 7, 8, 9, 10, 11 or 12 of the amino acids at positions 1-11 and 14 of SEQ ID NO: 842 are substituted by alanine, further preferably a polypeptide comprising an amino acid sequence in which any 1, 2, 3, 4, 5, 6, 7, 8, 9, 10 or 11 of the amino acids at positions 2-11 and 14 of SEQ ID NO: 842 are substituted by other amino acids.

### (35β) Epitope of Elongation Factor 1-Alpha (Spot 35α)

In the present invention, the polypeptide (35β) can be any polypeptide selected from the group consisting of (35β-1) to (35β-4) as defined below:
(35β-1) a polypeptide comprising at least one amino acid sequence selected from the group consisting of SEQ ID NOs: 849, 850, 853 and 856;
(35β-2) a polypeptide comprising the amino acid sequence of XXXXDKXXXERXXXE (SEQ ID NO: 847), preferably a polypeptide comprising the amino acid sequence of XXXXDKRVXERXXXE (SEQ ID NO: 848), more preferably a polypeptide comprising an amino acid sequence in which any 1, 2, 3, 4, 5, 6, 7, 8, 9 or 10 of the amino acids at positions 1-4, 7-9 and 12-14 of SEQ ID NO: 849 are substituted by alanine, further preferably a polypeptide comprising an amino acid sequence in which any 1, 2, 3, 4, 5, 6, 7 or 8 of the amino acids at positions 1-4, 9 and 12-14 of SEQ ID NO: 849 are substituted by other amino acids;
(35β-3) a polypeptide comprising the amino acid sequence of PISXFEXXX (SEQ ID NO: 851), preferably a polypeptide comprising the amino acid sequence of PISGFEXXX (SEQ ID NO: 852), more preferably a polypeptide comprising an amino acid sequence in which any 1, 2, 3, 4 or 5 of the amino acids at positions 4 and 7-9 of SEQ ID NO: 853 are substituted by alanine, further preferably a polypeptide comprising an amino acid sequence in which any 1, 2 or 3 of the amino acids at positions 7-9 of SEQ ID NO: 853 are substituted by other amino acids;
(35β-4) a polypeptide comprising the amino acid sequence of XXEXLDXXXX, preferably a polypeptide comprising the amino acid sequence of LLEXLDXXXX (SEQ ID NO: 855), more preferably a polypeptide comprising an amino acid sequence in which any 1, 2, 3, 4, 5, 6 or 7 of the amino acids at positions 1, 2, 4 and 7-10 of SEQ ID NO: 856 are substituted by alanine, further preferably a polypeptide comprising an amino acid sequence in which any 1, 2, 3, 4 or 5 of the amino acids at positions 4 and 7-10 of SEQ ID NO: 856 are substituted by other amino acids.

### (36β) Epitope of Seed Maturation Protein PM34 (Spot 36α, 37α)

In the present invention, the polypeptide (36β) can be any polypeptide selected from the group consisting of (36β-1) to (36β-2) as defined below:
(36β-1) a polypeptide comprising at least one amino acid sequence selected from the group consisting of SEQ ID NOs: 857, 858 and 861;
(36β-2) a polypeptide comprising the amino acid sequence of KXXXXQQQ (SEQ ID NO: 859), preferably a polypeptide comprising the amino acid sequence of KXXXQQQQ (SEQ ID NO: 860), more preferably a polypeptide comprising an amino acid sequence in which any 1, 2, 3 or 4 of the amino acids at positions 2-5 of SEQ ID NO: 861 are substituted by alanine, further preferably a polypeptide comprising an amino acid sequence in which any 1, 2 or 3 of the amino acids at positions 2-4 of SEQ ID NO: 861 are substituted by other amino acids.

### (38β) Epitope of Seed Biotin-Containing Protein SBP65-Like Isoform X1 (Spot 38α)

In the present invention, the polypeptide (38β) can be any polypeptide selected from the group consisting of (38β-1) to (38β-13) as defined below:
(38β-1) a polypeptide comprising at least one amino acid sequence selected from the group consisting of SEQ ID NOs: 864, 867, 869, 871, 874, 877, 880, 882, 884, 887, 889 and 892;
(38β-2) a polypeptide comprising the amino acid sequence of RGKXXXX, preferably a polypeptide comprising an amino acid sequence in which any 1, 2, 3 or 4 of the amino acids at positions 4-7 of SEQ ID NO: 864 are substituted by alanine, further preferably a polypeptide comprising an amino acid sequence in which any 1, 2, 3 or 4 of the amino acids at positions 4-7 of SEQ ID NO: 864 are substituted by other amino acids;
(38β-3) a polypeptide comprising the amino acid sequence of XRGKXXXXXX, preferably a polypeptide comprising the amino acid sequence of RRGKXXXXXX (SEQ ID NO: 866), more preferably a polypeptide comprising an amino acid sequence in which any 1, 2, 3, 4, 5, 6 or 7 of the amino acids at positions 1 and 5-10 of SEQ ID NO: 867 are substituted by alanine, further preferably a polypeptide comprising an amino acid sequence in which any 1, 2, 3, 4, 5 or 6 of the amino acids at positions 5-10 of SEQ ID NO: 867 are substituted by other amino acids;
(38β-4) a polypeptide comprising the amino acid sequence of RXKXXXXXXXXXXXE, preferably a polypeptide comprising an amino acid sequence in which any 1, 2, 3, 4, 5, 6, 7, 8, 9, 10, 11 or 12 of the amino acids at positions 2 and 4-14 of SEQ ID NO: 869 are substituted by alanine, further preferably a polypeptide comprising an amino acid sequence in which any 1, 2, 3, 4, 5, 6, 7, 8, 9, 10, 11 or 12 of the amino acids at positions 2 and 4-14 of SEQ ID NO: 869 are substituted by other amino acids;
(38β-5) a polypeptide comprising the amino acid sequence of XXXXXXXRRXXXXXR, preferably a polypeptide comprising an amino acid sequence in which any 1, 2, 3, 4, 5, 6, 7, 8, 9, 10, 11 or 12 of the amino acids at positions 1-7 and 10-14 of SEQ ID NO: 871 are substituted by alanine, further preferably a polypeptide comprising an amino acid sequence in which any 1, 2, 3, 4, 5, 6, 7, 8, 9, 10, 11 or 12 of the amino acids at positions 1-7 and 10-14 of SEQ ID NO: 871 are substituted by other amino acids;
(38β-6) a polypeptide comprising the amino acid sequence of XXXKGRVVXEXXXXX (SEQ ID NO: 872), preferably a polypeptide comprising the amino acid sequence of LPDKGRVVXESEKKE (SEQ ID NO: 873), more preferably a polypeptide comprising an amino acid sequence in which any 1, 2, 3, 4, 5, 6, 7, 8 or 9 of the amino acids at positions 1-3, 9 and 11-15 of SEQ ID NO: 874 are substituted by alanine, further preferably a polypeptide comprising an amino acid sequence in which any 1, 2, 3, 4, 5, 6, 7, 8 or 9 of the amino acids at positions 1-3, 9 and 11-15 of SEQ ID NO: 874 are substituted by other amino acids;
(38β-7) a polypeptide comprising the amino acid sequence of ATKAKDXXREXXXXX (SEQ ID NO: 875), preferably a polypeptide comprising the amino acid sequence of ATKAKDVTREXXXXX (SEQ ID NO: 876), more preferably a polypeptide comprising an amino acid sequence in which any 1, 2, 3, 4, 5, 6 or 7 of the amino acids at positions 7, 8 and 11-15 of SEQ ID NO: 877 are substituted by alanine, further preferably a polypeptide comprising an amino acid sequence in which any 1, 2, 3, 4 or 5 of the amino acids at positions 11-15 of SEQ ID NO: 877 are substituted by other amino acids;
(38β-8) a polypeptide comprising the amino acid sequence of XGXKGQXKKXXXXXX (SEQ ID NO: 878), preferably a polypeptide comprising the amino acid sequence of XGXKGQTKKXXGEEQ (SEQ ID NO: 879), more preferably a polypeptide comprising an amino acid sequence in which any 1, 2, 3, 4, 5, 6, 7, 8 or 9 of the amino acids at positions 1, 3, 7 and 10-15 of SEQ ID NO: 880 are substituted by alanine, further preferably a polypeptide comprising an amino acid sequence in which any 1, 2, 3 or 4 of the amino acids at positions 1, 3, 10 and 11 of SEQ ID NO: 880 are substituted by other amino acids;
(38β-9) a polypeptide comprising the amino acid sequence of RXXXETKEGX (SEQ ID NO: 881), preferably a polypeptide comprising an amino acid sequence in which any 1, 2, 3 or 4 of the amino acids at positions 2-4 and 10 of SEQ ID NO: 882 are substituted by alanine, further preferably a polypeptide comprising an amino acid sequence in which any 1, 2, 3 or 4 of the amino acids at positions 2-4 and 10 of SEQ ID NO: 882 are substituted by other amino acids;
(38β-10) a polypeptide comprising the amino acid sequence of XXXXNKQSSL (SEQ ID NO: 883), preferably a polypeptide comprising an amino acid sequence in which any 1, 2, 3 or 4 of the amino acids at positions 1˜4 of SEQ ID NO: 884 are substituted by alanine, further preferably a polypeptide comprising an amino acid sequence in which any 1, 2, 3 or 4 of the amino acids at positions 1˜4 of SEQ ID NO: 884 are substituted by other amino acids;
(38β-11) a polypeptide comprising the amino acid sequence of XKXVAXXXXXXXXEX (SEQ ID NO: 885), preferably a polypeptide comprising the amino acid sequence of XKXVAEXXXQXASEL (SEQ ID NO: 886), more preferably a polypeptide comprising an amino acid sequence in which any 1, 2, 3, 4, 5, 6, 7, 8, 9, 10 or 11 of the amino acids at positions 1, 3, 6-13 and 15 of SEQ ID NO: 887 are substituted by alanine, further preferably a polypeptide comprising an amino acid sequence in which any 1, 2, 3, 4, 5 or 6 of the amino acids at positions 1, 3, 7-9 and 11 of SEQ ID NO: 887 are substituted by other amino acids;
(38β-12) a polypeptide comprising the amino acid sequence of XXKVEAEXXX (SEQ ID NO: 888), preferably a polypeptide comprising an amino acid sequence in which any 1, 2, 3, 4 or 5 of the amino acids at positions 1, 2 and 8-10 of SEQ ID NO: 889 are substituted by alanine, further preferably a polypeptide comprising an amino acid sequence in which any 1, 2, 3, 4 or 5 of the amino acids at positions 1, 2 and 8-10 of SEQ ID NO: 889 are substituted by other amino acids;
(38β-13) a polypeptide comprising the amino acid sequence of XXXXIAEIAE (SEQ ID NO: 890), preferably a polypeptide comprising the amino acid sequence of XXETIAEIAE (SEQ ID NO: 891), more preferably a polypeptide comprising an amino acid sequence in which any 1, 2, 3 or 4 of the amino acids at positions 1˜4 of SEQ ID NO: 892 are substituted by alanine, further preferably a polypeptide comprising an amino acid sequence in which any 1 or 2 of the amino acids at positions 1 and 2 of SEQ ID NO: 892 are substituted by other amino acids.

### (39β) Epitope of ATP Synthase Subunit O, Mitochondrial-Like (Spot 39α)

In the present invention, the polypeptide (39β) can be any polypeptide selected from the group consisting of (39β-1) to (39β-2) as defined below:
(39β-1) a polypeptide comprising the amino acid sequence of SEQ ID NOs: 895;
(39β-2) a polypeptide comprising the amino acid sequence of XYXXSXKXXXXEKVX (SEQ ID NO: 893), preferably a polypeptide comprising the amino acid sequence of XYIXSXKXXXXEKVX (SEQ ID NO: 894), more preferably a polypeptide comprising an amino acid sequence in which any 1, 2, 3, 4, 5, 6, 7, 8 or 9 of the amino acids at positions 1, 3, 4, 6, 8-11 and 15 of SEQ ID NO: 895 are substituted by alanine, further preferably a polypeptide comprising an amino acid sequence in which any 1, 2, 3, 4, 5, 6, 7 or 8 of the amino acids at positions 1, 4, 6, 8-11 and 15 of SEQ ID NO: 895 are substituted by other amino acids.

### (40β) Epitope of P24 Oleosin Isoform a (Spot 40α, 56α)

In the present invention, the polypeptide (40β) can be any polypeptide selected from the group consisting of (40β-1) to (40β-3) as defined below:
(40β-1) a polypeptide comprising at least one amino acid sequence selected from the group consisting of SEQ ID NOs: 898, 899 and 902;
(40β-2) a polypeptide comprising the amino acid sequence of YEXGV (SEQ ID NO: 896), preferably a polypeptide comprising the amino acid sequence of YEXGV (SEQ ID NO: 897), more preferably a polypeptide comprising an amino acid sequence in which any 1 or 2 of the amino acids at positions 3 and 6 of SEQ ID NO: 898 are substituted by alanine, further preferably a polypeptide comprising an amino acid sequence in which any 1 or 2 of the amino acids at positions 3 and 6 of SEQ ID NO: 898 are substituted by other amino acids;
(40β-3) a polypeptide comprising the amino acid sequence of XXXXXVTAX, preferably a polypeptide comprising the amino acid sequence of VXXXXVTAX (SEQ ID NO: 901), more preferably a polypeptide comprising an amino acid sequence in which any 1, 2, 3, 4, 5 or 6 of the amino acids at positions 1-5 and 9 of SEQ ID NO: 902 are substituted by alanine, further preferably a polypeptide comprising an amino acid sequence in which any 1, 2, 3, 4 or 5 of the amino acids at positions 2-5 and 9 of SEQ ID NO: 902 are substituted by other amino acids.

### (41β) Epitope of Uncharacterized Protein At3g49720-Like Isoform X2 (Spot 41α)

In the present invention, the polypeptide (41β) can be a polypeptide comprising the amino acid sequence of SEQ ID NO: 903.

### (42β) Epitope of Superoxide Dismutase [Mn], Mitochondrial (Spot 42α, 43α)

In the present invention, the polypeptide (42) can be any polypeptide selected from the group consisting of (42β-1) to (42β-2) as defined below:
(42β-1) a polypeptide comprising the amino acid of SEQ ID NOs: 906;
(42β-2) a polypeptide comprising the amino acid sequence of XXWXXXXXXXEKEXX (SEQ ID NO: 904), preferably a polypeptide comprising the amino acid sequence of XXWXXXXEXXEKESX (SEQ ID NO: 905), more preferably a polypeptide comprising an amino acid sequence in which any 1, 2, 3, 4, 5, 6, 7, 8, 9, 10 or 11 of the amino acids at positions 1, 2, 4-10, 14 and 15 of SEQ ID NO: 906 are substituted by alanine, further preferably a polypeptide comprising an amino acid sequence in which any 1, 2, 3, 4, 5, 6, 7, 8 or 9 of the amino acids at positions 1, 2, 4-7, 9, 10 and 15 of SEQ ID NO: 906 are substituted by other amino acids.

### (44) Epitope of Ferritin-2 (Spot 44α)

In the present invention, the polypeptide (44β) can be any polypeptide selected from the group consisting of (44β-1) to (44β-2) as defined below:
(44β-1) a polypeptide comprising the amino acid of SEQ ID NOs: 909;
(44β-2) a polypeptide comprising the amino acid sequence of XXXXEEEREHAXQLI (SEQ ID NO: 907), preferably a polypeptide comprising an amino acid sequence in which any 1, 2, 3, 4 or 5 of the amino acids at positions 1˜4 and 12 of SEQ ID NO: 909 are substituted by alanine, more preferably a polypeptide comprising an amino acid sequence in which any 1, 2, 3, 4 or 5 of the amino acids at positions 1˜4 and 12 of SEQ ID NO: 909 are substituted by other amino acids.

In one mode, the polypeptide (44β) may not comprise the full-length amino acid sequence (SEQ ID NO: 14) of Ferritin-2 or a variant or a homolog having 100% or at least 90%, at least 80% or at least 70% identity to the total length of this amino acid sequence.

SEQ ID NO: 909 corresponds to amino acids 131-145 of SEQ ID NO: 14.

### (45β) Epitope of Uncharacterized Protein LOC100499771 (Spot 45α)

In the present invention, the polypeptide (45β) can be a polypeptide comprising at least one amino acid sequence selected from the group consisting of SEQ ID NOs: 910-912.

### (46β) Epitope of Seed Maturation Protein PM22 (Spot 46α)

In the present invention, the polypeptide (46β) can be any polypeptide selected from the group consisting of (46β-1) to (46β-2) as defined below:
(46β-1) a polypeptide comprising the amino acid of SEQ ID NOs: 915;
(46β-2) a polypeptide comprising the amino acid sequence of XDXDXXXDX, preferably a polypeptide comprising the amino acid sequence of XDIDXXXDX (SEQ ID NO: 914), more preferably a polypeptide comprising an amino acid sequence in which any 1, 2, 3, 4, 5 or 6 of the amino acids at positions 1, 3, 5-7 and 9 of SEQ ID NO: 915 are substituted by alanine, further preferably a polypeptide comprising an amino acid sequence in which any 1, 2, 3 or 4 of the amino acids at positions 1 and 5-7 of SEQ ID NO: 915 are substituted by other amino acids.

### (47β) Epitope of Eukaryotic Translation Initiation Factor 5A-2 (Spot 47α)

In the present invention, the polypeptide (47β) can be a polypeptide comprising at least one amino acid sequence selected from the group consisting of SEQ ID NOs: 916-918.

### (48β) Epitope of Uncharacterized Protein LOC100306570 (Spot 48α)

In the present invention, the polypeptide (48β) can be any polypeptide selected from the group consisting of (48β-1) to (48β-2) as defined below:
(48β-1) a polypeptide comprising at least one amino acid sequence selected from the group consisting of SEQ ID NOs: 921 and 922;
(48β-2) a polypeptide comprising the amino acid sequence of XXXXXXFDK, preferably a polypeptide comprising the amino acid sequence of XXXXPXFDK (SEQ ID NO: 920), more preferably a polypeptide comprising an amino acid sequence in which any 1, 2, 3, 4, 5 or 6 of the amino acids at positions 1-6 of SEQ ID NO: 921 are substituted by alanine, further preferably a polypeptide comprising an amino acid sequence in which any 1, 2, 3, 4 or 5 of the amino acids at positions 1˜4 and 6 of SEQ ID NO: 921 are substituted by other amino acids.

### (49β) Epitope of Bowman-Birk Type Proteinase Inhibitor D-II (Spot 49α)

In the present invention, the polypeptide (49β) can be a polypeptide comprising the amino acid sequence of SEQ ID NO: 923.

In one mode, the polypeptide (49β) may not comprise the full-length amino acid sequence (SEQ ID NO: 2) of Bowman-Birk type proteinase inhibitor D-II or a variant or a homolog having 100% or at least 90%, at least 80% or at least 70% identity to the total length of this amino acid sequence.

SEQ ID NO: 923 corresponds to amino acids 79-83 of SEQ ID NO: 2.

### (50β) Epitope of Uncharacterized Protein LOC100813859 (Spot 50)

In the present invention, the polypeptide (50β) can be any polypeptide selected from the group consisting of (50β-1) to (50β-2) as defined below:
(50β-1) a polypeptide comprising at least one amino acid sequence selected from the group consisting of SEQ ID NOs: 924 and 927;
(50β-2) a polypeptide comprising the amino acid sequence of XXXXKQXXX, preferably a polypeptide comprising the amino acid sequence of XXDXKQXXX, more preferably a polypeptide comprising an amino acid sequence in which any 1, 2, 3, 4, 5, 6 or 7 of the amino acids at positions 1˜4 and 7-9 of SEQ ID NO: 927 are substituted by alanine, further preferably a polypeptide comprising an amino acid sequence in which any 1, 2, 3, 4, 5 or 6 of the amino acids at positions 1, 2, 4 and 7-9 of SEQ ID NO: 927 are substituted by other amino acids.

### (51β) Epitope of 14-3-3-Like Protein (Spot 51α)

In the present invention, the polypeptide (51β) can be a polypeptide comprising at least one amino acid sequence selected from the group consisting of SEQ ID NOs: 928-930.

### (52β) Epitope of Probable Fructokinase-4 (Spot 52α)

In the present invention, the polypeptide (52β) comprising an epitope can be any polypeptide selected from the group consisting of (52β-1) and (52β-2) as defined below:
(52β-1) a polypeptide comprising at least one amino acid sequence selected from the group consisting of SEQ ID NOs: 933 and 934-938; and
(52β-2) a polypeptide comprising the amino acid sequence of XXXXXXSXXEXLXXX, preferably a polypeptide comprising the amino acid sequence of XXGXXXSXXEXLXXX (SEQ ID NO: 932), more preferably a polypeptide comprising an amino acid sequence in which any 1, 2, 3, 4, 5, 6, 7, 8, 9, 10, 11 or 12 of the amino acids at positions 1-6, 8, 9, 11, and 13-15 of SEQ ID NO: 933 are substituted by alanine, further preferably a polypeptide comprising an amino acid sequence in which any 1, 2, 3, 4, 5, 6, 7, 8, 9, 10 or 11 of the amino acids at positions 1, 2, 4-6, 8, 9, 11, and 13-15 of SEQ ID NO: 933 are substituted by other amino acids.

### (53β) Epitope of Triosephosphate Isomerase Isoform X1 (Spot 53α)

In the present invention, the polypeptide (53) can be a polypeptide comprising the amino acid sequence of SEQ ID NO: 939.

### (54β) Epitope of Superoxide Dismutase [Fe], Chloroplastic Isoform X1 (Spot 54α)

In the present invention, the polypeptide (54β) can be any polypeptide selected from the group consisting of (54β-1) to (54β-2) as defined below:
(54β-1) a polypeptide comprising at least one amino acid sequence selected from the group consisting of SEQ ID NOs: 940 and 943;
(54β-2) a polypeptide comprising the amino acid sequence of KXXXXTX, preferably a polypeptide comprising the amino acid sequence of KXXXXTQ, more preferably a polypeptide comprising an amino acid sequence in which any 1, 2, 3, 4 or 5 of the amino acids at positions 2-5 and 7 of SEQ ID NO: 943 are substituted by alanine, further preferably a polypeptide comprising an amino acid sequence in which any 1, 2, 3 or 4 of the amino acids at positions 2-5 of SEQ ID NO: 943 are substituted by other amino acids.

### (55β) Epitope of 51 kDa Seed Maturation Protein Precursor (Spot 55α)

In the present invention, the polypeptide (55β) can be any polypeptide selected from the group consisting of (55β-1) to (55β-4) as defined below:
(55β-1) a polypeptide comprising at least one amino acid sequence selected from the group consisting of SEQ ID NOs: 946, 949 and 952;
(55β-2) a polypeptide comprising the amino acid sequence of TKXXXSDXX (SEQ ID NO: 944), preferably a polypeptide comprising the amino acid sequence of TKDXXSDXX (SEQ ID NO: 945), more preferably a polypeptide comprising an amino acid sequence in which any 1, 2, 3, 4 or 5 of the amino acids at positions 3-5, 8 and 9 of SEQ ID NO: 946 are substituted by alanine, further preferably a polypeptide comprising an amino acid sequence in which any 1, 2, 3 or 4 of the amino acids at positions 4, 5, 8 and 9 of SEQ ID NO: 946 are substituted by other amino acids;
(55β-3) a polypeptide comprising the amino acid sequence of VKDXXXDA (SEQ ID NO: 947), preferably a polypeptide comprising the amino acid sequence of VKDYXXDA (SEQ ID NO: 948), more preferably a polypeptide comprising an amino acid sequence in which any 1, 2 or 3 of the amino acids at positions 4-6 of SEQ ID NO: 949 are substituted by alanine, further preferably a polypeptide comprising an amino acid sequence in which any 1 or 2 of the amino acids at positions 5 and 6 of SEQ ID NO: 949 are substituted by other amino acids;
(55β-4) a polypeptide comprising the amino acid sequence of KDXXXXXXQ, preferably a polypeptide comprising the amino acid sequence of KDXXXXXAQ (SEQ ID NO: 951), more preferably a polypeptide comprising an amino acid sequence in which any 1, 2, 3, 4, 5 or 6 of the amino acids at positions 3-8 of SEQ ID NO: 952 are substituted by alanine, further preferably a polypeptide comprising an amino acid sequence in which any 1, 2, 3, 4 or 5 of the amino acids at positions 3-7 of SEQ ID NO: 952 are substituted by other amino acids.

### (57β) Epitope of Gly m 4

In the present invention, the polypeptide (57β) can be any polypeptide selected from the group consisting of (56β-1) to (56β-2) as defined below:
(57β-1) a polypeptide comprising the amino acid of SEQ ID NOs: 955;
(57β-2) a polypeptide comprising the amino acid sequence of XXXXXXXEXYXLXHP (SEQ ID NO: 953), preferably a polypeptide comprising the amino acid sequence of XXXXXXIEAYLLAHP (SEQ ID NO: 954), more preferably a polypeptide comprising an amino acid sequence in which any 1, 2, 3, 4, 5, 6, 7, 8, 9 or 10 of the amino acids at positions 1-7, 9, 11 and 13 of SEQ ID NO: 955 are substituted by alanine, further preferably a polypeptide comprising an amino acid sequence in which any 1, 2, 3, 4, 5 or 6 of the amino acids at positions 1-6 of SEQ ID NO: 955 are substituted by other amino acids.

In one mode, the polypeptide (57) may not comprise the full-length amino acid sequence (UniProt accession number: P26987.1) of Gly m 4 or a variant or a homolog having 100% or at least 90%, at least 80% or at least 70% identity to the total length of this amino acid sequence.

SEQ ID NO: 955 corresponds to amino acids 141-155 of UniProt accession number: P26987.1.

### (58) Epitopes of Gly m 5α and Gly m 5α′

In the present invention, the polypeptide (58β) can be a polypeptide comprising at least one amino acid sequence selected from the group consisting of SEQ ID NOs: 956-960.

In one mode, the polypeptide (58β) comprising an epitope may not comprise the full-length amino acid sequence (NCBI accession number: NP_001236856.1) of Gly m 5a or the full-length amino acid sequence (SEQ ID NO: NCBI accession number: NP_001237316.1) of Gly m 5α′, or a variant or a homolog having 100% or at least 90%, at least 80% or at least 70% identity to the total length of any of these amino acid sequences.

SEQ ID NOs: 956-960 correspond to amino acids 173-181 (SEQ ID NO: 956), amino acids 294-301 (SEQ ID NO: 957), amino acids 301-309 (SEQ ID NO: 958), amino acids 414-422 (SEQ ID NO: 959), and amino acids 294-301 (SEQ ID NO: 960), respectively, of NCBI accession number: NP_001236856.1.

### (59β) Epitopes of Gly m 6.01 and Gly m 6.02

In the present invention, the polypeptide (59) can be a polypeptide comprising at least one amino acid sequence selected from the group consisting of SEQ ID NOs: 961 and 962.

In one mode, the polypeptide (59β) may not comprise the full-length amino acid sequence (UniProt accession number: CAA33215.1) of Gly m 6.01 or the full-length amino acid sequence (UniProt accession number: BAA00154.1) of Gly m 6.02, or a variant or a homolog having 100% or at least 90%, at least 80% or at least 70% identity to the total length of any of these amino acid sequences.

SEQ ID NOs: 961 and 962 correspond to amino acids 201-215 and amino acids 451-465, respectively, of UniProt accession number: CAA33215.1.

### (60β) Epitope of Gly m 8

In the present invention, the polypeptide (60β) can be a polypeptide comprising the amino acid sequence of SEQ ID NO: 963.

In one mode, the polypeptide (60β) may not comprise the full-length amino acid sequence (UniProt accession number: AAB71140.1) of Gly m 8 or a variant or a homolog having 100% or at least 90%, at least 80% or at least 70% identity to the total length of this amino acid sequence.

SEQ ID NO: 963 corresponds to amino acids 71-85 of UniProt accession number: AAB71140.1.

The residue of X as to the aforementioned polypeptides (1β) to (60β) is an amino acid residue at a site where binding activity against IgE antibodies from allergic patients was maintained even after substitution by alanine in alanine scanning described in Example 4. It is well known to those skilled in the art that even when such a site is substituted by any other amino acids, it is highly probable that this binding activity against IgE antibodies is maintained.

The lengths of the aforementioned polypeptides (1β) to (60β) are not particularly limited. In a preferred mode, the lengths of the aforementioned polypeptides (1β) to (60β) can be at least 500 amino acids, at least 300 amino acids, at least 200 amino acids, at least 100 amino acids, at least 50 amino acids, at least 30 amino acids, at least 20 amino acids, at least 10 amino acids, or at least 5 amino acids.

The aforementioned polypeptides (1β) to (60β) may be prepared by a technique of chemical synthesis such as solid-phase peptide synthesis. Alternatively, polypeptides comprising an epitope may be obtained by expressing them as recombinant proteins using a genetic recombination technique well known to those skilled in the art and by separating and purifying them using protein purification methods well known to those skilled in the art.

### Diagnosis Kit and Method (2)

The present invention provides a method for providing an indicator for diagnosing an allergy in a subject, the method comprising the steps of:
(i) contacting a sample obtained from the subject with an antigen, wherein the sample is a solution comprising an Ig antibody;
(ii) detecting binding between the IgE antibody present in the sample obtained from the subject and the antigen; and
(iii) when the binding between the IgE antibody in the subject and the antigen is detected, an indicator of the fact that the subject is allergic is provided; wherein the antigen is at least one of the aforementioned polypeptides (1β) to (60), or a polypeptide in which two or more of the aforementioned polypeptides (1β) to (60β) are joined together via or without a spacer.

Hereinafter, the at least one of the aforementioned polypeptides (1β) to (60β), or the polypeptide in which two or more of the aforementioned polypeptides (1β) to (60β) are joined together via or without a spacer is referred to as the “antigen including (1β) to (60β)” in the present specification. The type of the spacer is not particularly limited, and an ordinary spacer that is used by those skilled in the art for joining together two or more peptides can be used. The spacer may be, for example, a hydrocarbon chain such as Acp (6)-OH.

The sample obtained from a subject is as described above in the subsection titled “Diagnosis kit and method (1)”.

Detection of contact and binding between the sample obtained from a subject and the antigen can be carried out by using a known method described above in the subsection titled “Diagnosis kit and method (1)”, such as ELISA (Enzyme-Linked Immunosorbent Assay), sandwich immunoassay, immunoblotting, immunoprecipitation, or immunochromatography.

The antigen may be provided as an isolated antigen including (1β) to (60β) in a state immobilized on a carrier. In this case, the steps (i) and (ii) mentioned above can be carried out using ELISA or sandwich immunoassay, or the like. In this respect, the step (i) mentioned above can be carried out by contacting the sample obtained from a subject with an antigen-immobilized surface.

The antigen including (1β) to (60β) specifically binds to IgE antibodies from allergic patients. Therefore, when binding between an IgE antibody from a subject and the antigen is detected, an indicator of the fact that the subject is allergic to soybean is provided.

The present invention further provides a kit for diagnosing an allergy, comprising at least one antigen including (1β) to (60β). The diagnosis kit of this invention may be used in the aforementioned method for providing an indicator for diagnosing an allergy or in a diagnosis method as described later. The diagnosis kit of this invention may comprise not only the at least one antigen including (1β) to (60β), but also an anti-IgE antibody labeled with an enzyme and a chromogenic or luminescent substrate serving as a substrate for the enzyme. Also, a fluorescent-labeled anti-IgE antibody may be used. In the diagnosis kit of this invention, the antigen may be provided in a state immobilized on a carrier. The diagnosis kit of this invention may also be provided together with instructions on the procedure for diagnosis or a package containing said instructions.

The present invention further provides a composition for diagnosing an allergy, comprising at least one antigen including (1β) to (60β). The diagnosis composition of this invention can be used in a diagnosis method as described below. The diagnosis composition of this invention may further comprise a pharmaceutically acceptable carrier and/or additives commonly used with the antigen of this invention depending on the need.

In one mode, the present invention provides a method for diagnosing an allergy in a subject, the method comprising:
(i) contacting a sample obtained from the subject with an antigen;
(ii) detecting binding between an IgE antibody present in the sample obtained from the subject and the antigen; and
(iii) when the binding between the IgE antibody in the subject and the antigen is detected, diagnosing the subject as being allergic to a soybean;
wherein the antigen is at least one of the aforementioned polypeptides (1β) to (60β), or a polypeptide in which two or more of the aforementioned polypeptides (1β) to (60β) are joined together via or without a spacer (i.e., the antigen including (1β) to (60β)). In this method, the steps (i) and (ii) are performed as described above regarding the corresponding steps of the method for providing an indicator for diagnosing an allergy.

In another mode, the present invention provides a method for diagnosing an allergy in a subject, the method comprising administering to the subject at least one antigen including (1β) to (60β). This method may be performed in the form of a skin test characterized by applying the antigen onto the skin. The skin test includes forms of tests described above in the subsection titled “Diagnosis kit and method (1)”. If a skin reaction such as swelling occurs in a skin portion to which the antigen has been applied, the subject of interest is diagnosed as having an allergy. The amount of the antigen to be applied to the skin in such tests can be, for example, not more than 100 μg per dose.

In yet another mode, the present invention provides at least one antigen including (1β) to (60), intended for use as an active ingredient for a load test for allergy diagnosis.

In still another mode, the present invention provides at least one antigen including (1β) to (60β), intended for use in the diagnosis of an allergy.

In still another mode, the present invention provides use of at least one antigen including (1β) to (60β) for the production of a diagnostic agent for an allergy.

In this subsection, the allergy to be diagnosed or detected can be allergies to allergens in which allergen components having the aforementioned polypeptides (1β) to (60β) are present. Thus, detection of the allergy can be detection of not only an allergy to a single allergen, but also allergies including cross-reactivity.

### Pharmaceutical Composition and Treatment Method (2)

The present invention provides a pharmaceutical composition comprising at least one antigen including (1β) to (60β). In one mode, the aforementioned pharmaceutical composition is used for the treatment of an allergy.

The present invention also provides a method for treating an allergy, the method comprising administering at least one antigen including (1β) to (60β) to a patient in need of a treatment for an allergy.

In another mode, the present invention provides at least one antigen including (1β) to (60), intended for use in the treatment for an allergy. In yet another mode, the present invention provides use of at least one antigen including (1β) to (60β) for the production of a therapeutic agent for an allergy.

In still another mode, the at least one antigen including (1β) to (60β) can be used as an active ingredient for hyposensitization therapy for an allergy. Here, the antigen protein to be used in the hyposensitization therapy may be a polypeptide that has been expressed and purified and may be a polypeptide that has been expressed in food such as rice, such as rice-based vaccine expressing pollen allergens.

The administration route, administration dose, frequency and/or period of the pharmaceutical composition of this invention, and other ingredients to be contained in the pharmaceutical composition, and the dosage form can be as described above in the subsection titled “Pharmaceutical composition and treatment method (1)”. In the case of using the antigen including (1β) to (60β), for example, the dose to an adult patient may be a dose of not more than 100 μg per dose.

In this subsection, the allergy to be treated can be allergies to allergens in which allergen components having the aforementioned polypeptides (1β) to (60β) are present. Thus, detection of the allergy can be detection of not only an allergy to a single allergen, but also allergies including cross-reactivity.

### Tester (2)

The present invention provides a tester comprising an antibody for at least one antigen including (1β) to (60β).

The antibody can be prepared by a conventional method. For example, the antibody may be prepared by immunizing a mammal such as rabbit with the aforementioned antigen including (1β) to (60β). The antibody may be an Ig antibody, a polyclonal antibody, a monoclonal antibody, or an antigen-binding fragment thereof (e.g., Fab, F(ab′) 2, Fab′).

Further, in the aforementioned tester, the antibody may be provided in a form bound to a carrier. The type of the carrier is not particularly limited as long as it is usable for detection of binding between an antibody and the antigen including (1β) to (60β). Any given carrier known to those skilled in the art can be used.

In one mode, the aforementioned tester can be used for the purposes described above in the subsection titled “Tester (1)”.

A method for determining the presence or absence of an antigen is as described above in the subsection titled “Tester (1)”.

Another mode of the present invention includes a tester for determining the presence or absence of an antigen of an allergy in an object of interest, the tester comprising a primer appropriate for an epitope. The primer is not limited and may be designed so as to comprise, for example, a portion of the nucleotide sequence of a nucleic acid encoding any of the amino acid sequences defined above in (1β) to (60β), or a complementary strand thereof. Alternatively, the primer may be designed so as to be the nucleotide sequence of a region upstream or downstream of a portion encoding an epitope that is any of the amino acid sequences defined above in (1β) to (60β), in a nucleic acid encoding a protein comprising the epitope, or a complementary strand thereof. Here, the position of the epitope in the full-length sequence of an antigen is as defined above in the subsections titled “Antigen” and “Epitope of antigen”. Particularly, when mRNA is of interest, the tester has a complementary primer of a poly-A tail.

For example, DNA is amplified by PCR (Polymerase Chain Reaction) including RT-PCR using templated DNA or mRNA obtained from a sample and the aforementioned primer, and the presence or absence of a nucleic acid encoding the amino acid sequences defined above in (1β) to (60β) in the sequence of the amplified DNA is determined to determine the presence or absence of the antigen. Amplification methods by PCR can be exemplified by RACE.

### Allergen-Free Food and the Like (2)

The present invention provides a food raw material or an edible processed product in which at least one antigen including (1β) to (60β) is eliminated or reduced.

The method for eliminating or reducing the antigen of the present invention in a food raw material or an edible processed product is not limited. The techniques described above in the subsection titled “Allergen-free food and the like (1)” may be used. Elimination or reduction of at least one antigen including (1β) to (60β) may be achieved by eliminating or reducing the whole antigen or may be achieved by cleaving or removing the polypeptides defined in (1) to (60β) from the antigen protein. The “removal” includes deletion and modification.

The edible processed product in which the antigen of the present invention is eliminated or reduced may be a processed product of the food raw material in which the antigen of the present invention is eliminated or reduced, such as powdered milk obtained with purified amino acids as a raw material. In the case of using an ordinary food raw material, a treatment for removing or reducing the polypeptide of this invention is performed before or after preparation of an edible processed product. The techniques described in the subsection titled “Allergen-free food and the like (1)” may be used as methods for removing or reducing the antigen of the present invention in an edible processed product obtained with an ordinary food raw material.

### Method for Producing Allergen-Free Food (2)

The present invention provides a method for producing an edible processed product in which an antigen is eliminated or reduced, the method comprising the step of confirming that the antigen is eliminated or reduced, in a production process of the processed product, wherein the antigen is at least one of the aforementioned antigen including (1β) to (60β) and antigens (1α) to (55α).

In the production method, elimination or reduction of an antigen means that at least one antigen including (1β) to (60β) is eliminated or reduced, or the polypeptides defined in (1) to (60β) are cleaved or removed from the antigen protein.

A technique of confirming that the antigen is eliminated or reduced in the production process of the edible processed product is not particularly limited, and any technique capable of detecting at least one of the aforementioned antigen including (1) to (60β) and antigens (1α) to (55α) may be used. For example, the presence or absence of the polypeptide or the antigen in the processed product may be confirmed from the binding activity of an antibody for at least one of the aforementioned antigen including (1β) to (60β) and antigens (1α) to (55α) against a sample containing a material resulting from the production process of the processed product. Details of such a method are as described above in the subsection titled “Diagnosis kit and method (1)”. Thus, in the production method, the “IgE antibody from a subject” described above in the subsection titled “Diagnosis kit and method (1)” is replaced with the “antibody for at least one of the aforementioned polypeptides (1β) to (60) and antigens (1α) to (55α)”, and the “antigen” described above in the subsection titled “Diagnosis kit and method (1)” is replaced with the “sample containing a material resulting from the production process of the edible processed product”. The techniques described above in the subsection titled “Diagnosis kit and method (1)” can be used to confirm that the antigen is eliminated or reduced in the production process of the edible processed product.

### EXAMPLES

The following describes examples of the present invention. The technical scope of this invention is not limited by these examples.

### Example 1: Confirmation of a Protein Pattern

Proteins contained in soybean were investigated using a two-dimensional electrophoresis method described below.

### Protein Extraction

Commercially available dry soybeans (several beans) were powdered. 500 μl of water was added to 50 mg of the powder and shaking extraction was conducted using a vortex mixer at 25° C. for 10 minutes. After the shaking extraction, a liquid protein extract was collected.

Thereafter, the precipitation procedure was repeated twice using a 2D-CleanUP Kit (produced by GE). In the first round of precipitation, the collected liquid protein extract was precipitated by adding TCA (trichloroacetic acid) thereto and the precipitated product produced by this procedure (TCA-precipitated product) was collected. In the second round of precipitation, the TCA-precipitated product collected above was further precipitated by adding acetone thereto and the precipitated product (sample) produced by this procedure was collected.

### Preparation of a Sample Solution

Part of the collected sample (50 μg on a protein weight basis) was dissolved in 150 μL of a DeStreak Rehydration Solution (produced by GE), which is a swelling buffer for first-dimensional isoelectric focusing gels, thereby obtaining a sample solution for first-dimensional isoelectric focusing (sample solution for swelling). The constituents of the DeStreak Rehydration Solution are as mentioned below.
7M thiourea
2M urea
4% (w/v) of CHAPS (3-[(3-cholamidopropyl) dimethylammonio]propanesulfonate)
0.5% (v/v) IPG buffer; produced by GE
Moderate amount of BPB (bromophenol blue)
   Penetration of the Sample into First-Dimensional Isoelectric Focusing Gels

First-dimensional isoelectric focusing gel strips (Immobiline Drystrip IPG gels (pH3-10NL); produced by GE) were immersed in 140 μL of the foregoing sample solution for first-dimensional isoelectric focusing (sample solution for swelling) and impregnated with the solution at room temperature overnight.

In this example, an IPGphor electrophoresis system produced by GE was used.

An electrophoresis tray was filled with silicone oil. Filter paper moisten with water was positioned at both ends of the gel strips impregnated with the sample, and the gel strips were set in the electrophoresis tray such that the gel strips were covered with silicone oil. Electrodes were placed on the gel strips with the filter paper intervening therebetween.

The maximum current of the isoelectric focusing system was set to 75 μA per gel strip, and the first-dimensional isoelectric focusing was carried out according to the following voltage program: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

### SDS Equilibration of Isoelectric Focusing Gels

After the aforementioned first-dimensional isoelectric focusing was done, the gel strips were taken out of the isoelectric focusing system, immersed in an equilibration buffer containing a reducing agent, and shaken at room temperature for 15 minutes. The constituents of the equilibration buffer containing the reducing agent are as mentioned below.
100 mM Tris-HCl (pH 8.0)
6M urea
30% (v/v) glycerol
2% (w/v) SDS
1% (w/v) DTT

Next, the equilibration buffer containing the reducing agent was removed, and then the gel strips were immersed in an equilibration buffer containing an alkylating agent and shaken at room temperature for 15 minutes to obtain SDS-equilibrated gels. The constituents of the equilibration buffer containing the alkylating agent are as mentioned below.
100 mM Tris-HCl (pH 8.0)
6M urea
30% (v/v) glycerol
2% (w/v) SDS
2.5% (w/v) iodoacetamide

### Second-Dimensional SDS-PAGE

In this example, the XCell SureLock Mini-Cell electrophoresis system produced by Life Technologies was used. The second-dimensional electrophoresis gels used were NuPAGE 4-12% Bis-Tris Gels produced by Life Technologies. Also, an electrophoresis buffer composed of the following constituents was prepared and used.
50 mM MOPS
50 mM Tris base
0.1% (w/v) SDS
1 mM EDTA

Further, an agarose solution for gel adhesion was used in this example, which was prepared by dissolving 0.5% (w/v) Agarose S (produced by Nippon Gene Co., Ltd.) and a moderate amount of BPB (bromophenol blue) in the electrophoresis buffer.

SDS-PAGE wells were washed well with the electrophoresis buffer, and then the buffer used for the washing was removed. Next, the washed wells were charged with the fully dissolved agarose solution for gel adhesion. Next, the SDS-equilibrated gel strips were immersed in agarose and closely adhered to second-dimensional electrophoresis gels using tweezers. After it was confirmed that agarose was fully fixed with the gels being closely adhered to each other, electrophoresis was performed at a constant voltage of 200 V for about 45 minutes.

### Fluorescent Staining of Gels

The gels were fluorescently stained with SYPRO Ruby (produced by Life Technologies).

First, an airtight container to be used was washed well in advance with 98% (v/v) ethanol. The electrophoresed second-dimensional electrophoresis gel strips were taken out of the SDS-PAGE system, placed onto the washed airtight container, and treated twice by immersion in 50% (v/v) methanol and 7% (v/v) aqueous solution containing acetic acid for 30 minutes. Then, a further immersion treatment was done for 10 minutes, with the solution being replaced by water. Next, the second-dimensional electrophoresis gel strips were immersed in 40 mL of SYPRO Ruby and shaken at room temperature overnight. Thereafter, the SYPRO Ruby was removed, and then the second-dimensional electrophoresis gel strips were washed with water and shaken in 10% (v/v) methanol and 7% (v/v) aqueous solution containing acetic acid for 30 minutes. Further shaking was done for at least 30 minutes, with the solution being replaced by water.

### Analysis

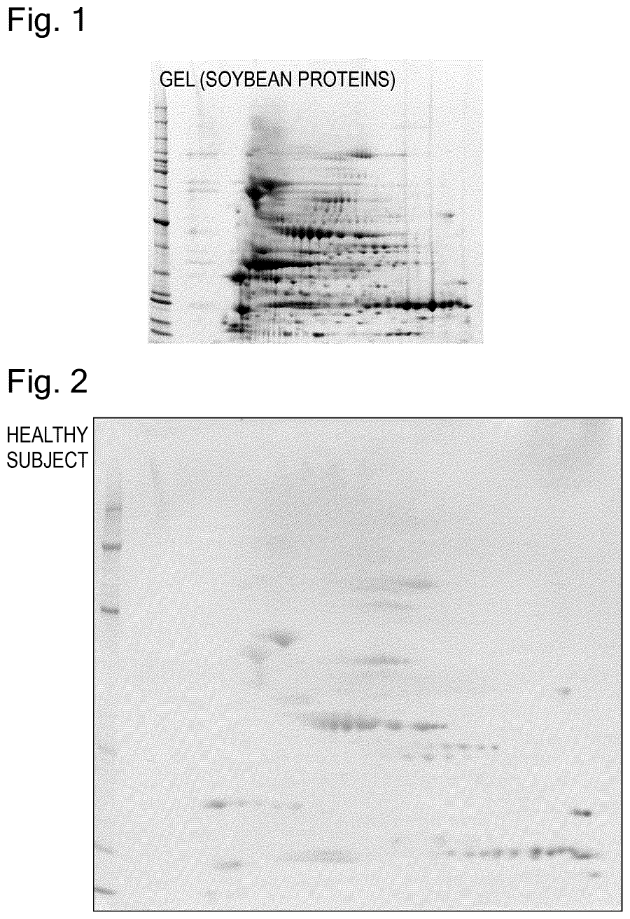

The second-dimensional electrophoresis gels obtained through the foregoing series of treatments were subjected to fluorescent image scanning on Typhoon 9400 (produced by GE). The results of the two-dimensional electrophoresis are shown in FIG. 1. Molecular weight marker bands are found at the left of the panels. The positions of the bands denote particular molecular weights (in KDa).

### Example 2: Identification of Antigens by Immunoblotting

Identification of antigens by immunoblotting was carried out by taking all the steps up to the step of “Second-dimensional SDS-PAGE” as described above in Example 1, followed by the steps of “Transfer to membrane”, “Immunoblotting” and “Analysis” as described below.

### Transfer to Membrane

Transfer to membrane was done using the following transfer system and transfer buffer.
Transfer system: XCell SureLock Mini-Cell and XCell II Blot Module (produced by Life Technologies)
Transfer buffer: NuPAGE Transfer Buffer (X20) (produced by Life Technologies), used in a form diluted 20-fold with milliQ water.

To be specific, proteins in the two-dimensional electrophoresis gels were transferred to a membrane (PVDF membrane) according to the following procedure.
(1) The PVDF membrane was immersed in 100% methanol followed by milliQ water, and then moved into the transfer buffer to hydrophilize the PVDF membrane.
(2) After sponge, filter paper, the gels treated by second-dimensional SDS-PAGE, the hydrophilized PVDF membrane, filter paper, and sponge were put in place in this order, the transfer system was energized at a constant voltage of 30 V for one hour.

### Immunoblotting

Immunoblotting of the membrane was carried out using, as a primary antibody, a serum sample from a patient with a soybean allergy or a serum sample from a healthy subject.

Immunoblotting of the membrane was carried out according to the following procedure.
(1) The transferred membrane was shaken in a 5% skim milk/PBST solution (a PBS buffer containing 0.3% Tween 20 nonionic surfactant) at room temperature for one hour.
(2) The membrane was left to stand in a solution of 3% primary antibody serum in 5% skim milk/PBST at room temperature for one hour.
(3) The membrane was washed with a PBST solution (5 min.×3 times).
(4) The membrane was left to stand in a 1:2500 dilution of the secondary antibody, anti-human IgE-HRP (horseradish peroxidase), with a 3% skim milk/PBST solution at room temperature for one hour.
(5) The membrane was washed with a PBST solution (5 min.×3 times).
(6) The membrane was left to stand in Pierce Western Blotting Substrate Plus (produced by Thermo Fisher Scientific) for 5 minutes.

### Analysis

The membrane obtained through the foregoing series of treatments was subjected to fluorescent image scanning on Typhoon 9400 (produced by GE).

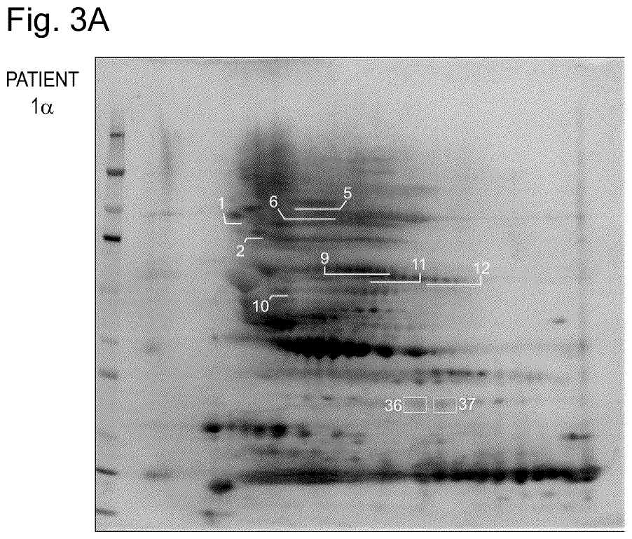

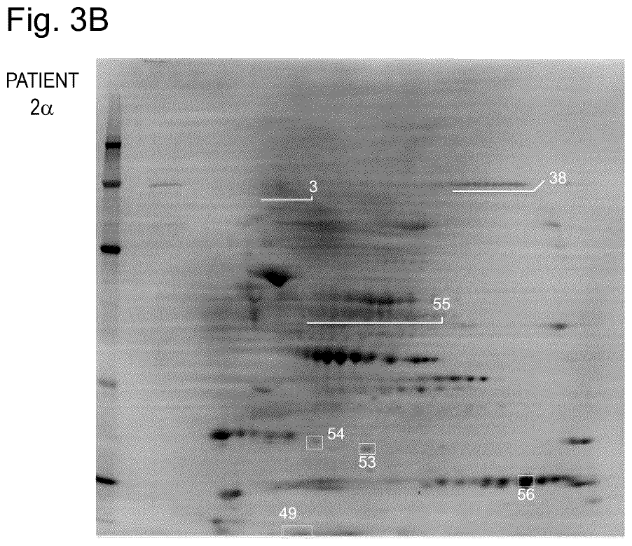

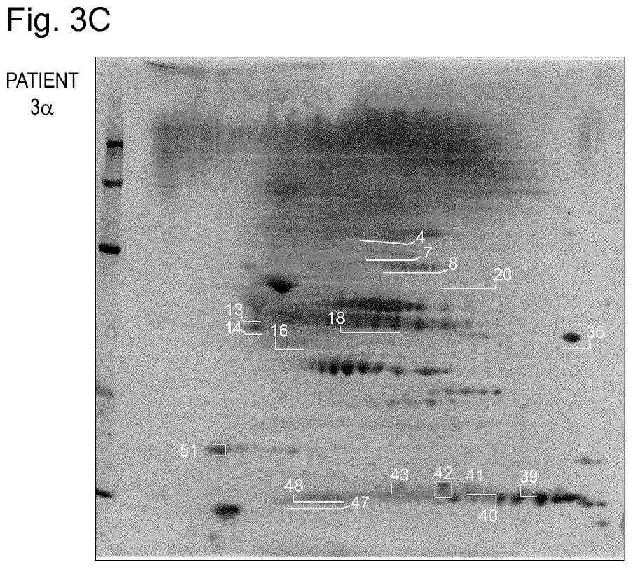

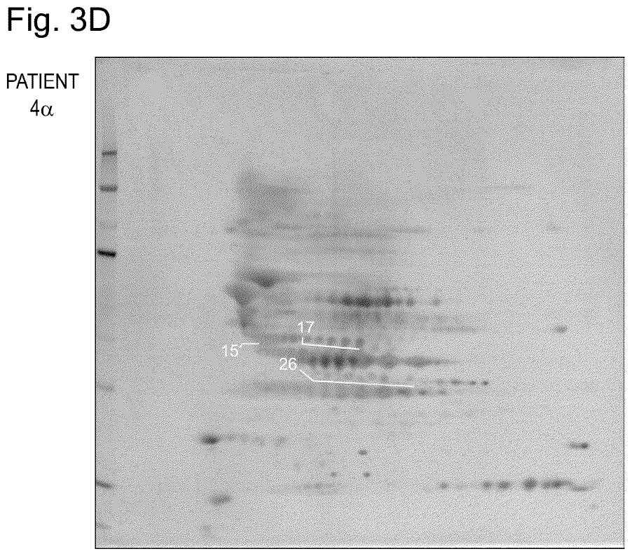

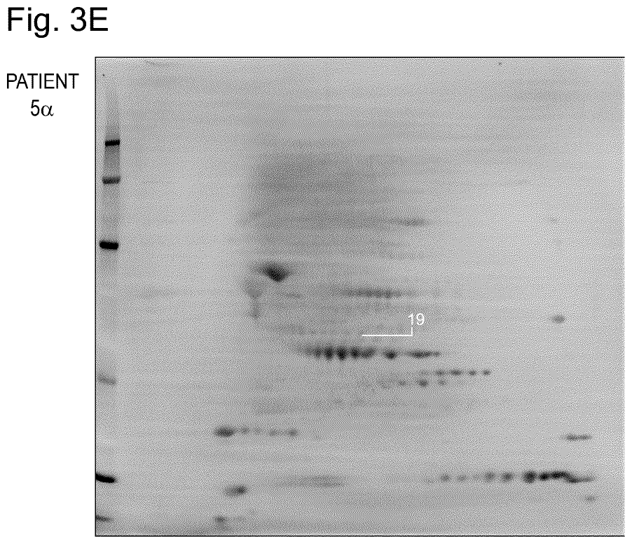

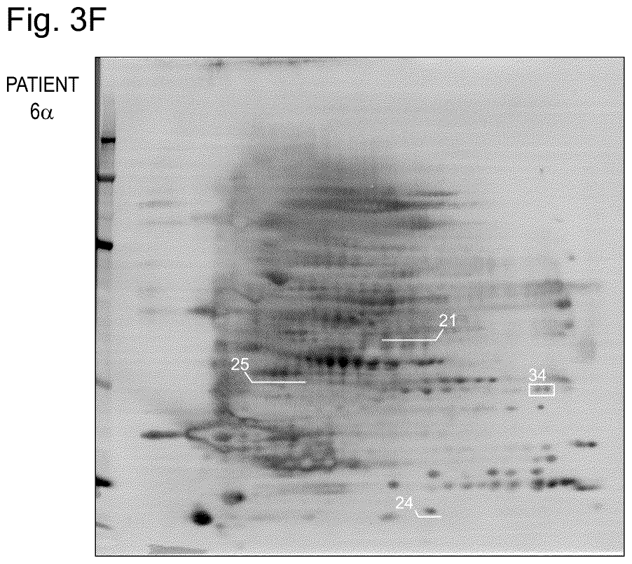

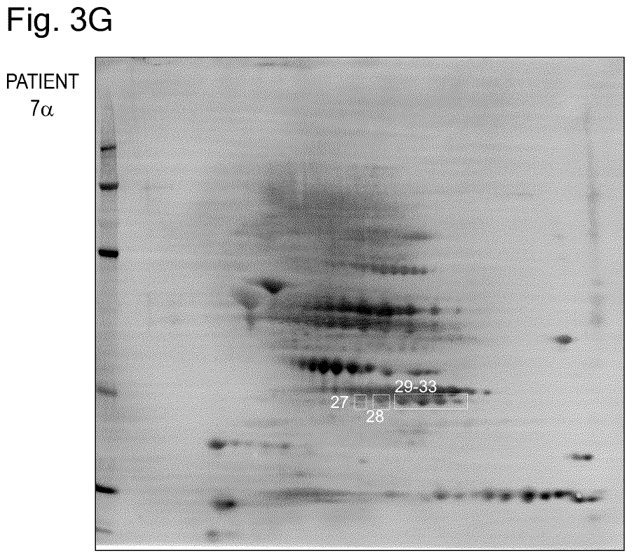

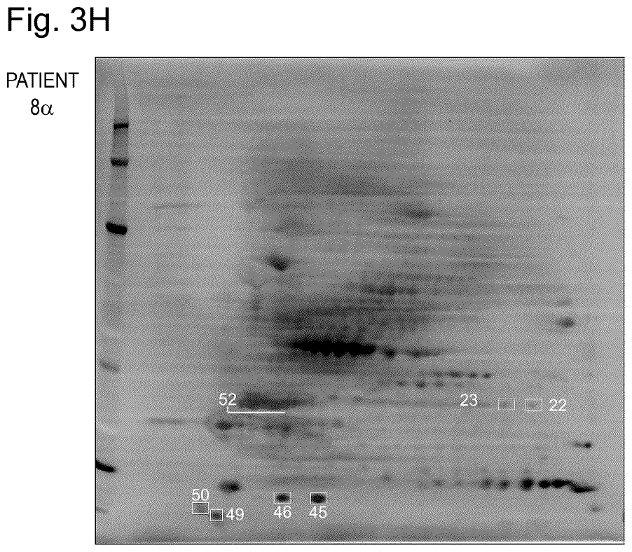

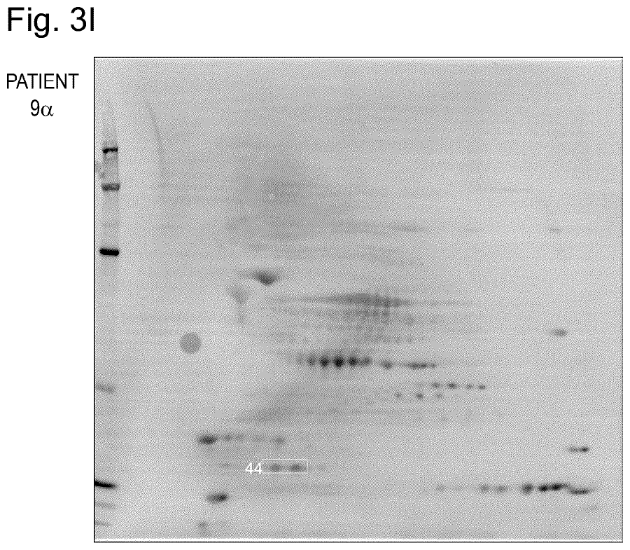

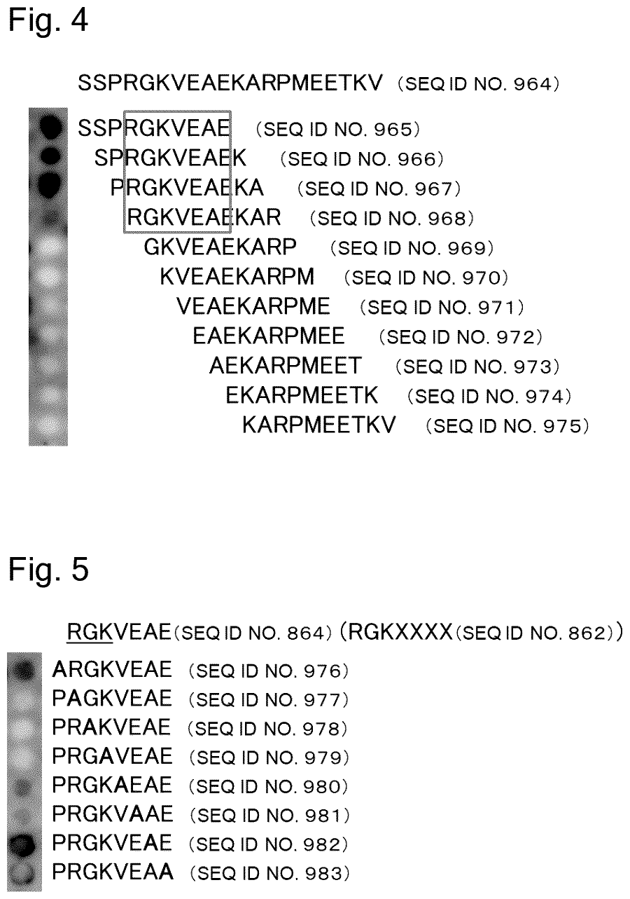

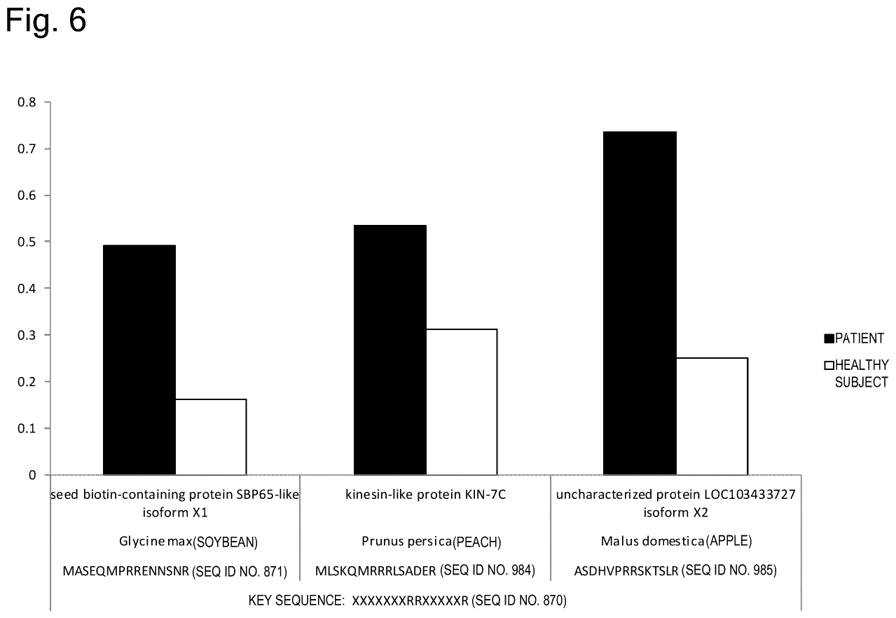

The immunoblots obtained with the serum from the soybean-allergic patient were compared with those obtained with the control serum from the healthy subject. By immunoblotting using the serum from soybean-allergic Patient 1α, Patient 2α, Patient 3α, Patient 4α, Patient 5α, Patient 6α, Patient 7α, Patient 8α, and Patient 9α, 56 spots were detected (FIGS. 3A to I), which are different from the spots detected when the serum of the healthy subject was used (FIG. 2) and different from those of the known soybean allergen proteins.

The molecular weights and isoelectric points of the 56 spots are as follows.
Spot 1α: Molecular weight 80 to 110 kDa, pl 3.0 to 6.0
Spot 2α: Molecular weight 60 to 110 kDa, pl 3.0 to 6.0
Spot 3α: Molecular weight 100 to 150 kDa, pl 4.0 to 6.0
Spot 4α: Molecular weight 80 to 110 kDa, pl 4.0 to 7.0
Spot 5α: Molecular weight 80 to 160 kDa, pl 4.0 to 7.0
Spot 6α: Molecular weight 80 to 110 kDa, pl 4.0 to 7.0
Spot 7α: Molecular weight 60 to 110 kDa, pl 4.0 to 7.0
Spot 8α: Molecular weight 60 to 110 kDa, pl 4.0 to 8.0
Spot 9α: Molecular weight 60 to 110 kDa, pl 4.0 to 8.0
Spot 10α: Molecular weight 40 to 80 kDa, pl 4.0 to 7.0
Spot 11α: Molecular weight 40 to 80 kDa, pl 4.0 to 8.0
Spot 12α: Molecular weight 40 to 80 kDa, pl 4.0 to 8.0
Spot 13α: Molecular weight 40 to 80 kDa, pl 4.0 to 7.0
Spot 14α: Molecular weight 40 to 80 kDa, pl 3.0 to 6.0
Spot 15α, 16α: Molecular weight 30 to 80 kDa, pl 4.0 to 7.0
Spot 17α: Molecular weight 30 to 80 kDa, pl 4.0 to 7.0
Spot 18α: Molecular weight 40 to 80 kDa, pl 4.0 to 8.0
Spot 19α: Molecular weight 40 to 80 kDa, pl 4.0 to 8.0
Spot 20α: Molecular weight 60 to 75 kDa, pl 6.0 to 8.0
Spot 21α: Molecular weight 40 to 60 kDa, pl 5.0 to 8.0
Spot 22α: Molecular weight 25 to 37 kDa, pl 8.0 to 10.0
Spot 23α: Molecular weight 25 to 37 kDa, pl 7.0 to 9.0
Spot 24α: Molecular weight 5 to 20 kDa, pl 5.0 to 7.0
Spot 25α: Molecular weight 30 to 80 kDa, pl 4.0 to 7.0
Spot 26α: Molecular weight 30 to 80 kDa, pl 4.0 to 7.0
Spot 27α: Molecular weight 30 to 80 kDa, pl 4.0 to 7.0
Spot 28α: Molecular weight 20 to 60 kDa, pl 4.0 to 7.0
Spot 29α, 30α, 31α, 32α, 33α: Molecular weight 20 to 60 kDa, pl 4.0 to 8.0
Spot 34α: Molecular weight 20 to 60 kDa, pl 6.0 to 10.0
Spot 35α: Molecular weight 30 to 80 kDa, pl 6.0 to 10.0
Spot 36, 37α: Molecular weight 20 to 60 kDa, pl 4.0 to 8.0
Spot 38α: Molecular weight 80 to 160 kDa, pl 6.0 to 10.0
Spot 39α: Molecular weight 10 to 50 kDa, pl 6.0 to 10.0
Spot 40α, 56α: Molecular weight 10 to 50 kDa, pl 6.0 to 10.0
Spot 41α: Molecular weight 10 to 50 kDa, pl 6.0 to 10.0
Spot 42α, 43α: Molecular weight 10 to 50 kDa, pl 6.0 to 10.0
Spot 44α: Molecular weight 10 to 50 kDa, pl 4.0 to 7.0
Spot 45α: Molecular weight 5 to 40 kDa, pl 4.0 to 7.0
Spot 46α: Molecular weight 5 to 40 kDa, pl 4.0 to 7.0
Spot 47α: Molecular weight 5 to 40 kDa, pl 4.0 to 7.0
Spot 48α: Molecular weight 5 to 40 kDa, pl 4.0 to 8.0
Spot 49α: Molecular weight 5 to 20 kDa, pl 4.0 to 7.0
Spot 50α: Molecular weight 5 to 40 kDa, pl 3.0 to 6.0
Spot 51α: Molecular weight 10 to 50 kDa, pl 3.0 to 6.0
Spot 52α: Molecular weight 20 to 60 kDa, pl 4.0 to 7.0
Spot 53α: Molecular weight 10 to 50 kDa, pl 4.0 to 8.0
Spot 54α: Molecular weight 10 to 50 kDa, pl 4.0 to 8.0
Spot 55α: Molecular weight 40 to 80 kDa, pl 6.0 to 10.0

### Example 3: Mass Spectrometry and Identification of Antigens

The amino acid sequences of the antigens that form the 56 protein spots were identified by mass spectroscopy.

To be specific, protein extraction and mass spectroscopy were done by the following procedure.
(1) Soybeans were subjected to protein extraction, two-dimensional electrophoresis and transfer to membrane by following the procedures described in Example 1 and 2, and the resulting membrane was stained by shaking in a solution of 0.008% Direct blue in 40% ethanol and 10% acetic acid.
(2) Then, the membrane was decolorized by repeating a 5-minute treatment with 40% ethanol and 10% acetic acid three times, washed with water for 5 minutes, and then dried by air.
(3) A protein spot of interest was cut out with a clean cutter blade and put into a centrifugal tube. The cut membrane was subjected to hydrophilization with 50 μL of methanol, followed by washing with 100 μL of water twice and then centrifugal cleaning. Thereafter, 20 μL of 20 mM NH₄HCO₃ and 50% acetonitrile were added.
(4) 1 μL of 1 pmol/μL lysyl endopeptidase (produced by WAKO) was added, and the solution was left to stand at 37° C. for 60 minutes and then collected in a new centrifugal tube. After 20 μL of 20 mM NH₄HCO₃ and 70% acetonitrile were added to the membrane, the membrane was immersed therein at room temperature for 10 minutes, and the resulting solution was further collected. The solution was dissolved with 0.1% formic acid and 10 μL of 4% acetonitrile and transferred to a tube.
(5) The collected solution was dried under reduced pressure, dissolved with 15 μL of solution A (a 0.1% formic acid/4% acetonitrile solution), and analyzed by mass spectroscopy (ESI-TOF5600, produced by AB Sciex).
(6) Identification of proteins based on the MS data obtained with the mass spectrometer was done by searching the NCBI database.

### Results

The mass spectrometry of the spots resulted in the detection of the following amino acid sequences. Furthermore, the spots were identified as the following proteins by the analysis of the mass data obtained from the mass spectrometer for the spots at NCBI.

### Spot 1α:

The amino acid sequences set forth in SEQ ID NOs: 71 to 87 were detected by the mass spectrometry. The protein identified from this mass data was endoplasmin homolog isoform X2 (NCBI accession number XP_006596543.1, amino acid sequence: SEQ ID NO: 70, encoding nucleotide sequence: SEQ ID NO: 69).

The amino acid sequences set forth in SEQ ID NOs: 90 to 104 were detected by the mass spectrometry. The protein identified from this mass data was endoplasmin homolog isoform X2 (NCBI accession number XP_006601356.1, amino acid sequence: SEQ ID NO: 89, encoding nucleotide sequence: SEQ ID NO: 88).

### Spot 2α

The amino acid sequences set forth in SEQ ID NOs: 107 to 120 were detected by the mass spectrometry. The protein identified from this mass data was heat shock cognate protein 80 (NCBI accession number XP_003544594.1, amino acid sequence: SEQ ID NO: 106, encoding nucleotide sequence: SEQ ID NO: 105).

The amino acid sequences set forth in SEQ ID NOs: 123 to 137 were detected by the mass spectrometry. The protein identified from this mass data was heat shock protein 90-1 (NCBI accession number NP_001236612.1, amino acid sequence: SEQ ID NO: 122, encoding nucleotide sequence: SEQ ID NO: 121).

The amino acid sequences set forth in SEQ ID NOs: 140 to 153 were detected by the mass spectrometry. The protein identified from this mass data was hypothetical protein GLYMA_02G302500 (NCBI accession number KRH73936.1, amino acid sequence: SEQ ID NO: 139, encoding nucleotide sequence: SEQ ID NO: 138).

### Spot 3α:

The amino acid sequences set forth in SEQ ID NOs: 62 to 68 were detected by the mass spectrometry. The protein identified from this mass data was Cell division cycle protein 48 homolog (UniProt accession number P54774, amino acid sequence: SEQ ID NO: 16, encoding nucleotide sequence: SEQ ID NO: 15).

### Spot 4α:

The amino acid sequences set forth in SEQ ID NOs: 156, 157 were detected by the mass spectrometry. The protein identified from this mass data was embryo-specific urease isoform X1 (NCBI accession number XP_014630847.1, amino acid sequence: SEQ ID NO: 155, encoding nucleotide sequence: SEQ ID NO: 154).

### Spot 5α:

The amino acid sequences set forth in SEQ ID NOs: 160, 161 were detected by the mass spectrometry. The protein identified from this mass data was alpha-1,4 glucan phosphorylase L isozyme, chloroplastic/amyloplastic (NCBI accession number XP_006603904.1, amino acid sequence: SEQ ID NO: 159, encoding nucleotide sequence: SEQ ID NO: 158).

### Spot 6α:

The amino acid sequences set forth in SEQ ID NOs: 164 to 167 were detected by the mass spectrometry. The protein identified from this mass data was aconitate hydratase 1 (NCBI accession number XP_003537655.1, amino acid sequence: SEQ ID NO: 163, encoding nucleotide sequence: SEQ ID NO: 162).

The amino acid sequences set forth in SEQ ID NOs: 170 to 172 were detected by the mass spectrometry. The protein identified from this mass data was aconitate hydratase 1 (NCBI accession number XP_003517155.1, amino acid sequence: SEQ ID NO: 169, encoding nucleotide sequence: SEQ ID NO: 168).

### Spot 7α:

The amino acid sequences set forth in SEQ ID NOs: 175 to 187 were detected by the mass spectrometry. The protein identified from this mass data was methionine synthase (NCBI accession number NP_001235794.1, amino acid sequence: SEQ ID NO: 174, encoding nucleotide sequence: SEQ ID NO: 173).

### Spot 8α:

The amino acid sequences set forth in SEQ ID NOs: 190 to 199 were detected by the mass spectrometry. The protein identified from this mass data was transketolase, chloroplastic (NCBI accession number XP_003521870.1, amino acid sequence: SEQ ID NO: 189, encoding nucleotide sequence: SEQ ID NO: 188).

### Spot 9α:

The amino acid sequence set forth in SEQ ID NO: 202 was detected by the mass spectrometry. The protein identified from this mass data was lysine-tRNA ligase, cytoplasmic-like isoform X2 (NCBI accession number XP_014625509.1, amino acid sequence: SEQ ID NO: 201, encoding nucleotide sequence: SEQ ID NO: 200).

### Spot 10α

The amino acid sequences set forth in SEQ ID NOs: 205 to 218 were detected by the mass spectrometry. The protein identified from this mass data was chaperonin CPN60-2, mitochondrial (NCBI accession number XP_003556325.1, amino acid sequence: SEQ ID NO: 204, encoding nucleotide sequence: SEQ ID NO: 203).

The amino acid sequences set forth in SEQ ID NOs: 221 to 231 were detected by the mass spectrometry. The protein identified from this mass data was chaperonin CPN60-2, mitochondrial-like isoform X1 (NCBI accession number XP_003535967.1, amino acid sequence: SEQ ID NO: 220, encoding nucleotide sequence: SEQ ID NO: 219).

### Spot 11α:

The amino acid sequences set forth in SEQ ID NOs: 234 to 237 were detected by the mass spectrometry. The protein identified from this mass data was polyadenylate-binding protein 8 isoform X3 (NCBI accession number XP_006578034.1, amino acid sequence: SEQ ID NO: 233, encoding nucleotide sequence: SEQ ID NO: 232).

### Spot 12α:

The amino acid sequence set forth in SEQ ID NO: 240 was detected by the mass spectrometry. The protein identified from this mass data was pyrophosphate-fructose 6-phosphate 1-phosphotransferase subunit alpha-like (NCBI accession number XP_003555655.1, amino acid sequence: SEQ ID NO: 239, encoding nucleotide sequence: SEQ ID NO: 238).

### Spot 13α:

The amino acid sequences set forth in SEQ ID NOs: 243 to 252 were detected by the mass spectrometry. The protein identified from this mass data was protein disulfide isomerase-like protein precursor (NCBI accession number NP_001238009.1, amino acid sequence: SEQ ID NO: 242, encoding nucleotide sequence: SEQ ID NO: 241).

The amino acid sequences set forth in SEQ ID NOs: 255 to 278 were detected by the mass spectrometry. The protein identified from this mass data was protein disulfide-isomerase (NCBI accession number XP_006581588.1, amino acid sequence: SEQ ID NO: 254, encoding nucleotide sequence: SEQ ID NO: 253).

### Spot 14α:

The amino acid sequences set forth in SEQ ID NOs: 281 to 284 were detected by the mass spectrometry. The protein identified from this mass data was V-type proton ATPase subunit B 2 (NCBI accession number XP_003552473.1, amino acid sequence: SEQ ID NO: 280, encoding nucleotide sequence: SEQ ID NO: 279).

The amino acid sequences set forth in SEQ ID NOs: 287 to 289 were detected by the mass spectrometry. The protein identified from this mass data was V-type proton ATPase subunit B 2 (NCBI accession number XP_006605857.1, amino acid sequence: SEQ ID NO: 286, encoding nucleotide sequence: SEQ ID NO: 275).

### Spots 15α, 16α:

The amino acid sequences set forth in SEQ ID NOs: 292 to 304 were detected by the mass spectrometry. The protein identified from this mass data was UTP-glucose-1-phosphate uridylyltransferase (NCBI accession number XP_003544964.1, amino acid sequence: SEQ ID NO: 291, encoding nucleotide sequence: SEQ ID NO: 290).

The amino acid sequences set forth in SEQ ID NOs: 307 to 318 were detected by the mass spectrometry. The protein identified from this mass data was hypothetical protein GLYMA_02G241100 (NCBI accession number KRH72929.1, amino acid sequence: SEQ ID NO: 306, encoding nucleotide sequence: SEQ ID NO: 305).

### Spot 17α:

The amino acid sequences set forth in SEQ ID NOs: 321 to 325 were detected by the mass spectrometry. The protein identified from this mass data was guanosine nucleotide diphosphate dissociation inhibitor 2 (NCBI accession number XP_003531293.1, amino acid sequence: SEQ ID NO: 320, encoding nucleotide sequence: SEQ ID NO: 319).

The amino acid sequences set forth in SEQ ID NOs: 328 to 332 were detected by the mass spectrometry. The protein identified from this mass data was rab GDP dissociation inhibitor alpha-like (NCBI accession number NP_001276273.1, amino acid sequence: SEQ ID NO: 327, encoding nucleotide sequence: SEQ ID NO: 326).

### Spot 18α:

The amino acid sequences set forth in SEQ ID NOs: 335 to 339 were detected by the mass spectrometry. The protein identified from this mass data was hypothetical protein GLYMA_10G028300 (NCBI accession number KRH32039.1, amino acid sequence: SEQ ID NO: 334, encoding nucleotide sequence: SEQ ID NO: 333).

### Spot 19α:

The amino acid sequences set forth in SEQ ID NOs: 342 to 346 were detected by the mass spectrometry. The protein identified from this mass data was sucrose binding protein homolog S-64 (NCBI accession number AAF05723.1, amino acid sequence: SEQ ID NO: 341, encoding nucleotide sequence: SEQ ID NO: 340).

### Spot 20α:

The amino acid sequences set forth in SEQ ID NOs: 35 to 53 were detected by the mass spectrometry. The protein identified from this mass data was Seed biotin-containing protein SBP65 (UniProt accession number Q39846, amino acid sequence: SEQ ID NO: 12, encoding nucleotide sequence: SEQ ID NO: 11).

### Spot 21α:

The amino acid sequences set forth in SEQ ID NOs: 21 to 28 were detected by the mass spectrometry. The protein identified from this mass data was Ribulose bisphosphate carboxylase large chain (UniProt accession number P27066, amino acid sequence: SEQ ID NO: 4, encoding nucleotide sequence: SEQ ID NO: 3).

### Spot 22α:

The amino acid sequences set forth in SEQ ID NOs: 29, 30 were detected by the mass spectrometry. The protein identified from this mass data was Gamma-glutamyl hydrolase (UniProt accession number P93164, amino acid sequence: SEQ ID NO: 6, encoding nucleotide sequence: SEQ ID NO: 5).

### Spot 23α:

The amino acid sequences set forth in SEQ ID NOs: 31, 32 were detected by the mass spectrometry. The protein identified from this mass data was Guanine nucleotide-binding protein subunit beta-like protein (UniProt accession number Q39836, amino acid sequence: SEQ ID NO: 8, encoding nucleotide sequence: SEQ ID NO: 7).

### Spot 24α:

The amino acid sequences set forth in SEQ ID NOs: 33, 34 were detected by the mass spectrometry. The protein identified from this mass data was Protein SLE3 (UniProt accession number C6TOL2, amino acid sequence: SEQ ID NO: 10, encoding nucleotide sequence: SEQ ID NO: 9).

### Spot 25α:

The amino acid sequences set forth in SEQ ID NOs: 349 to 356 were detected by the mass spectrometry. The protein identified from this mass data was succinyl-CoA ligase [ADP-forming] subunit beta, mitochondrial (NCBI accession number XP_003537078.1, amino acid sequence: SEQ ID NO: 348, encoding nucleotide sequence: SEQ ID NO: 347).

The amino acid sequences set forth in SEQ ID NOs: 359 to 365 were detected by the mass spectrometry. The protein identified from this mass data was succinyl-CoA ligase [ADP-forming] subunit beta, mitochondrial (NCBI accession number XP_003542431.1, amino acid sequence: SEQ ID NO: 358, encoding nucleotide sequence: SEQ ID NO: 357).

### Spot 26α:

The amino acid sequences set forth in SEQ ID NOs: 368 to 382 were detected by the mass spectrometry. The protein identified from this mass data was phosphoglycerate kinase, cytosolic (NCBI accession number XP_003546821.1, amino acid sequence: SEQ ID NO: 367, encoding nucleotide sequence: SEQ ID NO: 366).

The amino acid sequences set forth in SEQ ID NOs: 385 to 392 were detected by the mass spectrometry. The protein identified from this mass data was phosphoglycerate kinase, cytosolic (NCBI accession number XP_003531482.1, amino acid sequence: SEQ ID NO: 384, encoding nucleotide sequence: SEQ ID NO: 383).

The amino acid sequences set forth in SEQ ID NOs: 395 to 409 were detected by the mass spectrometry. The protein identified from this mass data was phosphoglycerate kinase, cytosolic (NCBI accession number XP_003531483.1, amino acid sequence: SEQ ID NO: 394, encoding nucleotide sequence: SEQ ID NO: 393).

### Spot 27α:

The amino acid sequences set forth in SEQ ID NOs: 412 to 416 were detected by the mass spectrometry. The protein identified from this mass data was alcohol dehydrogenase 1 (NCBI accession number XP_003526673.1, amino acid sequence: SEQ ID NO: 411, encoding nucleotide sequence: SEQ ID NO: 410).

The amino acid sequences set forth in SEQ ID NOs: 419, 420 were detected by the mass spectrometry. The protein identified from this mass data was alcohol dehydrogenase 1 (NCBI accession number XP_003545664.3, amino acid sequence: SEQ ID NO: 418, encoding nucleotide sequence: SEQ ID NO: 417).

The amino acid sequences set forth in SEQ ID NOs: 423 to 427 were detected by the mass spectrometry. The protein identified from this mass data was alcohol dehydrogenase 1-like (NCBI accession number XP_003544738.1, amino acid sequence: SEQ ID NO: 422, encoding nucleotide sequence: SEQ ID NO: 421).

### Spot 28α:

The amino acid sequences set forth in SEQ ID NOs: 430 to 433 were detected by the mass spectrometry. The protein identified from this mass data was 35 kDa seed maturation protein isoform X1 (NCBI accession number XP_006576267.1, amino acid sequence: SEQ ID NO: 429, encoding nucleotide sequence: SEQ ID NO: 428).

### Spots 29α, 30α, 31α, 32α, 33α:

The amino acid sequences set forth in SEQ ID NOs: 436 to 439 were detected by the mass spectrometry. The protein identified from this mass data was glyceraldehyde-3-phosphate dehydrogenase isoform X1 (NCBI accession number XP_014631598.1, amino acid sequence: SEQ ID NO: 435, encoding nucleotide sequence: SEQ ID NO: 434).

The amino acid sequences set forth in SEQ ID NOs: 442 to 445 were detected by the mass spectrometry. The protein identified from this mass data was glyceraldehyde-3-phosphate dehydrogenase, cytosolic (NCBI accession number XP_006578692.1, amino acid sequence: SEQ ID NO: 441, encoding nucleotide sequence: SEQ ID NO: 440).

The amino acid sequences set forth in SEQ ID NOs: 448 to 451 were detected by the mass spectrometry. The protein identified from this mass data was uncharacterized protein LOC100782924 (NCBI accession number NP_001240046.1, amino acid sequence: SEQ ID NO: 447, encoding nucleotide sequence: SEQ ID NO: 446).

### Spot 34α:

The amino acid sequence set forth in SEQ ID NO: 454 was detected by the mass spectrometry. The protein identified from this mass data was bifunctional UDP-glucose 4-epimerase and UDP-xylose 4-epimerase 1-like (NCBI accession number XP_003532591.1, amino acid sequence: SEQ ID NO: 453, encoding nucleotide sequence: SEQ ID NO: 452).

The amino acid sequences set forth in SEQ ID NOs: 457, 458 were detected by the mass spectrometry. The protein identified from this mass data was uncharacterized protein LOC100803483 (NCBI accession number NP_001241075.1, amino acid sequence: SEQ ID NO: 456, encoding nucleotide sequence: SEQ ID NO: 455).

### Spot 35α:

The amino acid sequences set forth in SEQ ID NOs: 461 to 466 were detected by the mass spectrometry. The protein identified from this mass data was elongation factor 1-alpha (NCBI accession number XP_003553292.1, amino acid sequence: SEQ ID NO: 460, encoding nucleotide sequence: SEQ ID NO: 459).

The amino acid sequences set forth in SEQ ID NOs: 469 to 471 were detected by the mass spectrometry. The protein identified from this mass data was elongation factor 1-alpha (NCBI accession number XP_003547695.1, amino acid sequence: SEQ ID NO: 468, encoding nucleotide sequence: SEQ ID NO: 467).

The amino acid sequences set forth in SEQ ID NOs: 474 to 477 were detected by the mass spectrometry. The protein identified from this mass data was elongation factor-1A isoform X1 (NCBI accession number XP_006600131.1, amino acid sequence: SEQ ID NO: 473, encoding nucleotide sequence: SEQ ID NO: 472).

### Spots 36α, 37α:

The amino acid sequences set forth in SEQ ID NOs: 480 to 483 were detected by the mass spectrometry. The protein identified from this mass data was seed maturation protein PM34 (NCBI accession number NP_001238285.1, amino acid sequence: SEQ ID NO: 479, encoding nucleotide sequence: SEQ ID NO: 478).

The amino acid sequences set forth in SEQ ID NOs: 486, 487 were detected by the mass spectrometry. The protein identified from this mass data was uncharacterized protein LOC100809384 (NCBI accession number NP_001241158.1, amino acid sequence: SEQ ID NO: 485, encoding nucleotide sequence: SEQ ID NO: 484).

The amino acid sequences set forth in SEQ ID NOs: 490 to 493 were detected by the mass spectrometry. The protein identified from this mass data was hypothetical protein GLYMA_10G155300 (NCBI accession number KRH33956.1, amino acid sequence: SEQ ID NO: 489, encoding nucleotide sequence: SEQ ID NO: 488).

### Spot 38α

The amino acid sequences set forth in SEQ ID NOs: 496 to 513 were detected by the mass spectrometry. The protein identified from this mass data was seed biotin-containing protein SBP65-like isoform X1 (NCBI accession number XP_003526237.1, amino acid sequence: SEQ ID NO: 495, encoding nucleotide sequence: SEQ ID NO: 494).

### Spot 39α:

The amino acid sequence set forth in SEQ ID NO: 516 was detected by the mass spectrometry. The protein identified from this mass data was ATP synthase subunit O, mitochondrial-like (NCBI accession number XP_003546884.1, amino acid sequence: SEQ ID NO: 515, encoding nucleotide sequence: SEQ ID NO: 514).

### Spots 40α, 56α:

The amino acid sequences set forth in SEQ ID NOs: 519, 520 were detected by the mass spectrometry. The protein identified from this mass data was P24 oleosin isoform A (NCBI accession number XP_003553822.1, amino acid sequence: SEQ ID NO: 518, encoding nucleotide sequence: SEQ ID NO: 517).

### Spot 41α:

The amino acid sequence set forth in SEQ ID NO: 523 was detected by the mass spectrometry. The protein identified from this mass data was uncharacterized protein At3g49720-like isoform X2 (NCBI accession number XP_006600908.1, amino acid sequence: SEQ ID NO: 522, encoding nucleotide sequence: SEQ ID NO: 521).

### Spots 42α, 43α

The amino acid sequences set forth in SEQ ID NOs: 526 to 531 were detected by the mass spectrometry. The protein identified from this mass data was superoxide dismutase [Mn], mitochondrial (NCBI accession number XP_003526813.1, amino acid sequence: SEQ ID NO: 525, encoding nucleotide sequence: SEQ ID NO: 524).

The amino acid sequences set forth in SEQ ID NOs: 534 to 539 were detected by the mass spectrometry. The protein identified from this mass data was hypothetical protein GLYMA_04G221300 (NCBI accession number KRH64185.1, amino acid sequence: SEQ ID NO: 533, encoding nucleotide sequence: SEQ ID NO: 532).

### Spot 44α:

The amino acid sequences set forth in SEQ ID NOs: 54 to 61 were detected by the mass spectrometry. The protein identified from this mass data was Ferritin-2 (UniProt accession number Q941C4, amino acid sequence: SEQ ID NO: 14, encoding nucleotide sequence: SEQ ID NO: 13).

### Spot 45α:

The amino acid sequences set forth in SEQ ID NOs: 542 to 545 were detected by the mass spectrometry. The protein identified from this mass data was uncharacterized protein LOC100499771 (NCBI accession number NP_001235797.1, amino acid sequence: SEQ ID NO: 541, encoding nucleotide sequence: SEQ ID NO: 540).

The amino acid sequences set forth in SEQ ID NOs: 548 to 551 were detected by the mass spectrometry. The protein identified from this mass data was hypothetical protein GLYMA_09G192800 (NCBI accession number KRH39322.1, amino acid sequence: SEQ ID NO: 547, encoding nucleotide sequence: SEQ ID NO: 546).

### Spot 46α:

The amino acid sequence set forth in SEQ ID NO: 554 was detected by the mass spectrometry. The protein identified from this mass data was seed maturation protein PM22 (NCBI accession number NP_001237935.1, amino acid sequence: SEQ ID NO: 553, encoding nucleotide sequence: SEQ ID NO: 552).

### Spot 47α:

The amino acid sequences set forth in SEQ ID NOs: 557, 558 were detected by the mass spectrometry. The protein identified from this mass data was eukaryotic translation initiation factor 5A-2 (NCBI accession number XP_003549690.1, amino acid sequence: SEQ ID NO: 556, encoding nucleotide sequence: SEQ ID NO: 555).

The amino acid sequences set forth in SEQ ID NOs: 561 to 563 were detected by the mass spectrometry. The protein identified from this mass data was uncharacterized protein LOC100305510 (NCBI accession number NP_001236395.1, amino acid sequence: SEQ ID NO: 560, encoding nucleotide sequence: SEQ ID NO: 559).

### Spot 48α:

The amino acid sequences set forth in SEQ ID NOs: 566 to 570 were detected by the mass spectrometry. The protein identified from this mass data was uncharacterized protein LOC100306570 (NCBI accession number NP_001236895.1, amino acid sequence: SEQ ID NO: 565, encoding nucleotide sequence: SEQ ID NO: 564).

### Spot 49α:

The amino acid sequences set forth in SEQ ID NOs: 17 to 20 were detected by the mass spectrometry. The protein identified from this mass data was Bowman-Birk type proteinase inhibitor D-II (UniProt accession number P01064, amino acid sequence: SEQ ID NO: 2, encoding nucleotide sequence: SEQ ID NO: 1).

### Spot 50α:

The amino acid sequences set forth in SEQ ID NOs: 573 to 576 were detected by the mass spectrometry. The protein identified from this mass data was uncharacterized protein LOC100813859 (NCBI accession number NP_001239816.1, amino acid sequence: SEQ ID NO: 572, encoding nucleotide sequence: SEQ ID NO: 571).

### Spot 51α:

The amino acid sequences set forth in SEQ ID NOs: 579 to 582 were detected by the mass spectrometry. The protein identified from this mass data was 14-3-3-like protein (NCBI accession number XP_003526571.1, amino acid sequence: SEQ ID NO: 578, encoding nucleotide sequence: SEQ ID NO: 577).

### Spot 52α:

The amino acid sequences set forth in SEQ ID NOs: 585 to 588 were detected by the mass spectrometry. The protein identified from this mass data was probable fructokinase-4 (NCBI accession number XP_003543564.1, amino acid sequence: SEQ ID NO: 584, encoding nucleotide sequence: SEQ ID NO: 583).

### Spot 53α:

The amino acid sequences set forth in SEQ ID NOs: 591 to 597 were detected by the mass spectrometry. The protein identified from this mass data was triosephosphate isomerase isoform X1 (NCBI accession number XP_006593480.2, amino acid sequence: SEQ ID NO: 590, encoding nucleotide sequence: SEQ ID NO: 589).

The amino acid sequences set forth in SEQ ID NOs: 600 to 604 were detected by the mass spectrometry. The protein identified from this mass data was triosephosphate isomerase, cytosolic (NCBI accession number XP_003547334.1, amino acid sequence: SEQ ID NO: 599, encoding nucleotide sequence: SEQ ID NO: 598).

### Spot 54α:

The amino acid sequences set forth in SEQ ID NOs: 607 to 611 were detected by the mass spectrometry. The protein identified from this mass data was superoxide dismutase [Fe], chloroplastic isoform X1 (NCBI accession number XP_014627751.1, amino acid sequence: SEQ ID NO: 606, encoding nucleotide sequence: SEQ ID NO: 605).

The amino acid sequences set forth in SEQ ID NOs: 614 to 616 were detected by the mass spectrometry. The protein identified from this mass data was iron-superoxide dismutase (NCBI accession number NP_001237901.1, amino acid sequence: SEQ ID NO: 613, encoding nucleotide sequence: SEQ ID NO: 612).

### Spot 55α:

The amino acid sequences set forth in SEQ ID NOs: 619 to 635 were detected by the mass spectrometry. The protein identified from this mass data was 51 kDa seed maturation protein precursor (NCBI accession number NP_001238138.1, amino acid sequence: SEQ ID NO: 618, encoding nucleotide sequence: SEQ ID NO: 617).

The amino acid sequences set forth in SEQ ID NOs: 638 to 649 were detected by the mass spectrometry. The protein identified from this mass data was Lea protein precursor (NCBI accession number NP_001238201.1, amino acid sequence: SEQ ID NO: 637, encoding nucleotide sequence: SEQ ID NO: 636).

### Example 4: Identification of Epitopes (1)

Identification of epitopes was carried out by the following procedure as to each of the antigen proteins identified in Example 3.

### (A) Epitope Mapping

Epitope mapping was carried out using a library of overlapping peptide fragments (length: 15 amino acids) corresponding to the sequences of proteins bound patient-specifically to IgE antibodies in serum, which were obtained as a result of 2D-western of soybeans.

The peptides to be synthesized were shifted by 10 amino acids. Thus, each peptide had an overlap of 5 amino acids with each of the peptides previous and subsequent thereto.

For preparation of peptide arrays, peptide synthesis of interest was carried out in incremental steps on a modified cellulose membrane by the Intavis SPOT synthesis technique, which is a technique of synthesizing peptides of interest through 9-fluorenylmethoxycarbonyl (Fmoc) chemical reaction, using an automated synthesis apparatus (Intavis MultiPep RS). The aforementioned modified cellulose membrane (hereinafter also referred to as the membrane) bound to the obtained peptides of interest was used in epitope mapping.

The presence or absence of binding, to each peptide fragment, of an IgE antibody present in the serum of a patient allergic to a soybean was measured by immunoblotting using the membrane on which the peptide fragments were synthesized by SPOT. Immunoblotting of the membrane was carried out according to the following procedure using, as a primary antibody, a serum sample from an allergic patient, and anti-human IgE-HRP as a secondary antibody.
(1) The membrane was subjected to hydrophilization with methanol for 5 minutes.
(2) The hydrophilized membrane was shaken overnight in a 5% skim milk/PBST solution (a PBS buffer containing 0.1% Tween 20 nonionic surfactant) at 4° C.
(3) The membrane was washed with a PBST solution three times.
(4) The serum sample (1:40, a 5% skim milk/PBST solution) was added thereto as a primary antibody and shaken overnight at 4° C. In this operation, a protease inhibitor (Complete, produced by Roche) was added.
(5) The membrane was washed with a PBST solution three times.
(6) Anti-human IgE-HRP (1:10,000, a 5% skim milk/PBST solution) was added thereto as a secondary antibody and shaken at room temperature for one hour.
(7) The membrane was washed with a PBST solution three times.
(8) The membrane was left to stand in ECL plus (produced by Thermo Fisher Scientific) for 5 minutes.
(9) The membrane treated as described above in (1) to (8) was subjected to fluorescent image scanning using Amersham Imager 600.

Peptide fragments bound to IgE antibodies from allergic patients in immunoblotting were used as candidate peptides of epitopes.

### (B) Overlapping

Thereafter, for the sequences of peptides bound patient-specifically to IgE antibodies in serum in the epitope mapping described above in (A), a library of overlapping peptide fragments (length: 10 amino acids) was obtained as sequences having the sequences of these peptides as well as additional sequences subsequent to the peptides in the amino acid sequences of antigen proteins on which the epitopes were detected. Synthesis of this library was carried out by the Fmoc chemical synthesis method in the same manner as in (A) mentioned above. The peptides to be synthesized were shifted by one amino acid. Thus, each peptide had an overlap of 9 amino acids with the peptide subsequent thereto.

The presence or absence of binding of an IgE antibody from an allergic patient to each peptide fragment in this library of overlapping peptide fragments was measured by immunoblotting in the same manner as in (A) mentioned above. A common sequence of peptides bound to IgE antibodies from allergic patients was identified to determine peptides having the minimum number of amino acids functioning as an epitope. The peptides determined in (B) are referred to as the “original sequences”.

As one example of the results, the results of determining the original sequences by the overlapping technique as to a partial sequence (a region of amino acids 571-590 of SEQ ID NO: 495/SEQ ID NO: 964 of seed biotin-containing protein SBP65-like isoform X1 (spot 38α) are shown in FIG. 4. Strong binding of polypeptides having the amino acid sequences of SEQ ID NOs: 965-967 to the IgE antibody from the allergic patient was observed. Slightly weak binding of SEQ ID NO: 968 to the IgE antibody from the allergic patient was observed. Binding of SEQ ID NOs: 969-975 to the IgE antibody from the allergic patient was not observed. As a result, SEQ ID NO: 864, a common sequence of SEQ ID NOs: 965-968, was identified as an original sequence.

### (C) Alanine Scanning

From the “original sequences” determined as described above in (B), a library of peptide fragments in which N-terminal amino acids were substituted one by one by alanine according to a technique called alanine scanning (Non Patent Literature 1) was prepared by the Fmoc chemical synthesis method in the same manner as in (A) mentioned above. The presence or absence of binding of an IgE antibody present in the serum of an allergic patient to each peptide fragment was measured by immunoblotting in the same manner as in (A) mentioned above. Amino acids at positions where the binding activity against IgE antibodies from allergic patients was lost by the substitution by alanine were confirmed as positions important for exertion of antigenicity. Also, Amino acids at positions where the binding activity against IgE antibodies from allergic patients was maintained even after the substitution by alanine were confirmed as substitutable positions that were not important for exertion of antigenicity. Common sequences (consensus sequences) important for exertion of antigenicity were found by this analysis.

As one example of the results, the results of performing alanine scanning as to the amino acid sequence of SEQ ID NO: 864 identified in (B) are shown in FIG. 5. Peptides having SEQ ID NOs: 977-979 in which the amino acids at positions 1-3, respectively, of SEQ ID NO: 864 were substituted by alanine lost their binding activity against IgE antibodies from allergic patients. Thus, the amino acids at positions 1-3 of SEQ ID NO: 864 were identified as amino acids important for the binding to IgE antibodies from allergic patients.

### (D) Results

### (1β) Epitope of Endoplasmin Homolog Isoform X2 (Spot 1α)

SEQ ID NOs: 652, 655, 657, and 658-660 were identified as epitopes of endoplasmin homolog isoform X2 by the procedure of (B) mentioned above.

Amino acids important for binding to IgE antibodies from allergic patients were identified by the procedure of (C) mentioned above as to each of SEQ ID NOs: 652, 655, 657, and 658-660.

It was revealed for SEQ ID NO: 652 that the amino acids at positions 3 and 5 are amino acids particularly important for the binding to IgE antibodies from allergic patients, and the amino acid at position 2 is an amino acid that influences the binding to IgE antibodies from allergic patients. The amino acids at positions 1, 4, and 6-8 of SEQ ID NO: 652 had no influence on the binding activity against IgE antibodies from allergic patients when substituted by alanine.

It was revealed for SEQ ID NO: 655 that the amino acids at positions 4-7 are amino acids particularly important for the binding to IgE antibodies from allergic patients, and the amino acid at position 2 is an amino acid that influences the binding to IgE antibodies from allergic patients. The amino acids at positions 1, 3, 8 and 9 of SEQ ID NO: 655 had no influence on the binding activity against IgE antibodies from allergic patients when substituted by alanine.

For SEQ ID NO: 657, the amino acids at positions 1-5 and 11 were amino acids particularly important for the binding to IgE antibodies from allergic patients. The amino acids at positions 6-10 and 12-15 of SEQ ID NO: 657 had no influence on the binding activity against IgE antibodies from allergic patients when substituted by alanine.

For SEQ ID NOs: 658-660, all the amino acids were amino acids particularly important for the binding to IgE antibodies from allergic patients.

### (2β) Epitope of Heat Shock Cognate Protein 80 (Spot 2α)

SEQ ID NOs: 663, 666, 669, 670-672, 675, 676-678, 681, 684, 687, 689, and 690-692 were identified as epitopes of heat shock cognate protein 80 by the procedure of (B) mentioned above.

Amino acids important for binding to IgE antibodies from allergic patients were identified by the procedure of (C) mentioned above as to each of SEQ ID NOs: 663, 666, 669, 670-672, 675-678, 681, 684, 687 and 689-692.

It was revealed for SEQ ID NO: 663 that the amino acids at positions 1, 4 and 5 are amino acids particularly important for the binding to IgE antibodies from allergic patients, and the amino acid at position 7 is an amino acid that influences the binding to IgE antibodies from allergic patients. The amino acids at positions 2, 3, 6, 8 and 9 of SEQ ID NO: 663 had no influence on the binding activity against IgE antibodies from allergic patients when substituted by alanine.

It was revealed for SEQ ID NO: 666 that the amino acids at positions 5-8 are amino acids particularly important for the binding to IgE antibodies from allergic patients, and the amino acid at position 12 is an amino acid that influences the binding to IgE antibodies from allergic patients. The amino acids at positions 1-4, 9-11, and 13-15 of SEQ ID NO: 666 had no influence on the binding activity against IgE antibodies from allergic patients when substituted by alanine.

It was revealed for SEQ ID NO: 669 that the amino acids at positions 2-4 are amino acids particularly important for the binding to IgE antibodies from allergic patients, and the amino acid at position 1 is an amino acid that influences the binding to IgE antibodies from allergic patients. The amino acid at position 5 of SEQ ID NO: 669 had no influence on the binding activity against IgE antibodies from allergic patients when substituted by alanine.

It was revealed for SEQ ID NO: 675 that the amino acids at positions 3-5, 13 and 14 are amino acids particularly important for the binding to IgE antibodies from allergic patients, and the amino acid at position 15 is an amino acid that influences the binding to IgE antibodies from allergic patients. The amino acids at positions 1, 2 and 6-12 of SEQ ID NO: 675 had no influence on the binding activity against IgE antibodies from allergic patients when substituted by alaninc.

It was revealed for SEQ ID NO: 681 that the amino acids at positions 5, 10 and 14 are amino acids particularly important for the binding to IgE antibodies from allergic patients, and the amino acids at positions 6-8 and 15 are amino acids that influence the binding to IgE antibodies from allergic patients. The amino acids at positions 1-4, 9 and 11-13 of SEQ ID NO: 681 had no influence on the binding activity against IgE antibodies from allergic patients when substituted by alanine.

It was revealed for SEQ ID NO: 684 that the amino acids at positions 2, 5, 10, and 11 are amino acids particularly important for the binding to IgE antibodies from allergic patients. The amino acids at positions 1, 3, 4, 6-9, and 12-15 of SEQ ID NO: 684 had no influence on the binding activity against IgE antibodies from allergic patients when substituted by alaninc.

It was revealed for SEQ ID NO: 687 that the amino acids at positions 1, 5, 10, and 11 are amino acids particularly important for the binding to IgE antibodies from allergic patients. The amino acids at positions 2, 3, 4, 6-9 and 12-15 of SEQ ID NO: 687 had no influence on the binding activity against IgE antibodies from allergic patients when substituted by alanine.

It was revealed for SEQ ID NO: 689 that the amino acids at positions 1-3, 12 and 13 are amino acids particularly important for the binding to IgE antibodies from allergic patients. The amino acids at positions 4-11, 14 and 15 of SEQ ID NO: 689 had no influence on the binding activity against IgE antibodies from allergic patients when substituted by alanine.

For SEQ ID NOs: 670-672, 676-678, and 690-692, all the amino acids were amino acids particularly important for the binding to IgE antibodies from allergic patients.

### (3β) Epitope of Cell Division Cycle Protein 48 Homolog (Spot 3α)

SEQ ID NOs: 695, 697, 699, and 700-716 were identified as epitopes of Cell division cycle protein 48 homolog by the procedure of (B) mentioned above.

Amino acids important for binding to IgE antibodies from allergic patients were identified by the procedure of (C) mentioned above as to each of SEQ ID NOs: 695, 697, 699 and 700-716.

It was revealed for SEQ ID NO: 695 that the amino acids at positions 11, 12 and 14 are amino acids particularly important for the binding to IgE antibodies from allergic patients, and the amino acids at positions 4-10, 13 and 15 are amino acids that influence the binding to IgE antibodies from allergic patients. The amino acids at positions 1-3 of SEQ ID NO: 695 had no influence on the binding activity against IgE antibodies from allergic patients when substituted by alanine.

It was revealed for SEQ ID NO: 697 that the amino acids at positions 14 and 15 are amino acids particularly important for the binding to IgE antibodies from allergic patients. The amino acids at positions 1-13 of SEQ ID NO: 697 had no influence on the binding activity against IgE antibodies from allergic patients when substituted by alanine.

It was revealed for SEQ ID NO: 699 that the amino acids at positions 5-7 are amino acids particularly important for the binding to IgE antibodies from allergic patients. The amino acids at positions 1˜4 of SEQ ID NO: 699 had no influence on the binding activity against IgE antibodies from allergic patients when substituted by alanine.

For SEQ ID NOs: 700-716, all the amino acids were amino acids particularly important for the binding to IgE antibodies from allergic patients.

### (4β) Epitope of Embryo-Specific Urease Isoform X1 (Spot 4α)

SEQ ID NOs: 719, 722, 725, 726-728, and 731 were identified as epitopes of embryo-specific urease isoform X1 by the procedure of (B) mentioned above.

Amino acids important for binding to IgE antibodies from allergic patients were identified by the procedure of (C) mentioned above as to each of SEQ ID NOs: 719, 722, 725, 726-728, and 731.

It was revealed for SEQ ID NO: 719 that the amino acids at positions 8-10 are amino acids particularly important for the binding to IgE antibodies from allergic patients, and the amino acid at position 6 is an amino acid that influences the binding to IgE antibodies from allergic patients. The amino acids at positions 1-5 and 7 of SEQ ID NO: 719 had no influence on the binding activity against IgE antibodies from allergic patients when substituted by alanine.

It was revealed for SEQ ID NO: 722 that the amino acids at positions 1 and 4-6 are amino acids particularly important for the binding to IgE antibodies from allergic patients, and the amino acid at position 2 is an amino acid that influences the binding to IgE antibodies from allergic patients. The amino acids at positions 3 and 7 of SEQ ID NO: 722 had no influence on the binding activity against IgE antibodies from allergic patients when substituted by alanine.

It was revealed for SEQ ID NO: 725 that the amino acids at positions 4, 6, 8 and 10 are amino acids particularly important for the binding to IgE antibodies from allergic patients, and the amino acid at position 9 is an amino acid that influences the binding to IgE antibodies from allergic patients. The amino acids at positions 1-3, 5 and 7 of SEQ ID NO: 725 had no influence on the binding activity against IgE antibodies from allergic patients when substituted by alanine.

It was revealed for SEQ ID NO: 731 that the amino acids at positions 4, 6, 10, 11, 14 and 15 are amino acids particularly important for the binding to IgE antibodies from allergic patients, and the amino acid at position 3 is an amino acid that influences the binding to IgE antibodies from allergic patients. The amino acids at positions 1, 2, 5, 7-9, 12 and 13 of SEQ ID NO: 731 had no influence on the binding activity against IgE antibodies from allergic patients when substituted by alanine.

For SEQ ID NOs: 726-728, all the amino acids were amino acids particularly important for the binding to IgE antibodies from allergic patients.

### (5β) Epitope of Alpha-1,4 Glucan Phosphorylase L Isozyme, Chloroplastic/Amyloplastic (Spot 5α)

SEQ ID NOs: 734 and 735-738 were identified as epitopes of alpha-1,4 glucan phosphorylase L isozyme, chloroplastic/amyloplastic by the procedure of (B) mentioned above.

Amino acids important for binding to IgE antibodies from allergic patients were identified by the procedure of (C) mentioned above as to each of SEQ ID NOs: 734, 735-738.

It was revealed for SEQ ID NO: 734 that the amino acids at positions 7, 8, 10, 12 and 14 are amino acids particularly important for the binding to IgE antibodies from allergic patients, and the amino acids at positions 15 is an amino acid that influences the binding to IgE antibodies from allergic patients. The amino acids at positions 1-6, 9, 11 and 13 of SEQ ID NO: 734 had no influence on the binding activity against IgE antibodies from allergic patients when substituted by alanine.

For SEQ ID NOs: 735-738, all the amino acids were amino acids particularly important for the binding to IgE antibodies from allergic patients.

### (6β) Epitope of Aconitate Hydratase 1 (Spot 6α)

SEQ ID NOs: 739 and 740 were identified as epitopes of aconitate hydratase 1 by the procedure of (B) mentioned above.

Amino acids important for binding to IgE antibodies from allergic patients were identified by the procedure of (C) mentioned above as to each of SEQ ID NOs: 739 and 740.

For SEQ ID NOs: 739 and 740, all the amino acids were amino acids particularly important for the binding to IgE antibodies from allergic patients.

### (7β) Epitope of Methionine Synthase (Spot 7α)

SEQ ID NOs: 741 and 744 were identified as epitopes of methionine synthase by the procedure of (B) mentioned above.

Amino acids important for binding to IgE antibodies from allergic patients were identified by the procedure of (C) mentioned above as to each of SEQ ID NOs: 741 and 744.

It was revealed for SEQ ID NO: 744 that the amino acids at positions 8, 9, 11-13 and 15 are amino acids particularly important for the binding to IgE antibodies from allergic patients, and the amino acid at position 10 is an amino acid that influences the binding to IgE antibodies from allergic patients. The amino acids at positions 1-7 and 14 of SEQ ID NO: 744 had no influence on the binding activity against IgE antibodies from allergic patients when substituted by alanine.

For SEQ ID NO: 741, all the amino acids were amino acids particularly important for the binding to IgE antibodies from allergic patients.

(9β) Epitope of Lysine—tRNA Ligase, Cytoplasmic-Like Isoform X2 (Spot 9α)

SEQ ID NOs: 747 and 749 were identified as epitopes of lysine—tRNA ligase, cytoplasmic-like isoform X2 by the procedure of (B) mentioned above.

Amino acids important for binding to IgE antibodies from allergic patients were identified by the procedure of (C) mentioned above as to each of SEQ ID NOs: 747 and 749.

It was revealed for SEQ ID NO: 747 that the amino acids at positions 3, 8 and 9 are amino acids particularly important for the binding to IgE antibodies from allergic patients, and the amino acid at position 1 is an amino acid that influences the binding to IgE antibodies from allergic patients. The amino acids at positions 2, 4-7 and 10 of SEQ ID NO: 747 had no influence on the binding activity against IgE antibodies from allergic patients when substituted by alanine.

It was revealed for SEQ ID NO: 749 that the amino acids at positions 1-5, 7, and 8 are amino acids particularly important for the binding to IgE antibodies from allergic patients. The amino acid at position 6 of SEQ ID NO: 749 had no influence on the binding activity against IgE antibodies from allergic patients when substituted by alanine.

### (11β) Epitope of Polyadenylate-Binding Protein 8 Isoform X3 (Spot 11α)

SEQ ID NOs: 753 and 754 were identified as epitopes of polyadenylate-binding protein 8 isoform X3 by the procedure of (B) mentioned above.

Amino acids important for binding to IgE antibodies from allergic patients were identified by the procedure of (C) mentioned above as to each of SEQ ID NOs: 753 and 754.

It was revealed for SEQ ID NO: 753 that the amino acids at positions 1, 2, 4 and 5 are amino acids particularly important for the binding to IgE antibodies from allergic patients, and the amino acid at position 6 is an amino acid that influences the binding to IgE antibodies from allergic patients. The amino acids at positions 3 and 7-10 of SEQ ID NO: 753 had no influence on the binding activity against IgE antibodies from allergic patients when substituted by alanine.

For SEQ ID NO: 754, all the amino acids were amino acids particularly important for the binding to IgE antibodies from allergic patients.

### (12) Epitope of Pyrophosphate—Fructose 6-Phosphate 1-Phosphotransferase Subunit Alpha-Like (Spot 12α)

SEQ ID NO: 755 was identified as an epitope of pyrophosphate—fructose 6-phosphate 1-phosphotransferase subunit alpha-like by the procedure of (B) mentioned above.

Amino acids important for binding to IgE antibodies from allergic patients were identified by the procedure of (C) mentioned above as to SEQ ID NO: 755.

For SEQ ID NO: 755, all the amino acids were amino acids particularly important for the binding to IgE antibodies from allergic patients.

### (13β) Epitope of Protein Disulfide Isomerase-Like Protein Precursor (Spot 13α)

SEQ ID NO: 758 was identified as an epitope of protein disulfide isomerase-like protein precursor by the procedure of (B) mentioned above.

Amino acids important for binding to IgE antibodies from allergic patients were identified by the procedure of (C) mentioned above as to SEQ ID NO: 758.

It was revealed for SEQ ID NO: 758 that the amino acids at positions 3 and 15 are amino acids particularly important for the binding to IgE antibodies from allergic patients, and the amino acids at positions 5, 10, 12 and 13 are amino acids that influence the binding to IgE antibodies from allergic patients. The amino acids at positions 1, 2, 4, 6-9, 11 and 14 of SEQ ID NO: 758 had no influence on the binding activity against IgE antibodies from allergic patients when substituted by alanine.

### (14β) Epitope of V-Type Proton ATPase Subunit B 2 (Spot 14α)

SEQ ID NOs: 761 and 764 were identified as epitopes of V-type proton ATPase subunit B 2 by the procedure of (B) mentioned above.

Amino acids important for binding to IgE antibodies from allergic patients were identified by the procedure of (C) mentioned above as to each of SEQ ID NOs: 761 and 764.

It was revealed for SEQ ID NO: 761 that the amino acids at positions 6, 7 and 12-15 are amino acids particularly important for the binding to IgE antibodies from allergic patients, and the amino acid at position 8 is an amino acid that influences the binding to IgE antibodies from allergic patients. The amino acids at positions 1-5 and 9-11 of SEQ ID NO: 761 had no influence on the binding activity against IgE antibodies from allergic patients when substituted by alanine.

It was revealed for SEQ ID NO: 764 that the amino acids at positions 1, 2, 12 and 14 are amino acids particularly important for the binding to IgE antibodies from allergic patients, and the amino acids at positions 3-5, 11 and 13 are amino acids that influence the binding to IgE antibodies from allergic patients. The amino acids at positions 6-10 and 15 of SEQ ID NO: 746 had no influence on the binding activity against IgE antibodies from allergic patients when substituted by alanine.

### (15β) Epitope of UTP-Glucose-1-Phosphate Uridylyltransferase (Spots 15α and 16α)

SEQ ID NO: 766 was identified as an epitope of UTP-glucose-1-phosphate uridylyltransferase by the procedure of (B) mentioned above.

Amino acids important for binding to IgE antibodies from allergic patients were identified by the procedure of (C) mentioned above as to SEQ ID NOs: 766.

It was revealed for SEQ ID NO: 766 that the amino acids at positions 2 and 3 are amino acids particularly important for the binding to IgE antibodies from allergic patients. The amino acids at positions 1 and 4-15 of SEQ ID NO: 766 had no influence on the binding activity against IgE antibodies from allergic patients when substituted by alanine.

### (17β) Epitope of Guanosine Nucleotide Diphosphate Dissociation Inhibitor 2 (Spot 17α)

SEQ ID NO: 767 was identified as an epitope of guanosine nucleotide diphosphate dissociation inhibitor 2 by the procedure of (B) mentioned above.

Amino acids important for binding to IgE antibodies from allergic patients were identified by the procedure of (C) mentioned above as to SEQ ID NO: 767.

For SEQ ID NO: 767, all the amino acids were amino acids particularly important for the binding to IgE antibodies from allergic patients.

### (18β) Epitope of Hypothetical Protein GLYMA 10G028300 (Spot 18α)

SEQ ID NOs: 750, 768, 771, 772, and 775 were identified as epitopes of hypothetical protein GLYMA_10G028300 by the procedure of (B) mentioned above.

Amino acids important for binding to IgE antibodies from allergic patients were identified by the procedure of (C) mentioned above as to each of SEQ ID NOs: 750, 768, 771, 772 and 775.

It was revealed for SEQ ID NO: 771 that the amino acids at positions 8 and 11-15 are amino acids particularly important for the binding to IgE antibodies from allergic patients, and the amino acid at position 10 is an amino acid that influences the binding to IgE antibodies from allergic patients. The amino acids at positions 1-7 and 9 of SEQ ID NO: 771 had no influence on the binding activity against IgE antibodies from allergic patients when substituted by alanine.

It was revealed for SEQ ID NO: 775 that the amino acids at positions 2-6 and 8 are amino acids particularly important for the binding to IgE antibodies from allergic patients, and the amino acid at position 7 is an amino acid that influences the binding to IgE antibodies from allergic patients. The amino acids at positions 1, 9 and 10 of SEQ ID NO: 775 had no influence on the binding activity against IgE antibodies from allergic patients when substituted by alanine.

For SEQ ID NOs: 750, 768 and 772, all the amino acids were amino acids particularly important for the binding to IgE antibodies from allergic patients.

### (19β) Epitope of Sucrose Binding Protein Homolog S-64 (Spot 19α)

SEQ ID NOs: 778, 781, 784, 786, and 787 were identified as epitopes of sucrose binding protein homolog S-64 by the procedure of (B) mentioned above.

Amino acids important for binding to IgE antibodies from allergic patients were identified by the procedure of (C) mentioned above as to each of SEQ ID NOs: 778, 781, 784, 786 and 787.

It was revealed for SEQ ID NO: 778 that the amino acids at positions 6-8, 13 and 14 are amino acids particularly important for the binding to IgE antibodies from allergic patients, and the amino acid at position 9 is an amino acid that influences the binding to IgE antibodies from allergic patients. The amino acids at positions 1-5, 10-12 and 15 of SEQ ID NO: 778 had no influence on the binding activity against IgE antibodies from allergic patients when substituted by alanine.

It was revealed for SEQ ID NO: 781 that the amino acids at positions 9, 11 and 12 are amino acids particularly important for the binding to IgE antibodies from allergic patients, and the amino acid at position 6 is an amino acid that influences the binding to IgE antibodies from allergic patients. The amino acids at positions 1-5, 7, 8, 10 and 13-15 of SEQ ID NO: 781 had no influence on the binding activity against IgE antibodies from allergic patients when substituted by alanine.

It was revealed for SEQ ID NO: 784 that the amino acids at positions 11-13 are amino acids particularly important for the binding to IgE antibodies from allergic patients, and the amino acids at positions 2-8 and 14 are amino acids that influence the binding to IgE antibodies from allergic patients. The amino acids at positions 1, 9, 10 and 15 of SEQ ID NO: 784 had no influence on the binding activity against IgE antibodies from allergic patients when substituted by alaninc.

It was revealed for SEQ ID NO: 786 that the amino acids at positions 2 and 3 are amino acids particularly important for the binding to IgE antibodies from allergic patients. The amino acids at positions 1-2 and 5-15 of SEQ ID NO: 786 had no influence on the binding activity against IgE antibodies from allergic patients when substituted by alanine.

For SEQ ID NO: 787, all the amino acids were amino acids particularly important for the binding to IgE antibodies from allergic patients.

### (20β) Epitope of Seed Biotin-Containing Protein SBP65 (Spot 20α)

SEQ ID NOs: 788-792 were identified as epitopes of Seed biotin-containing protein SBP65 by the procedure of (B) mentioned above.

Amino acids important for binding to IgE antibodies from allergic patients were identified by the procedure of (C) mentioned above as to each of SEQ ID NOs: 788-792.

For SEQ ID NOs: 788-792, all the amino acids were amino acids particularly important for the binding to IgE antibodies from allergic patients.

### (21β) Epitope of Ribulose Bisphosphate Carboxylase Large Chain (Spot 21α)

SEQ ID NOs: 793-802 were identified as epitopes of Ribulose bisphosphate carboxylase large chain by the procedure of (B) mentioned above.

Amino acids important for binding to IgE antibodies from allergic patients were identified by the procedure of (C) mentioned above as to each of SEQ ID NOs: 793-802.

For SEQ ID NOs: 793-802, all the amino acids were amino acids particularly important for the binding to IgE antibodies from allergic patients.

### (22β) Epitope of Gamma-Glutamyl Hydrolase (Spot 22α)

SEQ ID NOs: 803-808 were identified as epitopes of Gamma-glutamyl hydrolase by the procedure of (B) mentioned above.

Amino acids important for binding to IgE antibodies from allergic patients were identified by the procedure of (C) mentioned above as to each of SEQ ID NOs: 803-808.

For SEQ ID NOs: 803-808, all the amino acids were amino acids particularly important for the binding to IgE antibodies from allergic patients.

### (23β) Epitope of Guanine Nucleotide-Binding Protein Subunit Beta-Like Protein (Spot 23α)

SEQ ID NOs: 809-812 were identified as epitopes of Guanine nucleotide-binding protein subunit beta-like protein by the procedure of (B) mentioned above.

Amino acids important for binding to IgE antibodies from allergic patients were identified by the procedure of (C) mentioned above as to each of SEQ ID NOs: 809-812.

For SEQ ID NOs: 809-812, all the amino acids were amino acids particularly important for the binding to IgE antibodies from allergic patients.

### (24β) Epitope of Protein SLE3 (Spot 24α)

SEQ ID NO: 813 was identified as an epitope of Protein SLE3 by the procedure of (B) mentioned above.

Amino acids important for binding to IgE antibodies from allergic patients were identified by the procedure of (C) mentioned above as to SEQ ID NO: 813.

For SEQ ID NO: 813, all the amino acids were amino acids particularly important for the binding to IgE antibodies from allergic patients.

### (25) Epitope of Succinyl-CoA Ligase [ADP-Forming] Subunit Beta, Mitochondrial (Spot 25α)

SEQ ID NOs: 814 and 815 were identified as epitopes of succinyl-CoA ligase [ADP-forming] subunit beta, mitochondrial by the procedure of (B) mentioned above.

Amino acids important for binding to IgE antibodies from allergic patients were identified by the procedure of (C) mentioned above as to each of SEQ ID NOs: 814 and 815.

For SEQ ID NOs: 814 and 815, all the amino acids were amino acids particularly important for the binding to IgE antibodies from allergic patients.

### (26β) Epitope of Phosphoglycerate Kinase, Cytosolic (Spot 26α)

SEQ ID NOs: 816 and 819 were identified as epitopes of phosphoglycerate kinase, cytosolic by the procedure of (B) mentioned above.

Amino acids important for binding to IgE antibodies from allergic patients were identified by the procedure of (C) mentioned above as to each of SEQ ID NOs: 816 and 819.

It was revealed for SEQ ID NO: 819 that the amino acids at positions 1-3 are amino acids particularly important for the binding to IgE antibodies from allergic patients, and the amino acid at position 4 is an amino acid that influences the binding to IgE antibodies from allergic patients. The amino acids at positions 5-7 of SEQ ID NO: 819 had no influence on the binding activity against IgE antibodies from allergic patients when substituted by alanine.

For SEQ ID NO: 816, all the amino acids were amino acids particularly important for the binding to IgE antibodies from allergic patients.

### (27) Epitope of Alcohol Dehydrogenase 1 (Spot 27α)

SEQ ID NO: 820 was identified as an epitope of alcohol dehydrogenase 1 by the procedure of (B) mentioned above.

Amino acids important for binding to IgE antibodies from allergic patients were identified by the procedure of (C) mentioned above as to SEQ ID NO: 820.

For SEQ ID NO: 820, all the amino acids were amino acids particularly important for the binding to IgE antibodies from allergic patients.

### (28β) Epitope of 35 kDa Seed Maturation Protein Isoform X1 (Spot 28α)

SEQ ID NOs: 823, 826, 829, and 830-835 were identified as epitopes of 35 kDa seed maturation protein isoform X1 by the procedure of (B) mentioned above.

Amino acids important for binding to IgE antibodies from allergic patients were identified by the procedure of (C) mentioned above as to each of SEQ ID NOs: 823, 826, 829 and 830-835.

It was revealed for SEQ ID NO: 823 that the amino acids at positions 2 and 7-10 are amino acids particularly important for the binding to IgE antibodies from allergic patients, and the amino acids at positions 1, 4 are amino acids that influence the binding to IgE antibodies from allergic patients. The amino acids at positions 3, 5 and 6 of SEQ ID NO: 823 had no influence on the binding activity against IgE antibodies from allergic patients when substituted by alanine.

It was revealed for SEQ ID NO: 826 that the amino acids at positions 2, 7 and 8 are amino acids particularly important for the binding to IgE antibodies from allergic patients, and the amino acids at positions 3 and 14 are amino acids that influence the binding to IgE antibodies from allergic patients. The amino acids at positions 1, 4-6, 9-13 and 15 of SEQ ID NO: 826 had no influence on the binding activity against IgE antibodies from allergic patients when substituted by alanine.

It was revealed for SEQ ID NO: 829 that the amino acids at positions 4, 7-9 and 14 are amino acids particularly important for the binding to IgE antibodies from allergic patients, and the amino acids at positions 10 and 11 are amino acids that influence the binding to IgE antibodies from allergic patients. The amino acids at positions 1-3, 5, 6, 12, 13 and 15 of SEQ ID NO: 829 had no influence on the binding activity against IgE antibodies from allergic patients when substituted by alanine.

For SEQ ID NOs: 830-835, all the amino acids were amino acids particularly important for the binding to IgE antibodies from allergic patients.

### (29β) Epitope of Glyceraldehyde-3-Phosphate Dehydrogenase Isoform X1 (Spots 29α, 30α, 31α, 32α, and 33α)

SEQ ID NOs: 838 and 839 were identified as epitopes of glyceraldehyde-3-phosphate dehydrogenase isoform X1 by the procedure of (B) mentioned above.

Amino acids important for binding to IgE antibodies from allergic patients were identified by the procedure of (C) mentioned above as to each of SEQ ID NOs: 838 and 839.

It was revealed for SEQ ID NO: 838 that the amino acids at positions 8-10 are amino acids particularly important for the binding to IgE antibodies from allergic patients, and the amino acid at position 6 is an amino acid that influences the binding to IgE antibodies from allergic patients. The amino acids at positions 1-5 and 7 of SEQ ID NO: 838 had no influence on the binding activity against IgE antibodies from allergic patients when substituted by alanine.

For SEQ ID NO: 839, all the amino acids were amino acids particularly important for the binding to IgE antibodies from allergic patients.

### (34β) Epitope of Bifunctional UDP-Glucose 4-Epimerase and UDP-Xylose 4-Epimerase 1-Like (Spot 34α)

SEQ ID NOs: 842 and 843-846 were identified as epitopes of bifunctional UDP-glucose 4-epimerase and UDP-xylose 4-epimerase 1-like by the procedure of (B) mentioned above.

Amino acids important for binding to IgE antibodies from allergic patients were identified by the procedure of (C) mentioned above as to each of SEQ ID NOs: 842 and 843-846.

It was revealed for SEQ ID NO: 842 that the amino acids at positions 12, 13 and 15 are amino acids particularly important for the binding to IgE antibodies from allergic patients, and the amino acid at position 1 is an amino acid that influences the binding to IgE antibodies from allergic patients. The amino acids at positions 2-11 and 14 of SEQ ID NO: 842 had no influence on the binding activity against IgE antibodies from allergic patients when substituted by alanine.

For SEQ ID NOs: 843-846, all the amino acids were amino acids particularly important for the binding to IgE antibodies from allergic patients.

### (35β) Epitope of Elongation Factor 1-Alpha (Spot 35α)

SEQ ID NOs: 849, 850, 853, and 856 were identified as epitopes of elongation factor 1-alpha by the procedure of (B) mentioned above.

Amino acids important for binding to IgE antibodies from allergic patients were identified by the procedure of (C) mentioned above as to each of SEQ ID NOs: 849, 850, 853 and 856.

It was revealed for SEQ ID NO: 849 that the amino acids at positions 5, 6, 10, 11 and 15 are amino acids particularly important for the binding to IgE antibodies from allergic patients, and the amino acids at positions 7 and 8 are amino acids that influence the binding to IgE antibodies from allergic patients. The amino acids at positions 1-4, 9 and 12-14 of SEQ ID NO: 849 had no influence on the binding activity against IgE antibodies from allergic patients when substituted by alanine.

It was revealed for SEQ ID NO: 853 that the amino acids at positions 1-3, 5 and 6 are amino acids particularly important for the binding to IgE antibodies from allergic patients, and the amino acid at position 4 is an amino acid that influences the binding to IgE antibodies from allergic patients. The amino acids at positions 7-9 of SEQ ID NO: 853 had no influence on the binding activity against IgE antibodies from allergic patients when substituted by alanine.

It was revealed for SEQ ID NO: 856 that the amino acids at positions 3, 5 and 6 are amino acids particularly important for the binding to IgE antibodies from allergic patients, and the amino acids at positions 1 and 2 are amino acids that influence the binding to IgE antibodies from allergic patients. The amino acids at positions 4 and 7-10 of SEQ ID NO: 856 had no influence on the binding activity against IgE antibodies from allergic patients when substituted by alanine.

For SEQ ID NO: 850, all the amino acids were amino acids particularly important for the binding to IgE antibodies from allergic patients.

### (36β) Epitope of Seed Maturation Protein PM34 (Spots 36α and 37α)

SEQ ID NOs: 857, 858, and 861 were identified as epitopes of seed maturation protein PM34 by the procedure of (B) mentioned above.

Amino acids important for binding to IgE antibodies from allergic patients were identified by the procedure of (C) mentioned above as to each of SEQ ID NOs: 857, 858, and 861.

It was revealed for SEQ ID NO: 861 that the amino acids at positions 1 and 6-8 are amino acids particularly important for the binding to IgE antibodies from allergic patients, and the amino acid at position 5 is an amino acid that influences the binding to IgE antibodies from allergic patients. The amino acids at positions 2-4 of SEQ ID NO: 861 had no influence on the binding activity against IgE antibodies from allergic patients when substituted by alanine.

For SEQ ID NOs: 857 and 858, all the amino acids were amino acids particularly important for the binding to IgE antibodies from allergic patients.

### (38β) Epitope of Seed Biotin-Containing Protein SBP65-Like Isoform X1 (Spot 38α)

SEQ ID NOs: 864, 867, 869, 871, 874, 877, 880, 882, 884, 887, 889, and 892 were identified as epitopes of seed biotin-containing protein SBP65-like isoform X1 by the procedure of (B) mentioned above.

Amino acids important for binding to IgE antibodies from allergic patients were identified by the procedure of (C) mentioned above as to each of SEQ ID NOs: 864, 867, 869, 871, 874, 877, 880, 882, 884, 887, 889, and 892.

It was revealed for SEQ ID NO: 864 that the amino acids at positions 1-3 are amino acids particularly important for the binding to IgE antibodies from allergic patients. The amino acids at positions 4-7 of SEQ ID NO: 864 had no influence on the binding activity against IgE antibodies from allergic patients when substituted by alanine.

It was revealed for SEQ ID NO: 867 that the amino acids at positions 2-4 are amino acids particularly important for the binding to IgE antibodies from allergic patients, and the amino acid at position 1 is an amino acid that influences the binding to IgE antibodies from allergic patients. The amino acids at positions 5-10 of SEQ ID NO: 867 had no influence on the binding activity against IgE antibodies from allergic patients when substituted by alanine.

It was revealed for SEQ ID NO: 869 that the amino acids at positions 1, 3 and 15 are amino acids particularly important for the binding to IgE antibodies from allergic patients. The amino acids at positions 2 and 4-14 of SEQ ID NO: 869 had no influence on the binding activity against IgE antibodies from allergic patients when substituted by alanine.

It was revealed for SEQ ID NO: 871 that the amino acids at positions 8, 9 and 15 are amino acids particularly important for the binding to IgE antibodies from allergic patients. The amino acids at positions 1-7 and 10-14 of SEQ ID NO: 871 had no influence on the binding activity against IgE antibodies from allergic patients when substituted by alanine.

It was revealed for SEQ ID NO: 874 that the amino acids at positions 4-8 and 10 are amino acids particularly important for the binding to IgE antibodies from allergic patients, and the amino acids at positions 1-3 are amino acids that influence the binding to IgE antibodies from allergic patients. The amino acid at position 9 of SEQ ID NO: 874 had no influence on the binding activity against IgE antibodies from allergic patients when substituted by alanine.

It was revealed for SEQ ID NO: 877 that the amino acids at positions 1-6, 9 and 10 are amino acids particularly important for the binding to IgE antibodies from allergic patients, and the amino acids at positions 7 and 8 are amino acids that influence the binding to IgE antibodies from allergic patients. The amino acids at positions 11-15 of SEQ ID NO: 877 had no influence on the binding activity against IgE antibodies from allergic patients when substituted by alanine.

It was revealed for SEQ ID NO: 880 that the amino acids at positions 2, 4-6, 8 and 9 are amino acids particularly important for the binding to IgE antibodies from allergic patients, and the amino acids at positions 7 and 12-15 are amino acids that influence the binding to IgE antibodies from allergic patients. The amino acids at positions 1, 3, 10 and 11 of SEQ ID NO: 880 had no influence on the binding activity against IgE antibodies from allergic patients when substituted by alaninc.

It was revealed for SEQ ID NO: 882 that the amino acids at positions 1 and 5-9 are amino acids particularly important for the binding to IgE antibodies from allergic patients. The amino acids at positions 2-4 and 10 of SEQ ID NO: 882 had no influence on the binding activity against IgE antibodies from allergic patients when substituted by alanine.

It was revealed for SEQ ID NO: 884 that the amino acids at positions 5-10 are amino acids particularly important for the binding to IgE antibodies from allergic patients. The amino acids at positions 1˜4 of SEQ ID NO: 884 had no influence on the binding activity against IgE antibodies from allergic patients when substituted by alaninc.

It was revealed for SEQ ID NO: 887 that the amino acids at positions 2, 4, 5 and 14 are amino acids particularly important for the binding to IgE antibodies from allergic patients, and the amino acids at positions 6, 10, 12, 13 and 15 are amino acids that influence the binding to IgE antibodies from allergic patients. The amino acids at positions 1, 3, 7-9 and 11 of SEQ ID NO: 887 had no influence on the binding activity against IgE antibodies from allergic patients when substituted by alaninc.

It was revealed for SEQ ID NO: 889 that the amino acids at positions 3-7 are amino acids particularly important for the binding to IgE antibodies from allergic patients. The amino acids at positions 1, 2 and 8-10 of SEQ ID NO: 889 had no influence on the binding activity against IgE antibodies from allergic patients when substituted by alanine.

It was revealed for SEQ ID NO: 892 that the amino acids at positions 5-10 are amino acids particularly important for the binding to IgE antibodies from allergic patients, and the amino acids at positions 3 and 4 are amino acids that influence the binding to IgE antibodies from allergic patients. The amino acids at positions 1 and 2 of SEQ ID NO: 892 had no influence on the binding activity against IgE antibodies from allergic patients when substituted by alanine.

### (39β) Epitope of ATP Synthase Subunit O, Mitochondrial-Like (Spot 39α)

SEQ ID NO: 895 was identified as an epitope of ATP synthase subunit O, mitochondrial-like by the procedure of (B) mentioned above.

Amino acids important for binding to IgE antibodies from allergic patients were identified by the procedure of (C) mentioned above as to SEQ ID NO: 895.

It was revealed for SEQ ID NO: 895 that the amino acids at positions 2, 5, 7, and 12-14 are amino acids particularly important for the binding to IgE antibodies from allergic patients, and the amino acid at position 3 is an amino acid that influences the binding to IgE antibodies from allergic patients. The amino acids at positions 1, 4, 6, 8-11, and 15 of SEQ ID NO: 895 had no influence on the binding activity against IgE antibodies from allergic patients when substituted by alanine.

### (40β) Epitope of P24 Oleosin Isoform a (Spots 40α and 56α)

SEQ ID NOs: 898, 899, and 902 were identified as epitopes of P24 oleosin isoform A by the procedure of (B) mentioned above.

Amino acids important for binding to IgE antibodies from allergic patients were identified by the procedure of (C) mentioned above as to each of SEQ ID NOs: 898, 899 and 902.

It was revealed for SEQ ID NO: 898 that the amino acids at positions 1, 2, 4 and 5 are amino acids particularly important for the binding to IgE antibodies from allergic patients, and the amino acid at position 6 is an amino acid that influences the binding to IgE antibodies from allergic patients. The amino acid at position 3 of SEQ ID NO: 898 had no influence on the binding activity against IgE antibodies from allergic patients when substituted by alanine.

It was revealed for SEQ ID NO: 902 that the amino acids at positions 6-8 are amino acids particularly important for the binding to IgE antibodies from allergic patients, and the amino acid at position 1 is an amino acid that influences the binding to IgE antibodies from allergic patients. The amino acids at positions 2-5 and 9 of SEQ ID NO: 902 had no influence on the binding activity against IgE antibodies from allergic patients when substituted by alanine.

For SEQ ID NO: 899, all the amino acids were amino acids particularly important for the binding to IgE antibodies from allergic patients.

### (41β) Epitope of Uncharacterized Protein At3g49720-Like Isoform X2 (Spot 41α)

SEQ ID NO: 903 was identified as an epitope of uncharacterized protein At3g49720-like isoform X2 by the procedure of (B) mentioned above.

Amino acids important for binding to IgE antibodies from allergic patients were identified by the procedure of (C) mentioned above as to SEQ ID NO: 903.

For SEQ ID NO: 903, all the amino acids were amino acids particularly important for the binding to IgE antibodies from allergic patients.

### (42) Epitope of Superoxide Dismutase [Mn], Mitochondrial (Spots 42α and 43α)

SEQ ID NO: 906 was identified as an epitope of superoxide dismutase [Mn], mitochondrial by the procedure of (B) mentioned above.

Amino acids important for binding to IgE antibodies from allergic patients were identified by the procedure of (C) mentioned above as to SEQ ID NO: 906.

It was revealed for SEQ ID NO: 906 that the amino acids at positions 3 and 11-13 are amino acids particularly important for the binding to IgE antibodies from allergic patients, and the amino acids at positions 8 and 14 are amino acids that influence the binding to IgE antibodies from allergic patients. The amino acids at positions 1, 2, 4-7, 9, 10, and 15 of SEQ ID NO: 906 had no influence on the binding activity against IgE antibodies from allergic patients when substituted by alanine.

### (44β) Epitope of Ferritin-2 (Spot 44α)

SEQ ID NO: 909 was identified as an epitope of Ferritin-2 by the procedure of (B) mentioned above.

Amino acids important for binding to IgE antibodies from allergic patients were identified by the procedure of (C) mentioned above as to SEQ ID NO: 909.

It was revealed for SEQ ID NO: 909 that the amino acids at positions 5-11 and 13-15 are amino acids particularly important for the binding to IgE antibodies from allergic patients. The amino acids at positions 1˜4 and 12 of SEQ ID NO: 909 had no influence on the binding activity against IgE antibodies from allergic patients when substituted by alanine.

### (45β) Epitope of Uncharacterized Protein LOC100499771 (Spot 45α)

SEQ ID NOs: 910-912 were identified as epitopes of uncharacterized protein LOC100499771 by the procedure of (B) mentioned above.

Amino acids important for binding to IgE antibodies from allergic patients were identified by the procedure of (C) mentioned above as to each of SEQ ID NOs: 910-912.

For SEQ ID NOs: 910-912, all the amino acids were amino acids particularly important for the binding to IgE antibodies from allergic patients.

### (46β) Epitope of Seed Maturation Protein PM22 (Spot 46α)

SEQ ID NO: 915 was identified as an epitope of seed maturation protein PM22 by the procedure of (B) mentioned above.

Amino acids important for binding to IgE antibodies from allergic patients were identified by the procedure of (C) mentioned above as to SEQ ID NO: 915.

It was revealed for SEQ ID NO: 915 that the amino acids at positions 2, 4, and 8 are amino acids particularly important for the binding to IgE antibodies from allergic patients, and the amino acid at position 3 is an amino acid that influences the binding to IgE antibodies from allergic patients. The amino acids at positions 1, 5-7, and 9 of SEQ ID NO: 915 had no influence on the binding activity against IgE antibodies from allergic patients when substituted by alanine.

### (47β) Epitope of Eukaryotic Translation Initiation Factor 5A-2 (Spot 47α)

SEQ ID NOs: 916-918 were identified as epitopes of eukaryotic translation initiation factor 5A-2 by the procedure of (B) mentioned above.

Amino acids important for binding to IgE antibodies from allergic patients were identified by the procedure of (C) mentioned above as to each of SEQ ID NOs: 916-918.

For SEQ ID NOs: 916-918, all the amino acids were amino acids particularly important for the binding to IgE antibodies from allergic patients.

### (48β) Epitope of Uncharacterized Protein LOC100306570 (Spot 48α)

SEQ ID NOs: 921 and 922 were identified as epitopes of uncharacterized protein LOC100306570 by the procedure of (B) mentioned above.

Amino acids important for binding to IgE antibodies from allergic patients were identified by the procedure of (C) mentioned above as to each of SEQ ID NOs: 921 and 922.

It was revealed for SEQ ID NO: 921 that the amino acids at positions 7-9 are amino acids particularly important for the binding to IgE antibodies from allergic patients, and the amino acid at position 5 is an amino acid that influences the binding to IgE antibodies from allergic patients. The amino acids at positions 1˜4 and 6 of SEQ ID NO: 921 had no influence on the binding activity against IgE antibodies from allergic patients when substituted by alanine.

For SEQ ID NO: 922, all the amino acids were amino acids particularly important for the binding to IgE antibodies from allergic patients.

### (49β) Epitope of Bowman-Birk Type Proteinase Inhibitor D-II (Spot 49α)

SEQ ID NO: 923 was identified as an epitope of Bowman-Birk type proteinase inhibitor D-II by the procedure of (B) mentioned above.

Amino acids important for binding to IgE antibodies from allergic patients were identified by the procedure of (C) mentioned above as to SEQ ID NO: 923.

For SEQ ID NO: 923, all the amino acids were amino acids particularly important for the binding to IgE antibodies from allergic patients.

### (50β) Epitope of Uncharacterized Protein LOC100813859 (Spot 50α)

SEQ ID NOs: 924 and 927 were identified as epitopes of uncharacterized protein LOC100813859 by the procedure of (B) mentioned above.

Amino acids important for binding to IgE antibodies from allergic patients were identified by the procedure of (C) mentioned above as to each of SEQ ID NOs: 924 and 927.

It was revealed for SEQ ID NO: 927 that the amino acids at positions 5 and 6 are amino acids particularly important for the binding to IgE antibodies from allergic patients, and the amino acid at position 3 is an amino acid that influences the binding to IgE antibodies from allergic patients. The amino acids at positions 1, 2, 4, and 7-9 of SEQ ID NO: 927 had no influence on the binding activity against IgE antibodies from allergic patients when substituted by alanine.

For SEQ ID NO: 924, all the amino acids were amino acids particularly important for the binding to IgE antibodies from allergic patients.

### (51) Epitope of 14-3-3-Like Protein (Spot 51α)

SEQ ID NOs: 928-930 were identified as epitopes of 14-3-3-like protein by the procedure of (B) mentioned above.

Amino acids important for binding to IgE antibodies from allergic patients were identified by the procedure of (C) mentioned above as to each of SEQ ID NOs: 928-930.

For SEQ ID NOs: 928-930, all the amino acids were amino acids particularly important for the binding to IgE antibodies from allergic patients.

### (52β) Epitope of Probable Fructokinase-4 (Spot 52α)

SEQ ID NOs: 933 and 934-938 were identified as epitopes of probable fructokinase-4 by the procedure of (B) mentioned above.

Amino acids important for binding to IgE antibodies from allergic patients were identified by the procedure of (C) mentioned above as to each of SEQ ID NOs: 933 and 934-938.

It was revealed for SEQ ID NO: 933 that the amino acids at positions 7, 10, and 12 are amino acids particularly important for the binding to IgE antibodies from allergic patients, and the amino acid at position 3 is an amino acid that influences the binding to IgE antibodies from allergic patients. The amino acids at positions 1, 2, 4-6, 8, 9, 11, and 13-15 of SEQ ID NO: 933 had no influence on the binding activity against IgE antibodies from allergic patients when substituted by alanine.

For SEQ ID NOs: 934-938, all the amino acids were amino acids particularly important for the binding to IgE antibodies from allergic patients.

### (53β) Epitope of Triosephosphate Isomerase Isoform X1 (Spot 53α)

SEQ ID NO: 939 was identified as an epitope of triosephosphate isomerase isoform X1 by the procedure of (B) mentioned above.

Amino acids important for binding to IgE antibodies from allergic patients were identified by the procedure of (C) mentioned above as to SEQ ID NO: 939.

For SEQ ID NO: 939, all the amino acids were amino acids particularly important for the binding to IgE antibodies from allergic patients.

### (54β) Epitope of Superoxide Dismutase [Fe], Chloroplastic Isoform X1 (Spot 54α)

SEQ ID NOs: 940 and 943 were identified as epitopes of superoxide dismutase [Fe], chloroplastic isoform X1 by the procedure of (B) mentioned above.

Amino acids important for binding to IgE antibodies from allergic patients were identified by the procedure of (C) mentioned above as to each of SEQ ID NOs: 940 and 943.

It was revealed for SEQ ID NO: 943 that the amino acids at positions 1 and 6 are amino acids particularly important for the binding to IgE antibodies from allergic patients, and the amino acid at position 7 is an amino acid that influences the binding to IgE antibodies from allergic patients. The amino acids at positions 2-5 of SEQ ID NO: 943 had no influence on the binding activity against IgE antibodies from allergic patients when substituted by alanine.

For SEQ ID NO: 940, all the amino acids were amino acids particularly important for the binding to IgE antibodies from allergic patients.

### (55β) Epitope of 51 kDa Seed Maturation Protein Precursor (Spot 55α)

SEQ ID NOs: 946, 949, and 952 were identified as epitopes of 51 kDa seed maturation protein precursor by the procedure of (B) mentioned above.

Amino acids important for binding to IgE antibodies from allergic patients were identified by the procedure of (C) mentioned above as to each of SEQ ID NOs: 946, 949, and 952.

It was revealed for SEQ ID NO: 946 that the amino acids at positions 1, 2, 6, and 7 are amino acids particularly important for the binding to IgE antibodies from allergic patients, and the amino acid at position 3 is an amino acid that influences the binding to IgE antibodies from allergic patients. The amino acids at positions 4, 5, 8, and 9 of SEQ ID NO: 946 had no influence on the binding activity against IgE antibodies from allergic patients when substituted by alanine.

It was revealed for SEQ ID NO: 949 that the amino acids at positions 1-3, 7, and 8 are amino acids particularly important for the binding to IgE antibodies from allergic patients, and the amino acid at position 4 is an amino acid that influences the binding to IgE antibodies from allergic patients. The amino acids at positions 5 and 6 of SEQ ID NO: 949 had no influence on the binding activity against IgE antibodies from allergic patients when substituted by alanine.

It was revealed for SEQ ID NO: 952 that the amino acids at positions 1, 2, and 9 are amino acids particularly important for the binding to IgE antibodies from allergic patients, and the amino acid at position 8 is an amino acid that influences the binding to IgE antibodies from allergic patients. The amino acids at positions 3-7 of SEQ ID NO: 952 had no influence on the binding activity against IgE antibodies from allergic patients when substituted by alanine.

### Example 5: Identification of Epitopes (2)

Identification of epitopes was carried out by the same procedure as in Example 4 as to each of the following antigen proteins.

### (57) Epitope of Gly m 4 (UniProt Accession Number: P26987.1)

SEQ ID NO: 955 was identified as an epitope of Gly m 4 by the procedure of (B) of Example 4.

Amino acids important for binding to IgE antibodies from allergic patients were identified by the procedure of (C) of Example 4 as to SEQ ID NO: 955.

It was revealed for SEQ ID NO: 955 that the amino acids at positions 8, 10, 12, 14, and 15 are amino acids particularly important for the binding to IgE antibodies from allergic patients, and the amino acids at positions 7, 9, 11, and 13 are amino acids that influence the binding to IgE antibodies from allergic patients. The amino acids at positions 1-6 of SEQ ID NO: 955 had no influence on the binding activity against IgE antibodies from allergic patients when substituted by alanine.

### (58β) Epitopes of Gly m 5a (NCBI Accession Number: NP_001236856.1) and Gly m 5α′ (NCBI Accession Number: NP 001237316.1)

SEQ ID NOs: 956-960 were identified as epitopes of Gly m 5α or Gly m 5α′ by the procedure of (B) of Example 4.

Amino acids important for binding to IgE antibodies from allergic patients were identified by the procedure of (C) of Example 4 as to each of SEQ ID NOs: 956-960.

For SEQ ID NOs: 956-960, all the amino acids were amino acids particularly important for the binding to IgE antibodies from allergic patients.

### (59β) Epitopes of Gly m 6.01 (UniProt Accession Number: CAA33215.1) and Gly m 6.02 (UniProt Accession Number: BAA00154.1)

SEQ ID NOs: 961 and 962 were identified as epitopes of Gly m 6.01 or Gly m 6.02 by the procedure of (B) of Example 4.

Amino acids important for binding to IgE antibodies from allergic patients were identified by the procedure of (C) of Example 4 as to each of SEQ ID NOs: 961 and 962.

For SEQ ID NOs: 961 and 962, all the amino acids were amino acids particularly important for the binding to IgE antibodies from allergic patients.

### (60β) Epitope of Gly m 8 (UniProt Accession Number: AAB71140.1)

SEQ ID NO: 963 was identified as an epitope of Gly m 8 by the procedure of (B) of Example 4.

Amino acids important for binding to IgE antibodies from allergic patients were identified by the procedure of (C) of Example 4 as to SEQ ID NO: 963.

For SEQ ID NO: 963, all the amino acids were amino acids particularly important for the binding to IgE antibodies from allergic patients.

### Example 6: Study on Cross-Reactivity

Amino acids important for binding activity against IgE antibodies from allergic patients were identified as to SEQ ID NO: 871, one of the polypeptides in spot 38β, to obtain XXXXXXXRRXXXXXR (hereinafter also referred to as a key sequence). A protein having this key sequence was compared against NCBI protein data and analyzed by PHI-BLAST. As a result, peach-derived kinesin-like protein KIN-7C comprising SEQ ID NO: 984 was identified as an amino acid sequence corresponding to the key sequence, and apple-derived uncharacterized protein LOC103433727 isoform X2 comprising SEQ ID NO: 985 was identified as an amino acid sequence corresponding to the key sequence.

The binding activity of peptides comprising the amino acid sequences of SEQ ID NOS: 871, 984 and 985 against an IgE antibody from one patient having allergies to a soybean, a peach and an apple was confirmed by ELISA. The peptides were synthesized such that the peptides were N-terminally biotinylated by the Fmoc method.

To be specific, ELISA was carried out according to the following procedure.
(1) The concentrations of the biotinylated peptides were adjusted to 1 μg/mL with PBST.
(2) Each peptide solution was added at 40 μL to each well of a 384-well plate coated with streptavidin, and shaken at room temperature for one hour. After collection of the solution, the wells were washed with PBS five times.
(3) A 1:5 dilution of Blocking one (diluted with MQ) was added at 40 μL thereto and shaken at room temperature for 15 minutes. After removal of the solution, the wells were washed with PBS five times.
(4) A diluted serum solution (1:10, diluting solution: Blocking one produced by Nacalai Tesque, Inc.) was added at 40 μL thereto and shaken at room temperature for one hour. After removal of the solution, the wells were washed with PBS five times.
(5) A diluted secondary antibody solution (1:5000, diluting solution: Blocking one produced by Nacalai Tesque, Inc.) was added at 40 μL thereto and shaken at room temperature for one hour. After removal of the solution, the wells were washed with PBS five times.
(6) 1-Step Ultra TMB-ELISA (Thermo Fisher Scientific) was added at 40 μL thereto and shaken at room temperature for 15 minutes.
(7) 2 M H₂SO₄ was added at 40 μL thereto. Absorbance at 450 nm was measured.

The results are shown in FIG. 6. As shown in FIG. 6, the peptides comprising the amino acid sequences of SEQ ID NOs: 871, 984 and 985 exhibited binding activity against the IgE antibody from the patient having allergies to a soybean, a peach and an apple. Also, these peptides bound more strongly to the IgE antibody from the allergic patient than to an IgE antibody from a healthy subject who was not an allergic patient. This indicates that this key sequence can be used for detecting the cross-reactivity of an allergen.

This result also indicates that the polypeptides (1β) to (60β) of the present invention can also be used for the detection of the cross-reactivity of an allergen.

1-11. (canceled)

12. A method for detecting binding between an antigen and an antibody from a sample obtained from a subject,
wherein the antigen is a polypeptide selected from one of the following:
(1β) a polypeptide comprising at least one amino acid sequence selected from the group consisting of SEQ ID NO: 660;
(2β) a polypeptide comprising at least one amino acid sequence selected from the group consisting of SEQ ID NOs: 663, 666, 669-671, 675-678 and 681, 684, 689-691;
(3β) a polypeptide comprising at least one amino acid sequence selected from the group consisting of SEQ ID NOs: 695, 697 and 699-716;
(9β) a polypeptide comprising at least one amino acid sequence selected from the group consisting of SEQ ID NOs: 747-749;
(14β) a polypeptide comprising at least one amino acid sequence selected from the group consisting of SEQ ID NOs: 761, 764;
(25β) a polypeptide comprising at least one amino acid sequence selected from the group consisting of SEQ ID NOs: 814 and 815; and
(26β) a polypeptide comprising at least one amino acid sequence selected from the group consisting of SEQ ID NOs: 816, 819;
wherein the polypeptide as defined in (1β), (2β), (3β), (9β), (14β), (25β), and (26β) specifically binds to an IgE antibody from a patient allergic to soybean, and
wherein the method comprises contacting the antibody in a sample obtained from the subject that binds to the antigen with the antigen from a kit.

13. The method according to claim 12, wherein the antigen from the kit is immobilized to a carrier or a surface usable for detection of binding between the antibody and the polypeptide.

14. The method according to claim 12, wherein the kit further comprises a reagent that detects binding between said antigen and said antibody in the sample obtained from the subject.

15. The method according to claim 12, wherein the polypeptide being any one of the following:
(2β-A) a polypeptide comprising at least one amino acid sequence selected from the group consisting of SEQ ID NOs: 666, 670-671, 675-678, 681, 684 and 689;
(3-A) a polypeptide comprising at least one amino acid sequence selected from the group consisting of SEQ ID NOs: 695, 697, 703, 704, 707, 709-711, 713, 715 and 716;
(14β-A) a polypeptide comprising at least one amino acid sequence selected from the group consisting of SEQ ID NOs: 761, 764;
(25β-A) a polypeptide comprising at least one amino acid sequence selected from the group consisting of SEQ ID NOs: 814 and 815; and
(26β-A) a polypeptide comprising at least one amino acid sequence selected from the group consisting of SEQ ID NO: 816.

16. The method according to claim 12, wherein the polypeptide being any one of the following:
(1β-B1) a polypeptide consisting of an amino acid sequence having at least 90% identity to the amino acid sequence of SEQ ID NO: 70 or 89 and further comprising at least one amino acid sequence selected from the group consisting of SEQ ID NO: 660;
(2β-B1) a polypeptide consisting of an amino acid sequence having at least 90% identity to the amino acid sequence of SEQ ID NO: 106 or 139 or consisting of an amino acid sequence having at least 75% identity to the amino acid sequence of SEQ ID NO: 122, and further comprising at least one amino acid sequence selected from the group consisting of SEQ ID NOs: 663, 666, 669-671, 675-678 and 681, 684, 689-691;
(3β-B1) a polypeptide consisting of an amino acid sequence having at least 90% identity to the amino acid sequence of SEQ ID NO: 16 and further comprising at least one amino acid sequence selected from the group consisting of SEQ ID NOs: 695, 697 and 699-716;
(9β-B1) a polypeptide consisting of an amino acid sequence having at least 90% identity to the amino acid sequence of SEQ ID NO: 201 and further comprising at least one amino acid sequence selected from the group consisting of SEQ ID NOs: 747-749;
(14β-B1) a polypeptide consisting of an amino acid sequence having at least 90% identity to the amino acid sequence of SEQ ID NO: 280 or 286 and further comprising at least one amino acid sequence selected from the group consisting of SEQ ID NOs: 761, 764;
(25β-B1) a polypeptide consisting of an amino acid sequence having at least 90% identity to the amino acid sequence of SEQ ID NO: 348 or 358 and further comprising at least one amino acid sequence selected from the group consisting of SEQ ID NOs: 814 and 815; and
(26β-B1) a polypeptide consisting of an amino acid sequence having at least 90% identity to the amino acid sequence of SEQ ID NO: 367, 384 or 394 and further comprising at least one amino acid sequence selected from the group consisting of SEQ ID Nos 816, 819.

17. The method according to claim 12, wherein the sample is obtained from the subject, wherein the method further comprises detecting binding between an IgE antibody present in the sample and the antigen from the kit, and wherein the antigen from the kit is immobilized to a carrier or a surface.

18. The method according to claim 12, wherein the sample is obtained from the subject, wherein the method further comprises detecting binding between an IgE antibody present in the sample and the antigen from the kit, and wherein the kit further comprises a reagent that detects binding between said antigen and said antibody.

19. A method for diagnosing and treating an allergy to soybean in a subject, wherein the method comprises:
(i) detecting binding between an antibody in a sample obtained from the subject with and an antigen from a kit,
wherein the antigen is a polypeptide selected from one of the following:
(1β) a polypeptide comprising at least one amino acid sequence selected from the group consisting of SEQ ID NO: 660;
(2β) a polypeptide comprising at least one amino acid sequence selected from the group consisting of SEQ ID NOs: 663, 666, 669-671, 675-678 and 681, 684, 689-691;
(3β) a polypeptide comprising at least one amino acid sequence selected from the group consisting of SEQ ID NOs: 695, 697 and 699-716;
(9β) a polypeptide comprising at least one amino acid sequence selected from the group consisting of SEQ ID NOs: 747-749;
(14β) a polypeptide comprising at least one amino acid sequence selected from the group consisting of SEQ ID NOs: 761, 764;
(25β) a polypeptide comprising at least one amino acid sequence selected from the group consisting of SEQ ID NOs: 814 and 815; and
(26β) a polypeptide comprising at least one amino acid sequence selected from the group consisting of SEQ ID NOs: 816, 819;
wherein the polypeptide as defined in (1β), (2β), (3β), (9β), (14), (25β), and (26β) specifically binds to an IgE antibody from a patient allergic to soybean, and
(ii) administering hyposensitization therapy to the subject when binding between said antibody and the antigen is detected.

20. The method according to claim 19, wherein the antigen from the kit is immobilized to a carrier or a surface usable for detection of binding between the antibody and the polypeptide.

21. The method according to claim 19, wherein the kit further comprises a reagent that detects binding between said antigen and said antibody in the sample obtained from the subject.

22. The method according to claim 19, wherein the polypeptide being any one of the following:
(2β-A) a polypeptide comprising at least one amino acid sequence selected from the group consisting of SEQ ID NOs: 666, 670-671, 675-678, 681, 684 and 689;
(3-A) a polypeptide comprising at least one amino acid sequence selected from the group consisting of SEQ ID NOs: 695, 697, 703, 704, 707, 709-711, 713, 715 and 716;
(14β-A) a polypeptide comprising at least one amino acid sequence selected from the group consisting of SEQ ID NOs: 761, 764;
(25β-A) a polypeptide comprising at least one amino acid sequence selected from the group consisting of SEQ ID NOs: 814 and 815; and
(26β-A) a polypeptide comprising at least one amino acid sequence selected from the group consisting of SEQ ID NO: 816.

23. The method according to claim 19, wherein the polypeptide being any one of the following:
(1β-B1) a polypeptide consisting of an amino acid sequence having at least 90% identity to the amino acid sequence of SEQ ID NO: 70 or 89 and further comprising at least one amino acid sequence selected from the group consisting of SEQ ID NO: 660;
(2β-B1) a polypeptide consisting of an amino acid sequence having at least 90% identity to the amino acid sequence of SEQ ID NO: 106 or 139 or consisting of an amino acid sequence having at least 75% identity to the amino acid sequence of SEQ ID NO: 122, and further comprising at least one amino acid sequence selected from the group consisting of SEQ ID NOs: 663, 666, 669-671, 675-678 and 681, 684, 689-691;
(3β-B1) a polypeptide consisting of an amino acid sequence having at least 90% identity to the amino acid sequence of SEQ ID NO: 16 and further comprising at least one amino acid sequence selected from the group consisting of SEQ ID NOs: 695, 697 and 699-716;
(9β-B1) a polypeptide consisting of an amino acid sequence having at least 90% identity to the amino acid sequence of SEQ ID NO: 201 and further comprising at least one amino acid sequence selected from the group consisting of SEQ ID NOs: 747-749;
(14β-B1) a polypeptide consisting of an amino acid sequence having at least 90% identity to the amino acid sequence of SEQ ID NO: 280 or 286 and further comprising at least one amino acid sequence selected from the group consisting of SEQ ID NOs: 761, 764;
(25β-B1) a polypeptide consisting of an amino acid sequence having at least 90% identity to the amino acid sequence of SEQ ID NO: 348 or 358 and further comprising at least one amino acid sequence selected from the group consisting of SEQ ID NOs: 814 and 815; and
(26β-B1) a polypeptide consisting of an amino acid sequence having at least 90% identity to the amino acid sequence of SEQ ID NO: 367, 384 or 394 and further comprising at least one amino acid sequence selected from the group consisting of SEQ ID Nos 816, 819.


SEQUENCE LISTING (985 sequences)

SEQ ID NO: 1 - DNA, 249 bp, Glycine max
  source 1..249: mol_type = other DNA
atgtgtattctgagcttcctcaaaagtgatcagtcatcaagttatgatgatgatgagtattcaaaaccatgctgtgatctctgcatgtgcacacgctcaatgcctcctcaatgcagctgtgaagatattaggctgaattcatgccactcagattgtaagagctgtatgtgcacacgctcacagccaggacagtgtcgttgtcttgacaccaacgacttctgctacaaaccttgcaagtccagagatgac

SEQ ID NO: 2 - protein, 83 aa, Glycine max
MCILSFLKSDQSSSYDDDEYSKPCCDLCMCTRSMPPQCSCEDIRLNSCHSDCKSCMCTRSQPGQCRCLDTNDFCYKPCKSRDD

SEQ ID NO: 3 - DNA, 1425 bp, Glycine max
  source 1..1425: mol_type = other DNA
atgtcaccacaaacagagactaaagcaagtgttgggttcaaagctggtgttaaagattataaattgacttattatactcctgactatgaaaccaaagatactgatatcttggcagcattccgagtaactcctcaaccaggagttccgcctgaagaagcaggtgccgcggtagccgccgaatcttctactggtacatggacaactgtgtggaccgatgggcttaccagtcttgatcgttacaaagggcgatgctacggccttgaacctgttgctggggaagaaaatcaatatattgcttatgtagcttatcccttagacctttttgaagaaggttctgttactaacatgtttacttccattgtcggtaatgtatttgggttcaaggccctgcgtgctctacgtctggaggatttgcgaatccctactgcttatattaaaactttccaaggtccgcctcatggcatccaagttgagagagataaattgaacaagtatggtcgtcccctattaggatgtactattaaacctaaattggggttatccgctaagaattatggtagagctgtttatgaatgtcttcgtgggggacttgattttaccaaagatgatgaaaatgtgaattcccaaccatttatgcgttggagagaccgtttcttattttgtgccgaagccatttttaaatcacaggctgaaacaggtgaaatcaaagggcattacttgaatgcaactgcgggtacatgcgaagaaatgatgaaaagagctgtatttgccagagaattaggcgttcctatcgtaatgcatgattatttaacggggggattcactgcaaatactagcttggctcattattgccgagataatggtctacttcttcatatacaccgtgcaatgcatgcagttatcgacagacaaaagaatcatggtatgcactttcgtgtactagctaaagcattacgtttgtctggtggagatcatgttcacgccggtaccgtagtaggtaaacttgaaggggaaagagaaatcactttaggttttgttgatttactacgtgatgattttgttgaaaaagatcgaagtcgcggtatttatttcactcaggattgggtttctctaccaggtgttttgcctgtagcttcgggaggtattcacgtttggcatatgcctgctctgaccgagatctttggggatgattctgtactccaatttggcggaggaactttaggacacccttggggaaatgcaccaggtgctgtagctaatcgagtagctcttgaagcatgtgtgcaggctcgaaatgaaggacgcgatcttgctcgtgaaggtaatgaaattatccgtgaggctagcaaatggagtcctgaattagctgctgcttgtgaagtatggaaggagatcaaatttgaattcgaagcaatggatactttg

SEQ ID NO: 4 - protein, 475 aa, Glycine max
MSPQTETKASVGFKAGVKDYKLTYYTPDYETKDTDILAAFRVTPQPGVPPEEAGAAVAAESSTGTWTTVWTDGLTSLDRYKGRCYGLEPVAGEENQYIAYVAYPLDLFEEGSVTNMFTSIVGNVFGFKALRALRLEDLRIPTAYIKTFQGPPHGIQVERDKLNKYGRPLLGCTIKPKLGLSAKNYGRAVYECLRGGLDFTKDDENVNSQPFMRWRDRFLFCAEAIFKSQAETGEIKGHYLNATAGTCEEMMKRAVFARELGVPIVMHDYLTGGFTANTSLAHYCRDNGLLLHIHRAMHAVIDRQKNHGMHFRVLAKALRLSGGDHVHAGTVVGKLEGEREITLGFVDLLRDDFVEKDRSRGIYFTQDWVSLPGVLPVASGGIHVWHMPALTEIFGDDSVLQFGGGTLGHPWGNAPGAVANRVALEACVQARNEGRDLAREGNEIIREASKWSPELAAACEVWKEIKFEFEAMDTL

SEQ ID NO: 5 - DNA, 1026 bp, Glycine max
  source 1..1026: mol_type = other DNA
atgccaaacgacagcgttctgagtcttttcttcttcgttaccctcttcacatgccttctctctgctacttcccacgatgaccatattttcctaccgagtcaactccatgacgatgactcagtgagttgcactgcaaccgacccaagccttaactacaagccggtgatcggaatcctgacccaccccggcgacggtgcctctggccggctcagcaacgccaccggggtttcttacatcgccgcctcctacgttaaattcgtggagtccggtggtgctagagttattccccttatctacaatgagtcacctgagaaccttaacaagaagctcgatttggtcaatggagtgctcttcactggtgggtgggctgtttctggtccatatttggatactcttggaaacatttttaagaaagctttagagagaaatgatgctggagaccatttcccagtaattgccttcaacttaggtggaaacctcgtaataaggatcgtaagcgagcaaactgatattcttgaaccatttactgcgtcgagtttgccaagttctcttgtattgtggaacgaagcaaatgctaaaggaagtctatttcaaagatttccttcagatttgttgacacagctgaagacagattgtctcgtcctgcacaatcatcgttatgccatatccccacgcaagcttcaatataataccaagttatctgacttttttgagattctggcaacatctggggatagagatggaaagactttcgtttccacagcacgtggtaggaaatatcccgtgactgtcaacctatggcaaccagagaaaaatgcgtttgagtgggctacatctttgaaagcccctcacacagaggatgcaattcgggttactcagtccacagcaaactttttcataagtgaagctaggaaatccacaaacacacctgatgctcaaaaagtgcgagatagtctcatctacaattacaaacctacttttggtggaacggctgggaaaggatatgatcaagtgtatctctttgaa

SEQ ID NO: 6 - protein, 342 aa, Glycine max
MPNDSVLSLFFFVTLFTCLLSATSHDDHIFLPSQLHDDDSVSCTATDPSLNYKPVIGILTHPGDGASGRLSNATGVSYIAASYVKFVESGGARVIPLIYNESPENLNKKLDLVNGVLFTGGWAVSGPYLDTLGNIFKKALERNDAGDHFPVIAFNLGGNLVIRIVSEQTDILEPFTASSLPSSLVLWNEANAKGSLFQRFPSDLLTQLKTDCLVLHNHRYAISPRKLQYNTKLSDFFEILATSGDRDGKTFVSTARGRKYPVTVNLWQPEKNAFEWATSLKAPHTEDAIRVTQSTANFFISEARKSTNTPDAQKVRDSLIYNYKPTFGGTAGKGYDQVYLFE

SEQ ID NO: 7 - DNA, 975 bp, Glycine max
  source 1..975: mol_type = other DNA
atggcggaaggactcgtcctgaagggaaccatgcgcgcccacaccgacgtcgtgacggcgattgccactcccatcgacaactccgacatgatcgtgacggcgtcgcgcgacagatccatcattctctggcacctcaccaaggaggacaagacctacggtgtcccccgccgccgcctcaccggacattctcacttcgtccaggacgtcgtcctctcatccgacggtcagttcgccctctccggctcctgggacggcgagctccgcctctgggacctcgcggctggcacctctgcccgccgcttcgttggccacaccaaggacgtgctctccgtggcgttctccatcgacaaccgtcagatcgtgtcggcctctcgtgaccgcacgatcaagctgtggaacaccctgggtgagtgcaagtacacaatccaagatggcgatgcgcattcggattgggtaagttgcgtccgtttcagccctagcactcttcagccaaccattgtttctgcttcatgggacaggaccgttaaggtttggaacctgaccaactgcaagctgaggaacacccttgctggacacaatgggtatgtgaatactgttgctgtttcccctgatggctctctctgtgccagtggtggcaaagatggggttattcttctgtgggatttggctgagggtaagcgtctttactctctcgatgctggctcaatcatccatgcactctgcttcagccccagcaggtactggctctgcgccgccaccgagcagagcatcaagatctgggatttggagagcaagagtatcgttgaggatttgaaggttgacctcaagactgaggctgatgccacctccggtggtggtaacgccaacaagaagaaggttatttattgcacaagtttgaactggagtgcggatggaagcactttgtttagtggctataccgatggtgtggccagagtttgggctattggacgttat

SEQ ID NO: 8 - protein, 325 aa, Glycine max
MAEGLVLKGTMRAHTDVVTAIATPIDNSDMIVTASRDRSIILWHLTKEDKTYGVPRRRLTGHSHFVQDVVLSSDGQFALSGSWDGELRLWDLAAGTSARRFVGHTKDVLSVAFSIDNRQIVSASRDRTIKLWNTLGECKYTIQDGDAHSDWVSCVRFSPSTLQPTIVSASWDRTVKVWNLTNCKLRNTLAGHNGYVNTVAVSPDGSLCASGGKDGVILLWDLAEGKRLYSLDAGSIIHALCFSPSRYWLCAATEQSIKIWDLESKSIVEDLKVDLKTEADATSGGGNANKKKVIYCTSLNWSADGSTLFSGYTDGVARVWAIGRY

SEQ ID NO: 9 - DNA, 303 bp, Glycine max
  source 1..303: mol_type = other DNA
atggcatctcatcaacaaaacaagcaagagcttgatgaaagggcaaggcaaggagagactgttgttccgggtggcactggtggcaagagccttgaggctcagcaacatcttgctgaaggaaggagcaggggagggaaaacaaggaaggagcagctggggacagaagggtaccatgaaatgggacgcaaaggtgggttgagcactatggacaagtcaggagaagaacgtgctcaagaggaagccattgacatcgatgagtccaagttcaggactggtaataaccaagacaagaaccagaacaag

SEQ ID NO: 10 - protein, 101 aa, Glycine max
MASHQQNKQELDERARQGETVVPGGTGGKSLEAQQHLAEGRSRGGKTRKEQLGTEGYHEMGRKGGLSTMDKSGEERAQEEAIDIDESKFRTGNNQDKNQNK

SEQ ID NO: 11 - DNA, 1929 bp, Glycine max
  source 1..1929: mol_type = other DNA
atggcgtctgaacaattggctcgcagagagaacaccaccaccgagaaggagattcacgttgagaaacacagggttcccaagatggccacccacttcgagcaccttgcggagcaagctaaagaatctgacatcaccgctgggaaggatactccgcagggtagcatagaagccttgcaagctggagagagggtcaaagatcatgcaggaaaagccatgggagatattggtggacgtgggaaagccagagagactcacgagctgggtgcacactttgaatccctggctgacaaagtaactgaccatgcagcagccaatgtagttggaaacaaagagagtcaaagagaagctcgtggtggtgttcgtgatgttgggaagttcgaaatgagaactgaagggggagaaaaaggaaacaaggatagaccagaactgaaaacacgaaccagagaggtaataggaagaacggagaaagaaagaggaagagaaagtggaggacaagtagtggcagagaaaggaagagaaacagaaactgcaaggggaagggttggtgctgaaaatgaaggtgcaagaacaaccgctgttataacttgcacgttggaaaaaggtggtggtactcagaaaccaataagagaagaagagagagagagtgaaagtgaaagatcagcgtgggaacagatttccaactacagtgatcaagccacacaaggtgtgaaggaaaagtacgagagagcaaagcaagcagcaagcgagacactgaacaccacaacacaaaccgcacaagagaaaagcgcacaggcaaagaatcttgcagcacaagcaaaggacgcaacgttagagaagggtcagcaaggctatgctgtgacaaaggacaccatttcaagtgcagcgaaaaccgcttcggagaaaactgcgccagtggcagagaaggccaaagactacactcttcaggctgcggagaaggccaagagtgcgggtggcacaacggcgagttacgtcggagagaaagcggtgcaggccaaggatgtcgccgtggagagtgggaagagtgcggctgggtatgctgccaaggtggctgctgatttgagggacaaggccaccgcggtggggtgggccgcggcccatttttcggcggaaaagacggtggaagggaccaaggccgccgcccatgttgtcgagggagcggccgggtatgccggacataaggccgcggagcttgcttcgatgtcggccggtgctgttaaagggctggctgcttctgctggtgagactgctaaggaatacaccgcaaagaagaaagaggaggcacagcgggaattggaggccaaaaaaccatctcaacctcaggaagctgaagagaggccatcagaaggaattggcgaaactgtgaggcagtatgcacaaaaaccaaagccaagtgaaggaaaccctcagaaggagggaaccggaagcatcgtgtttacagcaataggcgaaacagtaagcagtgccggtgagaaggtaaagaaaccgtttaaaaacactatgggaggagaaagtgaaggaggaggagggaaagaagagggaaagagtgtcattggtaaaagtttgaccagcattggtgagaagctgggtgatgcaaaacagagagaggagctgttagacaacgtcactggcaacatcaccgagggaggaggcgaggtgttgggagcagtaggtgaaaccgttgctgagattggacagaacatgatgaagccagcggagatagttcaggaacgcgctcacgttcgacaagcgggtggggtcttggatgccattggtgaaaccattgctgagattgctgaaacgactcgagtcatggtttctggagaggacgagagagtgctgaggcagagcgtggtgttggagactcgcgtaactggtcgtgccaagcatgaagaagggtcgcacggtgct

SEQ ID NO: 12 - protein, 643 aa, Glycine max
MASEQLARRENTTTEKEIHVEKHRVPKMATHFEHLAEQAKESDITAGKDTPQGSIEALQAGERVKDHAGKAMGDIGGRGKARETHELGAHFESLADKVTDHAAANVVGNKESQREARGGVRDVGKFEMRTEGGEKGNKDRPELKTRTREVIGRTEKERGRESGGQVVAEKGRETETARGRVGAENEGARTTAVITCTLEKGGGTQKPIREEERESESERSAWEQISNYSDQATQGVKEKYERAKQAASETLNTTTQTAQEKSAQAKNLAAQAKDATLEKGQQGYAVTKDTISSAAKTASEKTAPVAEKAKDYTLQAAEKAKSAGGTTASYVGEKAVQAKDVAVESGKSAAGYAAKVAADLRDKATAVGWAAAHFSAEKTVEGTKAAAHVVEGAAGYAGHKAAELASMSAGAVKGLAASAGETAKEYTAKKKEEAQRELEAKKPSQPQEAEERPSEGIGETVRQYAQKPKPSEGNPQKEGTGSIVFTAIGETVSSAGEKVKKPFKNTMGGESEGGGGKEEGKSVIGKSLTSIGEKLGDAKQREELLDNVTGNITEGGGEVLGAVGETVAEIGQNMMKPAEIVQERAHVRQAGGVLDAIGETIAEIAETTRVMVSGEDERVLRQSVVLETRVTGRAKHEEGSHGA

SEQ ID NO: 13 - DNA, 771 bp, Glycine max
  variation 242: note = a, c, g, or t
  source 1..771: mol_type = other DNA
atggccctttcttgctccaaagttttaagcttttatctctcgcccgttgtgggtggtggtgatgttccgaaaaagctcactttttcttcttttttgggtctttctaagggtgttggtggaagcaggagctcgagggtgtgtgctgcttcaaatgcacccgcaccactcgctggggtcatatttgaaccatttcaagagctcaagaaggattatcttgctgttccgattgcacacaatgtttncttggctcgtcagaactatgcagatgactctgaatctgcaatcaacgagcagatcaatgtggaatacaacgtttcctatgtgtaccacgccttgtttgcgtacttcgacagagacaacatagctctcaaaggacttgcaaagttcttcaaggaatcaagtgaagaagaaagagagcatgccgagcagcttataaagtatcagaacattcgtggtggacgagtggtgctgcacccaatcacaagtcctccctcggaatttgagcattcggaaaaaggggatgccttgtatgccatggaactagctttgtctttggagaagttgacgaatgagaaacttctgcacgttcacagtgtggcagaacgtaacaatgatcctcaaytdgcagacttcattgagagcgagttcttgtatgagcaggttaaatcgattaaaaagattgcagagtatgtggctcaactgagattagttggaaagggtcatggtgtgtggcactttgatcaaaagcttcttcacgatgaagatcatgta

SEQ ID NO: 14 - protein, 257 aa, Glycine max
  VARIANT 81: note = any amino acid
  VARIANT 208: note = any amino acid
MALSCSKVLSFYLSPVVGGGDVPKKLTFSSFLGLSKGVGGSRSSRVCAASNAPAPLAGVIFEPFQELKKDYLAVPIAHNVXLARQNYADDSESAINEQINVEYNVSYVYHALFAYFDRDNIALKGLAKFFKESSEEEREHAEQLIKYQNIRGGRVVLHPITSPPSEFEHSEKGDALYAMELALSLEKLTNEKLLHVHSVAERNNDPQXADFIESEFLYEQVKSIKKIAEYVAQLRLVGKGHGVWHFDQKLLHDEDHV

SEQ ID NO: 15 - DNA, 2421 bp, Glycine max
  source 1..2421: mol_type = other DNA
atgtctcagcaaggcgaatcatcggacccgaaatcggggaagaaggacttctcaaccgcgattctggaacgcaaaaagtcgccgaaccggcttgtcgtggatgaggctgtcaacgacgacaactccgtggtcaccatgcatccccaaaccatggaaaagcttcagcttttccgaggggacactatcctcatcaagggtaagaaacggaaggataccatttgcatagctcttgctgatgagaactgtgaggagccgaagatcaggatgaataaagttgtgaggtcaaatttgcgggttcggcttggagatgttgtgtctgtgcaccagtgcccggatgttaaatatgggaaacgtgtgcacattctgcctattgatgataccattgaaggtgtcactggcaatctgtttgatgctttcttgaaaccctatttcttggaagcttaccgtcctgtcagaaaaggagatctatttcttgtccggggagggatgagaagtgtagagttcaaggtggttgaaaccgatcctggagaatattgtgttgttgctccagacactgaaattttctgtgagggggagcctttgaaaagagaagatgaagagagactggatgaagtcgggtatgatgatgtgggtggtgtcaggaaacaaatggctcagattcgtgagttggttgagcttccattgaggcatccacagctttttaaatcaattggtgtgaaaccacccaaaggaattttactttatggacccccaggttctgggaagacattaatagcaagagctgtagctaatgaaactggagcttttttcttttgtattaacggaccagagattatgtccaaacttgctggagagagtgaaagcaatctcaggaaagcgtttgaagaagcagaaaagaatgcaccatccattatcttcattgatgaaattgattccattgcccccaagcgggagaagacacatggtgaagttgaaaggaggattgtttcacaacttttgactcttatggatggattaaaatctcgtgctcatgttattgttattggagctactaatcgtccaaatagcattgacccagcattgagaaggtttggtagatttgatagggaaattgatattggtgttcccgatgaagttgggagacttgaagtccttcgcatacatactaaaaacatgaagctatctgatgatgttgatttagagagaattgcaaaagatactcatgggtatgttggtgctgaccttgctgccctttgcactgaagctgctttgcaatgcattagggagaagatggatgtaattgacttggaggatgagactattgatgcagaagtactgaattccatggcagttacaaatgagcatttccagactgcccttggaacaagcaacccatcagctttacgtgaaactgttgttgaagtgcctaatgtcagctgggaagacattggaggccttgaaaatgtcaagcgtgaactgcaagagactgtacaatatccagttgagcacccagaaaagtttgagaagtttggtatgtctccatctaaaggagtacttttctatggtcctccaggatgtgggaaaactttgttagccaaagcaattgctaatgaatgtcaagctaacttcatcagtgtgaaaggtccagaattgcttacaatgtggtttggtgaaagtgaagctaatgttagagaaatttttgacaaggccagacaatcggctccatgtgtcctcttctttgatgagcttgactctattgccactcagagaggaagcagtgttggggatgctggtggtgcggctgatagagttctaaatcaacttttgactgaaatggatggcatgtctgccaaaaagactgtgttcataattggggcaactaacagacctgacatcattgacccagcacttcttcgaccaggccgtctggatcaattgatctatattcctctccctgatgaggattcccggcatcaaatctttaaagcctgcttgaggaagtcacctatcgcaaaaaatgttgatttgagagcattggctaggcacactcaagggttcagtggtgctgatattacagaaatatgccagcgagcatgcaaatatgccataagagaaaacatagagaaggacattgagcgagagaggaagagtagggaaaaccctgaggccatggatgaagataccgtcgacgacgaagttgcggagatcaaggctgctcattttgaggagtcaatgaagtttgcacggaggagtgttagtgatgctgatattcgcaagtatcaggcatttgctcagaccttacagcaatcaagagggtttggaagtgaatttaggtttccagagtctggagataggactactactggatctgacccctttgcagcttctgctggtggggctgacgaagatgatctatatagt

SEQ ID NO: 16 - protein, 807 aa, Glycine max
MSQQGESSDPKSGKKDFSTAILERKKSPNRLVVDEAVNDDNSVVTMHPQTMEKLQLFRGDTILIKGKKRKDTICIALADENCEEPKIRMNKVVRSNLRVRLGDVVSVHQCPDVKYGKRVHILPIDDTIEGVTGNLFDAFLKPYFLEAYRPVRKGDLFLVRGGMRSVEFKVVETDPGEYCVVAPDTEIFCEGEPLKREDEERLDEVGYDDVGGVRKQMAQIRELVELPLRHPQLFKSIGVKPPKGILLYGPPGSGKTLIARAVANETGAFFFCINGPEIMSKLAGESESNLRKAFEEAEKNAPSIIFIDEIDSIAPKREKTHGEVERRIVSQLLTLMDGLKSRAHVIVIGATNRPNSIDPALRRFGRFDREIDIGVPDEVGRLEVLRIHTKNMKLSDDVDLERIAKDTHGYVGADLAALCTEAALQCIREKMDVIDLEDETIDAEVLNSMAVTNEHFQTALGTSNPSALRETVVEVPNVSWEDIGGLENVKRELQETVQYPVEHPEKFEKFGMSPSKGVLFYGPPGCGKTLLAKAIANECQANFISVKGPELLTMWFGESEANVREIFDKARQSAPCVLFFDELDSIATQRGSSVGDAGGAADRVLNQLLTEMDGMSAKKTVFIIGATNRPDIIDPALLRPGRLDQLIYIPLPDEDSRHQIFKACLRKSPIAKNVDLRALARHTQGFSGADITEICQRACKYAIRENIEKDIERERKSRENPEAMDEDTVDDEVAEIKAAHFEESMKFARRSVSDADIRKYQAFAQTLQQSRGFGSEFRFPESGDRTTTGSDPFAASAGGADEDDLYS

SEQ ID NO: 17 - protein, 13 aa, Glycine max
DQSSSYDDDEYSK

SEQ ID NO: 18 - protein, 15 aa, Glycine max
KSDQSSSYDDDEYSK

SEQ ID NO: 19 - protein, 14 aa, Glycine max
SDQSSSYDDDEYSK

SEQ ID NO: 20 - protein, 9 aa, Glycine max
SYDDDEYSK

SEQ ID NO: 21 - protein, 18 aa, Glycine max
ALRALRLEDLRIPTAYIK

SEQ ID NO: 22 - protein, 18 aa, Glycine max
ALRLSGGDHVHAGTVVGK

SEQ ID NO: 23 - protein, 22 aa, Glycine max
LEGEREITLGFVDLLRDDFVEK

SEQ ID NO: 24 - protein, 14 aa, Glycine max
LRLEDLRIPTAYIK

SEQ ID NO: 25 - protein, 11 aa, Glycine max
LTYYTPDYETK

SEQ ID NO: 26 - protein, 11 aa, Glycine max
TDGLTSLDRYK

SEQ ID NO: 27 - protein, 14 aa, Glycine max
TFQGPPHGIQVERD

SEQ ID NO: 28 - protein, 15 aa, Glycine max
TFQGPPHGIQVERDK

SEQ ID NO: 29 - protein, 24 aa, Glycine max
APHTEDAIRVTQSTANFFISEARK

SEQ ID NO: 30 - protein, 23 aa, Glycine max
FVESGGARVIPLIYNESPENLNK

SEQ ID NO: 31 - protein, 7 aa, Glycine max
IWDLESK

SEQ ID NO: 32 - protein, 7 aa, Glycine max
SIVEDLK

SEQ ID NO: 33 - protein, 21 aa, Glycine max
QELDERARQGETVVPGGTGGK

SEQ ID NO: 34 - protein, 17 aa, Glycine max
SGEERAQEEAIDIDESK

SEQ ID NO: 35 - protein, 16 aa, Glycine max
AAAHVVEGAAGYAGHK

SEQ ID NO: 36 - protein, 11 aa, Glycine max
AKDYTLQAAEK

SEQ ID NO: 37 - protein, 17 aa, Glycine max
ARETHELGAHFESLADK

SEQ ID NO: 38 - protein, 15 aa, Glycine max
ATAVGWAAAHFSAEK

SEQ ID NO: 39 - protein, 17 aa, Glycine max
DTPQGSIEALQAGERVK

SEQ ID NO: 40 - protein, 9 aa, Glycine max
DYTLQAAEK

SEQ ID NO: 41 - protein, 10 aa, Glycine max
EEAQRELEAK

SEQ ID NO: 42 - protein, 21 aa, Glycine max
EGTGSIVFTAIGETVSSAGEK

SEQ ID NO: 43 - protein, 14 aa, Glycine max
ERGRESGGQVVAEK

SEQ ID NO: 44 - protein, 11 aa, Glycine max
GLAASAGETAK

SEQ ID NO: 45 - protein, 9 aa, Glycine max
GQQGYAVTK

SEQ ID NO: 46 - protein, 26 aa, Glycine max
KPSQPQEAEERPSEGIGETVRQYAQK

SEQ ID NO: 47 - protein, 8 aa, Glycine max
PAEIVQER

SEQ ID NO: 48 - protein, 15 aa, Glycine max
PQGSIEALQAGERVK

SEQ ID NO: 49 - protein, 17 aa, Glycine max
QAASETLNTTTQTAQEK

SEQ ID NO: 50 - protein, 13 aa, Glycine max
SAGGTTASYVGEK

SEQ ID NO: 51 - protein, 8 aa, Glycine max
SLTSIGEK

SEQ ID NO: 52 - protein, 16 aa, Glycine max
TPQGSIEALQAGERVK

SEQ ID NO: 53 - protein, 13 aa, Glycine max
VTDHAAANVVGNK

SEQ ID NO: 54 - protein, 22 aa, Glycine max
AASNAPAPLAGVIFEPFQELKK

SEQ ID NO: 55 - protein, 21 aa, Glycine max
ASNAPAPLAGVIFEPFQELKK

SEQ ID NO: 56 - protein, 18 aa, Glycine max
FFKESSEEEREHAEQLIK

SEQ ID NO: 57 - protein, 10 aa, Glycine max
GHGVWHFDQK

SEQ ID NO: 58 - protein, 13 aa, Glycine max
IAEYVAQLRLVGK

SEQ ID NO: 59 - protein, 14 aa, Glycine max
KIAEYVAQLRLVGK

SEQ ID NO: 60 - protein, 8 aa, Glycine max
LLHDEDHV

SEQ ID NO: 61 - protein, 20 aa, Glycine max
SNAPAPLAGVIFEPFQELKK

SEQ ID NO: 62 - protein, 10 aa, Glycine max
AGESESNLRK

SEQ ID NO: 63 - protein, 13 aa, Glycine max
GESEANVREIFDK

SEQ ID NO: 64 - protein, 12 aa, Glycine max
GILLYGPPGSGK

SEQ ID NO: 65 - protein, 11 aa, Glycine max
LAGESESNLRK

SEQ ID NO: 66 - protein, 12 aa, Glycine max
LSDDVDLERIAK

SEQ ID NO: 67 - protein, 16 aa, Glycine max
RELQETVQYPVEHPEK

SEQ ID NO: 68 - protein, 15 aa, Glycine max
TVFIIGATNRPDIID

SEQ ID NO: 69 - DNA, 2448 bp, Glycine max
  source 1..2448: mol_type = other DNA
atgaggaagtggacggttgcttccgctctccttcttctctctctcctctttctctttgcagatcaaggtcgaaagtttcaggcgaatgccgagggagattccgacgagcttgtagatccacccaaggtggaggacaagatcggtgctgtgcctcatggcctttcaaccgattcagatgtcgtcaagagggaggcggagtcgatctcgaagagatctctccggagcaacgcggagaagtttgagttccaagcggaagtgtcgcggcttatggatatcatcatcaattctctctacagtaacaaggatatcttcctcagagagttgatttccaatgcttctgatgctttggacaagattaggttcctctccctcacggataaggaggtgttgggagaaggtgataacaccaagcttgatattcagattaagttggacaaggaaaagaaaattctctcaattcgagacagaggtatcggtatgacgaaggaggatttgataaagaatttggggacaatagcgaaatctggaacttctgcatttgttgagaagatgcaaacaagtggagatctcaatctgattgggcagtttggagtcggcttctactctgtttatcttgtggccgactatgtcgaagttattagcaagaataacgatgataagcagtatgtatgggaatcaaaagctgatggagcattcgctatttcagaagatacatggaatgaaccacttggacgaggaactgagattagattacacctcaaggaagaggctggggaatacttggaggagtctaaactgaaagaattggtgaagagatactctgaatttattaacttccctatctatatttgggcaagcaaagaagttgatgtggaggttcccgctgatgaagatgattccagtgatgaagaggattcatctgaaagcagctccaaggaggaaagcgaagatgaagatgctgataaaagtgaagatgaagaaaaaaagcctaagacaaagacagtgaaggaaacaacttatgagtgggaacttttaaatgatgtgaaagctatatggctgaggaatccaaaagaggttacagaagaagaatacaccaaattctaccactctcttgccaaggatttcagtgatgagaaacctttagcatggagtcatttcactgctgagggtgatgttgagttcaaggcagtcctgtttgtaccacctaaggcacctcaagatttatatgagagctattataacgcaaacaaatcgaacctgaagttgtatgttagacgtgtgttcatttcagatgaatttaatgaactgttgcccaagtacctaaactttttgctgggtcttgttgattctgacactttgccacttaatgtatcacgagaaatgcttcaacaacatagtagtctgaagacaatcaagaagaaacttattaggaaagcccttgatatgatccgcaggattgctgatgaggatcctgacgagtcaactgacaaagaaaagaaagatgcatcctctgacaatgatgagaagaaaggtcaatattctaagttctggaatgagtttggtaaatccattaaacttggtatcatcgaggatgccaccaacagaaatcgtttggcaaaactgctcagatttgagagcaccaagtctgagggtaaattgacatctctggatcagtacatatctagaatgaaaactgggcagaaggatatcttttacataactggaaccagcaaagaacaactggaaaactctcccttccttgagcggcttaagaagaaaaattttgaggttattttcttcacggatccagttgatgagtacttgatgcaatacctgatggattatgaagacaagaagttccaaaatgtgtccaaggagggtttgaaacttgggaaagattcaaaagacaaggaactgaaggaatcttttaaggatcttaccaagtggtggaaaactgctctttccaaagacaatgttgatgatgtgaagatttccaaccgattagataacactccttgtgtagtggtgacatcaaaatttggttggagtgctaacatggagagaatcatgcagtctcaaactttgtcagatgctagcaagcaagcatacatgcgcggcaagagggttcttgagatcaatcctaggcaccctattatcaaggagctccgagagagagtagtaaagaaccctgaggatgagggtgtgaagcatacagcacagctgatgtaccagactgcactctttgaaagcggcttcctgcttgatgatcccaaggattttgcttcccggatttatgattcagtgaagactagcctggacatcagtcctgaagcaacagttgaggaggaagatgacactgaagtagaggctgaaagtgacgcgaaagaagattcaaagcctgaaactgatgctgttaacgatgataatgatgtcaaggacgagttgtag

SEQ ID NO: 70 - protein, 815 aa, Glycine max
MRKWTVASALLLLSLLFLFADQGRKFQANAEGDSDELVDPPKVEDKIGAVPHGLSTDSDVVKREAESISKRSLRSNAEKFEFQAEVSRLMDIIINSLYSNKDIFLRELISNASDALDKIRFLSLTDKEVLGEGDNTKLDIQIKLDKEKKILSIRDRGIGMTKEDLIKNLGTIAKSGTSAFVEKMQTSGDLNLIGQFGVGFYSVYLVADYVEVISKNNDDKQYVWESKADGAFAISEDTWNEPLGRGTEIRLHLKEEAGEYLEESKLKELVKRYSEFINFPIYIWASKEVDVEVPADEDDSSDEEDSSESSSKEESEDEDADKSEDEEKKPKTKTVKETTYEWELLNDVKAIWLRNPKEVTEEEYTKFYHSLAKDFSDEKPLAWSHFTAEGDVEFKAVLFVPPKAPQDLYESYYNANKSNLKLYVRRVFISDEFNELLPKYLNFLLGLVDSDTLPLNVSREMLQQHSSLKTIKKKLIRKALDMIRRIADEDPDESTDKEKKDASSDNDEKKGQYSKFWNEFGKSIKLGIIEDATNRNRLAKLLRFESTKSEGKLTSLDQYISRMKTGQKDIFYITGTSKEQLENSPFLERLKKKNFEVIFFTDPVDEYLMQYLMDYEDKKFQNVSKEGLKLGKDSKDKELKESFKDLTKWWKTALSKDNVDDVKISNRLDNTPCVVVTSKFGWSANMERIMQSQTLSDASKQAYMRGKRVLEINPRHPIIKELRERVVKNPEDEGVKHTAQLMYQTALFESGFLLDDPKDFASRIYDSVKTSLDISPEATVEEEDDTEVEAESDAKEDSKPETDAVNDDNDVKDEL

SEQ ID NO: 71 - protein, 14 aa, Glycine max
APQDLYESYYNANK

SEQ ID NO: 72 - protein, 8 aa, Glycine max
AVLFVPPK

SEQ ID NO: 73 - protein, 11 aa, Glycine max
DFASRIYDSVK

SEQ ID NO: 74 - protein, 17 aa, Glycine max
DIFLRELISNASDALDK

SEQ ID NO: 75 - protein, 10 aa, Glycine max
DIFYITGTSK

SEQ ID NO: 76 - protein, 7 aa, Glycine max
DNVDDVK

SEQ ID NO: 77 - protein, 17 aa, Glycine max
EDSKPETDAVNDDNDVK

SEQ ID NO: 78 - protein, 11 aa, Glycine max
EEAGEYLEESK

SEQ ID NO: 79 - protein, 13 aa, Glycine max
EQLENSPFLERLK

SEQ ID NO: 80 - protein, 10 aa, Glycine max
EVLGEGDNTK

SEQ ID NO: 81 - protein, 9 aa, Glycine max
EVTEEEYTK

SEQ ID NO: 82 - protein, 7 aa, Glycine max
FYHSLAK

SEQ ID NO: 83 - protein, 16 aa, Glycine max
IGAVPHGLSTDSDVVK

SEQ ID NO: 84 - protein, 15 aa, Glycine max
LGIIEDATNRNRLAK

SEQ ID NO: 85 - protein, 8 aa, Glycine max
NPEDEGVK

SEQ ID NO: 86 - protein, 9 aa, Glycine max
SGTSAFVEK

SEQ ID NO: 87 - protein, 26 aa, Glycine max
TSLDISPEATVEEEDDTEVEAESDAK

SEQ ID NO: 88 - DNA, 2442 bp, Glycine max
  source 1..2442: mol_type = other DNA
atgaggaagtggacggttgcttccgctctccttcttctctctctcctctttctctttgcagatcaaggtcgaaagtttcaggcgaatgccgagggagattccgacgagcttgtagatcctccgaaggtggaggacaagatcggtgctgtgcctcatggcctttccaccgattcagatgtcgtcaagagggagtcggagtcgatctcgaagagatctctccggagcaatgcggagaagtttgagttccaagcggaagtgtcacggcttatggatattatcatcaattctctctacagtaacaaggatatcttcctcagagagttgatttccaatgcttctgatgctttggacaagattaggttcctttccctcacggataaggacgttttgggagaaggtgataacaccaagcttgatattcagattaagttggacaaggaaaagaaaattctctcaattcgagacagaggtattggtatgacgaaggaggatttgataaagaatttggggacaatagcaaaatctggaacttctgcatttgttgagaagatgcaaacaagtggagatctcaatctgattgggcagtttggagtcggcttctactctgtttatctcgtggctgactatgtcgaagtcattagcaagaataatgatgataaacagtatgtatgggaatcaaaagctgatggagcatttgcaatttcagaagatacatggaatgaacctctagggcgtggaactgagattagattacacctcaaggaagaggctggggagtacttgcaggagtctaaactgaaagaattggtgaagagatactctgaatttattaacttccctatctatatttgggcaagcaaagaggttgatgtggaggttcccgctgatgaagatgattccagtgatgaagaggattcatctgaaagcagctccaaggaggaaagcgaagatgaagatgctgataaaagtgaagacgaagaaaaaaagcccaagacaaagacggtgaaggaaacaacttatgaatgggaacttttaaatgatgtgaaagctatatggctgaggaatccaaaggaggttacagaagaagaatacaccaaattctaccactctcttgccaaggatttcagtgatgagaaacctttagcatggagtcatttcactgctgagggcgatgtcgagttcaaggcagtcctgtttgtaccacctaaggcacctcaagatttatacgagagctattataacgcaaacaaatccaacctgaagttgtatgttagacgtgtgttcatttcagatgaatttaatgaactgttgcctaagtacctaaactttttgctgggtcttgttgattctgacacattgccacttaatgtatcgcgagaaatgcttcagcaacatagtagtctaaagacaatcaagaagaaacttattaggaaagcccttgatatgatccgtaggattgctgatgaggatcctgatgagtccactgacaaagaaaagaaagatacgtcatctgacaataatgagaagaaaggtcaatattccaagttctggaatgagtttggtaaatccattaaacttggtatcatcgaggatgccaccaacagaaatcgtttggcaaaactgctcagatttgagagcaccaagtctgagggtaaattgacatctctggatcagtacatatctagaatgaaagctgggcagaaggatatcttttacataactggaaccagcaaagaacaactggaaaactctccattccttgagcggcttaagaagaaaaattttgaggttattttcttcacggatccagttgatgaatacttgatgcaatacctgatggattatgaagacaagaagttccaaaatgtgtccaaggagggtttgaaactcgggaaagattcaaaagacaaggaactgaaggaatccttcaaggaccttaccaagtggtggaaaactgctctttccaaagacaatgttgatgatgtgaagatttccaaccgattggacaacactccttgtgtagtggtaacatcaaaatttggttggagtgctaacatggagagaatcatgcagtctcaaactttgtctgatgctagcaagcaagcatacatgcgcggcaagagggttcttgaggtcaatcctaggcaccctattatcaaggagctccgagagagagtagtaaagaaccctgaggatgagggtgtgaaacatacagcacagttgatgtaccagaccgcactcttcgaaagtggcttcctgcttgatgatcccaaggattttgcttcccgtatttatgattcagttaagaccagcttggacatcagtcctgaagcaacagttgaggaggaagatgacaccgaagtagaggctgaaagtgattcaaagcctgaagctgatgcggttcatgatggtgatgttgatgatgccaaggacgagttgtag

SEQ ID NO: 89 - protein, 813 aa, Glycine max
MRKWTVASALLLLSLLFLFADQGRKFQANAEGDSDELVDPPKVEDKIGAVPHGLSTDSDVVKRESESISKRSLRSNAEKFEFQAEVSRLMDIIINSLYSNKDIFLRELISNASDALDKIRFLSLTDKDVLGEGDNTKLDIQIKLDKEKKILSIRDRGIGMTKEDLIKNLGTIAKSGTSAFVEKMQTSGDLNLIGQFGVGFYSVYLVADYVEVISKNNDDKQYVWESKADGAFAISEDTWNEPLGRGTEIRLHLKEEAGEYLQESKLKELVKRYSEFINFPIYIWASKEVDVEVPADEDDSSDEEDSSESSSKEESEDEDADKSEDEEKKPKTKTVKETTYEWELLNDVKAIWLRNPKEVTEEEYTKFYHSLAKDFSDEKPLAWSHFTAEGDVEFKAVLFVPPKAPQDLYESYYNANKSNLKLYVRRVFISDEFNELLPKYLNFLLGLVDSDTLPLNVSREMLQQHSSLKTIKKKLIRKALDMIRRIADEDPDESTDKEKKDTSSDNNEKKGQYSKFWNEFGKSIKLGIIEDATNRNRLAKLLRFESTKSEGKLTSLDQYISRMKAGQKDIFYITGTSKEQLENSPFLERLKKKNFEVIFFTDPVDEYLMQYLMDYEDKKFQNVSKEGLKLGKDSKDKELKESFKDLTKWWKTALSKDNVDDVKISNRLDNTPCVVVTSKFGWSANMERIMQSQTLSDASKQAYMRGKRVLEVNPRHPIIKELRERVVKNPEDEGVKHTAQLMYQTALFESGFLLDDPKDFASRIYDSVKTSLDISPEATVEEEDDTEVEAESDSKPEADAVHDGDVDDAKDEL

SEQ ID NO: 90 - protein, 14 aa, Glycine max
APQDLYESYYNANK

SEQ ID NO: 91 - protein, 8 aa, Glycine max
AVLFVPPK

SEQ ID NO: 92 - protein, 11 aa, Glycine max
DFASRIYDSVK

SEQ ID NO: 93 - protein, 17 aa, Glycine max
DIFLRELISNASDALDK

SEQ ID NO: 94 - protein, 10 aa, Glycine max
DIFYITGTSK

SEQ ID NO: 95 - protein, 7 aa, Glycine max
DNVDDVK

SEQ ID NO: 96 - protein, 10 aa, Glycine max
DVLGEGDNTK

SEQ ID NO: 97 - protein, 11 aa, Glycine max
EEAGEYLQESK

SEQ ID NO: 98 - protein, 13 aa, Glycine max
EQLENSPFLERLK

SEQ ID NO: 99 - protein, 9 aa, Glycine max
EVTEEEYTK

SEQ ID NO: 100 - protein, 7 aa, Glycine max
FYHSLAK

SEQ ID NO: 101 - protein, 16 aa, Glycine max
IGAVPHGLSTDSDVVK

SEQ ID NO: 102 - protein, 15 aa, Glycine max
LGIIEDATNRNRLAK

SEQ ID NO: 103 - protein, 8 aa, Glycine max
NPEDEGVK

SEQ ID NO: 104 - protein, 9 aa, Glycine max
SGTSAFVEK

SEQ ID NO: 105 - DNA, 2103 bp, Glycine max
  source 1..2103: mol_type = other DNA
atggcttcggagactgagacgttcgcttttcaggccgagatcaaccagctcctcagtctaataatcaacaccttctacagcaacaaggagatctttcttcgtgagctcatcagcaatgcctctgacgctttggacaagattcgatttgagagtcttacagacaagagcaagcttgatgctcagccagagttgttcattcacattattcctgacaagaccaacaacacattgtctatcattgacagtggtattggcatgactaaggctgatttggtgaataacctcggtactattgcaaggtctgggaccaaggaattcatggaagccctggctgctggtgctgacgtgagcatgattgggcagttcggtgttggtttctactcggcttatcttgtagctgataaggttattgttaccacaaagcacaatgatgatgaacagtatgtctgggagtcccatgccggtggctcatttactgtgacaagagatacatctggtgagaaccttggtaggggaacaaagataactttgtttcttaaggaagatcaacttgagtatcttgaggaacgccgtctgaaggatttgataaagaagcattctgaattcattagctatccaatttctctctggattgagaagactactgaaaaggagatttctgatgatgaggatgaggaagagaagaaggatgaggagggtaaagttgaagatgtggatgaagataaggagaaggaggagaagaaaaagaagaccattaaggaggtatctcacgagtggtcattggtgaacaagcagaagcctatttggatgaggaaacctgaagagattacgaaagaagaatatgctgctttctacaagagtcttaccaatgattgggaagagcatttggctgttaagcacttttctgttgagggtcagctggagtttaaggctgtcctctttatccccaagagggcaccttttgatctctttgacaccaggaagaagcctaacaacatcaaactgtatgtccgccgtgtcttcatcatggacaactgtgaggagttgatgcctgaatatcttagctttgttaagggtattgtggattctgaggatcttcctctcaacatttctagagaaatgctgcagcagaacaagatcctgaaagtcatcaggaagaacttggtcaagaagtgcattgagatgttctttgaaattgctgaaaacaaggaagactataacaagttttatgaagccttctctaagaacctgaaacttggtattcatgaggattctcagaacaagacaaagctagctgaattgctcaggtatcactccactaagagtggtgatgagatgaccagcctcaaggactatgttaccaggatgaaggaaggacagaatgacatctactacattactggtgaaagcaagaaagctgtcgagaattcccccttccttgaaaagcttaagaagaagggatacgaggttctctacatggttgatgctattgatgagtatgctgttggccagcttaaggaatttgagggaaagaagttggtctctgctaccaaggaaggcctaaaacttgatgagagcgaagacgagaagaaaaagaaggaagaactaaaggataaatttgagggtctttgccatgtgatcaaggatgtgttgggtgacaaggtggagaaagttgtggtgtctgatcgtgttgtggattctccttgctgtctggtgacaggtgaatatgggtggacagccaacatggaaaggattatgaaagcacaggcattgagggacagcagcatggctggatacatgtcaagcaagaagacgatggagattaaccctgagaaccccatcatggaggagctgaggaagcgagcagatgctgataagaatgacaagtctgtgaaggatctcgtgctcttgctctttgagactgctcttctaacttctgggttcagccttgatgatcccaacactttcggtaacaggattcacaggatgctgaagcttggactgagcattgatgaagatgctggtgaagcagatgctgacatgcctcctcttgaggacgctgatgcagatgctgagggtagcaagatggaagaagttgattaa

SEQ ID NO: 106 - protein, 700 aa, Glycine max
MASETETFAFQAEINQLLSLIINTFYSNKEIFLRELISNASDALDKIRFESLTDKSKLDAQPELFIHIIPDKTNNTLSIIDSGIGMTKADLVNNLGTIARSGTKEFMEALAAGADVSMIGQFGVGFYSAYLVADKVIVTTKHNDDEQYVWESHAGGSFTVTRDTSGENLGRGTKITLFLKEDQLEYLEERRLKDLIKKHSEFISYPISLWIEKTTEKEISDDEDEEEKKDEEGKVEDVDEDKEKEEKKKKTIKEVSHEWSLVNKQKPIWMRKPEEITKEEYAAFYKSLTNDWEEHLAVKHFSVEGQLEFKAVLFIPKRAPFDLFDTRKKPNNIKLYVRRVFIMDNCEELMPEYLSFVKGIVDSEDLPLNISREMLQQNKILKVIRKNLVKKCIEMFFEIAENKEDYNKFYEAFSKNLKLGIHEDSQNKTKLAELLRYHSTKSGDEMTSLKDYVTRMKEGQNDIYYITGESKKAVENSPFLEKLKKKGYEVLYMVDAIDEYAVGQLKEFEGKKLVSATKEGLKLDESEDEKKKKEELKDKFEGLCHVIKDVLGDKVEKVVVSDRVVDSPCCLVTGEYGWTANMERIMKAQALRDSSMAGYMSSKKTMEINPENPIMEELRKRADADKNDKSVKDLVLLLFETALLTSGFSLDDPNTFGNRIHRMLKLGLSIDEDAGEADADMPPLEDADADAEGSKMEEVD

SEQ ID NO: 107 - protein, 16 aa, Glycine max
ADLVNNLGTIARSGTK

SEQ ID NO: 108 - protein, 10 aa, Glycine max
AVENSPFLEK

SEQ ID NO: 109 - protein, 13 aa, Glycine max
EDQLEYLEERRLK

SEQ ID NO: 110 - protein, 8 aa, Glycine max
EEYAAFYK

SEQ ID NO: 111 - protein, 14 aa, Glycine max
EGQNDIYYITGESK

SEQ ID NO: 112 - protein, 17 aa, Glycine max
EIFLRELISNASDALDK

SEQ ID NO: 113 - protein, 7 aa, Glycine max
FYEAFSK

SEQ ID NO: 114 - protein, 11 aa, Glycine max
HFSVEGQLEFK

SEQ ID NO: 115 - protein, 9 aa, Glycine max
IRFESLTDK

SEQ ID NO: 116 - protein, 11 aa, Glycine max
LAELLRYHSTK

SEQ ID NO: 117 - protein, 8 aa, Glycine max
LDESEDEK

SEQ ID NO: 118 - protein, 10 aa, Glycine max
LGIHEDSQNK

SEQ ID NO: 119 - protein, 11 aa, Glycine max
RAPFDLFDTRK

SEQ ID NO: 120 - protein, 8 aa, Glycine max
VEDVDEDK

SEQ ID NO: 121 - DNA, 2109 bp, Glycine max
  source 1..2109: mol_type = other DNA
atggcggagacagagacgttcgcattccaagcagagatcaaccagctcctcagtctcatcatcaacaccttctactccaacaaggagattttccttcgtgaactcatcagcaacgcctccgatgctttggacaagattcgatttgagagtttgacagataagagcaagctcgatgctcagccagagttgttcattcatattattccagacaagactaacaatacactgaccattgttgacagtggtattggcatgaccaaagctgatttggtgaacaacttgggtacaattgctagatctggtaccaaggaattcatggaagccttggctgctggtgctgatgttagtatgattggacagtttggtgtggggttctactctgcatatcttgttgctgagaaggtcattgttacaaccaaacacaatgatgatgaacagtatgtttgggagtctcaagctggaggttctttcactgtcaccagggataacactggtgaggttcttggcaggggtactaagatcactctctacctcaaggaagatcagcttgagtaccttgaggagcgtcgcttgaaggatctgatcaagaagcattctgagttcattagctacccaatttctctttggattgagaagactactgagaaggagatctctgacgatgaggatgaggaagagaagaaggatgaggagggtaaggtggaggatgttgacgaagagaaggagaaggaagaaaaaaagaaaaagaagatcaaggaagtgtcacatgaatggtccttggtgaacaagcagaagcccatttggatgaggaagcctgaggagatcacaaaagaggagtattctgctttctacaagagtcttaccaatgactgggaagagcatttggctgtcaagcacttctctgttgagggtcagttggagtttaaggctatactctttgttcccaagagggcaccttttgacctctttgacacaaggaagaagcctaacaacattaagctctatgtccgccgtgtctttattatggacaactgtgaggagttgattcctgagtatttgggctttgtcaagggtattgttgattctgaagatctcccactcaacatttcaagagaaatgttgcaacagaacaagatcttgaaggtcatccgtaagaacttggtcaagaagtgccttgagctcttctttgaaattgcagagaacaaggaggattacaacaagttctatgaggctttctctaagaacttaaagcttggtatccatgaggattcccagaacaagggaaagattgctgaattgctcaggtaccactccaccaagagtggtgatgagctgacaagcctcaaggattatgtgaccaggatgaaggaaggtcagagtgacatctactacatcaccggtgaaagcaagaaggctgttgagaactccccattcttggagaagttgaagaagaaagggtatgaggttcttttcatggttgatgccattgatgaatatgctgttggccagctcaaggaatttgagggcaagaagcttgtctctgccaccaaggagggtctcaaacttgacgagagtgaagatgagaagaagaagcaagaggagttgaaggagaagtttgacaacctatgcaaggtgatcaaggatgttttgggtgacaaggttgagaaggttgtagtctctgaccgtgttgttgattcaccatgctgtctggtgactggagaatacggttggactgcaaacatggaaaggatcatgaaagctcaagccctaagggacaacagcatggcaggatacatgtcaagcaagaagacaatggagatcaaccctgagaacccaatcatggaggagctcaggaagcgtgcggatgctgacaagaacgacaaatccgtgaaggaccttgttctgttgctgttcgagactgctcttctcacttctggtttcagccttgacgaacccaacacttttggaaacaggatccacagaatgttgaagcttggactgagcatcgacgaggatgcagctgaagctgatgctgatgctgatatgcccccactggaggaggaggctgaagctgatgctgagggcagcaaaatggaggaggttgattaa

SEQ ID NO: 122 - protein, 702 aa, Glycine max
MAETETFAFQAEINQLLSLIINTFYSNKEIFLRELISNASDALDKIRFESLTDKSKLDAQPELFIHIIPDKTNNTLTIVDSGIGMTKADLVNNLGTIARSGTKEFMEALAAGADVSMIGQFGVGFYSAYLVAEKVIVTTKHNDDEQYVWESQAGGSFTVTRDNTGEVLGRGTKITLYLKEDQLEYLEERRLKDLIKKHSEFISYPISLWIEKTTEKEISDDEDEEEKKDEEGKVEDVDEEKEKEEKKKKKIKEVSHEWSLVNKQKPIWMRKPEEITKEEYSAFYKSLTNDWEEHLAVKHFSVEGQLEFKAILFVPKRAPFDLFDTRKKPNNIKLYVRRVFIMDNCEELIPEYLGFVKGIVDSEDLPLNISREMLQQNKILKVIRKNLVKKCLELFFEIAENKEDYNKFYEAFSKNLKLGIHEDSQNKGKIAELLRYHSTKSGDELTSLKDYVTRMKEGQSDIYYITGESKKAVENSPFLEKLKKKGYEVLFMVDAIDEYAVGQLKEFEGKKLVSATKEGLKLDESEDEKKKQEELKEKFDNLCKVIKDVLGDKVEKVVVSDRVVDSPCCLVTGEYGWTANMERIMKAQALRDNSMAGYMSSKKTMEINPENPIMEELRKRADADKNDKSVKDLVLLLFETALLTSGFSLDEPNTFGNRIHRMLKLGLSIDEDAAEADADADMPPLEEEAEADAEGSKMEEVD

SEQ ID NO: 123 - protein, 16 aa, Glycine max
ADLVNNLGTIARSGTK

SEQ ID NO: 124 - protein, 10 aa, Glycine max
AVENSPFLEK

SEQ ID NO: 125 - protein, 13 aa, Glycine max
EDQLEYLEERRLK

SEQ ID NO: 126 - protein, 8 aa, Glycine max
EEYSAFYK

SEQ ID NO: 127 - protein, 14 aa, Glycine max
EGQSDIYYITGESK

SEQ ID NO: 128 - protein, 17 aa, Glycine max
EIFLRELISNASDALDK

SEQ ID NO: 129 - protein, 7 aa, Glycine max
FYEAFSK

SEQ ID NO: 130 - protein, 11 aa, Glycine max
HFSVEGQLEFK

SEQ ID NO: 131 - protein, 11 aa, Glycine max
IAELLRYHSTK

SEQ ID NO: 132 - protein, 9 aa, Glycine max
IRFESLTDK

SEQ ID NO: 133 - protein, 8 aa, Glycine max
LDESEDEK

SEQ ID NO: 134 - protein, 10 aa, Glycine max
LGIHEDSQNK

SEQ ID NO: 135 - protein, 11 aa, Glycine max
RAPFDLFDTRK

SEQ ID NO: 136 - protein, 9 aa, Glycine max
SGDELTSLK

SEQ ID NO: 137 - protein, 8 aa, Glycine max
VEDVDEEK

SEQ ID NO: 138 - DNA, 2109 bp, Glycine max
  source 1..2109: mol_type = other DNA
atggcttcggagactgagacgtttgcttttcaggccgagatcaaccagctcctcagtctaatcatcaacaccttctacagcaacaaggagatctttcttcgtgagctcatcagcaatgcctctgatgctttggacaagattcgatttgagagtcttacagacaagagcaagcttgatgctcagccagagttgttcattcacattattcctgacaagaccaacaacacattgtctatcattgacagtggtattggcatgactaaggccgatttggtgaataacctcggtactattgcaaggtctgggaccaaggaattcatggaagccctggctgctggtgctgatgtgagcatgattggacagtttggtgttggtttctactcggcttatcttgtagctgacaaggttattgttaccacaaagcacaatgatgatgagcagtatgtctgggagtcccatgctggtggctcatttactgtgacaagggatacatctggtgagaaccttggtaggggaacaaagataactttgtttctcaaggaagatcaacttgagtatcttgaggaacgccgtctgaaggatttgataaagaagcattctgaattcattagctatccaatttctctttggattgagaagaccactgaaaaggagatttctgatgatgaggacgaggaagagaagaaggatgaggagggtaaagttgaagatgtggatgaagataaggagaaggaggagaagaaaaagaagaccattaaggaggtatctcatgagtggtcattggtgaacaagcagaagcctatttggatgaggaaacctgaagagatcacaaaagaagaatatgctgctttctacaagagtcttaccaatgattgggaagagcatttggctgttaagcacttttctgttgagggtcagctggagtttaaggctgtcctctttatccccaagagggcaccttttgatctttttgacactaagaagaagcctaacaacatcaaactgtatgtccgccgtgttttcatcatggacaactgtgaggagttgatgccagaatatcttagctttgttaagggtattgtggattctgaggatcttcctctcaacatttctagagaaatgctgcagcagaacaagatcctgaaagtcatcaggaagaacttggtcaagaagtgcattgagatgttctttgaaattgctgagaacaaggaagactataacaagttttatgaagccttctctaagaatctgaaacttggtattcatgaggattctcagaacaagacaaaacttgctgaattgctcaggtatcactccaccaagagtggtgatgagatgaccagcctcaaggactatgttaccaggatgaaggaagggcagaatgacatctactacattactggtgaaagcaagaaagctgttgagaattcccctttccttgaaaagcttaagaagaaggggttcgaggttctctacatggttgatgctattgacgagtatgctgttggtcagcttaaggaatttgagggaaagaagttggtctctgctactaaggaaggcctgaaacttgacgagagcgaagacgaaaagaaaaagaaggaagaattgaaggagaaatttgagggtctctgccatgtgatcaaggatgtgttgggtgacaaggtggagaaagttgtggtgtctgatcgtgttgtggattctccttgctgtctcgtgactggtgaatatgggtggacagccaacatggaaaggattatgaaagcacaggcattgagggacagcagcatggctggatacatgtcgagcaagaagaccatggagattaaccctgagaaccccatcatggatgaactgaggaagcgagctgatgctgacaagaatgacaaatctgtgaaggatctcgttctcctactctttgagactgctcttctaacttctgggttcagccttgatgatcccaacactttcggtaacaggattcacaggatgctgaagcttggactgagcattgatgaagatgctggtgaagcagatgctgacatgcctcctctcgaggacgccgatgctgatgccgatgctgagggtagcaagatggaagaagttgattaa

SEQ ID NO: 139 - protein, 702 aa, Glycine max
MASETETFAFQAEINQLLSLIINTFYSNKEIFLRELISNASDALDKIRFESLTDKSKLDAQPELFIHIIPDKTNNTLSIIDSGIGMTKADLVNNLGTIARSGTKEFMEALAAGADVSMIGQFGVGFYSAYLVADKVIVTTKHNDDEQYVWESHAGGSFTVTRDTSGENLGRGTKITLFLKEDQLEYLEERRLKDLIKKHSEFISYPISLWIEKTTEKEISDDEDEEEKKDEEGKVEDVDEDKEKEEKKKKTIKEVSHEWSLVNKQKPIWMRKPEEITKEEYAAFYKSLTNDWEEHLAVKHFSVEGQLEFKAVLFIPKRAPFDLFDTKKKPNNIKLYVRRVFIMDNCEELMPEYLSFVKGIVDSEDLPLNISREMLQQNKILKVIRKNLVKKCIEMFFEIAENKEDYNKFYEAFSKNLKLGIHEDSQNKTKLAELLRYHSTKSGDEMTSLKDYVTRMKEGQNDIYYITGESKKAVENSPFLEKLKKKGFEVLYMVDAIDEYAVGQLKEFEGKKLVSATKEGLKLDESEDEKKKKEELKEKFEGLCHVIKDVLGDKVEKVVVSDRVVDSPCCLVTGEYGWTANMERIMKAQALRDSSMAGYMSSKKTMEINPENPIMDELRKRADADKNDKSVKDLVLLLFETALLTSGFSLDDPNTFGNRIHRMLKLGLSIDEDAGEADADMPPLEDADADADAEGSKMEEVD

SEQ ID NO: 140 - protein, 16 aa, Glycine max
ADLVNNLGTIARSGTK

SEQ ID NO: 141 - protein, 10 aa, Glycine max
AVENSPFLEK

SEQ ID NO: 142 - protein, 13 aa, Glycine max
EDQLEYLEERRLK

SEQ ID NO: 143 - protein, 8 aa, Glycine max
EEYAAFYK

SEQ ID NO: 144 - protein, 14 aa, Glycine max
EGQNDIYYITGESK

SEQ ID NO: 145 - protein, 17 aa, Glycine max
EIFLRELISNASDALDK

SEQ ID NO: 146 - protein, 7 aa, Glycine max
FYEAFSK

SEQ ID NO: 147 - protein, 11 aa, Glycine max
HFSVEGQLEFK

SEQ ID NO: 148 - protein, 9 aa, Glycine max
IRFESLTDK

SEQ ID NO: 149 - protein, 11 aa, Glycine max
LAELLRYHSTK

SEQ ID NO: 150 - protein, 8 aa, Glycine max
LDESEDEK

SEQ ID NO: 151 - protein, 10 aa, Glycine max
LGIHEDSQNK

SEQ ID NO: 152 - protein, 10 aa, Glycine max
RAPFDLFDTK

SEQ ID NO: 153 - protein, 8 aa, Glycine max
VEDVDEDK

SEQ ID NO: 154 - DNA, 2109 bp, Glycine max; identical to SEQ ID NO: 138
  source 1..2109: mol_type = other DNA

SEQ ID NO: 155 - protein, 839 aa, Glycine max
MKLSPREVEKLGLHNAGYLAQKRLARGLRLNYTEAVALIATQIMEFARDGEKTVAQLMCIGKHLLGRRQVLPEVQHLLNAVQVEATFPDGTKLVTVHDPISCEHGDLGQALFGSFLPVPSLDKFAENKEDNRIPGEIIYGDGSLVLNPGKNAVILKVVSNGDRPIQVGSHYHFIEVNPYLTFDRRKAYGMRLNIAAGTAVRFEPGDSKSVKLVRIGGNKVIRGGNGIADGQVNETNLREAMEAVCKRGFGHKEEEDASEGITGDPDSPFTTIIPREEYANKYGPTTGDKIRLGDTDLFAKIEKDFALYGDECVFGGGKVLRDGMGQSCGDPPAISLDTVITNAVIIDYSGIIKADIGIKDGLIVSIGKAGNPDIMDDVFFNMIIGANTEVIAGEGLIVTAGAIDCHVHYICPQLVDEAISSGITTLVGGGTGPTAGTRATTCTPAPSQMKLMLQSTDDLPLNFGFTGKGSSSKPDELHDIIKAGAMGLKLHEDWGSTPAAIDSCLTVADQYDIQINIHTDTLNEAGFVEHSIAAFKGRTIHTYHSEGAGGGHAPDIIKVCGMKNVLPSSTNPTRPLTLNTIDEHLDMLMVCHHLNREIPEDLAFACSRIREGTIAAEDILHDIGAISIISSDSQAMGRVGEVISRTWQTANKMKVQRGPLQPGESDNDNFRIKRYIAKYTINPAIANGFSQYVGSVEVGKLADLVMWKPSFFGAKPEMVIKGGVVAWADMGDPNASIPTPEPVKMRPMFGTLGKAGGALSIAFVSKAAVDQRVHALYGLNKRVEAVGNVRKLTKLDMKLNDSLPQITVDPDNYTVTADGEVLTSFATTFVPLSRNYFLF

SEQ ID NO: 156 - protein, 12 aa, Glycine max
AGGALSIAFVSK

SEQ ID NO: 157 - protein, 9 aa, Glycine max
DGLIVSIGK

SEQ ID NO: 158 - DNA, 2946 bp, Glycine max
  source 1..2946: mol_type = other DNA
atggcttcttcttccacgacaatgcggttctccgcggcctccaccggcgccgcggcggcgcttcctcgccggagctccgtcgccggattcatcggcgtcgcggccagatcctccgccaaatcgaggctccgcttcatcggaaggaacgcgaacttgagcctccggatgaggaggatgagctctttctccgtcgtgaagtgcgtctccggaagcgaggccaaggtgcaggataccgtggccaaacaacaagaagctacaacctctctcagctccttcacgccagatgcttcatctattgcctcaagtattaaataccatgcagagttcactccattgttttctccagagaattttgatctaccacaggcttttcttgcaactgcacaaagtgttcgagattctctcattataaactggaatgctacctatgattactatgagaaactgaatgttaagcaggcatattacctttcgatggaatttttgcagggtagagcattattaaatgcaataggcaatctggagctaactggtccctatgctgaggctttgagcaagctcgggcataaattagaaaatgtggcataccaggagccagatgctgcacttggaaatggtggtcttgggcgtcttgcttcatgtttcttggactctttggcaaccttgaactatccagcttggggttatggattgagatataagtatggtttatttaaacagcgaataaccaaagatgggcaggaggaagttgctgaagattggcttgagatgggcaatccctgggagattattagaaatgatgtttcatatcctgtaaagttctatggcaaggttgtttcagggtcagatggtaaaaaacattggattggaggagaagatataaaagctgttgcacatgatgttcccataccaggatataaaactaagactacaatcaacttgagactttggtctacaaaagctgcatcagaagaatttgatttatctgcttttaatgctggaaggcacactgaagcatctgaggctctagcaaatgctgaaaagatatgctatatactctaccctggggatgaaccaatagagggcaagatccttcgcctcaagcaacaatatactttatgttctgcttctcttcaagacatcattgctcgttttgagagaagatcaggagcaaatgttaattgggaagagtttcccgagaaggttgcagtgcagatgaatgatactcacccaacgttgtgcatcccagagctcatgagaattttaatagatgtaaagggcttaaattggaaggatgcttggaatattacacaaaggactgtagcatatacaaaccacactgttcttccagaggcattagagaaatggagcttggatcttatgcagaaactgcttccacgccatattgagataatagaaatgattgacgaggagctggttcggaccataattgcagaatatggcacagaaaattcagacttgctggagaaaaaattgaaggaaatgagaatactagaaaatgtcgaattgactgcagaatttgcagatatactggttaagtccaaagaggccattgatattcctagtgaggagctgcaaagttctgaacaagcagaagcagaagacgaaaaagatgatgatgaagtggaagcagttgctaaaaagaatggaacagatgaaagttccattgaggatgagaaagaagagttgcctgagccagtaccagaaccaccaaaattggttcgtatggcaaatttgtgtgttgtgggtggtcatgcagtgaatggggttgcagagatacatagtgaaattgtaaaagatgatgttttcaatgcattttataagttgtggcccgagaagttccaaaacaaaacaaatggtgtgacaccaaggagatggatcaggttctgcaatccagatctgagtaaaataataaccgagtggataggtactgaggattgggtcctaaatactggaaaattggccgagctacggaagtttgtagacaatgaggatctacaagtgcagtggagggaagcaaagaggagtaacaaagttaaagttgcagcattcatcagagaaaaaacagggtattctgtcagtcctgatgcaatgtttgacatccaggtgaagagaatccatgaatataagcgacaattaatgaacatatttggaattgtttaccgatacaagaagatgaaagaaatgagtgcggcagaaagggaagcaaattttgttcctagagtttgtatatttgggggaaaagcatttgctacatatgtgcaagccaagagaattgtgaaatttattacagatgttggggctacagtaaatcatgaccctgaaataggagatctacttaaggttgtttttgtccctgattataatgtcagtgttgccgagatgcttattccagcaagtgaattgtcacaacatatcagtactgctggaatggaggcaagtggaactagcaatatgaaatttgcaatgaatggctgcatattgattggaactttagatggggccaatgttgaaataagggaagaggttggtgcagacaacttcttcctctttggtgccaaggctcatgaaattgctgggctgaggaaagaaagagcagagggaaagtttgtgcctgatccacgatttgaagaagttaaagaatttgtcagaagtggcatttttggatcttacaactatgacgaattgatgggatccttagaaggaaatgaaggttttggtcgagcagattatttccttgtagggaaagactttcccagctacatagaatgccaagagaaggttgatgaagcataccgtgaccaaactaaatggacaagaatgtcaatattgaacacagcaggttcatacaagtttagcagtgaccgcacgattcatgaatatgcccgagagatatggaacattgaaccagtccaattgccatag

SEQ ID NO: 159 - protein, 981 aa, Glycine max
MASSSTTMRFSAASTGAAAALPRRSSVAGFIGVAARSSAKSRLRFIGRNANLSLRMRRMSSFSVVKCVSGSEAKVQDTVAKQQEATTSLSSFTPDASSIASSIKYHAEFTPLFSPENFDLPQAFLATAQSVRDSLIINWNATYDYYEKLNVKQAYYLSMEFLQGRALLNAIGNLELTGPYAEALSKLGHKLENVAYQEPDAALGNGGLGRLASCFLDSLATLNYPAWGYGLRYKYGLFKQRITKDGQEEVAEDWLEMGNPWEIIRNDVSYPVKFYGKVVSGSDGKKHWIGGEDIKAVAHDVPIPGYKTKTTINLRLWSTKAASEEFDLSAFNAGRHTEASEALANAEKICYILYPGDEPIEGKILRLKQQYTLCSASLQDIIARFERRSGANVNWEEFPEKVAVQMNDTHPTLCIPELMRILIDVKGLNWKDAWNITQRTVAYTNHTVLPEALEKWSLDLMQKLLPRHIEIIEMIDEELVRTIIAEYGTENSDLLEKKLKEMRILENVELTAEFADILVKSKEAIDIPSEELQSSEQAEAEDEKDDDEVEAVAKKNGTDESSIEDEKEELPEPVPEPPKLVRMANLCVVGGHAVNGVAEIHSEIVKDDVFNAFYKLWPEKFQNKTNGVTPRRWIRFCNPDLSKIITEWIGTEDWVLNTGKLAELRKFVDNEDLQVQWREAKRSNKVKVAAFIREKTGYSVSPDAMFDIQVKRIHEYKRQLMNIFGIVYRYKKMKEMSAAEREANFVPRVCIFGGKAFATYVQAKRIVKFITDVGATVNHDPEIGDLLKVVFVPDYNVSVAEMLIPASELSQHISTAGMEASGTSNMKFAMNGCILIGTLDGANVEIREEVGADNFFLFGAKAHEIAGLRKERAEGKFVPDPRFEEVKEFVRSGIFGSYNYDELMGSLEGNEGFGRADYFLVGKDFPSYIECQEKVDEAYRDQTKWTRMSILNTAGSYKFSSDRTIHEYAREIWNIEPVQLP

SEQ ID NO: 160 - protein, 9 aa, Glycine max
AFATYVQAK

SEQ ID NO: 161 - protein, 23 aa, Glycine max
QQEATTSLSSFTPDASSIASSIK

SEQ ID NO: 162 - DNA, 2706 bp, Glycine max
  source 1..2706: mol_type = other DNA
atggcgactgagaatcctttcaatagcattttgacgacgctcgaaaagcccggtggtgctggcgagtttggaaagtactttagcttgcctgctctcaacgatcgtagaatcgatagacttccttactctgtgaggatacttttggaatctgcaatccggaactgtgatgagtttcaagttaagagtaacgacgttgaaaaaattattgattgggagaatacctctccgaagctggtggagattccattcaagccagctagagtgcttctccaggattttactggcgtgcctgctgttgtcgatcttgcttgcatgcgagatgccatgaacaaacttggtggtgattctaataaaattaatcccttggtaccagttgatcttgtcattgatcactctgttcaagttgatgtggcaagatcagaaaatgctgtccaggcaaatatggaacttgaattccaaagaaacaaggaaagatttggtttcctcaaatggggttctaatgcattcaataatatgcttgtcgttcctcctggatcagggatagttcaccaggttaatttagaataccttggtagagttgtgttcaacacaaatggtgtgctttatcctgacagcgttgttggaactgattcacacacaactatgattgatggcctgggtgttgctggatggggagttggtggaatagaagcagaagctgcaatgcttggccaacccatgagcatggtcctaccgggtgtcgttgggttcaaattattaggcaaactacgagatggtgtcacagctactgacttggtgttgactgttacacaaatgctaagaaagcatggggttgttggcaagtttgtagagttttatggggaaggcatgagtgaactgtctttggcagatcgtgccaccatagcaaacatgtctcctgagtatggtgcaaccatgggcttctttcctgtggatcatgtcactttgcaatatttgagactgactggcagaagtgatgagactgtttctatgatagaatcgtacttacgagcaaataagatgtttgtggattacagcgagccccaagtggagagagtgtactcatcgtatttggaactcaatcttgaggatgtagaaccctgtgtctcaggtccaaaaaggcctcatgaccgtgtccctttgagagaaatgaaggtagactggcatgcctgcctcaacaataaagttggattcaagggttttgctgtaccaaaagaatctcagaataaggttgcagagttcacattccaagggacaccagcacatcttaggcacggtgatgttgttatagctgctatcaccagttgtacaaatacttcaaatcctagcgtaatgcttggagctgcattggttgcaaagaaagcatgtgaattgggattgcaggtgaagccatggattaaaacaagccttgccccaggttctggtgttgtgactaagtatttgcagaggagtggcttgcagaagtatttgaatgagctagggtttaatatagttgggtatggatgcaccacatgcattggaaattcaggggatattaatgaagctgttgcatctgcaattactgaaaatgatatagtagctgcagctgtactgtctggaaataggaactttgagggccgagttcacccattaacaagagctaattatcttgcttctcctcctcttgttgttgcctatgcccttgctggcacagtggacattgactttgacactgaacccattgggatagggaaggatggcacaaagatcttcttcagggatatttggccatccagtgaagaaatagcaaatgttgtacaatcaagtgtgctgcctgctatgtttagggacacatataatgcgatcacccaagggaatcccatgtggaataatttatctgttccaactggcactctttatgcttgggaccctacatcaacttatattcatgagccaccttattttagagatatgagcatgtctcccccaggctctcatggagtgaaagatgcttactgtttgctcaactttggagacagtatcacaactgaccacatctctccagctggcagcatacataaggacagtcctgcagccagataccttatagaacgtggggttgatagacgggacttcaactcctatggaagtcgccgtggtaatgatgaggtgatggcccgaggaacttttgccaatattcgcattgtcaacaaatttttgaatggagaagttggacctaaaactattcatattccttctggggagaagttatcagtctttgatgctgcagagaaatacaagagtgaggggcatgatatgattatcttggctggtgctgagtatgggagtggaagctctcgtgattgggctgcaaaggggccaatgctactgggcgtgaaagctgtcatagcaaaaagttttgaaaggattcaccgaagtaatttggtgggaatgggtatcattccattgtgttttaagcccggggatgatgctgattctcttggattaactggccatgaacgttacaccattgatctaccaagcaatgtgaatgaaatacgacccggccaagatgtcacagtggtgacagatgccgggaaatcatttgtatctacactgagatttgatacagaggttgaactagcttacttcaaccatggaggcatcttacaatatgtcatcagaaacatggtcaatgctaaacattga

SEQ ID NO: 163 - protein, 901 aa, Glycine max
MATENPFNSILTTLEKPGGAGEFGKYFSLPALNDRRIDRLPYSVRILLESAIRNCDEFQVKSNDVEKIIDWENTSPKLVEIPFKPARVLLQDFTGVPAVVDLACMRDAMNKLGGDSNKINPLVPVDLVIDHSVQVDVARSENAVQANMELEFQRNKERFGFLKWGSNAFNNMLVVPPGSGIVHQVNLEYLGRVVFNTNGVLYPDSVVGTDSHTTMIDGLGVAGWGVGGIEAEAAMLGQPMSMVLPGVVGFKLLGKLRDGVTATDLVLTVTQMLRKHGVVGKFVEFYGEGMSELSLADRATIANMSPEYGATMGFFPVDHVTLQYLRLTGRSDETVSMIESYLRANKMFVDYSEPQVERVYSSYLELNLEDVEPCVSGPKRPHDRVPLREMKVDWHACLNNKVGFKGFAVPKESQNKVAEFTFQGTPAHLRHGDVVIAAITSCTNTSNPSVMLGAALVAKKACELGLQVKPWIKTSLAPGSGVVTKYLQRSGLQKYLNELGFNIVGYGCTTCIGNSGDINEAVASAITENDIVAAAVLSGNRNFEGRVHPLTRANYLASPPLVVAYALAGTVDIDFDTEPIGIGKDGTKIFFRDIWPSSEEIANVVQSSVLPAMFRDTYNAITQGNPMWNNLSVPTGTLYAWDPTSTYIHEPPYFRDMSMSPPGSHGVKDAYCLLNFGDSITTDHISPAGSIHKDSPAARYLIERGVDRRDFNSYGSRRGNDEVMARGTFANIRIVNKFLNGEVGPKTIHIPSGEKLSVFDAAEKYKSEGHDMIILAGAEYGSGSSRDWAAKGPMLLGVKAVIAKSFERIHRSNLVGMGIIPLCFKPGDDADSLGLTGHERYTIDLPSNVNEIRPGQDVTVVTDAGKSFVSTLRFDTEVELAYFNHGGILQYVIRNMVNAKH

SEQ ID NO: 164 - protein, 9 aa, Glycine max
FLNGEVGPK

SEQ ID NO: 165 - protein, 9 aa, Glycine max
LSVFDAAEK

SEQ ID NO: 166 - protein, 9 aa, Glycine max
LSVFDVAEK

SEQ ID NO: 167 - protein, 12 aa, Glycine max
TSLAPGSGVVTK

SEQ ID NO: 168 - DNA, 2706 bp, Glycine max
  source 1..2706: mol_type = other DNA
atggcgactgagaaccctttcaatagcattttgaggacgctcgagaagcccggcggcgctggcgagtttggaaagtactttagcttgcccgctctcaacgatcctagaatcgatagacttccttactctgtgaggatacttttggaatctgcaatccgtaactgtgatgagtttcaagttaagagtaacgacattgaaaaaattattgattgggagaatacctctcccaagctggtggagattccattcaagccagctagagtgcttctgcaggactttactggggtgcctgctgtggttgatcttgcttgcatgcgagatgcaatgaacaaacttggtggtgattctaataaaattaaccccttggtaccagttgatcttgtcattgatcactctgttcaagttgatgtggcaagatcagaaaatgctgtccaggcaaatatggaacttgaattccaaagaaacaaggaaagatttggtttcctcaaatggggttctaatgcattcaataatatgcttgtcgttcctcctggatcagggatagttcatcaggttaatttagaataccttggtagagttgtgttcaacacaaatggtgtgctttatccagacagcgttgtcggaactgattcacacactactatgatagatggcctgggtgttgctggatggggagttggtggaatagaagcagaagctgcaatgcttggccagcccatgagcatggtcctaccgggtgttgttgggttcaaattattaggcaaactacgagatggtgtcacagctactgacttggtgttgactgttactcaaatgctaagaaagcatggggttgttggcaagtttgtagagttttatggggaaggcatgagtgaactgtctttggcagatcgtgccactatagcaaacatgtctcctgagtatggtgcaaccatgggcttctttcctgtggatcatgtcactttgcaatatttgagactgactggcagaagtgatgagactgtttctatgatagaatcctatttacgagcaaataagatgtttgtggattatagcgagccccaagtggagagagtgtactcatcctatttggaactcaatcttgaggatgtagagccctgtgtctcaggtccaaaaaggcctcacgaccgtgtccctttgagagaaatgaaggtagactggcatgcctgcctcaacaataaagttggatttaagggttttgctgtatcaaaagaatctcagaataaggttgcagagttcacattccaagggacaccagcacatcttaggcatggtgatgttgttatagctgctatcaccagttgtacaaatacttcaaatcctagcgtaatgcttggagctgcattggttgcaaagaaagcatgtgaattgggattgcaggtgaagccatggattaaaacaagtcttgccccaggttctggtgttgtaactaagtatttgcagaggagtggcttgcagaagtatttgaatgagctagggtttaatatagttgggtatggatgcaccacatgcattggaaattcaggggatattaatgaagctgttgcatctgcgattaccgaaaatgatatagtagctgcagctgtattgtctggaaataggaactttgagggccgagttcacccattaacaagagctaattatcttgcttctcctcctcttgttgttgcctatgcccttgctggcacagtggacattgactttgacactgaacccattgggatagggaaggatggaacagagatcttcttcaaggatatttggccatccagtgaagaaatagcaaatgttgtacaatcaagtgtgctgcctgatatgtttagggacacatataatgcaatcacccaagggaatcccatgtggaataatttatctgttccaacgggcactctttatgcttgggaccctacatcgacttatattcatgagccaccttattttagagatatgagcatgtctcccccaggctctcatggagtgaaggatgcttactgtttgctcaactttggagacagtatcacaactgaccacatctctccagctggcagcatacataaggacagtcctgcggccagataccttatagaacgtggggttgatagacgggacttcaactcctatggaagtcgccgtggtaatgatgaggtgatggcccgaggaacgtttgccaatattcgcattgtcaacaaatttttgaatggagaagttggacctaaaactattcatattccttctggggagaagttgtcagtctttgacgttgcagagaaatacaagagtgaagggcatgatatgattatcttggctggtgctgagtatgggagtggaagctctcgtgattgggctgcaaaggggccaatgctactgggcgtgaaagctgtcatagcaaaaagttttgaaaggattcaccgaagtaatttggtgggaatgggtatcattcctttgtgttttaagcccggggaggatgctgattctcttggattaactggtcaagaacgttacaccattgatctaccaagcaatgtgaatgaaatacgacccggtcaagatgtcacagtggtgacagacaccgggaaatcatttgtatctacattgagatttgatactgaggttgaactagcttacttcaaccatggaggtatcttacaatatgtcatcagaaacttgatcaatgctaaacattga

SEQ ID NO: 169 - protein, 901 aa, Glycine max
MATENPFNSILRTLEKPGGAGEFGKYFSLPALNDPRIDRLPYSVRILLESAIRNCDEFQVKSNDIEKIIDWENTSPKLVEIPFKPARVLLQDFTGVPAVVDLACMRDAMNKLGGDSNKINPLVPVDLVIDHSVQVDVARSENAVQANMELEFQRNKERFGFLKWGSNAFNNMLVVPPGSGIVHQVNLEYLGRVVFNTNGVLYPDSVVGTDSHTTMIDGLGVAGWGVGGIEAEAAMLGQPMSMVLPGVVGFKLLGKLRDGVTATDLVLTVTQMLRKHGVVGKFVEFYGEGMSELSLADRATIANMSPEYGATMGFFPVDHVTLQYLRLTGRSDETVSMIESYLRANKMFVDYSEPQVERVYSSYLELNLEDVEPCVSGPKRPHDRVPLREMKVDWHACLNNKVGFKGFAVSKESQNKVAEFTFQGTPAHLRHGDVVIAAITSCTNTSNPSVMLGAALVAKKACELGLQVKPWIKTSLAPGSGVVTKYLQRSGLQKYLNELGFNIVGYGCTTCIGNSGDINEAVASAITENDIVAAAVLSGNRNFEGRVHPLTRANYLASPPLVVAYALAGTVDIDFDTEPIGIGKDGTEIFFKDIWPSSEEIANVVQSSVLPDMFRDTYNAITQGNPMWNNLSVPTGTLYAWDPTSTYIHEPPYFRDMSMSPPGSHGVKDAYCLLNFGDSITTDHISPAGSIHKDSPAARYLIERGVDRRDFNSYGSRRGNDEVMARGTFANIRIVNKFLNGEVGPKTIHIPSGEKLSVFDVAEKYKSEGHDMIILAGAEYGSGSSRDWAAKGPMLLGVKAVIAKSFERIHRSNLVGMGIIPLCFKPGEDADSLGLTGQERYTIDLPSNVNEIRPGQDVTVVTDTGKSFVSTLRFDTEVELAYFNHGGILQYVIRNLINAKH

SEQ ID NO: 170 - protein, 9 aa, Glycine max
FLNGEVGPK

SEQ ID NO: 171 - protein, 9 aa, Glycine max
LSVFDVAEK

SEQ ID NO: 172 - protein, 12 aa, Glycine max
TSLAPGSGVVTK

SEQ ID NO: 173 - DNA, 2292 bp, Glycine max
  source 1..2292: mol_type = other DNA
atggcatctcacatcgttggatacccccgtatgggtcccaagagagagctcaagttcgctctcgagtctttctgggatggcaagagcagcgccgaggatttgcagaaggtgtcttctgatctcagggcatccatctggaagcagatggctgatgctgggatcaagtacatccccagcaacactttctctcactatgaccaggttctcgacgccaccgccaccctcggtgccgttccaccaaggtacggctggaccggcggcgagattgggtttgatacctacttctccatggccagaggtaatgctaccgtgccagctatggagatgaccaagtggttcgacaccaactaccactttattgtccctgaattgggccctgatgtgaacttcacctatgcttctcacaaggctgttgatgaatacaaggaggccaaggcgcttggagtggataccgttccggtcctcgttggccctgttacatacctgttgctctccaagcctgccaagggagttgagaaatccttttctctcctctctctccttcccaaggttcttgctgtctacaaggaagttattgctgaccttaaggcagctggtgcttcatggattcagtttgatgagcctacccttgtcttggaccttgagtctcacaagttgcaagcattcactgacgcatatgcagaacttgcgcctgctttgtctggtttgaatgttcttgttgagacctactttgctgacatccctgctgaggcatacaagaccctcacatctctgaatggcgtcactgcatatggatttgatttggtccgtggaaccaacactcttgatttgatcaagggtggatttcccagcggaaaatacctctttgctggagtggttgatggaaggaacatctgggccaatgaccttgctgcttctctcactaccttgcagggtcttgagggcattgtgggcaaagataagcttgttgtgtccacctcctcctcccttcttcacactgctgttgatctagttaacgagaccaagttggatgatgagatcaagtcatggctagcttttgctgcccaaaaaattgttgaagttaacgcattggctaaagcattgtctggccacaaggatgaggccttcttctctggtaatgctgctgctctggcttcaaggaagtcttctccaagagtgaccaacgaggctgttcagaaggctgctgctgcattgaagggttcagatcatcgccgtgcaacaaatgtcagtgccagactggattctcaacaaaagaagctcaaccttccaatcctgccaaccaccactattggatccttccctcagactgtagaactgaggagggtacgccgtgaattcaaggctaacaagatctccgaggaagagtatgtaaagtcaattaaggaggaaattcgcaaagttgttgagcttcaagaagagcttgatattgatgttcttgttcatggagaaccagagagaaatgatatggttgagtacttcggtgaacaattgtcaggctttgccttcaccgttaatgggtgggtgcaatcctatggttcccgttgcgtgaagccaccgatcatctatggtgatgtgagccgcccaaagccaatgaccgtcttctggtcatctctggctcagagctttaccaagcgcccaatgaagggaatgcttaccggtcctgttaccattctcaactggtcctttgttagaaatgaccaacctagatctgagaccacctaccagattgctttgtctatcaaggacgaagtggaagaccttgaaaaggctggcatcactgttatccaaattgatgaagctgctttgagagagggtcttccactgaggaaatcagagcaagctcactacttggactgggctgtccatgccttcagaatcaccaatgttggtgtccaggataccacccagatccacactcacatgtgctactcgaatttcaacgacatcatccactccatcatcgacatggacgccgatgttatcaccattgagaactctcgctccgacgagaagcttctgtcagtcttccgcgaaggtgtgaagtatggtgctggaattggccctggtgtctatgacatccactccccaagaataccaccaactgaagaaattgctgacagaatcaacaagatgctggcagtgctcgagaagaacatcttgtgggtgaaccctgactgtgggctcaagacccgtaagtacactgaggtgaagccagccctcacaaacatggttgccgcagcaaaactcatccgcaacgaacttgccaagtga

SEQ ID NO: 174 - protein, 763 aa, Glycine max
MASHIVGYPRMGPKRELKFALESFWDGKSSAEDLQKVSSDLRASIWKQMADAGIKYIPSNTFSHYDQVLDATATLGAVPPRYGWTGGEIGFDTYFSMARGNATVPAMEMTKWFDTNYHFIVPELGPDVNFTYASHKAVDEYKEAKALGVDTVPVLVGPVTYLLLSKPAKGVEKSFSLLSLLPKVLAVYKEVIADLKAAGASWIQFDEPTLVLDLESHKLQAFTDAYAELAPALSGLNVLVETYFADIPAEAYKTLTSLNGVTAYGFDLVRGTNTLDLIKGGFPSGKYLFAGVVDGRNIWANDLAASLTTLQGLEGIVGKDKLVVSTSSSLLHTAVDLVNETKLDDEIKSWLAFAAQKIVEVNALAKALSGHKDEAFFSGNAAALASRKSSPRVTNEAVQKAAAALKGSDHRRATNVSARLDSQQKKLNLPILPTTTIGSFPQTVELRRVRREFKANKISEEEYVKSIKEEIRKVVELQEELDIDVLVHGEPERNDMVEYFGEQLSGFAFTVNGWVQSYGSRCVKPPIIYGDVSRPKPMTVFWSSLAQSFTKRPMKGMLTGPVTILNWSFVRNDQPRSETTYQIALSIKDEVEDLEKAGITVIQIDEAALREGLPLRKSEQAHYLDWAVHAFRITNVGVQDTTQIHTHMCYSNFNDIIHSIIDMDADVITIENSRSDEKLLSVFREGVKYGAGIGPGVYDIHSPRIPPTEEIADRINKMLAVLEKNILWVNPDCGLKTRKYTEVKPALTNMVAAAKLIRNELAK

SEQ ID NO: 175 - protein, 21 aa, Glycine max
AGITVIQIDEAALREGLPLRK

SEQ ID NO: 176 - protein, 24 aa, Glycine max
ALGVDTVPVLVGPVTYLLLSKPAK

SEQ ID NO: 177 - protein, 16 aa, Glycine max
DEAFFSGNAAALASRK

SEQ ID NO: 178 - protein, 8 aa, Glycine max
DEVEDLEK

SEQ ID NO: 179 - protein, 8 aa, Glycine max
ISEEEYVK

SEQ ID NO: 180 - protein, 9 aa, Glycine max
IVEVNALAK

SEQ ID NO: 181 - protein, 8 aa, Glycine max
LIRNELAK

SEQ ID NO: 182 - protein, 10 aa, Glycine max
LLSVFREGVK

SEQ ID NO: 183 - protein, 21 aa, Glycine max
LVVSTSSSLLHTAVDLVNETK

SEQ ID NO: 184 - protein, 10 aa, Glycine max
SFSLLSLLPK

SEQ ID NO: 185 - protein, 12 aa, Glycine max
SSPRVTNEAVQK

SEQ ID NO: 186 - protein, 26 aa, Glycine max
TLTSLNGVTAYGFDLVRGTNTLDLIK

SEQ ID NO: 187 - protein, 29 aa, Glycine max
YGAGIGPGVYDIHSPRIPPTEEIADRINK

SEQ ID NO: 188 - DNA, 2196 bp, Glycine max
  source 1..2196: mol_type = other DNA
atggcttcttccacttcctctatctctctctcccaagccctcctccgccccaccaccacctctctcccctcctcctcctcctccctccgcctcacccccactctccgccctaccccacccgccctctccctccgccgccgcctcaccacctccatccgcgccgccgcctccgtcaaaaccgtcgagaaggcaaccgcggacgccgcgctggtcgaaaaatccgtcaacacgatccgcttcctcgccatcgacgccgtcgagaaggcgaattcgggtcacccggggctccccatgggatgcgctccgatgggtcacgtgctatatgatgaaaccatgaagtataaccctaagaaccctttctggttcaaccgcgatcgcttcgttctctccgctgggcacgggtgcatgcttcagtacgcgcttctgcacctcgctgggttcgatagcgtcaaggaagaggatttgagggaatttcgccaatgggggagcagaactccgggacaccctgagaattttgagactccgggaattgaagttacaacaggtcctcttggtcaggggattgccaatgctgttggtttggcccttgcagagaagcacttggctgcaagatacaacaagcctgacaatgagattgttgaccattacacgtatgctattctgggtgatggttgtcaaatggagggaattgcaaatgaagcatgctcacttgctggccactggggactggggaagctgatagcattttatgatgacaatcatatttctattgatggtaacacagagattgcgttcaccgagagtgttgatagtcgttttgagggacttgggtggcatgttatttgggtaaagaatggaaataatggctatgatgatattcgtgctgccattaaggaagcaaaagctgtcaaagacaaacccacattaatcaaggtcacaacaaccattggttatggttctcctaacaaggctaactcctacagtgtgcatggaagtgcactgggtgccaaagaagttgatgccaccaggcagaaccttggatggtcacatgagccattccacgtgcctgaggatgtcaaaaagcattggagtcgccacacccctgagggtgctgcacttgaagctgagtggaatgctaagtttgctgagtatgaaaagaaatacaaggaggaggctgcagaattgaaatctattatcaatggtgaattccctgctggttgggagaaagcacttccgacatacactccagagagcccagcggatgccaccagaaacctgtctcaaacaaaccttaatgcccttgcaaaggttcttcccggtctgcttggtggcagtgcagatcttgcttcttccaacatgaccttgctcaaaatgttcggggacttccaaaaggatactccagcagagcgtaatgttagattcggtgttagagaacacggaatgggagctatctgcaacggcattgctcttcacagccctggactgattccatattgtgcaaccttctttgtattcactgactacatgagaggtgccataaggctttctgcgctgtctgaggctggggttatttatgtcatgacccatgattcaataggacttggagaagatgggccaacccaccagccaattgagcacctagcaagcttccgggcaatgccaaacattttgatgcttcgtcccgccgacggtaacgaaacagccggagcatacaaagtggccgtgctcaacaggaagagaccctccattcttgccctatccaggcaaaaactgccccagcttcccggaacttccattgaaggagttgaaaagggtggttacaccatttcggacaactccactggcaacaagcctgatgtcattttgatcggaactggttcggaattggaaatcgctgccaaagctgctgatgacctaaggaaggaagggaaggctgttagagttgtttcccttgtttcttgggaactttttgatgagcaatcagaagcctacaaggagagtgttttccctgctgctgtttcagccagagttagcattgaggcaggatcaacatttgggtgggagaaaattgttggagcaaagggaaaagcaataggcattgatcgttttggagctagtgctccagctggaagaatatacaaagaatttggtatcactaaggaagctgttgttgctgcagctaaagagcttatctag

SEQ ID NO: 189 - protein, 731 aa, Glycine max
MASSTSSISLSQALLRPTTTSLPSSSSSLRLTPTLRPTPPALSLRRRLTTSIRAAASVKTVEKATADAALVEKSVNTIRFLAIDAVEKANSGHPGLPMGCAPMGHVLYDETMKYNPKNPFWFNRDRFVLSAGHGCMLQYALLHLAGFDSVKEEDLREFRQWGSRTPGHPENFETPGIEVTTGPLGQGIANAVGLALAEKHLAARYNKPDNEIVDHYTYAILGDGCQMEGIANEACSLAGHWGLGKLIAFYDDNHISIDGNTEIAFTESVDSRFEGLGWHVIWVKNGNNGYDDIRAAIKEAKAVKDKPTLIKVTTTIGYGSPNKANSYSVHGSALGAKEVDATRQNLGWSHEPFHVPEDVKKHWSRHTPEGAALEAEWNAKFAEYEKKYKEEAAELKSIINGEFPAGWEKALPTYTPESPADATRNLSQTNLNALAKVLPGLLGGSADLASSNMTLLKMFGDFQKDTPAERNVRFGVREHGMGAICNGIALHSPGLIPYCATFFVFTDYMRGAIRLSALSEAGVIYVMTHDSIGLGEDGPTHQPIEHLASFRAMPNILMLRPADGNETAGAYKVAVLNRKRPSILALSRQKLPQLPGTSIEGVEKGGYTISDNSTGNKPDVILIGTGSELEIAAKAADDLRKEGKAVRVVSLVSWELFDEQSEAYKESVFPAAVSARVSIEAGSTFGWEKIVGAKGKAIGIDRFGASAPAGRIYKEFGITKEAVVAAAKELI

SEQ ID NO: 190 - protein, 18 aa, Glycine max
AIGIDRFGASAPAGRIYK

SEQ ID NO: 191 - protein, 27 aa, Glycine max
ALPTYTPESPADATRNLSQTNLNALAK

SEQ ID NO: 192 - protein, 14 aa, Glycine max
ANSYSVHGSALGAK

SEQ ID NO: 193 - protein, 10 aa, Glycine max
ATADAALVEK

SEQ ID NO: 194 - protein, 7 aa, Glycine max
DKPTLIK

SEQ ID NO: 195 - protein, 30 aa, Glycine max
GGYTISDNSTGNKPDVILIGTGSELEIAAK

SEQ ID NO: 196 - protein, 14 aa, Glycine max
LPQLPGTSIEGVEK

SEQ ID NO: 197 - protein, 14 aa, Glycine max
NGNNGYDDIRAAIK

SEQ ID NO: 198 - protein, 15 aa, Glycine max
SVNTIRFLAIDAVEK

SEQ ID NO: 199 - protein, 12 aa, Glycine max
VTTTIGYGSPNK

SEQ ID NO: 200 - DNA, 1797 bp, Glycine max
  source 1..1797: mol_type = other DNA
atggaagttccttcggaggtaccatctactgacactggcaccagcaccggcaccggcgaaaccatgagcaagaatgcgttgaagcgggagctgaagaacaaacagagagaagaagaaaggaagcgcaaggatgaggagaaggccaaaaaggcagctgaagcacaacaggcaaaggataacaaatctgcaccagctgatgatgaggatatggatccaactcaataccttgagaacaggctaaagtatcttgcagttcaaaagacagaagggaagaacccgtatcctcataaattctttgtcactatgtctattgatcaatacatcaaggaatatggaggtttaagcaacgggcaacacattgaggatgtctctgtgtctttggctggccggatcatgcacaaacgcacctctggttctaagcttgtcttttatgacctgcacggtggtggttgcaaggtccaggttatggctgatgctagtaaatcagacttggatgaggctggattttccaaattccattctaatgtgaagcgcggtgacatagttggtgtcactgggttcccaggcaaaagtaagaagggcgaacttagtatttttcccaagaattttgtgtcgctgtctcattgtttgcacatgatgccaaggcaaaagtctgctgctgctgctattgataatagaaatccatggataccgggaagtactaggaatcctgaaacatatattttgaaggatcaggaaactagatatcggttacgccatttggatttgatgcttaatccagaggttcgagacatattcaagaccaggtctaaaatcatttcttacattaggaggttccttgatgaccttgatttcttggaggttgaaacacctatgatgaatatgattgctggtggagctgcagcccgtccatttgtaacacatcacaatgatcttaacatgaggttattcatgaggattgctccagaactgtatcttaaggagttggttgttggtggactggatcgtgtttatgaaattggtaaacaatttaggaatgagggcatagatttgactcataatcctgagtttactacctgtgagttctatatggcttataaggactacaatgacttaatggatctaacagagcaaatgttgagtggtatggttaaggagcttacgaaaggcagctataaaatcaagtatcatgcagatgggattgataaggaacctattgaaattgactttactacaccttttagaaggattgacatgattgaagagttagagaagatggcaggtctaagtattcccaaagacttgtcaagtgaggaagccaatcaatatttgaaggatgcatgcgtgaagtatgagatcaaatgtcctccccctgagacaactgctcgtttgttggataaacttgtgggtcactttttggaagaaacgtgtgtaaatcctacattcatcaaaaaccatcctgagatcatgagtcctctagcaaagtggcacagatcaaaaccaggcctgactgaacgttttgaattgtttgttaataaacgtgaactttgcaatgcatatactgaattgaatgaccctgtagtacaacgacagagatttgctgaacaactcaaggatcggcaatctggcgatgatgaagcaatggccttagatgaaacattttgtacggctctagaatatggtttaccacctactggtggttggggtttgggcattgatcggttgaccatgttactgactgattcacagaatattaaggaggttcttctcttccctgccatgaaacctcaagactga

SEQ ID NO: 201 - protein, 598 aa, Glycine max
MEVPSEVPSTDTGTSTGTGETMSKNALKRELKNKQREEERKRKDEEKAKKAAEAQQAKDNKSAPADDEDMDPTQYLENRLKYLAVQKTEGKNPYPHKFFVTMSIDQYIKEYGGLSNGQHIEDVSVSLAGRIMHKRTSGSKLVFYDLHGGGCKVQVMADASKSDLDEAGFSKFHSNVKRGDIVGVTGFPGKSKKGELSIFPKNFVSLSHCLHMMPRQKSAAAAIDNRNPWIPGSTRNPETYILKDQETRYRLRHLDLMLNPEVRDIFKTRSKIISYIRRFLDDLDFLEVETPMMNMIAGGAAARPFVTHHNDLNMRLFMRIAPELYLKELVVGGLDRVYEIGKQFRNEGIDLTHNPEFTTCEFYMAYKDYNDLMDLTEQMLSGMVKELTKGSYKIKYHADGIDKEPIEIDFTTPFRRIDMIEELEKMAGLSIPKDLSSEEANQYLKDACVKYEIKCPPPETTARLLDKLVGHFLEETCVNPTFIKNHPEIMSPLAKWHRSKPGLTERFELFVNKRELCNAYTELNDPVVQRQRFAEQLKDRQSGDDEAMALDETFCTALEYGLPPTGGWGLGIDRLTMLLTDSQNIKEVLLFPAMKPQD

SEQ ID NO: 202 - protein, 12 aa, Glycine max
DLSSEEANQYLK

SEQ ID NO: 203 - DNA, 1728 bp, Glycine max
  source 1..1728: mol_type = other DNA
atgtaccgctttgcaacctccctcgcctccaaagctagaattgccaggagcagcactcaacagattggaagtagggtcacttggaacaggaattatgcggccaaagacattaagtttggtgtggaggctcgggctctgatgcttaagggtgtcgaagagctcgctgatgccgttaaagtaacaatgggccctaaggggcgtaatgtggttattgagcaaagttttggagcacctaaagtgacgaaagatggagtaaccgttgctaagagcattgaattcaaggacaaagtcaagaatatcggtgccagtcttgtaaagcaggttgctaatgctactaatgacgtggctggtgatggaaccacttgtgctacaattcttactaaagcaatatttacggaaggatgtaaatcagttgcagctggaatgaatgcgatggacctgagacgaggtataaatatggctgttgatgctgtggtgacaaatctgaaaagcagagcacgtatgattagcacttctgaagaaattgcccaggttggaacaatatctgccaatggggagagggaaattggtgagttaattgcaaaagctatggagaaagttggcaaagagggtgtcatcacaatctcagatggcaagacattgtacaatgagttggaagtcgttgaaggaatgaagctcgacaggggatacatttctccatatttcataacaaaccagaagaaccagaaatgtgaacttgaggatcctctcattataatccatgaaaagaaaatctcaagcataaatgctatcgttaaagtattagagttagctttgaagagacaaagatctttattgattgtcgccgaggatgtggaaagtgatgcccttgcaactcttattctaaataaacttcgtgctggaatcaaggtatgtgccatcaaagctcctggttttggtgaaaaccgtaaatctggtctccaggatcttgctgttcttacaggaggtcaacttatcaccgaagagcttggtttgaatcttgaaaaagtagacctggacttatttggctcttgcaagaagataacaatttctaaagatgacactgtcatccttgatggagctggtgataagaaagcaattgaggaaagatgtgagcagattagatcagcaattgaaaatagcacttcagattatgacaaggaaaagttacaggaaagattagccaagctttctgggggtgttgcagtactgaagattggaggagccagtgaggctgaagttggtgagaagaaggatagagtgacagatgccttaaatgcaaccaaggcagctgtagaggagggcatagtacctggtggtggtgttgctcttctttatgcatcgagtgagttggataagcttcaaactgccaactttgatcaaaagataggtgtccagatcatccaaaatgctttgaagactcctgtgcacacaattgcttcaaatgcaggagttgagggtgctgttgttgttggcaaactattggaacagaatgatcctgatcttggatatgatgcagccaaaggtgaatatgttgatatggttaaaactggaattattgatccattgaaggttattaggaccgccttggttgatgcagccagtgtgtcatctttgatgacaacaactgaagctgttgtgtctgaactcccgaaggacgataaagatgttcctgccatgggtggtggcatgggtggcatggattattaa

SEQ ID NO: 204 - protein, 575 aa, Glycine max
MYRFATSLASKARIARSSTQQIGSRVTWNRNYAAKDIKFGVEARALMLKGVEELADAVKVTMGPKGRNVVIEQSFGAPKVTKDGVTVAKSIEFKDKVKNIGASLVKQVANATNDVAGDGTTCATILTKAIFTEGCKSVAAGMNAMDLRRGINMAVDAVVTNLKSRARMISTSEEIAQVGTISANGEREIGELIAKAMEKVGKEGVITISDGKTLYNELEVVEGMKLDRGYISPYFITNQKNQKCELEDPLIIIHEKKISSINAIVKVLELALKRQRSLLIVAEDVESDALATLILNKLRAGIKVCAIKAPGFGENRKSGLQDLAVLTGGQLITEELGLNLEKVDLDLFGSCKKITISKDDTVILDGAGDKKAIEERCEQIRSAIENSTSDYDKEKLQERLAKLSGGVAVLKIGGASEAEVGEKKDRVTDALNATKAAVEEGIVPGGGVALLYASSELDKLQTANFDQKIGVQIIQNALKTPVHTIASNAGVEGAVVVGKLLEQNDPDLGYDAAKGEYVDMVKTGIIDPLKVIRTALVDAASVSSLMTTTEAVVSELPKDDKDVPAMGGGMGGMDY

SEQ ID NO: 205 - protein, 24 aa, Glycine max
AAVEEGIVPGGGVALLYASSELDK

SEQ ID NO: 206 - protein, 11 aa, Glycine max
DRVTDALNATK

SEQ ID NO: 207 - protein, 10 aa, Glycine max
EGVITISDGK

SEQ ID NO: 208 - protein, 14 aa, Glycine max
GRNVVIEQSFGAPK

SEQ ID NO: 209 - protein, 10 aa, Glycine max
GVEELADAVK

SEQ ID NO: 210 - protein, 12 aa, Glycine max
IGGASEAEVGEK

SEQ ID NO: 211 - protein, 11 aa, Glycine max
IGVQIIQNALK

SEQ ID NO: 212 - protein, 9 aa, Glycine max
ISSINAIVK

SEQ ID NO: 213 - protein, 15 aa, Glycine max
LLEQNDPDLGYDAAK

SEQ ID NO: 214 - protein, 9 aa, Glycine max
LQTANFDQK

SEQ ID NO: 215 - protein, 9 aa, Glycine max
LSGGVAVLK

SEQ ID NO: 216 - protein, 8 aa, Glycine max
NIGASLVK

SEQ ID NO: 217 - protein, 8 aa, Glycine max
TGIIDPLK

SEQ ID NO: 218 - protein, 20 aa, Glycine max
TPVHTIASNAGVEGAVVVGK

SEQ ID NO: 219 - DNA, 1725 bp, Glycine max
  source 1..1725: mol_type = other DNA
atgtatcgctttgcttctaacctcgcttccaaagcaaggattgctaggagtagcagccaccagattggaagtaggctgagttcgagcagaaactatgcggctaaagacattagatttggtgtggaggctcgggcactgatgcttaagggtgttgaagagctagctgatgctgtcaaagtaaccatgggtcccaaggggcgtaatgttgtgattgagcaaagttttggtgcccctaaagtaaccaaagatggagtaactgttgcaaagagcattgaattcaaggataaagttaagaatgttggtgccagtcttgtaaagcaggttgcaaatgctactaatgatgtggctggtgatggaaccacgtgcgctacagtccttacacgagcaatatttactgaaggctgcaaatcagttgcggctggaatgaatgcaatggacctgaggcgtggtataagtatggctgtcgatgctgtggtgacaaacttgaaaagcagagcaaggatgattagcacatctgaagaaatagcacaggtagggacaatatctgctaacggagagagagaaattggtgagttgattgcaaaagctatggagaaagttggcaaagagggagtaatcacaatttcagatggcaaaaccttatataatgagttggaagttgttgaaggaatgaagctcgacaggggctacatatctccttatttcattacaaatgataagaaccagaaatgtgaacttgaagatcccctcattctaattcatgagaagaaaatctcgagtataaatgctatagttaaagtcttagagttggctttgaagagacaaagatctttattgattattgccgaggatgtggaaagtgatgcacttgcaactcttattctaaataagcttcgggctggaattaaggtatgtgctattaaagcccctggttttggcgaaaataggaaagccaatcttcaagatcttgcagttctcacaggaggagcacttattactgaagagcttggcttgaagcttgaaaaagtggatttggatatgcttggcacctgtaaaaaaataacagtttctaaggatgacactgtcattcttgacggagcaggtgacaagaaagcacttgaggaaagatgcgagcagattaggtctgcaattgaaaacagcacctcagattatgacaaggaaaagttacaggaaagattagccaagctttctggtggtgttgccgtgcttaagattggaggagccagtgaagctgaagttggtgagaagaaggatagagtgacagatgccttaaatgcaactaaggctgctgtagaggaaggcattgtatcaggtggtggtgttgctcttctatatgcgtcaaaagagttggataaacttcaaactgctaactttgatcagaagattggtgtccagattatccaaaatgctttaaagacacctgtgctcacaattgcatcaaatgcgggtgttgagggtgcagttgttgttggcaaactattggaacaggagaatcatgatctaggatatgatgcagccaaaggtgaatatgttgatatggttaaagctggaatcatagaccctttgaaggtgattaggacagccttggttgatgcagccagtgtatcatctttgatgacaacaactgaagctgttgtgtctgaacttccaaacgatgataaagatactccagctatggctggtggcatgggtggcatgggttat

SEQ ID NO: 220 - protein, 575 aa, Glycine max
MYRFASNLASKARIARSSSHQIGSRLSSSRNYAAKDIRFGVEARALMLKGVEELADAVKVTMGPKGRNVVIEQSFGAPKVTKDGVTVAKSIEFKDKVKNVGASLVKQVANATNDVAGDGTTCATVLTRAIFTEGCKSVAAGMNAMDLRRGISMAVDAVVTNLKSRARMISTSEEIAQVGTISANGEREIGELIAKAMEKVGKEGVITISDGKTLYNELEVVEGMKLDRGYISPYFITNDKNQKCELEDPLILIHEKKISSINAIVKVLELALKRQRSLLIIAEDVESDALATLILNKLRAGIKVCAIKAPGFGENRKANLQDLAVLTGGALITEELGLKLEKVDLDMLGTCKKITVSKDDTVILDGAGDKKALEERCEQIRSAIENSTSDYDKEKLQERLAKLSGGVAVLKIGGASEAEVGEKKDRVTDALNATKAAVEEGIVSGGGVALLYASKELDKLQTANFDQKIGVQIIQNALKTPVLTIASNAGVEGAVVVGKLLEQENHDLGYDAAKGEYVDMVKAGIIDPLKVIRTALVDAASVSSLMTTTEAVVSELPNDDKDTPAMAGGMGGMGY

SEQ ID NO: 221 - protein, 8 aa, Glycine max
AGIIDPLK

SEQ ID NO: 222 - protein, 11 aa, Glycine max
DRVTDALNATK

SEQ ID NO: 223 - protein, 10 aa, Glycine max
EGVITISDGK

SEQ ID NO: 224 - protein, 14 aa, Glycine max
GRNVVIEQSFGAPK

SEQ ID NO: 225 - protein, 10 aa, Glycine max
GVEELADAVK

SEQ ID NO: 226 - protein, 12 aa, Glycine max
IGGASEAEVGEK

SEQ ID NO: 227 - protein, 11 aa, Glycine max
IGVQIIQNALK

SEQ ID NO: 228 - protein, 9 aa, Glycine max
ISSINAIVK

SEQ ID NO: 229 - protein, 15 aa, Glycine max
LLEQENHDLGYDAAK

SEQ ID NO: 230 - protein, 9 aa, Glycine max
LQTANFDQK

SEQ ID NO: 231 - protein, 9 aa, Glycine max
LSGGVAVLK

SEQ ID NO: 232 - DNA, 1869 bp, Glycine max
  source 1..1869: mol_type = other DNA
atggctcaagttcttgagaaccccaccattgacgcggcggcttccggcgcgaacccctcgctgacgaccatctcgttgtacgtcggggatcttcaccacgacgtcaacgatccgcagctgtatgatctcttcaaccaggttgcccaagtcgtttccgttcgaatttgcagggacgtggccacccaacaatcccttggttacggctacgtcaatttcagcaacgctcacgatgccgcaaaagcaattgatgtgctgaatttcactccactgaacggcaaaatcattaggataatgtactctattcgggaccctagtgctcggaagagtggtgcagctaatgttttcataaagaatttggacaaggcgattgatcacaaggctttgtacgatacattttcggcttttgggaatattctatcttgcaaggttgcgacagatgcttctggccagtctaaaggccatggttttgtgcagtttgagagtgaagaatctgcacaaaatgcaattgacaagttaaacggcatgctgatcaatgataagcaggtctttgtgggtcctttcctgcggaagcaggatagagagagtgctctcagtggcacaaagtttaataacgttttcgtgaaaaacctgttggattcaatgactgaggctgacttggagagaatttttggagaatatggagctattactagcgctgtagtgatgagagatgtagatggtaaatcaaaaggttttggttttgtcaattttgcaaatgtagacgatgctgctaaagctgtggaggctcttaatggaaagaattttgatggtaaggagtggtatgttggaaaagcccagaaaaagtctgaaagggagcttgaactgaaagggcaacatgaacagattacaaaggagactgttgataaatatcatggtacaaacttgtatatcaagaacttggatgatagtgttggtgatgaagaactcatggaactgttctctgagtttggtacgataacctcatgcaaggttatgcgagatccaaatggaattagtagaggatctggatttgtttcattttcaattgctgagggagcaactcgggctcttggtgagatgaatggtaaaatggttgctggcaaacctctctatgttgcccttgcacagagaaaagaagatagaagagcaaggttacaggcacaattttcacaatcgaggcctgctgcaataacccctaatgtttcacctcgaatgcctctataccctctaggggctcctgcgataggacaacaatttttgtatgggcaagcagctccagccacgatacctcaacattttgttcctggaatgaggccaggtggtgctccaaacttctatgttccactggttcagcagggtcaacagggacagcgcccaggaggacaccgaggatcaggtcctatgcaacagctccagcagccattgccattgatgcaccaacagatgcttccaaggggacatgtctatcgatatccttctggctccaatatgcaaaatattcaactggcaggagtacctggaggaatggtcacttatggcattggtcaacaaatgcccacgcaggccctggcctcggcccttgccaatgctactcctgaacagcagaggactatgcttggtgaagctttatacccgttagtagataagctggagcatgaagcagcagcaaaggttactggtatgctcttagagatggatcagcctgaggtattacatctgattgagtcaccggatgctctcaaggctaaagttgttgaggccatggatgtgttgaagaaagttacacagcagcaatcaaacagcccagccgatcaactggcttcactctctctgaatgataatgtcgagtct

SEQ ID NO: 233 - protein, 623 aa, Glycine max
MAQVLENPTIDAAASGANPSLTTISLYVGDLHHDVNDPQLYDLFNQVAQVVSVRICRDVATQQSLGYGYVNFSNAHDAAKAIDVLNFTPLNGKIIRIMYSIRDPSARKSGAANVFIKNLDKAIDHKALYDTFSAFGNILSCKVATDASGQSKGHGFVQFESEESAQNAIDKLNGMLINDKQVFVGPFLRKQDRESALSGTKFNNVFVKNLLDSMTEADLERIFGEYGAITSAVVMRDVDGKSKGFGFVNFANVDDAAKAVEALNGKNFDGKEWYVGKAQKKSERELELKGQHEQITKETVDKYHGTNLYIKNLDDSVGDEELMELFSEFGTITSCKVMRDPNGISRGSGFVSFSIAEGATRALGEMNGKMVAGKPLYVALAQRKEDRRARLQAQFSQSRPAAITPNVSPRMPLYPLGAPAIGQQFLYGQAAPATIPQHFVPGMRPGGAPNFYVPLVQQGQQGQRPGGHRGSGPMQQLQQPLPLMHQQMLPRGHVYRYPSGSNMQNIQLAGVPGGMVTYGIGQQMPTQALASALANATPEQQRTMLGEALYPLVDKLEHEAAAKVTGMLLEMDQPEVLHLIESPDALKAKVVEAMDVLKKVTQQQSNSPADQLASLSLNDNVES

SEQ ID NO: 234 - protein, 13 aa, Glycine max
AIDVLNFTPLNGK

SEQ ID NO: 235 - protein, 7 aa, Glycine max
FNNVFVK

SEQ ID NO: 236 - protein, 15 aa, Glycine max
GFGFVNFANVDDAAK

SEQ ID NO: 237 - protein, 9 aa, Glycine max
SGAANVFIK

SEQ ID NO: 238 - DNA, 1851 bp, Glycine max
  source 1..1851: mol_type = other DNA
atggattctgattatggtattcccagggaactctcagatcttcaaaagattcggtctttgtaccagccagagctccctccttgtctccagggaaccactgtgagggttgaatttggtgacgcaaccaccactgctgaccccactgatgcactcaccgtctgcagggcttttcctaacacttatggacaccctttggctcactttctcagggccactgccaaagtccctgatgctcagatcataactgagttccctcctattagggtggggattgtcttttgtgggagacaatctcctggagggcataatgtcatttggggtatccacaatgctctcaaaattcacaatcccaacagtgttttgcttggatttcttggtggttcagaaggtctatttgcccagaaaactctggagataactgacgatattctttcgacgtacaaaaatcaaggtggttatgatttgcttgggcggacgaaggatcaaattaggaccacagaacaagttaatgctgcacttgctgcatgcaacaatttgaaacttgatggccttgtcataatcggaggggtgacatcaaacacagatgctgcacagcttgctgaaacatttgttgtagcaaaatgccccacaaaggtggttggtgttcctgtcactctgaatggagatcttaaaaaccagtttgtagaaactaatgttggttttgatacaatttgtaaggttaattctcagctcatcagcaatgtctgcactgatgctctctctgcagagaagtattattatttcatccgtcttatgggacgcaaggcatcacacgttgctttggaatgcaccctccagtcccatccaaatatggtaattcttggtgaggaggttgctgcatcaaagcttaccctttctgaaattgcaaaacagattactgatgcaattcaggctagagcagagcaagacaaataccatggagtcattctccttccagaagggctgatagagagtattcctgaagtgtatgccctcttgaaggaaattcatggtttactcagacaaggtgttgaagttgacaagatatcttctcagctaacaccatgggcatctgctttatttgaatttctgccaccctttattaggagacagttgctcctttaccctgaatccgacgactcagcacagttatcacagattgaaactgagaaattactagcatatcttgtggaagtcgagattaacaagcgtctgaaagaaggcacatacaaggggaagaaattcaatgccatttgccacttttttggttatcaagctcgtggatctctgccatcaaaatttgactgtgattacgcatatgtccttgggcacatctgctaccatatactggccgctggtctaaatggatacatggcaactttaactaacctgaagaatccagtaaacaagtggaaatgtggggctgctccaattgcatctatgatgactgtaaagcgctggtctccaaatccaggagctacatcaattggaaaacctgctattcatccagctactgtggacttgagaggcaaagcatatgagctgttgagacaaaaggcacaaagctttcttatggatgacatctacagaaatccaggtccacttcagtttgatggtcccggcgcagatgccaaggttataactctttctgttgaagaccaggactacatgggccgcattaagaaacttcaggagtaccttgaacaggttcgaaccattgtcaagccaggatgccctcaggaggttttgaaagctgctctaagcgtcatggcgtcggttaccgaagttctagctgcaatgtcaacatcttataccaacaccctggcatccctg

SEQ ID NO: 239 - protein, 617 aa, Glycine max
MDSDYGIPRELSDLQKIRSLYQPELPPCLQGTTVRVEFGDATTTADPTDALTVCRAFPNTYGHPLAHFLRATAKVPDAQIITEFPPIRVGIVFCGRQSPGGHNVIWGIHNALKIHNPNSVLLGFLGGSEGLFAQKTLEITDDILSTYKNQGGYDLLGRTKDQIRTTEQVNAALAACNNLKLDGLVIIGGVTSNTDAAQLAETFVVAKCPTKVVGVPVTLNGDLKNQFVETNVGFDTICKVNSQLISNVCTDALSAEKYYYFIRLMGRKASHVALECTLQSHPNMVILGEEVAASKLTLSEIAKQITDAIQARAEQDKYHGVILLPEGLIESIPEVYALLKEIHGLLRQGVEVDKISSQLTPWASALFEFLPPFIRRQLLLYPESDDSAQLSQIETEKLLAYLVEVEINKRLKEGTYKGKKFNAICHFFGYQARGSLPSKFDCDYAYVLGHICYHILAAGLNGYMATLTNLKNPVNKWKCGAAPIASMMTVKRWSPNPGATSIGKPAIHPATVDLRGKAYELLRQKAQSFLMDDIYRNPGPLQFDGPGADAKVITLSVEDQDYMGRIKKLQEYLEQVRTIVKPGCPQEVLKAALSVMASVTEVLAAMSTSYTNTLASL

SEQ ID NO: 240 - protein, 13 aa, Glycine max
VVGVPVTLNGDLK

SEQ ID NO: 241 - DNA, 1575 bp, Glycine max
  source 1..1575: mol_type = other DNA
atgcccaaattttttcatagcattttcagaggaacaatggcgggtagggtttcgacgtgcttcttcttcgtgtttgctctttctctccttcttccttttcagatctccgctgaggaatcatcggagaaagagttcgtactcacattggatcactctaactttcacgacactgtcagcaagcacgatttcatcgtcgtcgagttctacgctccatggtgtggccactgtaagaagcttgctcccgagtatgagaaagctgcttctatcttgagtagtcatgatcctccggttgttttggctaaaattgatgccaatgaggagaagaacaaagaccttgcatctcaatatgatgttaggggatacccaacaattaagattctgagaaatgggggaaagaatgttcaggaatacaaaggtccccgtgaagcagatggcatcgttgactatttgaagaaacaaagtggtcctgcttcaactgaaattaaatctgctgatgaagctactgctttcattggtgaaaataaagttgctattgttggagttttcccgaaattttctggagaggagtttgacaacttttctgccttagcagagaagttgcgttctgactatgacttcggtcacactctgaatgccaaacaccttccaagaggagagtcatcagtttctggtcccgtagttaggttgttcaagccattcgatgaactttttgttgacttccaggatttcaatgtggaagctctagagaaatttgttgaggaatctagtacccctgttgttactgtctttaacaatgatccgagcaatcaccctttcgttgccaaattcttcaacagtcccaatgcaaaggcaatgttgttcatcaacttcactgccgaaggtgctgaatctttcaaatcaaaataccgtgaagctgctgagcaacataaacaacagggtgtaagctttttggtgggagatgttgagtctagtcaaggtgctttccagtattttggactgaaagaagaacaagtacctctaattatcattcaacacaatgacgggaaaaagttttttaaacccaatttggaagctgatcacattccaacttggttgaaggcgtacaaggatggtaatgttgcaccatttgtgaagtctgaacccattcctgaagctaacgatgaacctgttaaagtggtagttgggaacagtcttgaggacattgttttcaaatctgggaagaatgttctgctggagttttatgctccctggtgtggtcattgcaaacagttggctccaatattggacgaagttgctatctcatatcaaagtgatgctgatgttgttattgcaaaactggatgcaactgccaacgatatcccaagtgaaacctttgatgtccaaggttatccaaccgtgtacttcaggtcagcaagtggaaagttatcacaatacgaagggggtaggaccaaggaagacatcatagaattcatcgaaaagaaccgggacaaacctgctcagcaagaacaaggacaagataaacctgctcagcaagaacaaggacaagatgagcaagaaaaaggaaaagatgagctt

SEQ ID NO: 242 - protein, 525 aa, Glycine max
MPKFFHSIFRGTMAGRVSTCFFFVFALSLLLPFQISAEESSEKEFVLTLDHSNFHDTVSKHDFIVVEFYAPWCGHCKKLAPEYEKAASILSSHDPPVVLAKIDANEEKNKDLASQYDVRGYPTIKILRNGGKNVQEYKGPREADGIVDYLKKQSGPASTEIKSADEATAFIGENKVAIVGVFPKFSGEEFDNFSALAEKLRSDYDFGHTLNAKHLPRGESSVSGPVVRLFKPFDELFVDFQDFNVEALEKFVEESSTPVVTVFNNDPSNHPFVAKFFNSPNAKAMLFINFTAEGAESFKSKYREAAEQHKQQGVSFLVGDVESSQGAFQYFGLKEEQVPLIIIQHNDGKKFFKPNLEADHIPTWLKAYKDGNVAPFVKSEPIPEANDEPVKVVVGNSLEDIVFKSGKNVLLEFYAPWCGHCKQLAPILDEVAISYQSDADVVIAKLDATANDIPSETFDVQGYPTVYFRSASGKLSQYEGGRTKEDIIEFIEKNRDKPAQQEQGQDKPAQQEQGQDEQEKGKDEL

SEQ ID NO: 243 - protein, 16 aa, Glycine max
AASILSSHDPPVVLAK

SEQ ID NO: 244 - protein, 9 aa, Glycine max
DGNVAPFVK

SEQ ID NO: 245 - protein, 15 aa, Glycine max
DLASQYDVRGYPTIK

SEQ ID NO: 246 - protein, 9 aa, Glycine max
EDIIEFIEK

SEQ ID NO: 247 - protein, 15 aa, Glycine max
EEQVPLIIIQHNDGK

SEQ ID NO: 248 - protein, 17 aa, Glycine max
EFVLTLDHSNFHDTVSK

SEQ ID NO: 249 - protein, 16 aa, Glycine max
FFKPNLEADHIPTWLK

SEQ ID NO: 250 - protein, 8 aa, Glycine max
FFNSPNAK

SEQ ID NO: 251 - protein, 15 aa, Glycine max
FSGEEFDNFSALAEK

SEQ ID NO: 252 - protein, 25 aa, Glycine max
FVEESSTPVVTVFNNDPSNHPFVAK

SEQ ID NO: 253 - DNA, 1509 bp, Glycine max
  source 1..1509: mol_type = other DNA
atggcgggtacggtttcgacttgtttcttcttcgtgtttcttctttctctccttcttccttttcagatctccgctgaggaatcctcggagaaggagttcgtactcacattggatcactctaactttcacgacactgtcagcaagcacgatttcatcgtcgtcgagttctacgccccatggtgtggtcactgtaagaagcttgctccggagtatgagaaagctgcttctatcttgagtagtcatgatcctcctattgttttggctaaagtagatgccaatgaggagaagaacaaagaccttgcatcacaatatgatgttaagggattcccaacaattaatattctgagaaatgggggaaagaatgttcaggaatacaaaggtccccgtgaagcagatggcatcgttgactatttgaagaaacaaagtggtcctgcttcaactgaaattaaatctgctgatgaagctactgcttttattggtgaaaataaagttgctattgttggagttttcccgaaattttctggagaggagtttgacaacttttctgccttagcagagaagttgcgttctgattatgactttggtcacactctgaatgccaaactccttccaagaggagagtcatcagtttctgggcccgtagttaggctgttcaagccattcgatgaactttttgttgacttccaggatttcaatgtggaagctctagagaagtttgttgaggaatctagtacccctgttgttactgtctttaacaatgaaccgagcaatcaccctttcgttgtcaaattcttcaacagtcccaacgcaaaggcaatgttgttcatcaacttcactgccgaaggtgctgaagctatcaaatcaaaataccgtgaagctgctgagcaatataaacaacagggtgtcagctttttggtgggagatgttgagtctagtcaaggtgctttccagtattttggactgaaagaagagcaagtacctctaattatcattcaacataatgatgggaaaaagttttttaaacccaatttggaagctgatcacattccaacctggttgaaggcatacaaggatggtcatgttgcaccatttgttaagtctgaacccattcccgagactaacgatgaacctgttaaagtggtagttggggccagtcttgaggacattgttttcaaatctgggaagaatgttctgctggagttttatgctccctggtgtggtcattgcaaacagttggctccaatattggatgaagttgctatctcatatcaaaatgaagctgatgttgttattgcaaaactggatgcaactgccaacgatatcccaagtgaaacctttgatgtccaaggttatcctaccgtgtacttcaggtcagcaagtggaaagttatcacaatacgacggcggtaggaccaaggaagacatcatagaattcatcgaaaagaaccgggataaacctgctcagcaagaacaaggaaaagatgagcaagaacaaggaaaagatgagctt

SEQ ID NO: 254 - protein, 503 aa, Glycine max
MAGTVSTCFFFVFLLSLLLPFQISAEESSEKEFVLTLDHSNFHDTVSKHDFIVVEFYAPWCGHCKKLAPEYEKAASILSSHDPPIVLAKVDANEEKNKDLASQYDVKGFPTINILRNGGKNVQEYKGPREADGIVDYLKKQSGPASTEIKSADEATAFIGENKVAIVGVFPKFSGEEFDNFSALAEKLRSDYDFGHTLNAKLLPRGESSVSGPVVRLFKPFDELFVDFQDFNVEALEKFVEESSTPVVTVFNNEPSNHPFVVKFFNSPNAKAMLFINFTAEGAEAIKSKYREAAEQYKQQGVSFLVGDVESSQGAFQYFGLKEEQVPLIIIQHNDGKKFFKPNLEADHIPTWLKAYKDGHVAPFVKSEPIPETNDEPVKVVVGASLEDIVFKSGKNVLLEFYAPWCGHCKQLAPILDEVAISYQNEADVVIAKLDATANDIPSETFDVQGYPTVYFRSASGKLSQYDGGRTKEDIIEFIEKNRDKPAQQEQGKDEQEQGKDEL

SEQ ID NO: 255 - protein, 16 aa, Glycine max
AASILSSHDPPIVLAK

SEQ ID NO: 256 - protein, 9 aa, Glycine max
DGHVAPFVK

SEQ ID NO: 257 - protein, 9 aa, Glycine max
DLASQYDVK

SEQ ID NO: 258 - protein, 9 aa, Glycine max
EDIIEFIEK

SEQ ID NO: 259 - protein, 15 aa, Glycine max
EEQVPLIIIQHNDGK

SEQ ID NO: 260 - protein, 17 aa, Glycine max
EFVLTLDHSNFHDTVSK

SEQ ID NO: 261 - protein, 16 aa, Glycine max
FFKPNLEADHIPTWLK

SEQ ID NO: 262 - protein, 8 aa, Glycine max
FFNSPNAK

SEQ ID NO: 263 - protein, 15 aa, Glycine max
FSGEEFDNFSALAEK

SEQ ID NO: 264 - protein, 25 aa, Glycine max
FVEESSTPVVTVFNNEPSNHPFVVK

SEQ ID NO: 265 - protein, 13 aa, Glycine max
GFPTINILRNGGK

SEQ ID NO: 266 - protein, 13 aa, Glycine max
GPREADGIVDYLK

SEQ ID NO: 267 - protein, 7 aa, Glycine max
LAPEYEK

SEQ ID NO: 268 - protein, 29 aa, Glycine max
LDATANDIPSETFDVQGYPTVYFRSASGK

SEQ ID NO: 269 - protein, 14 aa, Glycine max
LRSDYDFGHTLNAK

SEQ ID NO: 270 - protein, 10 aa, Glycine max
LSQYDGGRTK

SEQ ID NO: 271 - protein, 23 aa, Glycine max
QLAPILDEVAISYQNEADVVIAK

SEQ ID NO: 272 - protein, 24 aa, Glycine max
QQGVSFLVGDVESSQGAFQYFGLK

SEQ ID NO: 273 - protein, 10 aa, Glycine max
QSGPASTEIK

SEQ ID NO: 274 - protein, 13 aa, Glycine max
SADEATAFIGENK

SEQ ID NO: 275 - protein, 13 aa, Glycine max
SEPIPETNDEPVK

SEQ ID NO: 276 - protein, 9 aa, Glycine max
VAIVGVFPK

SEQ ID NO: 277 - protein, 13 aa, Glycine max
VVVGASLEDIVFK

SEQ ID NO: 278 - protein, 9 aa, Glycine max
YREAAEQYK

SEQ ID NO: 279 - DNA, 1467 bp, Glycine max
  source 1..1467: mol_type = other DNA
atgggtgtggctcaaaacattcccgatgcggaggagggaaccttggagattggaatggaatacagaactgtttctggagttgctgggccattggtcattcttgataaagttaagggacccaagtttcaagagattgttaacattcgcttaggagatggtactactcggcgtggacaagtgctagaagttgatggtgaaaaggctgttgttcaggtctttgagggtacatctgggattgacaataaatttacaactgtgcaatttactggagaggtgttaaaaactccagtatcattggacatgcttggccgcatattcaatggttctggaaaaccaattgataatggtccacccattttacctgaggcttacttggacatatcaggaagttccatcaaccctagtgagagaacctatccagaggagatgatacagacaggaatatctaccattgatgtcatgaattctattgctagaggtcaaaagatccctctattctcagctgctggtctcccccataatgaaattgctgcacagatatgccgtcaggctggcttagtcaagcgactagaaaagtctgataatcttctcgagggtggaggagaagaggacaattttgccattgtgtttgcagctatgggagttaacatggagactgcacagtttttcaaacgtgactttgaggaaaatggctcgatggagagagtgacccttttccttaatctggcaaatgatcctacgattgagcgtatcatcactcctcgtattgctcttactactgctgaatatttggcatatgaatgtggcaagcatgttcttgtgatactaactgatatgagttcttatgctgatgctcttcgtgaggtatctgctgcccgagaagaagtacctggtagacgtgggtatcccggctacatgtatacagatcttgcaacaatatacgaacgtgctggaagaattgaggggcgaaaaggctctattacacaaattccaattctaaccatgcctaatgatgatattacgcatccaactcctgatcttacaggatatattactgagggacagatatatatcgacagacagttgtataacaggcagatatatcctccaatcaatgtccttccatcattgtctcgtttgatgaagagtgccattggtgagggaatgacacgaaaggatcattctgatgtgtccaaccagctctatgcaaattatgccattggaaaggatgtccaggcaatgaaagccgtggtcggagaagaagcactttcatctgaggacctgctgtatttggaatttttggagaagtttgagaggaagtttgttgcccaaggagcatacgatacacgcaatatcttccagtcactagatcttgcatggactttgcttcggattttccctcgcgagcttctccaccgtattcctgcaaagactcttgatcagttctacagccgagatgccagtaac

SEQ ID NO: 280 - protein, 489 aa, Glycine max
MGVAQNIPDAEEGTLEIGMEYRTVSGVAGPLVILDKVKGPKFQEIVNIRLGDGTTRRGQVLEVDGEKAVVQVFEGTSGIDNKFTTVQFTGEVLKTPVSLDMLGRIFNGSGKPIDNGPPILPEAYLDISGSSINPSERTYPEEMIQTGISTIDVMNSIARGQKIPLFSAAGLPHNEIAAQICRQAGLVKRLEKSDNLLEGGGEEDNFAIVFAAMGVNMETAQFFKRDFEENGSMERVTLFLNLANDPTIERIITPRIALTTAEYLAYECGKHVLVILTDMSSYADALREVSAAREEVPGRRGYPGYMYTDLATIYERAGRIEGRKGSITQIPILTMPNDDITHPTPDLTGYITEGQIYIDRQLYNRQIYPPINVLPSLSRLMKSAIGEGMTRKDHSDVSNQLYANYAIGKDVQAMKAVVGEEALSSEDLLYLEFLEKFERKFVAQGAYDTRNIFQSLDLAWTLLRIFPRELLHRIPAKTLDQFYSRDASN

SEQ ID NO: 281 - protein, 17 aa, Glycine max
DHSDVSNQLYANYAIGK

SEQ ID NO: 282 - protein, 26 aa, Glycine max
FQEIVNIRLGDGTTRRGQVLEVDGEK

SEQ ID NO: 283 - protein, 12 aa, Glycine max
FTTVQFTGEVLK

SEQ ID NO: 284 - protein, 12 aa, Glycine max
TLDQFYSRDASN

SEQ ID NO: 285 - DNA, 1464 bp, Glycine max
  source 1..1464: mol_type = other DNA
atgggtgtggctgagaacattcccgatatggaggagggtaccttggagattggaatggaatacagaactgtatctggagttgctggaccattggtcattcttgacaaagttaagggacccaaatttcaagagattgttaatattcgtttaggagatggaactactcggcgtggacaagttctagaggttgatggcgaaaaggctgttgttcaggtatttgaaggcacatctgggatcgacaataagtttacaactgtgcaatttactggagaggtgttaaaaactcctgtctcactggacatgcttggacgcatatttaatggttctggaaaaccaattgataatggtccaccaattttacctgaggcttacctggatatttctggaagttccatcaatcctagtgagagaacctatcctgaggagatgatacagacaggaatatctacaattgatgtcatgaattctattgctagaggtcaaaagatccctctattctcagcagctggtctcccccataatgaaattgctgcacagatctgtcgtcaggctggcttagtcaagcgactagagaaatctgataatcttcttgagggtggagaagaggacaattttgctattgtgtttgcagctatgggagttaacatggagactgcacagtttttcaagcgtgactttgaggagaatggatcaatggagagagtgactcttttccttaatttggcaaatgatcctacaattgagcgtattatcactcctcgtattgctcttactactgctgaatatttggcttatgaatgtggcaagcacgttcttgtgatactcacagacatgagttcttatgctgatgctcttcgtgaggtatctgctgcccgagaagaagtgccgggtagacgtggttatcctggatatatgtatacagatcttgcaacaatttatgaacgagctgggagaattgaggggcgtaaaggctctattacacaaattccaattctaaccatgcccaatgatgatattacgcacccaactcctgatctaacaggatatatcactgaggggcagatatacatcgacaggcagctgtataatagacagatatacccaccaatcaacgtcctcccatcactatctcggttgatgaagagtgctattggtgagggaatgactagaagggatcattctgatgtgtccaaccagctctatgcaaattatgctattggaaaggatgtccaggcaatgaaagcagtggttggagaagaagcactttcatctgaggacctgctgtatctggaatttttggagaagtttgagaggaagtttgttgcccagggagcctatgacacccgcaatatattccagtcacttgatcttgcatggactttactccggattttccctcgtgaacttcttcatcgtataccagccaagactcttgaccagttttacagcagagatgctggtaat

SEQ ID NO: 286 - protein, 488 aa, Glycine max
MGVAENIPDMEEGTLEIGMEYRTVSGVAGPLVILDKVKGPKFQEIVNIRLGDGTTRRGQVLEVDGEKAVVQVFEGTSGIDNKFTTVQFTGEVLKTPVSLDMLGRIFNGSGKPIDNGPPILPEAYLDISGSSINPSERTYPEEMIQTGISTIDVMNSIARGQKIPLFSAAGLPHNEIAAQICRQAGLVKRLEKSDNLLEGGEEDNFAIVFAAMGVNMETAQFFKRDFEENGSMERVTLFLNLANDPTIERIITPRIALTTAEYLAYECGKHVLVILTDMSSYADALREVSAAREEVPGRRGYPGYMYTDLATIYERAGRIEGRKGSITQIPILTMPNDDITHPTPDLTGYITEGQIYIDRQLYNRQIYPPINVLPSLSRLMKSAIGEGMTRRDHSDVSNQLYANYAIGKDVQAMKAVVGEEALSSEDLLYLEFLEKFERKFVAQGAYDTRNIFQSLDLAWTLLRIFPRELLHRIPAKTLDQFYSRDAGN

SEQ ID NO: 287 - protein, 26 aa, Glycine max
FQEIVNIRLGDGTTRRGQVLEVDGEK

SEQ ID NO: 288 - protein, 12 aa, Glycine max
FTTVQFTGEVLK

SEQ ID NO: 289 - protein, 12 aa, Glycine max
TLDQFYSRDAGN

SEQ ID NO: 290 - DNA, 1407 bp, Glycine max
  source 1..1407: mol_type = other DNA
atggccaccgctgccgagaaactctccgctctcaaatccgccgtcgccggattgaacgaaatcagtgagaatgagaagaacggattcatcagcctcgtcggccgctatctcagtggcgaagcgcagcatgtggaatggagcaagatccagacgcctacggacgaagtggttgtgccttacgacactttggcgccaactcctgaaggttcttcggaggtgaagaatctattggacaagcttgtggtgttgaagctaaatggaggcttgggaacaactatgggttgcactggtcctaaatctgtaattgaagttcgtgatgggttgacatttctagatttaattgtcatccaaattgagaatctcaattccaaatatggaagcaatgttcctttgcttttgatgaattcattcaacactcatgatgacactcaaaagattgttgaaaaataccaaaactcaaatattgagattcatacttttaaccagagccagtatcctcgattggttgttgaggactttttgccattgccttccaaagggcatactgacaaggatggatggtaccctcctggccatggtgatgtcttcccatcattattgaacagtggcaaacttgatgcactattgtcacagggtaaagagtatgtgtttgttgccaattcggataacttgggagctatagttgacttgaaaatcttgaatcatttgatccagaacaagaatgaatactgtatggaggtgactcccaaaacattggctgatgtaaagggtggcactttgatttcttacgaaggaagggttcagctcctggaaattgcacaagtccccgatgaacatgtcaatgagttcaagtcaatagagaagttcaaaattttcaacacaaataatttgtgggtgaacttaaacgcagttaaaaggcttgttgaagctgatgctcttaagatggaaattattcccaatccaaaggaagttgacggaataaaagttcttcagctggaaactgcagctggtgctgcaataaggttctttgacaaggctattgggattaatgttcctcgatcacgattccttcctgtgaaggcaacttcagatttgcttcttgtccagtctgacctctacactttggaagacggatttgtcattcggaacaaagctagggaaaatcctgaaaacccttctattgaactgggaccagaatttaagaaggttagcaacttcttgggccgcttcaagtcaattcctagtatcgttgagcttgacagtctaaaagtggctggtgatgtatggtttggagctggtgttatcctcaaggggaaagtcagcattgtatcaaagcccggtgtaaagctggaagttcccgacggtgttgccattgtagacaaggaaattaatggcccagaggacctg

SEQ ID NO: 291 - protein, 469 aa, Glycine max
MATAAEKLSALKSAVAGLNEISENEKNGFISLVGRYLSGEAQHVEWSKIQTPTDEVVVPYDTLAPTPEGSSEVKNLLDKLVVLKLNGGLGTTMGCTGPKSVIEVRDGLTFLDLIVIQIENLNSKYGSNVPLLLMNSFNTHDDTQKIVEKYQNSNIEIHTFNQSQYPRLVVEDFLPLPSKGHTDKDGWYPPGHGDVFPSLLNSGKLDALLSQGKEYVFVANSDNLGAIVDLKILNHLIQNKNEYCMEVTPKTLADVKGGTLISYEGRVQLLEIAQVPDEHVNEFKSIEKFKIFNTNNLWVNLNAVKRLVEADALKMEIIPNPKEVDGIKVLQLETAAGAAIRFFDKAIGINVPRSRFLPVKATSDLLLVQSDLYTLEDGFVIRNKARENPENPSIELGPEFKKVSNFLGRFKSIPSIVELDSLKVAGDVWFGAGVILKGKVSIVSKPGVKLEVPDGVAIVDKEINGPEDL

SEQ ID NO: 292 - protein, 15 aa, Glycine max
AIGINVPRSRFLPVK

SEQ ID NO: 293 - protein, 17 aa, Glycine max
ARENPENPSIELGPEFK

SEQ ID NO: 294 - protein, 24 aa, Glycine max
ATSDLLLVQSDLYTLEDGFVIRNK

SEQ ID NO: 295 - protein, 8 aa, Glycine max
EINGPEDL

SEQ ID NO: 296 - protein, 18 aa, Glycine max
EYVFVANSDNLGAIVDLK

SEQ ID NO: 297 - protein, 9 aa, Glycine max
ILNHLIQNK

SEQ ID NO: 298 - protein, 26 aa, Glycine max
IQTPTDEVVVPYDTLAPTPEGSSEVK

SEQ ID NO: 299 - protein, 9 aa, Glycine max
LDALLSQGK

SEQ ID NO: 300 - protein, 9 aa, Glycine max
RLVEADALK

SEQ ID NO: 301 - protein, 14 aa, Glycine max
SAVAGLNEISENEK

SEQ ID NO: 302 - protein, 12 aa, Glycine max
SIPSIVELDSLK

SEQ ID NO: 303 - protein, 17 aa, Glycine max
VLQLETAAGAAIRFFDK

SEQ ID NO: 304 - protein, 9 aa, Glycine max
VSNFLGRFK

SEQ ID NO: 305 - DNA, 1353 bp, Glycine max
  source 1..1353: mol_type = other DNA
atgaccaccgccaccgagaagctctccgctctcaaatccgccgtcgccggattgaacgaaatcagtgagagtgagaagaacggattcatcagcctcgtcagccgctatctcagtggcgaagcgcagcatgtggaatggagcaagatccagacgcctacggacgaagtggttgtgccttacgacactttggcgccaactcctgatggttcttcggacgtgaagaatctattggacaagcttgtggtgttgaagctaaatggaggcttgggcacaactatgggttgcactggtcctaaatctgtaattgaagttcgtgatgggttgacatttctagatttaattgtgatccagattgagaatctcaattccaaatatggaagcaatgttcctttgcttttgatgaattcattcaacactcatgatgacactcaaaagattgttgaaaaataccaaaactccaatattgagattcatacttttaaccagggcatactgacaaggatggatggtaccctcctggcccatggagatgtctttccatcattattgaacagtggcaaacttgatgcactattgtcacagggtaaagagtatgtatttgttgccaattcagataacttgggagctatagttgacttgaaaatcttaaatcatttgatccagaacaagaatgaatactgtatggaggtgactcccaaaacattggctgatgtaaagggtggcactttgatttcttacgaaggaaggcttttggaaattgcacaagtcccagatgaacatgtcaatgagttcaagtcaatagagaagttcaaaattttcaacacaaataatttgtgggtgaacttaaatgcagttaaaaggcttgttgaagctgatgctcttaagatggaaattattcccaatccaaaggaagttgatggaataaaagttcttcagctggaaactgcagctggtgctgcaataaggttctttgacaaggctattgggattaatgttcctcgatcacgattccttcctgtgaaggcaacttcagatttgcttcttgtccagtctgacctctacactttggaagatggatttgtcattcggaacaaagctagggcaaatcctgaaaacccttctattgaacttgggccagaatttaagaaggttagcaacttcttgggccgcttcaagtcaattcccagtattgttgagcttgacagtctaaaagtggctggcaatgtatggtttggagctggtgttatcctcaagggaaaaatcagtatcgtggccaatcctggtgttaagctggaagttcccgatggtgctgtcatttcggataaggaaattaatggcccagaggacctcctg

SEQ ID NO: 306 - protein, 451 aa, Glycine max
MTTATEKLSALKSAVAGLNEISESEKNGFISLVSRYLSGEAQHVEWSKIQTPTDEVVVPYDTLAPTPDGSSDVKNLLDKLVVLKLNGGLGTTMGCTGPKSVIEVRDGLTFLDLIVIQIENLNSKYGSNVPLLLMNSFNTHDDTQKIVEKYQNSNIEIHTFNQGILTRMDGTLLAHGDVFPSLLNSGKLDALLSQGKEYVFVANSDNLGAIVDLKILNHLIQNKNEYCMEVTPKTLADVKGGTLISYEGRLLEIAQVPDEHVNEFKSIEKFKIFNTNNLWVNLNAVKRLVEADALKMEIIPNPKEVDGIKVLQLETAAGAAIRFFDKAIGINVPRSRFLPVKATSDLLLVQSDLYTLEDGFVIRNKARANPENPSIELGPEFKKVSNFLGRFKSIPSIVELDSLKVAGNVWFGAGVILKGKISIVANPGVKLEVPDGAVISDKEINGPEDLL

SEQ ID NO: 307 - protein, 15 aa, Glycine max
AIGINVPRSRFLPVK

SEQ ID NO: 308 - protein, 24 aa, Glycine max
ATSDLLLVQSDLYTLEDGFVIRNK

SEQ ID NO: 309 - protein, 18 aa, Glycine max
EYVFVANSDNLGAIVDLK

SEQ ID NO: 310 - protein, 9 aa, Glycine max
ILNHLIQNK

SEQ ID NO: 311 - protein, 26 aa, Glycine max
IQTPTDEVVVPYDTLAPTPDGSSDVK

SEQ ID NO: 312 - protein, 9 aa, Glycine max
LDALLSQGK

SEQ ID NO: 313 - protein, 12 aa, Glycine max
LEVPDGAVISDK

SEQ ID NO: 314 - protein, 9 aa, Glycine max
RLVEADALK

SEQ ID NO: 315 - protein, 14 aa, Glycine max
SAVAGLNEISESEK

SEQ ID NO: 316 - protein, 12 aa, Glycine max
SIPSIVELDSLK

SEQ ID NO: 317 - protein, 17 aa, Glycine max
VLQLETAAGAAIRFFDK

SEQ ID NO: 318 - protein, 9 aa, Glycine max
VSNFLGRFK

SEQ ID NO: 319 - DNA, 1332 bp, Glycine max
  source 1..1332: mol_type = other DNA
atggatgaggagtacgatgtgatcgtgttgggcacgggtctcaaggagtgcattctcagcggtcttctctccgtcgatggcctcaaggttctgcacatggacaggaatgactattacggaggagaatccacttcgctcaatcttattcagctttggaagagattcaggggagatgacaagcctcctccacatttgggagctagcagggactataacatcgatatgaaccctaagtttattatggctaatggcaatttggtgcgggttctcatacatactgatgttactaagtatctctctttcaaagctgtggatgggagctttgtgtttaataaaggaaaggttcataaggtgcccgcaaatgacatggaggccttgaagtctccacttatggggctatttgaaaaacgccgagcacgcaagtttttcatttatgtgcaaaattacaatgagagtgatcccaagacccatgaagggatggacttgacaagagtgactactaaagagttgatagcaaaatacggtcttgatgataacactatagacttcattggtcatgctttggcgcttcatagtgatgatcgctacttggctgagccagcactggacactgtgaagagaatgaagttatatgcagagtctcttgcacgctttcaaggagggtcaccttacatatatcctttgtatggtttaggagagcttccccaggcatttgcacggcttagtgctgtttatggtgggacatatatgttgaataagcctgagtgcaaggtagaatttgatgaggaaggaaaggttgttggtgtcacatctgaaggggaaactgcaaagtgcaaaaaagttgtttgtgatccatcatatttgcccggcaaggtgaggaaggttggtagggttgcaagagcaatagccataatgagtcacccaatccccagcactgatgattcccattcggtgcaaattattttaccacagaaacagttgggtcggaagtcagacatgtacctattttgttgttcatatactcacaatgttgctccaaagggaaaatttattgcatttgtatcaaccgaggcagagacagatcagccagcaattgaactaaaacctggaattgaccttctaggacctgttgatgagatattttttgatatctatgacagatatgaaccggtcaatgaacccactttggacaactgctttgtatcaacaagttatgatgctactactcactttgagtcgaccgtggatgatgttctcaacatgtatacattgataactggaaaggttattgacctgagtgtggatcttagtgctgctagtgctgcagaagag

SEQ ID NO: 320 - protein, 444 aa, Glycine max
MDEEYDVIVLGTGLKECILSGLLSVDGLKVLHMDRNDYYGGESTSLNLIQLWKRFRGDDKPPPHLGASRDYNIDMNPKFIMANGNLVRVLIHTDVTKYLSFKAVDGSFVFNKGKVHKVPANDMEALKSPLMGLFEKRRARKFFIYVQNYNESDPKTHEGMDLTRVTTKELIAKYGLDDNTIDFIGHALALHSDDRYLAEPALDTVKRMKLYAESLARFQGGSPYIYPLYGLGELPQAFARLSAVYGGTYMLNKPECKVEFDEEGKVVGVTSEGETAKCKKVVCDPSYLPGKVRKVGRVARAIAIMSHPIPSTDDSHSVQIILPQKQLGRKSDMYLFCCSYTHNVAPKGKFIAFVSTEAETDQPAIELKPGIDLLGPVDEIFFDIYDRYEPVNEPTLDNCFVSTSYDATTHFESTVDDVLNMYTLITGKVIDLSVDLSAASAAEE

SEQ ID NO: 321 - protein, 10 aa, Glycine max
AVDGSFVFNK

SEQ ID NO: 322 - protein, 14 aa, Glycine max
FFIYVQNYNESDPK

SEQ ID NO: 323 - protein, 8 aa, Glycine max
VEFDEEGK

SEQ ID NO: 324 - protein, 16 aa, Glycine max
VIDLSVDLSAASAAEE

SEQ ID NO: 325 - protein, 12 aa, Glycine max
VVGVTSEGETAK

SEQ ID NO: 326 - DNA, 1332 bp, Glycine max
  source 1..1332: mol_type = other DNA
atggatgaggagtacgatgtgatcgtgttgggcacgggtctcaaggagtgcattctcagcggtcttctctccgtcgatggcctcaaggttctgcatatggataggaacgactattacggaggagaatccacttcgctcaatcttattcagctttggaagagattcaggggagatgacaagcctcctccacatttgggcgctagcagggactacaacatcgatatgaaccccaagtttattatggctaatggcaatttggtgcgggttctcatacataccgatgttacaaagtatctctatttcaaagctgtggatgggagctatgtgtttaataaaggaaaggttcataaggtgcctgcaaacgacatggaggccttgaagtccccacttatggggctatttgaaaaacgccgggcacgcaagtttttcatttatgtgcaaaactacaacgagagcgatcccaagacccacgaggggatggacttgacaagagtgactactaaagagttgatagcaaaatacggccttgatgacaacactatagacttcattggtcatgctttggcgcttcatagcgatgatcgctacttggctgagccagcactggacaccgtgaagagaatgaagttatatgcagagtctcttgcacgctttcaaggagggtcaccttacatatatcctttgtatggtttaggagagcttccccaggcatttgcacggcttagtgctgtttatggtggaacatatatgttgaataagcctgagtgcaaggtagaatttgatgaggaaggaaaggttgttggtgttacatctgaaggggaaactgcaaagtgtaaaaaagttgtttgtgatccatcatatttgcctggcaaggtgaggaaggttggtagggttgcaagggcgatagccatcatgagccacccaatccccagcactgatgattcccattcagtgcaaattattttaccacagaaacagttgggtcggaagtcagacatgtacctattttgttgttcgtatactcacaatgttgctccaaagggaaaatttattgcatttgtatcaactgaggcagagacagatcagccggcaattggactgaaacctggaattgaccttctaggacctgttgatgagatattttttgatatctatgacagatatgaaccagtcaacgaacccactttggacaactgctttatatcaacaagttatgatgctactactcactttgagtcgactgtggatgacgttctcaacatgtatacattgataactggaaaggttattgacctcagtgtggatcttagtgctgctagtgctgcagaagag

SEQ ID NO: 327 - protein, 444 aa, Glycine max
MDEEYDVIVLGTGLKECILSGLLSVDGLKVLHMDRNDYYGGESTSLNLIQLWKRFRGDDKPPPHLGASRDYNIDMNPKFIMANGNLVRVLIHTDVTKYLYFKAVDGSYVFNKGKVHKVPANDMEALKSPLMGLFEKRRARKFFIYVQNYNESDPKTHEGMDLTRVTTKELIAKYGLDDNTIDFIGHALALHSDDRYLAEPALDTVKRMKLYAESLARFQGGSPYIYPLYGLGELPQAFARLSAVYGGTYMLNKPECKVEFDEEGKVVGVTSEGETAKCKKVVCDPSYLPGKVRKVGRVARAIAIMSHPIPSTDDSHSVQIILPQKQLGRKSDMYLFCCSYTHNVAPKGKFIAFVSTEAETDQPAIGLKPGIDLLGPVDEIFFDIYDRYEPVNEPTLDNCFISTSYDATTHFESTVDDVLNMYTLITGKVIDLSVDLSAASAAEE

SEQ ID NO: 328 - protein, 10 aa, Glycine max
AVDGSYVFNK

SEQ ID NO: 329 - protein, 14 aa, Glycine max
FFIYVQNYNESDPK

SEQ ID NO: 330 - protein, 8 aa, Glycine max
VEFDEEGK

SEQ ID NO: 331 - protein, 16 aa, Glycine max
VIDLSVDLSAASAAEE

SEQ ID NO: 332 - protein, 12 aa, Glycine max
VVGVTSEGETAK

SEQ ID NO: 333 - DNA, 1512 bp, Glycine max
  source 1..1512: mol_type = other DNA
atggcgatgagaaccaaactttctttagctatctttttcttttttcttttagccttattttcaaacctagcctttggcaaatgtaaagaaaccgaggtagaagaagaagaccctgagctcgtaacatgcaaacaccaatgccaacagccacagcaatacactgagggtgacaagcgggtatgcttgcaaagatgtgacaggtatcatcgtatgaagcaagagcgagagaaacaaatccaagaggaaactcgtgagaagaaagaggaggaaagccgtgaaagggaagaagaacaacaagagcagcatgaggaacaagatgaaaatccctacatttttgaggaagataaggattttgagaccagagtcgaaactgaaggtggcagaattcgggttctcaagaagttcactgaaaaatccaagcttcttcagggcattgagaacttccgtttggccattttggaagctagagcacacacgttcgtgtctcctcgccactttgattccgaggttgtttttttcaacatcaaggggcgagccgtacttgggttggtgagtgaaagtgaaacagaaaaaatcacccttgaacctggggacatgatacacataccagctggcaccccactgtacatcgttaacagagatgagaatgataagctcttccttgccatgctccatatacctgtctctgtctctactcctggaaaatttgaggaatttttcgggcctggaggacgagacccagaatcggtcctctcagcattcagctggaatgtgctgcaagctgcgctccaaaccccaaaaggaaagttagaaaagctttttgatcaacagaacgagggaagtattttcgcaataagcagagaacaggtgcgtgcgttggcccctaccaagaaaagctcttggtggccattcggtggcgaatccaagcctcaattcaatattttcagcaagcgtcccactatctccaacggatatggtcgtttaactgaagttggtcctgacgatgatgaaaagagttggcttcaaagactcaatctcatgcttacctttaccaacatcacccagagatctatgagtactattcactacaactcacatgcaacgaagatagcactggtgatcgatggcagagggcatcttcaaatatcatgtccacacatgtcatcaaggtcgtcacactcgaagcatgataagagtagcccttcataccatagaatcagttccgatttgaagcctggaatggtgtttgttgtccctcccggtcatcccttcgtcaccatagcttccaataaagagaatctcctcatgatttgcttcgaggttaacgctcgagacaacaagaagtttacatttgcagggaaggacaacattgtgagttctctggacaacgttgctaaggagcttgcctttaactatccttctgagatggtgaacggagtattcgaccgaaaggagagtttctttttcccttttgagttgccgagagaggagcgtggtcgtcgcgctgatgcg

SEQ ID NO: 334 - protein, 504 aa, Glycine max
MAMRTKLSLAIFFFFLLALFSNLAFGKCKETEVEEEDPELVTCKHQCQQPQQYTEGDKRVCLQRCDRYHRMKQEREKQIQEETREKKEEESREREEEQQEQHEEQDENPYIFEEDKDFETRVETEGGRIRVLKKFTEKSKLLQGIENFRLAILEARAHTFVSPRHFDSEVVFFNIKGRAVLGLVSESETEKITLEPGDMIHIPAGTPLYIVNRDENDKLFLAMLHIPVSVSTPGKFEEFFGPGGRDPESVLSAFSWNVLQAALQTPKGKLEKLFDQQNEGSIFAISREQVRALAPTKKSSWWPFGGESKPQFNIFSKRPTISNGYGRLTEVGPDDDEKSWLQRLNLMLTFTNITQRSMSTIHYNSHATKIALVIDGRGHLQISCPHMSSRSSHSKHDKSSPSYHRISSDLKPGMVFVVPPGHPFVTIASNKENLLMICFEVNARDNKKFTFAGKDNIVSSLDNVAKELAFNYPSEMVNGVFDRKESFFFPFELPREERGRRADA

SEQ ID NO: 335 - protein, 17 aa, Glycine max
DFETRVETEGGRIRVLK

SEQ ID NO: 336 - protein, 12 aa, Glycine max
DNIVSSLDNVAK

SEQ ID NO: 337 - protein, 15 aa, Glycine max
GRAVLGLVSESETEK

SEQ ID NO: 338 - protein, 25 aa, Glycine max
LFDQQNEGSIFAISREQVRALAPTK

SEQ ID NO: 339 - protein, 21 aa, Glycine max
RPTISNGYGRLTEVGPDDDEK

SEQ ID NO: 340 - DNA, 1467 bp, Glycine max
  source 1..1467: mol_type = other DNA
atggcgaccagagccaagctttctttagctatcttccttttctttcttttagccttgatttcaaacctagccttgggcaaacttaaagaaaccgaggtcgaagaagatcccgagctcgtaacatgcaaacaccagtgccaacagcaacggcaatacactgagagtgacaagcgaacatgcttgcaacaatgtgacagtatgaagcaagagcgagagaaacaagtcgaagaggaaactcgcgagaaggaagaagaacatcaagagcagcatgaggaggagcaagaccaaaatccctacgtttttgaagaagataaggatttttcgaccagagtcgaaacagaaggtggcagcattcgggttctcaagaagttcactgagaaatccaagcttcttcaaggcaatgagaatttccgtttggccatcttagaagctagagcacacacgttcgtgtccccacgccactttgattccgaggttgtcttgttcaacattaaggggagagccgtacttgggttggtgagggaaagtgaaacagaaaaaatcaccctagaacctggagacatgatacacataccagcaggcacaccactgtacatcgttaacagagatgagaatgagaagctcctccttgccatgctccatatacctgtctctactcctggaaaatttgaggaatttttcgggcctggaggacgagacccagaatcggtcctctcagcattcagctggaatgtgctgcaagctgcgctccaaaccccaaaaggaaagttagaaaggctttttaatcaacagaacgagggaagtattttcaaaataagcagagaacgggtgcgtgcgttggcccccaccaagaaaagctcttggtggccattcggcggcgaatccaaggctcaattcaatattttcagcaagcgtcccactttctccaacggatatggccgtttaactgaagttggtcctgatgatgaaaagagttggcttcaaagactcaacctcatgcttacctttaccaacatcacccagagatctatgagtactattcactacaactcacatgcaacgaagatagcactggtgatggatggtagagggcatcttcaaatatcatgtccacacatgtcatcaaggtcagactcaaagcatgataagagtagcccctcataccatagaatcagtgcggacttgaagcctggaatggtgtttgttgtccctcctggtcatcccttcgtcactatagcttccaataaagagaatctcctcataatttgcttcgaggttaacgttcgagacaacaagaagtttacgtttgcagggaaggacaacattgtgagctctctggacaacgtagctaaggagctggcctttaactatccttctgagatggtgaacggagtcttcgaaagaaaggagagtctctttttccccttcgagttgccgagcgaggagcgtggtcgtcgcgctgttgcg

SEQ ID NO: 341 - protein, 489 aa, Glycine max
MATRAKLSLAIFLFFLLALISNLALGKLKETEVEEDPELVTCKHQCQQQRQYTESDKRTCLQQCDSMKQEREKQVEEETREKEEEHQEQHEEEQDQNPYVFEEDKDFSTRVETEGGSIRVLKKFTEKSKLLQGNENFRLAILEARAHTFVSPRHFDSEVVLFNIKGRAVLGLVRESETEKITLEPGDMIHIPAGTPLYIVNRDENEKLLLAMLHIPVSTPGKFEEFFGPGGRDPESVLSAFSWNVLQAALQTPKGKLERLFNQQNEGSIFKISRERVRALAPTKKSSWWPFGGESKAQFNIFSKRPTFSNGYGRLTEVGPDDEKSWLQRLNLMLTFTNITQRSMSTIHYNSHATKIALVMDGRGHLQISCPHMSSRSDSKHDKSSPSYHRISADLKPGMVFVVPPGHPFVTIASNKENLLIICFEVNVRDNKKFTFAGKDNIVSSLDNVAKELAFNYPSEMVNGVFERKESLFFPFELPSEERGRRAVA

SEQ ID NO: 342 - protein, 8 aa, Glycine max
AQFNIFSK

SEQ ID NO: 343 - protein, 17 aa, Glycine max
DFSTRVETEGGSIRVLK

SEQ ID NO: 344 - protein, 12 aa, Glycine max
DNIVSSLDNVAK

SEQ ID NO: 345 - protein, 15 aa, Glycine max
GRAVLGLVRESETEK

SEQ ID NO: 346 - protein, 20 aa, Glycine max
RPTFSNGYGRLTEVGPDDEK

SEQ ID NO: 347 - DNA, 1266 bp, Glycine max
  source 1..1266: mol_type = other DNA
atggtgagaggattgctcaacaagctcgtctctcgctccctctcggtcgccggaaaatggcagcacaatcaactccgccgcctcaacatccacgagtaccagggtgctgaattgatgagcaaacacggagtcaacgttccgagaggcgtggctgtttcctctgttgaagaggccagaaaggtcatcaaggaccttttccctaatgaaaatgagttggtggttaagagtcaaattttagccggcggacgaggtttgggaactttcaaaagcggccttaagggaggagtgcacattgttaagactgaccaggttgaagacatagctgggaagatgcttggacagatactagttaccaaacagactggtcctcagggaaaaatagttagcaaggtttacttgtgtgaaaagctgtcacttgtaaatgagatgtactttgctataacactggatcgtacgagtgctggtccaattataattgcttgtagtaagggaggaaccagcattgaagaccttgcagagaaatttccggacatgattataaaggtacctgttgatgtttttgaaggtattactgatgaaggtgctgcaaaggttgttgatggtttggctctcaaagtggctgatagaaataagtcgattgaacaagtgaagaatttgtataaactttttgttgattgtgactgcactcttttggaaatcaatcccatagcggagacagctgataaccaattggtggctgctgatgctaagttgaattttgatgataatgctgcatatcgccagaaagagatattttctctccgtgatacaacacaagaggaccctcgagaggtgactgctgcaaaggcagatttaaattatattgggttagatggagaaattggctgcatggtgaatggagcagggttagcaatggccacaatggatataattaaattgcatgggggtactcctgccaattttctggatgtaggtggcaatgcctctgaaaaccaggttgttgaggcatttaaaatattgactgctgatgacaaagtgaaggcaattttggttaacatttttggtggtataatgaagtgtgatgttatagcaagtggaatagtgaatgctgcaaaagaggttgcactgaaagtaccagttgtagttcgtctggaaggcaccaatgttgatcaaggaaaaagaatattgaaggaaagtggtatggcattaataacggctgaagatttagatgatgcagctcagaaagcagtgaaagctgcttacaaa

SEQ ID NO: 348 - protein, 422 aa, Glycine max
MVRGLLNKLVSRSLSVAGKWQHNQLRRLNIHEYQGAELMSKHGVNVPRGVAVSSVEEARKVIKDLFPNENELVVKSQILAGGRGLGTFKSGLKGGVHIVKTDQVEDIAGKMLGQILVTKQTGPQGKIVSKVYLCEKLSLVNEMYFAITLDRTSAGPIIIACSKGGTSIEDLAEKFPDMIIKVPVDVFEGITDEGAAKVVDGLALKVADRNKSIEQVKNLYKLFVDCDCTLLEINPIAETADNQLVAADAKLNFDDNAAYRQKEIFSLRDTTQEDPREVTAAKADLNYIGLDGEIGCMVNGAGLAMATMDIIKLHGGTPANFLDVGGNASENQVVEAFKILTADDKVKAILVNIFGGIMKCDVIASGIVNAAKEVALKVPVVVRLEGTNVDQGKRILKESGMALITAEDLDDAAQKAVKAAYK

SEQ ID NO: 349 - protein, 12 aa, Glycine max
DLFPNENELVVK

SEQ ID NO: 350 - protein, 20 aa, Glycine max
EIFSLRDTTQEDPREVTAAK

SEQ ID NO: 351 - protein, 26 aa, Glycine max
LHGGTPANFLDVGGNASENQVVEAFK

SEQ ID NO: 352 - protein, 12 aa, Glycine max
LNFDDNAAYRQK

SEQ ID NO: 353 - protein, 14 aa, Glycine max
SQILAGGRGLGTFK

SEQ ID NO: 354 - protein, 10 aa, Glycine max
TDQVEDIAGK

SEQ ID NO: 355 - protein, 16 aa, Glycine max
VPVVVRLEGTNVDQGK

SEQ ID NO: 356 - protein, 8 aa, Glycine max
VVDGLALK

SEQ ID NO: 357 - DNA, 1266 bp, Glycine max
  source 1..1266: mol_type = other DNA
atggtgaggggattgctcaacaagctcgtctctcgctccctctccgtcgccggaaaatggcagcacaaccaactccgccgcctcaacatccacgagtaccagggcgctgaattgatgagcaaacacggagttaatgttccgagaggcgtggctgtttcctctgttgaagaggtcagaaaggccatcaaggaccttttccctaatgaaaatgagttggtggttaagagtcaaattttagccggtggaagaggtttgggaactttcaaaagcggccttaagggaggagtgcacattgttaagactgaccaggttgaagacatagctgggaagatgcttgggcagatactagttaccaaacagactggtcctcagggaaaaatagttagcaaggtttacttgtgtgaaaagctgtcactagtaaatgagatgtactttgctataacactggatcgtacgagtgctggtccaattataattgcttgtagtaagggaggaaccagcattgaagacctagcagagaaatttccggacatgattataaaggtacctgttgatgtttttgaaggaattactgatgaagatgctgcaaaggttgttgatggtttggctctcaaagtggctgatagaaataagtcgattgaacaagtgaagaatttgtataaactttttgttgattgtgactgcacccttttggaaatcaatcccattgcggagacagctgataaccaattggtggctgctgatgctaagttgaattttgacgataatgctgcatatcgccagaaagagatatttgctctccgtgatacaacacaagaggaccctcgagaggtgactgctgcaaaggcagacttaaattatattgggttagatggagaaataggctgcatggtgaatggagcagggttagcaatggccacaatggatataattaaattgcatgggggtactcctgccaattttttggatgtaggtggcaatgcctctgaaggccaggtggttgaggcatttaaaatattgactgctgatgacaaagtgaaggcaattttggttaacatttttgggggtataatgaagtgtgatgttatagcaagtggaatagtgaatgctgcaaaagaggttgcactgaaagtacccgtagtagttcgtctggaaggcaccaatgttgatcatggaaaaagaattttgaaggaaagtggtatggcgttgataacggctgaagatttagatgatgctgctcagaaagcagtgaaagctgcttacaaa

SEQ ID NO: 358 - protein, 422 aa, Glycine max
MVRGLLNKLVSRSLSVAGKWQHNQLRRLNIHEYQGAELMSKHGVNVPRGVAVSSVEEVRKAIKDLFPNENELVVKSQILAGGRGLGTFKSGLKGGVHIVKTDQVEDIAGKMLGQILVTKQTGPQGKIVSKVYLCEKLSLVNEMYFAITLDRTSAGPIIIACSKGGTSIEDLAEKFPDMIIKVPVDVFEGITDEDAAKVVDGLALKVADRNKSIEQVKNLYKLFVDCDCTLLEINPIAETADNQLVAADAKLNFDDNAAYRQKEIFALRDTTQEDPREVTAAKADLNYIGLDGEIGCMVNGAGLAMATMDIIKLHGGTPANFLDVGGNASEGQVVEAFKILTADDKVKAILVNIFGGIMKCDVIASGIVNAAKEVALKVPVVVRLEGTNVDHGKRILKESGMALITAEDLDDAAQKAVKAAYK

SEQ ID NO: 359 - protein, 12 aa, Glycine max
DLFPNENELVVK

SEQ ID NO: 360 - protein, 20 aa, Glycine max
EIFALRDTTQEDPREVTAAK

SEQ ID NO: 361 - protein, 26 aa, Glycine max
LHGGTPANFLDVGGNASEGQVVEAFK

SEQ ID NO: 362 - protein, 12 aa, Glycine max
LNFDDNAAYRQK

SEQ ID NO: 363 - protein, 14 aa, Glycine max
SQILAGGRGLGTFK

SEQ ID NO: 364 - protein, 10 aa, Glycine max
TDQVEDIAGK

SEQ ID NO: 365 - protein, 8 aa, Glycine max
VVDGLALK

SEQ ID NO: 366 - DNA, 1203 bp, Glycine max
  source 1..1203: mol_type = other DNA
atggcgactaagaggagcgtgggaacgttgaaggagggtgatttgaaggggaagagggtattcgtgagggtcgatctgaacgtgcctttggatgacaaccttaacatcaccgatgacactagagtccgtgctgctgttcccaccatcaagtacttgactggttatggtgctaaagtgatcctttctagccatttgggacgtccaaaaggtgtaacacctaaatacagtttgaagcctcttgtgccaaggctgtctcaacttcttggaattgaggttacgatggccaatgactctattggggaggaagttgaaaagttggttacacaacttccagaaggtggtgttttgcttctagagaatgtgaggttctacaaggaggaagagaagaatgatcctgaatttgcaaagaagttggcttctctagctgatctctatgtgaatgatgcatttggcactgcccacagagctcatgcttctacagaaggagttgccaaatacttgaagccctctgttgcaggattcctaatgcagaaggagcttgattatctagttggggctgtgtcaaaccccaagagaccatttgctgctattgtcggtggatcgaaggtgtcttccaagattggagttattgaatccttgttggagaaagttaatgttctcttgcttggtggaggaatgatctttaccttttacaaggctcagggttattcagttgggtcatctcttgtggaggaagacaagcttagccttgcaactacacttcttgaaaaggccaaggctaaaggggtttctttgttgcttccaactgatgttgtcatagcagacaagtttgctgctgatgctaacagcaagactgtgccggcatcaagcatcccagatgggtggatggggttggatattggtcctgattccatcaagacatttggtgaagcattggatacgactcagaccattatttggaatggaccaatgggtgtttttgagtttgataagtttgccacaggaacagaggctatagccaagaaactggcagagctgagtgggaagggagtgacaaccatcattggaggaggtgactccgttgccgctgtggagaaggttggacttgcagacaagatgagccacatctccactggtggtggtgccagcttagagcttcttgagggaaagcaactccctggtgtccttgctcttgatgatgct

SEQ ID NO: 367 - protein, 401 aa, Glycine max
MATKRSVGTLKEGDLKGKRVFVRVDLNVPLDDNLNITDDTRVRAAVPTIKYLTGYGAKVILSSHLGRPKGVTPKYSLKPLVPRLSQLLGIEVTMANDSIGEEVEKLVTQLPEGGVLLLENVRFYKEEEKNDPEFAKKLASLADLYVNDAFGTAHRAHASTEGVAKYLKPSVAGFLMQKELDYLVGAVSNPKRPFAAIVGGSKVSSKIGVIESLLEKVNVLLLGGGMIFTFYKAQGYSVGSSLVEEDKLSLATTLLEKAKAKGVSLLLPTDVVIADKFAADANSKTVPASSIPDGWMGLDIGPDSIKTFGEALDTTQTIIWNGPMGVFEFDKFATGTEAIAKKLAELSGKGVTTIIGGGDSVAAVEKVGLADKMSHISTGGGASLELLEGKQLPGVLALDDA

SEQ ID NO: 368 - protein, 15 aa, Glycine max
AQGYSVGSSLVEEDK

SEQ ID NO: 369 - protein, 13 aa, Glycine max
ELDYLVGAVSNPK

SEQ ID NO: 370 - protein, 8 aa, Glycine max
FAADANSK

SEQ ID NO: 371 - protein, 10 aa, Glycine max
FATGTEAIAK

SEQ ID NO: 372 - protein, 15 aa, Glycine max
GVSLLLPTDVVIADK

SEQ ID NO: 373 - protein, 17 aa, Glycine max
GVTTIIGGGDSVAAVEK

SEQ ID NO: 374 - protein, 10 aa, Glycine max
IGVIESLLEK

SEQ ID NO: 375 - protein, 28 aa, Glycine max
LASLADLYVNDAFGTAHRAHASTEGVAK

SEQ ID NO: 376 - protein, 10 aa, Glycine max
LSLATTLLEK

SEQ ID NO: 377 - protein, 20 aa, Glycine max
LVTQLPEGGVLLLENVRFYK

SEQ ID NO: 378 - protein, 7 aa, Glycine max
NDPEFAK

SEQ ID NO: 379 - protein, 11 aa, Glycine max
QLPGVLALDDA

SEQ ID NO: 380 - protein, 11 aa, Glycine max
RPFAAIVGGSK

SEQ ID NO: 381 - protein, 11 aa, Glycine max
VILSSHLGRPK

SEQ ID NO: 382 - protein, 8 aa, Glycine max
YLTGYGAK

SEQ ID NO: 383 - DNA, 1455 bp, Glycine max
  source 1..1455: mol_type = other DNA
atggcctcagccgcatcccccaccactttctctctcctccactccgccaccccttcctcctcctccaccgccacctccgcctccccctccccctcgcgcgtgcagctcacctcctccctccgcgcccgcccgctgcgccgcctgggcttcgccgcggcggacccgctgctcgcggcgagtgtagcggcgcgcgtggcggcggtgagaggaggaggaaagggcgcgaggggggtggtgtcgatggcgaagaagagcgtgggggacctgagcgcggcggacctgaaggggaagaaggtcttcgtcagggccgacctcaacgtcccgctcgacgacaaccagaacatcaccgacgacaccagaatccgcgccgccattcccaccatcaaacatctcatccaaaacggcgccaaagtcattctctccagccacttggggcggccgaagggtgtcactcccaaatacagcttggcgcctcttgtaccccggctttctgagctcattggtatccagattgtcaaggccgaggacagtattggtccagaagtagaaaagttggtggcttctcttccagatggaggtgttctccttctagaaaatgtgaggttttacaaggaggaagaaaagaatgaccccgagcatgcaaaaaagcttgcctctctggcagatctgtatgtgaatgatgcatttggtactgctcacagggcacatgcatcaacagagggtgttactaaatacctgaagccatctgttgctggttttcttttgcaaaaggaacttgattaccttgttggggcagtatcaaaccccaaaaggccatttgctgccattgttggtggttccaaagtctcatctaaaattggagtgattgagtcacttttagaaaaagttgacatccttcttcttggtggaggaatgatcttcacattttacaaggcacaaggtctttcagtgggttcatcccttgtagaagaagataagttggatcttgctacatcactacttgcaaaagccaaggaaaagggggtgtctctcttgttaccaagtgatgtggtgattgcagacaaatttgctcctgatgctaatagcaaggtcgtgccagcatctgccatccctgatggctggatgggattggatattggtccagattctgttaaatcattcagtgaagcattggatactacccaaaccatcatatggaatggaccaatgggagtgtttgagtttgacaagtttgctgttggtacagaggctattgcaaagaagctggctgacctcagtggaaagggggtgaccacaattattggaggaggagattctgttgcagctgtggagaaagtaggagttgctagtgtcatgagccacatatctactggtggtggtgccagtttggagttattggaaggcaaagagcttccaggagtccttgctcttgatgaagccgtcccagtcgctgtg

SEQ ID NO: 384 - protein, 485 aa, Glycine max
MASAASPTTFSLLHSATPSSSSTATSASPSPSRVQLTSSLRARPLRRLGFAAADPLLAASVAARVAAVRGGGKGARGVVSMAKKSVGDLSAADLKGKKVFVRADLNVPLDDNQNITDDTRIRAAIPTIKHLIQNGAKVILSSHLGRPKGVTPKYSLAPLVPRLSELIGIQIVKAEDSIGPEVEKLVASLPDGGVLLLENVRFYKEEEKNDPEHAKKLASLADLYVNDAFGTAHRAHASTEGVTKYLKPSVAGFLLQKELDYLVGAVSNPKRPFAAIVGGSKVSSKIGVIESLLEKVDILLLGGGMIFTFYKAQGLSVGSSLVEEDKLDLATSLLAKAKEKGVSLLLPSDVVIADKFAPDANSKVVPASAIPDGWMGLDIGPDSVKSFSEALDTTQTIIWNGPMGVFEFDKFAVGTEAIAKKLADLSGKGVTTIIGGGDSVAAVEKVGVASVMSHISTGGGASLELLEGKELPGVLALDEAVPVAV

SEQ ID NO: 385 - protein, 13 aa, Glycine max
ELDYLVGAVSNPK

SEQ ID NO: 386 - protein, 16 aa, Glycine max
ELPGVLALDEAVPVAV

SEQ ID NO: 387 - protein, 10 aa, Glycine max
FAVGTEAIAK

SEQ ID NO: 388 - protein, 15 aa, Glycine max
GVSLLLPSDVVIADK

SEQ ID NO: 389 - protein, 17 aa, Glycine max
GVTTIIGGGDSVAAVEK

SEQ ID NO: 390 - protein, 10 aa, Glycine max
IGVIESLLEK

SEQ ID NO: 391 - protein, 11 aa, Glycine max
RPFAAIVGGSK

SEQ ID NO: 392 - protein, 11 aa, Glycine max
VILSSHLGRPK

SEQ ID NO: 393 - DNA, 1203 bp, Glycine max
  source 1..1203: mol_type = other DNA
atggcgactaagaggagcgtgggaacgttgaaggagggtgatttgaaagggaagagggtgttcgtgagggtcgatctgaacgtgcctttggatgacaaccttaacatcaccgatgacactagagtccgtgctgctgttcccaccatcaagtacttaactggtcatggagccaaagtgatcctttctagccacttgggacgtcctaaaggtgtaacacctaaatacagtttgaagcctcttgtgccaaggctgtctcaacttctcggaattgaggttacgatggcaaatgactctattggggaggaagttgagaagttggttgcacaacttccagaaggtggtgttttgcttctagagaatgtgaggttctataaggaggaagagaagaatgaccctgagtttgcaaagaagttggcttctcttgctgatctctacgtgaatgatgctttcggcactgcccacagagctcatgcttctacagaaggagtggccaaatacttgaagccctctgttgcaggattccttatgcagaaggaacttgactatctagttggagctgtgtcaaaccccaagagaccatttgctgctatcgttggtgggtcaaaggtgtcttccaagattggagttattgagtccttgttggagaaagttaacgttctcttgcttggtggaggaatgatctttaccttttacaaggctcagggttattcagttggttcatctcttgtggaggaagacaagcttagccttgcaactacacttctcgaaaaggccaaggctaaaggggtttctttgttgcttccaactgatgtggtcatagcagacaagtttgctgctgatgctaacagcaagactgtgccagcatcaagcatcccagacgggtggatgggattagatattggtcccgattccatcaagacatttggtgaagcattggataagacccagaccgtcatttggaatggaccaatgggtgtttttgagtttgataagtttgccacaggaacagaggccatagccaagaaactggcagagctgagtgggaagggagtaacgaccatcattggaggaggtgactctgttgccgctgtggagaaggttggtcttgcagacaagatgagccacatctccactggtggtggtgccagcttagagcttcttgaggggaagcaactccctggtgtccttgcccttgatgatgct

SEQ ID NO: 394 - protein, 401 aa, Glycine max
MATKRSVGTLKEGDLKGKRVFVRVDLNVPLDDNLNITDDTRVRAAVPTIKYLTGHGAKVILSSHLGRPKGVTPKYSLKPLVPRLSQLLGIEVTMANDSIGEEVEKLVAQLPEGGVLLLENVRFYKEEEKNDPEFAKKLASLADLYVNDAFGTAHRAHASTEGVAKYLKPSVAGFLMQKELDYLVGAVSNPKRPFAAIVGGSKVSSKIGVIESLLEKVNVLLLGGGMIFTFYKAQGYSVGSSLVEEDKLSLATTLLEKAKAKGVSLLLPTDVVIADKFAADANSKTVPASSIPDGWMGLDIGPDSIKTFGEALDKTQTVIWNGPMGVFEFDKFATGTEAIAKKLAELSGKGVTTIIGGGDSVAAVEKVGLADKMSHISTGGGASLELLEGKQLPGVLALDDA

SEQ ID NO: 395 - protein, 15 aa, Glycine max
AQGYSVGSSLVEEDK

SEQ ID NO: 396 - protein, 13 aa, Glycine max
ELDYLVGAVSNPK

SEQ ID NO: 397 - protein, 8 aa, Glycine max
FAADANSK

SEQ ID NO: 398 - protein, 10 aa, Glycine max
FATGTEAIAK

SEQ ID NO: 399 - protein, 15 aa, Glycine max
GVSLLLPTDVVIADK

SEQ ID NO: 400 - protein, 17 aa, Glycine max
GVTTIIGGGDSVAAVEK

SEQ ID NO: 401 - protein, 10 aa, Glycine max
IGVIESLLEK

SEQ ID NO: 402 - protein, 28 aa, Glycine max
LASLADLYVNDAFGTAHRAHASTEGVAK

SEQ ID NO: 403 - protein, 10 aa, Glycine max
LSLATTLLEK

SEQ ID NO: 404 - protein, 20 aa, Glycine max
LVAQLPEGGVLLLENVRFYK

SEQ ID NO: 405 - protein, 7 aa, Glycine max
NDPEFAK

SEQ ID NO: 406 - protein, 11 aa, Glycine max
QLPGVLALDDA

SEQ ID NO: 407 - protein, 11 aa, Glycine max
RPFAAIVGGSK

SEQ ID NO: 408 - protein, 8 aa, Glycine max
TFGEALDK

SEQ ID NO: 409 - protein, 11 aa, Glycine max
VILSSHLGRPK

SEQ ID NO: 410 - DNA, 1143 bp, Glycine max
  source 1..1143: mol_type = other DNA
atgtcgtcgagcacagttggtcaagtcatcaaatgcaaagctgcggtttcatgggaggcagggaagccacttgtgattgaagaagtagaggtggcaccaccacaagctggtgaagtgcgtttgaagatcctctacacctctctttgccacactgatgtttacttctgggaagccaagggccaaactccattgtttcctcgtatatttggtcatgaagctggagggattgtggagagcgtaggtgagggtgtgactcatctcaaaccaggggaccatgccctcccagtgtttaccggggagtgtggagaatgccctcattgcaagtcagaggagagcaacatgtgtgatcttctcaggatcaacactgataggggtgtcatgatccatgacagccaaacaagattttctattaagggacaacctatttaccattttgttggtacctccacattcagtgaatacactgttgttcatgccggatgtgttgccaaagtcaaccctgctgccccacttgacaaaatttgtgttctcagttgtggaatttgcacaggtcttggggctactatcaatgttgcaaaaccaaaacctggttcttctgttgccatttttggacttggagctgttggccttgctgctgctgaaggggcaaggatttctggtgcatcaagaattattggggttgatttagtttctagcagatttgaagaagctaagaagtttggggtcaatgaatttgtgaacccaaaagaccatgacaagcctgtacaagaggtgattgctgcaatgacgaatgggggtgtggatcgtgctgttgaatgtactggcagcatccaagccatgatctcggcattcgaatgcgtccacgatggttggggtgttgctgtacttgttggagtgccaaacaaagatgatgcattcaaaactcatccagtgaatttcttgaacgagagaactctaaagggtaccttctacggaaactacaaaccccgcactgatcttccttctgttgtggagaagtacatgaatggggaattggaactggagaaattcatcactcacacagttccattctcagagattaacaaggcttttgattacatgctgaaaggggagtccatcagatgcatcattcgaatgggggag

SEQ ID NO: 411 - protein, 381 aa, Glycine max
MSSSTVGQVIKCKAAVSWEAGKPLVIEEVEVAPPQAGEVRLKILYTSLCHTDVYFWEAKGQTPLFPRIFGHEAGGIVESVGEGVTHLKPGDHALPVFTGECGECPHCKSEESNMCDLLRINTDRGVMIHDSQTRFSIKGQPIYHFVGTSTFSEYTVVHAGCVAKVNPAAPLDKICVLSCGICTGLGATINVAKPKPGSSVAIFGLGAVGLAAAEGARISGASRIIGVDLVSSRFEEAKKFGVNEFVNPKDHDKPVQEVIAAMTNGGVDRAVECTGSIQAMISAFECVHDGWGVAVLVGVPNKDDAFKTHPVNFLNERTLKGTFYGNYKPRTDLPSVVEKYMNGELELEKFITHTVPFSEINKAFDYMLKGESIRCIIRMGE

SEQ ID NO: 412 - protein, 10 aa, Glycine max
FGVNEFVNPK

SEQ ID NO: 413 - protein, 13 aa, Glycine max
FITHTVPFSEINK

SEQ ID NO: 414 - protein, 19 aa, Glycine max
GTFYGNYKPRTDLPSVVEK

SEQ ID NO: 415 - protein, 13 aa, Glycine max
THPVNFLNERTLK

SEQ ID NO: 416 - protein, 9 aa, Glycine max
VNPAAPLDK

SEQ ID NO: 417 - DNA, 1113 bp, Glycine max
  source 1..1113: mol_type = other DNA
atgcgtgcagctgcggtggcatgggaagcagggaagccactgtcaatagaaaccatagaagtggcaccaccccagaaaggtgaagtgcgtttgaggatcctcttcaactccctttgccgtagtgatgtttattggtgggatgctaaggaccagactcctctgtttcctcgtatcttaggccacgaagcttcagggattgtggagagcgtgggcgagggtgtgacacatctgaaaccaggtgaccatgcactcccaatattcactggggagtgtggtgaatgcacttattgtaagtctgaagagagcaacctgtgtgagctactcaggatcaacactgacaggggtgttatgctgagtgatggaaaaacaaggttctccaaaaatggccaacccatttaccacttcgttggaacctccactttcagtgaatacactgttctccatgaaggatgtgtagccaagattaaccctaatgccccacttgacaaagttgctattgtcagctgtggattctgcacaggttttggggctactgtgaatgttgcaaaaccaaagcctaataatactgttgctgtctttggcttaggagctgttggtcttgctgcctgtgaaggggcaagggtttctggtgcatctagaatcatcggggttgatttacttcccaatcgatttgaacaagccaaaaaatttggggtcactgattttgtaaacccgaaagatcacaacaaaccagtccaagaggtcattgctgaaatgactaatggaggagtagatcgtgctattgaatgcactggaagcatccaagcttcaatctcagcatttgaatgcactcatgatggttggggtactgctgtccttgttggtgtgccaaagaaggatgttgaattcaaaactaatcctatgaagttcatggaagggagaactcttaagggtaccttctatggtcactacagaccccgcactgacattcctggtgttgtggagaagtacctcaacaaggagttggaactagacaaatttatcactcactctgtgccattctcaaagatcaacactgcatttgatcttatgctgaaaggggagggcataaggtgcctcatctgcatggaagag

SEQ ID NO: 418 - protein, 371 aa, Glycine max
MRAAAVAWEAGKPLSIETIEVAPPQKGEVRLRILFNSLCRSDVYWWDAKDQTPLFPRILGHEASGIVESVGEGVTHLKPGDHALPIFTGECGECTYCKSEESNLCELLRINTDRGVMLSDGKTRFSKNGQPIYHFVGTSTFSEYTVLHEGCVAKINPNAPLDKVAIVSCGFCTGFGATVNVAKPKPNNTVAVFGLGAVGLAACEGARVSGASRIIGVDLLPNRFEQAKKFGVTDFVNPKDHNKPVQEVIAEMTNGGVDRAIECTGSIQASISAFECTHDGWGTAVLVGVPKKDVEFKTNPMKFMEGRTLKGTFYGHYRPRTDIPGVVEKYLNKELELDKFITHSVPFSKINTAFDLMLKGEGIRCLICMEE

SEQ ID NO: 419 - protein, 19 aa, Glycine max
GTFYGHYRPRTDIPGVVEK

SEQ ID NO: 420 - protein, 9 aa, Glycine max
INPNAPLDK

SEQ ID NO: 421 - DNA, 1140 bp, Glycine max
  source 1..1140: mol_type = other DNA
atgtctagcaccgttggccagaccatcaagtgcaaagctgcgattgcatgggaggctgggaagccgctggtgattgaagaagtggaggttgcgccaccgcaagccggtgaagtccgtttgaagatcctctacacctctctttgccacactgatgtttacttctgggatgcgaagggccagactccattgtttcctcgcatttttggccatgaagcttcggggattgtggagagcgtaggcgagggtgtgactcatctgaaaccaggtgaccatgccctccctgtgttcacaggagagtgtggagattgcgcccattgcaagtcagaggagagcaacatgtgtgagctactcaggatcaacaccgataggggtgtcatgatccatgatggccaatcaaggttctctaagaatggacaacccatacaccatttcttgggaacctctacattcagtgaatacactgttgtccatgctggatgtgttgcaaagatcaaccctgctgctccacttgacaaagtttgtgttctcagttgtggaatttgcacaggttttggtgctactgtaaatgtagcaaaaccaaaacctggttcctctgttgccatatttggacttggagctgttggccttgcggctgctgaaggtgcaagagtttcaggtgcttcaagaatcattggagttgatttagtttctgcccgatttgaagaagctaagaagtttggggttaatgagtttgtgaacccaaaggatcatgataaacctgtgcaacaggtaattgctgaaatgaccaatggaggtgtggatcgggctgttgaatgtactggcagcatccaagccatggtctcagcattcgaatgcgtccacgatggttggggtcttgctgtacttgttggtgtgcctagtaaagatgatgcattcaaaactgctcctattaatttcctgaacgagaggactcttaagggcaccttttatggcaactacaaaccacgcaccgatcttccatctgttgtcgagaagtacatgagtggggagctagaagtggacaaattcatcactcacacagttccattctcagagatcaacaaagcttttgatttaatgctgaagggacagtccattaggtgtatcatccgcatgcaagag

SEQ ID NO: 422 - protein, 380 aa, Glycine max
MSSTVGQTIKCKAAIAWEAGKPLVIEEVEVAPPQAGEVRLKILYTSLCHTDVYFWDAKGQTPLFPRIFGHEASGIVESVGEGVTHLKPGDHALPVFTGECGDCAHCKSEESNMCELLRINTDRGVMIHDGQSRFSKNGQPIHHFLGTSTFSEYTVVHAGCVAKINPAAPLDKVCVLSCGICTGFGATVNVAKPKPGSSVAIFGLGAVGLAAAEGARVSGASRIIGVDLVSARFEEAKKFGVNEFVNPKDHDKPVQQVIAEMTNGGVDRAVECTGSIQAMVSAFECVHDGWGLAVLVGVPSKDDAFKTAPINFLNERTLKGTFYGNYKPRTDLPSVVEKYMSGELEVDKFITHTVPFSEINKAFDLMLKGQSIRCIIRMQE

SEQ ID NO: 423 - protein, 10 aa, Glycine max
FGVNEFVNPK

SEQ ID NO: 424 - protein, 13 aa, Glycine max
FITHTVPFSEINK

SEQ ID NO: 425 - protein, 19 aa, Glycine max
GTFYGNYKPRTDLPSVVEK

SEQ ID NO: 426 - protein, 9 aa, Glycine max
INPAAPLDK

SEQ ID NO: 427 - protein, 13 aa, Glycine max
TAPINFLNERTLK

SEQ ID NO: 428 - DNA, 927 bp, Glycine max
  source 1..927: mol_type = other DNA
atggcgtcaagacagcaattcaaggaagacagagccgaggcagctgcaaaactcgcagcgaaagacatcggcgacatcaacagaacaaacgagcgcgacgaggggtacgacttgcacgacgaagccaacttcaagcaagcacgagctgaagcagctgcaaagctggcggccaaggatctggaggacgctaacagattaagagacaacactttcaacacatggaaagagcaacatcagcatgatgagaacaaacccggtgtgatagggtccatgtttagagccgccaaggaagccgtggttgggaagcctcaccacgaggaagtttatgcaaaggaaacaaagatgggtgaatattcagattatgcgactcagaaggcgagggaaacgaagcagaaggctggggagtatacagactatgcggctcagaaagcaaaagaaaccaaagactatgctgctgagaaggctaagaatgctaaagataccactgtgcagaaagctggagagtacaaggattataccgctgagaaggctaagaatgctaaagataccactgtgcagaaagctggagaatacaaggattataccgctgagaaggcaattgaagggaaggacagtgcagtggggaagcttggggagctaaaggaatctgctgctgatgctgctaaaagggctatgggtttcttcaccggtggcaacaaagaccaaactagagaggaggaggaggaaacgaggcggagtatgcaggaggtgagggtgcaagataaggagtatggaacgggacgtggtactggagagaagttggtgataaaaatggaagagtcgcggccaggagcggtggcggatgcgctcaaagcagcgaatagggccatggacggcgatatggaggaggaaggcgtgcttcatgtggagcgtcgtagggagaaaatg

SEQ ID NO: 429 - protein, 309 aa, Glycine max
MASRQQFKEDRAEAAAKLAAKDIGDINRTNERDEGYDLHDEANFKQARAEAAAKLAAKDLEDANRLRDNTFNTWKEQHQHDENKPGVIGSMFRAAKEAVVGKPHHEEVYAKETKMGEYSDYATQKARETKQKAGEYTDYAAQKAKETKDYAAEKAKNAKDTTVQKAGEYKDYTAEKAKNAKDTTVQKAGEYKDYTAEKAIEGKDSAVGKLGELKESAADAAKRAMGFFTGGNKDQTREEEEETRRSMQEVRVQDKEYGTGRGTGEKLVIKMEESRPGAVADALKAANRAMDGDMEEEGVLHVERRREKM

SEQ ID NO: 430 - protein, 11 aa, Glycine max
AGEYTDYAAQK

SEQ ID NO: 431 - protein, 24 aa, Glycine max
DIGDINRTNERDEGYDLHDEANFK

SEQ ID NO: 432 - protein, 17 aa, Glycine max
DLEDANRLRDNTFNTWK

SEQ ID NO: 433 - protein, 15 aa, Glycine max
EAVVGKPHHEEVYAK

SEQ ID NO: 434 - DNA, 1104 bp, Glycine max
  source 1..1104: mol_type = other DNA
atgggcaaggtcaagatcggaatcaacggatttggaagaattggccgtttggtagccagagtggctctgcagagagacgatgttgaactcgttgccgttaacgaccctttcatcaccaccgattacatgacatacatgtttaaatacgacagtgttcatggacactggaagcatcacgatgtcaccgttaaggacgagaagacccttctcttcggtgacaagccagtcactatttttggacacaggtttttatttttcactttaatttttcttactgtttatgattgttgtgtcctgcatgtaaacgttgagtgttgttttctctgtgaactcagaaaccctgaagagatcccatgggggtcaactggagctgacatcattgttgagtccaccggagttttcaccgataaggacaaggccgccgcacatttgaagggtggtgcaaagaaggttattatttctgcccccagtaaggatgcccccatgtttgttgttggtgtcaacgagcacgagtacaagccagagcttgatattatttccaatgctagctgcacaaccaactgccttgccccacttgccaaggttatcaatgacaggtttggcattgttgagggtttgatgaccactgttcattccatcaccgctacccagaagactgttgatggaccatcagccaaggactggagaggtggaagagctgcttcatttaacatcattcctagcagcactggagctgccaaggctgttgggaaagtcctccctgctttgaatggaaaattgactggtatggcattccgtgttcccaccgtggatgtctctgttgttgacctcacagtgaggctggagaaagaagcttcctacgatgaaattaaaaatgctatcaaggaggaatcagagggcaagttgaagggaattcttggttacactgaagatgatgtggtctccactgactttatcggcgatagcagatcaagtatttttgatgcaaaggctggaattgcattgaataagaactttgtgaagcttgtttcttggtacgacaacgagtggggatacagctcacgtgtcattgatcttcttgtattcgttgccaagaagtctctt

SEQ ID NO: 435 - protein, 368 aa, Glycine max
MGKVKIGINGFGRIGRLVARVALQRDDVELVAVNDPFITTDYMTYMFKYDSVHGHWKHHDVTVKDEKTLLFGDKPVTIFGHRFLFFTLIFLTVYDCCVLHVNVECCFLCELRNPEEIPWGSTGADIIVESTGVFTDKDKAAAHLKGGAKKVIISAPSKDAPMFVVGVNEHEYKPELDIISNASCTTNCLAPLAKVINDRFGIVEGLMTTVHSITATQKTVDGPSAKDWRGGRAASFNIIPSSTGAAKAVGKVLPALNGKLTGMAFRVPTVDVSVVDLTVRLEKEASYDEIKNAIKEESEGKLKGILGYTEDDVVSTDFIGDSRSSIFDAKAGIALNKNFVKLVSWYDNEWGYSSRVIDLLVFVAKKSL

SEQ ID NO: 436 - protein, 8 aa, Glycine max
EASYDEIK

SEQ ID NO: 437 - protein, 27 aa, Glycine max
GILGYTEDDVVSTDFIGDSRSSIFDAK

SEQ ID NO: 438 - protein, 8 aa, Glycine max
VIISAPSK

SEQ ID NO: 439 - protein, 8 aa, Glycine max
VLPALNGK

SEQ ID NO: 440 - DNA, 1014 bp, Glycine max
  source 1..1014: mol_type = other DNA
atgggcaaggtcaagatcggaatcaacggatttggaagaattggccgtttggttgccagagtcgctctgcaaagagacgatgtggagctcgttgccgttaacgaccctttcattaccaccgattacatgacatacatgtttaaatacgacagtgttcacggacactggaagcatcacgatgtcaccgttaaggacgagaagacccttctcttcggtgacaagccagtcactgtttttggacacaggaaccctgaagagatcccatgggggtcaactggagctgatatcattgttgagtccaccggagttttcaccgataaggacaaggccgccgcacatttgaagggtggtgcgaagaaggttattatttctgcccccagtaaggatgcccccatgtttgttgttggtgttaacgagcacgagtacaagccagagcttgatattatttccaatgctagctgcacaaccaactgccttgctccacttgctaaggttattaatgacaggtttggcattgttgagggtttgatgaccactgttcattccatcactgccacccagaagactgttgatgggccatcagccaaagactggagaggtggaagagctgcttcatttaacatcattcctagcagcactggagctgccaaggctgttggaaaagtccttcctgctttgaacggaaaattgactggtatggcattccgtgttcccaccgtggatgtctctgttgttgacctcacagtgaggctggagaaggaagcctcctacgatgaaattaaaaatgctatcaaggaggaatcagagggcaagttgaagggaattcttggttacactgaagatgatgtggtctccaccgactttgtgggcgatagcagatcaagtatttttgatgcaaaggctggaattgcattgaataagaactttgtgaagcttgtctcttggtatgacaacgagtggggatacagctcacgtgtcattgacctgcttgtattcgttgccaagaagtctttt

SEQ ID NO: 441 - protein, 338 aa, Glycine max
MGKVKIGINGFGRIGRLVARVALQRDDVELVAVNDPFITTDYMTYMFKYDSVHGHWKHHDVTVKDEKTLLFGDKPVTVFGHRNPEEIPWGSTGADIIVESTGVFTDKDKAAAHLKGGAKKVIISAPSKDAPMFVVGVNEHEYKPELDIISNASCTTNCLAPLAKVINDRFGIVEGLMTTVHSITATQKTVDGPSAKDWRGGRAASFNIIPSSTGAAKAVGKVLPALNGKLTGMAFRVPTVDVSVVDLTVRLEKEASYDEIKNAIKEESEGKLKGILGYTEDDVVSTDFVGDSRSSIFDAKAGIALNKNFVKLVSWYDNEWGYSSRVIDLLVFVAKKSF

SEQ ID NO: 442 - protein, 8 aa, Glycine max
EASYDEIK

SEQ ID NO: 443 - protein, 27 aa, Glycine max
GILGYTEDDVVSTDFVGDSRSSIFDAK

SEQ ID NO: 444 - protein, 8 aa, Glycine max
VIISAPSK

SEQ ID NO: 445 - protein, 8 aa, Glycine max
VLPALNGK

SEQ ID NO: 446 - DNA, 1020 bp, Glycine max
  source 1..1020: mol_type = other DNA
atggcatcagataagaagattaggatcggcatcaacggatttggaaggattggccgtttggtggccagggttgcccttcaaaggaatgatgttgaacttgttgctgtcaacgatcctttcatcaccactgattacatgacatacatgttcaagtacgacaccgttcatggccagtggaagcattttgatgtgaaggtgaaggactctaagacccttctctttggtgagaagcccgtcaccgtttttggaatcaggaaccctgaggaaattccatggggtgaggttggtgctgattatgttgttgagtcaactggtgtattcactgacaaggacaaggctgctgctcacttgaagggtggtgccaagaaggttgtcatctctgccccaagcaaagatgcacccatgtttgttgttggtgttaacgagaaggagtacaagccagagcttgacattgtttccaatgctagctgcactaccaattgtcttgctcccctcgccaaggttatcaacgaccgatttggaattgtggagggtcttatgaccactgttcatgccatcacagctactcagaagactgttgatggtccatcaaacaaggactggaggggtggaagagctgcttccttcaatataattcccagcagcactggagctgctaaggctgtaggaaaagttcttccagcactgaatggtaaattgactggcatgtccttccgagtccccactgtagatgtttcagttgttgacctcactgtcaggctagagaagccagcaacctatgagcaaatcaaatctgctatcaaggaggaatcagagggcaaattgaagggtattttgggttacaccgaagatgatgttgtgtctactgattttgttggtgataacaggtctagcatatttgacgccaaggccggaatttctttgaacgacaactttgtaaaacttgtttcctggtatgacaacgaatggggttacagttcccgtgtgattgatttgattgtccacattgcatccgtggct

SEQ ID NO: 447 - protein, 340 aa, Glycine max
MASDKKIRIGINGFGRIGRLVARVALQRNDVELVAVNDPFITTDYMTYMFKYDTVHGQWKHFDVKVKDSKTLLFGEKPVTVFGIRNPEEIPWGEVGADYVVESTGVFTDKDKAAAHLKGGAKKVVISAPSKDAPMFVVGVNEKEYKPELDIVSNASCTTNCLAPLAKVINDRFGIVEGLMTTVHAITATQKTVDGPSNKDWRGGRAASFNIIPSSTGAAKAVGKVLPALNGKLTGMSFRVPTVDVSVVDLTVRLEKPATYEQIKSAIKEESEGKLKGILGYTEDDVVSTDFVGDNRSSIFDAKAGISLNDNFVKLVSWYDNEWGYSSRVIDLIVHIASVA

SEQ ID NO: 448 - protein, 11 aa, Glycine max
AGISLNDNFVK

SEQ ID NO: 449 - protein, 27 aa, Glycine max
GILGYTEDDVVSTDFVGDNRSSIFDAK

SEQ ID NO: 450 - protein, 8 aa, Glycine max
VLPALNGK

SEQ ID NO: 451 - protein, 8 aa, Glycine max
VVISAPSK

SEQ ID NO: 452 - DNA, 1050 bp, Glycine max
  source 1..1050: mol_type = other DNA
atggtgacctcgtcgcaaaagattctggttaccggcggtgccggtttcataggcacccacaccgtggtccagcttctcaaaggaggcttcagtgtttcaataatcgacaatttcgataactccgtcgtggaagcagtggaccgcgtccgccaagtggtgggccctcagctttctcagaacctggaattcacccagggcgatctcaggaatagggatgacttggagaaactcttctccagaacaacttttgatgccgtgatccactttgctggcctgaaagcggttgctgaaagcgtttccaagccccgtcgctattttgattttaatttggtcggcaccatcaacctctaccaagttatggcaaagtataattgcagaaagatggttttctcatcatctgcaaccgtttatggccaacctgaaaagataccgtgtgaggaggatttcaggttacaagctatgaatccctatggacggaccaagcttttcctggaagaaattgcccgagatattcagaaagctgaaccagaatggaagatcatattactgaggtacttcaatccggttggggctcatgaaagtggtaaactcggtgaagatcccaagggcatcccaaataacctcatgccttacattcagcaagtagccgttggaagattgccggaactcaatgtatatgggcatgattatcccacaagggatggctctgcgatccgggactatatccatgtgatggacttagcagatggtcatattgctgccttgcgaaagctcttcacaacagagaacataggttgtactgcttacaacctgggaactggtcgtggaacatccgtgcttgaaatggttacagcatttgaaaaggcgtctggcaagaaaattccagtaaaattatgtccaagaagaccgggagatgctactgaggtttatgcatctacagagagagctgagaaagaacttggatggaaagcaaaatatggtgtggatgagatgtgcagggaccaatggaattgggcaaagaacaatccccagggttacacggggaagcct

SEQ ID NO: 453 - protein, 350 aa, Glycine max
MVTSSQKILVTGGAGFIGTHTVVQLLKGGFSVSIIDNFDNSVVEAVDRVRQVVGPQLSQNLEFTQGDLRNRDDLEKLFSRTTFDAVIHFAGLKAVAESVSKPRRYFDFNLVGTINLYQVMAKYNCRKMVFSSSATVYGQPEKIPCEEDFRLQAMNPYGRTKLFLEEIARDIQKAEPEWKIILLRYFNPVGAHESGKLGEDPKGIPNNLMPYIQQVAVGRLPELNVYGHDYPTRDGSAIRDYIHVMDLADGHIAALRKLFTTENIGCTAYNLGTGRGTSVLEMVTAFEKASGKKIPVKLCPRRPGDATEVYASTERAEKELGWKAKYGVDEMCRDQWNWAKNNPQGYTGKP

SEQ ID NO: 454 - protein, 12 aa, Glycine max
LFLEEIARDIQK

SEQ ID NO: 455 - DNA, 1050 bp, Glycine max
  source 1..1050: mol_type = other DNA
atggtgtcttcctcccaacacattctggtcaccggtggtgccggtttcattggcacccacaccgtcgttcagcttctcaaagctggcttcagcgtttcaataatcgacaatttcgataactccgtcatggaagcagtggaccgcgtccgccaagtggttggccctctgctttctcagaacctccaattcacccagggcgatctccggaatagggatgacttggagaaactcttctccaaaacaacatttgatgccgtgatccactttgctggcttgaaagcggttgctgaaagcgttgcgaagccccgtcgctattttgattttaatttggttggcaccatcaacctctacgaatttatggcaaagtataattgcaaaaagatggttttctcatcatctgcaaccgtttatggccaacctgaaaagataccgtgtgaggaggatttcaagttacaagctatgaatccctatggacggaccaagcttttcctggaagaaattgcccgagatattcagaaagctgaaccagaatggaagatcatattactgagatacttcaatccagttggggctcatgaaagtggcaaactcggtgaagatcccaagggcatcccaaataacctcatgccttacattcagcaagtagctgttggaagattgactgaactcaatgtatacggtcatgattatccaacgagggatggctctgcgatccgggactatatccatgtgatggacttggcagatggccatattgctgccctgcgaaagctcttcacaacggagaacataggttgtactgcttacaacctgggaactggtcgtggaacatctgtgcttgaaatggttgcagcatttgaaaaggcttctggcaagaaaattccagtaaaattatgtccaagaagaccgggagatgcgactgaggtttatgcatctacagagagagctgagaaagaacttggttggaaggcaaactatggtgtggaggagatgtgcagggaccaatggaattgggcaaagaacaatccctggggttacgcggggaagcct

SEQ ID NO: 456 - protein, 350 aa, Glycine max; identical to SEQ ID NO: 453

SEQ ID NO: 457 - protein, 12 aa, Glycine max
LFLEEIARDIQK

SEQ ID NO: 458 - protein, 13 aa, Glycine max
TTFDAVIHFAGLK

SEQ ID NO: 459 - DNA, 1341 bp, Glycine max
  source 1..1341: mol_type = other DNA
atgggtaaggaaaagactcacatcaacattgtcgtcattggacatgtcgactctgggaagtcaactaccactggtcatttgatctacaagcttggaggtattgacaagcgtgtgattgagaggttcgagaaggaggctgctgagatgaacaagaggtcattcaagtatgcctgggtgcttgacaagctcaaggctgagcgcgaaagaggaattaccattgatattgctttgtggaagtttgaaaccaccaagtactactgcacagtcatcgatgcccccggacatcgtgactttatcaagaacatgattactggtacctcccaggctgattgtgctgtccttatcattgactccaccactggtggttttgaagctggtatttctaaggatggacagactcgtgagcatgctcttcttgctttcacccttggtgtgaagcagatgatctgctgctgtaacaagatggatgccactaccccgaagtactctaaggctaggtatgatgaaattgtgaaggaagtctcttcctacttgaagaaggttggttacaacccagacaagattccatttgtgcccatctctggtttcgagggtgacaacatgattgagaggtccaccaaccttgactggtacaagggaccaactctccttgaggctcttgaccaaatcaatgagcccaagaggccctcagacaagccactcaggcttccattgcaggatgtctacaaaattggtggtattggtaccgtgccagtgggacgtgttgagactggtgtcgtgaagcccggtatggtggtgacttttggtcccactgggctgacaactgaggttaagtctgttgagatgcaccatgaggctctcacagaggctcttccaggtgacaatgttggctttaatgtgaagaatgttgcagtcaaggatctcaagcgtggttttgttgcatccaactccaaggatgaccctgccaaggaagctgccaacttcacatctcaagtcattatcatgaaccaccctggccagattggaaatggatacgcaccggtgcttgattgccacacttctcacattgctgtcaagttttctgaaatcttgaccaagattgacaggcgatctggtaaggagcttgagaaggagcccaaatttttgaagaatggtgatgcaggtatggttaagatgattccaaccaagcccatggtggttgaaactttctctgagtatcctccccttggtcgttttgctgtgagggacatgcgtcaaactgtggctgtcggagtcatcaagagtgttgagaagaaagaccccaccggagccaaggtcaccaaggccgctgccaagaagaag

SEQ ID NO: 460 - protein, 447 aa, Glycine max
MGKEKTHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIVKEVSSYLKKVGYNPDKIPFVPISGFEGDNMIERSTNLDWYKGPTLLEALDQINEPKRPSDKPLRLPLQDVYKIGGIGTVPVGRVETGVVKPGMVVTFGPTGLTTEVKSVEMHHEALTEALPGDNVGFNVKNVAVKDLKRGFVASNSKDDPAKEAANFTSQVIIMNHPGQIGNGYAPVLDCHTSHIAVKFSEILTKIDRRSGKELEKEPKFLKNGDAGMVKMIPTKPMVVETFSEYPPLGRFAVRDMRQTVAVGVIKSVEKKDPTGAKVTKAAAKKK

SEQ ID NO: 461 - protein, 8 aa, Glycine max
ARYDEIVK

SEQ ID NO: 462 - protein, 17 aa, Glycine max
DGQTREHALLAFTLGVK

SEQ ID NO: 463 - protein, 7 aa, Glycine max
EVSSYLK

SEQ ID NO: 464 - protein, 7 aa, Glycine max
FSEILTK

SEQ ID NO: 465 - protein, 10 aa, Glycine max
STTTGHLIYK

SEQ ID NO: 466 - protein, 15 aa, Glycine max
THINIVVIGHVDSGK

SEQ ID NO: 467 - DNA, 1341 bp, Glycine max
  source 1..1341: mol_type = other DNA
atgggtaaagagaaggttcacattaacattgtggtcattggccatgttgactctgggaagtcaaccaccactggccacttgatttacaagcttggaggtattgacaagcgtgttattgagaggtttgagaaagaagctgctgagatgaacaagaggtcattcaaatacgcctgggtgcttgacaagcttaaggctgagcgtgagagaggaattaccattgatattgcattatggaagtttgagaccaccaaatactactgcactgttattgatgctccaggacatcgtgatttcatcaagaacatgattactggaacttcccaggctgattgtgctgtccttatcattgattccacaactggtggttttgaagctggtatttccaaagatggacagactcgcgagcatgctctacttgccttcacccttggtgtcaagcaaatgatctgttgctgtaacaagatggatgccactaccccaaagtactctaaggccaggtatgaagaaattgtgaaggaagtttcttcctacttgaagaaggtaggttacaacccagacaagattccatttgttcccatctcaggttttgagggtgacaacatgattgagaggtccaccaatcttgattggtacaagggaccaacccttctcgaggctcttgaccagatcaatgagcccaagagaccctcagacaagcctttgaggcttcccttgcaggatgtctacaagattggtggtattggaacagtgccagtgggacgagttgaaactggtgtcttgaagcccggtatggtggtgacttttgctcccactggcctgacaactgaagttaagtctgtggagatgcaccatgaagctctccaggaggcccttccaggtgacaacgtaggatttaatgtgaagaatgttgcagtcaaggatctcaagcgtggttttgttgcctcaaactctaaggatgatcctgccaaggaggctgccaacttcacatcccaagtcattataatgaaccatcctggccaaattggaaatggttatgctcctgtgcttgactgccacacttctcacattgccgtcaagtttgccgaacttgtgaccaagattgacaggcgatctggtaaggagattgagaaggagcccaaattcttgaaaaatggagatgcaggttatgttaagatgattcccaccaagcctatggtggtcgaaactttctccgagtatcctcctcttggtcgttttgccgtgagggacatgcgacaaactgtggctgttggagtcatcaagagtgtggagaagaaggacccaactggagccaaggtcaccaaggctgcacagaagaagaag

SEQ ID NO: 468 - protein, 447 aa, Glycine max
MGKEKVHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYEEIVKEVSSYLKKVGYNPDKIPFVPISGFEGDNMIERSTNLDWYKGPTLLEALDQINEPKRPSDKPLRLPLQDVYKIGGIGTVPVGRVETGVLKPGMVVTFAPTGLTTEVKSVEMHHEALQEALPGDNVGFNVKNVAVKDLKRGFVASNSKDDPAKEAANFTSQVIIMNHPGQIGNGYAPVLDCHTSHIAVKFAELVTKIDRRSGKEIEKEPKFLKNGDAGYVKMIPTKPMVVETFSEYPPLGRFAVRDMRQTVAVGVIKSVEKKDPTGAKVTKAAQKKK

SEQ ID NO: 469 - protein, 17 aa, Glycine max
DGQTREHALLAFTLGVK

SEQ ID NO: 470 - protein, 7 aa, Glycine max
EVSSYLK

SEQ ID NO: 471 - protein, 10 aa, Glycine max
STTTGHLIYK

SEQ ID NO: 472 - DNA, 1344 bp, Glycine max
  source 1..1344: mol_type = other DNA
atgggtaaggaaaaggttcacatcagtattgtggtcattggccatgtcgactctgggaaatccactaccactggtcacctgatttacaagcttggaggcattgacaagcgtgttattgagaggtttgagaaggaagctgctgagatgaacaagaggtctttcaagtatgcctgggtgctggacaaacttaaggctgagcgtgaaagaggaatcaccattgatattgctttgtggaagtttgaaacaacaaagtattattgcacagttattgatgcgcctggacatagggatttcattaagaatatgattactgggacatcccaagctgactgtgctgttcttatcattgattcgaccactggtggttttgaagctggtatttccaaggatggacagactcgtgaacatgctctgctttcattcacccttggtgtgaaacagatgatttgctgctgtaacaaaatggatgctactacacccaagtattccaaggccaggtatgatgaaattgtgaaggaagtctcttcctacttgaagaaagtaggatacaaccctgacaagattccttttgttcctatctctggttttgagggagacaacatgattgagaggtccacaaaccttgactggtacaagggtccaactctgcttgatgcacttgaccagattagtgagcccaagaggccctctgacaagcccctcaggcttccccttcaggatgtgtacaagattggaggtattggaactgtgccagtgggacgtgttgagaccggtgtcttgaagcctggaatggtggtgacttttgcaccaactggactgacaactgaagtcaagtctgtggagatgcaccatgaatctcttacagaggcacatcctggtgacaatgtgggattcaatgttaagaatgttgctgttaaggatttgaagcgtggttatgttgcctcaaactcaaaggatgaccctgcaaaggaggctgctaacttcacagcccaagtcatcatcatgaaccaccctggtcagattggaaatggctatgcccctgtcctcgactgccacacttctcacattgctgtcaagtttgctgaactcatgaccaagattgacaggcgatccggcaaagagcttgagaaggagcccaagtttttgaagaacggtgatgctggttttgttaagatgattccaaccaaacccatggttgttgaaactttctccgagtatcctccacttggtaggtttgctgttagggacatgcgtcaaactgttgctgtgggagtcatcaagaacgttgagaagaaggatcctaccggagccaaggtcaccaaggctgcccagaagaagaagtga

SEQ ID NO: 473 - protein, 447 aa, Glycine max
MGKEKVHISIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLSFTLGVKQMICCCNKMDATTPKYSKARYDEIVKEVSSYLKKVGYNPDKIPFVPISGFEGDNMIERSTNLDWYKGPTLLDALDQISEPKRPSDKPLRLPLQDVYKIGGIGTVPVGRVETGVLKPGMVVTFAPTGLTTEVKSVEMHHESLTEAHPGDNVGFNVKNVAVKDLKRGYVASNSKDDPAKEAANFTAQVIIMNHPGQIGNGYAPVLDCHTSHIAVKFAELMTKIDRRSGKELEKEPKFLKNGDAGFVKMIPTKPMVVETFSEYPPLGRFAVRDMRQTVAVGVIKNVEKKDPTGAKVTKAAQKKK

SEQ ID NO: 474 - protein, 8 aa, Glycine max
ARYDEIVK

SEQ ID NO: 475 - protein, 17 aa, Glycine max
DGQTREHALLSFTLGVK

SEQ ID NO: 476 - protein, 7 aa, Glycine max
EVSSYLK

SEQ ID NO: 477 - protein, 10 aa, Glycine max
STTTGHLIYK

SEQ ID NO: 478 - DNA, 882 bp, Glycine max
  source 1..882: mol_type = other DNA
atggcttccggtgaacagaaattccctcctcaacaacaacaaacacagcctgggaaggagcatgctatgactccagtaccccaattcactagccctgactacaagccttcaaataaacttcaagggaagattgcattagtcactgggggtgattctgggattggacgagcggtgtgtaacttgtttgccttagaaggtgctaccgtggccttcacgtatgtgaaggggcatgaggacaaggacgcgagggacacattggaaatgatcaagagagcaaagacttcggatgccaaggatccaatggcaatagcatctgatttgggttacgatgagaactgcaagagggtggttgatgaggtcgtgagtgcttatggttgtattgacattctggtcaacaatgcagctgagcagtacgagtgtggaaccgtggaggacatagacgagcctaggcttgagagggtctttcgtacaaatatcttctcctatttcttcatggcgaggcatgccttgaagcacatgaaggaaggaagcagcattatcaacacgacatcagtgaatgcatacaagggacatgcgaaactattggactacacgtccaccaagggggcaattgtggcctatacaaggggtcttgcccttcagctggtgagtaagggaattcgggttaatggggtggctccagggcccatttggacccctttgataccagcctctttcaaggaggaagaaacggcccaatttggagcgcaggtcccaatgaagagagctggtcaacctattgaggttgctccttcctatgtttttcttgcttccaaccaatgctcctcttacataactggacaagtccttcaccccaatggtggaaccgttgtgaatggttaa

SEQ ID NO: 479 - protein, 293 aa, Glycine max
MASGEQKFPPQQQQTQPGKEHAMTPVPQFTSPDYKPSNKLQGKIALVTGGDSGIGRAVCNLFALEGATVAFTYVKGHEDKDARDTLEMIKRAKTSDAKDPMAIASDLGYDENCKRVVDEVVSAYGCIDILVNNAAEQYECGTVEDIDEPRLERVFRTNIFSYFFMARHALKHMKEGSSIINTTSVNAYKGHAKLLDYTSTKGAIVAYTRGLALQLVSKGIRVNGVAPGPIWTPLIPASFKEEETAQFGAQVPMKRAGQPIEVAPSYVFLASNQCSSYITGQVLHPNGGTVVNG

SEQ ID NO: 480 - protein, 15 aa, Glycine max
EGSSIINTTSVNAYK

SEQ ID NO: 481 - protein, 12 aa, Glycine max
FPPQQQQTQPGK

SEQ ID NO: 482 - protein, 17 aa, Glycine max
GAIVAYTRGLALQLVSK

SEQ ID NO: 483 - protein, 8 aa, Glycine max
LLDYTSTK

SEQ ID NO: 484 - DNA, 882 bp, Glycine max
  source 1..882: mol_type = other DNA
atggcaagcaataaggagtctaagttcccagcacagagccagaaaactcagccaggaaaagaacatgtaatgaatccactcccacaagccacaaatcctgatcacaaggccgccaataaactccagggaaaggtggcgttggtgacaggaggtgactcaggaattggcagagcggtttgcctgtgtttcgcaaaagagggtgcaaccgtggcctttacatacgtaaagggccatgaggacagggataaagatgatactctgaagatgctgcttgaagctaagacaagtggtgcagacaatccattggcaatagcagcggatattggctttgatgagaactgcaaacaggtcattgaccttgttgtcaaagaatatggccgccttgatgttctggtcaacaatgcagctgagcagcatttgacaaactctgttgaggaaatcacacaacagcagcttgagagagtcttcggaaccaacatcttttctcagttctttttggtcaagcatgctctgaagcacatgaaagaagggagctgcatcataaactctacttcagttaatgcatacaatgggaatccagaagcgttggactacactgctaccaagggagcaattgtggccttcaccagaggtctttctcagcagctagcgagtaggggaattagggtgaatggtgtggcacctggcccagtttggacgccaatacaaccagcttcaaagcctgctgagatgattcagaacttggggtgtgaggtgccaatgaaccgcgtggctcagccttgtgagattgcaccatgttatttgttcttggcaacttgtcaggactcttcctactttactggccaagtcctccatccaaatggtgggatggtcgtcaacgct

SEQ ID NO: 485 - protein, 294 aa, Glycine max
MASNKESKFPAQSQKTQPGKEHVMNPLPQATNPDHKAANKLQGKVALVTGGDSGIGRAVCLCFAKEGATVAFTYVKGHEDRDKDDTLKMLLEAKTSGADNPLAIAADIGFDENCKQVIDLVVKEYGRLDVLVNNAAEQHLTNSVEEITQQQLERVFGTNIFSQFFLVKHALKHMKEGSCIINSTSVNAYNGNPEALDYTATKGAIVAFTRGLSQQLASRGIRVNGVAPGPVWTPIQPASKPAEMIQNLGCEVPMNRVAQPCEIAPCYLFLATCQDSSYFTGQVLHPNGGMVVNA

SEQ ID NO: 486 - protein, 11 aa, Glycine max
EGATVAFTYVK

SEQ ID NO: 487 - protein, 8 aa, Glycine max
QVIDLVVK

SEQ ID NO: 488 - DNA, 879 bp, Glycine max
  source 1..879: mol_type = other DNA
atggcttcgggtgaaaagaaattcccacctcaacaacaacaaacacagcctgggaaggagcatgctatgaatccagtaccccaattcactagccctgactacaagccttcaaataaacttcaaggaaagatagcattagtgactgggggtgactctgggattggacgagcggtgtgtaacttgtttgccttagaaggtgctaccgtgggcttcacatatgtgaaggggcatgaggacaaggacgcgagggacacgttggaaatgatcaagagggcaaagacttcggatgctaaagatccaatggcggtaccagctgatttgggttacgacgagaattgcaagagagtggttgatgaggtcgtgaatgcttatggttgtattgacattctggtcaacaatgcagctgagcaatacgagtgtggaacagtggaggacattgatgagcctaggcttgagagggtctttcgtacaaatatcttctcctatttcttcatgaccaggcatgccttgaagcacatgaaggaaggaagcagcattatcaacacgacatcggtgaatgcatacaagggaaatgcgaaactattggactacacgtccacgaagggagcaattgtggcctatacaaggggacttgctcttcagttggtgagtaagggaattcgggttaatggggtggctcctgggcctatttggaccccattgataccctcctctttcaaggaggaagaaacggctcaatttggtgcccaggtgccaatgaagagagctggccagcctattgaggttgctccgtcttatgtttttcttgcttgcaaccaatgctcctcttacataactggacaagtccttcaccccaatggtggaaccgttgtcaatggt

SEQ ID NO: 489 - protein, 293 aa, Glycine max
MASGEKKFPPQQQQTQPGKEHAMNPVPQFTSPDYKPSNKLQGKIALVTGGDSGIGRAVCNLFALEGATVGFTYVKGHEDKDARDTLEMIKRAKTSDAKDPMAVPADLGYDENCKRVVDEVVNAYGCIDILVNNAAEQYECGTVEDIDEPRLERVFRTNIFSYFFMTRHALKHMKEGSSIINTTSVNAYKGNAKLLDYTSTKGAIVAYTRGLALQLVSKGIRVNGVAPGPIWTPLIPSSFKEEETAQFGAQVPMKRAGQPIEVAPSYVFLACNQCSSYITGQVLHPNGGTVVNG

SEQ ID NO: 490 - protein, 15 aa, Glycine max
EGSSIINTTSVNAYK

SEQ ID NO: 491 - protein, 12 aa, Glycine max
FPPQQQQTQPGK

SEQ ID NO: 492 - protein, 17 aa, Glycine max
GAIVAYTRGLALQLVSK

SEQ ID NO: 493 - protein, 8 aa, Glycine max
LLDYTSTK

SEQ ID NO: 494 - DNA, 3207 bp, Glycine max
  source 1..3207: mol_type = other DNA
atggcttctgagcaaatgcctcgcagagagaacaacagcaatagggtgatagagagagaggttcacgttgagacacacagaaaagagtccctccctgataagggaagggttgtgatagagagtgagaagaaggagaaagagagagagttggcttctgatagagccaggtctggaaatatagttaaggaggacaaagaggttgaagaaggaagaagaggaaaagaaagtggtggtggtggtggtgttcagcatgagaggaagtttgaaacgaaagttgaagagaagaagggttttggggaaaaggctgaactcgagggaataacaagagtggtggtgggaagagagtgcagcaaagaggagaaacaaggaagaacaagagagaaagtagaggcagagaaagcaagatcaatggagaaaactagagatgaagctaaaggaagagaacaagctgaaaccaaaggtcaaaccaagaagggtggtggtgaggaacagggaagaagaagagaagaaataggagaagaaaataaacaatccagcctcgaagagatttctaaatgcagagctgaagctgaagagaaagttaaggagagaaaggagagatcaaaacaagcagcaagtaatgaagctgccaaagagagaaacaagcaggcaaataaggaggcaacaaaggcaaaagatgtcacaagagagaagggtcaacaaggcaatcttaaaacaggaaatgagacacaagacattggccaaaagggttatggtggagcaaaggataccattggtagagtggacaacattactacaccacttgctaaaaaggctcaagttgctggtagcaccactgttcagtatgctggagagaaatcagcacaagccaaagatgctacggtaggatcaatggagaaaactacaaaggatgctaaaggaagagaaaaagcaggaaccaaaggtcaaacaaagaagggtggtggtgaggaacaaggaaaaagaagagaagaaacaggagaagaaaaacaatccagcctggaggagatttctaagcacagagctgaagctcaacaaaaagcaatgagttcaattgaaggtgctaaggagagatacgagagagcaaaacaagaagcaagtaaagcttccaaagagagaaacattcaagcaaataagagggctgaagaagaaagaagaaaagcagaaagtggtaggggtgttcatgtggataagtttgaagagaagaaggattcagtgctgaaagcagaactcgagagaagaacaagagaggtagagggaagagagtggaacaaagaggagaaacaaagaagttgtccaagagggaaagtagaggcagagaaagcaagaccaatggaagaaacaaaactggaagctaaaggaagagaaaaagctgaaaccaaagagggtggtggtgaggaaaaaggaatgagaagagaagaaataggagaagaaaataaacaatccagcctggaagagatttttaagtacagagctgaagctcaacagaaagttaaggagagaaacgagagagcaaaacaagcagcagaagctgccaaagagagaaacaagcaggcaaataaagggaatgaagaaggcagaagagaaagagaaagtggtgggggtgttcatcatgaggctaagtttgaaactaaaggtgaaaaaaagaaggatttagtgggaagagagcggaacaaagaggagaaacaaagaagcagtccaagaggaaaagtagaggcagagaaagcaagaccaatggaagaaacaaaggtggaagctaaaggaagagaaaaagatgtaaccaaagagggtggtgttgaggaacaaggaagaggaagaaaagaaaatgaacaatctagcttgaaagacatttccaagtacagagctgaagctcaacaaaattcattcaatgctactgaagctgctaaggaaagatacgagagggcaaaccaagcgacaagtgaagctgccaaagagagaaacaagcaggcaaataaggaggcaacaaaggcaaaagatgtcacaagagaaatggatcaacaaggcaatcttaaaacagaaaatgagacacaggacaatgaccagaagggttatggtggagcaaagtacaccattggtagtgtggaccacattactacaccatttattgtagaggccaatcttaaatcagaaaatcagatacaagacaatggccaaaagggttatggtggagcaaaggacacaattggtagtgtagaccacattactacaccacttgctgaaaaggccaaagctgctggtagcaccactgctcagtatgttggggagaaagcagcacaagccaaagatgctactgtgcaaactggcaagagtgctgcaaaagttgcctcagatttgagggacaaggctttggccacagggtggagtgctgcacattattctactgagataactgtggagggaaccaaggctgcaacaaatgttgttaagggagttgctgagtatgcaggacagaaggcttctgagcttgcagctatgtcagtggatactgcgaaaggtttagctgctgctgcagctggggagaatgccaaggagtacactgcaaggaagaaggtggaagctgagatggatttggaggccaaaagggaagctcaaaatcaggaatcgaaggagtggccatcagcaaaatccaccactgagagcattcaaagaggacaaggacaacaagaacagcatgagcaaggagaaaaatgggaacatggtgcacaaaggactaataagccacatgaaagcaacattgtggaaacctttgaggaaaacctaggtgggattggggaaaaaagggaacaaaaaccacttggaagcatcattggagagagctttcaaggagttcaagaaaaagggagtgaagtgatggcaagcattggtgaaaaactgggaagtgctgcacatacattagtgaagaagccattggataaggccactgagggagggagagaggtgttgggagctgtaggtgaaactgtggttgagattggtgaaaacatggtgaaaaaaccagcacagaaagtgcagcagcatcaaggtgaaggtggaggaggggtgctgaatgccattggtgaaaccatagcagagatagcagagacaactaaggtgattgttgctggagagggagaaaaggagataatagtaaaaacacatgaatttgaatatggagacttgaaacttgat

SEQ ID NO: 495 - protein, 1069 aa, Glycine max
MASEQMPRRENNSNRVIEREVHVETHRKESLPDKGRVVIESEKKEKERELASDRARSGNIVKEDKEVEEGRRGKESGGGGGVQHERKFETKVEEKKGFGEKAELEGITRVVVGRECSKEEKQGRTREKVEAEKARSMEKTRDEAKGREQAETKGQTKKGGGEEQGRRREEIGEENKQSSLEEISKCRAEAEEKVKERKERSKQAASNEAAKERNKQANKEATKAKDVTREKGQQGNLKTGNETQDIGQKGYGGAKDTIGRVDNITTPLAKKAQVAGSTTVQYAGEKSAQAKDATVGSMEKTTKDAKGREKAGTKGQTKKGGGEEQGKRREETGEEKQSSLEEISKHRAEAQQKAMSSIEGAKERYERAKQEASKASKERNIQANKRAEEERRKAESGRGVHVDKFEEKKDSVLKAELERRTREVEGREWNKEEKQRSCPRGKVEAEKARPMEETKLEAKGREKAETKEGGGEEKGMRREEIGEENKQSSLEEIFKYRAEAQQKVKERNERAKQAAEAAKERNKQANKGNEEGRRERESGGGVHHEAKFETKGEKKKDLVGRERNKEEKQRSSPRGKVEAEKARPMEETKVEAKGREKDVTKEGGVEEQGRGRKENEQSSLKDISKYRAEAQQNSFNATEAAKERYERANQATSEAAKERNKQANKEATKAKDVTREMDQQGNLKTENETQDNDQKGYGGAKYTIGSVDHITTPFIVEANLKSENQIQDNGQKGYGGAKDTIGSVDHITTPLAEKAKAAGSTTAQYVGEKAAQAKDATVQTGKSAAKVASDLRDKALATGWSAAHYSTEITVEGTKAATNVVKGVAEYAGQKASELAAMSVDTAKGLAAAAAGENAKEYTARKKVEAEMDLEAKREAQNQESKEWPSAKSTTESIQRGQGQQEQHEQGEKWEHGAQRTNKPHESNIVETFEENLGGIGEKREQKPLGSIIGESFQGVQEKGSEVMASIGEKLGSAAHTLVKKPLDKATEGGREVLGAVGETVVEIGENMVKKPAQKVQQHQGEGGGGVLNAIGETIAEIAETTKVIVAGEGEKEIIVKTHEFEYGDLKLD

SEQ ID NO: 496 - protein, 13 aa, Glycine max
AAGSTTAQYVGEK

SEQ ID NO: 497 - protein, 21 aa, Glycine max
ALATGWSAAHYSTEITVEGTK

SEQ ID NO: 498 - protein, 15 aa, Glycine max
AQVAGSTTVQYAGEK

SEQ ID NO: 499 - protein, 15 aa, Glycine max
DTIGRVDNITTPLAK

SEQ ID NO: 500 - protein, 16 aa, Glycine max
DTIGSVDHITTPLAEK

SEQ ID NO: 501 - protein, 16 aa, Glycine max
ERELASDRARSGNIVK

SEQ ID NO: 502 - protein, 15 aa, Glycine max
ERYERANQATSEAAK

SEQ ID NO: 503 - protein, 18 aa, Glycine max
GGGEEQGRRREEIGEENK

SEQ ID NO: 504 - protein, 12 aa, Glycine max
GLAAAAAGENAK

SEQ ID NO: 505 - protein, 9 aa, Glycine max
GRVVIESEK

SEQ ID NO: 506 - protein, 9 aa, Glycine max
GVAEYAGQK

SEQ ID NO: 507 - protein, 10 aa, Glycine max
LGSAAHTLVK

SEQ ID NO: 508 - protein, 9 aa, Glycine max
QSSLEEIFK

SEQ ID NO: 509 - protein, 9 aa, Glycine max
QSSLEEISK

SEQ ID NO: 510 - protein, 20 aa, Glycine max
REQKPLGSIIGESFQGVQEK

SEQ ID NO: 511 - protein, 11 aa, Glycine max
TGNETQDIGQK

SEQ ID NO: 512 - protein, 9 aa, Glycine max
VIVAGEGEK

SEQ ID NO: 513 - protein, 28 aa, Glycine max
VQQHQGEGGGGVLNAIGETIAEIAETTK

SEQ ID NO: 514 - DNA, 735 bp, Glycine max
  source 1..735: mol_type = other DNA
atggcactgtgcaaccgcgtcagatcgggaatctctctcttcaccaaatccacgttcctcacttcccagcgatccactctccaacgatccctcgttgcccctttcatttccctcgcctccagaaattacgccaacgtgccggggcaaaaggaaaccaaagttaaggtccctctggctttgtttggaggttcaggaaactatgcctctgctttgtatattgcatctgtgaaagccaatgcagtggaaaaggttgagactgagcttctgcaatttgttgaggcagtaaagaatagtcccatattttcacagtttataagggacttgtctgtggctaaggatgttagagttaaagttatccaggatatttgtggggaagccaagttttcggatgttacaaagaacttccttgttattgttgctgagaatggaaggcttaaaaacatagacaccattgcaaagaggtttgcacagttgtcaatggcatataagggagaagtgcaagcgactgtgaccactgttgttcctcttccccctgaagaggaaaaggcattgaaggagactcttcaggaaataataggttctggagcgaaggttcatcttgaacagaagattgatccaagcatacttggtgggcttgtgcttgaatttagccagaaggtctttgacatgtcaattaagaccagggcacaacagatggagaggatcctccgagagcccgtgtccattgccaacatc

SEQ ID NO: 515 - protein, 245 aa, Glycine max
MALCNRVRSGISLFTKSTFLTSQRSTLQRSLVAPFISLASRNYANVPGQKETKVKVPLALFGGSGNYASALYIASVKANAVEKVETELLQFVEAVKNSPIFSQFIRDLSVAKDVRVKVIQDICGEAKFSDVTKNFLVIVAENGRLKNIDTIAKRFAQLSMAYKGEVQATVTTVVPLPPEEEKALKETLQEIIGSGAKVHLEQKIDPSILGGLVLEFSQKVFDMSIKTRAQQMERILREPVSIANI

SEQ ID NO: 516 - protein, 12 aa, Glycine max
ETLQEIIGSGAK

SEQ ID NO: 517 - DNA, 678 bp, Glycine max
  source 1..678: mol_type = other DNA
atgaccacacaagtaccaccacacagtgtccaagtccacacaacaacacaccgctacgaagctggcgtcgtcccccccggagcccgtttcgaaccgccgcgttatgaagccggcgtcaaagcgccctccatttaccattccgagagaggtccaacgacttcccaggtcctcgccgtcctcgccggcctcccggtcggtggcatcctgctcctcctggcgggattgaccctggcaggaaccctcaccggactggcagttgccacgccgctcttcgtcctcttcagcccggtgctggttccagccacggtcgccatcggcctggccgtggccgggttcctgacgtcgggggccttcgggctcacggcgctgtcttccttctcctggatcctgaactacatccgggagacccagccagcctcggagaacctcgccgccgccgcgaagcatcacctcgccgaggcggcggagtacgtggggcagaagacgaaggaagtggggcagaagaccaaggaggttgggcaggatattcagagcaaggcacaggatacaagggaggcagctgcaagggatgcaagggaggcagctgcaagggatgcaagggaagctgctgcaagggatgcaaaggtggaggcaagggatgtaaagagaacaacagtgacagcaacaaccgcaaccgca

SEQ ID NO: 518 - protein, 226 aa, Glycine max
MTTQVPPHSVQVHTTTHRYEAGVVPPGARFEPPRYEAGVKAPSIYHSERGPTTSQVLAVLAGLPVGGILLLLAGLTLAGTLTGLAVATPLFVLFSPVLVPATVAIGLAVAGFLTSGAFGLTALSSFSWILNYIRETQPASENLAAAAKHHLAEAAEYVGQKTKEVGQKTKEVGQDIQSKAQDTREAAARDAREAAARDAREAAARDAKVEARDVKRTTVTATTATA

SEQ ID NO: 519 - protein, 9 aa, Glycine max
EVGQDIQSK

SEQ ID NO: 520 - protein, 11 aa, Glycine max
RTTVTATTATA

SEQ ID NO: 521 - DNA, 627 bp, Glycine max
  source 1..627: mol_type = other DNA
atgacaaggaaacaagcaagttccacgagacgtggcggtctttcaggagtcttgcattccaaatccaaatcttctcctctactctccatatcccttgtcctatttgtagccatccttctcatcctttatgcttgtatcgggtcaggtatattgggaggaagaaaggatgtgattagcatggctgaagatgatttttcatgcacttttgaagttccaagtgcaattcctgttttgaagaatgcatatggtggcagcatgaaaaatgttttgcacgtaggccctgagagttgttcggtggtctccaaatttttaagagaaggggaaactgaagcatggggtgtggagccatatgacttagacgatgctgataggaactgcaaggctcttgtgcagaaaggcattatacgtgttgcagatataaaattccctttaccatacagggtgaagtctttttctcatgttatagtgtcagatgctttggattacctgtctccaaaatatataaacaaaactcttcctgagctagcaagggtatcttctgatggtattattattttcacaggttacccaggtcaaccgagagctaaaatagcaccattatccaaatttggccgtccacataggtgc

SEQ ID NO: 522 - protein, 209 aa, Glycine max
MTRKQASSTRRGGLSGVLHSKSKSSPLLSISLVLFVAILLILYACIGSGILGGRKDVISMAEDDFSCTFEVPSAIPVLKNAYGGSMKNVLHVGPESCSVVSKFLREGETEAWGVEPYDLDDADRNCKALVQKGIIRVADIKFPLPYRVKSFSHVIVSDALDYLSPKYINKTLPELARVSSDGIIIFTGYPGQPRAKIAPLSKFGRPHRC

SEQ ID NO: 523 - protein, 8 aa, Glycine max
NAYGGSMK

SEQ ID NO: 524 - DNA, 723 bp, Glycine max
  source 1..723: mol_type = other DNA
atggccgcgcgagctctgttgaccagaaaaaccctagccaccgtgctccgcaacgacgcgaagcccataatcggagttggcataacagcagcggctactcattcacgcgggttgcacgtgtacacgctacccgatctggattacgactatggcgcactggagccagccatcagcggcgacatcatgcagctgcaccaccagaagcaccaccagacttacatcaccaactacaacaaggccctcgagcagctccaagacgccatcgccaagaaagattcctccgccgtcgttaagctccagggcgccatcaagttcaacggcggaggtcatgtcaaccattctattttctggaaaaatctagctcctgttcgtgaaggaggtggtgaaccacccaagggttcactgggatgggctattgacacacattttggttcttttgaagcattaatacaaaaagttaacgcagaaggtgctgcactacaggggtctggatgggtgtggcttggtctggacaaagagttgaagaggcttgtagttgaaaccactgccaaccaggacccactggttactaagggaccaaatttggttccattgattggtattgatgtttgggagcatgcgtactacttacagtacaagaatgttagaccagactatctgaagaacatttggaaagttattaattggaaatatgccagtgaagtgtatgagaaagagagctct

SEQ ID NO: 525 - protein, 241 aa, Glycine max
MAARALLTRKTLATVLRNDAKPIIGVGITAAATHSRGLHVYTLPDLDYDYGALEPAISGDIMQLHHQKHHQTYITNYNKALEQLQDAIAKKDSSAVVKLQGAIKFNGGGHVNHSIFWKNLAPVREGGGEPPKGSLGWAIDTHFGSFEALIQKVNAEGAALQGSGWVWLGLDKELKRLVVETTANQDPLVTKGPNLVPLIGIDVWEHAYYLQYKNVRPDYLKNIWKVINWKYASEVYEKESS

SEQ ID NO: 526 - protein, 11 aa, Glycine max
ALEQLQDAIAK

SEQ ID NO: 527 - protein, 11 aa, Glycine max
HHQTYITNYNK

SEQ ID NO: 528 - protein, 14 aa, Glycine max
NLAPVREGGGEPPK

SEQ ID NO: 529 - protein, 8 aa, Glycine max
NVRPDYLK

SEQ ID NO: 530 - protein, 16 aa, Glycine max
RLVVETTANQDPLVTK

SEQ ID NO: 531 - protein, 8 aa, Glycine max
YASEVYEK

SEQ ID NO: 532 - DNA, 720 bp, Glycine max
  source 1..720: mol_type = other DNA
atggccgcgcgagccctgttgaccagaaaaaccctagccaccgttctccgcaacgacgcgaagcccataatcggagctggcatagctgcagctactcattctcgtgggttgcacgtgtacacgctccccgatctggattacgactatggcgctctggagccagccatcagcggcgaaatcatgcagctgcaccaccagaagcaccaccagacttacatcaccaacttcaacaaggccctcgagcagctccaagacgccgtcgccaagaaagattcctccgccgtcgttaagctccagggcgccatcaagttcaacggcggaggtcacgtcaaccattctattttctggaaaaatctagctcctgttcgtgaaggaggtggtgaaccacccaagggttcactgggatgggccattgacacacattttggctcttttgaagcattagtacaaaaagttaatgcagaaggtgctgcactacaggggtctggatgggtgtggcttggtctggacaaagagttgaagaggcttgtagttgaaaccactgccaaccaggacccactggttactaagggaccaaatttggttccattgcttggtattgatgtttgggagcatgcgtactacttacagtacaagaatgttagaccagactatctgaagaacatttggaaagttattaattggaaatatgccagtgaagtgtatgagaaagagagctct

SEQ ID NO: 533 - protein, 240 aa, Glycine max
MAARALLTRKTLATVLRNDAKPIIGAGIAAATHSRGLHVYTLPDLDYDYGALEPAISGEIMQLHHQKHHQTYITNFNKALEQLQDAVAKKDSSAVVKLQGAIKFNGGGHVNHSIFWKNLAPVREGGGEPPKGSLGWAIDTHFGSFEALVQKVNAEGAALQGSGWVWLGLDKELKRLVVETTANQDPLVTKGPNLVPLLGIDVWEHAYYLQYKNVRPDYLKNIWKVINWKYASEVYEKESS

SEQ ID NO: 534 - protein, 11 aa, Glycine max
ALEQLQDAVAK

SEQ ID NO: 535 - protein, 11 aa, Glycine max
HHQTYITNFNK

SEQ ID NO: 536 - protein, 14 aa, Glycine max
NLAPVREGGGEPPK

SEQ ID NO: 537 - protein, 8 aa, Glycine max
NVRPDYLK

SEQ ID NO: 538 - protein, 16 aa, Glycine max
RLVVETTANQDPLVTK

SEQ ID NO: 539 - protein, 8 aa, Glycine max
YASEVYEK

SEQ ID NO: 540 - DNA, 486 bp, Glycine max
  source 1..486: mol_type = other DNA
atggctccaattgcagtgggagatgtcattccagacggcattttggcttacttggatgaagaaaacaaaccccaaactgtctctattcactctctcgccgccggcaagaaagtcatcatctttggggttcccggtgccttcactcctacatgcagcttgaagcacgtgcctggcttcattgagagagcagaagagctgaaaggaaagggtgtggacgaaataatctgtatcagcgtgaatgatccctttgtgatgaactcatgggccaaaacgttcccagagaacaagcatgttaagttccttgctgatggtgcagccaaatacaccaatgccctcggtcttgagcttgacctcaccgacaagggtctcggcgtccgctcaaagaggtttgctttgctggtggaagacctcaaggtgaaggttgcaaatgttgaaagtggaggagagttcaccatctccagtgctgaagagatcatcaaggccctc

SEQ ID NO: 541 - protein, 162 aa, Glycine max
MAPIAVGDVIPDGILAYLDEENKPQTVSIHSLAAGKKVIIFGVPGAFTPTCSLKHVPGFIERAEELKGKGVDEIICISVNDPFVMNSWAKTFPENKHVKFLADGAAKYTNALGLELDLTDKGLGVRSKRFALLVEDLKVKVANVESGGEFTISSAEEIIKAL

SEQ ID NO: 542 - protein, 13 aa, Glycine max
HVPGFIERAEELK

SEQ ID NO: 543 - protein, 10 aa, Glycine max
RFALLVEDLK

SEQ ID NO: 544 - protein, 20 aa, Glycine max
VANVESGGEFTISSAEEIIK

SEQ ID NO: 545 - protein, 14 aa, Glycine max
YTNALGLELDLTDK

SEQ ID NO: 546 - DNA, 486 bp, Glycine max
  source 1..486: mol_type = other DNA
atggctccaattgcagtgggaaacgtgattccagacggcattttggcttacttggatgaagaaaacaaaccccaaactgtctccattcactctctcgccaccggcaagaaagtcatcatctttggggttcccggtgccttcactcctacttgcagcttgaagcacgttcctggcttcattgagagagcagaagagctgaaagggaagggtgtggacgaaataatctgtatcagtgtgaatgatccatttgtgatgaactcatgggccaaaacattcccagagaacaagcatgttaagttccttgctgatggtgcagccaaatacaccaatgcccttggtcttgagcttgacctcactgacaagggtctcggcgtccgctcaaagaggtttgctctgctggtggaagacctcaaggtgaaggttgcgaatgttgaaagtggaggagagttcaccatctccagtgctgaagagatcatcaaggccctt

SEQ ID NO: 547 - protein, 162 aa, Glycine max
MAPIAVGNVIPDGILAYLDEENKPQTVSIHSLATGKKVIIFGVPGAFTPTCSLKHVPGFIERAEELKGKGVDEIICISVNDPFVMNSWAKTFPENKHVKFLADGAAKYTNALGLELDLTDKGLGVRSKRFALLVEDLKVKVANVESGGEFTISSAEEIIKAL

SEQ ID NO: 548 - protein, 13 aa, Glycine max
HVPGFIERAEELK

SEQ ID NO: 549 - protein, 10 aa, Glycine max
RFALLVEDLK

SEQ ID NO: 550 - protein, 20 aa, Glycine max
VANVESGGEFTISSAEEIIK

SEQ ID NO: 551 - protein, 14 aa, Glycine max
YTNALGLELDLTDK

SEQ ID NO: 552 - DNA, 456 bp, Glycine max
  source 1..456: mol_type = other DNA
atgtcgcagttgctggataaagccaagaacttcgtgtcggagaaggtaaatgatatggccaagccggaggcgagtgtcactgacgtggacttcaagcgcgtgagcaaagataatgtcgagtacttggccaaggtctctgttcgtaacccttattcaacttccatacccatttgtgagatcaactactctttcaaaagcgcatccagggagatagcatcagggaaaattccagacccaggatcgttgaaggcaaaggacacaacaatggtggatgtgccagttaaggttccatacagcatattgatgagcttggcaaaggacattggtgctgactgggacatagactatcaattggatcttggtctggttattgatgttcctgtcattggcatcttcaccattcctctctctcagaaaggagagatcaagctaccaaccctctctaccatgttcgcc

SEQ ID NO: 553 - protein, 152 aa, Glycine max
MSQLLDKAKNFVSEKVNDMAKPEASVTDVDFKRVSKDNVEYLAKVSVRNPYSTSIPICEINYSFKSASREIASGKIPDPGSLKAKDTTMVDVPVKVPYSILMSLAKDIGADWDIDYQLDLGLVIDVPVIGIFTIPLSQKGEIKLPTLSTMFA

SEQ ID NO: 554 - protein, 8 aa, Glycine max
DNVEYLAK

SEQ ID NO: 555 - DNA, 480 bp, Glycine max
  source 1..480: mol_type = other DNA
atgtcggacgaagagcaccatttcgagtccaaggcagacgccggtgcctccaaaacctaccctcagcaagccggtaccattcgcaagaacggctacatcgtcatcaaaggacgcccctgcaaggttgtggaagtttcgacttccaaaactggaaagcacggtcacgccaagtgccactttgttggaattgatattttcactgccaagaaacttgaggatattgtgccctcttctcacaactgtgatgttcctcatgtgaatcgtactgattaccagctcattgatattgctgaggatggatttttgagtctgcttactgaaaatgggaacaccaaggatgacctcaagcttcccactgatgagagtctgctcactcagataaaggatggatttgctgagggcaaggatcttgttgtgtctgtcatgtctgctatgggtgaggaacagatttgcgccctgaaggatattgggccaaagaac

SEQ ID NO: 556 - protein, 160 aa, Glycine max
MSDEEHHFESKADAGASKTYPQQAGTIRKNGYIVIKGRPCKVVEVSTSKTGKHGHAKCHFVGIDIFTAKKLEDIVPSSHNCDVPHVNRTDYQLIDIAEDGFLSLLTENGNTKDDLKLPTDESLLTQIKDGFAEGKDLVVSVMSAMGEEQICALKDIGPKN

SEQ ID NO: 557 - protein, 12 aa, Glycine max
LPTDESLLTQIK

SEQ ID NO: 558 - protein, 7 aa, Glycine max
NGYIVIK

SEQ ID NO: 559 - DNA, 480 bp, Glycine max
  source 1..480: mol_type = other DNA
atgtcggacgaagagcatcacttcgattctcaggcggatgcgggagcgtccaagacgtatcctcagcaggctggtaccattcgcaagaatggctacatcgtcatcaaatccaggccttgcaaggttgtagaggtctctacgtcaaagacaggcaaacatggacatgcaaagtgtcactttgctgcaattgatatttttaatgggaaaaagcttgaagatattgttccatcatcccacaactgtgatgttccacatgtgaatcgcactgactatcagcttattgatatctctgaagacggctttgtgagtctgttgactgataatggaaataccaaggatgatctgaagcttcctactgatgaggcattgcttgctcagattaaagatggattttctgaaggaaaagaccttgtggtttctgttatgtctgcaatgggagaagaacagatctgttcactgaaggacatcggtcccaaaaat

SEQ ID NO: 560 - protein, 160 aa, Glycine max
MSDEEHHFDSQADAGASKTYPQQAGTIRKNGYIVIKSRPCKVVEVSTSKTGKHGHAKCHFAAIDIFNGKKLEDIVPSSHNCDVPHVNRTDYQLIDISEDGFVSLLTDNGNTKDDLKLPTDEALLAQIKDGFSEGKDLVVSVMSAMGEEQICSLKDIGPKN

SEQ ID NO: 561 - protein, 7 aa, Glycine max
NGYIVIK

SEQ ID NO: 562 - protein, 11 aa, Glycine max
TYPQQAGTIRK

SEQ ID NO: 563 - protein, 8 aa, Glycine max
VVEVSTSK

SEQ ID NO: 564 - DNA, 501 bp, Glycine max
  source 1..501: mol_type = other DNA
atggctagccaatcaaacactaaatcagttcacgatttcacagttaaagatgccaggggaaataatgttaatctcgccgattacaaaggaaaggtccttctcattgtcaatgttgcctcacaatgcggcttgactaattcaaactacacagagcttaaccaattgtatgagaaatacaaagggaaaggacttgaaattctggcattcccatgcaatcagtttggggctcaggagccaggaactaatgaagagattgtggagtttgcttgtactcgcttcaaagctgagtttcccattttcgacaaggtggatgtgaatggtgacaatgctgctccactgtacaagtttctgaaatcaagcaaaggtggactctttggggatagtatcaaatggaacttctccaaattccttgttgataaggatgggaatgtcgttgaccgttatgcacccacaacttctcctctcagcattgagaaagacataaagaagctgctagatgca

SEQ ID NO: 565 - protein, 167 aa, Glycine max
MASQSNTKSVHDFTVKDARGNNVNLADYKGKVLLIVNVASQCGLTNSNYTELNQLYEKYKGKGLEILAFPCNQFGAQEPGTNEEIVEFACTRFKAEFPIFDKVDVNGDNAAPLYKFLKSSKGGLFGDSIKWNFSKFLVDKDGNVVDRYAPTTSPLSIEKDIKKLLDA

SEQ ID NO: 566 - protein, 8 aa, Glycine max
AEFPIFDK

SEQ ID NO: 567 - protein, 13 aa, Glycine max
DARGNNVNLADYK

SEQ ID NO: 568 - protein, 19 aa, Glycine max
DGNVVDRYAPTTSPLSIEK

SEQ ID NO: 569 - protein, 9 aa, Glycine max
GGLFGDSIK

SEQ ID NO: 570 - protein, 13 aa, Glycine max
VDVNGDNAAPLYK

SEQ ID NO: 571 - DNA, 357 bp, Glycine max
  source 1..357: mol_type = other DNA
atggcttccgaagaatcccagaacaagccccccagcgagcagtccactaccgaactcatcacgagcgcgaagctggtggcggaggcggcgcagtcggctctgaagagcgacagcgacaaagtggacaaggccaaggtggctgacgctgccggcgatcttctcgacgcggcggggaagtatggaaaacttgatgacaaacaaggcatagggcaatatgttgacaaggctgctgattatctgcataattaccagggtgataataccactgctccttcacagcctgcagagtctaaaggtgaagaaagtggtggtgggttaggtggtcttgcaaacttggctggaggcttcttcaaaaag

SEQ ID NO: 572 - protein, 119 aa, Glycine max
MASEESQNKPPSEQSTTELITSAKLVAEAAQSALKSDSDKVDKAKVADAAGDLLDAAGKYGKLDDKQGIGQYVDKAADYLHNYQGDNTTAPSQPAESKGEESGGGLGGLANLAGGFFKK

SEQ ID NO: 573 - protein, 23 aa, Glycine max
AADYLHNYQGDNTTAPSQPAESK

SEQ ID NO: 574 - protein, 11 aa, Glycine max
LVAEAAQSALK

SEQ ID NO: 575 - protein, 9 aa, Glycine max
QGIGQYVDK

SEQ ID NO: 576 - protein, 14 aa, Glycine max
VADAAGDLLDAAGK

SEQ ID NO: 577 - DNA, 777 bp, Glycine max
  source 1..777: mol_type = other DNA
atggcggccgctccctcgcctcgcgaagagaacgtctacatggcgaagctcgccgagcaggccgagcgctacgaagagatggtggagttcatggagaaggtctccgccgccgccgacaacgaggaacttaccgtggaggagaggaacctcctctccgtcgcctacaagaacgtgatcggagcgcgccgcgcctcgtggcgcatcatctcctcgatcgagcagaaggaggagagccgcggcaacgaggatcacgtctccgtcatccgcgactaccgatccaagatcgagtccgagctctccaacatctgcgacggtatcctcaagctcctcgactctcgcctcattccatccgcttcctccggcgattccaaggtcttctacctcaagatgaagggagattaccacagataccttgccgagttcaagaccggcgccgagcgtaaggaggctgccgagagcactctctctgcatacaaagccgcacaggacattgcgaatgccgaactgcctcctactcatccaattaggcttggccttgctcttaacttttctgtattttactacgaaatccttaattctccggatcgtgcctgcaaccttgcaaaacaggcttttgatgaagctattgctgaattggatacactgggagaggagtcatacaaggatagcactttgatcatgcaactccttcgtgataacctcacactttggacctctgacatgcaggacgatggagcagatgaaattaaagaagcagcaccgaaaccggatgaccag

SEQ ID NO: 578 - protein, 259 aa, Glycine max
MAAAPSPREENVYMAKLAEQAERYEEMVEFMEKVSAAADNEELTVEERNLLSVAYKNVIGARRASWRIISSIEQKEESRGNEDHVSVIRDYRSKIESELSNICDGILKLLDSRLIPSASSGDSKVFYLKMKGDYHRYLAEFKTGAERKEAAESTLSAYKAAQDIANAELPPTHPIRLGLALNFSVFYYEILNSPDRACNLAKQAFDEAIAELDTLGEESYKDSTLIMQLLRDNLTLWTSDMQDDGADEIKEAAPKPDDQ

SEQ ID NO: 579 - protein, 11 aa, Glycine max
EAAESTLSAYK

SEQ ID NO: 580 - protein, 16 aa, Glycine max
LLDSRLIPSASSGDSK

SEQ ID NO: 581 - protein, 19 aa, Glycine max
QAFDEAIAELDTLGEESYK

SEQ ID NO: 582 - protein, 23 aa, Glycine max
VSAAADNEELTVEERNLLSVAYK

SEQ ID NO: 583 - DNA, 993 bp, Glycine max
  source 1..993: mol_type = other DNA
atggcgttgaacaatggcgtccccgccaccggcaccggcctcatcgtcagcttcggtgagatgctcatcgacttcgtccccaccgtctctggcgtgtccctggccgaggcccctggcttcctcaaggcccccggcggcgcccccgctaacgtcgccatcgccgtgtcgcgcctcggcggcaaagccgccttcgtcggcaagctcggcgacgacgagttcggccacatgctcgccggaatcctcaaggaaaacggcgttcgcgccgacggcatcaactttgaccagggcgcacgcaccgccctggccttcgtgaccctacgcgccgacggggagcgtgagttcatgttctacagaaaccccagcgccgacatgctcctcaagcccgaagaactcaatctcgaactcatcagatctgcaaaagttttccattacggatcaatcagtttgatcgtggagccatgcagatcagcacacttgaaggcaatggaagtagccaaggaatctgggtgcttgctctcctatgaccccaaccttcgtctacctttgtggccttcggctgaggaagctcgtaagcaaatactgagcatttgggagaaggctgatttgatcaaggtcagtgatgcggagcttgagttcctcacaggaagtgacaagattgatgatgaatctgctttgtcattgtggcaccccaatttgaagttgctccttgtcactcttggggaacatggttccagatactacaccaagagtttcaaaggatcggtagatgctttccatgtcaatacagttgatacaactggtgccggtgattcctttgttggtgctttattggccaagattgtcgatgatcagtccatacttgaagatgaaccaaggttaagagaagtactaaagtttgcaaatgcatgtggagctattacaactacccaaaagggagcaattccggcccttcccaaagaggaggctgcactgaaactgatcaaaggggggtca

SEQ ID NO: 584 - protein, 331 aa, Glycine max
MALNNGVPATGTGLIVSFGEMLIDFVPTVSGVSLAEAPGFLKAPGGAPANVAIAVSRLGGKAAFVGKLGDDEFGHMLAGILKENGVRADGINFDQGARTALAFVTLRADGEREFMFYRNPSADMLLKPEELNLELIRSAKVFHYGSISLIVEPCRSAHLKAMEVAKESGCLLSYDPNLRLPLWPSAEEARKQILSIWEKADLIKVSDAELEFLTGSDKIDDESALSLWHPNLKLLLVTLGEHGSRYYTKSFKGSVDAFHVNTVDTTGAGDSFVGALLAKIVDDQSILEDEPRLREVLKFANACGAITTTQKGAIPALPKEEAALKLIKGGS

SEQ ID NO: 585 - protein, 19 aa, Glycine max
APGGAPANVAIAVSRLGGK

SEQ ID NO: 586 - protein, 27 aa, Glycine max
GSVDAFHVNTVDTTGAGDSFVGALLAK

SEQ ID NO: 587 - protein, 19 aa, Glycine max
IVDDQSILEDEPRLREVLK

SEQ ID NO: 588 - protein, 14 aa, Glycine max
VSDAELEFLTGSDK

SEQ ID NO: 589 - DNA, 759 bp, Glycine max
  source 1..759: mol_type = other DNA
atgggcagaaagttcttcgttggtggcaactggaaatgcaatgggaccactgaggaggtgaagaagattgttactactttaaatgaagctaaagtccctggagaagatgttgtagaagttgttgtgagccctccttttgtgttccttccttttgtaaaaagtttgctgcgccctgatttccatgtctcggcccaaaattgttgggttcgcaaaggtggtgcttatactggagaggttagtgctgaaatgcttgttaatttgggaattccttgggttattattggtcactctgaacggaggcagcttttgaatgaatcaaatgagtttgtgggagataaagttgcctatgcacttcaacaaggtctgaaagttatagcatgcattggggaaactcttgaacagcgtgaagctggtacaacaacggctgttgttgctgagcaaacaaaagcaattgcagctaaaatatcaaattgggacaatgtcgttttggcctatgagccagtttgggccattggaacaggaaaggttgcaactcctgctcaggctcaagaggttcatgctgatttaaggaaatgggttcatgacaatgtgagtgctgaagttgctgcatctgtaagaattatctatggaggctctgtaaatggaggaaactgcaaagaattggcagcacagcccgatgttgatggatttttggttggtggtgcatccctcaaggcggaatttgtggacatcataaacgctgccactgtgaagaagaat

SEQ ID NO: 590 - protein, 253 aa, Glycine max
MGRKFFVGGNWKCNGTTEEVKKIVTTLNEAKVPGEDVVEVVVSPPFVFLPFVKSLLRPDFHVSAQNCWVRKGGAYTGEVSAEMLVNLGIPWVIIGHSERRQLLNESNEFVGDKVAYALQQGLKVIACIGETLEQREAGTTTAVVAEQTKAIAAKISNWDNVVLAYEPVWAIGTGKVATPAQAQEVHADLRKWVHDNVSAEVAASVRIIYGGSVNGGNCKELAAQPDVDGFLVGGASLKAEFVDIINAATVKKN

SEQ ID NO: 591 - protein, 13 aa, Glycine max
AEFVDIINAATVK

SEQ ID NO: 592 - protein, 19 aa, Glycine max
ELAAQPDVDGFLVGGASLK

SEQ ID NO: 593 - protein, 8 aa, Glycine max
FFVGGNWK

SEQ ID NO: 594 - protein, 9 aa, Glycine max
IVTTLNEAK

SEQ ID NO: 595 - protein, 16 aa, Glycine max
VATPAQAQEVHADLRK

SEQ ID NO: 596 - protein, 10 aa, Glycine max
VAYALQQGLK

SEQ ID NO: 597 - protein, 22 aa, Glycine max
VPGEDVVEVVVSPPFVFLPFVK

SEQ ID NO: 598 - DNA, 759 bp, Glycine max
  source 1..759: mol_type = other DNA
atgggcagaaaattcttcgttggtggcaactggaaatgcaatgggaccactgaggaggtaaagaagattgttactactttgaatgaggctaaagtccctggagaagatgtcgtagaagttgttgtgagccctccttttgtgttccttcctgttgtaaaaagtttgctgcgccctgatttccatgtttcggcacaaaactgttgggttcgcaaaggtggtgcttataccggtgaggttagtgctgaaatgcttgttaatttgggaattccttgggttattattggtcactctgaacggaggcagcttttaaatgaatcaaatgagtttgtgggagataaagttgcctatgcacttcaacaaggtctaaaagttattgcatgcattggggagactctcgaacagcgtgaagctggtacaacaacggctgttgtttctgagcaaacaaaagcaattgcagctaaaatatcaaattgggacaatgtcgttttggcctacgagccagtttgggccattggaacaggaaaggttgctactcctgctcaggctcaagaggtccatgctgatttgaggaaatgggttcatgacaatgtgagtgctgaagttgctgcatctgtaagaattatctatggaggttctgtaaatggaggaaactgcaaagaattggccgcacagcccgatgttgatggatttttggttggtggtgcctccctgaagccggagttcgtggacatcataaatgctgccactgtgaagaagaat

SEQ ID NO: 599 - protein, 253 aa, Glycine max
MGRKFFVGGNWKCNGTTEEVKKIVTTLNEAKVPGEDVVEVVVSPPFVFLPVVKSLLRPDFHVSAQNCWVRKGGAYTGEVSAEMLVNLGIPWVIIGHSERRQLLNESNEFVGDKVAYALQQGLKVIACIGETLEQREAGTTTAVVSEQTKAIAAKISNWDNVVLAYEPVWAIGTGKVATPAQAQEVHADLRKWVHDNVSAEVAASVRIIYGGSVNGGNCKELAAQPDVDGFLVGGASLKPEFVDIINAATVKKN

SEQ ID NO: 600 - protein, 32 aa, Glycine max
ELAAQPDVDGFLVGGASLKPEFVDIINAATVK

SEQ ID NO: 601 - protein, 8 aa, Glycine max
FFVGGNWK

SEQ ID NO: 602 - protein, 9 aa, Glycine max
IVTTLNEAK

SEQ ID NO: 603 - protein, 16 aa, Glycine max
VATPAQAQEVHADLRK

SEQ ID NO: 604 - protein, 10 aa, Glycine max
VAYALQQGLK

SEQ ID NO: 605 - DNA, 765 bp, Glycine max
  source 1..765: mol_type = other DNA
atggcctcattgggtgggttacaaaatgtgagcggcatcaattttcttatcaaagagaaaggatgttgtatgctgcagggtccaaaagtcaatgcaaagttcgagctgaagccgccaccatatccactgaatggtttggagccggtgatgagccagcagacacttgagtttcactgggggaagcaccacaagacttatgtggaaaatctgaaaaaacaagttgttgggacagagcttgatgggaagtcactagaagagattattgtcacatcatacaataagggtgacattcttccagctttcaacaatgcagcacaggtatggaaccatgacttcttctgggagtgcatgaaaccaggtggaggtggaaagccatccggggagcttctagaactgattgaaagagactttggttcatttgtaaaattccttgatgagttcaaggctgctgctgcaacacaatttggttcagggtgggcttggctagcatatagagcaagaaaatttgatggggaaaatgtagcaaatcctccttcacccgatgaggacaacaagctagtggtgctcaagagtcccaatgctgtgaacccccttgtttggggaggttactacccacttcttaccattgatgtttgggagcatgcttactaccttgattttcagaaccggcgtcctgattatatatcagtgttcatggataagcttgtttcctgggatgcagtgagctctagacttgaacaagctaaggctttaattaccagtgca

SEQ ID NO: 606 - protein, 255 aa, Glycine max
MASLGGLQNVSGINFLIKEKGCCMLQGPKVNAKFELKPPPYPLNGLEPVMSQQTLEFHWGKHHKTYVENLKKQVVGTELDGKSLEEIIVTSYNKGDILPAFNNAAQVWNHDFFWECMKPGGGGKPSGELLELIERDFGSFVKFLDEFKAAAATQFGSGWAWLAYRARKFDGENVANPPSPDEDNKLVVLKSPNAVNPLVWGGYYPLLTIDVWEHAYYLDFQNRRPDYISVFMDKLVSWDAVSSRLEQAKALITSA

SEQ ID NO: 607 - protein, 17 aa, Glycine max
FDGENVANPPSPDEDNK

SEQ ID NO: 608 - protein, 15 aa, Glycine max
LVSWDAVSSRLEQAK

SEQ ID NO: 609 - protein, 10 aa, Glycine max
QVVGTELDGK

SEQ ID NO: 610 - protein, 12 aa, Glycine max
SLEEIIVTSYNK

SEQ ID NO: 611 - protein, 7 aa, Glycine max
TYVENLK

SEQ ID NO: 612 - DNA, 732 bp, Glycine max
  source 1..732: mol_type = other DNA
atggcctcattgggtgggttacaaaatctaagcggcatcaattttcttatcaaaaaagagggtccaaaagtcaatgcgaagttcgagctgaagccgccaccatatccactgaatggtttggagccggtgatgagccagcagacacttgagtttcattgggggaaacaccacaagacatatgtggaaaatctgaaaaaacaaattgttgggacagagcttgatgggaaatcactagaagagattatagtgacatcatataataagggtgacattcttccagctttcaacaatgcagcacaggtatggaaccatgacttcttctgggagtgcatgaaaccaggtggaggtggaaagccatccggggagcttctagaactgattgaaagagactttggctcatttgaaaaattccttgatgagtttaaggctgctgctgcaacacaatttggttcaggatgggcttggcttgcatatagagcaagcaaatttgatggggaaaatgcagcaaatcctccttcaccagatgaggacaacaagctagtggtgctcaagagtcccaatgctgtgaacccccttgtttggggaggctattatccacttcttaccattgatgtttgggagcatgcttacaaccttgattttcagaaccggcgccctgattatatatcagtgttcatggataagcttgtttcctgggatgcagtgagttctagacttgaacaagcaaggctt

SEQ ID NO: 613 - protein, 244 aa, Glycine max
MASLGGLQNLSGINFLIKKEGPKVNAKFELKPPPYPLNGLEPVMSQQTLEFHWGKHHKTYVENLKKQIVGTELDGKSLEEIIVTSYNKGDILPAFNNAAQVWNHDFFWECMKPGGGGKPSGELLELIERDFGSFEKFLDEFKAAAATQFGSGWAWLAYRASKFDGENAANPPSPDEDNKLVVLKSPNAVNPLVWGGYYPLLTIDVWEHAYNLDFQNRRPDYISVFMDKLVSWDAVSSRLEQARL

SEQ ID NO: 614 - protein, 10 aa, Glycine max
QIVGTELDGK

SEQ ID NO: 615 - protein, 12 aa, Glycine max
SLEEIIVTSYNK

SEQ ID NO: 616 - protein, 7 aa, Glycine max
TYVENLK

SEQ ID NO: 617 - DNA, 1419 bp, Glycine max
  source 1..1419: mol_type = other DNA
atggcttctaaggtggtttcggttttggttatagcgatgatgttgtttgccatgaactgtaactgcactagtgttggccacatgccttcaaccaaagaggaaggtcacgattttcaggagtctaaagccaagaccacacaaactgcgaacaaggccatggaaactggtaaagagggacaagaagccgcagagtcatggacagaatgggctaaagagaaactcagtgaaggtcttggcttcaaacatgatcaggaatccaaagaatccaccactaacaaagtctctgattatgccactgacactgcacagaaatccaaggattatgcaactgacactgctcagaaatcaaaggactatgcaggtgatgctgctcagaaatcaaaggactacgcaggtgatgctgctcagaaaaccaaagactatgccagtgacactgcacagacatcaaaggactatgctggtgatgctgctcagaaatcaaagggctatgttggtgatgctgcacaaaagaccaaagaatatgttggtgatgcagcacagaagacaaaggattatgccactgacgcagcacagaagaccaaagattatgctactcagaagaccaaggactatgcaagtgatgcaactgatgctgcaaagaagactaaagattatgctgctcagaagactaaggactatgcaagtgaagctagtgatgttgcacagaacacaaaagattatgctgctcagaagaccaaggactatgccagcggtggtgcgcagaagaccaaggattatgccagcggtggtgcgcagaagaccaaggactatgcaagtgatgctgcgcagaagaccaaggactatgcaagtgatggtgctcaaaagagcaaagaatatgcgggtgatgtagcactgaatgccaaagattatgctcaaaaaagcaaagattacgcaggtgatgctgcacagaatgtcaaagattatgcaagtgatgcagtgcagaagagaaaagagtattctggtgatgcttctcacaagagcaaagaggcttctgattatgccagtgagacggctaagaagaccaaagattatgtcggtgatgctgctcagagaagcaaaggggctgctgaatatgctagtgatgcggctcagaggaccaaagagtatgctggtgatgctaccaagagaagcaaagaggcttctaatgatcatgccaatgacatggctcagaagaccaaagactacgccagtgacactgctcagagaaccaaggaaaaactccaagacattgcatctgaagctggtcaatacagtgcagagaaagctagagaaatgaaggatgcagcagcagagaaggcaagtgacatagcaaaggctgcaaagcaaaaatcacaagaggtgaaagagaaattaggtggacaacaccgggatgccgagctt

SEQ ID NO: 618 - protein, 473 aa, Glycine max
MASKVVSVLVIAMMLFAMNCNCTSVGHMPSTKEEGHDFQESKAKTTQTANKAMETGKEGQEAAESWTEWAKEKLSEGLGFKHDQESKESTTNKVSDYATDTAQKSKDYATDTAQKSKDYAGDAAQKSKDYAGDAAQKTKDYASDTAQTSKDYAGDAAQKSKGYVGDAAQKTKEYVGDAAQKTKDYATDAAQKTKDYATQKTKDYASDATDAAKKTKDYAAQKTKDYASEASDVAQNTKDYAAQKTKDYASGGAQKTKDYASGGAQKTKDYASDAAQKTKDYASDGAQKSKEYAGDVALNAKDYAQKSKDYAGDAAQNVKDYASDAVQKRKEYSGDASHKSKEASDYASETAKKTKDYVGDAAQRSKGAAEYASDAAQRTKEYAGDATKRSKEASNDHANDMAQKTKDYASDTAQRTKEKLQDIASEAGQYSAEKAREMKDAAAEKASDIAKAAKQKSQEVKEKLGGQHRDAEL

SEQ ID NO: 619 - protein, 11 aa, Glycine max
DYAGDAAQNVK

SEQ ID NO: 620 - protein, 9 aa, Glycine max
DYASDAAQK

SEQ ID NO: 621 - protein, 11 aa, Glycine max
DYASDATDAAK

SEQ ID NO: 622 - protein, 9 aa, Glycine max
DYASDAVQK

SEQ ID NO: 623 - protein, 11 aa, Glycine max
DYASDTAQRTK

SEQ ID NO: 624 - protein, 11 aa, Glycine max
DYASDTAQTSK

SEQ ID NO: 625 - protein, 14 aa, Glycine max
DYASEASDVAQNTK

SEQ ID NO: 626 - protein, 9 aa, Glycine max
DYATDAAQK

SEQ ID NO: 627 - protein, 11 aa, Glycine max
DYVGDAAQRSK

SEQ ID NO: 628 - protein, 11 aa, Glycine max
EASDYASETAK

SEQ ID NO: 629 - protein, 11 aa, Glycine max
EYAGDVALNAK

SEQ ID NO: 630 - protein, 9 aa, Glycine max
EYVGDAAQK

SEQ ID NO: 631 - protein, 14 aa, Glycine max
GAAEYASDAAQRTK

SEQ ID NO: 632 - protein, 9 aa, Glycine max
GYVGDAAQK

SEQ ID NO: 633 - protein, 15 aa, Glycine max
LQDIASEAGQYSAEK

SEQ ID NO: 634 - protein, 8 aa, Glycine max
LSEGLGFK

SEQ ID NO: 635 - protein, 11 aa, Glycine max
VSDYATDTAQK

SEQ ID NO: 636 - DNA, 1374 bp, Glycine max
  source 1..1374: mol_type = other DNA
atggcttcgagggtggtttcggttttggtgatagcgatgatgttgtttgccatgaactgtaactgcactagtgttggccacatgccttcaaccaaagaggaaggtcacgattttcaggaggctaaagccaagacaacacaaactgcgaacaaggccatggaaactggtaaagatgggaaagaagccgcagagtcatggacagaatgggctaaagagaaactcactgaaggtcttggcttcaaacatgatcaagaatccaccactaagaaagtctctgactatgccggtgatgctgctcagaaatcaaaggactatgccggtgacactgctcagaaaaccaaggactatgccggtgacactgctcagaaaaccaaggactatgccggtgacactgcacagacatcaaaggactatgccggtgatgctgctcagaaatcaaagggctatgtcggtgatgctgcacaaaagaccaaagaatatgttggtgatgctgctcagaagaccatggattatgccactgatgcagcacagaagaccaaagattatgctactcagaagactaaggactatgcaagtgatgcaactgatgctgcaaaaaagactaaagattatgctgctcagaagaccaaggattatgcaagtgatgcaactgatgctgcaaaaaagactaaggattatgctgcgcagaagaccaaggactatgccagtggtggtgcgcagaagaccaaggactatgccagtggtgctgcgcagaagaccaaggactatgcaagtgatgctgcgcagaggaccaaggaccatgcaagtgatggtgctcaaaagagcaaagaatatgcgggtgatgtagcacagaatgccaaagattatgctcaaaagagcaaagattatgcgggtgatgctgtacagaatatcaaagattatgcaaatgatgcagcgcacaagagcaaagattatgctggtgatgcttctcacaagagcaaagaagcttctgattatgccagtgagacggctcagaagaccaaagactatgttggtgatgctgctcagaaaagcaaagaggcttctgaatatgctagtgatgcggctcagaggaccaaagagtatgctggtgatgccaccaagagaagcaaagaggcttctgatcatgccaatgacatggctcggaagaccaaagactacgccagtgacactgctcagagaaccaaggaaaaactccaagacattgcatctgaggctggtcaatacagtacagagaaggccagagaaatgaaggatgcagcagcagagaaggctagtgacattgcaaaggctgcaaagcaaaaatcacaagagctgaaagacaaattaggtggacaacaccgggatgctgagctt

SEQ ID NO: 637 - protein, 458 aa, Glycine max
MASRVVSVLVIAMMLFAMNCNCTSVGHMPSTKEEGHDFQEAKAKTTQTANKAMETGKDGKEAAESWTEWAKEKLTEGLGFKHDQESTTKKVSDYAGDAAQKSKDYAGDTAQKTKDYAGDTAQKTKDYAGDTAQTSKDYAGDAAQKSKGYVGDAAQKTKEYVGDAAQKTMDYATDAAQKTKDYATQKTKDYASDATDAAKKTKDYAAQKTKDYASDATDAAKKTKDYAAQKTKDYASGGAQKTKDYASGAAQKTKDYASDAAQRTKDHASDGAQKSKEYAGDVAQNAKDYAQKSKDYAGDAVQNIKDYANDAAHKSKDYAGDASHKSKEASDYASETAQKTKDYVGDAAQKSKEASEYASDAAQRTKEYAGDATKRSKEASDHANDMARKTKDYASDTAQRTKEKLQDIASEAGQYSTEKAREMKDAAAEKASDIAKAAKQKSQELKDKLGGQHRDAEL

SEQ ID NO: 638 - protein, 11 aa, Glycine max
DYAGDAVQNIK

SEQ ID NO: 639 - protein, 11 aa, Glycine max
DYASDAAQRTK

SEQ ID NO: 640 - protein, 11 aa, Glycine max
DYASDATDAAK

SEQ ID NO: 641 - protein, 11 aa, Glycine max
DYASDTAQRTK

SEQ ID NO: 642 - protein, 9 aa, Glycine max
DYVGDAAQK

SEQ ID NO: 643 - protein, 12 aa, Glycine max
EASDYASETAQK

SEQ ID NO: 644 - protein, 14 aa, Glycine max
EASEYASDAAQRTK

SEQ ID NO: 645 - protein, 11 aa, Glycine max
EYAGDVAQNAK

SEQ ID NO: 646 - protein, 9 aa, Glycine max
GYVGDAAQK

SEQ ID NO: 647 - protein, 15 aa, Glycine max
LQDIASEAGQYSTEK

SEQ ID NO: 648 - protein, 8 aa, Glycine max
LTEGLGFK

SEQ ID NO: 649 - protein, 11 aa, Glycine max
VSDYAGDAAQK

SEQ ID NO: 650-651 - skipped (2 entries)

SEQ ID NO: 652 - protein, 8 aa, synthetic construct
  REGION 1..8: note = epitope
STKSEGKL

SEQ ID NO: 653 - protein, 9 aa, synthetic construct
  REGION 1..9: note = epitope
  VARIANT 1..3: note = any amino acid
  VARIANT 8..9: note = any amino acid
XXXKDNVXX

SEQ ID NO: 654 - protein, 9 aa, synthetic construct
  REGION 1..9: note = epitope
  VARIANT 1: note = any amino acid
  VARIANT 3: note = any amino acid
  VARIANT 8..9: note = any amino acid
XLXKDNVXX

SEQ ID NO: 655 - protein, 9 aa, synthetic construct
  REGION 1..9: note = epitope
ALSKDNVDD

SEQ ID NO: 656 - protein, 15 aa, synthetic construct
  REGION 1..15: note = epitope
  VARIANT 6..10: note = any amino acid
  VARIANT 12..15: note = any amino acid
EEEDDXXXXXEXXXX

SEQ ID NO: 657 - protein, 15 aa, synthetic construct
  REGION 1..15: note = epitope
EEEDDTEVEAESDAK

SEQ ID NO: 658 - protein, 15 aa, synthetic construct
  REGION 1..15: note = epitope
LHLKEEAGEYLEESK

SEQ ID NO: 659 - protein, 15 aa, synthetic construct
  REGION 1..15: note = epitope
SLDISPEATVEEEDD

SEQ ID NO: 660 - protein, 10 aa, synthetic construct
  REGION 1..10: note = epitope
ESTDKEKKDA

SEQ ID NO: 661 - skipped

SEQ ID NO: 662 - protein, 9 aa, synthetic construct
  REGION 1..9: note = epitope
  VARIANT 2..3: note = any amino acid
  VARIANT 6: note = any amino acid
  VARIANT 8..9: note = any amino acid
DXXDKXRXX

SEQ ID NO: 663 - protein, 9 aa, synthetic construct
  REGION 1..9: note = epitope
DALDKIRFE

SEQ ID NO: 664 - protein, 15 aa, synthetic construct
  REGION 1..15: note = epitope
  VARIANT 1..4: note = any amino acid
  VARIANT 9..15: note = any amino acid
XXXXKSKLXXXXXXX

SEQ ID NO: 665 - protein, 15 aa, synthetic construct
  REGION 1..15: note = epitope
  VARIANT 1..4: note = any amino acid
  VARIANT 9..11: note = any amino acid
  VARIANT 13..15: note = any amino acid
XXXXKSKLXXXPXXX

SEQ ID NO: 666 - protein, 15 aa, synthetic construct
  REGION 1..15: note = epitope
SLTDKSKLDAQPELF

SEQ ID NO: 667 - skipped

SEQ ID NO: 668 - protein, 5 aa, synthetic construct
  REGION 1..5: note = epitope
  VARIANT 5: note = any amino acid
IPDKX

SEQ ID NO: 669 - protein, 5 aa, synthetic construct
  REGION 1..5: note = epitope
IPDKT

SEQ ID NO: 670 - protein, 15 aa, synthetic construct
  REGION 1..15: note = epitope
DDEDEEEKKDEEGKV

SEQ ID NO: 671 - protein, 15 aa, synthetic construct
  REGION 1..15: note = epitope
PEYLSFVKGIVDSED

SEQ ID NO: 672 - protein, 15 aa, synthetic construct
  REGION 1..15: note = epitope
VDSEDLPLNISREML

SEQ ID NO: 673 - protein, 15 aa, synthetic construct
  REGION 1..15: note = epitope
  VARIANT 1..2: note = any amino acid
  VARIANT 6..12: note = any amino acid
  VARIANT 15: note = any amino acid
XXFSKXXXXXXXEDX

SEQ ID NO: 674 - protein, 15 aa, synthetic construct
  REGION 1..15: note = epitope
  VARIANT 1..2: note = any amino acid
  VARIANT 6..12: note = any amino acid
XXFSKXXXXXXXEDS

SEQ ID NO: 675 - protein, 15 aa, synthetic construct
  REGION 1..15: note = epitope
EAFSKNLKLGIHEDS

SEQ ID NO: 676 - protein, 15 aa, synthetic construct
  REGION 1..15: note = epitope
LGDKVEKVVVSDRVV

SEQ ID NO: 677 - protein, 15 aa, synthetic construct
  REGION 1..15: note = epitope
DEDAGEADADMPPLE

SEQ ID NO: 678 - protein, 15 aa, synthetic construct
  REGION 1..15: note = epitope
MPPLEDADADAEGSK

SEQ ID NO: 679 - skipped

SEQ ID NO: 680 - protein, 15 aa, synthetic construct
  REGION 1..15: note = epitope
  VARIANT 1..4: note = any amino acid
  VARIANT 9: note = any amino acid
  VARIANT 11..13: note = any amino acid
XXXXDKIRXEXXXDK

SEQ ID NO: 681 - protein, 15 aa, synthetic construct
  REGION 1..15: note = epitope
SDALDKIRFESLTDK

SEQ ID NO: 682 - protein, 15 aa, synthetic construct
  REGION 1..15: note = epitope
  VARIANT 1: note = any amino acid
  VARIANT 3..4: note = any amino acid
  VARIANT 6..9: note = any amino acid
  VARIANT 12..15: note = any amino acid
XYXXRXXXXQNXXXX

SEQ ID NO: 683 - protein, 15 aa, synthetic construct
  REGION 1..15: note = epitope
  VARIANT 1: note = any amino acid
  VARIANT 3..4: note = any amino acid
  VARIANT 6..9: note = any amino acid
  VARIANT 12..15: note = any amino acid
XYXXRXXXXQNXXXX

SEQ ID NO: 684 - protein, 15 aa, synthetic construct
  REGION 1..15: note = epitope
DYVTRMKEGQNDIYY

SEQ ID NO: 685 - protein, 15 aa, synthetic construct
  REGION 1..15: note = epitope
  VARIANT 2..4: note = any amino acid
  VARIANT 6..9: note = any amino acid
  VARIANT 12..15: note = any amino acid
DXXXRXXXXQNXXXX

SEQ ID NO: 686 - protein, 15 aa, synthetic construct
  REGION 1..15: note = epitope
  VARIANT 2..4: note = any amino acid
  VARIANT 6..9: note = any amino acid
  VARIANT 12..15: note = any amino acid
DXXXRXXXXQNXXXX

SEQ ID NO: 687 - protein, 15 aa, synthetic construct
  REGION 1..15: note = epitope
DYVTRMKEGQNDIYY

SEQ ID NO: 688 - protein, 15 aa, synthetic construct
  REGION 1..15: note = epitope
  VARIANT 4..11: note = any amino acid
  VARIANT 14..15: note = any amino acid
RKPXXXXXXXXAAXX

SEQ ID NO: 689 - protein, 15 aa, synthetic construct
  REGION 1..15: note = epitope
RKPEEITKEEYAAFY

SEQ ID NO: 690 - protein, 9 aa, synthetic construct
  REGION 1..9: note = epitope
SLTDKSKLD

SEQ ID NO: 691 - protein, 8 aa, synthetic construct
  REGION 1..8: note = epitope
FIHIIPDK

SEQ ID NO: 692 - protein, 8 aa, synthetic construct
  REGION 1..8: note = epitope
VTRMKEGQ

SEQ ID NO: 693 - skipped

SEQ ID NO: 694 - protein, 15 aa, synthetic construct
  REGION 1..15: note = epitope
  VARIANT 1..3: note = any amino acid
XXXLKREDEERLDEV

SEQ ID NO: 695 - protein, 15 aa, synthetic construct
  REGION 1..15: note = epitope
GEPLKREDEERLDEV

SEQ ID NO: 696 - skipped

SEQ ID NO: 697 - protein, 15 aa, synthetic construct
  REGION 1..15: note = epitope
ILERKKSPNRLVVDE

SEQ ID NO: 698 - skipped

SEQ ID NO: 699 - protein, 7 aa, synthetic construct
  REGION 1..7: note = epitope
FPESGDR

SEQ ID NO: 700 - protein, 7 aa, synthetic construct
  REGION 1..7: note = epitope
SDPKSGK

SEQ ID NO: 701 - protein, 8 aa, synthetic construct
  REGION 1..8: note = epitope
QTMEKLQL

SEQ ID NO: 702 - protein, 7 aa, synthetic construct
  REGION 1..7: note = epitope
LGDVVSV

SEQ ID NO: 703 - protein, 15 aa, synthetic construct
  REGION 1..15: note = epitope
GGMRSVEFKVVETDP

SEQ ID NO: 704 - protein, 15 aa, synthetic construct
  REGION 1..15: note = epitope
RKAFEEAEKNAPSII

SEQ ID NO: 705 - protein, 8 aa, synthetic construct
  REGION 1..8: note = epitope
KREKTHGE

SEQ ID NO: 706 - protein, 7 aa, synthetic construct
  REGION 1..7: note = epitope
RIVSQLL

SEQ ID NO: 707 - protein, 15 aa, synthetic construct
  REGION 1..15: note = epitope
SRAHVIVIGATNRPN

SEQ ID NO: 708 - protein, 9 aa, synthetic construct
  REGION 1..9: note = epitope
LRRFGRFDR

SEQ ID NO: 709 - protein, 15 aa, synthetic construct
  REGION 1..15: note = epitope
MDVIDLEDETIDAEV

SEQ ID NO: 710 - protein, 15 aa, synthetic construct
  REGION 1..15: note = epitope
VTNEHFQTALGTSNP

SEQ ID NO: 711 - protein, 15 aa, synthetic construct
  REGION 1..15: note = epitope
VEHPEKFEKFGMSPS

SEQ ID NO: 712 - protein, 8 aa, synthetic construct
  REGION 1..8: note = epitope
EIFDKARQ

SEQ ID NO: 713 - protein, 15 aa, synthetic construct
  REGION 1..15: note = epitope
DIIDPALLRPGRLDQ

SEQ ID NO: 714 - protein, 10 aa, synthetic construct
  REGION 1..10: note = epitope
IRENIEKDIE

SEQ ID NO: 715 - protein, 15 aa, synthetic construct
  REGION 1..15: note = epitope
IERERKSRENPEAMD

SEQ ID NO: 716 - protein, 15 aa, synthetic construct
  REGION 1..15: note = epitope
PEAMDEDTVDDEVAE

SEQ ID NO: 717 - skipped

SEQ ID NO: 718 - protein, 10 aa, synthetic construct
  REGION 1..10: note = epitope
  VARIANT 1..5: note = any amino acid
  VARIANT 7: note = any amino acid
XXXXXEXKED

SEQ ID NO: 719 - protein, 10 aa, synthetic construct
  REGION 1..10: note = epitope
LDKFAENKED

SEQ ID NO: 720 - protein, 7 aa, synthetic construct
  REGION 1..7: note = epitope
  VARIANT 2..3: note = any amino acid
  VARIANT 7: note = any amino acid
KXXLGDX

SEQ ID NO: 721 - protein, 7 aa, synthetic construct
  REGION 1..7: note = epitope
  VARIANT 3: note = any amino acid
  VARIANT 7: note = any amino acid
KIXLGDX

SEQ ID NO: 722 - protein, 7 aa, synthetic construct
  REGION 1..7: note = epitope
KIRLGDT

SEQ ID NO: 723 - protein, 10 aa, synthetic construct
  REGION 1..10: note = epitope
  VARIANT 1..3: note = any amino acid
  VARIANT 5: note = any amino acid
  VARIANT 7: note = any amino acid
  VARIANT 9: note = any amino acid
XXXDXFXKXE

SEQ ID NO: 724 - protein, 10 aa, synthetic construct
  REGION 1..10: note = epitope
  VARIANT 1..3: note = any amino acid
  VARIANT 5: note = any amino acid
  VARIANT 7: note = any amino acid
XXXDXFXKIE

SEQ ID NO: 725 - protein, 10 aa, synthetic construct
  REGION 1..10: note = epitope
GDTDLFAKIE

SEQ ID NO: 726 - protein, 15 aa, synthetic construct
  REGION 1..15: note = epitope
IDSCLTVADQYDIQI

SEQ ID NO: 727 - protein, 15 aa, synthetic construct
  REGION 1..15: note = epitope
NPTRPLTLNTIDEHL

SEQ ID NO: 728 - protein, 15 aa, synthetic construct
  REGION 1..15: note = epitope
CHHLNREIPEDLAFA

SEQ ID NO: 729 - protein, 15 aa, synthetic construct
  REGION 1..15: note = epitope
  VARIANT 1..3: note = any amino acid
  VARIANT 5: note = any amino acid
  VARIANT 7..9: note = any amino acid
  VARIANT 12..13: note = any amino acid
XXXHXLXXXNKXXEA

SEQ ID NO: 730 - protein, 15 aa, synthetic construct
  REGION 1..15: note = epitope
  VARIANT 1..2: note = any amino acid
  VARIANT 5: note = any amino acid
  VARIANT 7..9: note = any amino acid
  VARIANT 12..13: note = any amino acid
XXVHXLXXXNKXXEA

SEQ ID NO: 731 - protein, 15 aa, synthetic construct
  REGION 1..15: note = epitope
QRVHALYGLNKRVEA

SEQ ID NO: 732 - protein, 15 aa, synthetic construct
  REGION 1..15: note = epitope
  VARIANT 1..6: note = any amino acid
  VARIANT 9: note = any amino acid
  VARIANT 11: note = any amino acid
  VARIANT 13: note = any amino acid
  VARIANT 15: note = any amino acid
XXXXXXGLXKXRXEX

SEQ ID NO: 733 - protein, 15 aa, synthetic construct
  REGION 1..15: note = epitope
  VARIANT 1..6: note = any amino acid
  VARIANT 9: note = any amino acid
  VARIANT 11: note = any amino acid
  VARIANT 13: note = any amino acid
XXXXXXGLXKXRXEG

SEQ ID NO: 734 - protein, 15 aa, synthetic construct
  REGION 1..15: note = epitope
KAHEIAGLRKERAEG

SEQ ID NO: 735 - protein, 15 aa, synthetic construct
  REGION 1..15: note = epitope
ERAEGKFVPDPRFEE

SEQ ID NO: 736 - protein, 15 aa, synthetic construct
  REGION 1..15: note = epitope
RSGIFGSYNYDELMG

SEQ ID NO: 737 - protein, 15 aa, synthetic construct
  REGION 1..15: note = epitope
DELMGSLEGNEGFGR

SEQ ID NO: 738 - protein, 15 aa, synthetic construct
  REGION 1..15: note = epitope
LNTAGSYKFSSDRTI

SEQ ID NO: 739 - protein, 15 aa, synthetic construct
  REGION 1..15: note = epitope
AAEKYKSEGHDMIIL

SEQ ID NO: 740 - protein, 15 aa, synthetic construct
  REGION 1..15: note = epitope
DSLGLTGHERYTIDL

SEQ ID NO: 741 - protein, 15 aa, synthetic construct
  REGION 1..15: note = epitope
IRKVVELQEELDIDV

SEQ ID NO: 742 - protein, 15 aa, synthetic construct
  REGION 1..15: note = epitope
  VARIANT 1..7: note = any amino acid
  VARIANT 10: note = any amino acid
  VARIANT 14: note = any amino acid
XXXXXXXKDXVEDXE

SEQ ID NO: 743 - protein, 15 aa, synthetic construct
  REGION 1..15: note = epitope
  VARIANT 1..7: note = any amino acid
  VARIANT 14: note = any amino acid
XXXXXXXKDEVEDXE

SEQ ID NO: 744 - protein, 15 aa, synthetic construct
  REGION 1..15: note = epitope
YQIALSIKDEVEDLE

SEQ ID NO: 745 - skipped

SEQ ID NO: 746 - protein, 10 aa, synthetic construct
  REGION 1..10: note = epitope
  VARIANT 2: note = any amino acid
  VARIANT 4..7: note = any amino acid
  VARIANT 10: note = any amino acid
NXEXXXXKDX

SEQ ID NO: 747 - protein, 10 aa, synthetic construct
  REGION 1..10: note = epitope
NPETYILKDQ

SEQ ID NO: 748 - protein, 8 aa, synthetic construct
  REGION 1..8: note = epitope
  VARIANT 6: note = any amino acid
LDFLEXET

SEQ ID NO: 749 - protein, 8 aa, synthetic construct
  REGION 1..8: note = epitope
LDFLEVET

SEQ ID NO: 750 - protein, 9 aa, synthetic construct
  REGION 1..9: note = epitope
QYTEGDKRV

SEQ ID NO: 751 - protein, 10 aa, synthetic construct
  REGION 1..10: note = epitope
  VARIANT 3: note = any amino acid
  VARIANT 6..10: note = any amino acid
PLXQQXXXXX

SEQ ID NO: 752 - protein, 10 aa, synthetic construct
  REGION 1..10: note = epitope
  VARIANT 3: note = any amino acid
  VARIANT 7..10: note = any amino acid
PLXQQGXXXX

SEQ ID NO: 753 - protein, 10 aa, synthetic construct
  REGION 1..10: note = epitope
PLVQQGQQGQ

SEQ ID NO: 754 - protein, 8 aa, synthetic construct
  REGION 1..8: note = epitope
LGEMNGKM

SEQ ID NO: 755 - protein, 15 aa, synthetic construct
  REGION 1..15: note = epitope
KVITLSVEDQDYMGR

SEQ ID NO: 756 - skipped

SEQ ID NO: 757 - protein, 15 aa, synthetic construct
  REGION 1..15: note = epitope
  VARIANT 1..2: note = any amino acid
  VARIANT 4: note = any amino acid
  VARIANT 6..9: note = any amino acid
  VARIANT 11: note = any amino acid
  VARIANT 14: note = any amino acid
XXGXLXXXXGXRTXE

SEQ ID NO: 758 - protein, 15 aa, synthetic construct
  REGION 1..15: note = epitope
ASGKLSQYEGGRTKE

SEQ ID NO: 759 - protein, 15 aa, synthetic construct
  REGION 1..15: note = epitope
  VARIANT 1..5: note = any amino acid
  VARIANT 8..11: note = any amino acid
XXXXXLLXXXXEEDN

SEQ ID NO: 760 - protein, 15 aa, synthetic construct
  REGION 1..15: note = epitope
  VARIANT 1..5: note = any amino acid
  VARIANT 9..11: note = any amino acid
XXXXXLLEXXXEEDN

SEQ ID NO: 761 - protein, 15 aa, synthetic construct
  REGION 1..15: note = epitope
EKSDNLLEGGGEEDN

SEQ ID NO: 762 - protein, 15 aa, synthetic construct
  REGION 1..15: note = epitope
  VARIANT 3..11: note = any amino acid
  VARIANT 13: note = any amino acid
  VARIANT 15: note = any amino acid
RKXXXXXXXXXYXNX

SEQ ID NO: 763 - protein, 15 aa, synthetic construct
  REGION 1..15: note = epitope
  VARIANT 6..10: note = any amino acid
  VARIANT 15: note = any amino acid
RKDHSXXXXXLYANX

SEQ ID NO: 764 - protein, 15 aa, synthetic construct
  REGION 1..15: note = epitope
RKDHSDVSNQLYANY

SEQ ID NO: 765 - skipped

SEQ ID NO: 766 - protein, 15 aa, synthetic construct
  REGION 1..15: note = epitope
QGKEYVFVANSDNLG

SEQ ID NO: 767 - protein, 15 aa, synthetic construct
  REGION 1..15: note = epitope
IAKYGLDDNTIDFIG

SEQ ID NO: 768 - protein, 15 aa, synthetic construct
  REGION 1..15: note = epitope
EETREKKEEESRERE

SEQ ID NO: 769 - protein, 15 aa, synthetic construct
  REGION 1..15: note = epitope
  VARIANT 1..7: note = any amino acid
  VARIANT 9..10: note = any amino acid
XXXXXXXNXXIFEED

SEQ ID NO: 770 - protein, 15 aa, synthetic construct
  REGION 1..15: note = epitope
  VARIANT 1..7: note = any amino acid
  VARIANT 9: note = any amino acid
XXXXXXXNXYIFEED

SEQ ID NO: 771 - protein, 15 aa, synthetic construct
  REGION 1..15: note = epitope
QHEEQDENPYIFEED

SEQ ID NO: 772 - protein, 15 aa, synthetic construct
  REGION 1..15: note = epitope
SIFAISREQVRALAP

SEQ ID NO: 773 - protein, 10 aa, synthetic construct
  REGION 1..10: note = epitope
  VARIANT 1: note = any amino acid
  VARIANT 7: note = any amino acid
  VARIANT 9..10: note = any amino acid
XELPREXRXX

SEQ ID NO: 774 - protein, 10 aa, synthetic construct
  REGION 1..10: note = epitope
  VARIANT 1: note = any amino acid
  VARIANT 9..10: note = any amino acid
XELPREERXX

SEQ ID NO: 775 - protein, 10 aa, synthetic construct
  REGION 1..10: note = epitope
FELPREERGR

SEQ ID NO: 776 - protein, 15 aa, synthetic construct
  REGION 1..15: note = epitope
  VARIANT 1..5: note = any amino acid
  VARIANT 9..12: note = any amino acid
  VARIANT 15: note = any amino acid
XXXXXPLYXXXXDEX

SEQ ID NO: 777 - protein, 15 aa, synthetic construct
  REGION 1..15: note = epitope
  VARIANT 1..5: note = any amino acid
  VARIANT 10..12: note = any amino acid
  VARIANT 15: note = any amino acid
XXXXXPLYIXXXDEX

SEQ ID NO: 778 - protein, 15 aa, synthetic construct
  REGION 1..15: note = epitope
IPAGTPLYIVNRDEN

SEQ ID NO: 779 - skipped

SEQ ID NO: 780 - protein, 15 aa, synthetic construct
  REGION 1..15: note = epitope
  VARIANT 1..5: note = any amino acid
  VARIANT 7..8: note = any amino acid
  VARIANT 10: note = any amino acid
  VARIANT 13..15: note = any amino acid
XXXXXSXXRXRRXXX

SEQ ID NO: 781 - protein, 15 aa, synthetic construct
  REGION 1..15: note = epitope
PFELPSEERGRRAVA

SEQ ID NO: 782 - skipped

SEQ ID NO: 783 - protein, 15 aa, synthetic construct
  REGION 1..15: note = epitope
  VARIANT 1: note = any amino acid
  VARIANT 9..10: note = any amino acid
  VARIANT 15: note = any amino acid
XKEEEHQEXXEEEQX

SEQ ID NO: 784 - protein, 15 aa, synthetic construct
  REGION 1..15: note = epitope
EKEEEHQEQHEEEQD

SEQ ID NO: 785 - skipped

SEQ ID NO: 786 - protein, 15 aa, synthetic construct
  REGION 1..15: note = epitope
FAGKDNIVSSLDNVA

SEQ ID NO: 787 - protein, 10 aa, synthetic construct
  REGION 1..10: note = epitope
SNLALGKLKE

SEQ ID NO: 788 - protein, 15 aa, synthetic construct
  REGION 1..15: note = epitope
EGGEKGNKDRPELKT

SEQ ID NO: 789 - protein, 8 aa, synthetic construct
  REGION 1..8: note = epitope
LNTTTQTA

SEQ ID NO: 790 - protein, 15 aa, synthetic construct
  REGION 1..15: note = epitope
QQGYAVTKDTISSAA

SEQ ID NO: 791 - protein, 8 aa, synthetic construct
  REGION 1..8: note = epitope
KKPSQPQE

SEQ ID NO: 792 - protein, 15 aa, synthetic construct
  REGION 1..15: note = epitope
IGEKLGDAKQREELL

SEQ ID NO: 793 - protein, 5 aa, synthetic construct
  REGION 1..5: note = epitope
ETKDT

SEQ ID NO: 794 - protein, 8 aa, synthetic construct
  REGION 1..8: note = epitope
VAYPLDLF

SEQ ID NO: 795 - protein, 9 aa, synthetic construct
  REGION 1..9: note = epitope
KDDENVNSQ

SEQ ID NO: 796 - protein, 15 aa, synthetic construct
  REGION 1..15: note = epitope
CAEAIFKSQAETGEI

SEQ ID NO: 797 - protein, 9 aa, synthetic construct
  REGION 1..9: note = epitope
GHYLNATAG

SEQ ID NO: 798 - protein, 5 aa, synthetic construct
  REGION 1..5: note = epitope
LHIHR

SEQ ID NO: 799 - protein, 15 aa, synthetic construct
  REGION 1..15: note = epitope
DDFVEKDRSRGIYFT

SEQ ID NO: 800 - protein, 7 aa, synthetic construct
  REGION 1..7: note = epitope
WGNAPGA

SEQ ID NO: 801 - protein, 15 aa, synthetic construct
  REGION 1..15: note = epitope
RNEGRDLAREGNEII

SEQ ID NO: 802 - protein, 15 aa, synthetic construct
  REGION 1..15: note = epitope
VWKEIKFEFEAMDTL

SEQ ID NO: 803 - protein, 15 aa, synthetic construct
  REGION 1..15: note = epitope
HPGDGASGRLSNATG

SEQ ID NO: 804 - protein, 15 aa, synthetic construct
  REGION 1..15: note = epitope
VIRIVSEQTDILEPF

SEQ ID NO: 805 - protein, 15 aa, synthetic construct
  REGION 1..15: note = epitope
ATSGDRDGKTFVSTA

SEQ ID NO: 806 - protein, 15 aa, synthetic construct
  REGION 1..15: note = epitope
PVTVNLWQPEKNAFE

SEQ ID NO: 807 - protein, 7 aa, synthetic construct
  REGION 1..7: note = epitope
AIRVTQS

SEQ ID NO: 808 - protein, 15 aa, synthetic construct
  REGION 1..15: note = epitope
SEARKSTNTPDAQKV

SEQ ID NO: 809 - protein, 15 aa, synthetic construct
  REGION 1..15: note = epitope
ILWHLTKEDKTYGVP

SEQ ID NO: 810 - protein, 15 aa, synthetic construct
  REGION 1..15: note = epitope
VAFSIDNRQIVSASR

SEQ ID NO: 811 - protein, 9 aa, synthetic construct
  REGION 1..9: note = epitope
RLYSLDAGS

SEQ ID NO: 812 - protein, 9 aa, synthetic construct
  REGION 1..9: note = epitope
DLKVDLKTE

SEQ ID NO: 813 - protein, 15 aa, synthetic construct
  REGION 1..15: note = epitope
KSGEERAQEEAIDID

SEQ ID NO: 814 - protein, 15 aa, synthetic construct
  REGION 1..15: note = epitope
DNQLVAADAKLNFDD

SEQ ID NO: 815 - protein, 15 aa, synthetic construct
  REGION 1..15: note = epitope
FLDVGGNASENQVVE

SEQ ID NO: 816 - protein, 15 aa, synthetic construct
  REGION 1..15: note = epitope
VRFYKEEEKNDPEFA

SEQ ID NO: 817 - skipped

SEQ ID NO: 818 - protein, 7 aa, synthetic construct
  REGION 1..7: note = epitope
  VARIANT 5..7: note = any amino acid
FDKFXXX

SEQ ID NO: 819 - protein, 7 aa, synthetic construct
  REGION 1..7: note = epitope
FDKFATG

SEQ ID NO: 820 - protein, 15 aa, synthetic construct
  REGION 1..15: note = epitope
GKPLVIEEVEVAPPQ

SEQ ID NO: 821 - protein, 10 aa, synthetic construct
  REGION 1..10: note = epitope
  VARIANT 1: note = any amino acid
  VARIANT 3..6: note = any amino acid
XQXXXXENKP

SEQ ID NO: 822 - protein, 10 aa, synthetic construct
  REGION 1..10: note = epitope
  VARIANT 3: note = any amino acid
  VARIANT 5..6: note = any amino acid
EQXQXXENKP

SEQ ID NO: 823 - protein, 10 aa, synthetic construct
  REGION 1..10: note = epitope
EQHQHDENKP

SEQ ID NO: 824 - skipped

SEQ ID NO: 825 - protein, 15 aa, synthetic construct
  REGION 1..15: note = epitope
  VARIANT 1: note = any amino acid
  VARIANT 4..6: note = any amino acid
  VARIANT 9..13: note = any amino acid
  VARIANT 15: note = any amino acid
XKDXXXEKXXXXXDX

SEQ ID NO: 826 - protein, 15 aa, synthetic construct
  REGION 1..15: note = epitope
YKDYTAEKAIEGKDS

SEQ ID NO: 827 - protein, 15 aa, synthetic construct
  REGION 1..15: note = epitope
  VARIANT 1..3: note = any amino acid
  VARIANT 5..6: note = any amino acid
  VARIANT 10..13: note = any amino acid
  VARIANT 15: note = any amino acid
XXXDXXREEXXXXRX

SEQ ID NO: 828 - protein, 15 aa, synthetic construct
  REGION 1..15: note = epitope
  VARIANT 1..3: note = any amino acid
  VARIANT 5..6: note = any amino acid
  VARIANT 12..13: note = any amino acid
  VARIANT 15: note = any amino acid
XXXDXXREEEEXXRX

SEQ ID NO: 829 - protein, 15 aa, synthetic construct
  REGION 1..15: note = epitope
GNKDQTREEEEETRR

SEQ ID NO: 830 - protein, 15 aa, synthetic construct
  REGION 1..15: note = epitope
KETKMGEYSDYATQK

SEQ ID NO: 831 - protein, 15 aa, synthetic construct
  REGION 1..15: note = epitope
QKAGEYTDYAAQKAK

SEQ ID NO: 832 - protein, 15 aa, synthetic construct
  REGION 1..15: note = epitope
TTVQKAGEYKDYTAE

SEQ ID NO: 833 - protein, 15 aa, synthetic construct
  REGION 1..15: note = epitope
KDTTVQKAGEYKDYT

SEQ ID NO: 834 - protein, 15 aa, synthetic construct
  REGION 1..15: note = epitope
DGDMEEEGVLHVERR

SEQ ID NO: 835 - protein, 15 aa, synthetic construct
  REGION 1..15: note = epitope
EEEGVLHVERRREKM

SEQ ID NO: 836 - skipped

SEQ ID NO: 837 - protein, 10 aa, synthetic construct
  REGION 1..10: note = epitope
  VARIANT 1..5: note = any amino acid
  VARIANT 7: note = any amino acid
XXXXXFXDKP

SEQ ID NO: 838 - protein, 10 aa, synthetic construct
  REGION 1..10: note = epitope
EKTLLFGDKP

SEQ ID NO: 839 - protein, 15 aa, synthetic construct
  REGION 1..15: note = epitope
KLKGILGYTEDDVVS

SEQ ID NO: 840 - skipped

SEQ ID NO: 841 - protein, 15 aa, synthetic construct
  REGION 1..15: note = epitope
  VARIANT 2..11: note = any amino acid
  VARIANT 14: note = any amino acid
LXXXXXXXXXXDDXE

SEQ ID NO: 842 - protein, 15 aa, synthetic construct
  REGION 1..15: note = epitope
LEFTQGDLRNRDDLE

SEQ ID NO: 843 - protein, 15 aa, synthetic construct
  REGION 1..15: note = epitope
SVSIIDNFDNSVVEA

SEQ ID NO: 844 - protein, 15 aa, synthetic construct
  REGION 1..15: note = epitope
EKIPCEEDFRLQAMN

SEQ ID NO: 845 - protein, 15 aa, synthetic construct
  REGION 1..15: note = epitope
PELNVYGHDYPTRDG

SEQ ID NO: 846 - protein, 15 aa, synthetic construct
  REGION 1..15: note = epitope
PTRDGSAIRDYIHVM

SEQ ID NO: 847 - protein, 15 aa, synthetic construct
  REGION 1..15: note = epitope
  VARIANT 1..4: note = any amino acid
  VARIANT 7..9: note = any amino acid
  VARIANT 12..14: note = any amino acid
XXXXDKXXXERXXXE

SEQ ID NO: 848 - protein, 15 aa, synthetic construct
  REGION 1..15: note = epitope
  VARIANT 1..4: note = any amino acid
  VARIANT 9: note = any amino acid
  VARIANT 12..14: note = any amino acid
XXXXDKRVXERXXXE

SEQ ID NO: 849 - protein, 15 aa, synthetic construct
  REGION 1..15: note = epitope
LGGIDKRVIERFEKE

SEQ ID NO: 850 - protein, 15 aa, synthetic construct
  REGION 1..15: note = epitope
GGFEAGISKDGQTRE

SEQ ID NO: 851 - protein, 9 aa, synthetic construct
  REGION 1..9: note = epitope
  VARIANT 4: note = any amino acid
  VARIANT 7..9: note = any amino acid
PISXFEXXX

SEQ ID NO: 852 - protein, 9 aa, synthetic construct
  REGION 1..9: note = epitope
  VARIANT 7..9: note = any amino acid
PISGFEXXX

SEQ ID NO: 853 - protein, 9 aa, synthetic construct
  REGION 1..9: note = epitope
PISGFEGDN

SEQ ID NO: 854 - skipped

SEQ ID NO: 855 - protein, 10 aa, synthetic construct
  REGION 1..10: note = epitope
  VARIANT 4: note = any amino acid
  VARIANT 7..10: note = any amino acid
LLEXLDXXXX

SEQ ID NO: 856 - protein, 10 aa, synthetic construct
  REGION 1..10: note = epitope
LLEALDQINE

SEQ ID NO: 857 - protein, 15 aa, synthetic construct
  REGION 1..15: note = epitope
GTVEDIDEPRLERVF

SEQ ID NO: 858 - protein, 15 aa, synthetic construct
  REGION 1..15: note = epitope
WTPLIPASFKEEETA

SEQ ID NO: 859 - protein, 8 aa, synthetic construct
  REGION 1..8: note = epitope
  VARIANT 2..5: note = any amino acid
KXXXXQQQ

SEQ ID NO: 860 - protein, 8 aa, synthetic construct
  REGION 1..8: note = epitope
  VARIANT 2..4: note = any amino acid
KXXXQQQQ

SEQ ID NO: 861 - protein, 8 aa, synthetic construct
  REGION 1..8: note = epitope
KFPPQQQQ

SEQ ID NO: 862-863 - skipped (2 entries)

SEQ ID NO: 864 - protein, 7 aa, synthetic construct
  REGION 1..7: note = epitope
RGKVEAE

SEQ ID NO: 865 - skipped

SEQ ID NO: 866 - protein, 10 aa, synthetic construct
  REGION 1..10: note = epitope
  VARIANT 5..10: note = any amino acid
RRGKXXXXXX

SEQ ID NO: 867 - protein, 10 aa, synthetic construct
  REGION 1..10: note = epitope
RRGKESGGGG

SEQ ID NO: 868 - skipped

SEQ ID NO: 869 - protein, 15 aa, synthetic construct
  REGION 1..15: note = epitope
RNKQANKGNEEGRRE

SEQ ID NO: 870 - skipped

SEQ ID NO: 871 - protein, 15 aa, synthetic construct
  REGION 1..15: note = epitope
MASEQMPRRENNSNR

SEQ ID NO: 872 - protein, 15 aa, synthetic construct
  REGION 1..15: note = epitope
  VARIANT 1..3: note = any amino acid
  VARIANT 9: note = any amino acid
  VARIANT 11..15: note = any amino acid
XXXKGRVVXEXXXXX

SEQ ID NO: 873 - protein, 15 aa, synthetic construct
  REGION 1..15: note = epitope
  VARIANT 9: note = any amino acid
LPDKGRVVXESEKKE

SEQ ID NO: 874 - protein, 15 aa, synthetic construct
  REGION 1..15: note = epitope
LPDKGRVVIESEKKE

SEQ ID NO: 875 - protein, 15 aa, synthetic construct
  REGION 1..15: note = epitope
  VARIANT 7..8: note = any amino acid
  VARIANT 11..15: note = any amino acid
ATKAKDXXREXXXXX

SEQ ID NO: 876 - protein, 15 aa, synthetic construct
  REGION 1..15: note = epitope
  VARIANT 11..15: note = any amino acid
ATKAKDVTREXXXXX

SEQ ID NO: 877 - protein, 15 aa, synthetic construct
  REGION 1..15: note = epitope
ATKAKDVTREKGQQG

SEQ ID NO: 878 - protein, 15 aa, synthetic construct
  REGION 1..15: note = epitope
  VARIANT 1: note = any amino acid
  VARIANT 3: note = any amino acid
  VARIANT 7: note = any amino acid
  VARIANT 10..15: note = any amino acid
XGXKGQXKKXXXXXX

SEQ ID NO: 879 - protein, 15 aa, synthetic construct
  REGION 1..15: note = epitope
  VARIANT 1: note = any amino acid
  VARIANT 3: note = any amino acid
  VARIANT 10..11: note = any amino acid
XGXKGQTKKXXGEEQ

SEQ ID NO: 880 - protein, 15 aa, synthetic construct
  REGION 1..15: note = epitope
AGTKGQTKKGGGEEQ

SEQ ID NO: 881 - protein, 10 aa, synthetic construct
  REGION 1..10: note = epitope
  VARIANT 2..4: note = any amino acid
  VARIANT 10: note = any amino acid
RXXXETKEGX

SEQ ID NO: 882 - protein, 10 aa, synthetic construct
  REGION 1..10: note = epitope
REKAETKEGG

SEQ ID NO: 883 - protein, 10 aa, synthetic construct
  REGION 1..10: note = epitope
  VARIANT 1..4: note = any amino acid
XXXXNKQSSL

SEQ ID NO: 884 - protein, 10 aa, synthetic construct
  REGION 1..10: note = epitope
IGEENKQSSL

SEQ ID NO: 885 - protein, 15 aa, synthetic construct
  REGION 1..15: note = epitope
  VARIANT 1: note = any amino acid
  VARIANT 3: note = any amino acid
  VARIANT 6..13: note = any amino acid
  VARIANT 15: note = any amino acid
XKXVAXXXXXXXXEX

SEQ ID NO: 886 - protein, 15 aa, synthetic construct
  REGION 1..15: note = epitope
  VARIANT 1: note = any amino acid
  VARIANT 3: note = any amino acid
  VARIANT 7..9: note = any amino acid
  VARIANT 11: note = any amino acid
XKXVAEXXXQXASEL

SEQ ID NO: 887 - protein, 15 aa, synthetic construct
  REGION 1..15: note = epitope
VKGVAEYAGQKASEL

SEQ ID NO: 888 - protein, 10 aa, synthetic construct
  REGION 1..10: note = epitope
  VARIANT 1..2: note = any amino acid
  VARIANT 8..10: note = any amino acid
XXKVEAEXXX

SEQ ID NO: 889 - protein, 10 aa, synthetic construct
  REGION 1..10: note = epitope
RKKVEAEMDL

SEQ ID NO: 890 - protein, 10 aa, synthetic construct
  REGION 1..10: note = epitope
  VARIANT 1..4: note = any amino acid
XXXXIAEIAE

SEQ ID NO: 891 - protein, 10 aa, synthetic construct
  REGION 1..10: note = epitope
  VARIANT 1..2: note = any amino acid
XXETIAEIAE

SEQ ID NO: 892 - protein, 10 aa, synthetic construct
  REGION 1..10: note = epitope
IGETIAEIAE

SEQ ID NO: 893 - protein, 15 aa, synthetic construct
  REGION 1..15: note = epitope
  VARIANT 1: note = any amino acid
  VARIANT 3..4: note = any amino acid
  VARIANT 6: note = any amino acid
  VARIANT 8..11: note = any amino acid
  VARIANT 15: note = any amino acid
XYXXSXKXXXXEKVX

SEQ ID NO: 894 - protein, 15 aa, synthetic construct
  REGION 1..15: note = epitope
  VARIANT 1: note = any amino acid
  VARIANT 4: note = any amino acid
  VARIANT 6: note = any amino acid
  VARIANT 8..11: note = any amino acid
  VARIANT 15: note = any amino acid
XYIXSXKXXXXEKVX

SEQ ID NO: 895 - protein, 15 aa, synthetic construct
  REGION 1..15: note = epitope
LYIASVKANAVEKVE

SEQ ID NO: 896 - protein, 5 aa, synthetic construct
  REGION 1..5: note = epitope
  VARIANT 3: note = any amino acid
YEXGV

SEQ ID NO: 897 - protein, 5 aa, synthetic construct
  REGION 1..5: note = epitope
  VARIANT 3: note = any amino acid
YEXGV

SEQ ID NO: 898 - protein, 5 aa, synthetic construct
  REGION 1..5: note = epitope
YEAGV

SEQ ID NO: 899 - protein, 15 aa, synthetic construct
  REGION 1..15: note = epitope
AREAAARDAREAAAR

SEQ ID NO: 900 - skipped

SEQ ID NO: 901 - protein, 9 aa, synthetic construct
  REGION 1..9: note = epitope
  VARIANT 2..5: note = any amino acid
  VARIANT 9: note = any amino acid
VXXXXVTAX

SEQ ID NO: 902 - protein, 9 aa, synthetic construct
  REGION 1..9: note = epitope
VKRTTVTAT

SEQ ID NO: 903 - protein, 15 aa, synthetic construct
  REGION 1..15: note = epitope
AWGVEPYDLDDADRN

SEQ ID NO: 904 - protein, 15 aa, synthetic construct
  REGION 1..15: note = epitope
  VARIANT 1..2: note = any amino acid
  VARIANT 4..10: note = any amino acid
  VARIANT 14..15: note = any amino acid
XXWXXXXXXXEKEXX

SEQ ID NO: 905 - protein, 15 aa, synthetic construct
  REGION 1..15: note = epitope
  VARIANT 1..2: note = any amino acid
  VARIANT 4..7: note = any amino acid
  VARIANT 9..10: note = any amino acid
  VARIANT 15: note = any amino acid
XXWXXXXEXXEKESX

SEQ ID NO: 906 - protein, 15 aa, synthetic construct
  REGION 1..15: note = epitope
INWKYASEVYEKESS

SEQ ID NO: 907 - protein, 15 aa, synthetic construct
  REGION 1..15: note = epitope
  VARIANT 1..4: note = any amino acid
  VARIANT 12: note = any amino acid
XXXXEEEREHAXQLI

SEQ ID NO: 908 - protein, 15 aa, synthetic construct
  REGION 1..15: note = epitope
KESSEEEREHAEQLI

SEQ ID NO: 909 - protein, 15 aa, synthetic construct
  REGION 1..15: note = epitope
KESSEEEREHAEQLI

SEQ ID NO: 910 - protein, 15 aa, synthetic construct
  REGION 1..15: note = epitope
PDGILAYLDEENKPQ

SEQ ID NO: 911 - protein, 15 aa, synthetic construct
  REGION 1..15: note = epitope
CSLKHVPGFIERAEE

SEQ ID NO: 912 - protein, 15 aa, synthetic construct
  REGION 1..15: note = epitope
ALGLELDLTDKGLGV

SEQ ID NO: 913 - skipped

SEQ ID NO: 914 - protein, 9 aa, synthetic construct
  REGION 1..9: note = epitope
  VARIANT 1: note = any amino acid
  VARIANT 5..7: note = any amino acid
  VARIANT 9: note = any amino acid
XDIDXXXDX

SEQ ID NO: 915 - protein, 9 aa, synthetic construct
  REGION 1..9: note = epitope
WDIDYQLDL

SEQ ID NO: 916 - protein, 15 aa, synthetic construct
  REGION 1..15: note = epitope
KADAGASKTYPQQAG

SEQ ID NO: 917 - protein, 15 aa, synthetic construct
  REGION 1..15: note = epitope
CDVPHVNRTDYQLID

SEQ ID NO: 918 - protein, 15 aa, synthetic construct
  REGION 1..15: note = epitope
FAEGKDLVVSVMSAM

SEQ ID NO: 919 - skipped

SEQ ID NO: 920 - protein, 9 aa, synthetic construct
  REGION 1..9: note = epitope
  VARIANT 1..4: note = any amino acid
  VARIANT 6: note = any amino acid
XXXXPXFDK

SEQ ID NO: 921 - protein, 9 aa, synthetic construct
  REGION 1..9: note = epitope
KAEFPIFDK

SEQ ID NO: 922 - protein, 15 aa, synthetic construct
  REGION 1..15: note = epitope
WNFSKFLVDKDGNVV

SEQ ID NO: 923 - protein, 5 aa, synthetic construct
  REGION 1..5: note = epitope
KSRDD

SEQ ID NO: 924 - protein, 15 aa, synthetic construct
  REGION 1..15: note = epitope
PSQPAESKGEESGGG

SEQ ID NO: 925-926 - skipped (2 entries)

SEQ ID NO: 927 - protein, 9 aa, synthetic construct
  REGION 1..9: note = epitope
KLDDKQGIG

SEQ ID NO: 928 - protein, 15 aa, synthetic construct
  REGION 1..15: note = epitope
SIEQKEESRGNEDHV

SEQ ID NO: 929 - protein, 15 aa, synthetic construct
  REGION 1..15: note = epitope
FKTGAERKEAAESTL

SEQ ID NO: 930 - protein, 15 aa, synthetic construct
  REGION 1..15: note = epitope
ELDTLGEESYKDSTL

SEQ ID NO: 931 - skipped

SEQ ID NO: 932 - protein, 15 aa, synthetic construct
  REGION 1..15: note = epitope
  VARIANT 1..2: note = any amino acid
  VARIANT 4..6: note = any amino acid
  VARIANT 8..9: note = any amino acid
  VARIANT 11: note = any amino acid
  VARIANT 13..15: note = any amino acid
XXGXXXSXXEXLXXX

SEQ ID NO: 933 - protein, 15 aa, synthetic construct
  REGION 1..15: note = epitope
GTGLIVSFGEMLIDF

SEQ ID NO: 934 - protein, 15 aa, synthetic construct
  REGION 1..15: note = epitope
DEFGHMLAGILKENG

SEQ ID NO: 935 - protein, 15 aa, synthetic construct
  REGION 1..15: note = epitope
SADMLLKPEELNLEL

SEQ ID NO: 936 - protein, 15 aa, synthetic construct
  REGION 1..15: note = epitope
DLIKVSDAELEFLTG

SEQ ID NO: 937 - protein, 15 aa, synthetic construct
  REGION 1..15: note = epitope
EFLTGSDKIDDESAL

SEQ ID NO: 938 - protein, 15 aa, synthetic construct
  REGION 1..15: note = epitope
VDDQSILEDEPRLRE

SEQ ID NO: 939 - protein, 15 aa, synthetic construct
  REGION 1..15: note = epitope
KGGAYTGEVSAEMLV

SEQ ID NO: 940 - protein, 15 aa, synthetic construct
  REGION 1..15: note = epitope
GGGKPSGELLELIER

SEQ ID NO: 941-942 - skipped (2 entries)

SEQ ID NO: 943 - protein, 7 aa, synthetic construct
  REGION 1..7: note = epitope
KAAAATQ

SEQ ID NO: 944 - protein, 9 aa, synthetic construct
  REGION 1..9: note = epitope
  VARIANT 3..5: note = any amino acid
  VARIANT 8..9: note = any amino acid
TKXXXSDXX

SEQ ID NO: 945 - protein, 9 aa, synthetic construct
  REGION 1..9: note = epitope
  VARIANT 4..5: note = any amino acid
  VARIANT 8..9: note = any amino acid
TKDXXSDXX

SEQ ID NO: 946 - protein, 9 aa, synthetic construct
  REGION 1..9: note = epitope
TKDYASDAA

SEQ ID NO: 947 - protein, 8 aa, synthetic construct
  REGION 1..8: note = epitope
  VARIANT 4..6: note = any amino acid
VKDXXXDA

SEQ ID NO: 948 - protein, 8 aa, synthetic construct
  REGION 1..8: note = epitope
  VARIANT 5..6: note = any amino acid
VKDYXXDA

SEQ ID NO: 949 - protein, 8 aa, synthetic construct
  REGION 1..8: note = epitope
VKDYASDA

SEQ ID NO: 950 - skipped

SEQ ID NO: 951 - protein, 9 aa, synthetic construct
  REGION 1..9: note = epitope
  VARIANT 3..7: note = any amino acid
KDXXXXXAQ

SEQ ID NO: 952 - protein, 9 aa, synthetic construct
  REGION 1..9: note = epitope
KDYASDTAQ

SEQ ID NO: 953 - protein, 15 aa, synthetic construct
  REGION 1..15: note = epitope
  VARIANT 1..7: note = any amino acid
  VARIANT 9: note = any amino acid
  VARIANT 11: note = any amino acid
  VARIANT 13: note = any amino acid
XXXXXXXEXYXLXHP

SEQ ID NO: 954 - protein, 15 aa, synthetic construct
  REGION 1..15: note = epitope
  VARIANT 1..6: note = any amino acid
XXXXXXIEAYLLAHP

SEQ ID NO: 955 - protein, 15 aa, synthetic construct
  REGION 1..15: note = epitope
DALFKAIEAYLLAHP

SEQ ID NO: 956 - protein, 9 aa, synthetic construct
  REGION 1..9: note = epitope
QKESEERKQ

SEQ ID NO: 957 - protein, 8 aa, synthetic construct
  REGION 1..8: note = epitope
SYRLQSGD

SEQ ID NO: 958 - protein, 9 aa, synthetic construct
  REGION 1..9: note = epitope
DALRVPSGT

SEQ ID NO: 959 - protein, 9 aa, synthetic construct
  REGION 1..9: note = epitope
RKTISSEDK

SEQ ID NO: 960 - protein, 15 aa, synthetic construct
  REGION 1..15: note = epitope
QHPEREPQQPGEKEE

SEQ ID NO: 961 - protein, 15 aa, synthetic construct
  REGION 1..15: note = epitope
GHQSQKGKHQQEEEN

SEQ ID NO: 962 - protein, 15 aa, synthetic construct
  REGION 1..15: note = epitope
LLNALPEEVIQHTFN

SEQ ID NO: 963 - protein, 15 aa, synthetic construct
  REGION 1..15: note = epitope
MRGRINYIRRNEGKD

SEQ ID NO: 964 - protein, 20 aa, synthetic construct
  REGION 1..20: note = epitope candidate
SSPRGKVEAEKARPMEETKV

SEQ ID NO: 965 - protein, 10 aa, synthetic construct
  REGION 1..10: note = epitope candidate
SSPRGKVEAE

SEQ ID NO: 966 - protein, 10 aa, synthetic construct
  REGION 1..10: note = epitope candidate
SPRGKVEAEK

SEQ ID NO: 967 - protein, 10 aa, synthetic construct
  REGION 1..10: note = epitope candidate
PRGKVEAEKA

SEQ ID NO: 968 - protein, 10 aa, synthetic construct
  REGION 1..10: note = epitope candidate
RGKVEAEKAR

SEQ ID NO: 969 - protein, 10 aa, synthetic construct
  REGION 1..10: note = epitope candidate
GKVEAEKARP

SEQ ID NO: 970 - protein, 10 aa, synthetic construct
  REGION 1..10: note = epitope candidate
KVEAEKARPM

SEQ ID NO: 971 - protein, 10 aa, synthetic construct
  REGION 1..10: note = epitope candidate
VEAEKARPME

SEQ ID NO: 972 - protein, 10 aa, synthetic construct
  REGION 1..10: note = epitope candidate
EAEKARPMEE

SEQ ID NO: 973 - protein, 10 aa, synthetic construct
  REGION 1..10: note = epitope candidate
AEKARPMEET

SEQ ID NO: 974 - protein, 10 aa, synthetic construct
  REGION 1..10: note = epitope candidate
EKARPMEETK

SEQ ID NO: 975 - protein, 10 aa, synthetic construct
  REGION 1..10: note = epitope candidate
KARPMEETKV

SEQ ID NO: 976 - protein, 8 aa, synthetic construct
  REGION 1..8: note = modified epitope
ARGKVEAE

SEQ ID NO: 977 - protein, 8 aa, synthetic construct
  REGION 1..8: note = modified epitope
PAGKVEAE

SEQ ID NO: 978 - protein, 8 aa, synthetic construct
  REGION 1..8: note = modified epitope
PRAKVEAE

SEQ ID NO: 979 - protein, 8 aa, synthetic construct
  REGION 1..8: note = modified epitope
PRGAVEAE

SEQ ID NO: 980 - protein, 8 aa, synthetic construct
  REGION 1..8: note = modified epitope
PRGKAEAE

SEQ ID NO: 981 - protein, 8 aa, synthetic construct
  REGION 1..8: note = modified epitope
PRGKVAAE

SEQ ID NO: 982 - protein, 8 aa, synthetic construct
  REGION 1..8: note = modified epitope
PRGKVEAE

SEQ ID NO: 983 - protein, 8 aa, synthetic construct
  REGION 1..8: note = modified epitope
PRGKVEAA

SEQ ID NO: 984 - protein, 15 aa, synthetic construct
  REGION 1..15: note = epitope
MLSKQMRRRLSADER

SEQ ID NO: 985 - protein, 14 aa, synthetic construct
  REGION 1..14: note = epitope
ASDHVPRRSKTSLR